(19) United States
(12) Patent Application Publication
(10) Pub. No.: US 2021/0317140 A1
(43) Pub. Date: Oct. 14, 2021

(54) Heterocyclic Compounds and Methods of Use

(71) Applicant: MEDIVATION TECHNOLOGIES, INC, San Francisco, CA (US)

(72) Inventors: Roopa RAI, San Carlos, CA (US); Sarvajit CHAKRAVARTY, Mountain View, CA (US); Michael John GREEN, Half Moon Bay, CA (US); Son Minh PHAM, San Francisco, CA (US); Brahman PUJALA, Noida (IN); Anil Kumar AGARWAL, Noida (IN); Anjan Kumar NAYAK, Noida (IN); Sweta KHARE, Noida (IN); Rambabu GUGULOTH, Noida (IN); Nitin Atmaram RANDIVE, Noida (IN)

(73) Assignee: Medivation Technologies, Inc., San Francisco, CA (US)

(21) Appl. No.: 15/030,042

(22) PCT Filed: Oct. 17, 2014
(86) PCT No.: PCT/US2014/061136

§ 371 Date: Oct. 7, 2016

(30) Foreign Application Priority Data
Oct. 18, 2013 (IN) 4714/CHE/2013

(51) Int. Cl. C07D 519/00; C07D 487/04

(52) U.S. Cl. CPC C07D 519/00; C07D 487/04

(57) ABSTRACT

The present application discloses compounds that are inhibitors of Btk, compounds that are inhibitors of PI3Kδ, and compounds that are dual inhibitors of both Btk and PI3Kδ. Also described are methods for synthesizing such inhibitors and methods for using such inhibitors for the treatment of diseases wherein inhibition of Btk and PI3Kδ provides a therapeutic benefit to a patient having the disease.

### BACKGROUND

Despite the many advances in treating autoimmune diseases and hematologic diseases, there remains a need for effective therapies to treat such diseases. Bruton's tyrosine kinase (Btk) is a Tec family kinase known for its role in B-cell antigen receptor (BCR) signaling. Activation of this BCR signaling pathway leads to various B-cell malignancies and autoimmune diseases, including arthritis, lupus, multiple sclerosis, B-cell lymphomas, and leukemia such as chronic lymphocytic leukemia (CLL). The development of small molecule Btk inhibitors may provide therapeutic benefits for the treatment of leukemia, lymphoma and such autoimmune diseases [Honigberg, et al. Proc. Natl. Acad. Sci. (2010), 107:29, pp 13075-13080; Barrientos, et al. Leukemia & Lymphoma (August 2013), 54:8, pp 1817-1820; Li, et al. J. Med. Chem. (2014), 57:12, pp 5112-5128]. Inhibition of the BCR signaling pathway has several intervention points: at SYK, Btk, and PI3K6. Phosphoinositide 3-kinases (PI3Ks) constitute a family of enzymes widely involved in cell signaling and controlling a broad number of cellular processes including cell proliferation, survival, motility and metabolism. They comprise 4 isoforms: α, β, γ, and δ, each of which has been studied extensively with potential small molecule inhibitors of pan-PI3K, or more preferentially for selective isoforms, having been developed [Ciraolo, et al. Curr. Med. Chem. (2011), 18, pp 2674-2685]. The PI3Kδ isoform has proved of particular interest as a central signaling enzyme that mediates the effects of multiple receptors on B-cells. PI3Kδ signaling is important for B-cell survival, migration and activation, and PI3Kδ kinase activation is believed to be involved in a range of cellular responses including cell growth, differentiation and apoptosis. Targeting of PI3Kδ is thus an attractive means of controlling aberrant B-cell activation [Puri, et al. Frontiers in Immunology (August 2012), 3:256, pp 1-16]. Potential therapies targeting aberrant BCR signaling, including the individual inhibition of PI3K, SYK, or BTK, have been summarized recently [Choi, et al. Cancer J. (2012), 18, pp 404-410; Rickert, R. C. Nat. Rev. Immunol. (2013), 13:8, pp 578-591; Cushing, et al. J. Med. Chem. (2012), 55, pp 8559-8581; Xing, et al. Future Med. Chem. (2014), 6:6, pp 675-695; Shinohara, et al. Bone (2014), 60: pp 8-15]. The capability to inhibit at more than one of the intervention points with a single molecule could provide a synergistic response resulting in increased therapeutic efficacy and reduced resistance often observed with inhibition of a single kinase. As such, dual inhibitors of BTK and PI3Kδ should overcome resistance due to SYK, BTK and PI3Kδ monotherapy, and that such dual inhibitors could inhibit SYK-independent proliferation of CLL cells.

### BRIEF SUMMARY

Described herein are compounds that are dual inhibitors of both Btk and PIKδ. Also described are methods for synthesizing such inhibitors and methods for using such inhibitors for the treatment of diseases wherein inhibition of Btk and PI3Kδ provides a therapeutic benefit to a patient having the disease. In one aspect, the compounds are reversible inhibitors of both BTK and PIKδ. In another aspect, the compounds are irreversible inhibitors of both BTK and PIKδ.

### DETAILED DESCRIPTION

### Definitions

For use herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”.

Unless clearly indicated otherwise, “an individual” as used herein intends a mammal, including but not limited to a human, bovine, primate, equine, canine, feline, porcine, and ovine animals. Thus, the compositions and methods provided herein use in both human medicine and in the veterinary context, including use in agricultural animals and domestic pets. The individual may be a human who has been diagnosed with or is suspected of having cancer. The individual may be a human who exhibits one or more symptoms associated with cancer. The individual may be a human who has a mutated or abnormal gene associated with cancer. The individual may be a human who is genetically or otherwise predisposed to developing cancer.

As used herein, “treatment” or “treating” is an approach for obtaining beneficial or desired results including clinical results. For purposes of the compositions and methods provided herein, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread (e.g., metastasis) of the disease, delaying or slowing the progression of the disease, ameliorating the disease state, providing a remission (whether partial or total) of the disease, decreasing the dose of one or more other medications required to treat the disease, enhancing the effect of another medication used to treat the disease, increasing the quality of life of an individual having the disease, and/or prolonging survival. A method of treating cancer encompasses a reduction of the pathological consequence of cancer. The methods described herein contemplate any one or more of these aspects of treatment.

As used herein, “delaying” the development of a disease or condition such as cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, such as routine physical exams, mammography, imaging, or biopsy. Development may also refer to disease progression that may be initially undetectable and includes occurrence, recurrence, and onset.

As used herein, an “at risk” individual is an individual who is at risk of developing cancer. An individual “at risk” may or may not have detectable disease, and may or may not have displayed detectable disease prior to the treatment methods described herein. “At risk” denotes that an individual has one or more so-called risk factors, which are measurable parameters that correlate with development of cancer, which are described herein. An individual having one or more of these risk factors has a higher probability of developing cancer than an individual without these risk factor(s).

As used herein, by “combination therapy” is meant a therapy that includes two or more different compounds. Thus, in one aspect, a combination therapy comprising a compound detailed herein and another compound is provided. In some embodiments, the combination therapy optionally includes one or more pharmaceutically acceptable carriers or excipients, non-pharmaceutically active compounds, and/or inert substances. In various embodiments, treatment with a combination therapy may result in an additive or even synergistic (e.g., greater than additive) result compared to administration of a single compound provided herein alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency, and/or duration of one or more side-effects associated with the compound.

As used herein, the term “effective amount” intends such amount of a compound provided herein which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds. In various embodiments, an effective amount of the composition or therapy may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

As is understood in the art, an “effective amount” may be in one or more doses, i.e., a single dose or multiple doses may be required to achieve the desired treatment endpoint. An effective amount may be considered in the context of administering one or more therapeutic agents, and a compound, or pharmaceutically acceptable salt thereof, may be considered to be given in an effective amount if, in conjunction with one or more other agents, a desirable or beneficial result may be or is achieved.

A “therapeutically effective amount” refers to an amount of a compound or salt thereof sufficient to produce a desired therapeutic outcome (e.g., reducing the severity or duration of, stabilizing the severity of, or eliminating one or more symptoms of cancer). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients.

A “prophylactically effective amount” refers to an amount of a compound, or pharmaceutically acceptable salt thereof, sufficient to prevent or reduce the severity of one or more future symptoms of cancer when administered to an individual who is susceptible and/or who may develop cancer. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotypes presenting during future development of the disease).

It is understood that an effective amount of a compound or pharmaceutically acceptable salt thereof, including a prophylactically effective amount, may be given to an individual in the adjuvant setting, which refers to a clinical setting in which an individual has had a history of cancer, and generally (but not necessarily) has been responsive to therapy, which includes, but is not limited to, surgery (e.g., surgical resection), radiotherapy, and chemotherapy. However, because of their history of the cancer, these individuals are considered at risk of developing cancer. Treatment or administration in the “adjuvant setting” refers to a subsequent mode of treatment.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound provided herein in its free acid or base form with a suitable organic or inorganic base or acid, respectively, and isolating the salt thus formed during subsequent purification. A pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “excipient” as used herein means an inert or inactive substance that may be used in the production of a drug or pharmaceutical, such as a tablet containing a compound provided herein as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Alkyl” refers to and includes saturated linear or branched univalent hydrocarbon structures and combinations thereof. Particular alkyl groups are those having 1 to 20 carbon atoms (a “C₁-C₂₀ alkyl”). More particular alkyl groups are those having 1 to 8 carbon atoms (a “C₁-C₈ alkyl”). When an alkyl residue having a specific number of carbons is named, all geometric isomers having that number of carbons are intended to be encompassed and described; thus, for example, “butyl” is meant to include n-butyl, sec-butyl, iso-butyl, and tert-butyl; “propyl” includes n-propyl and iso-propyl. This term is exemplified by groups such as methyl, t-butyl, n-heptyl, octyl, and the like.

“Cycloalkyl” refers to and includes cyclic univalent hydrocarbon structures. Cycloalkyl can consist of one ring, such as cyclohexyl, or multiple rings, such as adamantyl. A cycloalkyl comprising more than one ring may be fused, spiro or bridged, or combinations thereof. A preferred cycloalkyl is a saturated cyclic hydrocarbon having from 3 to 13 annular carbon atoms. A more preferred cycloalkyl is a saturated cyclic hydrocarbon having from 3 to 8 annular carbon atoms (a “C₃-C₈ cycloalkyl”). Examples of cycloalkyl groups include adamantyl, decahydronaphthalenyl, cyclopropyl, cyclobutyl, cyclopentyl, cyclohexyl, and the like.

“Alkylene” refers to the same residues as alkyl, but having bivalency. Examples of alkylene include methylene (—CH₂—), ethylene (—CH₂CH₂—), propylene (—CH₂CH₂CH₂—), butylene (—CH₂CH₂CH₂CH₂—) and the like.

“Alkenyl” refers to an unsaturated hydrocarbon group having at least one site of olefinic unsaturation (i.e., having at least one moiety of the formula C═C) and preferably having from 2 to 10 carbon atoms and more preferably 2 to 8 carbon atoms. Examples of alkenyl include but are not limited to —CH₂—CH═CH—CH₃ and —CH═CH—CH═CH₂.

“Cycloalkenyl” refers to an unsaturated hydrocarbon group within a cycloalkyl having at least one site of olefinic unsaturation (i.e., having at least one moiety of the formula C═C). Cycloalkenyl can consist of one ring, such as cyclohexyl, or multiple rings, such as norbornenyl. A more preferred cycloalkenyl is an unsaturated cyclic hydrocarbon having from 5 to 8 annular carbon atoms (a “C₅-C₈ cycloalkenyl”). Examples of cycloalkenyl groups include cyclopentenyl, cyclohexenyl, and the like.

“Alkynyl” refers to an unsaturated hydrocarbon group having at least one site of acetylenic unsaturation (i.e., having at least one moiety of the formula CC) and preferably having from 2 to 10 carbon atoms and more preferably 2 to 8 carbon atoms and the like.

“Substituted alkyl” refers to an alkyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, substituted or unsubstituted cycloalkyl, substituted or unsubstituted cycloalkenyl, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted cycloalkyl” refers to a cycloalkyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, substituted or unsubstituted cycloalkenyl, aryl, substituted aryl, heteroaryl, substituted heteroaryl, aryloxy, substituted aryloxy, cyano, halo, hydroxyl, nitro, carboxyl, thiol, thioalkyl, substituted or unsubstituted cycloalkyl, substituted or unsubstituted cycloalkenyl, substituted or unsubstituted alkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted alkenyl” refers to alkenyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted cycloalkenyl” refers to a cycloalkenyl group having from 1 to 5 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted alkynyl” refers to alkynyl groups having from 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted heterocyclyl, substituted or unsubstituted aralkyl, aminosulfonyl, sulfonylamino, sulfonyl, oxo, alkoxyalkylenecarbonyl and the like.

“Acyl” refers to the groups H—C(O)—, alkyl-C(O)—, substituted alkyl-C(O)—, alkenyl-C(O)—, substituted alkenyl-C(O)—, cycloalkyl-C(O)—, substituted cycloalkyl-C(O)—, cycloalkenyl-C(O)—, substituted cycloalkenyl-C(O)—, alkynyl-C(O)—, substituted alkynyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups H—C(O)O—, alkyl-C(O)O—, substituted alkyl-C(O)O—, alkenyl-C(O)O—, substituted alkenyl-C(O)O—, cycloalkenyl-C(O)O—, substituted cycloalkenyl-C(O)O—, alkynyl-C(O)O—, substituted alkynyl-C(O)O—, cycloalkyl-C(O)O—, substituted cycloalkyl-C(O)O—, aryl-C(O)O—, substituted aryl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, cycloalkenyl, substituted cycloalkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Heterocycle”, “heterocyclic”, or “heterocyclyl” refers to a saturated or an unsaturated non-aromatic group having a single ring or multiple condensed rings, and having from 1 to 10 annular carbon atoms and from 1 to 4 annular heteroatoms, such as nitrogen, sulfur or oxygen, and the like. A heterocycle comprising more than one ring may be fused, spiro or bridged, or any combination thereof. In fused ring systems, one or more of the rings can be aryl or heteroaryl. A heterocycle having more than one ring where at least one ring is aromatic may be connected to the parent structure at either a non-aromatic ring position or at an aromatic ring position. In one variation, a heterocycle having more than one ring where at least one ring is aromatic is connected to the parent structure at a non-aromatic ring position.

“Substituted heterocyclic” or “substituted heterocyclyl” refers to a heterocycle group which is substituted with from 1 to 3 substituents including, but not limited to, substituents such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted aralkyl, aminosulfonyl, sulfonylamino, sulfonyl, oxo, alkoxyalkylenecarbonyl and the like. In one variation, a substituted heterocycle is a heterocycle substituted with an additional ring, wherein the additional ring may be aromatic or non-aromatic.

“Aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” or “HetAr” refers to an unsaturated aromatic carbocyclic group having from 1 to 10 annular carbon atoms and at least one annular heteroatom, including but not limited to heteroatoms such as nitrogen, oxygen and sulfur. A heteroaryl group may have a single ring (e.g., pyridyl, furyl) or multiple condensed rings (e.g., indolizinyl, benzothienyl) which condensed rings may or may not be aromatic. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

“Substituted aryl” refers to an aryl group having 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Substituted heteroaryl” refers to a heteroaryl group having 1 to 5 substituents including, but not limited to, groups such as alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted alkyl, substituted or unsubstituted cycloalkyl, substituted or unsubstituted alkenyl, substituted or unsubstituted cycloalkenyl, substituted or unsubstituted alkynyl, substituted or unsubstituted heterocyclyl, substituted or unsubstituted aralkyl, aminosulfonyl, sulfonylamino, sulfonyl, oxo, alkoxyalkylenecarbonyl and the like.

“Aralkyl” refers to a residue in which an aryl moiety is attached to an alkyl residue and wherein the aralkyl group may be attached to the parent structure at either the aryl or the alkyl residue. Preferably, an aralkyl is connected to the parent structure via the alkyl moiety. In one variation, an aralkyl is a fused ring system where at least one cycloalkyl moiety is fused with at least one aryl moiety. A “substituted aralkyl” refers to a residue in which an aryl moiety is attached to a substituted alkyl residue and wherein the aralkyl group may be attached to the parent structure at either the aryl or the alkyl residue. When an aralkyl is connected to the parent structure via the alkyl moiety, it may also be referred to as an “alkaryl”. More particular alkaryl groups are those having 1 to 3 carbon atoms in the alkyl moiety (a “C₁-C₃ alkaryl”).

“Alkoxy” refers to the group alkyl-O—, which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like. Similarly, alkenyloxy refers to the group “alkenyl-O—” and alkynyloxy refers to the group “alkynyl-O—”. “Substituted alkoxy” refers to the group substituted alkyl-O.

“Unsubstituted amino” refers to the group —NH₂.

“Substituted amino” refers to the group —NRₐR_{b}, where either (a) each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic, provided that both Rₐ and R_{b} groups are not H; or (b) Rₐ and R_{b} are joined together with the nitrogen atom to form a heterocyclic or substituted heterocyclic ring.

“Aminoacyl” refers to the group —C(O)NRₐR_{b} where Rₐ and R_{b} are independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic or Rₐ and R_{b} groups can be joined together with the nitrogen atom to form a heterocyclic or substituted heterocyclic ring.

“Acylamino” refers to the group —NRₐC(O)R_{b} where each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic or substituted heterocyclic. Preferably, Rₐ is H or alkyl.

“Sulfonylamino” refers to the groups —NRSO₂-alkyl, —NRSO₂-substituted alkyl, —NRSO₂-alkenyl, —NRSO₂-substituted alkenyl, —NRSO₂-cycloalkenyl, —NRSO₂-substituted cycloalkenyl, —NRSO₂-alkynyl, —NRSO₂-substituted alkynyl, —NRSO₂-cycloalkyl, —NRSO₂— substituted cycloalkyl, —NRSO₂-aryl, —NRSO₂-substituted aryl, —NRSO₂-heteroaryl, —NRSO₂— substituted heteroaryl, —NRSO₂-heterocyclic, and —NRSO₂-substituted heterocyclic, where R is H or alkyl.

“Aminosulfonyl” refers to the groups —SO₂NH₂, —SO₂NR-alkyl, —SO₂NR-substituted alkyl, —SO₂NR-cycloalkyl, —SO₂NR-substituted cycloalkyl, —SO₂NR-alkenyl, —SO₂NR-substituted alkenyl, —SO₂NR-cycloalkenyl, —SO₂NR-substituted cycloalkenyl, —SO₂NR-alkynyl, —SO₂NR-substituted alkynyl, —SO₂NR-aryl, —SO₂NR-substituted aryl, —SO₂NR-heteroaryl, —SO₂NR-substituted heteroaryl, —SO₂NR-heterocyclic, and —SO₂NR-substituted heterocyclic, where R is H or alkyl, or —SO₂NR₂, where the two R groups are taken together and with the nitrogen atom to which they are attached to form a heterocyclic or substituted heterocyclic ring.

“Sulfonyl” refers to the groups —SO₂-alkyl, —SO₂-substituted alkyl, —SO₂-cycloalkyl, —SO₂-substituted cycloalkyl, —SO₂-alkenyl, —SO₂-substituted alkenyl, —SO₂-cycloalkenyl, —SO₂-substituted cycloalkenyl, —SO₂-alkynyl, —SO₂-substituted alkynyl, —SO₂-aryl, —SO₂— substituted aryl, —SO₂-heteroaryl, —SO₂-substituted heteroaryl, —SO₂-heterocyclic, and —SO₂-substituted heterocyclic.

“Alkoxycarbonylamino” refers to the group —NRₐC(O)OR_{b} where each Rₐ and R_{b} group is independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic and substituted heterocyclyl.

“Alkoxyalkylenecarbonyl” refers to the group —C(═O)—(CH₂)ₙ—OR where R is a substituted or unsubstituted alkyl and n is an integer from 1 to 100, more preferably n is an integer from 1 to 10 or 1 to 5.

“Halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl, etc., refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each H is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Cyano” refers to the group —CN.

“Oxo” refers to the moiety ═O.

“Nitro” refers to the group —NO₂.

“Thioalkyl” refers to the groups —S-alkyl.

“Thio” refers to the group —S— that may be a —SH (a thiol or an unsubstituted thio group) or selected from the groups —S-alkyl, —S-alkenyl, —S-alkynyl, —S-cycloalkyl, —S— heterocyclyl, —S-aryl and —S-heteroaryl.

“Aminosulfonylalkylene” refers to the group —R¹SO₂NRₐR_{b} where Rₐ and R_{b} are independently selected from the group consisting of H, alkyl, substituted alkyl, cycloalkyl, substituted cycloalkyl, alkenyl, substituted alkenyl, cycloalkenyl, substituted cycloalkenyl, alkynyl, substituted alkynyl, aryl, substituted aryl, heteroaryl, substituted heteroaryl, heterocyclic, substituted heterocyclic, or the Rₐ and R_{b} groups can be joined together with the nitrogen atom to form a heterocyclic or substituted heterocyclic ring and R¹ is an alkylene group.

“Alkoxycarbonyl” refers to as used herein refers to the groups —C(O)O-alkyl, —C(O)O-substituted alkyl, —C(O)O-cycloalkyl, —C(O)O-substituted cycloalkyl, —C(O)O-aryl, —C(O)O-substituted aryl, —C(O)O-alkenyl, —C(O)O-substituted alkenyl, —C(O)O-cycloalkenyl, —C(O)O-substituted cycloalkenyl, —C(O)O-alkynyl, —C(O)O-substituted alkynyl, —C(O)O— heteroaryl, —C(O)O-substituted heteroaryl, —C(O)O-heterocyclic or —C(O)O-substituted heterocyclic.

“Geminal” refers to the relationship between two moieties that are attached to the same atom. For example, in the residue —CH₂—CHR¹R², R¹ and R² are geminal and R¹ may be referred to as a geminal R group to R².

“Vicinal” refers to the relationship between two moieties that are attached to adjacent atoms. For example, in the residue —CH₂—CHR¹R², R¹ and R² are vicinal and R¹ may be referred to as a vicinal R group to R².

A composition of “substantially pure” compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

### Compounds of the Invention

Compounds according to the invention are detailed herein, including in the Brief Summary of the Invention and elsewhere. The invention includes the use of all of the compounds described herein, including any and all stereoisomers, including geometric isomers (cis/trans or E/Z isomers), tautomers, salts, N-oxides, and solvates of the compounds described herein, as well as methods of making such compounds.

In one aspect, provided are compounds of formula (I):

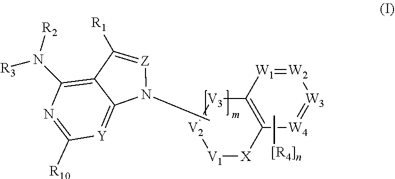

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ and —NHR₅;

V₁, V₂, and V₃, where present, are each independently:

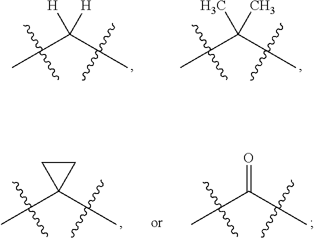

and one of V₁, V₂, or V₃ is

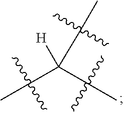 [SMILES: *C(*)*]

W₁, W₂, W₃ and W₄ are each independently CH or N, or CR₄ when R₄ is present;

X is CH₂ or NR₅;

Y is CH or N;

Z is N or CR₉;

m is 0 or 1;

n is 0 or an integer from 1 to 4;

R₅ is selected from the group consisting of H, —[C₁-C₆ alkyl]-CN,

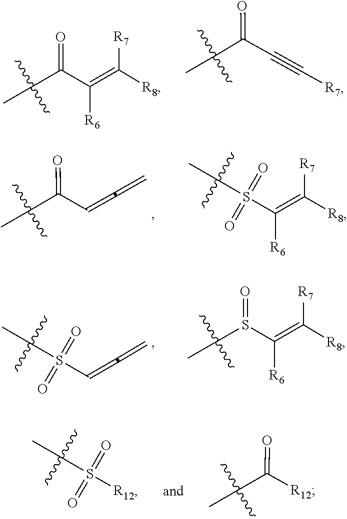

R₆ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂, and —C(═O)NH₂;

R₇ and R₈ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₉ is H, halo, CN, CF₃, —C(═O)NH₂, or substituted or unsubstituted C₁-C₆ alkyl;

R₁₀ is H, halo, —NHR₂, and substituted or unsubstituted C₁-C₆ alkyl; and

R₁₂ is substituted or unsubstituted C₁-C₆ alkyl or substituted or unsubstituted C₃-C₆ cycloalkyl.

In another aspect, provided are compounds of formula (II):

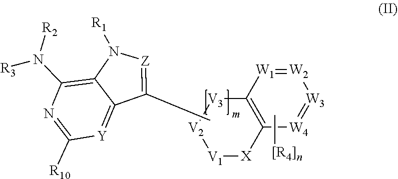

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl;

each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, and acyl;

V₁, V₂, and V₃, where present, are each independently selected from the group consisting of:

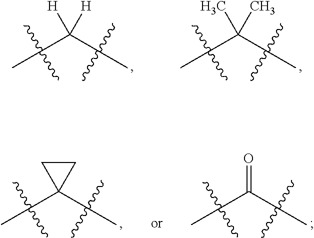

and one of V₁, V₂, or V₃ is

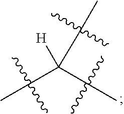 [SMILES: *C(*)*]

W₁, W₂, W₃ and W₄ are each independently CH or N, or CR₄ when R₄ is present;

X is CH₂ or NR₅;

Y is CH or N;

Z is N or CR₉;

m is 0 or 1;

n is 0 or an integer from 1 to 4;

R₅ is H, —[C₁-C₆ alkyl]-CN.

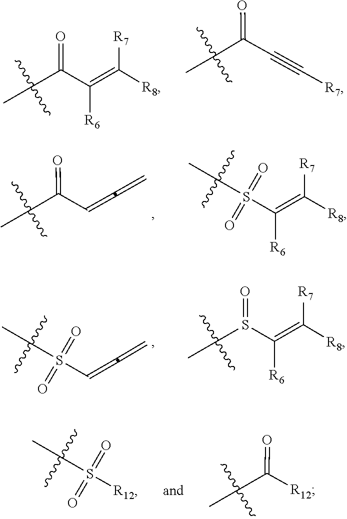

R₆ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂, and —C(═O)NH₂;

R₇ and R₈ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₉ is H, halo, CN, CF₃, —C(═O)NH₂, or substituted or unsubstituted C₁-C₆ alkyl;

R₁₀ is H, halo, —NHR₂ or substituted or unsubstituted C₁-C₆ alkyl; and

R₁₂ is substituted or unsubstituted C₁-C₆ alkyl or substituted or unsubstituted C₃-C₆ cycloalkyl.

In another aspect, provided are compounds of formula (III):

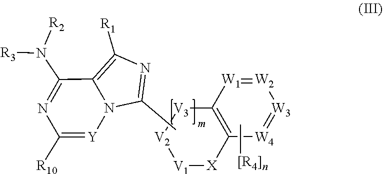

or a salt thereof;
wherein:

R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;

R₂ and R₃ are each independently H or substituted or unsubstituted C₁-C₆ alkyl; each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, and acyl;

V₁, V₂, and V₃, where present, are each independently selected from the group consisting of:

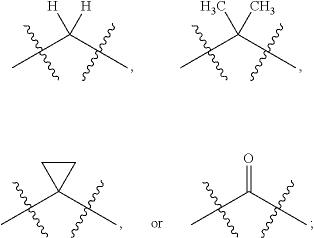

and one of V₁, V₂, or V₃ is

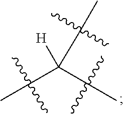 [SMILES: *C(*)*]

W₁, W₂, W₃ and W₄ are each CH or N, or CR₄ when R₄ is present;

X is CH₂ or NR₅;

Y is CH or N;

m is 0 or 1;

n is 0 or an integer from 1 to 4;

R₅ is H, —[C₁-C₆ alkyl]-CN,

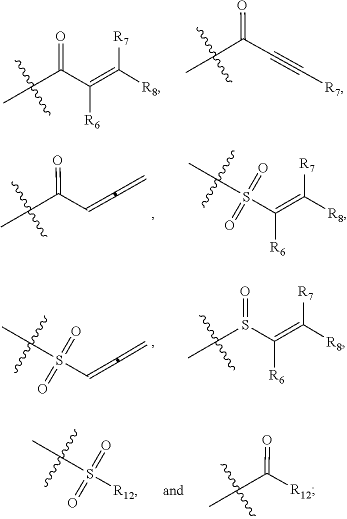

R₆ is H, or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂, and —C(═O)NH₂;

R₇ and R₈ are each independently H, or selected from the group consisting of substituted or unsubstituted C₁-C₆ alkyl, substituted or unsubstituted cycloalkyl, substituted or unsubstituted heterocyclyl, and substituted or unsubstituted amino;

R₁₀ is H, halo, —NHR₂ or substituted or unsubstituted C₁-C₆ alkyl; and R₁₂ is substituted or unsubstituted C₁-C₆ alkyl or substituted or unsubstituted C₃-C₆ cycloalkyl.

In all embodiments of formulae (I), (II) and (III), one of V₁, V₂, or V₃ where present, is bonded to the bicyclic heterocycle, which are the fused bicyclic rings containing the variable Y or the variables Y and Z. In some of these embodiments, each of the remaining V₁, V₂, and V₃ where present, is —CH₂—. In some embodiments, one or more of the remaining V₁, V₂, and V₃ where present, is —C(CH₃)₂—. In some embodiments, one of the remaining V₁, V₂, and V₃ where present, is —C(CH₂—CH₂)—. In some embodiments, one or more of the remaining V₁, V₂, and V₃ where present, is —C(═O)—.

In some embodiments of formulae (I), (II) and (III), V₁ is —C(═O)—, and X is NR₅, wherein R₅ is —[C₁-C₆ alkyl]-CN.

In some embodiments of formulae (I), (II) and (III), each W₁, W₂, W₃ and W₄ is CH. In some embodiments, at least one of W₁, W₂, W₃ and W₄ is CR₄, where R₄ is selected from the group consisting of hydroxyl, nitro, cyano, halo, C₁-C₈ perhaloalkyl, C₁-C₈ alkyl, C₃-C₈ cycloalkyl, C₂-C₈ alkenyl, C₅-C₈ cycloalkenyl, C₂-C₈ alkynyl, aryl, heteroaryl, C₁-C₈ perhaloalkoxy, C₁-C₈ alkoxy, aryloxy, carboxyl, thiol, heterocyclyl, aralkyl, thioalkyl, amino, acylamino, aminoacyl, aminocarbonylamino, alkoxycarbonylamino, aminosulfonyl, sulfonylamino, sulfonyl, alkoxyalkylenecarbonyl, aminosulfonylalkylene and acyl. In some embodiments, one of W₁, W₂, W₃ and W₄ is N. In some embodiments two non-adjacent W₁, W₂, W₃, and W₄ are N. In some embodiments, no more than two W₁, W₂, W₃ and W₄ are N.

In some embodiments of formulae (I), (II) and (III), R₁ is substituted or unsubstituted C₁-C₆ alkyl. In some embodiments, R₁ is substituted or unsubstituted C₂-C₆ alkenyl. In some embodiments, R₁ is substituted or unsubstituted aryl, substituted or unsubstituted heteroaryl, substituted or unsubstituted cycloalkyl, or substituted or unsubstituted heterocyclyl. In some embodiments, R₁ is phenyl or substituted phenyl, pyridyl or substituted pyridyl, pyrimidyl or substituted pyrimidyl, cyclohexyl or substituted cyclohexyl, indolyl or substituted indolyl, substituted or unsubstituted pyrrolopyridinyl, or a substituted or unsubstituted indazolyl.

In some embodiments of formulae (I), (II) and (III), R₁ is unsubstituted phenyl. In some embodiments, R₁ is substituted phenyl. In some embodiments, R₁ is phenyl substituted with two or more substituents. In some embodiments, R₁ is phenyl substituted with two or more substituents selected from the group consisting of hydroxyl, halo, perhaloalkyl, C₁-C₆ alkoxy, phenoxy, aminoacyl, substituted or unsubstituted amino, substituted or unsubstituted C₁-C₆ alkyl, cyano, and allyloxy. In some embodiments, R₁ is phenyl substituted with two or more substituents selected from halo and hydroxyl. In some embodiments, R₁ is phenyl substituted with two or more substituents selected from halo and allyloxy. In some embodiments, R₁ is phenyl substituted with one halo and one hydroxyl. In some embodiments, R₁ is phenyl substituted with one halo and one allyloxy. In some embodiments, R₁ is phenyl substituted with one halo and one C₁-C₆ alkoxy.

In some embodiments of formulae (I), (II) and (III), R₁ is a substituted phenyl moiety comprising a phenyl fused to a substituted or unsubstituted cycloalkyl or substituted or unsubstituted heterocyclyl. In some embodiments, the substituted phenyl moiety is a benzo[d][1,3]dioxolyl, a 2,3-dihydrobenzo[d][1,4]dioxinyl, a 3,4-dihydro-2H-benzo[b][1,4]dioxepinyl, a 2,3-dihydrobenzo[d]oxazolyl, a 3,4-dihydro-2H-benzo[b][1,4]oxazinyl, a 2,3,4,5-tetrahydrobenzo[b][1,4]oxazepinyl, a 2,3-dihydrobenzo[d]imidazolyl, a 1,2,3,4-tetrahydroquinoxalinyl, or a 2,3,4,5-tetrahydro-1H-benzo[b][1,4]diazepinyl. In these embodiments, the bond to R₁ can be attached at any available position on the substituted phenyl moiety.

In some embodiments, of formulae (I), (II) and (III), R₁ is a substituted or unsubstituted heteroaryl. In some embodiments, the heteroaryl is monocyclic. In some embodiments, the heteroaryl is bicyclic. In some embodiments, the heteroaryl comprises one annular heteroatom such as nitrogen, oxygen or sulfur. In some embodiments, the heteroaryl comprises two annular heteroatoms chosen from nitrogen, oxygen and sulfur. In some embodiments, the heteroaryl comprises three annular heteroatoms chosen from nitrogen, oxygen and sulfur. In these embodiments, the bond to R₁ can be attached at any available position on the heteroaryl.

In some embodiments of formulae (I), (II) and (III), R₁ is a substituted or unsubstituted pyridin-2-yl, substituted or unsubstituted pyridin-3-yl, or substituted or unsubstituted pyridin-4-yl. In some embodiments, the substituent is selected from the group consisting of C₁-C₆ alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted C₁-C₆ alkyl and substituted or unsubstituted cycloalkyl.

In some embodiments of formulae (I), (II) and (III), R₁ is a substituted or unsubstituted benzimidazol-2-yl, substituted or unsubstituted benzimidazol-4-yl, or substituted or unsubstituted benzimidazol-5-yl.

In some embodiments of formulae (I), (II) and (III), R₁ is a substituted or unsubstituted pyrazol-3-yl, or substituted or unsubstituted pyrazol-4-yl.

In some embodiments of formulae (I), (II) and (III), R₁ is a substituted or unsubstituted indol-1-yl, indol-2-yl, indol-3-yl, indol-4-yl, indol-5-yl, indol-6-yl, or indol-7-yl. In some embodiments, R₁ is a substituted or unsubstituted indol-1-yl, indol-2-yl, or indol-6-yl. In some embodiments, R₁ is a substituted indol-1-yl, substituted indol-2-yl, or substituted indol-6-yl.

In some embodiments of formulae (I), (II) and (III), R₁ is a substituted or unsubstituted pyrrolopyridin-2-yl, substituted or unsubstituted pyrrolopyridin-3-yl, substituted or unsubstituted pyrrolopyridin-4-yl, substituted or unsubstituted pyrrolopyridin-5-yl, or substituted or unsubstituted pyrrolopyridin-6-yl. In some embodiments, R₁ is substituted or unsubstituted pyrrolopyridin-2-yl or substituted or unsubstituted pyrrolopyridin-5-yl. In some embodiments, R₁ is substituted pyrrolopyridin-5-yl.

In some embodiments of formulae (I), (II) and (III), R₁ is selected from the group consisting of substituted or unsubstituted indazol-3-yl, substituted or unsubstituted indazol-4-yl, substituted or unsubstituted indazol-5-yl, substituted or unsubstituted indazol-6-yl, or substituted or unsubstituted indazol-7-yl. In some embodiments, R₁ is substituted indazol-5-yl.

In some embodiments of formulae (I), (II) and (III), R₁ is substituted or unsubstituted pyrimidin-2-yl, substituted or unsubstituted pyrimidin-4-yl or substituted or unsubstituted pyrimidin-5-yl.

In some embodiments of formulae (I), (II) and (III), R₂ and R₃ are each H. In some embodiments, R₂ is H and R₃ is C₁-C₆ alkyl. In some embodiments, R₂ is H and R₃ is methyl. In some embodiments, each R₂ and R₃ is methyl.

In some embodiments of formulae (I), (II) and (III), Y is CH. In some embodiments, Y is N.

In some embodiments of formulae (I) and (II), Z is N. In some embodiments, Z is CR₉. In some of these embodiments, R₉ is H. In some of these embodiments, R₉ is halo. In some of these embodiments, R₉ is CN. In some of these embodiments, R₉ is CF₃. In some of these embodiments, R₉ is —C(═O)NH₂. In some of these embodiments, R₉ is substituted or unsubstituted C₁-C₆ alkyl. In some embodiments, Y is CH and Z is N. In some embodiments, Y is N and Z is N. In some embodiments, Y is CH and Z is CR₉. In some embodiments, Y is N and Z is CR₉.

In some embodiments of formulae (I), (II) and (III), X is NR₅, wherein R₅ is

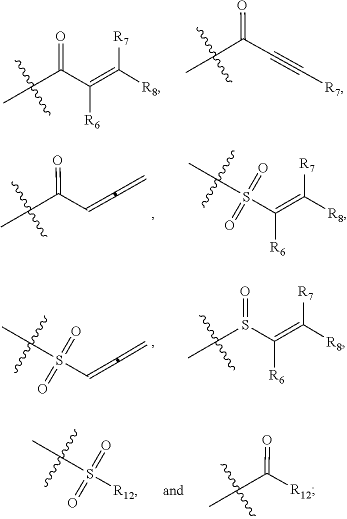

In some embodiments, X is NR₅, wherein R₅ is

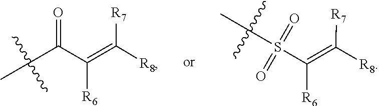

In some embodiments, X is NR₅, wherein R₅ is

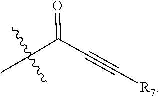

In some embodiments, X is NR₅, wherein R₅ is

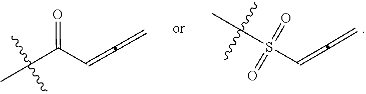

In some of these embodiments, R₆ is H. In some embodiments, R₆ is CN, halo, —NO₂, CF₃, —SO₂CH₃, —SO₂NH₂, —C(═O)R₁₂, —C(═O)OR₁₂, or —C(═O)NH₂. In some embodiments, R₆ is CN.

In some embodiments of formulae (I), (II) and (III), X is NR₅, wherein R₅ is

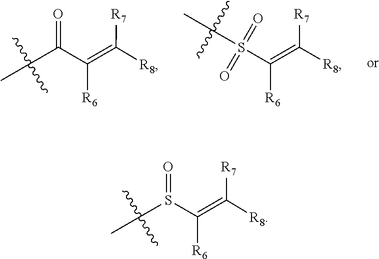

In some of these embodiments, R₆ is H. In some of these embodiments, R₆ is substituted or unsubstituted C₁-C₆ alkyl. In some of these embodiments, R₆ is substituted or unsubstituted cycloalkyl. In some of these embodiments, R₆ is substituted or unsubstituted heterocyclyl. In some of these embodiments, R₆ is substituted or unsubstituted amino. In some embodiments, R₆ is CN, halo, —NO₂, CF₃, —SO₂CH₃, —SO₂NH₂, —C(═O)R₁₂, —C(═O)OR₁₂, or —C(═O)NH₂. In some embodiments, R₆ is CN. In some of these embodiments, each R₇ and R₈ is H. In some of these embodiments, one of R₇ and R₈ is H, and the other of R₇ and R₈ is substituted or unsubstituted C₁-C₆ alkyl. In some of these embodiments, R₇ is H, and R₈ is substituted or unsubstituted C₁-C₆ alkyl. In some of these embodiments, R₇ is H, and R₈ is substituted or unsubstituted cycloalkyl. In some of these embodiments, R₇ is H, and R₈ is substituted or unsubstituted heterocyclyl. In some of these embodiments, R₇ is H, and R₈ is substituted or unsubstituted amino.

In some embodiments of formulae (I), (II) and (III), X is NR₅, m is 0, and the indoline moiety is attached to the heterocycle as an indolin-2-yl group. In some embodiments, X is NR₅, m is 0, and the indoline moiety is attached to the heterocycle as an indolin-3-yl group. In some embodiments, X is NR₅, m is 1, and the dihydroquinoline moiety is attached to the heterocycle as a dihydroquinolin-2-yl group. In some embodiments, X is NR₅, m is 1, and the dihydroquinoline moiety is attached to the heterocycle as a dihydroquinolin-3-yl group. In some embodiments, X is NR₅, m is 1, and the dihydroquinoline moiety is attached to the heterocycle as a dihydroquinolin-4-yl group.

In some embodiments of formulae (I), (II) and (III), X is CH₂, m is 0, and the dihydroindene moiety is attached to the heterocycle as a dihydroinden-1-yl group. In some embodiments, X is CH₂, m is 0, and the dihydroindene moiety is attached to the heterocycle as a dihydroinden-2-yl group. In some embodiments, X is CH₂, m is 1, and a tetrahydronaphthalene moiety is attached to the heterocycle as a tetrahydronaphthalen-1-yl group. In some embodiments, X is CH₂, m is 1, and the tetrahydronaphthalene moiety is attached to the heterocycle as a tetrahydronaphthalen-2-yl group.

In some embodiments of formulae (I), (II) and (III), n is 1 or 2, and each R₄ is independently hydroxyl, halo, C₁-C₈ perhaloalkyl, substituted or unsubstituted C₁-C₈ alkyl, acyl, substituted or unsubstituted amino, acylamino, aminoacyl, —R₅ or —NHR₅. In some embodiments, R₄ is hydroxyl. In some embodiments, R₄ is fluoro, chloro or bromo. In some embodiments, R₄ is —R₅ or —NHR₅.

In some embodiments of formulae (I), (II) and (III), the compound is of structure A-1, A-2, A-3, A-4, A-5, A-6, A-7, A-8, A-9, or A-10:

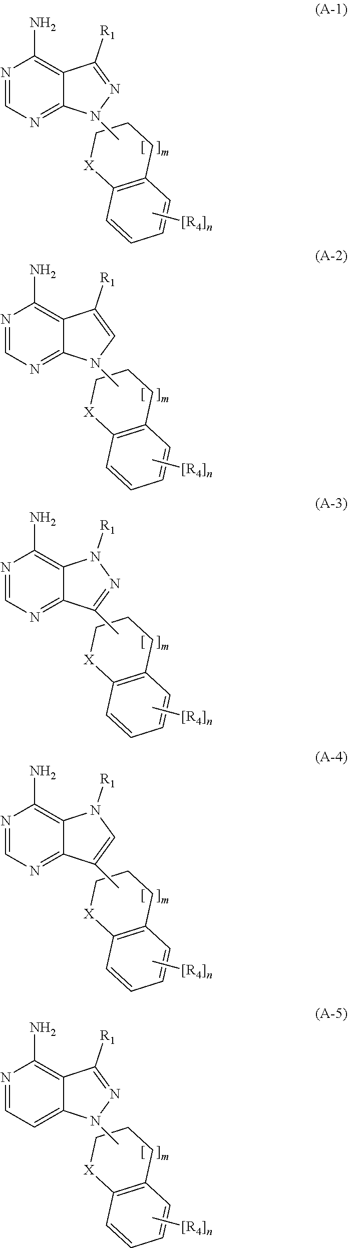

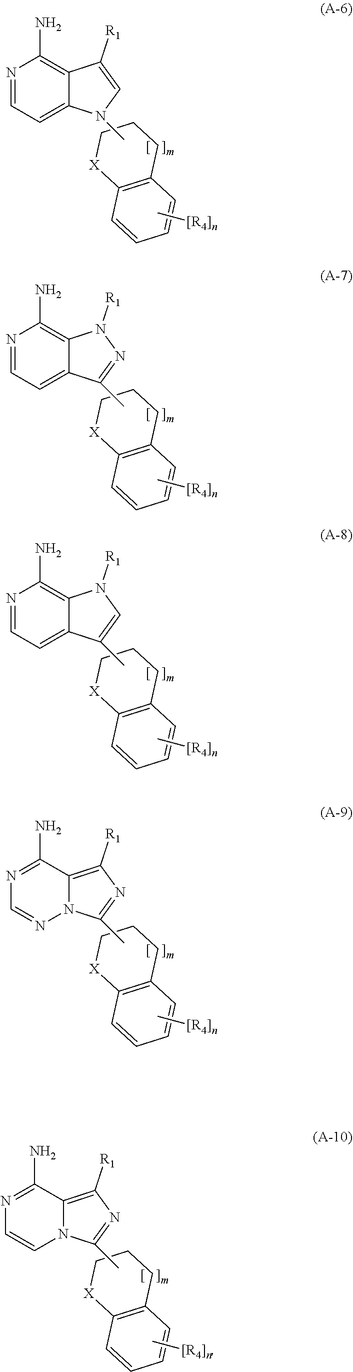

or a salt thereof; wherein R₁, X, m, n, R₄, are as described for formulae (I), (II) and (III).

In some embodiments of formulae (I), (II) and (III), the compound is of structure A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, or A-10e:

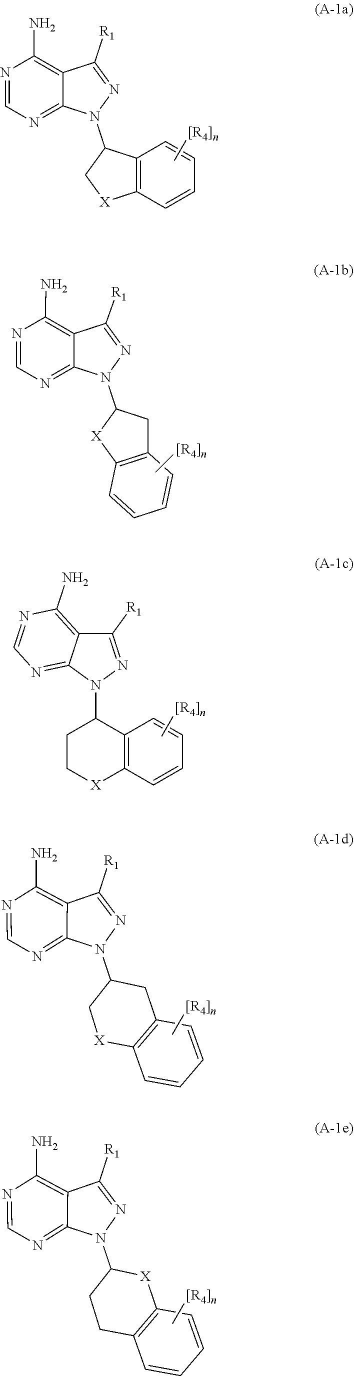

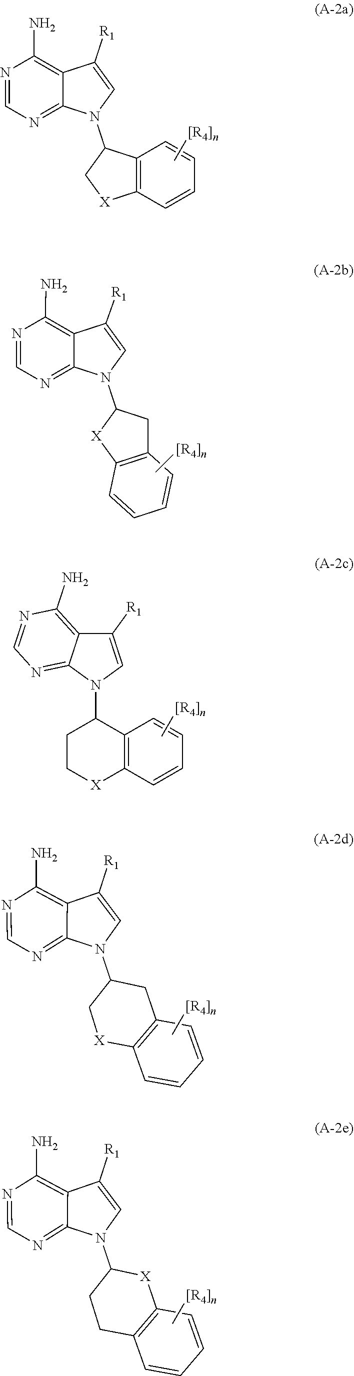

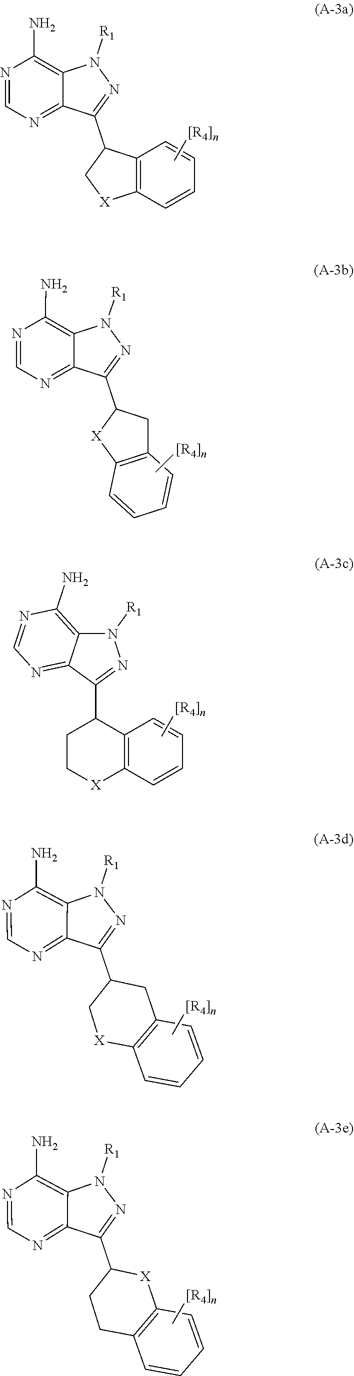

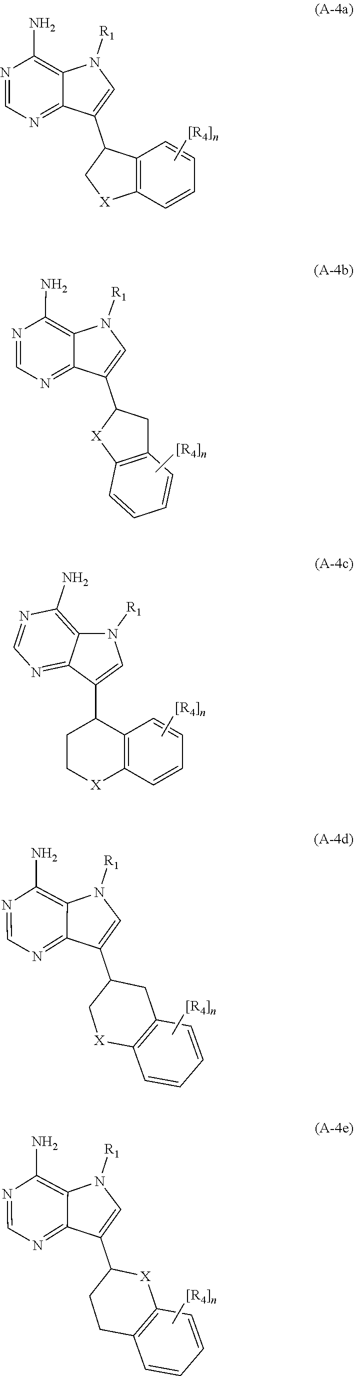

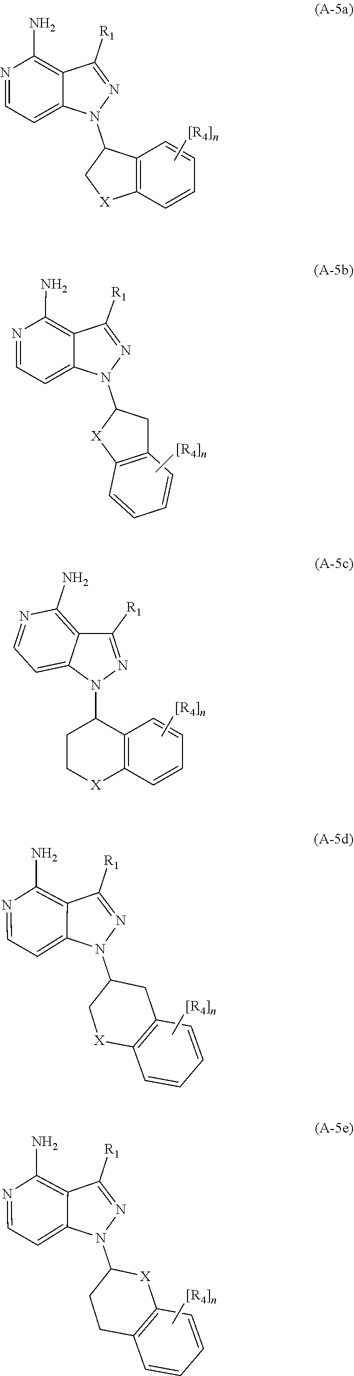

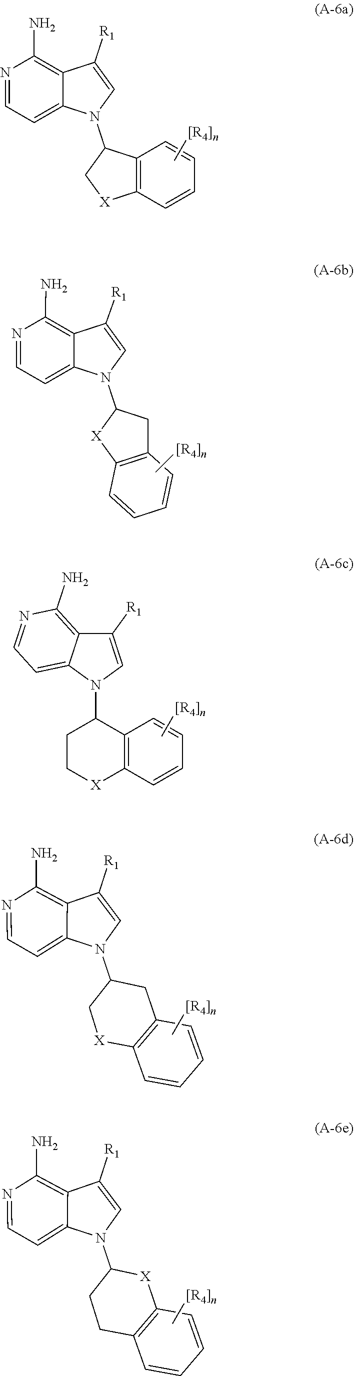

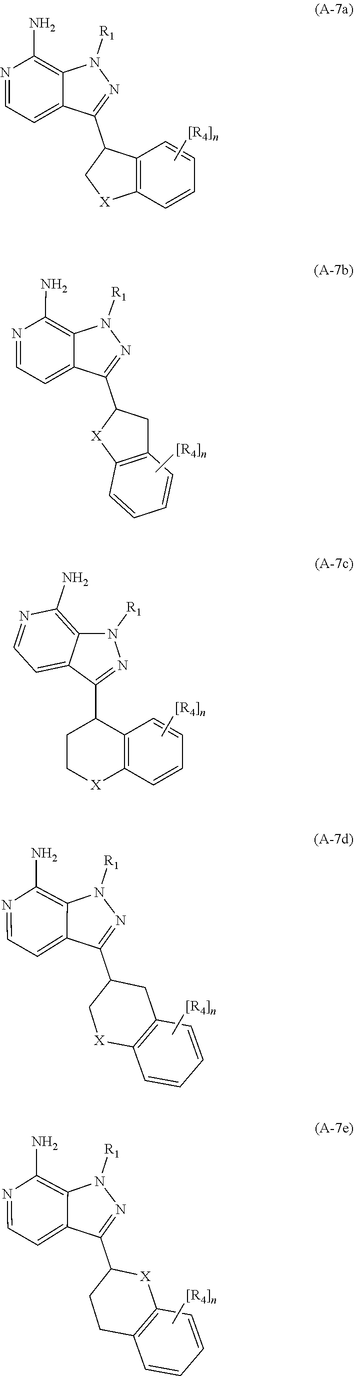

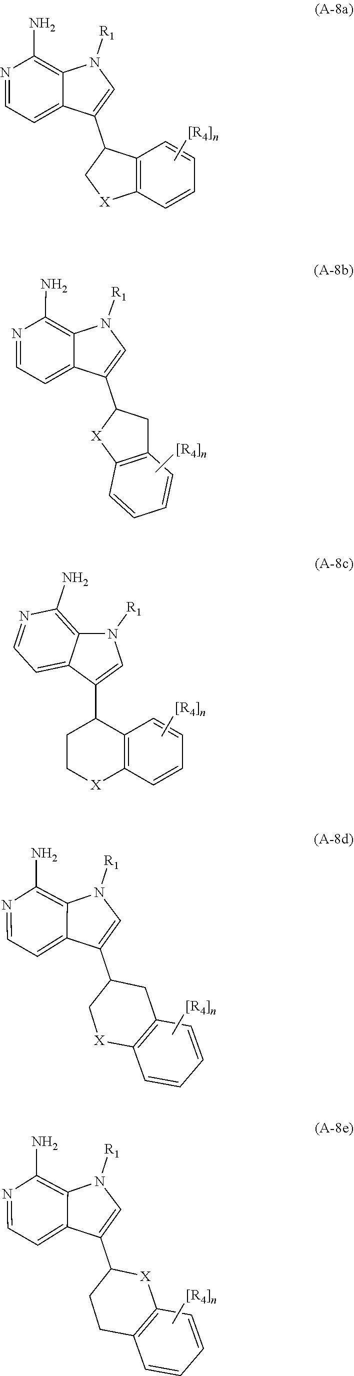

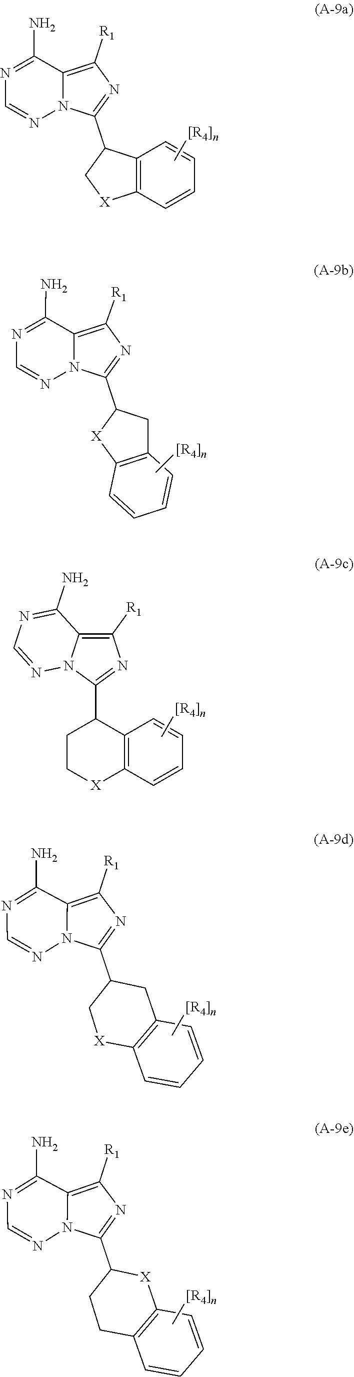

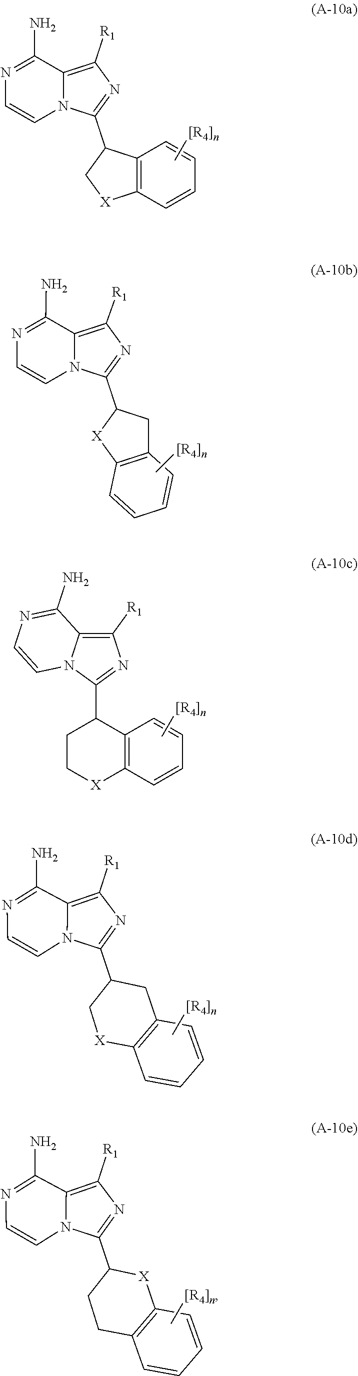

or a salt thereof; wherein R₁, R₄, X, n, R₅, R₆, R₇ and R₈, where present, are as described for formulae A-1, A-2, A-3, A-4, A-5, A-6, A-7, A-8, A-9 and A-10.

In some embodiments of formulae A-1a to A-10e, R₁ is substituted or unsubstituted C₁-C₆ alkyl. In some embodiments, R₁ is substituted or unsubstituted C₂-C₆ alkenyl. In some embodiments, R₁ is substituted or unsubstituted aryl. In some embodiments, R₁ is substituted or unsubstituted cycloalkyl. In some embodiments, R₁ is substituted or unsubstituted heterocyclyl. In some embodiments, R₁ is substituted or unsubstituted heteroaryl. In some embodiments, R₁ is substituted or unsubstituted phenyl. In some embodiments, R₁ is substituted or unsubstituted pyridyl. In some embodiments, R₁ is substituted or unsubstituted pyrazolyl. In some embodiments, R₁ is substituted or unsubstituted benzimidazolyl. In some embodiments, R₁ is substituted or unsubstituted pyrimidyl. In some embodiments, R₁ is substituted or unsubstituted indolyl. In some embodiments, R₁ is substituted or unsubstituted pyrrolopyridinyl. In some embodiments, R₁ is substituted or unsubstituted indazolyl.

In some embodiments of formulae A-1a to A-10e, n is 0. In some embodiments, n is 1. In some embodiments, n is 2. In some embodiments, n is 3. In some embodiments, n is 4.

In some embodiments of formulae A-1a to A-10e, X is CH₂. In some embodiments of formulae A-1a to A-10e, X is NR₅, wherein R₅ is

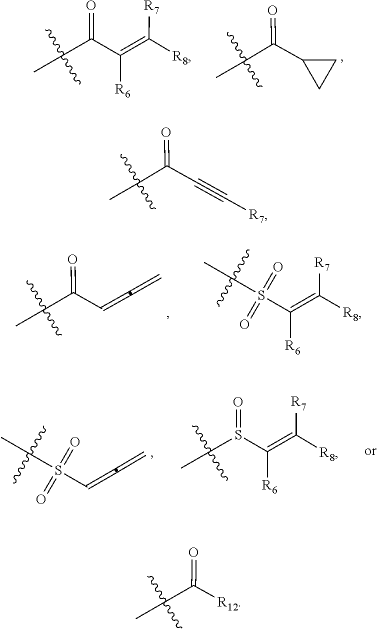

In some embodiments of formulae (I), (II) and (III), the compound is of structure A-1a-1, A-1a-2, A-1a-3, A-1a-4, A-1a-5, A-1a-6, A-1a-7, A-1a-8, A-1b-1, A-1b-2, A-1b-3, A-1b-4, A-1b-5, A-1b-6, A-1b-7, A-1b-8, A-1c-1, A-1c-2, A-1c-3, A-1c-4, A-1c-5, A-1c-6, A-1c-7, A-1c-8, A-1d-1, A-1d-2, A-1d-3, A-1d-4, A-1d-5, A-1d-6, A-1d-7, A-1d-8, A-1e-1, A-1e-2, A-1e-3, A-1e-4, A-1e-5, A-1e-6, A-1e-7, A-1e-8, A-2a-1, A-2a-2, A-2a-3, A-2a-4, A-2a-5, A-2a-6, A-2a-7, A-2a-8, A-2b-1, A-2b-2, A-2b-3, A-2b-4, A-2b-5, A-2b-6, A-2b-7, A-2b-8, A-2c-1, A-2c-2, A-2c-3, A-2c-4, A-2c-5, A-2c-6, A-2c-7, A-2c-8, A-2d-1, A-2d-2, A-2d-3, A-2d-4, A-2d-5, A-2d-6, A-2d-7, A-2d-8, A-2e-1, A-2e-2, A-2e-3, A-2e-4, A-2e-5, A-2e-6, A-2e-7, A-2e-8, A-3a-1, A-3a-2, A-3a-3, A-3a-4, A-3a-5, A-3a-6, A-3a-7, A-3a-8, A-3b-1, A-3b-2, A-3b-3, A-3b-4, A-3b-5, A-3b-6, A-3b-7, A-3b-8, A-3c-1, A-3c-2, A-3c-3, A-3c-4, A-3c-5, A-3c-6, A-3c-7, A-3c-8, A-3d-1, A-3d-2, A-3d-3, A-3d-4, A-3d-5, A-3d-6, A-3d-7, A-3d-8, A-3e-1, A-3e-2, A-3e-3, A-3e-4, A-3e-5, A-3e-6, A-3e-7, A-3e-8, A-4a-1, A-4a-2, A-4a-3, A-4a-4, A-4a-5, A-4a-6, A-4a-7, A-4a-8, A-4b-1, A-4b-2, A-4b-3, A-4b-4, A-4b-5, A-4b-6, A-4b-7, A-4b-8, A-4c-1, A-4c-2, A-4c-3, A-4c-4, A-4c-5, A-4c-6, A-4c-7, A-4c-8, A-4d-1, A-4d-2, A-4d-3, A-4d-4, A-4d-5, A-4d-6, A-4d-7, A-4d-8, A-4e-1, A-4e-2, A-4e-3, A-4e-4, A-4e-5, A-4e-6, A-4e-7, A-4e-8, A-5a-1, A-5a-2, A-5a-3, A-5a-4, A-5a-5, A-5a-6, A-5a-7, A-5a-8, A-5b-1, A-5b-2, A-5b-3, A-5b-4, A-5b-5, A-5b-6, A-5b-7, A-5b-8, A-5c-1, A-5c-2, A-5c-3, A-5c-4, A-5c-5, A-5c-6, A-5c-7, A-5c-8, A-5d-1, A-5d-2, A-5d-3, A-5d-4, A-5d-5, A-5d-6, A-5d-7, A-5d-8, A-5e-1, A-5e-2, A-5e-3, A-5e-4, A-5e-5, A-5e-6, A-5e-7, A-5e-8, A-6a-1, A-6a-2, A-6a-3, A-6a-4, A-6a-5, A-6a-6, A-6a-7, A-6a-8, A-6b-1, A-6b-2, A-6b-3, A-6b-4, A-6b-5, A-6b-6, A-6b-7, A-6b-8, A-6c-1, A-6c-2, A-6c-3, A-6c-4, A-6c-5, A-6c-6, A-6c-7, A-6c-8, A-6d-1, A-6d-2, A-6d-3, A-6d-4, A-6d-5, A-6d-6, A-6d-7, A-6d-8, A-6e-1, A-6e-2, A-6e-3, A-6e-4, A-6e-5, A-6e-6, A-6e-7, A-6e-8, A-7a-1, A-7a-2, A-7a-3, A-7a-4, A-7a-5, A-7a-6, A-7a-7, A-7a-8, A-7b-1, A-7b-2, A-7b-3, A-7b-4, A-7b-5, A-7b-6, A-7b-7, A-7b-8, A-7c-1, A-7c-2, A-7c-3, A-7c-4, A-7c-5, A-7c-6, A-7c-7, A-7c-8, A-7d-1, A-7d-2, A-7d-3, A-7d-4, A-7d-5, A-7d-6, A-7d-7, A-7d-8, A-7e-1, A-7e-2, A-7e-3, A-7e-4, A-7e-5, A-7e-6, A-7e-7, A-7e-8, A-8a-1, A-8a-2, A-8a-3, A-8a-4, A-8a-5, A-8a-6, A-8a-7, A-8a-8, A-8b-1, A-8b-2, A-8b-3, A-8b-4, A-8b-5, A-8b-6, A-8b-7, A-8b-8, A-8c-1, A-8c-2, A-8c-3, A-8c-4, A-8c-5, A-8c-6, A-8c-7, A-8c-8, A-8d-1, A-8d-2, A-8d-3, A-8d-4, A-8d-5, A-8d-6, A-8d-7, A-8d-8, A-8e-1, A-8e-2, A-8e-3, A-8e-4, A-8e-5, A-8e-6, A-8e-7, A-8e-8, A-9a-1, A-9a-2, A-9a-3, A-9a-4, A-9a-5, A-9a-6, A-9a-7, A-9a-9, A-9b-1, A-9b-2, A-9b-3, A-9b-4, A-9b-5, A-9b-6, A-9b-7, A-9b-8, A-9c-1, A-9c-2, A-9c-3, A-9c-4, A-9c-5, A-9c-6, A-9c-7, A-9c-8, A-9d-1, A-9d-2, A-9d-3, A-9d-4, A-9d-5, A-9d-6, A-9d-7, A-9d-8, A-9e-1, A-9e-2, A-9e-3, A-9e-4, A-9e-5, A-9e-6, A-9e-7, A-9e-8, A-10a-1, A-10a-2, A-10a-3, A-10a-4, A-10a-5, A-10a-6, A-10a-7, A-10A-10, A-10b-1, A-10b-2, A-10b-3, A-10b-4, A-10b-5, A-10b-6, A-10b-7, A-10b-8, A-10c-1, A-10c-2, A-10c-3, A-10c-4, A-10c-5, A-10c-6, A-10c-7, A-10c-8, A-10d-1, A-10d-2, A-10d-3, A-10d-4, A-10d-5, A-10d-6, A-10d-7, A-10d-8, A-10e-1, A-10e-2, A-10e-3, A-10e-4, A-10e-5, A-10e-6, A-10e-7, or A-10e-8:

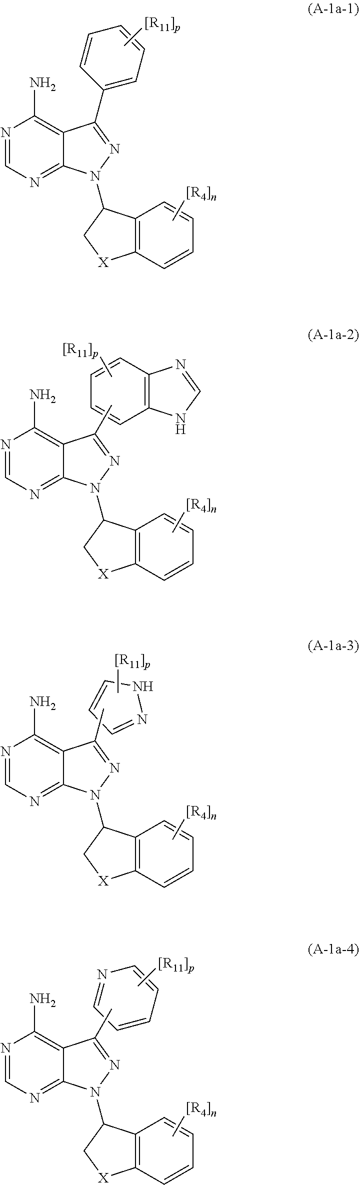

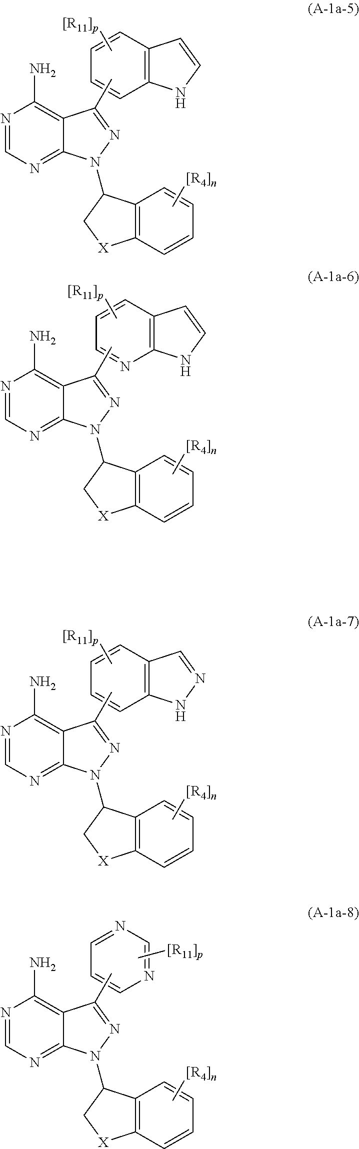

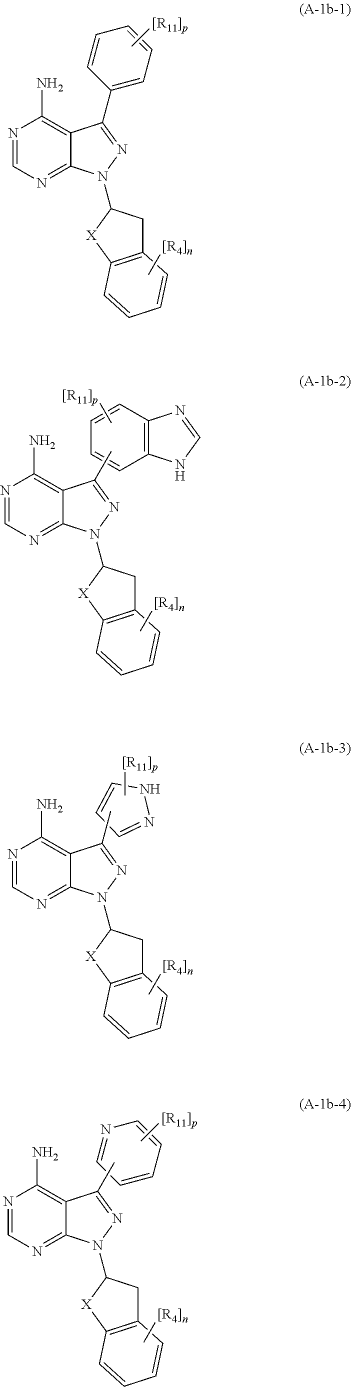

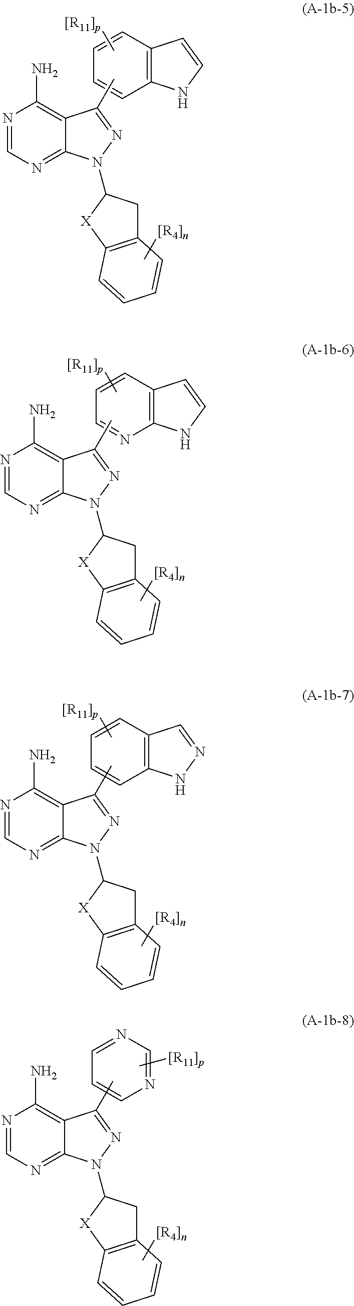

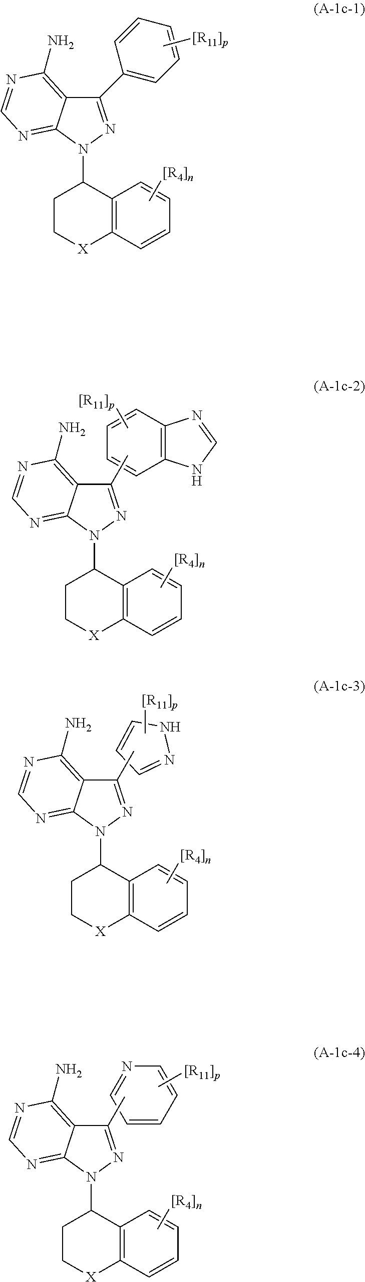

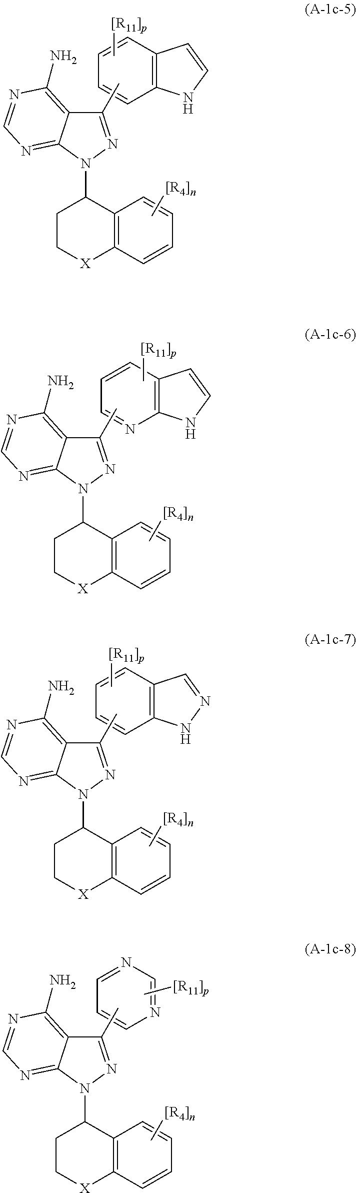

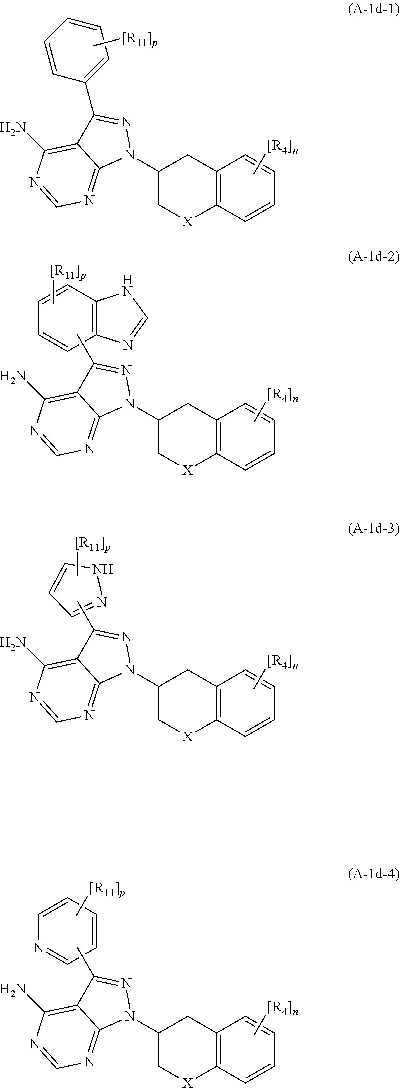

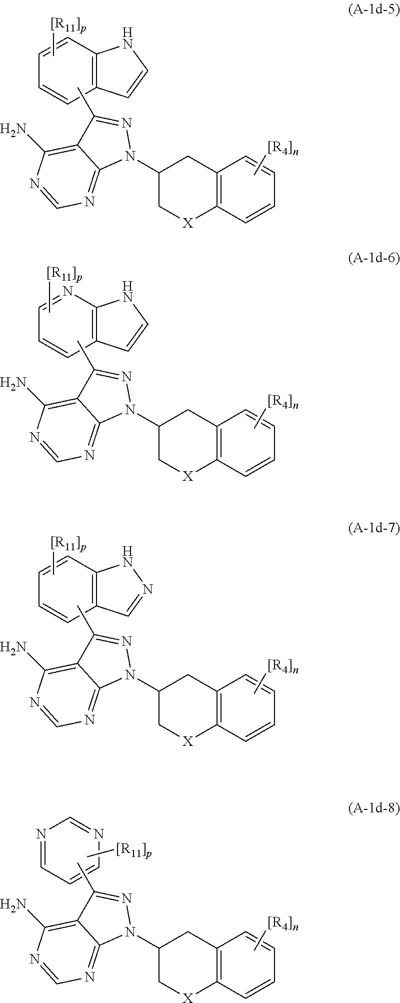

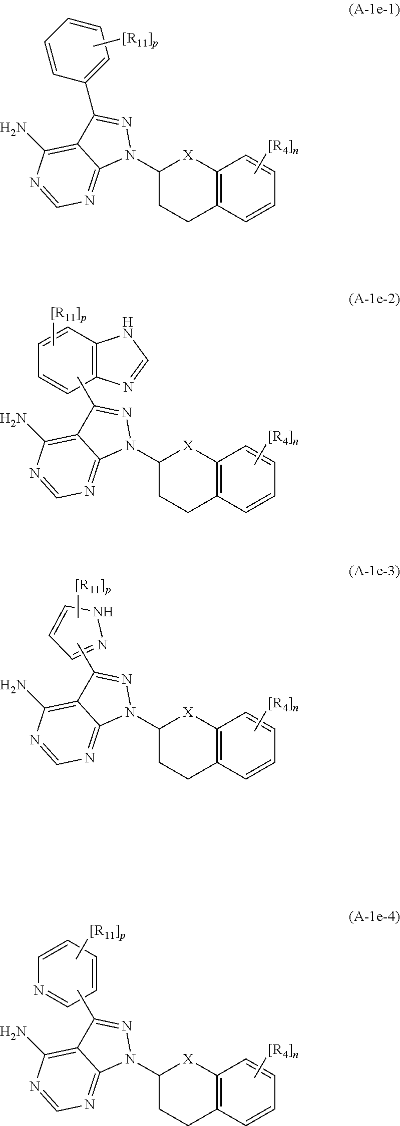

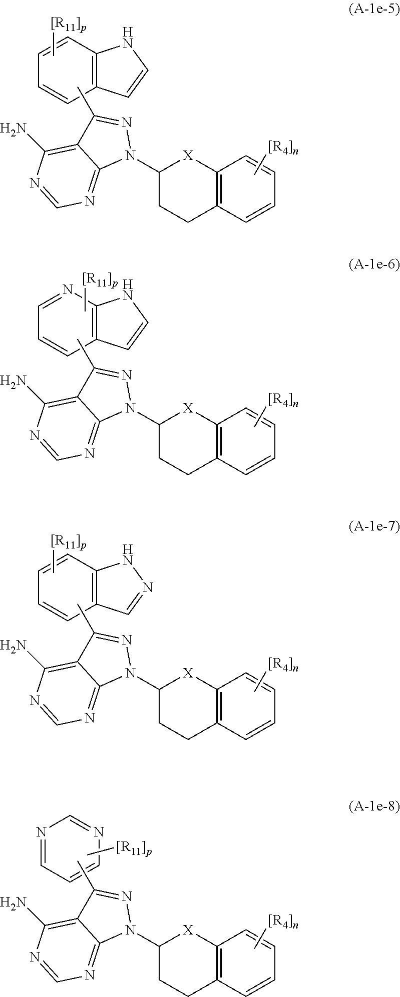

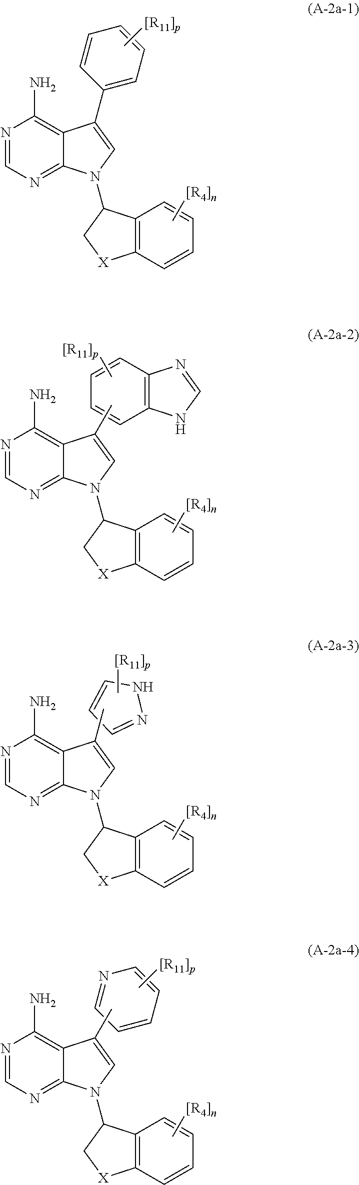

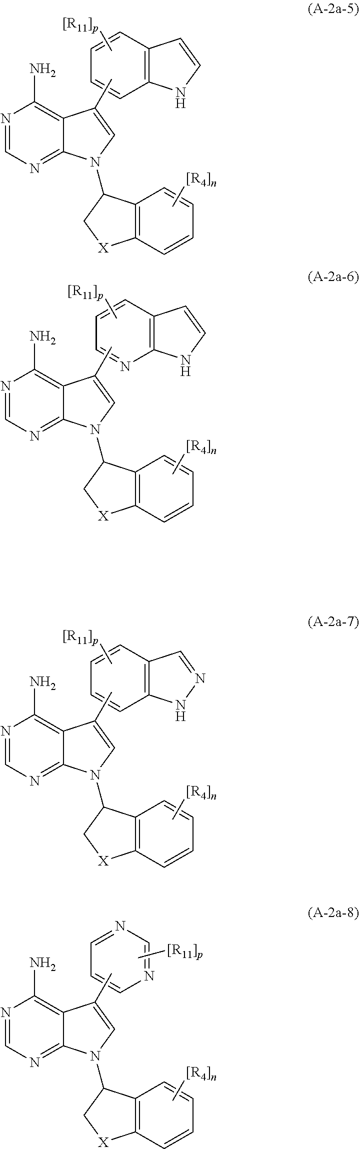

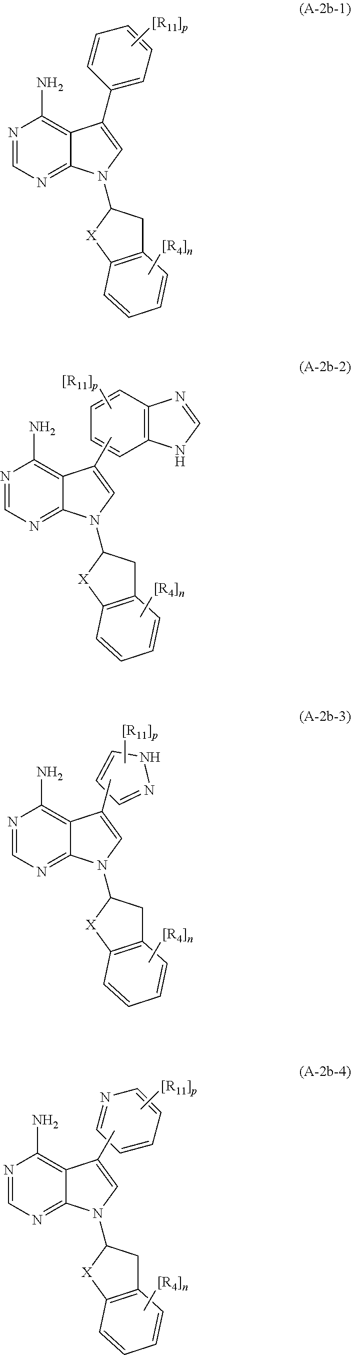

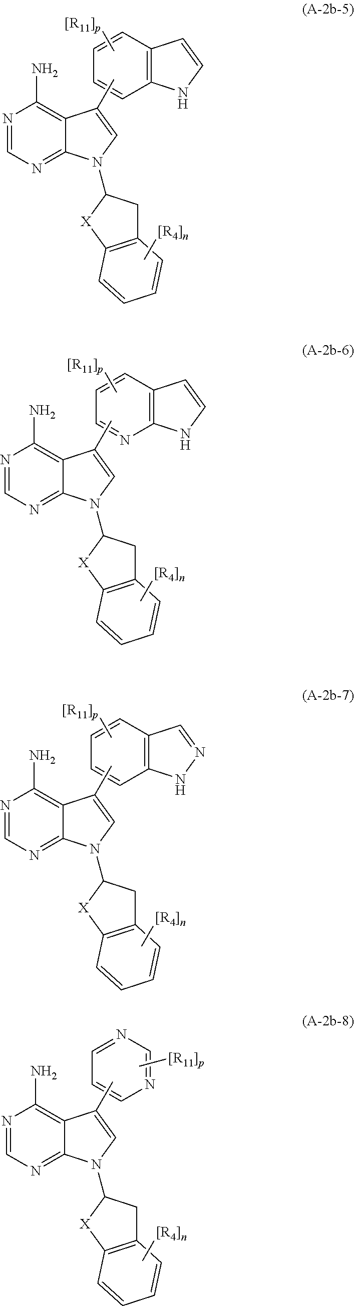

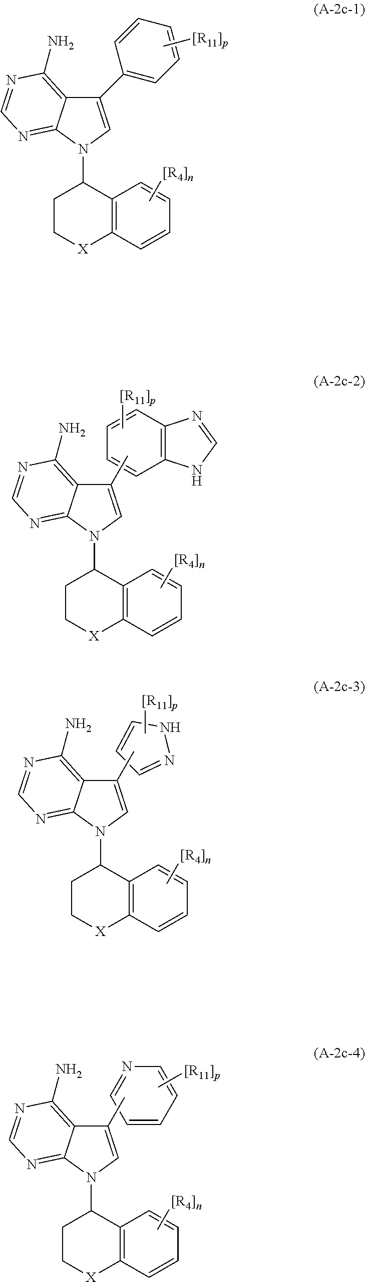

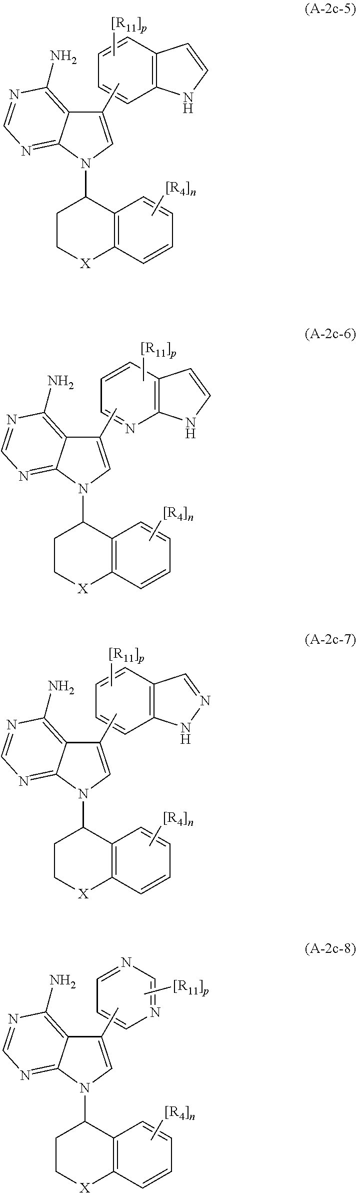

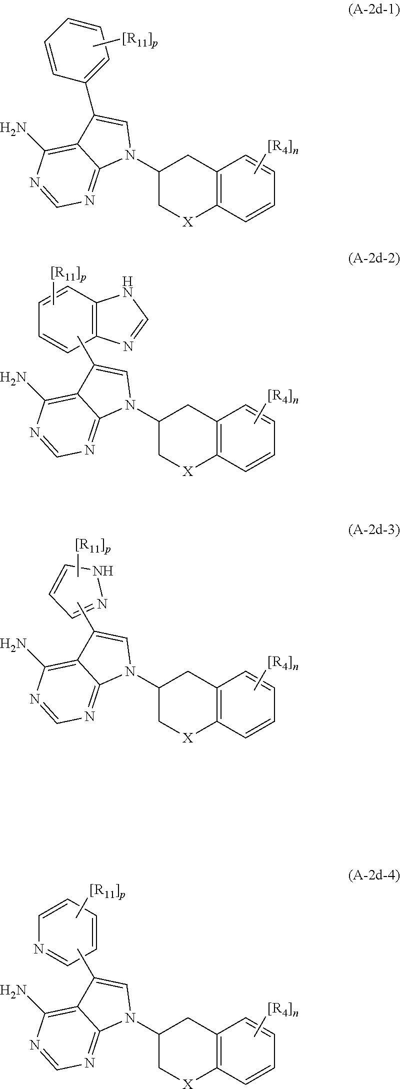

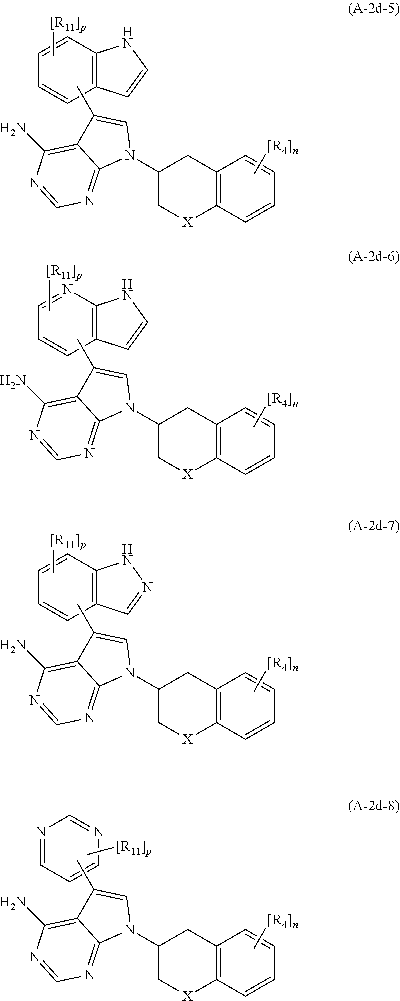

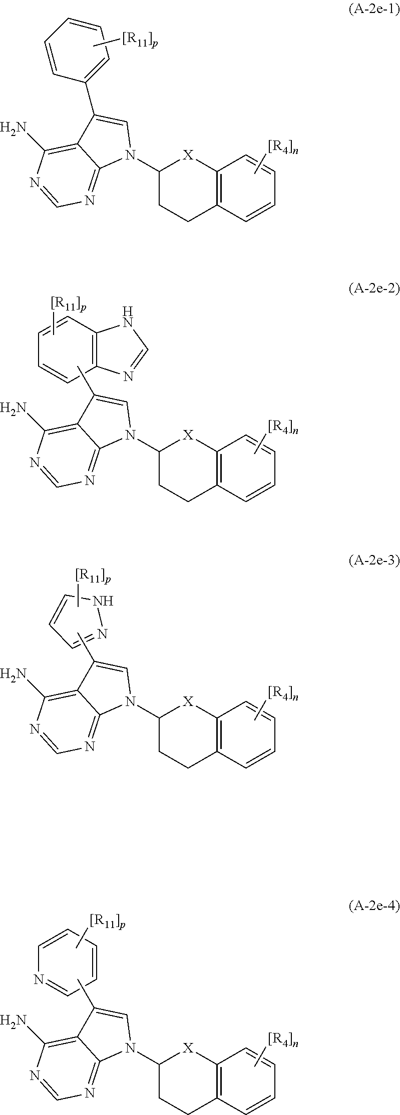

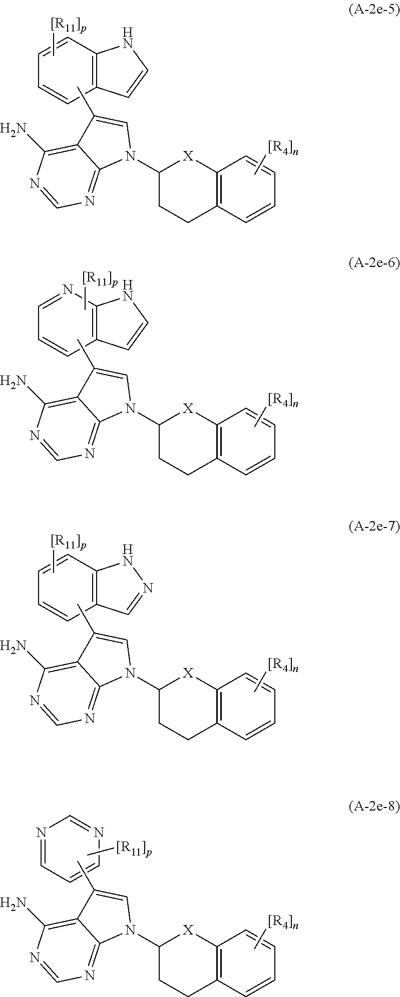

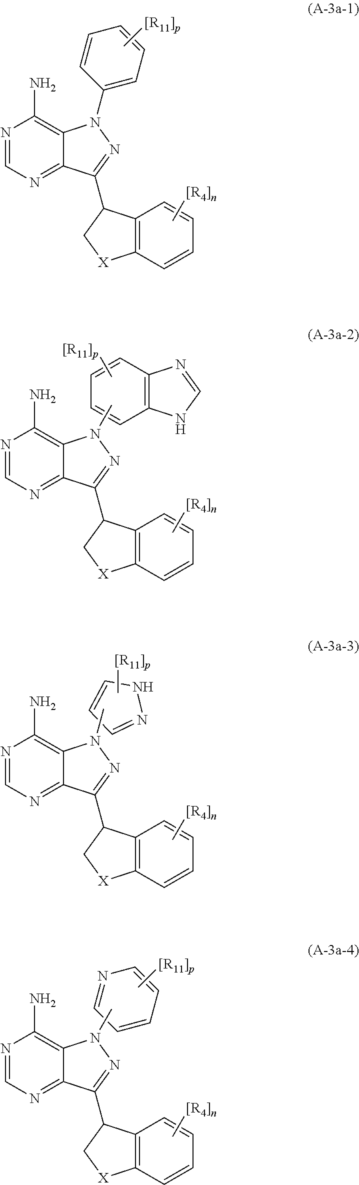

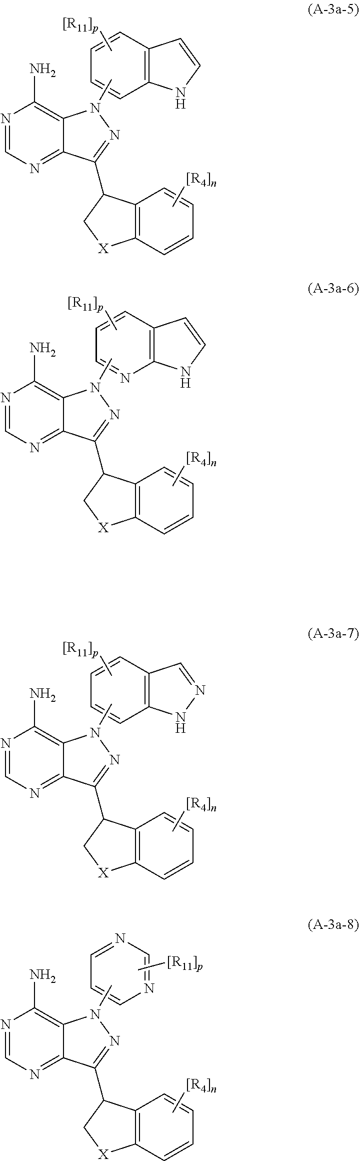

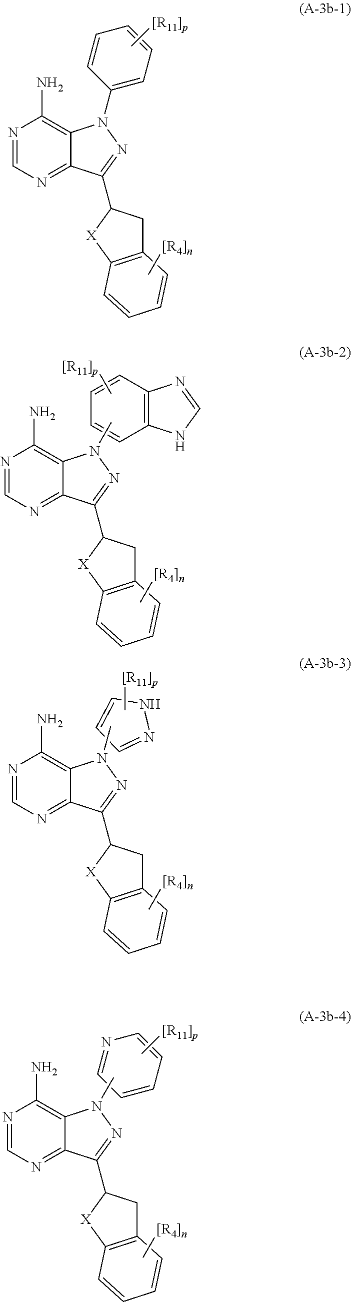

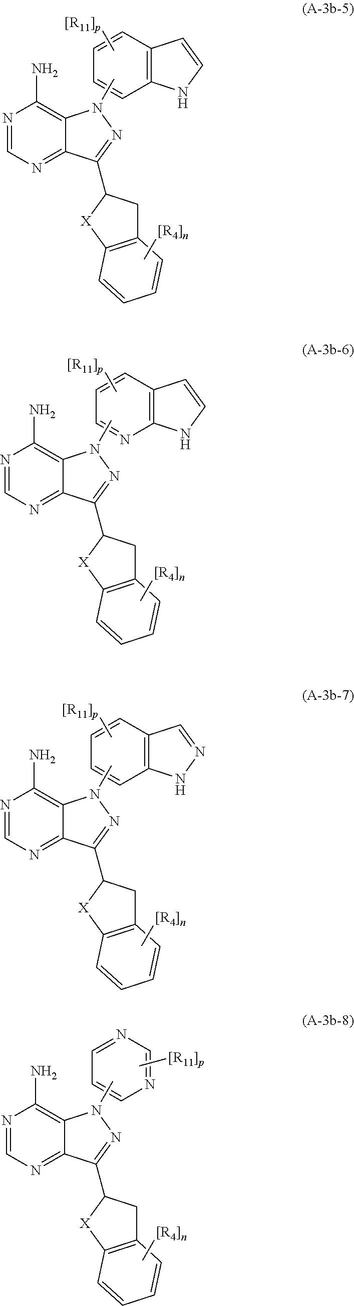

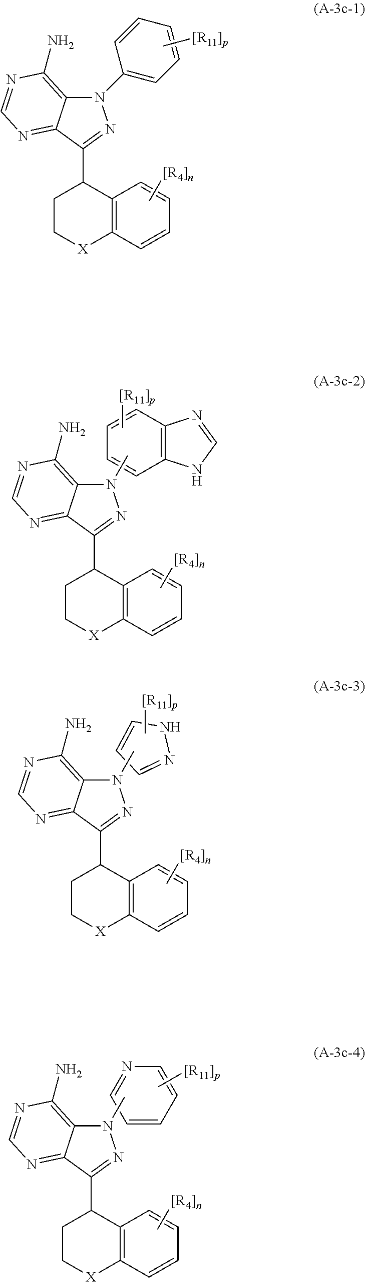

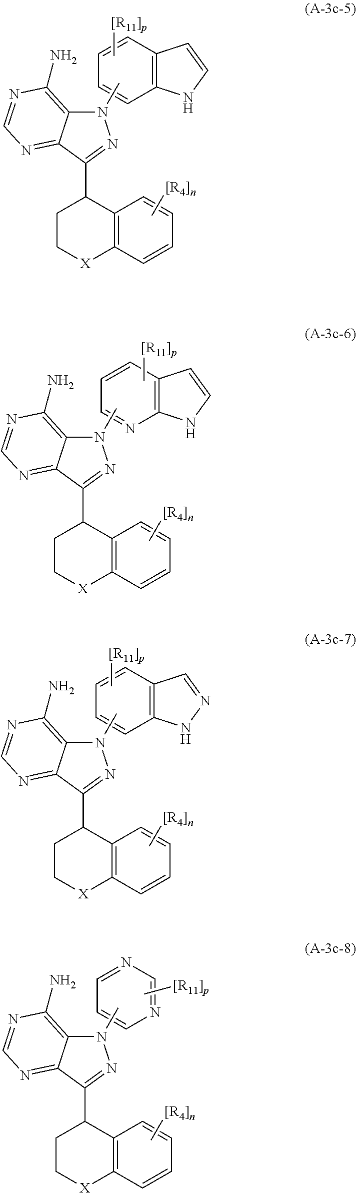

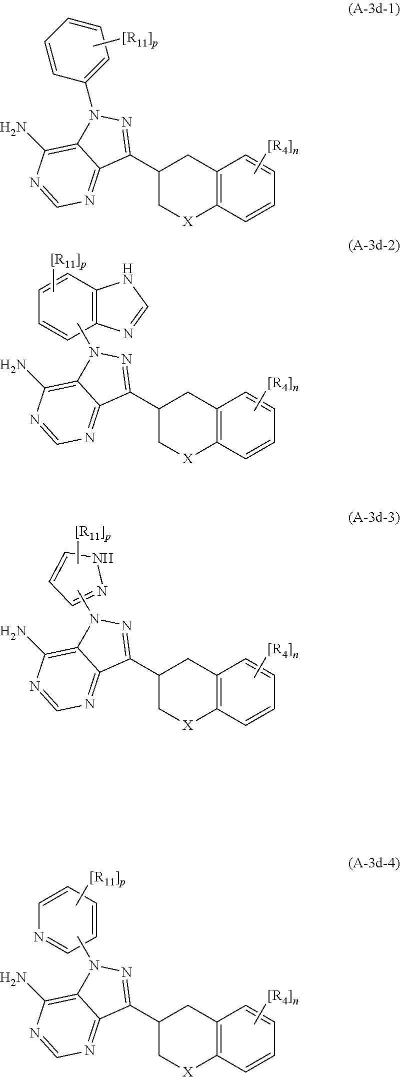

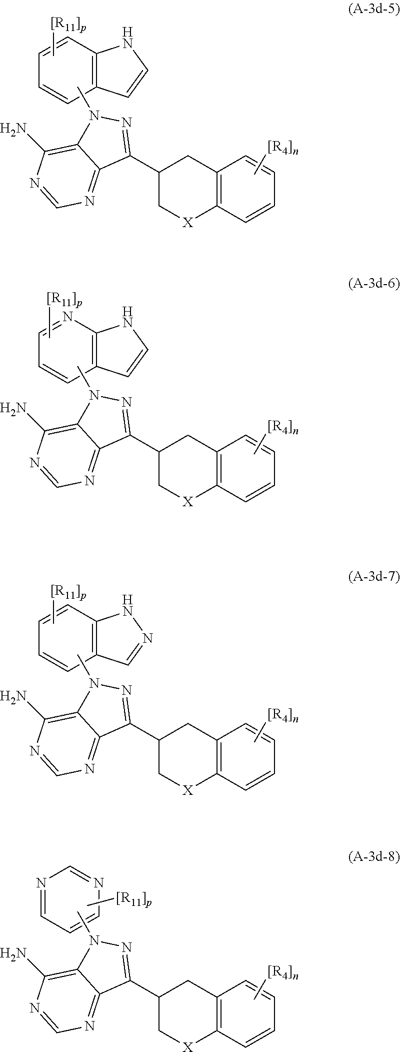

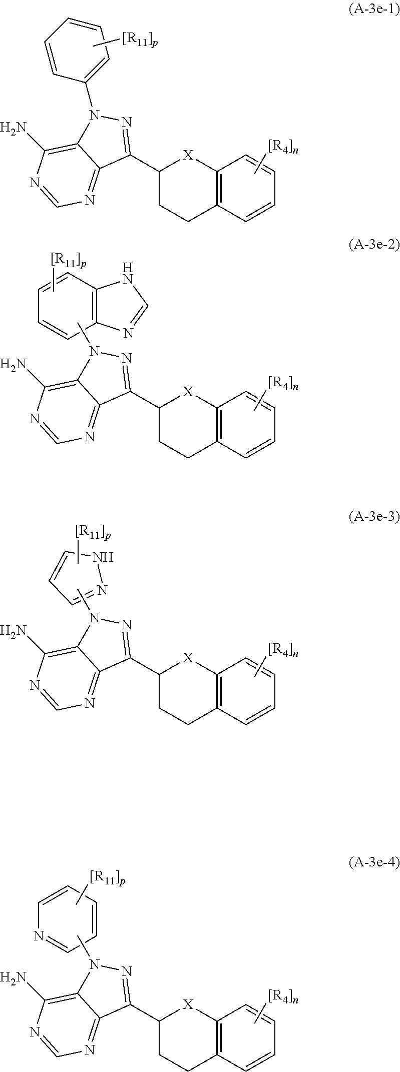

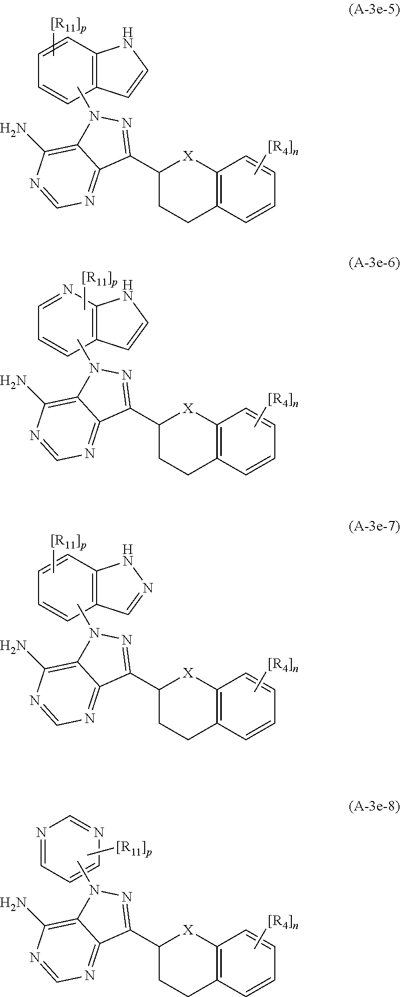

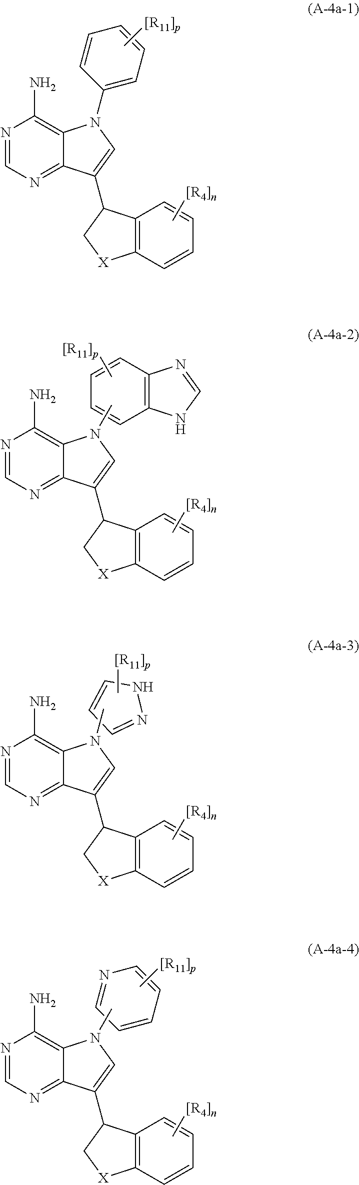

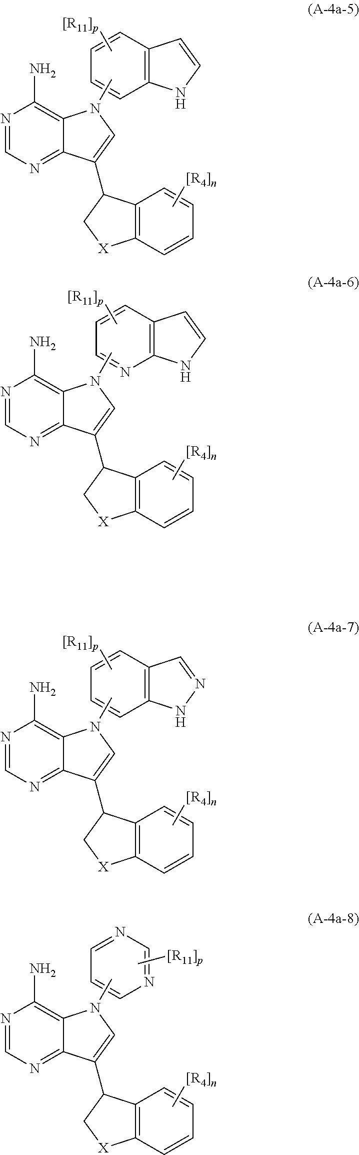

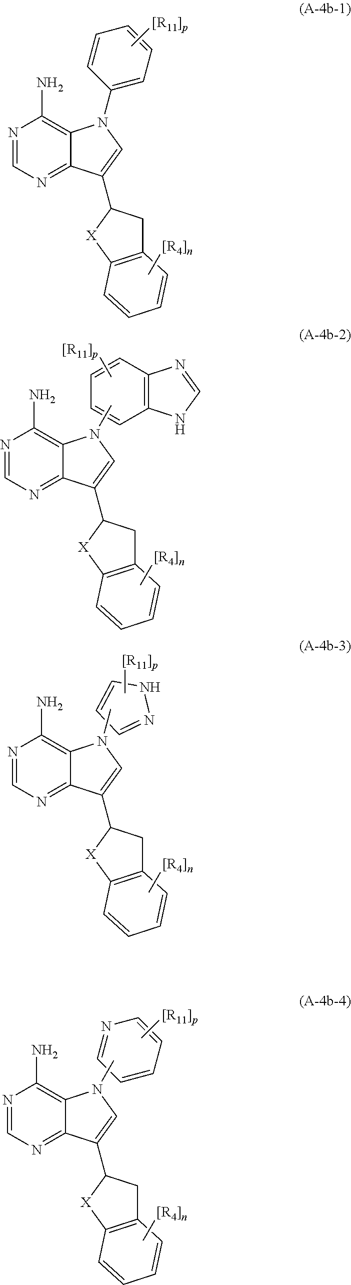

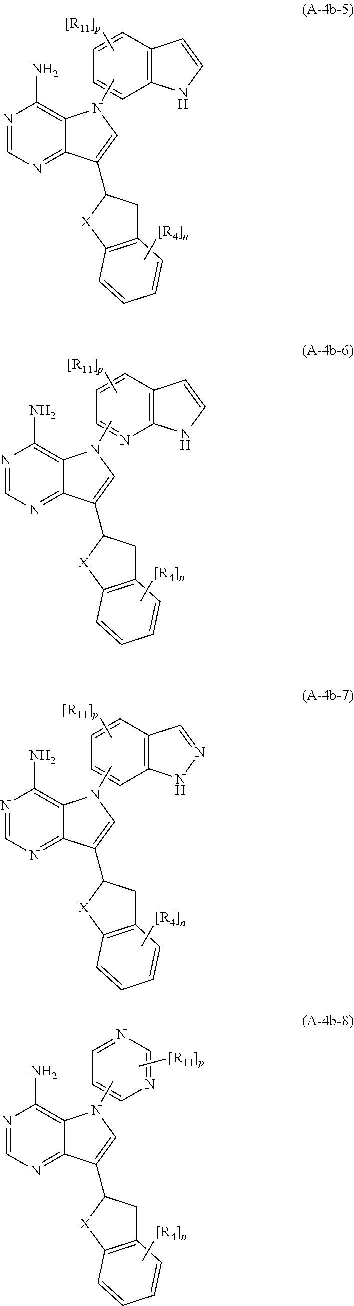

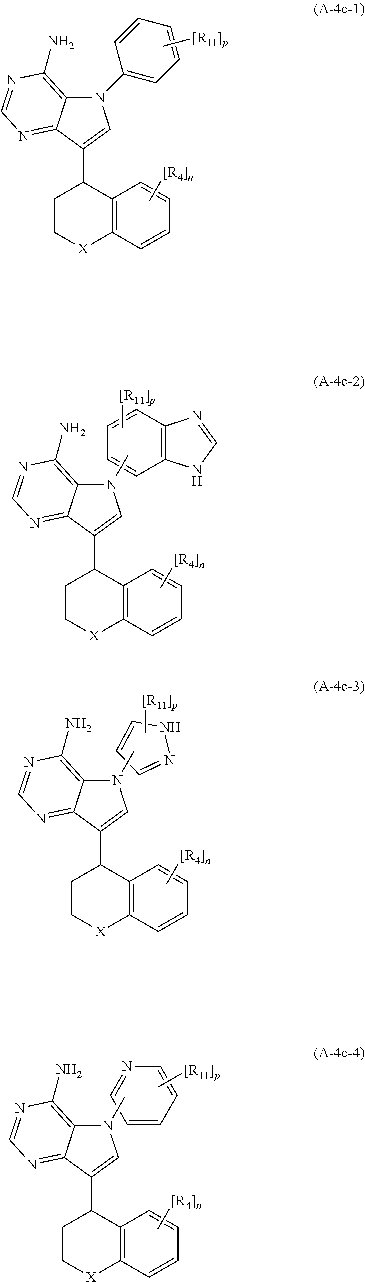

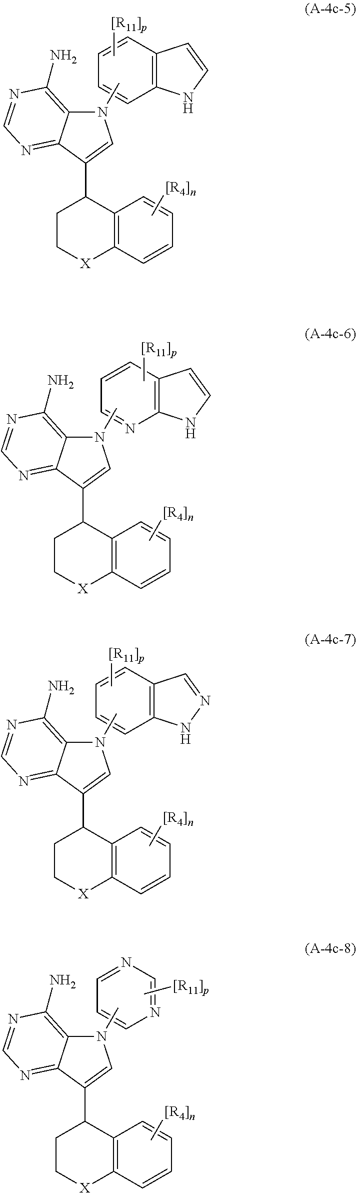

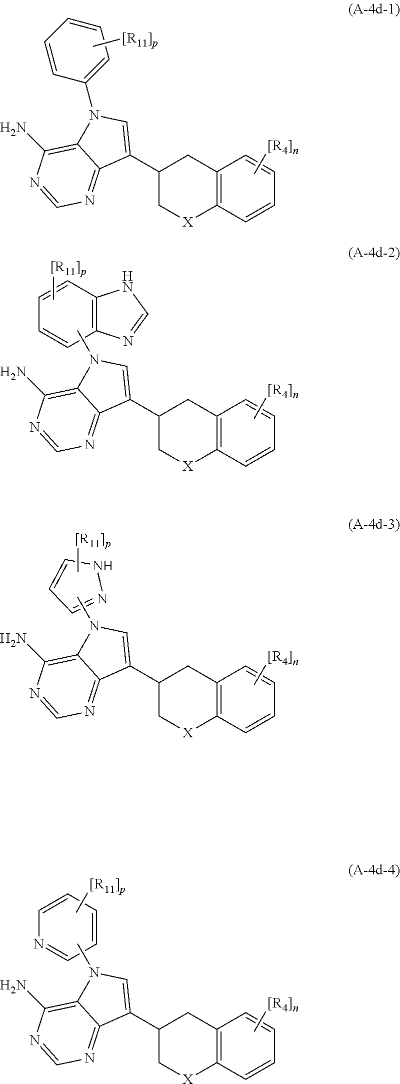

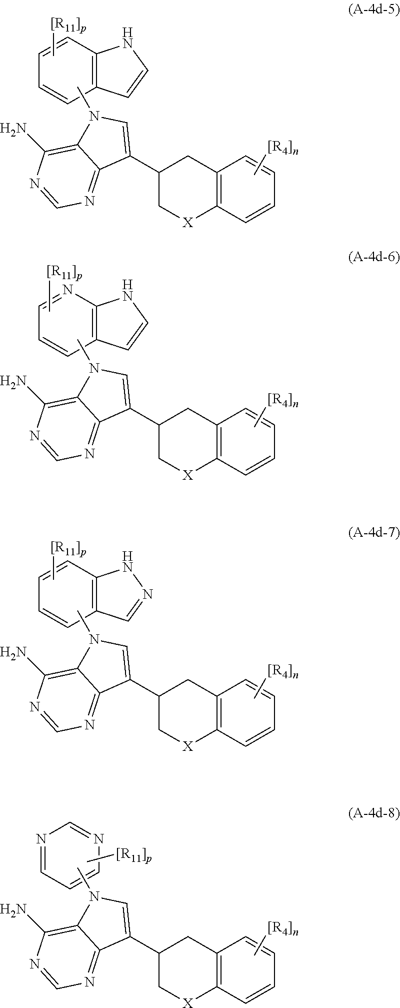

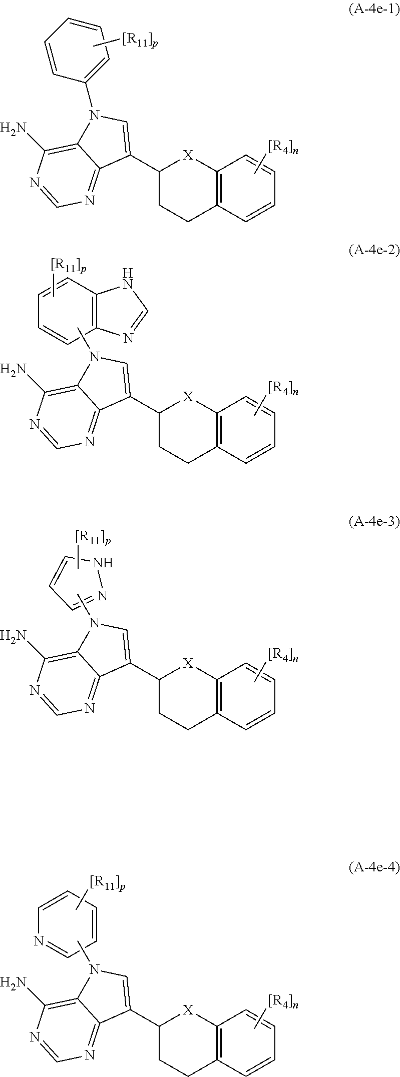

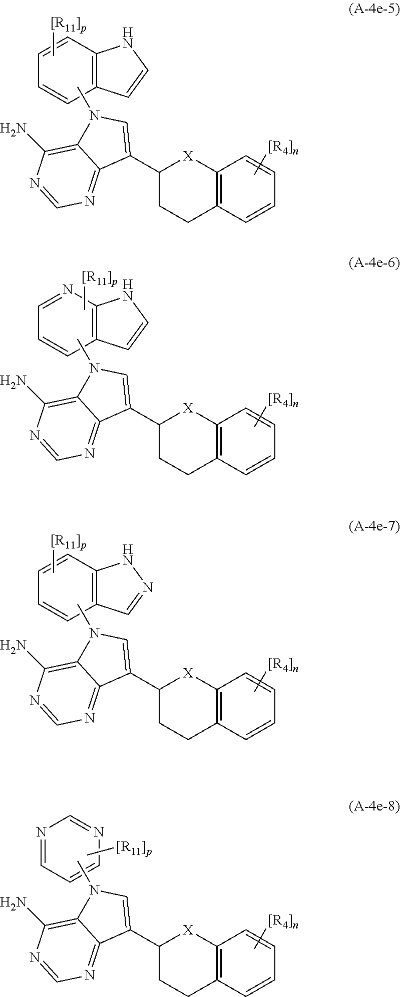

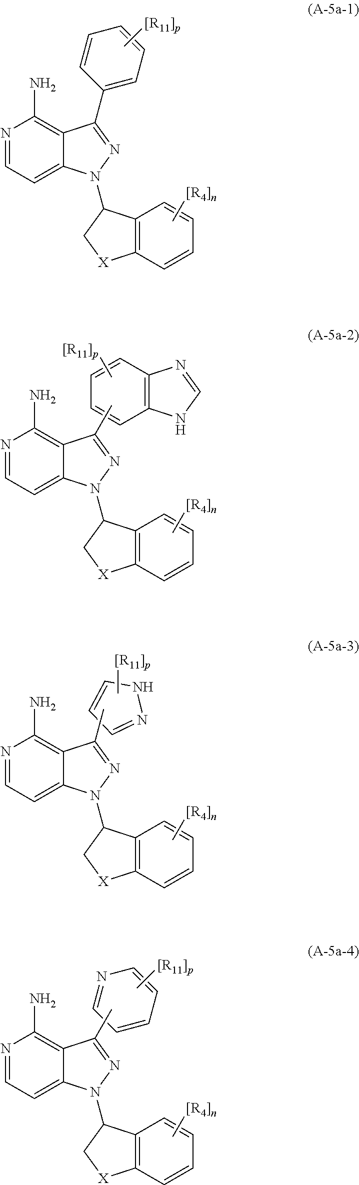

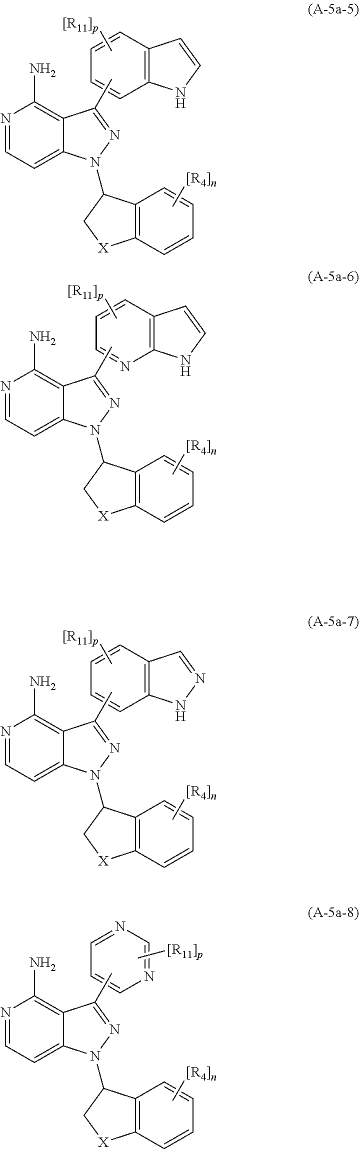

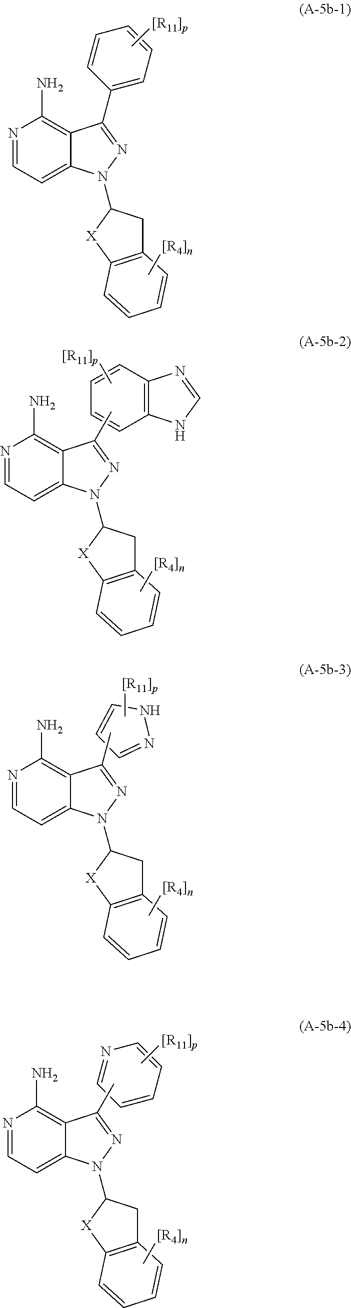

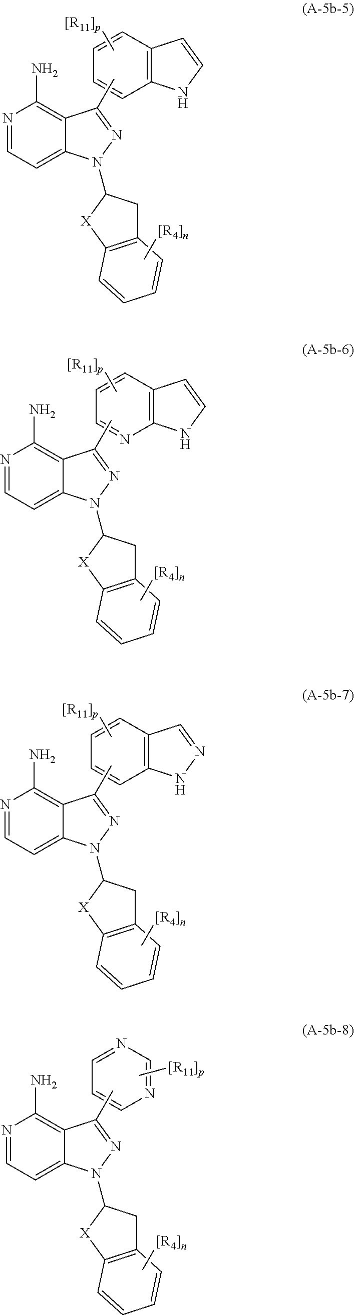

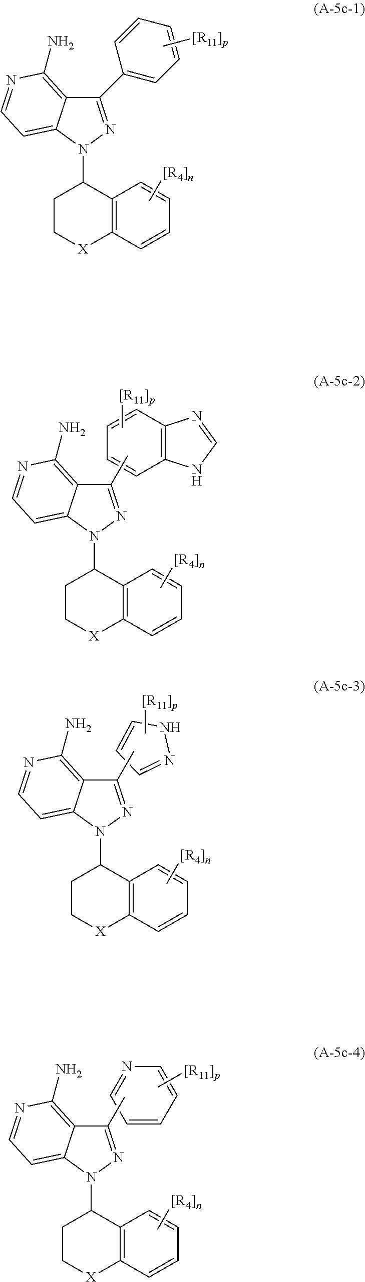

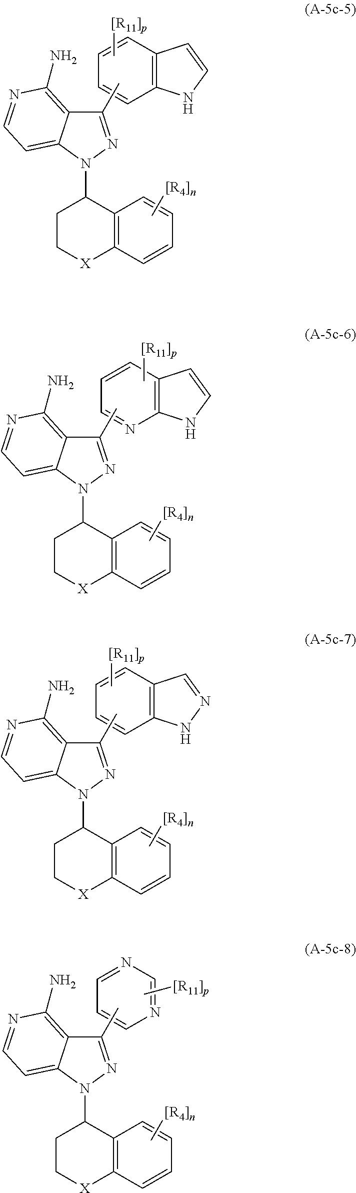

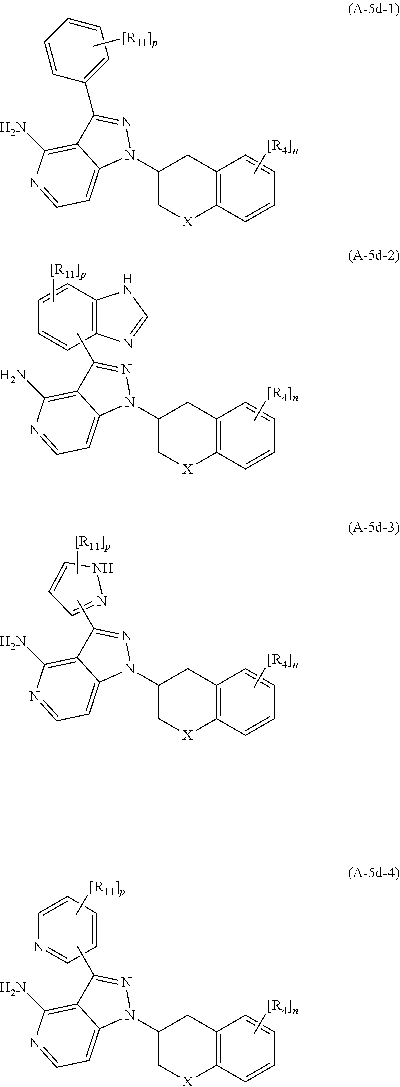

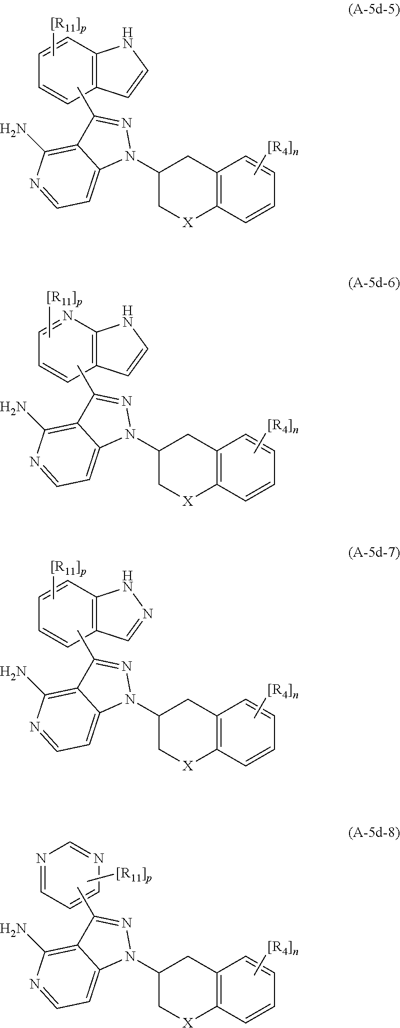

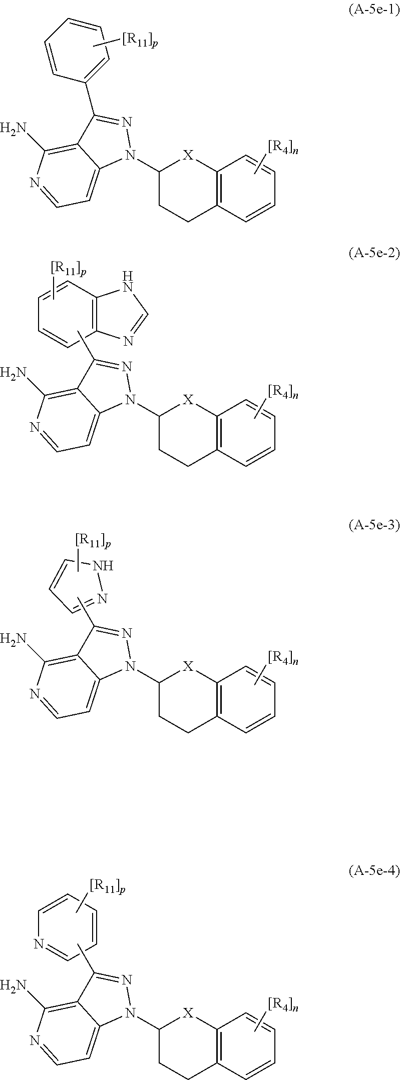

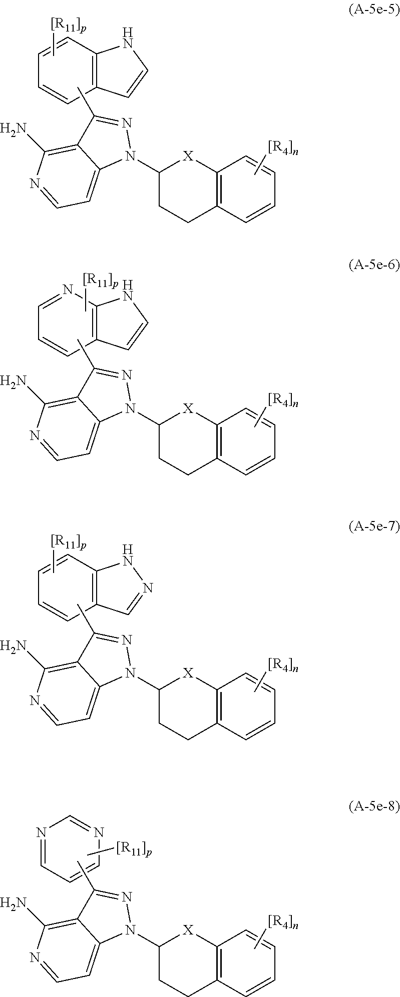

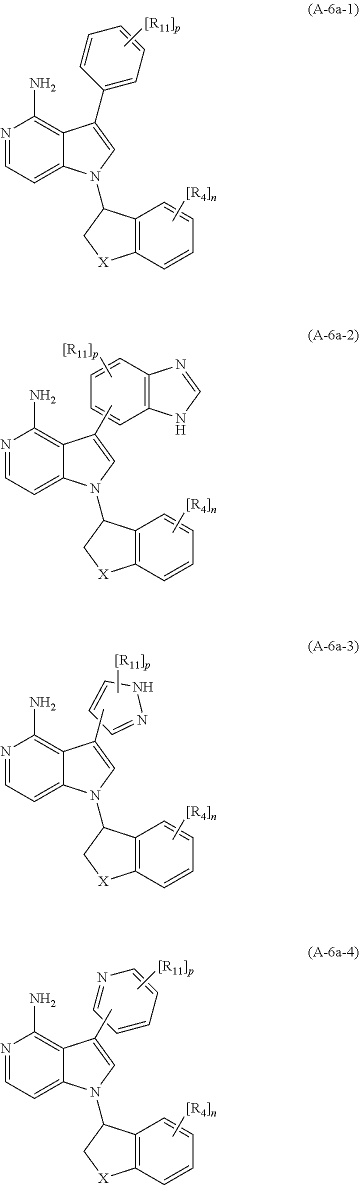

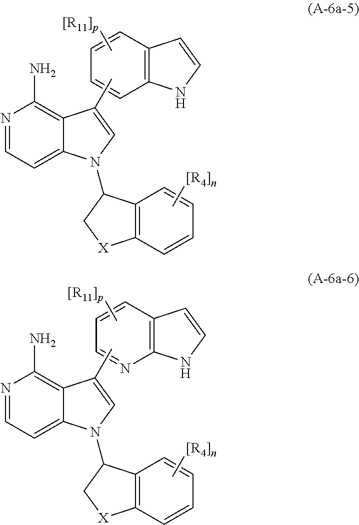

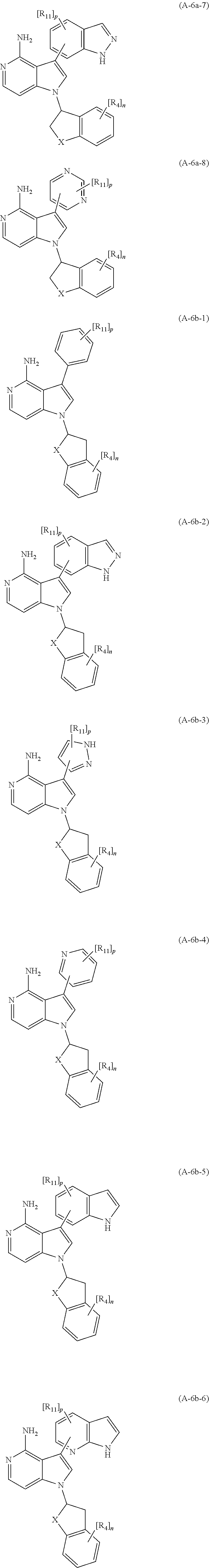

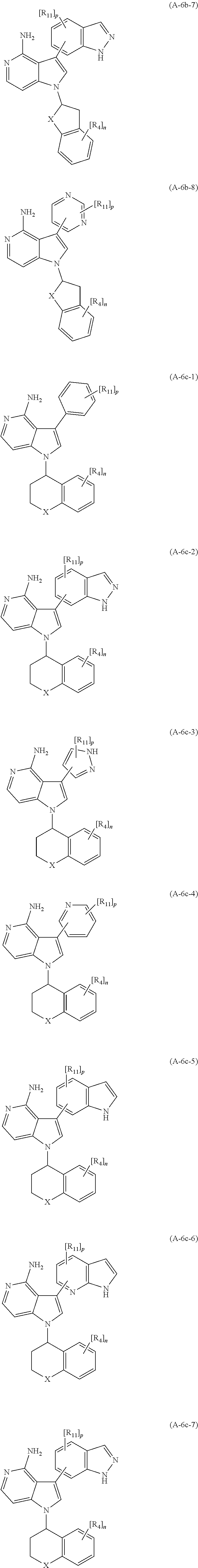

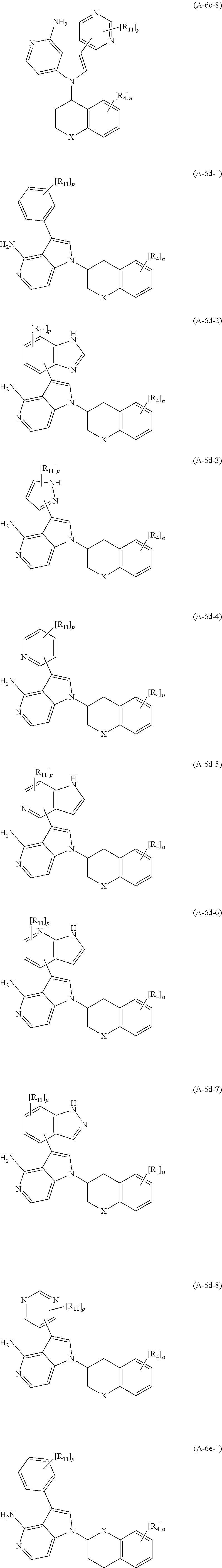

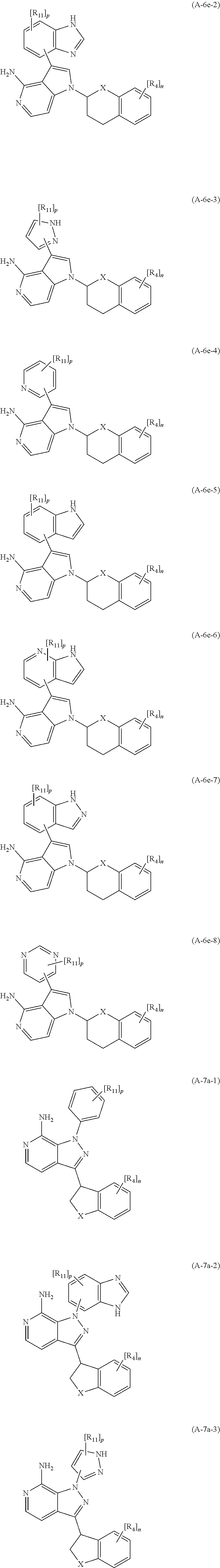

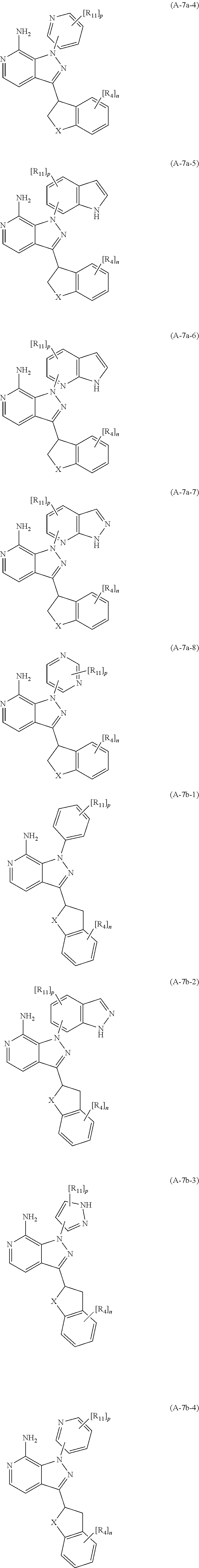

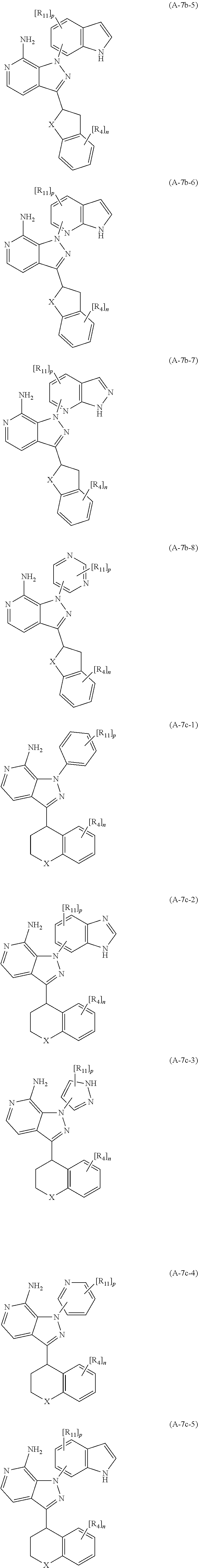

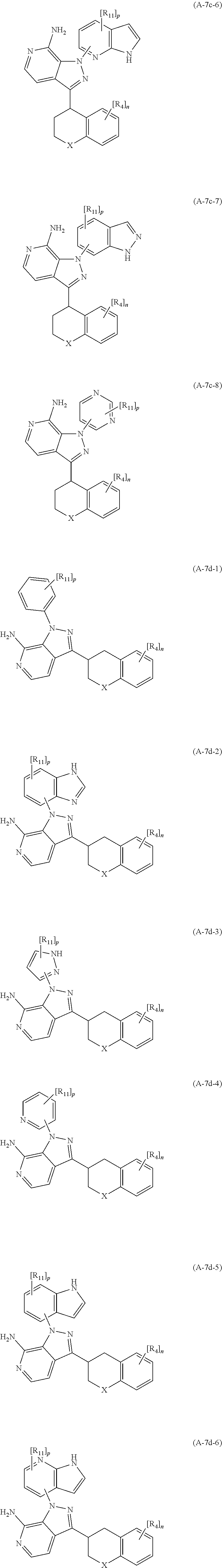

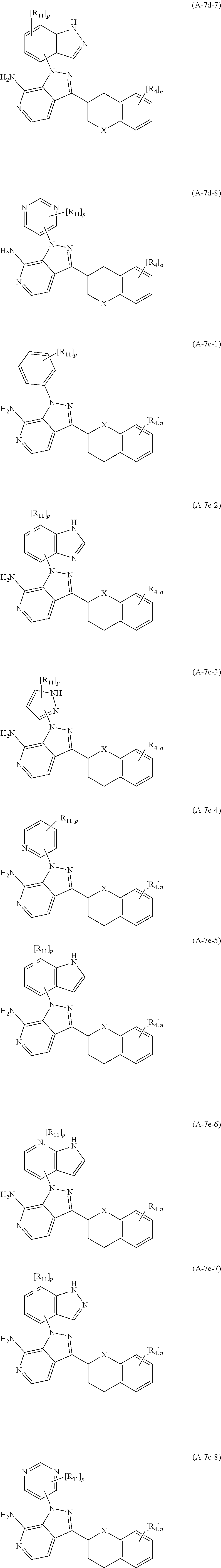

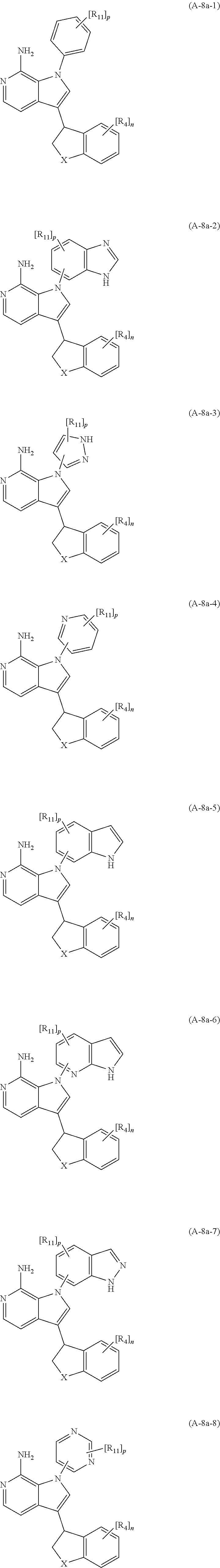

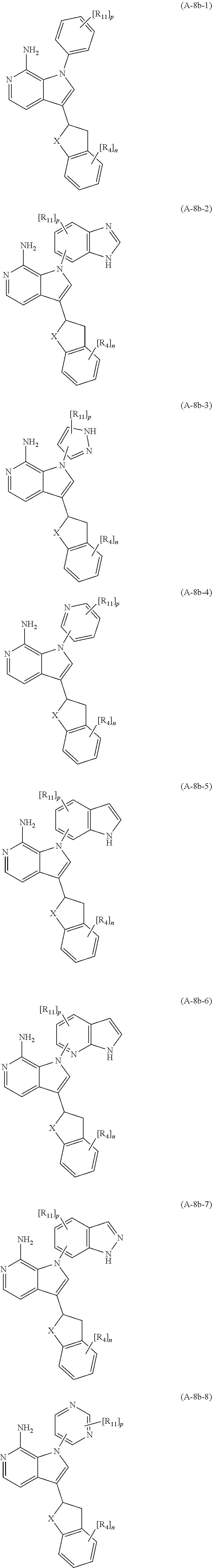

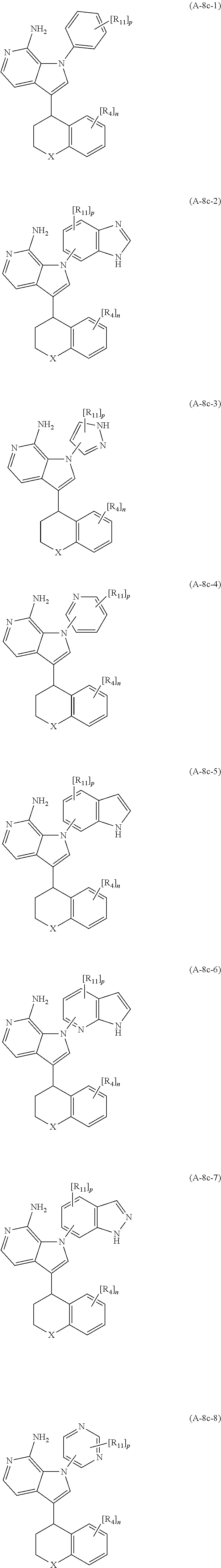

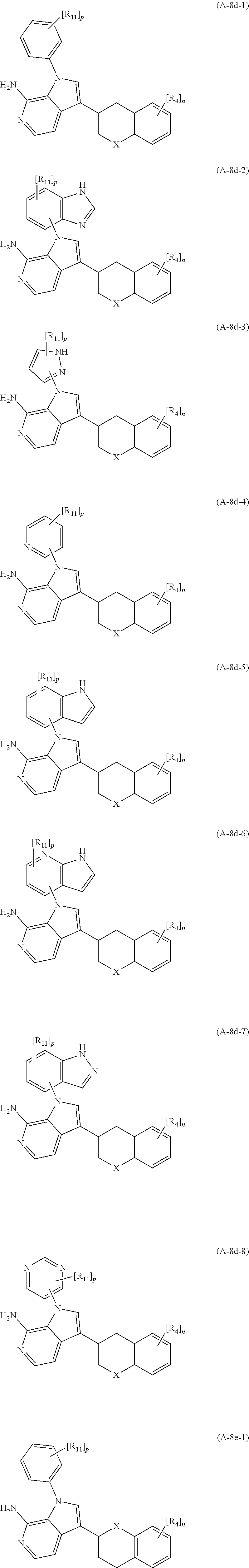

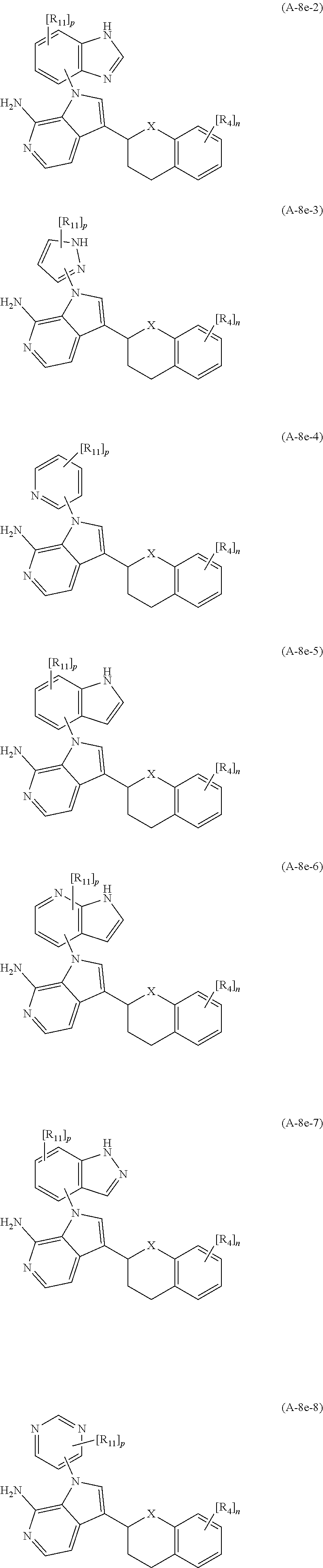

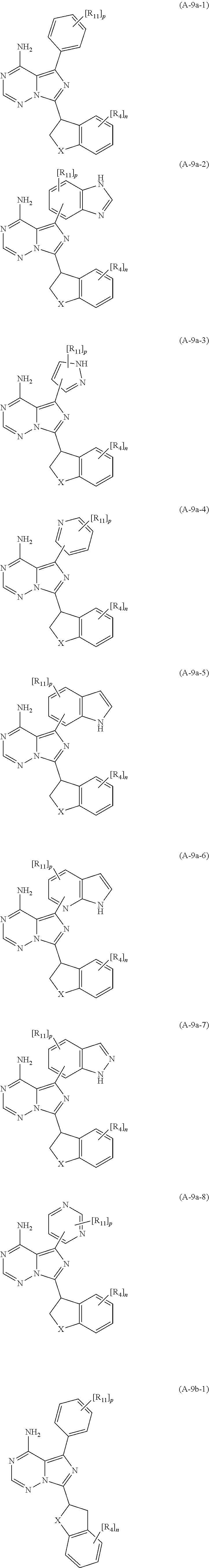

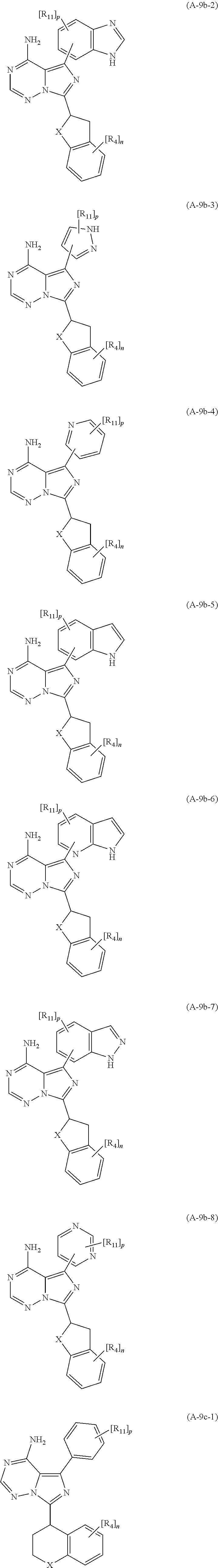

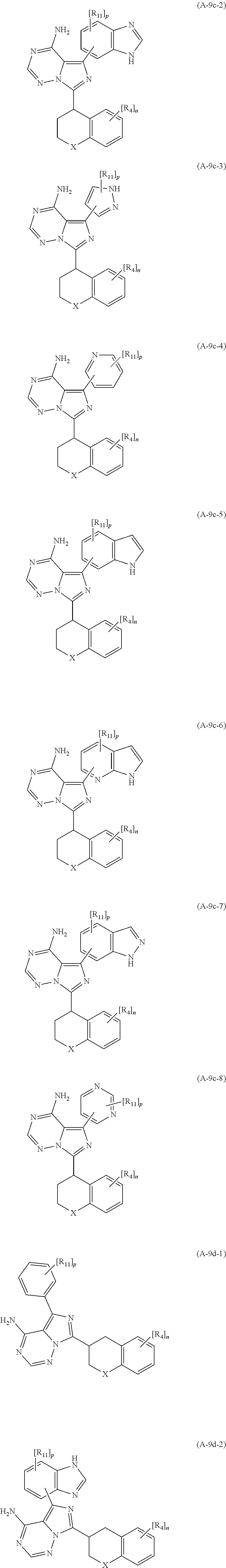

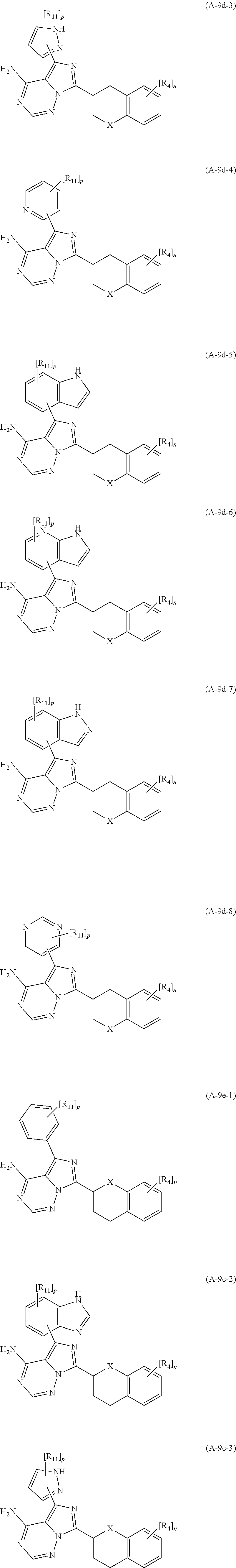

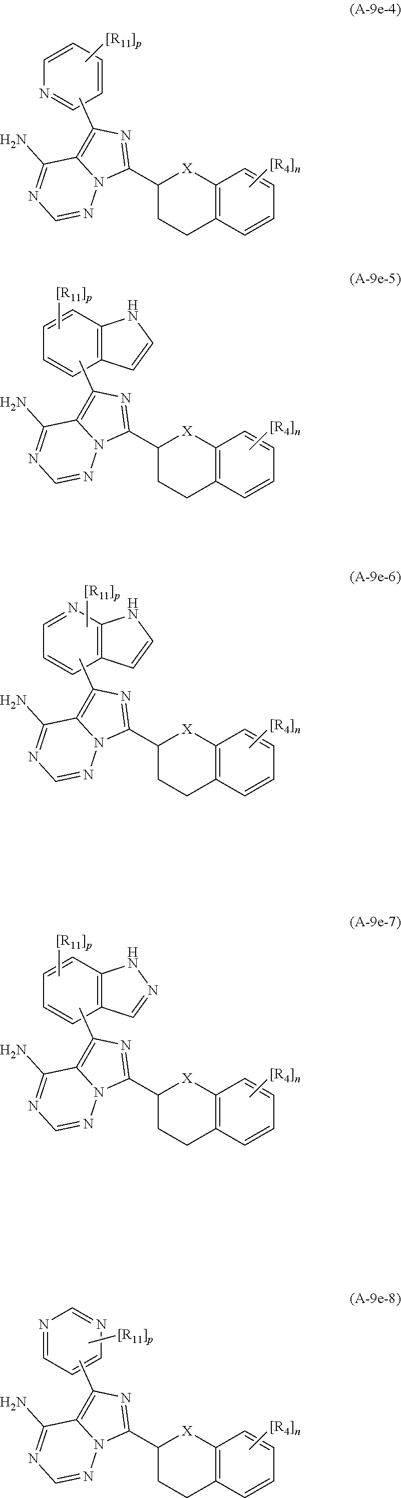

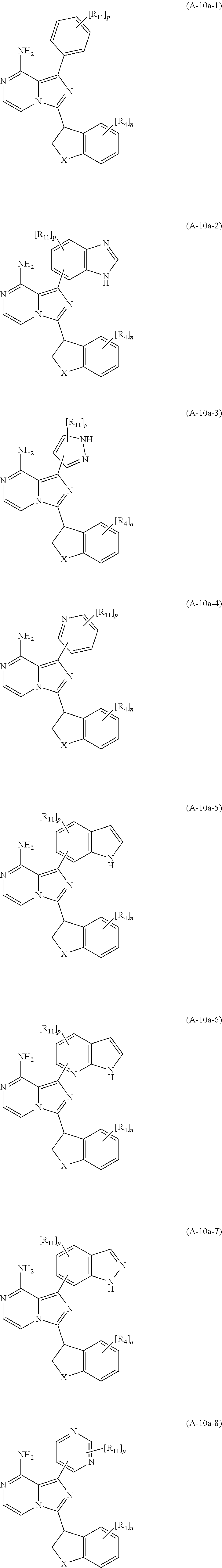

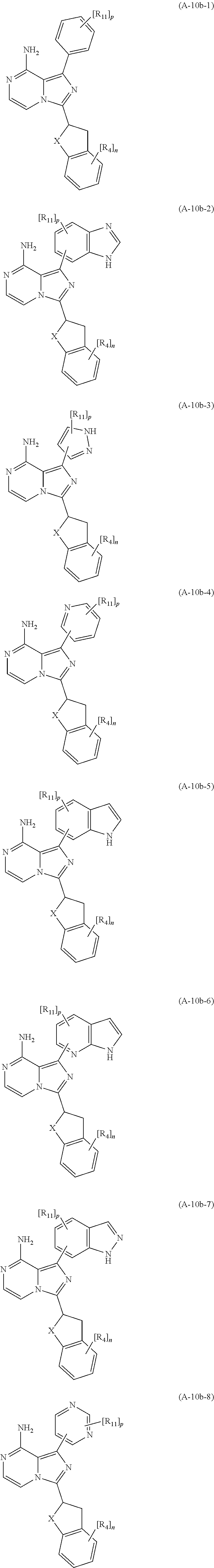

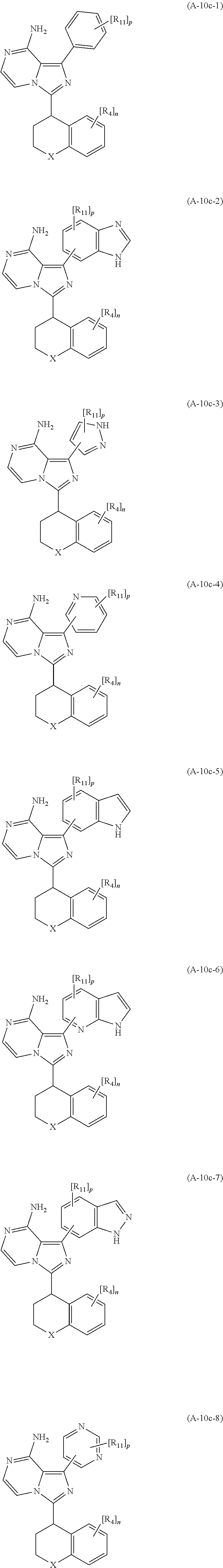

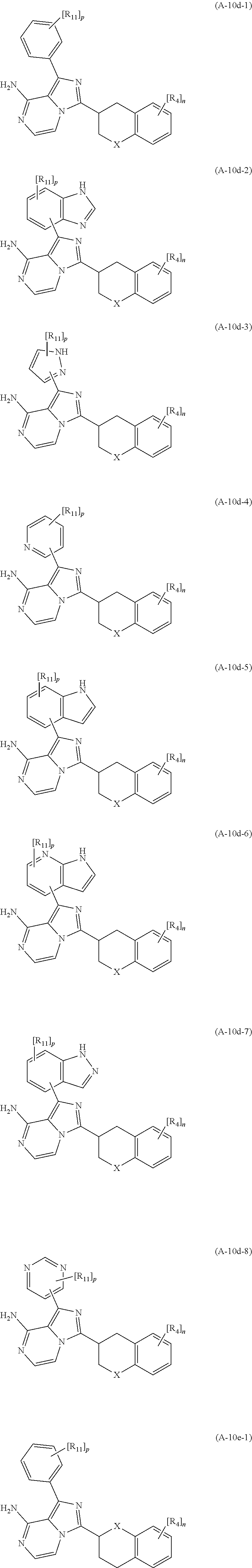

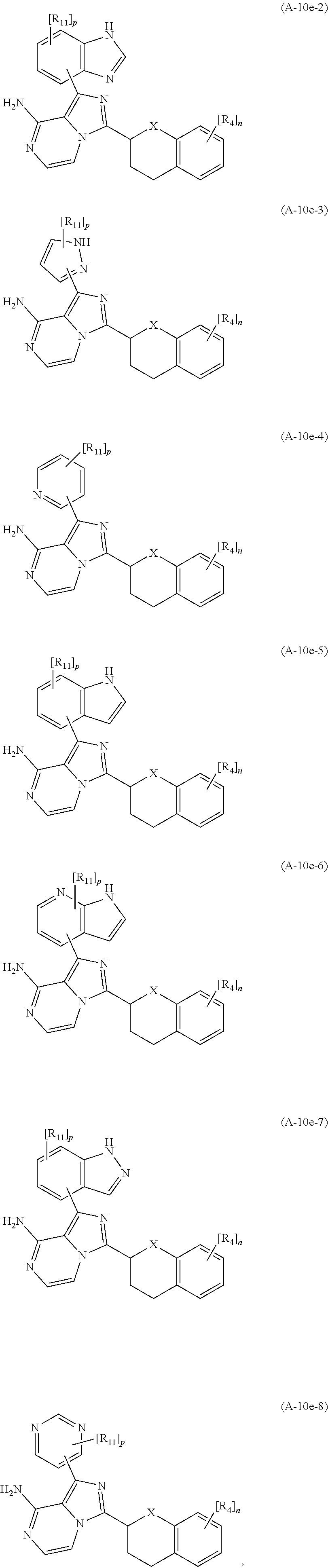

or a salt thereof; wherein R₄, X, n, R₅, R₆, R₇ and R₈, where present, are as described for formulae A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, and A-10e;

each R₁₁, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ or —NHR₅; and

p is 0 or an integer from 1 to 3.

In some embodiments of formulae A-1a-1 to A-10e-8, n is 0. In some embodiments, p is 0. In some embodiments, n is 0 and p is 0. In some embodiments, n is 0 and p is 1. In some embodiments, n is 0, and p is 2. In some embodiments, n is 0, and p is 3. In some embodiments, n is 1, and p is 0. In some embodiments, n is 1, and p is 1. In some embodiments, n is 1, and p is 2. In some embodiments, n is 1, and p is 3. In some embodiments, n is 2, and p is 0. In some embodiments, n is 2, and p is 1. In some embodiments, n is 2, and p is 2. In some embodiments, n is 2, and p is 3. In some embodiments, n is 3, and p is 0. In some embodiments, n is 3, and p is 1. In some embodiments, n is 3, and p is 2. In some embodiments, n is 3, and p is 3. In some embodiments, n is 4, and p is 0. In some embodiments, n is 4, and p is 1. In some embodiments, n is 4, and p is 2. In some embodiments, n is 4, and p is 3.

In some embodiments of formulae A-1a-1 to A-10e-8, X is CH₂. In some embodiments, X is NR₅, wherein R₅ is

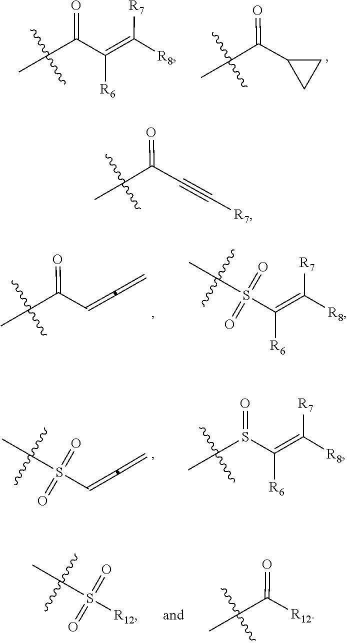

In some embodiments of formulae A-1a-1 to A-10e-8, R₁₁ is hydroxyl, halo, C₁-C₈ perhaloalkyl, substituted or unsubstituted C₁-C₈ alkyl, acyl, substituted or unsubstituted amino, acylamino, aminoacyl, —R₅ or —NHR₅. In some embodiments, R₁₁ is hydroxyl. In some embodiments, R₁₁ is fluoro, chloro or bromo. In some embodiments, R₁₁ is —R₅ or —NHR₅.

In some embodiments, the compound is of formulae A-1a-1 to A-1a-8, A-1b-1 to A-1b-8, A-1c-1 to A-1c-8, A-1d-1 to A-1d-8, or A-1e-1 to A-1e-8, wherein X is CH₂, n is 0, p is 0 or an integer from 1 to 3, and R₁₁ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, R₁₁ is selected from the group consisting of hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃.

In some embodiments, the compound is of formulae A-1a-1 to A-1a-8, A-1b-1 to A-1b-8, A-1c-1 to A-1c-8, A-1d-1 to A-1d-8, or A-1e-1 to A-1e-8, wherein X is CH₂, n is 0, p is 2, one R₁₁ is halo, and the remaining R₁₁ is hydroxy or alkoxy. In some embodiments, p is 2, and each R₁₁ is halo. In some embodiments, p is 2, and each R₁₁ is hydroxy. In some embodiments, p is 2, and each R₁₁ is alkoxy.

In some embodiments, the compound is of formulae A-1a-1 to A-1a-8, A-1b-1 to A-1b-8, A-1c-1 to A-1c-8, A-1d-1 to A-1d-8, or A-1e-1 to A-1e-8, wherein X is CH₂, n is 0, p is 3, one R₁₁ is halo, and the remaining two R₁₁ is hydroxy or alkoxy. In some embodiments, p is 3, two R₁₁ are each halo, and the remaining R₁₁ is hydroxy or alkoxy. In some embodiments, p is 3, two R₁₁ are each halo, and the remaining R₁₁ is hydroxy. In some embodiments, p is 3, one R₁₁ is cyano, one R₁₁ is halo, and the remaining R₁₁ is hydroxy or alkoxy.

In some embodiments, the compound is of formulae A-1a-1 to A-1a-8, A-1b-1 to A-1b-8, A-1c-1 to A-1c-8, A-1d-1 to A-1d-8, or A-1e-1 to A-1e-8, wherein X is CH₂, n is 0, p is 2, and two vicinal R₁₁ are taken together with the carbon atoms to which they are attached to form a substituted or unsubstituted heterocycle.

In some embodiments, the compound is of formulae A-1a-1 to A-1a-8, A-1b-1 to A-1b-8, A-1c-1 to A-1c-8, A-1d-1 to A-1d-8, or A-1e-1 to A-1e-8, wherein X is CH₂, n is 0, p is 3, two vicinal R₁₁ are taken together with the carbon atoms to which they are attached to form a substituted or unsubstituted heterocycle, and the remaining R₁₁ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, the remaining R₁₁ is halo, hydroxy, cyano, or methoxy.

In some embodiments, the compound is of formulae A-1a-1, A-1b-1, A-1c-1, or A-1d-1, wherein X is CH₂, n is 0, p is 0 or an integer from 1 to 3, and R₁₁ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, R₁₁ is selected from the group consisting of hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃.

In some embodiments, the compound is of formulae A-1a-1, A-1b-1, A-1c-1, or A-1d-1, wherein X is CH₂, n is 0, p is 2, one R₁₁ is halo, and the remaining R₁₁ is hydroxy or alkoxy. In some embodiments, p is 2, and each R₁₁ is halo. In some embodiments, p is 2, and each R₁₁ is hydroxy. In some embodiments, p is 2, and each R₁₁ is alkoxy.

In some embodiments, the compound is of formulae A-1a-1, A-1b-1, A-1c-1, or A-1d-1, wherein X is CH₂, n is 0, p is 3, one R₁₁ is halo, and the remaining two R₁₁ are each hydroxy or alkoxy. In some embodiments, p is 3, two R₁₁ are each halo, and the remaining R₁₁ is hydroxy or alkoxy. In some embodiments, p is 3, two R₁₁ are each halo, and the remaining R₁₁ is hydroxy. In some embodiments, p is 3, one R₁₁ is cyano, one R₁₁ is halo, and the remaining R₁₁ is hydroxy or alkoxy.

In some embodiments, the compound is of formulae A-1a-1, A-1b-1, A-1c-1, or A-1d-1, wherein X is CH₂, n is 0, p is 2, and two vicinal R₁₁ are taken together with the carbon atoms to which they are attached to form a substituted or unsubstituted heterocycle.

In some embodiments, the compound is of formulae A-1a-1, A-1b-1, A-1c-1, or A-1d-1, wherein X is CH₂, n is 0, p is 3, two vicinal R₁₁ are taken together with the carbon atoms to which they are attached to form a substituted or unsubstituted heterocycle, and the remaining R₁₁ is selected from the group consisting of hydroxyl, cyano, halo, C₁-C₈ perhaloalkyl, substituted or unsubstituted C₁-C₈ alkyl, C₁-C₈ perhaloalkoxy, C₁-C₈ alkoxy, substituted or unsubstituted amino, acylamino, and aminoacyl. In some of these embodiments, the remaining R₁₁ is halo, hydroxy, cyano, or methoxy.

In some embodiments, the compound of formula (I) has the structure (B-1), (B-2), (B-3), or (B-4):

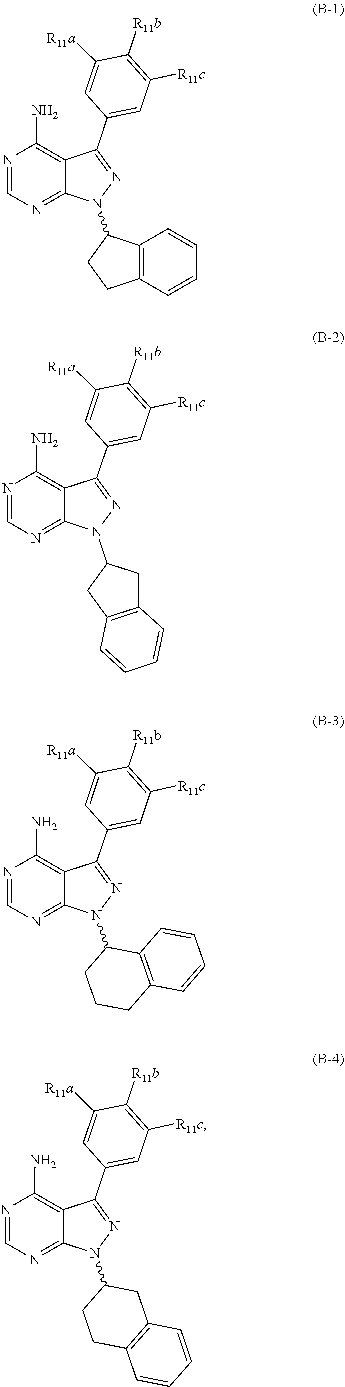

wherein each of R₁₁a, R₁₁b, and R₁₁c is independently selected from the group consisting of H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ and —NHR₅. In some of these embodiments, each R₁₁a, R₁₁b, and R₁₁c is independently selected from the group consisting of H, hydroxyl, cyano, halo, —CF₃, methyl, methoxy, ethoxy, isopropoxy, phenoxy, benzyloxy, —OCF₃, —CH₂OH, —CH₂NH₂, —NH₂, —NHCH₃, —CONH₂, and —NH(C═O)CH₃. In some of these embodiments, each R₁₁a, R₁₁b, and R₁₁c is independently selected from the group consisting of H, —CF₃, hydroxyl, cyano, and halo. In some of these embodiments, each R₁₁a, R₁₁b, and R₁₁c is independently selected from the group consisting of —CF₃, hydroxyl, cyano, and halo. In some of these embodiments, each R₁₁a, R₁₁b, and R₁₁c is independently selected from the group consisting of hydroxyl, and halo.

Variations of formulae (I), (II) and (III), detailed throughout, where applicable, apply to formulae A-1, A-2, A-3, A-4, A-5, A-6, A-7, A-8, A-9, and A-10, the same as if each and every variation were specifically and individually listed for formulae A-1, A-2, A-3, A-4, A-5, A-6, A-7, A-8, A-9, and A-10.

Variations of formulae A-1, A-2, A-3, A-4, A-5, A-6, A-7, A-8, A-9, and A-10, detailed throughout, where applicable, apply to formulae A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, or A-10e, the same as if each and every variation were specifically and individually listed for formulae A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, and A-10e.

Variations of formulae A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, and A-10e detailed throughout, where applicable, apply to formulae A-1a-1, A-1a-2, A-1a-3, A-1a-4, A-1a-5, A-1a-6, A-1a-7, A-1a-8, A-1b-1, A-1b-2, A-1b-3, A-1b-4, A-1b-5, A-1b-6, A-1b-7, A-1b-8, A-1c-1, A-1c-2, A-1c-3, A-1c-4, A-1c-5, A-1c-6, A-1c-7, A-1c-8, A-1d-1, A-1d-2, A-1d-3, A-1d-4, A-1d-5, A-1d-6, A-1d-7, A-1d-8, A-1e-1, A-1e-2, A-1e-3, A-1e-4, A-1e-5, A-1e-6, A-1e-7, A-1e-8, A-2a-1, A-2a-2, A-2a-3, A-2a-4, A-2a-5, A-2a-6, A-2a-7, A-2a-8, A-2b-1, A-2b-2, A-2b-3, A-2b-4, A-2b-5, A-2b-6, A-2b-7, A-2b-8, A-2c-1, A-2c-2, A-2c-3, A-2c-4, A-2c-5, A-2c-6, A-2c-7, A-2c-8, A-2d-1, A-2d-2, A-2d-3, A-2d-4, A-2d-5, A-2d-6, A-2d-7, A-2d-8, A-2e-1, A-2e-2, A-2e-3, A-2e-4, A-2e-5, A-2e-6, A-2e-7, A-2e-8, A-3a-1, A-3a-2, A-3a-3, A-3a-4, A-3a-5, A-3a-6, A-3a-7, A-3a-8, A-3b-1, A-3b-2, A-3b-3, A-3b-4, A-3b-5, A-3b-6, A-3b-7, A-3b-8, A-3c-1, A-3c-2, A-3c-3, A-3c-4, A-3c-5, A-3c-6, A-3c-7, A-3c-8, A-3d-1, A-3d-2, A-3d-3, A-3d-4, A-3d-5, A-3d-6, A-3d-7, A-3d-8, A-3e-1, A-3e-2, A-3e-3, A-3e-4, A-3e-5, A-3e-6, A-3e-7, A-3e-8, A-4a-1, A-4a-2, A-4a-3, A-4a-4, A-4a-5, A-4a-6, A-4a-7, A-4a-8, A-4b-1, A-4b-2, A-4b-3, A-4b-4, A-4b-5, A-4b-6, A-4b-7, A-4b-8, A-4c-1, A-4c-2, A-4c-3, A-4c-4, A-4c-5, A-4c-6, A-4c-7, A-4c-8, A-4d-1, A-4d-2, A-4d-3, A-4d-4, A-4d-5, A-4d-6, A-4d-7, A-4d-8, A-4e-1, A-4e-2, A-4e-3, A-4e-4, A-4e-5, A-4e-6, A-4e-7, A-4e-8, A-5a-1, A-5a-2, A-5a-3, A-5a-4, A-5a-5, A-5a-6, A-5a-7, A-5a-8, A-5b-1, A-5b-2, A-5b-3, A-5b-4, A-5b-5, A-5b-6, A-5b-7, A-5b-8, A-5c-1, A-5c-2, A-5c-3, A-5c-4, A-5c-5, A-5c-6, A-5c-7, A-5c-8, A-5d-1, A-5d-2, A-5d-3, A-5d-4, A-5d-5, A-5d-6, A-5d-7, A-5d-8, A-5e-1, A-5e-2, A-5e-3, A-5e-4, A-5e-5, A-5e-6, A-5e-7, A-5e-8, A-6a-1, A-6a-2, A-6a-3, A-6a-4, A-6a-5, A-6a-6, A-6a-7, A-6a-8, A-6b-1, A-6b-2, A-6b-3, A-6b-4, A-6b-5, A-6b-6, A-6b-7, A-6b-8, A-6c-1, A-6c-2, A-6c-3, A-6c-4, A-6c-5, A-6c-6, A-6c-7, A-6c-8, A-6d-1, A-6d-2, A-6d-3, A-6d-4, A-6d-5, A-6d-6, A-6d-7, A-6d-8, A-6e-1, A-6e-2, A-6e-3, A-6e-4, A-6e-5, A-6e-6, A-6e-7, A-6e-8, A-7a-1, A-7a-2, A-7a-3, A-7a-4, A-7a-5, A-7a-6, A-7a-7, A-7a-8, A-7b-1, A-7b-2, A-7b-3, A-7b-4, A-7b-5, A-7b-6, A-7b-7, A-7b-8, A-7c-1, A-7c-2, A-7c-3, A-7c-4, A-7c-5, A-7c-6, A-7c-7, A-7c-8, A-7d-1, A-7d-2, A-7d-3, A-7d-4, A-7d-5, A-7d-6, A-7d-7, A-7d-8, A-7e-1, A-7e-2, A-7e-3, A-7e-4, A-7e-5, A-7e-6, A-7e-7, A-7e-8, A-8a-1, A-8a-2, A-8a-3, A-8a-4, A-8a-5, A-8a-6, A-8a-7, A-8a-8, A-8b-1, A-8b-2, A-8b-3, A-8b-4, A-8b-5, A-8b-6, A-8b-7, A-8b-8, A-8c-1, A-8c-2, A-8c-3, A-8c-4, A-8c-5, A-8c-6, A-8c-7, A-8c-8, A-8d-1, A-8d-2, A-8d-3, A-8d-4, A-8d-5, A-8d-6, A-8d-7, A-8d-8, A-8e-1, A-8e-2, A-8e-3, A-8e-4, A-8e-5, A-8e-6, A-8e-7, A-8e-8, A-9a-1, A-9a-2, A-9a-3, A-9a-4, A-9a-5, A-9a-6, A-9a-7, A-9a-9, A-9b-1, A-9b-2, A-9b-3, A-9b-4, A-9b-5, A-9b-6, A-9b-7, A-9b-8, A-9c-1, A-9c-2, A-9c-3, A-9c-4, A-9c-5, A-9c-6, A-9c-7, A-9c-8, A-9d-1, A-9d-2, A-9d-3, A-9d-4, A-9d-5, A-9d-6, A-9d-7, A-9d-8, A-9e-1, A-9e-2, A-9e-3, A-9e-4, A-9e-5, A-9e-6, A-9e-7, A-9e-8, A-10a-1, A-10a-2, A-10a-3, A-10a-4, A-10a-5, A-10a-6, A-10a-7, A-10A-10, A-10b-1, A-10b-2, A-10b-3, A-10b-4, A-10b-5, A-10b-6, A-10b-7, A-10b-8, A-10c-1, A-10c-2, A-10c-3, A-10c-4, A-10c-5, A-10c-6, A-10c-7, A-10c-8, A-10d-1, A-10d-2, A-10d-3, A-10d-4, A-10d-5, A-10d-6, A-10d-7, A-10d-8, A-10e-1, A-10e-2, A-10e-3, A-10e-4, A-10e-5, A-10e-6, A-10e-7, and A-10e-8, the same as if each and every variation were specifically and individually listed for formulae A-1a-1, A-1a-2, A-1a-3, A-1a-4, A-1a-5, A-1a-6, A-1a-7, A-1a-8, A-1b-1, A-1b-2, A-1b-3, A-1b-4, A-1b-5, A-1b-6, A-1b-7, A-1b-8, A-1c-1, A-1c-2, A-1c-3, A-1c-4, A-1c-5, A-1c-6, A-1c-7, A-1c-8, A-1d-1, A-1d-2, A-1d-3, A-1d-4, A-1d-5, A-1d-6, A-1d-7, A-1d-8, A-1e-1, A-1e-2, A-1e-3, A-1e-4, A-1e-5, A-1e-6, A-1e-7, A-1e-8, A-2a-1, A-2a-2, A-2a-3, A-2a-4, A-2a-5, A-2a-6, A-2a-7, A-2a-8, A-2b-1, A-2b-2, A-2b-3, A-2b-4, A-2b-5, A-2b-6, A-2b-7, A-2b-8, A-2c-1, A-2c-2, A-2c-3, A-2c-4, A-2c-5, A-2c-6, A-2c-7, A-2c-8, A-2d-1, A-2d-2, A-2d-3, A-2d-4, A-2d-5, A-2d-6, A-2d-7, A-2d-8, A-2e-1, A-2e-2, A-2e-3, A-2e-4, A-2e-5, A-2e-6, A-2e-7, A-2e-8, A-3a-1, A-3a-2, A-3a-3, A-3a-4, A-3a-5, A-3a-6, A-3a-7, A-3a-8, A-3b-1, A-3b-2, A-3b-3, A-3b-4, A-3b-5, A-3b-6, A-3b-7, A-3b-8, A-3c-1, A-3c-2, A-3c-3, A-3c-4, A-3c-5, A-3c-6, A-3c-7, A-3c-8, A-3d-1, A-3d-2, A-3d-3, A-3d-4, A-3d-5, A-3d-6, A-3d-7, A-3d-8, A-3e-1, A-3e-2, A-3e-3, A-3e-4, A-3e-5, A-3e-6, A-3e-7, A-3e-8, A-4a-1, A-4a-2, A-4a-3, A-4a-4, A-4a-5, A-4a-6, A-4a-7, A-4a-8, A-4b-1, A-4b-2, A-4b-3, A-4b-4, A-4b-5, A-4b-6, A-4b-7, A-4b-8, A-4c-1, A-4c-2, A-4c-3, A-4c-4, A-4c-5, A-4c-6, A-4c-7, A-4c-8, A-4d-1, A-4d-2, A-4d-3, A-4d-4, A-4d-5, A-4d-6, A-4d-7, A-4d-8, A-4e-1, A-4e-2, A-4e-3, A-4e-4, A-4e-5, A-4e-6, A-4e-7, A-4e-8, A-5a-1, A-5a-2, A-5a-3, A-5a-4, A-5a-5, A-5a-6, A-5a-7, A-5a-8, A-5b-1, A-5b-2, A-5b-3, A-5b-4, A-5b-5, A-5b-6, A-5b-7, A-5b-8, A-5c-1, A-5c-2, A-5c-3, A-5c-4, A-5c-5, A-5c-6, A-5c-7, A-5c-8, A-5d-1, A-5d-2, A-5d-3, A-5d-4, A-5d-5, A-5d-6, A-5d-7, A-5d-8, A-5e-1, A-5e-2, A-5e-3, A-5e-4, A-5e-5, A-5e-6, A-5e-7, A-5e-8, A-6a-1, A-6a-2, A-6a-3, A-6a-4, A-6a-5, A-6a-6, A-6a-7, A-6a-8, A-6b-1, A-6b-2, A-6b-3, A-6b-4, A-6b-5, A-6b-6, A-6b-7, A-6b-8, A-6c-1, A-6c-2, A-6c-3, A-6c-4, A-6c-5, A-6c-6, A-6c-7, A-6c-8, A-6d-1, A-6d-2, A-6d-3, A-6d-4, A-6d-5, A-6d-6, A-6d-7, A-6d-8, A-6e-1, A-6e-2, A-6e-3, A-6e-4, A-6e-5, A-6e-6, A-6e-7, A-6e-8, A-7a-1, A-7a-2, A-7a-3, A-7a-4, A-7a-5, A-7a-6, A-7a-7, A-7a-8, A-7b-1, A-7b-2, A-7b-3, A-7b-4, A-7b-5, A-7b-6, A-7b-7, A-7b-8, A-7c-1, A-7c-2, A-7c-3, A-7c-4, A-7c-5, A-7c-6, A-7c-7, A-7c-8, A-7d-1, A-7d-2, A-7d-3, A-7d-4, A-7d-5, A-7d-6, A-7d-7, A-7d-8, A-7e-1, A-7e-2, A-7e-3, A-7e-4, A-7e-5, A-7e-6, A-7e-7, A-7e-8, A-8a-1, A-8a-2, A-8a-3, A-8a-4, A-8a-5, A-8a-6, A-8a-7, A-8a-8, A-8b-1, A-8b-2, A-8b-3, A-8b-4, A-8b-5, A-8b-6, A-8b-7, A-8b-8, A-8c-1, A-8c-2, A-8c-3, A-8c-4, A-8c-5, A-8c-6, A-8c-7, A-8c-8, A-8d-1, A-8d-2, A-8d-3, A-8d-4, A-8d-5, A-8d-6, A-8d-7, A-8d-8, A-8e-1, A-8e-2, A-8e-3, A-8e-4, A-8e-5, A-8e-6, A-8e-7, A-8e-8, A-9a-1, A-9a-2, A-9a-3, A-9a-4, A-9a-5, A-9a-6, A-9a-7, A-9a-9, A-9b-1, A-9b-2, A-9b-3, A-9b-4, A-9b-5, A-9b-6, A-9b-7, A-9b-8, A-9c-1, A-9c-2, A-9c-3, A-9c-4, A-9c-5, A-9c-6, A-9c-7, A-9c-8, A-9d-1, A-9d-2, A-9d-3, A-9d-4, A-9d-5, A-9d-6, A-9d-7, A-9d-8, A-9e-1, A-9e-2, A-9e-3, A-9e-4, A-9e-5, A-9e-6, A-9e-7, A-9e-8, A-10a-1, A-10a-2, A-10a-3, A-10a-4, A-10a-5, A-10a-6, A-10a-7, A-10A-10, A-10b-1, A-10b-2, A-10b-3, A-10b-4, A-10b-5, A-10b-6, A-10b-7, A-10b-8, A-10c-1, A-10c-2, A-10c-3, A-10c-4, A-10c-5, A-10c-6, A-10c-7, A-10c-8, A-10d-1, A-10d-2, A-10d-3, A-10d-4, A-10d-5, A-10d-6, A-10d-7, A-10d-8, A-10e-1, A-10e-2, A-10e-3, A-10e-4, A-10e-5, A-10e-6, A-10e-7, and A-10e-8.

In one embodiment, the invention relates to Compounds described in Table 1, and uses thereof.

In another embodiment, the invention relates to Compound Nos. 1, 1a, 1b, 2, 3, 4, 4a, 4b, 5, 6, 6a, 6b, 7, 7a, 7b, 8, 8a, 8b, 9, 9a, 9b, 10, 10a, 10b, 11, 11a, 11b, 12, 12a, 12b, 13, 13a, 13b, 14, 14a, 14b, 15, 15a, 15b, 16, 16a, 16b, 17, 17a, 17b, 18, 18a, 18b, 19, 19a, 19b, 20, 20a, 20b, 21, 21a, 21b, 22, 22a, 22b, 23, 23a, 23b, 24, 24a, 24b, 25, 25a, 25b, 26, 26a, 26b, 27, 27a, 27b, 28, 28a, 28b, 29, 30, 30a, 30b, 31, 31a, 31b, 32, 32a, 32b, 33, 33a, 33b, 34, 34a, 34b, 35, 36, 36a, 36b, 37, 37a, 37b, 38, 38a, 38b, 39, 40, 41, 41a, 41b, 42, 42a, 42b, 43, 44, 45, 46, 47, 48, 49, 50, 51, 51a, 51b, 52, 53, 54, 55, 56, 57, 57a, 57b, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 106a, 106b, 106c, 106d, 107, 108, 109, 110, 111, 112, 113, 114, 114a, 114b, 115, 116, 117, 118, 119, 119a, 119b, 120, 121, 122, 122a, 122b, 123, 123a, 123b, 124, 124a, 124b, 125, 126, 126a, 126b, 127, 127a, 127b, 128, 128a, 128b, 129, 130, 130a, 130b, 131, 131a, 131b, 132, 133, 134, 135, 136, 137, 138, 139, 139a, 139b, and 140, and uses thereof.

In another embodiment, the invention relates to Compounds described in Table 2, and uses thereof.

In another embodiment, the invention relates to Compound Nos. 2.1, 2.1a, 2.1b, 2.2, 2.2a, 2.2b, 2.3, 2.4, 2.4a, 2.4b, 2.5, 2.5a, 2.5b, 2.6, 2.6a, 2.6b, 2.7, 2.7a, 2.7b, 2.8, 2.8a, 2.8b, 2.9, 2.9a, 2.9b, 2.10, 2.10a, 2.10b, 2.11, 2.11a, 2.11b, 2.12, 2.12a, 2.12b, 2.13, 2.14, 2.15, 2.15a, 2.15b, 2.16, 2.16a, 2.16b, 2.17, 2.17a, 2.17b, 2.18, 2.18a, 2.18b, 2.19, 2.19a, 2.19b, 2.20, 2.20a, 2.20b, 2.21, 2.21a, 2.21b, 2.22, 2.22a, 2.22b, 2.23, 2.23a, 2.23b, 2.24, 2.24a, 2.24b, 2.25, 2.25a, 2.25b, 2.26, 2.26a, 2.26b, 2.27, 2.27a, 2.27b, 2.28, 2.28a, 2.28b, 2.29, 2.29a, 2.29b, 2.30, 2.30a, 2.30b, 2.31, 2.31a, 2.31b, 2.32, 2.32a, 2.32b, 2.33, 2.33a, 2.33b, 2.34, 2.34a, 2.34b, 2.35, 2.35a, 2.35b, 2.36, 2.36a, 2.36b, 2.37, 2.37a, 2.37b, 2.38, 2.38a, 2.38b, 2.39, 2.39a, 2.39b, 2.40, 2.40a, 2.40b, 2.41, 2.41a, 2.41b, 2.42, 2.42a, 2.42b, 2.43, 2.43a, 2.43b, 2.44, 2.44a, 2.44b, 2.45, 2.45a, 2.45b, 2.46, 2.46a, 2.46b, 2.47, 2.47a, 2.47b, 2.48, 2.48a, 2.48b, 2.49, 2.49a, 2.49b, 2.50, 2.50a, 2.50b, 2.51, 2.51a, 2.51b, 2.52, 2.52a, 2.52b, 2.53, 2.53a, 2.53b, 2.54, 2.54a, 2.54b, 2.55, 2.55a, 2.55b, 2.55c, 2.55d, 2.56, 2.56a, 2.56b, 2.57, 2.58, 2.59, 2.59a, 2.59b, 2.60, 2.60a, 2.60b, 2.61, 2.61a, 2.61b, 2.62, 2.62a, 2.62b, 2.63, 2.63a, 2.63b, 2.64, 2.64a, 2.64b, 2.65, 2.65a, 2.65b, 2.66, 2.66a, 2.66b, 2.67, 2.67a, 2.67b, 2.68, 2.68a, 2.68b, 2.69, 2.69a, 2.69b, 2.70, 2.70a, 2.70b, 2.71, 2.71a, 2.71b, 2.72, 2.72a, 2.72b, 2.73, 2.73a, 2.73b, 2.74, 2.74a, 2.74b, 2.75, 2.75a, 2.75b, 2.76, 2.76a, 2.76b, 2.77, 2.77a, 2.77b, 2.78, 2.78a, 2.78b, 2.79, 2.79a, 2.79b, 2.80, 2.80a, 2.80b, 2.81, 2.81a, 2.81b, 2.82, 2.82a, 2.82b, 2.83, 2.83a, 2.83b, 2.84, 2.84a, 2.84b, 2.85, 2.85a, 2.85b, 2.86, 2.86a, 2.86b, 2.87, 2.87a, 2.87b, 2.88, 2.88a, 2.88b, 2.89, 2.89a, 2.89b, 2.90, 2.90a, 2.90b, 2.91, 2.91a, 2.91b, 2.92, 2.92a, 2.92b, 2.93, 2.94, 2.95, 2.96, 2.97, 2.98, 2.99, 2.100, 2.101, 2.102, 2.103, 2.104, 2.104a, 2.104b, 2.105, 2.105a, 2.105b, 2.106, 2.106a, 2.106b, 2.107, 2.107a, 2.107b, 2.108, 2.109, 2.109a, 2.109b, 2.110, 2.110a, 2.110b, 2.111, 2.112, 2.112a, 2.112b, 2.113, 2.113a, 2.113b, 2.114, 2.114a, 2.114b, 2.115, 2.115a, 2.115b, 2.116, 2.116a, 2.116b, 2.117, 2.117a, 2.117b, 2.118, 2.118a, 2.118b, 2.119, 2.119a, 2.119b, 2.120, 2.120a, 2.120b, 2.121, 2.121a, 2.121b, 2.122, 2.122a, 2.122b, 2.123, 2.123a, 2.123b, 2.124, 2.124a, 2.124b, 2.125, 2.125a, 2.125b, 2.126, 2.126a, 2.126b, 2.127, 2.128, 2.129, 2.130, 2.131, 2.132, 2.133, 2.134, 2.135, 2.136, 2.137, 2.138, 2.139, 2.140, 2.141, 2.142, 2.143, 2.144, 2.144a, 2.144b, 2.145, 2.145a, 2.145b, 2.146, 2.146a, 2.146b, 2.147, 2.147a, 2.147b, 2.148, 2.148a, 2.148b, 2.149, 2.149a, 2.149b, 2.150, 2.150a, 2.150b, 2.151, 2.151a, 2.151b, 2.152, 2.152a, 2.152b, 2.153, 2.153a, 2.153b, 2.154, 2.154a, 2.154b, 2.155, 2.155a, 2.155b, 2.156, 2.156a, 2.156b, 2.157, 2.157a, 2.157b, 2.158, 2.158a, 2.158b, 2.159, 2.159a, 2.159b, 2.160, 2.160a, 2.160b, 2.161, 2.161a, 2.161b, 2.162, 2.162a, 2.162b, 2.163, 2.163a, 2.163b, 2.164, 2.164a, 2.164b, 2.165, 2.165a, 2.165b, 2.166, 2.166a, 2.166b, 2.167, 2.167a, 2.167b, 2.168, 2.168a, 2.168b, 2.169, 2.169a, 2.169b, 2.170, 2.170a, 2.170b, 2.171, 2.171a, 2.171b, 2.172, 2.172a, 2.172b, 2.173, 2.173a, 2.173b, 2.174, 2.174a, 2.174b, 2.175, 2.175a, 2.175b, 2.176, 2.176a, 2.176b, 2.177, 2.177a, 2.177b, 2.178, 2.178a, 2.178b, 2.179, 2.179a, 2.179b, 2.180, 2.180a, 2.180b, 2.181, 2.181a, 2.181b, 2.182, 2.182a, 2.182b, 2.183, 2.183a, 2.183b, 2.184, 2.184a, 2.184b, 2.185, 2.185a, 2.185b, 2.186, 2.186a, 2.186b, 2.187, 2.187a, 2.187b, 2.188, 2.188a, 2.188b, 2.189, 2.189a, 2.189b, 2.190, 2.190a, 2.190b, 2.191, 2.191a, 2.191b, 2.192, 2.192a, 2.192b, 2.193, 2.193a, 2.193b, 2.194, 2.194a, 2.194b, 2.195, 2.195a, 2.195b, 2.196, 2.196a, 2.196b, 2.197, 2.197a, 2.197b, 2.198, 2.198a, 2.198b, 2.199, 2.199a, 2.199b, 2.200, 2.200a, 2.200b, 2.201, 2.201a, 2.201b, 2.202, 2.202a, 2.202b, 2.203, 2.203a, 2.203b, 2.204, 2.204a, 2.204b, 2.205, 2.206, 2.207, 2.208, 2.209, 2.209a, 2.209b, 2.210, 2.210a, 2.210b, 2.211, 2.211a, 2.211b, 2.212, 2.212a, 2.212b, 2.213, 2.213a, 2.213b, 2.214, 2.214a, 2.214b, 2.215, 2.215a, 2.215b, 2.216, 2.216a, 2.216b, 2.217, 2.218, 2.219, 2.220, 2.221, 2.221a, 2.221b, 2.222, 2.222a, 2.222b, 2.223, 2.223a, 2.223b, 2.224, 2.224a, 2.224b, 2.225, 2.225a, 2.225b, 2.226, 2.226a, 2.226b, 2.227, 2.227a, 2.227b, 2.228, 2.228a, 2.228b, 2.229, 2.230, 2.231, 2.232, 2.233, 2.233a, 2.233b, 2.234, 2.234a, 2.234b, 2.235, 2.235a, 2.235b, 2.236, 2.236a, 2.236b, 2.237, 2.238, 2.239, 2.240, 2.241, 2.241a, 2.241b, 2.242, 2.242a, 2.242b, 2.243, 2.243a, 2.243b, 2.244, 2.244a, 2.244b, 2.245, 2.246, 2.247, 2.248, 2.249, 2.249a, 2.249b, 2.250, 2.250a, 2.250b, 2.251, 2.251a, 2.251b, 2.252, 2.252a, 2.252b, 2.253, 2.254, 2.255, 2.255a, 2.255b, 2.256, 2.256a, 2.256b, 2.257, 2.257a, 2.257b, 2.258, 2.258a, 2.258b, 2.259, 2.259a, 2.259b, 2.260, 2.260a, 2.260b, 2.261, 2.262, 2.262a, 2.262b, 2.263, 2.263a, 2.263b, 2.264, 2.264a, 2.264b, 2.265, 2.266, 2.266a, 2.266b, 2.267, 2.267a, 2.267b, 2.268, 2.268a, 2.268b, 2.269, 2.269a, 2.269b, 2.270, 2.270a, 2.270b, 2.271, 2.272, 2.273, 2.273a, 2.273b, 2.274, 2.274a, 2.274b, 2.275, 2.275a, 2.275b, 2.276, 2.276a, 2.276b, 2.277, 2.277a, 2.277b, 2.278, 2.278a, and 2.278b, and uses thereof.

Also provided are methods of using compounds described herein, including any formula detailed herein or specific compound detailed herein, in various therapeutic applications.

Representative compounds of the invention are shown in Tables 1 and 2.

TABLE 1
Representative Compounds of the Invention

| 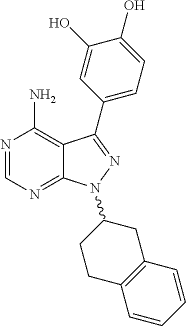 [SMILES: Nc1ncnc2c1c(-c1ccc(O)c(O)c1)nn2C1CCc2ccccc2C1] | 1 1a, 1b |
|---|---|
| 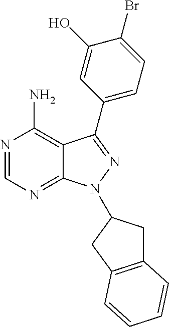 [SMILES: Nc1ncnc2c1c(-c1ccc(Br)c(O)c1)nn2C1Cc2ccccc2C1] | 2 |
| 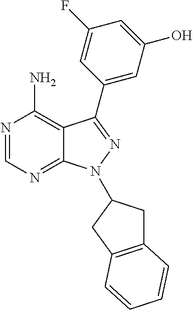 [SMILES: Nc1ncnc2c1c(-c1cc(O)cc(F)c1)nn2C1Cc2ccccc2C1] | 3 |
| 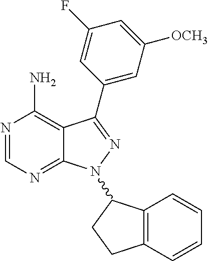 [SMILES: COc1cc(F)cc(-c2nn(C3CCc4ccccc43)c3ncnc(N)c23)c1] | 4 4a, 4b |
| 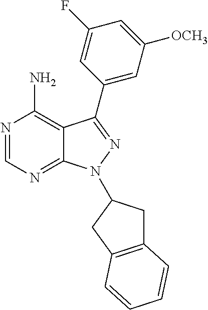 [SMILES: COc1cc(F)cc(-c2nn(C3Cc4ccccc4C3)c3ncnc(N)c23)c1] | 5 |
| 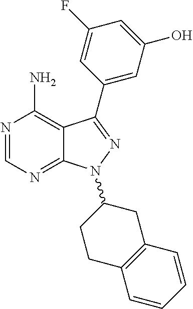 [SMILES: Nc1ncnc2c1c(-c1cc(O)cc(F)c1)nn2C1CCc2ccccc2C1] | 6 6a, 6b |
| 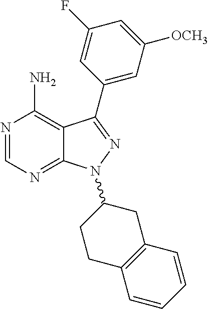 [SMILES: COc1cc(F)cc(-c2nn(C3CCc4ccccc4C3)c3ncnc(N)c23)c1] | 7 7a, 7b |
| 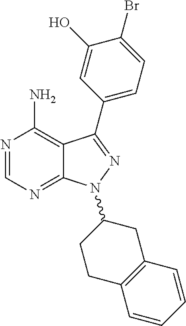 [SMILES: Nc1ncnc2c1c(-c1ccc(Br)c(O)c1)nn2C1CCc2ccccc2C1] | 8 8a, 8b |
| 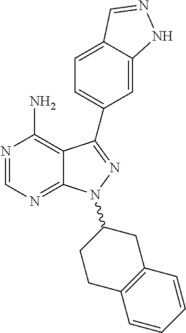 [SMILES: Nc1ncnc2c1c(-c1ccc3cn[nH]c3c1)nn2C1CCc2ccccc2C1] | 9 9a, 9b |
| 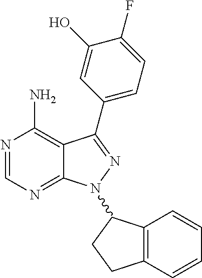 [SMILES: Nc1ncnc2c1c(-c1ccc(F)c(O)c1)nn2C1CCc2ccccc21] | 10 10a, 10b |
| 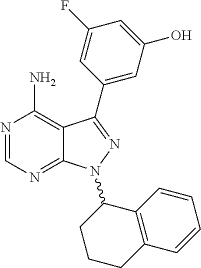 [SMILES: Nc1ncnc2c1c(-c1cc(O)cc(F)c1)nn2C1CCCc2ccccc21] | 11 11a, 11b |
| 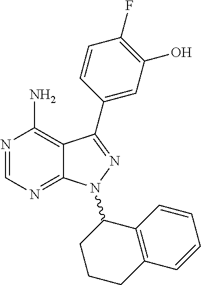 [SMILES: Nc1ncnc2c1c(-c1ccc(F)c(O)c1)nn2C1CCCc2ccccc21] | 12 12a, 12b |
| 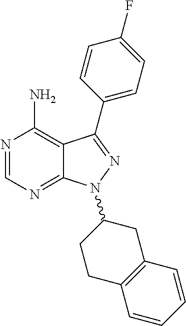 [SMILES: Nc1ncnc2c1c(-c1ccc(F)cc1)nn2C1CCc2ccccc2C1] | 13 13a, 13b |
| 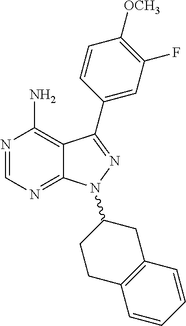 [SMILES: COc1ccc(-c2nn(C3CCc4ccccc4C3)c3ncnc(N)c23)cc1F] | 14 14a, 14b |
| 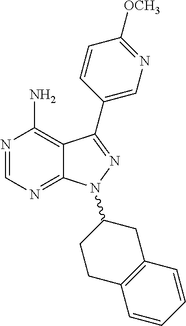 [SMILES: COc1ccc(-c2nn(C3CCc4ccccc4C3)c3ncnc(N)c23)cn1] | 15 15a, 15b |
| 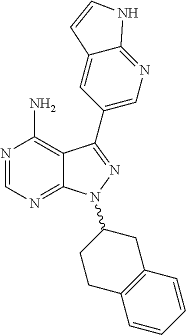 [SMILES: Nc1ncnc2c1c(-c1cnc3[nH]ccc3c1)nn2C1CCc2ccccc2C1] | 16 16a, 16b |
| 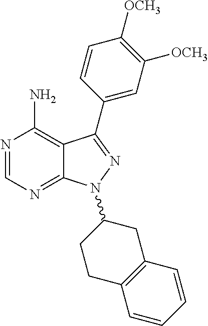 [SMILES: COc1ccc(-c2nn(C3CCc4ccccc4C3)c3ncnc(N)c23)cc1OC] | 17 17a, 17b |
| 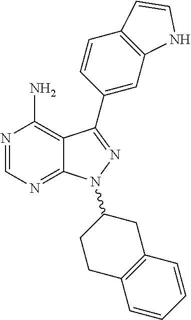 [SMILES: Nc1ncnc2c1c(-c1ccc3cc[nH]c3c1)nn2C1CCc2ccccc2C1] | 18 18a, 18b |
| 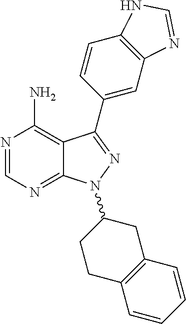 [SMILES: Nc1ncnc2c1c(-c1ccc3[nH]cnc3c1)nn2C1CCc2ccccc2C1] | 19 19a, 19b |
| 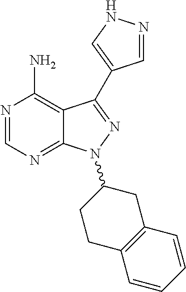 [SMILES: Nc1ncnc2c1c(-c1cn[nH]c1)nn2C1CCc2ccccc2C1] | 20 20a, 20b |
| 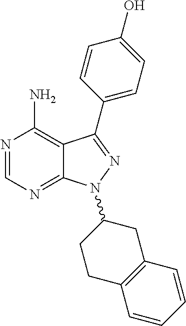 [SMILES: Nc1ncnc2c1c(-c1ccc(O)cc1)nn2C1CCc2ccccc2C1] | 21 21a, 21b |
| 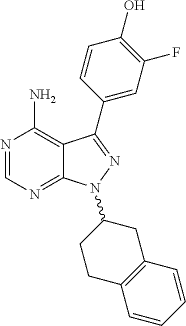 [SMILES: Nc1ncnc2c1c(-c1ccc(O)c(F)c1)nn2C1CCc2ccccc2C1] | 22 22a, 22b |
| 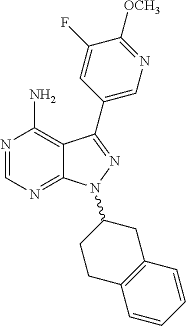 [SMILES: COc1ncc(-c2nn(C3CCc4ccccc4C3)c3ncnc(N)c23)cc1F] | 23 23a, 23b |
| 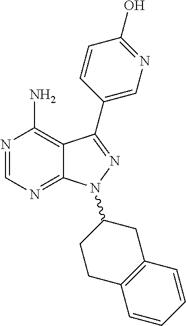 [SMILES: Nc1ncnc2c1c(-c1ccc(O)nc1)nn2C1CCc2ccccc2C1] | 24 24a, 24b |
| 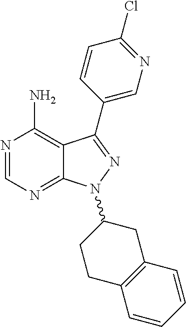 [SMILES: Nc1ncnc2c1c(-c1ccc(Cl)nc1)nn2C1CCc2ccccc2C1] | 25 25a, 25b |
| 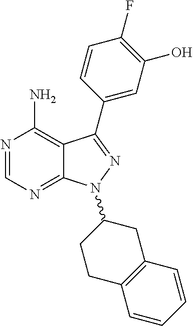 [SMILES: Nc1ncnc2c1c(-c1ccc(F)c(O)c1)nn2C1CCc2ccccc2C1] | 26 26a, 26b |
| 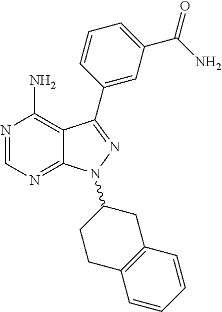 [SMILES: NC(=O)c1cccc(-c2nn(C3CCc4ccccc4C3)c3ncnc(N)c23)c1] | 27 27a, 27b |
| 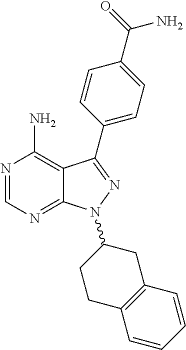 [SMILES: NC(=O)c1ccc(-c2nn(C3CCc4ccccc4C3)c3ncnc(N)c23)cc1] | 28 28a, 28b |
| 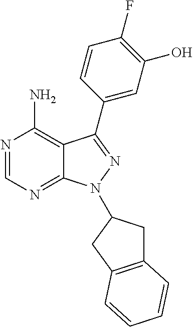 [SMILES: Nc1ncnc2c1c(-c1ccc(F)c(O)c1)nn2C1Cc2ccccc2C1] | 29 |
| 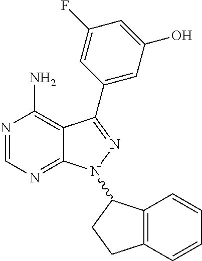 [SMILES: Nc1ncnc2c1c(-c1cc(O)cc(F)c1)nn2C1CCc2ccccc21] | 30 30a, 30b |
| 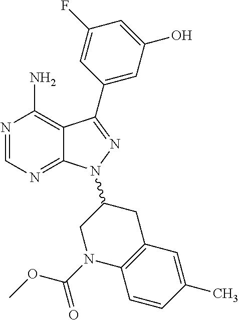 [SMILES: COC(=O)N1CC(n2nc(-c3cc(O)cc(F)c3)c3c(N)ncnc32)Cc2cc(C)ccc21] | 31 31a, 31b |
| 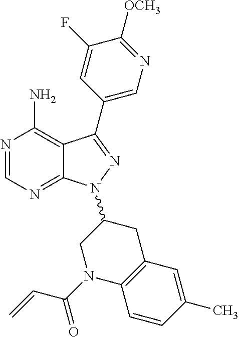 [SMILES: C=CC(=O)N1CC(n2nc(-c3cnc(OC)c(F)c3)c3c(N)ncnc32)Cc2cc(C)ccc21] | 32 32a, 32b |
| 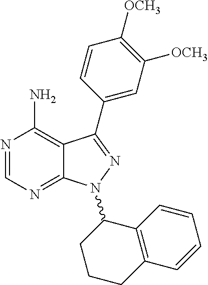 [SMILES: COc1ccc(-c2nn(C3CCCc4ccccc43)c3ncnc(N)c23)cc1OC] | 33 33a, 33b |
| 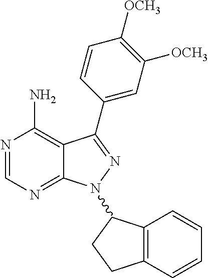 [SMILES: COc1ccc(-c2nn(C3CCc4ccccc43)c3ncnc(N)c23)cc1OC] | 34 34a, 34b |
| 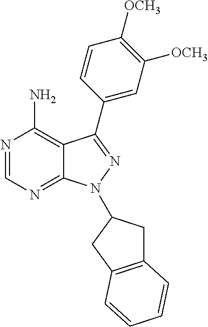 [SMILES: COc1ccc(-c2nn(C3Cc4ccccc4C3)c3ncnc(N)c23)cc1OC] | 35 |
| 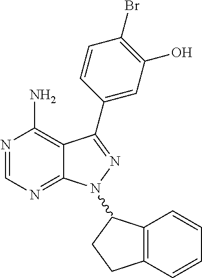 [SMILES: Nc1ncnc2c1c(-c1ccc(Br)c(O)c1)nn2C1CCc2ccccc21] | 36 36a, 36b |
| 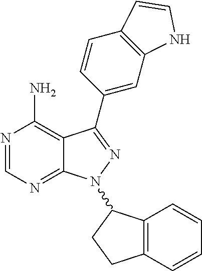 [SMILES: Nc1ncnc2c1c(-c1ccc3cc[nH]c3c1)nn2C1CCc2ccccc21] | 37 37a, 37b |
| 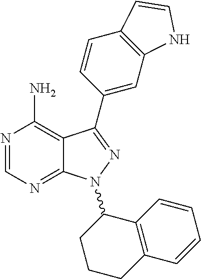 [SMILES: Nc1ncnc2c1c(-c1ccc3cc[nH]c3c1)nn2C1CCCc2ccccc21] | 38 38a, 38b |
| 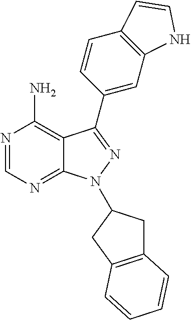 [SMILES: Nc1ncnc2c1c(-c1ccc3cc[nH]c3c1)nn2C1Cc2ccccc2C1] | 39 |
| 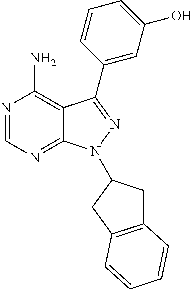 [SMILES: Nc1ncnc2c1c(-c1cccc(O)c1)nn2C1Cc2ccccc2C1] | 40 |
| 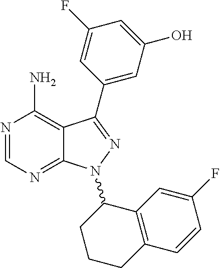 [SMILES: Nc1ncnc2c1c(-c1cc(O)cc(F)c1)nn2C1CCCc2ccc(F)cc21] | 41 41a, 41b |
| 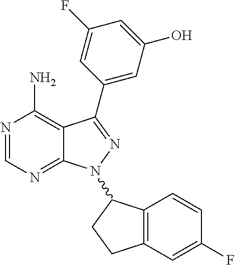 [SMILES: Nc1ncnc2c1c(-c1cc(O)cc(F)c1)nn2C1CCc2cc(F)ccc21] | 42 42a, 42b |
| 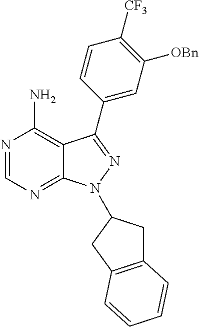 [SMILES: Nc1ncnc2c1c(-c1ccc(C(F)(F)F)c(OCc3ccccc3)c1)nn2C1Cc2ccccc2C1] | 43 |
| 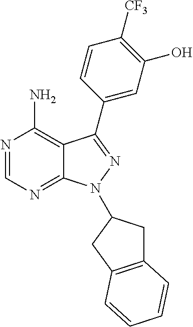 [SMILES: Nc1ncnc2c1c(-c1ccc(C(F)(F)F)c(O)c1)nn2C1Cc2ccccc2C1] | 44 |
| 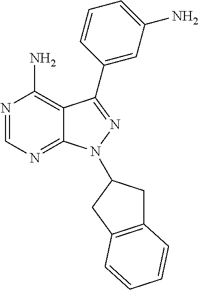 [SMILES: Nc1cccc(-c2nn(C3Cc4ccccc4C3)c3ncnc(N)c23)c1] | 45 |
| 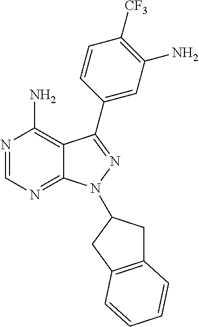 [SMILES: Nc1cc(-c2nn(C3Cc4ccccc4C3)c3ncnc(N)c23)ccc1C(F)(F)F] | 46 |
| 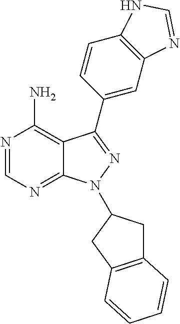 [SMILES: Nc1ncnc2c1c(-c1ccc3[nH]cnc3c1)nn2C1Cc2ccccc2C1] | 47 |
| 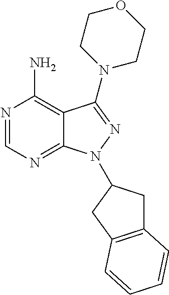 [SMILES: Nc1ncnc2c1c(N1CCOCC1)nn2C1Cc2ccccc2C1] | 48 |
| 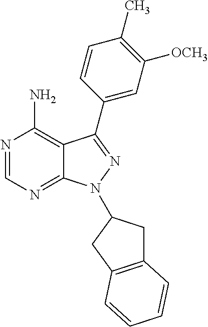 [SMILES: COc1cc(-c2nn(C3Cc4ccccc4C3)c3ncnc(N)c23)ccc1C] | 49 |
| 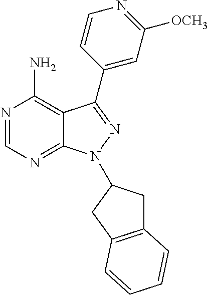 [SMILES: COc1cc(-c2nn(C3Cc4ccccc4C3)c3ncnc(N)c23)ccn1] | 50 |
| 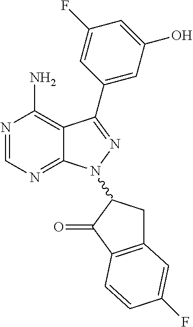 [SMILES: Nc1ncnc2c1c(-c1cc(O)cc(F)c1)nn2C1Cc2cc(F)ccc2C1=O] | 51 51a, 51b |
| 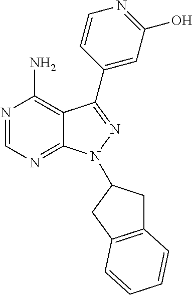 [SMILES: Nc1ncnc2c1c(-c1ccnc(O)c1)nn2C1Cc2ccccc2C1] | 52 |
| 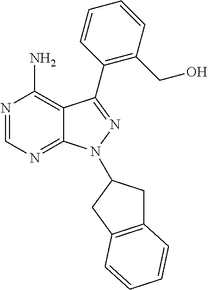 [SMILES: Nc1ncnc2c1c(-c1ccccc1CO)nn2C1Cc2ccccc2C1] | 53 |
| 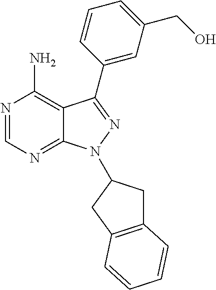 [SMILES: Nc1ncnc2c1c(-c1cccc(CO)c1)nn2C1Cc2ccccc2C1] | 54 |
| 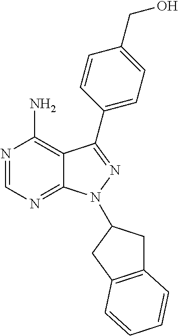 [SMILES: Nc1ncnc2c1c(-c1ccc(CO)cc1)nn2C1Cc2ccccc2C1] | 55 |
| 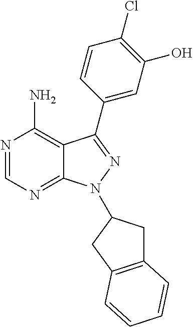 [SMILES: Nc1ncnc2c1c(-c1ccc(Cl)c(O)c1)nn2C1Cc2ccccc2C1] | 56 |
| 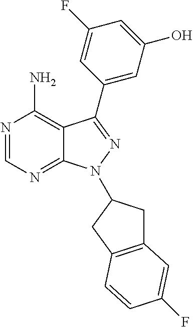 [SMILES: Nc1ncnc2c1c(-c1cc(O)cc(F)c1)nn2C1Cc2ccc(F)cc2C1] | 57 57a, 57b |
| 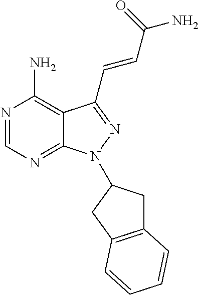 [SMILES: NC(=O)/C=C/c1nn(C2Cc3ccccc3C2)c2ncnc(N)c12] | 58 |
| 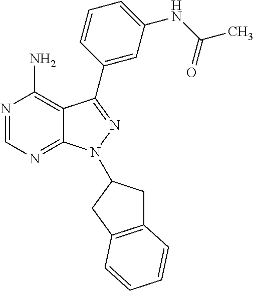 [SMILES: CC(=O)Nc1cccc(-c2nn(C3Cc4ccccc4C3)c3ncnc(N)c23)c1] | 59 |
| 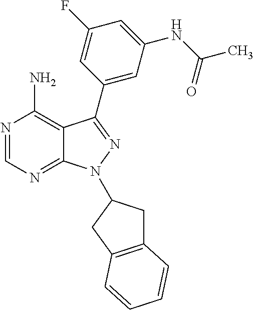 [SMILES: CC(=O)Nc1cc(F)cc(-c2nn(C3Cc4ccccc4C3)c3ncnc(N)c23)c1] | 60 |
| 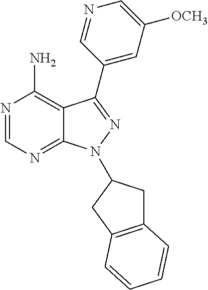 [SMILES: COc1cncc(-c2nn(C3Cc4ccccc4C3)c3ncnc(N)c23)c1] | 61 |
| 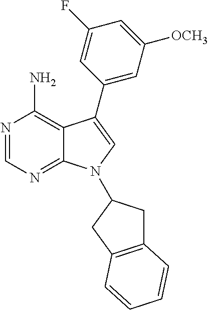 [SMILES: COc1cc(F)cc(-c2cn(C3Cc4ccccc4C3)c3ncnc(N)c23)c1] | 62 |
| 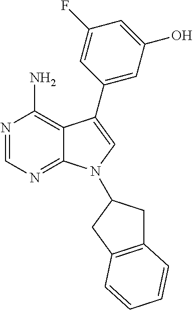 [SMILES: Nc1ncnc2c1c(-c1cc(O)cc(F)c1)cn2C1Cc2ccccc2C1] | 63 |
| 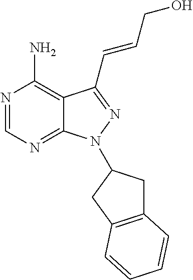 [SMILES: Nc1ncnc2c1c(/C=C/CO)nn2C1Cc2ccccc2C1] | 64 |
| 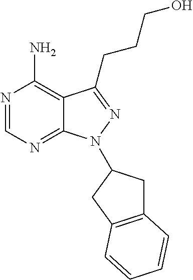 [SMILES: Nc1ncnc2c1c(CCCO)nn2C1Cc2ccccc2C1] | 65 |
| 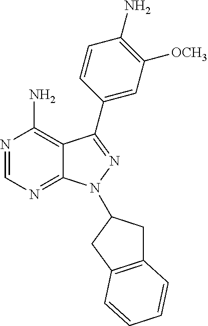 [SMILES: COc1cc(-c2nn(C3Cc4ccccc4C3)c3ncnc(N)c23)ccc1N] | 66 |
| 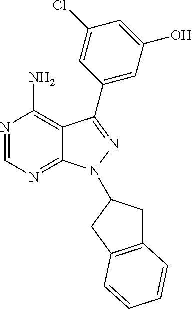 [SMILES: Nc1ncnc2c1c(-c1cc(O)cc(Cl)c1)nn2C1Cc2ccccc2C1] | 67 |
| 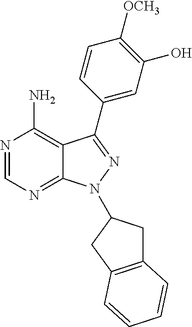 [SMILES: COc1ccc(-c2nn(C3Cc4ccccc4C3)c3ncnc(N)c23)cc1O] | 68 |
| 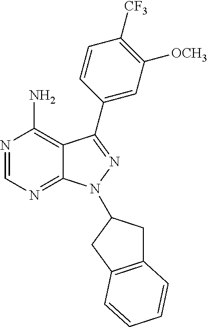 [SMILES: COc1cc(-c2nn(C3Cc4ccccc4C3)c3ncnc(N)c23)ccc1C(F)(F)F] | 69 |
| 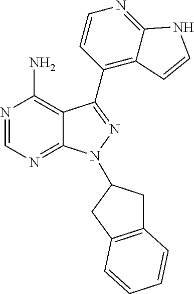 [SMILES: Nc1ncnc2c1c(-c1ccnc3[nH]ccc13)nn2C1Cc2ccccc2C1] | 70 |
| 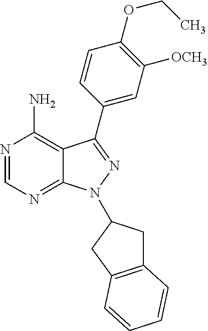 [SMILES: CCOc1ccc(-c2nn(C3Cc4ccccc4C3)c3ncnc(N)c23)cc1OC] | 71 |
| 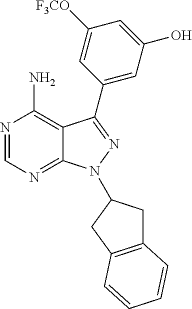 [SMILES: Nc1ncnc2c1c(-c1cc(O)cc(OC(F)(F)F)c1)nn2C1Cc2ccccc2C1] | 72 |
| 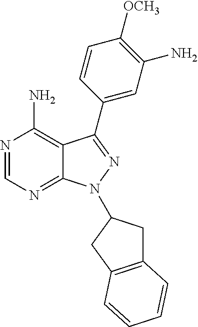 [SMILES: COc1ccc(-c2nn(C3Cc4ccccc4C3)c3ncnc(N)c23)cc1N] | 73 |
| 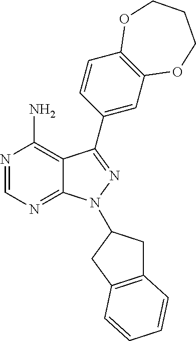 [SMILES: Nc1ncnc2c1c(-c1ccc3c(c1)OCCCO3)nn2C1Cc2ccccc2C1] | 74 |
| 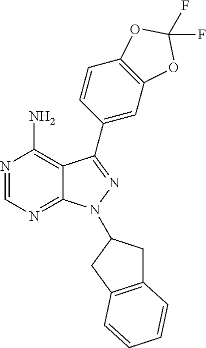 [SMILES: Nc1ncnc2c1c(-c1ccc3c(c1)OC(F)(F)O3)nn2C1Cc2ccccc2C1] | 75 |
| 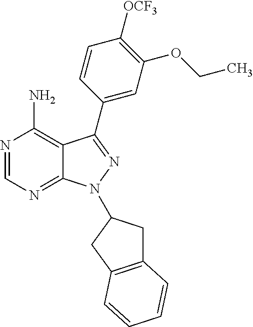 [SMILES: CCOc1cc(-c2nn(C3Cc4ccccc4C3)c3ncnc(N)c23)ccc1OC(F)(F)F] | 76 |
| 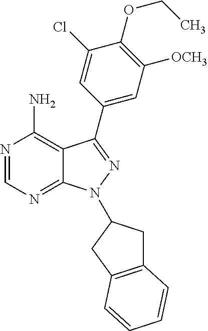 [SMILES: CCOc1c(Cl)cc(-c2nn(C3Cc4ccccc4C3)c3ncnc(N)c23)cc1OC] | 77 |
| 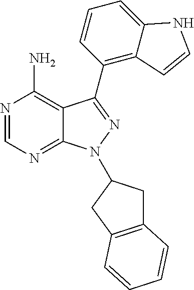 [SMILES: Nc1ncnc2c1c(-c1cccc3[nH]ccc13)nn2C1Cc2ccccc2C1] | 78 |
| 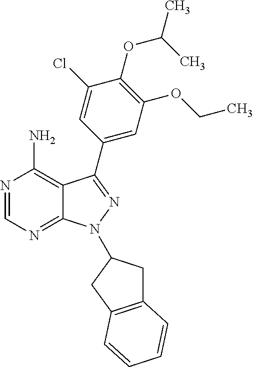 [SMILES: CCOc1cc(-c2nn(C3Cc4ccccc4C3)c3ncnc(N)c23)cc(Cl)c1OC(C)C] | 79 |
| 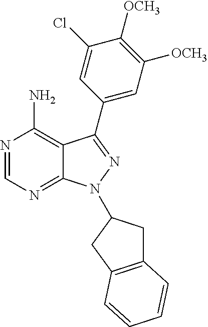 [SMILES: COc1cc(-c2nn(C3Cc4ccccc4C3)c3ncnc(N)c23)cc(Cl)c1OC] | 80 |
| 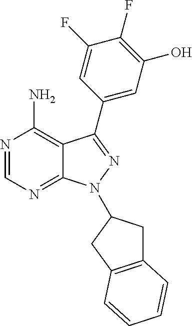 [SMILES: Nc1ncnc2c1c(-c1cc(O)c(F)c(F)c1)nn2C1Cc2ccccc2C1] | 81 |
| 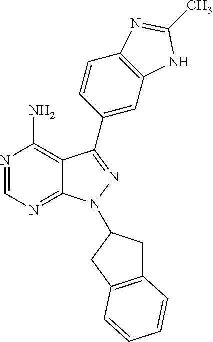 [SMILES: Cc1nc2ccc(-c3nn(C4Cc5ccccc5C4)c4ncnc(N)c34)cc2[nH]1] | 82 |
| 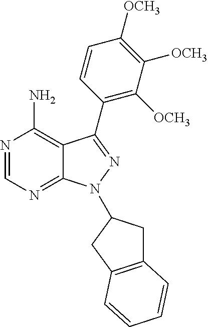 [SMILES: COc1ccc(-c2nn(C3Cc4ccccc4C3)c3ncnc(N)c23)c(OC)c1OC] | 83 |
| 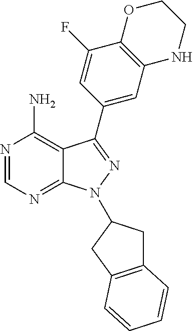 [SMILES: Nc1ncnc2c1c(-c1cc(F)c3c(c1)NCCO3)nn2C1Cc2ccccc2C1] | 84 |
| 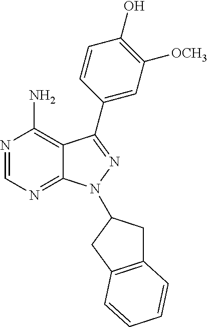 [SMILES: COc1cc(-c2nn(C3Cc4ccccc4C3)c3ncnc(N)c23)ccc1O] | 85 |
| 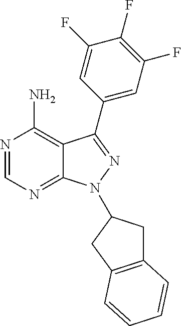 [SMILES: Nc1ncnc2c1c(-c1cc(F)c(F)c(F)c1)nn2C1Cc2ccccc2C1] | 86 |
| 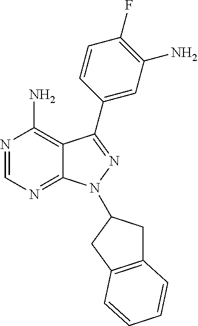 [SMILES: Nc1cc(-c2nn(C3Cc4ccccc4C3)c3ncnc(N)c23)ccc1F] | 87 |
| 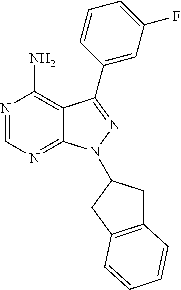 [SMILES: Nc1ncnc2c1c(-c1cccc(F)c1)nn2C1Cc2ccccc2C1] | 88 |
| 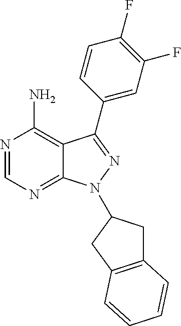 [SMILES: Nc1ncnc2c1c(-c1ccc(F)c(F)c1)nn2C1Cc2ccccc2C1] | 89 |
| 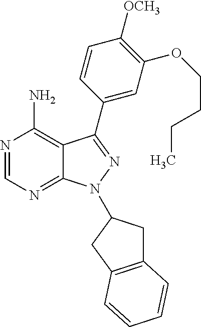 [SMILES: CCCCOc1cc(-c2nn(C3Cc4ccccc4C3)c3ncnc(N)c23)ccc1OC] | 90 |
| 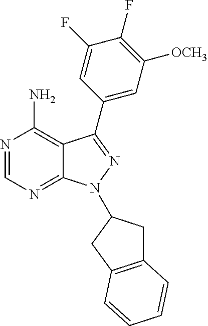 [SMILES: COc1cc(-c2nn(C3Cc4ccccc4C3)c3ncnc(N)c23)cc(F)c1F] | 91 |
| 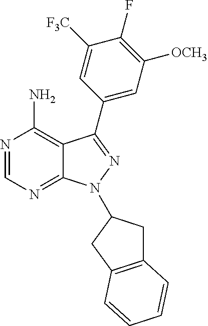 [SMILES: COc1cc(-c2nn(C3Cc4ccccc4C3)c3ncnc(N)c23)cc(C(F)(F)F)c1F] | 92 |
| 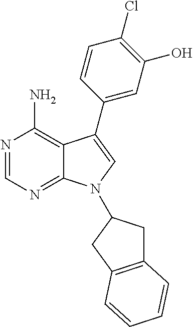 [SMILES: Nc1ncnc2c1c(-c1ccc(Cl)c(O)c1)cn2C1Cc2ccccc2C1] | 93 |
| 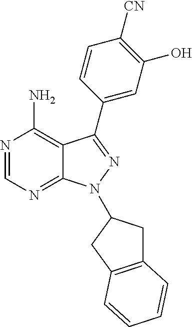 [SMILES: N#Cc1ccc(-c2nn(C3Cc4ccccc4C3)c3ncnc(N)c23)cc1O] | 94 |
| 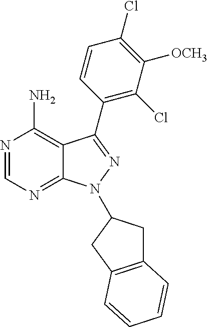 [SMILES: COc1c(Cl)ccc(-c2nn(C3Cc4ccccc4C3)c3ncnc(N)c23)c1Cl] | 95 |
| 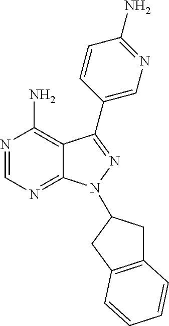 [SMILES: Nc1ccc(-c2nn(C3Cc4ccccc4C3)c3ncnc(N)c23)cn1] | 96 |
| 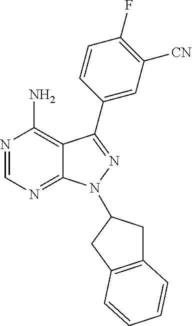 [SMILES: N#Cc1cc(-c2nn(C3Cc4ccccc4C3)c3ncnc(N)c23)ccc1F] | 97 |
| 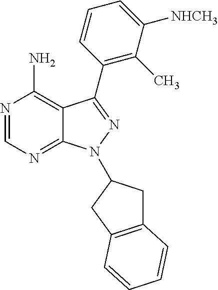 [SMILES: CNc1cccc(-c2nn(C3Cc4ccccc4C3)c3ncnc(N)c23)c1C] | 98 |
| 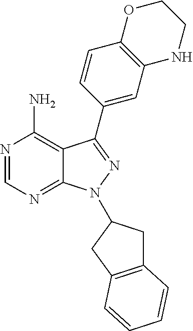 [SMILES: Nc1ncnc2c1c(-c1ccc3c(c1)NCCO3)nn2C1Cc2ccccc2C1] | 99 |
| 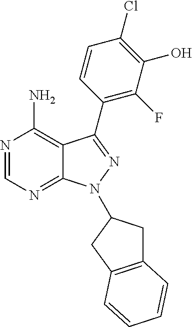 [SMILES: Nc1ncnc2c1c(-c1ccc(Cl)c(O)c1F)nn2C1Cc2ccccc2C1] | 100 |
| 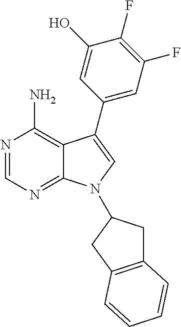 [SMILES: Nc1ncnc2c1c(-c1cc(O)c(F)c(F)c1)cn2C1Cc2ccccc2C1] | 101 |
| 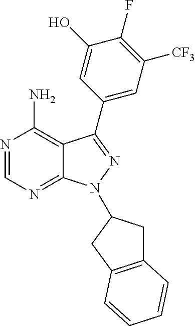 [SMILES: Nc1ncnc2c1c(-c1cc(O)c(F)c(C(F)(F)F)c1)nn2C1Cc2ccccc2C1] | 102 |
| 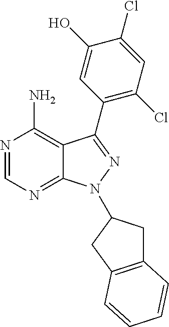 [SMILES: Nc1ncnc2c1c(-c1cc(O)c(Cl)cc1Cl)nn2C1Cc2ccccc2C1] | 103 |
| 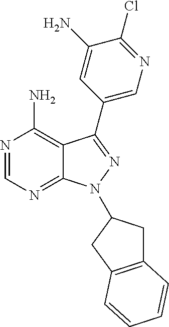 [SMILES: Nc1cc(-c2nn(C3Cc4ccccc4C3)c3ncnc(N)c23)cnc1Cl] | 104 |
| 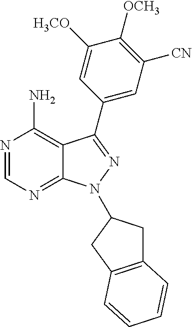 [SMILES: COc1cc(-c2nn(C3Cc4ccccc4C3)c3ncnc(N)c23)cc(C#N)c1OC] | 105 |
| 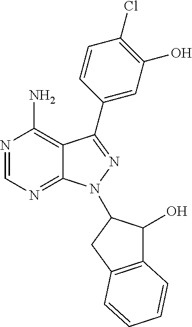 [SMILES: Nc1ncnc2c1c(-c1ccc(Cl)c(O)c1)nn2C1Cc2ccccc2C1O] | 106 106a, 106b, 106c, 106d |
| 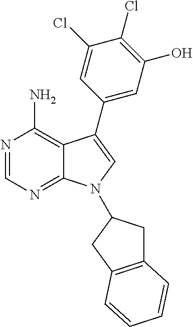 [SMILES: Nc1ncnc2c1c(-c1cc(O)c(Cl)c(Cl)c1)cn2C1Cc2ccccc2C1] | 107 |
| 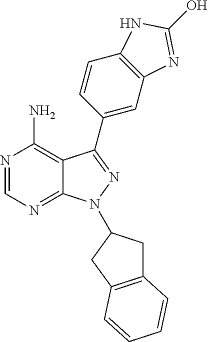 [SMILES: Nc1ncnc2c1c(-c1ccc3[nH]c(O)nc3c1)nn2C1Cc2ccccc2C1] | 108 |
| 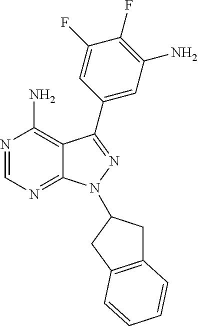 [SMILES: Nc1cc(-c2nn(C3Cc4ccccc4C3)c3ncnc(N)c23)cc(F)c1F] | 109 |
| 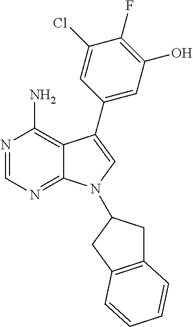 [SMILES: Nc1ncnc2c1c(-c1cc(O)c(F)c(Cl)c1)cn2C1Cc2ccccc2C1] | 110 |
| 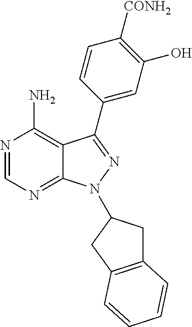 [SMILES: NC(=O)c1ccc(-c2nn(C3Cc4ccccc4C3)c3ncnc(N)c23)cc1O] | 111 |
| 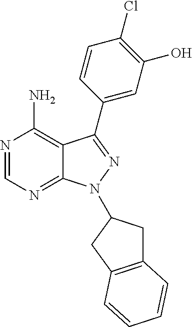 [SMILES: Nc1ncnc2c1c(-c1ccc(Cl)c(O)c1)nn2C1Cc2ccccc2C1] | 112 |
| 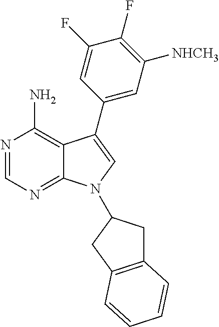 [SMILES: CNc1cc(-c2cn(C3Cc4ccccc4C3)c3ncnc(N)c23)cc(F)c1F] | 113 |
| 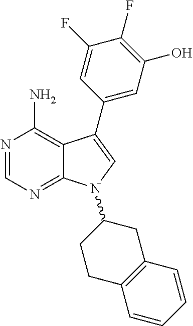 [SMILES: Nc1ncnc2c1c(-c1cc(O)c(F)c(F)c1)cn2C1CCc2ccccc2C1] | 114 114a, 114b |
| 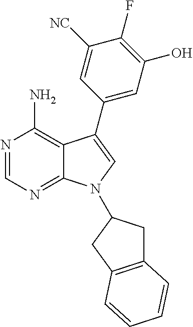 [SMILES: N#Cc1cc(-c2cn(C3Cc4ccccc4C3)c3ncnc(N)c23)cc(O)c1F] | 115 |
| 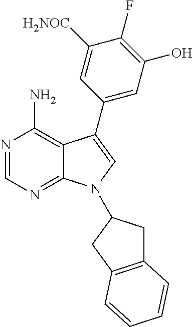 [SMILES: NC(=O)c1cc(-c2cn(C3Cc4ccccc4C3)c3ncnc(N)c23)cc(O)c1F] | 116 |
| 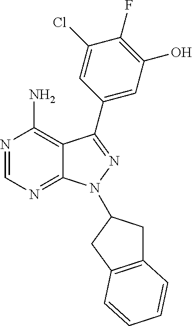 [SMILES: Nc1ncnc2c1c(-c1cc(O)c(F)c(Cl)c1)nn2C1Cc2ccccc2C1] | 117 |
| 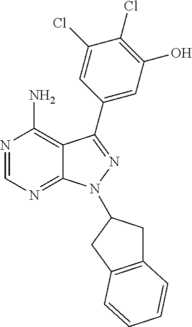 [SMILES: Nc1ncnc2c1c(-c1cc(O)c(Cl)c(Cl)c1)nn2C1Cc2ccccc2C1] | 118 |
| 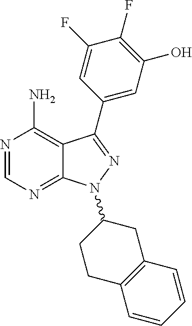 [SMILES: Nc1ncnc2c1c(-c1cc(O)c(F)c(F)c1)nn2C1CCc2ccccc2C1] | 119 119a, 119b |
| 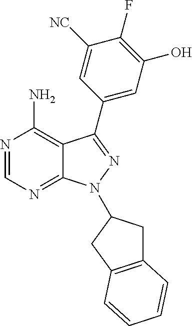 [SMILES: N#Cc1cc(-c2nn(C3Cc4ccccc4C3)c3ncnc(N)c23)cc(O)c1F] | 120 |
| 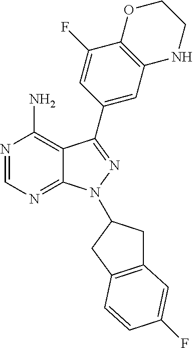 [SMILES: Nc1ncnc2c1c(-c1cc(F)c3c(c1)NCCO3)nn2C1Cc2ccc(F)cc2C1] | 121 |
| 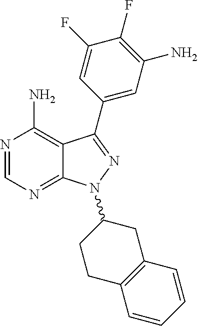 [SMILES: Nc1cc(-c2nn(C3CCc4ccccc4C3)c3ncnc(N)c23)cc(F)c1F] | 122 122a, 122b |
| 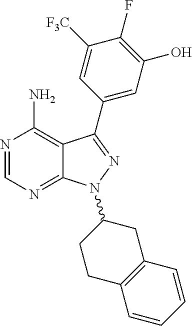 [SMILES: Nc1ncnc2c1c(-c1cc(O)c(F)c(C(F)(F)F)c1)nn2C1CCc2ccccc2C1] | 123 123a, 123b |
| 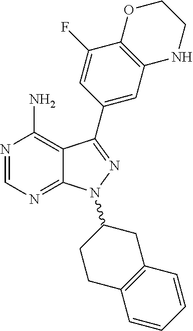 [SMILES: Nc1ncnc2c1c(-c1cc(F)c3c(c1)NCCO3)nn2C1CCc2ccccc2C1] | 124 124a, 124b |
| 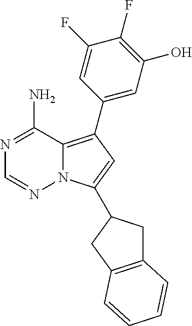 [SMILES: Nc1ncnn2c(C3Cc4ccccc4C3)cc(-c3cc(O)c(F)c(F)c3)c12] | 125 |
| 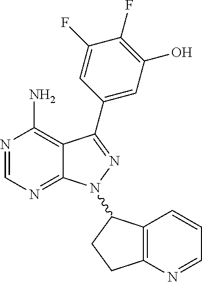 [SMILES: Nc1ncnc2c1c(-c1cc(O)c(F)c(F)c1)nn2C1CCc2ncccc21] | 126 126a, 126b |
| 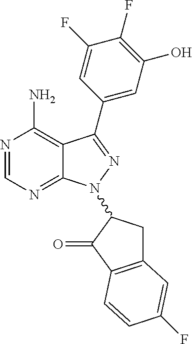 [SMILES: Nc1ncnc2c1c(-c1cc(O)c(F)c(F)c1)nn2C1Cc2cc(F)ccc2C1=O] | 127 127a, 127b |
| 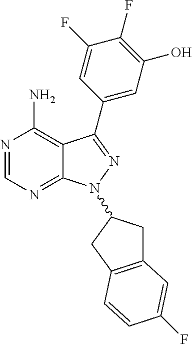 [SMILES: Nc1ncnc2c1c(-c1cc(O)c(F)c(F)c1)nn2C1Cc2ccc(F)cc2C1] | 128 128a, 128b |
| 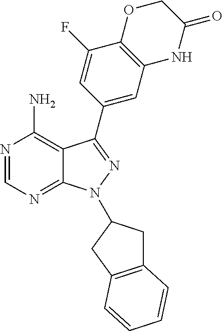 [SMILES: Nc1ncnc2c1c(-c1cc(F)c3c(c1)NC(=O)CO3)nn2C1Cc2ccccc2C1] | 129 |
| 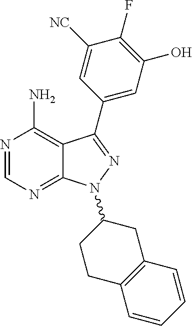 [SMILES: N#Cc1cc(-c2nn(C3CCc4ccccc4C3)c3ncnc(N)c23)cc(O)c1F] | 130 130a, 130b |
| 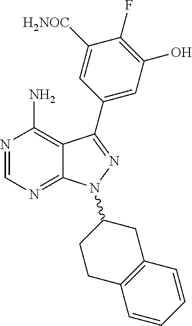 [SMILES: NC(=O)c1cc(-c2nn(C3CCc4ccccc4C3)c3ncnc(N)c23)cc(O)c1F] | 131 131a, 131b |
| 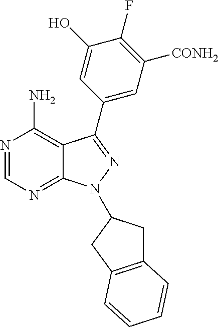 [SMILES: NC(=O)c1cc(-c2nn(C3Cc4ccccc4C3)c3ncnc(N)c23)cc(O)c1F] | 132 |
| 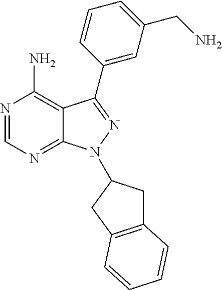 [SMILES: NCc1cccc(-c2nn(C3Cc4ccccc4C3)c3ncnc(N)c23)c1] | 133 |
| 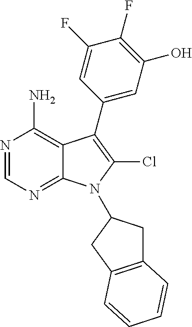 [SMILES: Nc1ncnc2c1c(-c1cc(O)c(F)c(F)c1)c(Cl)n2C1Cc2ccccc2C1] | 134 |
| 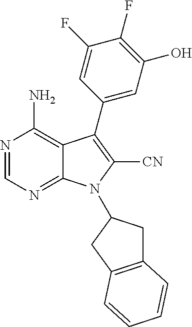 [SMILES: N#Cc1c(-c2cc(O)c(F)c(F)c2)c2c(N)ncnc2n1C1Cc2ccccc2C1] | 135 |
| 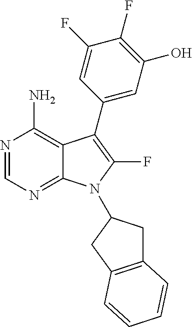 [SMILES: Nc1ncnc2c1c(-c1cc(O)c(F)c(F)c1)c(F)n2C1Cc2ccccc2C1] | 136 |
| 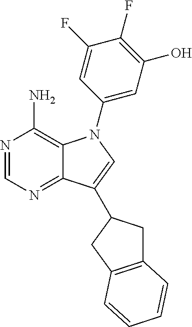 [SMILES: Nc1ncnc2c(C3Cc4ccccc4C3)cn(-c3cc(O)c(F)c(F)c3)c12] | 137 |
| 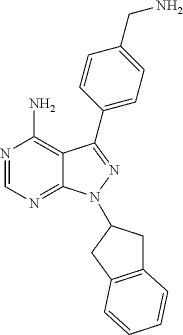 [SMILES: NCc1ccc(-c2nn(C3Cc4ccccc4C3)c3ncnc(N)c23)cc1] | 138 |
| 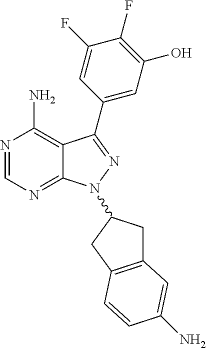 [SMILES: Nc1ccc2c(c1)CC(n1nc(-c3cc(O)c(F)c(F)c3)c3c(N)ncnc31)C2] | 139 139a, 139b |
| 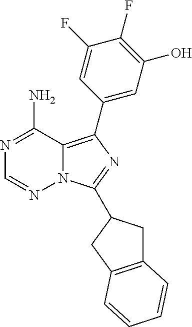 [SMILES: Nc1ncnn2c(C3Cc4ccccc4C3)nc(-c3cc(O)c(F)c(F)c3)c12] | 140 |

TABLE 2
Compounds of the Application

| | |
|---|---|
| 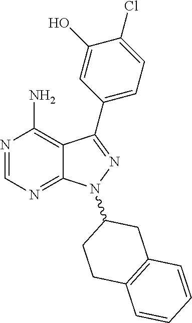 [SMILES: Nc1ncnc2c1c(-c1ccc(Cl)c(O)c1)nn2C1CCc2ccccc2C1] | 2.1 2.1a, 2.1b |
| 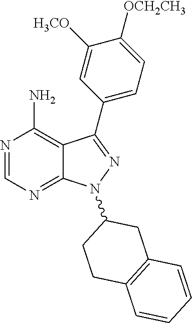 [SMILES: CCOc1ccc(-c2nn(C3CCc4ccccc4C3)c3ncnc(N)c23)cc1OC] | 2.2 2.2a, 2.2b |
| 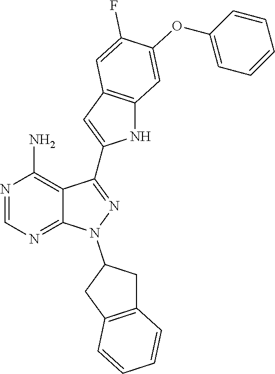 [SMILES: Nc1ncnc2c1c(-c1cc3cc(F)c(Oc4ccccc4)cc3[nH]1)nn2C1Cc2ccccc2C1] | 2.3 |
| 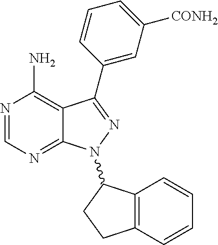 [SMILES: NC(=O)c1cccc(-c2nn(C3CCc4ccccc43)c3ncnc(N)c23)c1] | 2.4 2.4a, 2.4b |
| 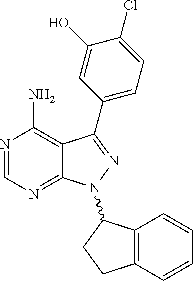 [SMILES: Nc1ncnc2c1c(-c1ccc(Cl)c(O)c1)nn2C1CCc2ccccc21] | 2.5 2.5a, 2.5b |
| 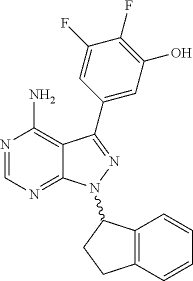 [SMILES: Nc1ncnc2c1c(-c1cc(O)c(F)c(F)c1)nn2C1CCc2ccccc21] | 2.6 2.6a, 2.6b |
| 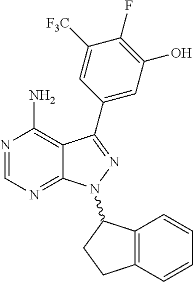 [SMILES: Nc1ncnc2c1c(-c1cc(O)c(F)c(C(F)(F)F)c1)nn2C1CCc2ccccc21] | 2.7 2.7a, 2.7b |
| 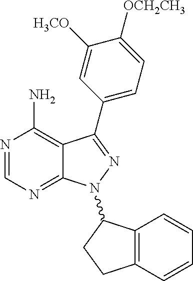 [SMILES: CCOc1ccc(-c2nn(C3CCc4ccccc43)c3ncnc(N)c23)cc1OC] | 2.8 2.8a, 2.8b |
| 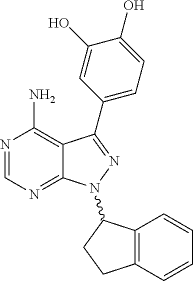 [SMILES: Nc1ncnc2c1c(-c1ccc(O)c(O)c1)nn2C1CCc2ccccc21] | 2.9 2.9a, 2.9b |
| 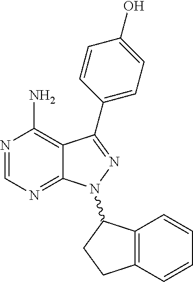 [SMILES: Nc1ncnc2c1c(-c1ccc(O)cc1)nn2C1CCc2ccccc21] | 2.10 2.10a, 2.10b |
| 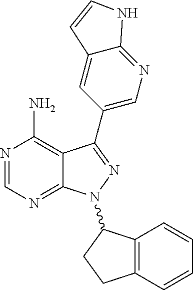 [SMILES: Nc1ncnc2c1c(-c1cnc3[nH]ccc3c1)nn2C1CCc2ccccc21] | 2.11 2.11a, 2.11b |
| 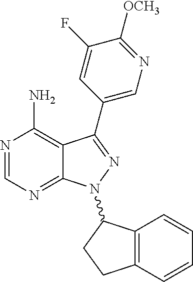 [SMILES: COc1ncc(-c2nn(C3CCc4ccccc43)c3ncnc(N)c23)cc1F] | 2.12 2.12a, 2.12b |
| 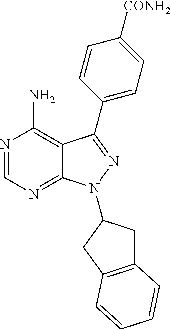 [SMILES: NC(=O)c1ccc(-c2nn(C3Cc4ccccc4C3)c3ncnc(N)c23)cc1] | 2.13 |
| 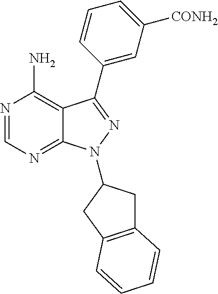 [SMILES: NC(=O)c1cccc(-c2nn(C3Cc4ccccc4C3)c3ncnc(N)c23)c1] | 2.14 |
| 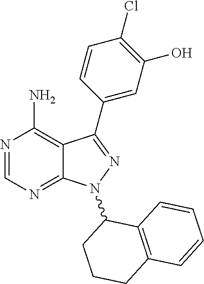 [SMILES: Nc1ncnc2c1c(-c1ccc(Cl)c(O)c1)nn2C1CCCc2ccccc21] | 2.15 2.15a, 2.15b |
| 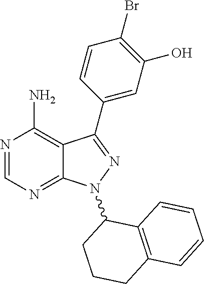 [SMILES: Nc1ncnc2c1c(-c1ccc(Br)c(O)c1)nn2C1CCCc2ccccc21] | 2.16 2.16a, 2.16b |
| 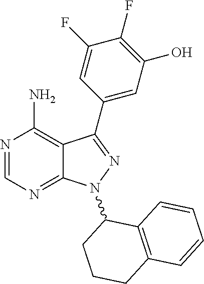 [SMILES: Nc1ncnc2c1c(-c1cc(O)c(F)c(F)c1)nn2C1CCCc2ccccc21] | 2.17 2.17a, 2.17b |
| 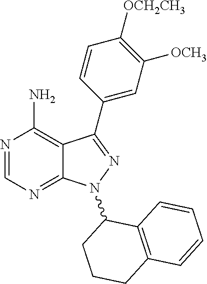 [SMILES: CCOc1ccc(-c2nn(C3CCCc4ccccc43)c3ncnc(N)c23)cc1OC] | 2.18 2.18a, 2.18b |
| 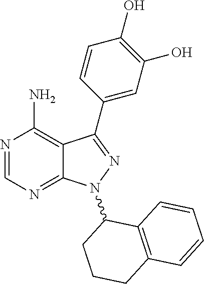 [SMILES: Nc1ncnc2c1c(-c1ccc(O)c(O)c1)nn2C1CCCc2ccccc21] | 2.19 2.19a, 2.19b |
| 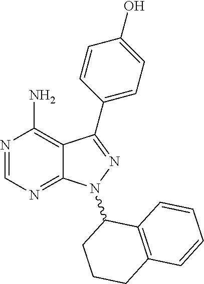 [SMILES: Nc1ncnc2c1c(-c1ccc(O)cc1)nn2C1CCCc2ccccc21] | 2.20 2.20a, 2.20b |
| 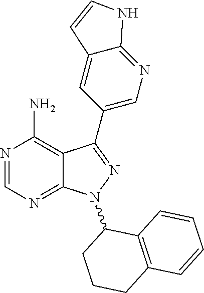 [SMILES: Nc1ncnc2c1c(-c1cnc3[nH]ccc3c1)nn2C1CCCc2ccccc21] | 2.21 2.21a, 2.21b |
| 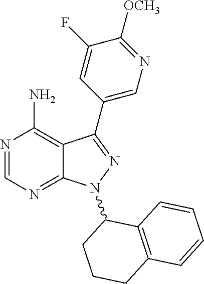 [SMILES: COc1ncc(-c2nn(C3CCCc4ccccc43)c3ncnc(N)c23)cc1F] | 2.22 2.22a, 2.22b |
| 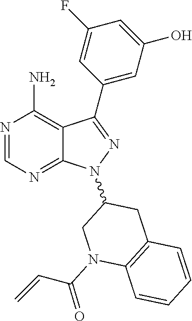 [SMILES: C=CC(=O)N1CC(n2nc(-c3cc(O)cc(F)c3)c3c(N)ncnc32)Cc2ccccc21] | 2.23 2.23a, 2.23b |
| 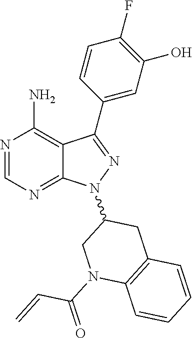 [SMILES: C=CC(=O)N1CC(n2nc(-c3ccc(F)c(O)c3)c3c(N)ncnc32)Cc2ccccc21] | 2.24 2.24a, 2.24b |
| 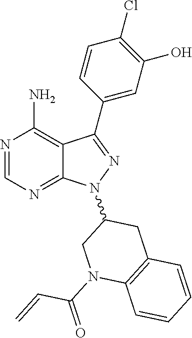 [SMILES: C=CC(=O)N1CC(n2nc(-c3ccc(Cl)c(O)c3)c3c(N)ncnc32)Cc2ccccc21] | 2.25 2.25a, 2.25b |
| 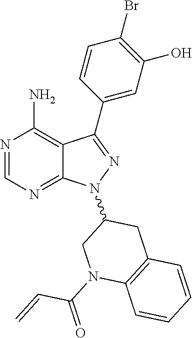 [SMILES: C=CC(=O)N1CC(n2nc(-c3ccc(Br)c(O)c3)c3c(N)ncnc32)Cc2ccccc21] | 2.26 2.26a, 2.26b |
| 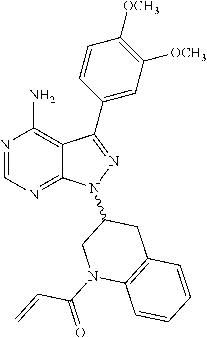 [SMILES: C=CC(=O)N1CC(n2nc(-c3ccc(OC)c(OC)c3)c3c(N)ncnc32)Cc2ccccc21] | 2.27 2.27a, 2.27b |
| 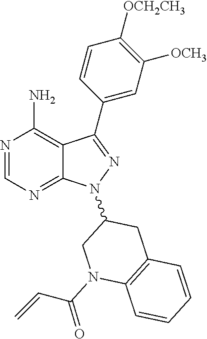 [SMILES: C=CC(=O)N1CC(n2nc(-c3ccc(OCC)c(OC)c3)c3c(N)ncnc32)Cc2ccccc21] | 2.28 2.28a, 2.28b |
| 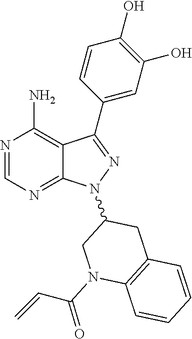 [SMILES: C=CC(=O)N1CC(n2nc(-c3ccc(O)c(O)c3)c3c(N)ncnc32)Cc2ccccc21] | 2.29 2.29a, 2.29b |
| 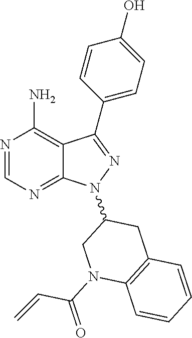 [SMILES: C=CC(=O)N1CC(n2nc(-c3ccc(O)cc3)c3c(N)ncnc32)Cc2ccccc21] | 2.30 2.30a, 2.30b |
| 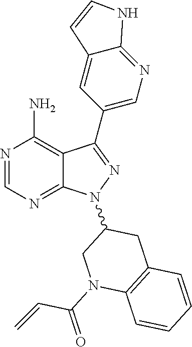 [SMILES: C=CC(=O)N1CC(n2nc(-c3cnc4[nH]ccc4c3)c3c(N)ncnc32)Cc2ccccc21] | 2.31 2.31a, 2.31b |
| 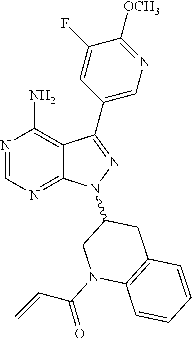 [SMILES: C=CC(=O)N1CC(n2nc(-c3cnc(OC)c(F)c3)c3c(N)ncnc32)Cc2ccccc21] | 2.32 2.32a, 2.32b |
| 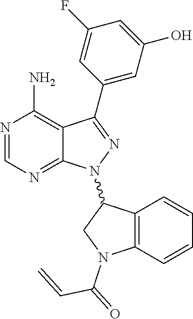 [SMILES: C=CC(=O)N1CC(n2nc(-c3cc(O)cc(F)c3)c3c(N)ncnc32)c2ccccc21] | 2.33 2.33a, 2.33b |
| 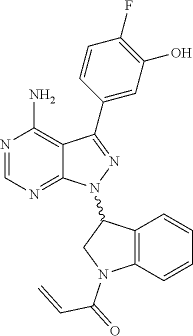 [SMILES: C=CC(=O)N1CC(n2nc(-c3ccc(F)c(O)c3)c3c(N)ncnc32)c2ccccc21] | 2.34 2.34a, 2.34b |
| 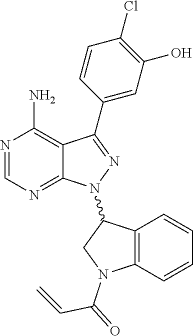 [SMILES: C=CC(=O)N1CC(n2nc(-c3ccc(Cl)c(O)c3)c3c(N)ncnc32)c2ccccc21] | 2.35 2.35a, 2.35b |
| 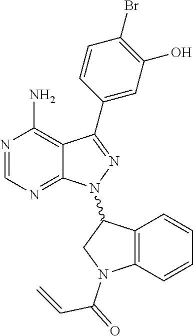 [SMILES: C=CC(=O)N1CC(n2nc(-c3ccc(Br)c(O)c3)c3c(N)ncnc32)c2ccccc21] | 2.36 2.36a, 2.36b |
| 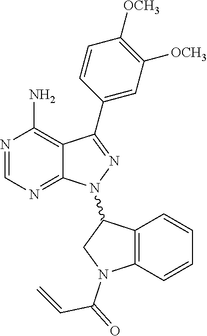 [SMILES: C=CC(=O)N1CC(n2nc(-c3ccc(OC)c(OC)c3)c3c(N)ncnc32)c2ccccc21] | 2.37 2.37a, 2.37b |
| 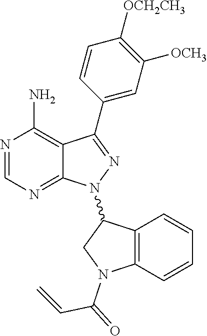 [SMILES: C=CC(=O)N1CC(n2nc(-c3ccc(OCC)c(OC)c3)c3c(N)ncnc32)c2ccccc21] | 2.38 2.38a, 2.38b |
| 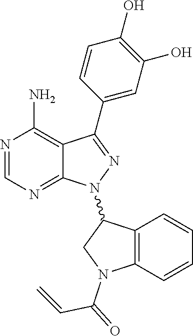 [SMILES: C=CC(=O)N1CC(n2nc(-c3ccc(O)c(O)c3)c3c(N)ncnc32)c2ccccc21] | 2.39 2.39a, 2.39b |
| 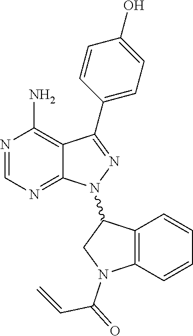 [SMILES: C=CC(=O)N1CC(n2nc(-c3ccc(O)cc3)c3c(N)ncnc32)c2ccccc21] | 2.40 2.40a, 2.40b |
| 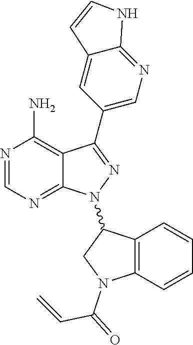 [SMILES: C=CC(=O)N1CC(n2nc(-c3cnc4[nH]ccc4c3)c3c(N)ncnc32)c2ccccc21] | 2.41 2.41a, 2.41b |
| 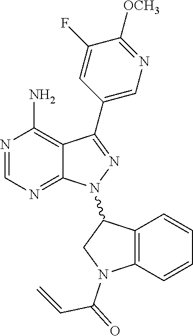 [SMILES: C=CC(=O)N1CC(n2nc(-c3cnc(OC)c(F)c3)c3c(N)ncnc32)c2ccccc21] | 2.42 2.42a, 2.42b |
| 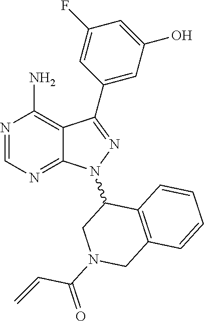 [SMILES: C=CC(=O)N1Cc2ccccc2C(n2nc(-c3cc(O)cc(F)c3)c3c(N)ncnc32)C1] | 2.43 2.43a, 2.43b |
| 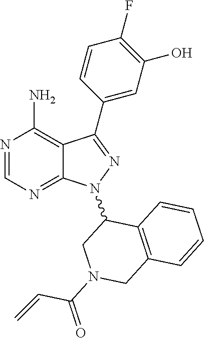 [SMILES: C=CC(=O)N1Cc2ccccc2C(n2nc(-c3ccc(F)c(O)c3)c3c(N)ncnc32)C1] | 2.44 2.44a, 2.44b |
| 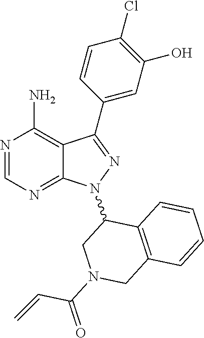 [SMILES: C=CC(=O)N1Cc2ccccc2C(n2nc(-c3ccc(Cl)c(O)c3)c3c(N)ncnc32)C1] | 2.45 2.45a, 2.45b |
| 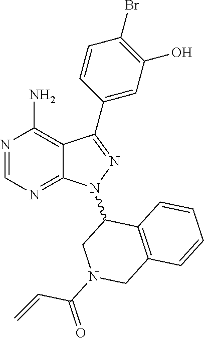 [SMILES: C=CC(=O)N1Cc2ccccc2C(n2nc(-c3ccc(Br)c(O)c3)c3c(N)ncnc32)C1] | 2.46 2.46a, 2.46b |
| 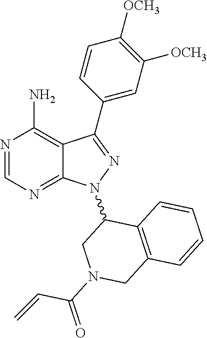 [SMILES: C=CC(=O)N1Cc2ccccc2C(n2nc(-c3ccc(OC)c(OC)c3)c3c(N)ncnc32)C1] | 2.47 2.47a, 2.47b |
| 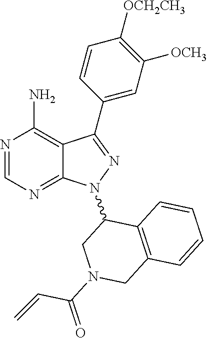 [SMILES: C=CC(=O)N1Cc2ccccc2C(n2nc(-c3ccc(OCC)c(OC)c3)c3c(N)ncnc32)C1] | 2.48 2.48a, 2.48b |
| 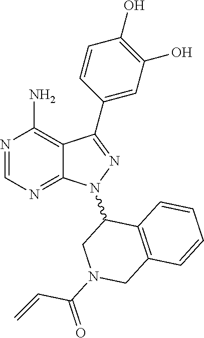 [SMILES: C=CC(=O)N1Cc2ccccc2C(n2nc(-c3ccc(O)c(O)c3)c3c(N)ncnc32)C1] | 2.49 2.49a, 2.49b |
| 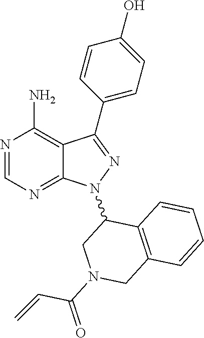 [SMILES: C=CC(=O)N1Cc2ccccc2C(n2nc(-c3ccc(O)cc3)c3c(N)ncnc32)C1] | 2.50 2.50a, 2.50b |
| 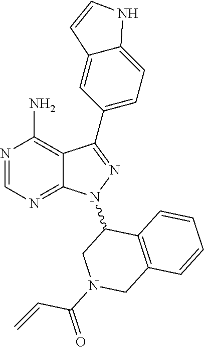 [SMILES: C=CC(=O)N1Cc2ccccc2C(n2nc(-c3ccc4[nH]ccc4c3)c3c(N)ncnc32)C1] | 2.51 2.51a, 2.51b |
| 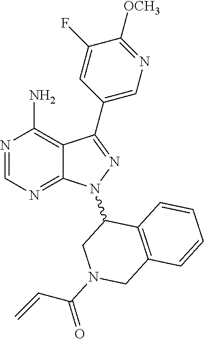 [SMILES: C=CC(=O)N1Cc2ccccc2C(n2nc(-c3cnc(OC)c(F)c3)c3c(N)ncnc32)C1] | 2.52 2.52a, 2.52b |
| 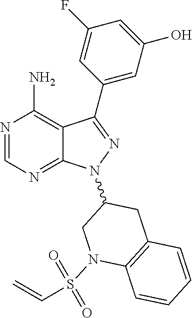 [SMILES: C=CS(=O)(=O)N1CC(n2nc(-c3cc(O)cc(F)c3)c3c(N)ncnc32)Cc2ccccc21] | 2.53 2.53a, 2.53b |
| 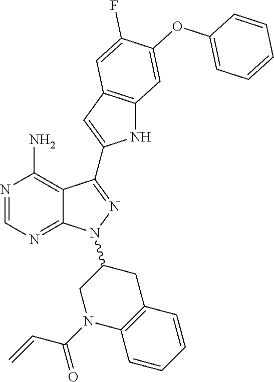 [SMILES: C=CC(=O)N1CC(n2nc(-c3cc4cc(F)c(Oc5ccccc5)cc4[nH]3)c3c(N)ncnc32)Cc2ccccc21] | 2.54 2.54a, 2.54b |
| 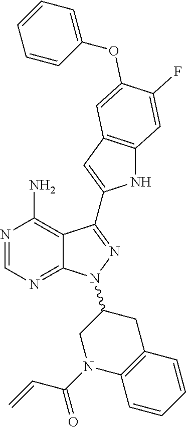 [SMILES: C=CC(=O)N1CC(n2nc(-c3cc4cc(Oc5ccccc5)c(F)cc4[nH]3)c3c(N)ncnc32)Cc2ccccc21] | 2.55 2.55a, 2.55b |
| 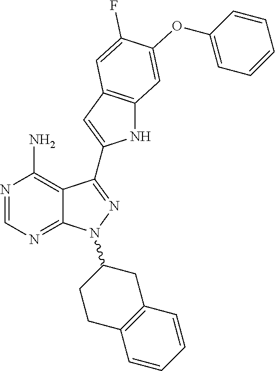 [SMILES: Nc1ncnc2c1c(-c1cc3cc(F)c(Oc4ccccc4)cc3[nH]1)nn2C1CCc2ccccc2C1] | 2.56 2.56a, 2.56b |
| 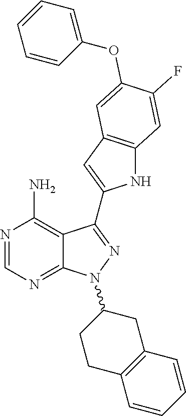 [SMILES: Nc1ncnc2c1c(-c1cc3cc(Oc4ccccc4)c(F)cc3[nH]1)nn2C1CCc2ccccc2C1] | 2.57 2.57a, 2.57b |
| 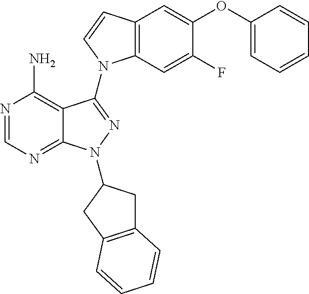 [SMILES: Nc1ncnc2c1c(-n1ccc3cc(Oc4ccccc4)c(F)cc31)nn2C1Cc2ccccc2C1] | 2.58 |
| 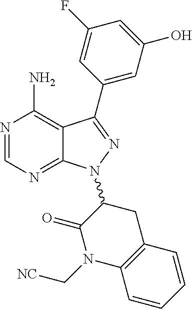 [SMILES: N#CCN1C(=O)C(n2nc(-c3cc(O)cc(F)c3)c3c(N)ncnc32)Cc2ccccc21] | 2.59 2.59a, 2.59b |
| 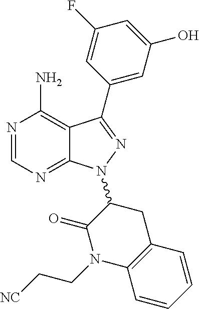 [SMILES: N#CCCN1C(=O)C(n2nc(-c3cc(O)cc(F)c3)c3c(N)ncnc32)Cc2ccccc21] | 2.60 2.60a, 2.60b |
| 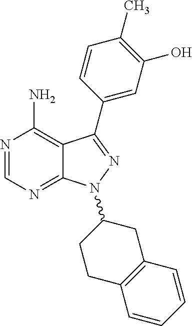 [SMILES: Cc1ccc(-c2nn(C3CCc4ccccc4C3)c3ncnc(N)c23)cc1O] | 2.61 2.61a, 2.61b |
| 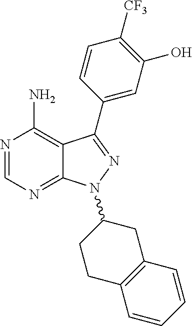 [SMILES: Nc1ncnc2c1c(-c1ccc(C(F)(F)F)c(O)c1)nn2C1CCc2ccccc2C1] | 2.62 2.62a, 2.62b |
| 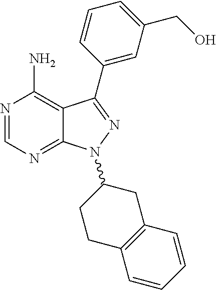 [SMILES: Nc1ncnc2c1c(-c1cccc(CO)c1)nn2C1CCc2ccccc2C1] | 2.63 2.63a, 2.63b |
| 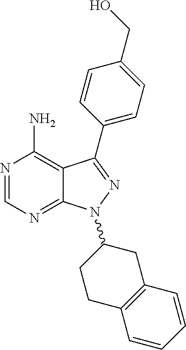 [SMILES: Nc1ncnc2c1c(-c1ccc(CO)cc1)nn2C1CCc2ccccc2C1] | 2.64 2.64a, 2.64b |
| 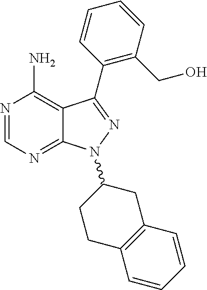 [SMILES: Nc1ncnc2c1c(-c1ccccc1CO)nn2C1CCc2ccccc2C1] | 2.65 2.65a, 2.65b |
| 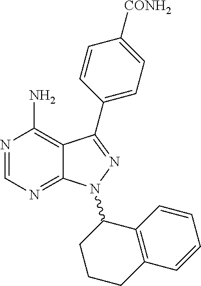 [SMILES: NC(=O)c1ccc(-c2nn(C3CCCc4ccccc43)c3ncnc(N)c23)cc1] | 2.66 2.66a, 2.66b |
| 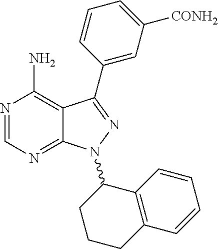 [SMILES: NC(=O)c1cccc(-c2nn(C3CCCc4ccccc43)c3ncnc(N)c23)c1] | 2.67 2.67a, 2.67b |
| 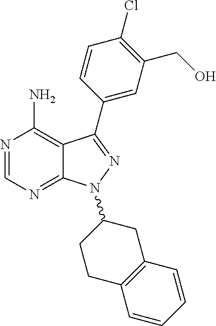 [SMILES: Nc1ncnc2c1c(-c1ccc(Cl)c(CO)c1)nn2C1CCc2ccccc2C1] | 2.68 2.68a, 2.68b |
| 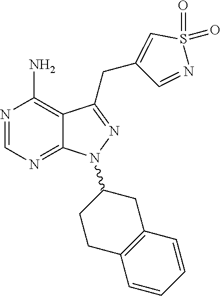 [SMILES: Nc1ncnc2c1c(CC1=CS(=O)(=O)N=C1)nn2C1CCc2ccccc2C1] | 2.69 2.69a, 2.69b |
| 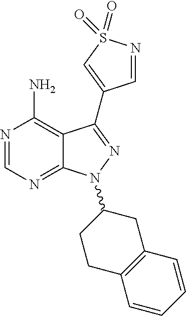 [SMILES: Nc1ncnc2c1c(C1=CS(=O)(=O)N=C1)nn2C1CCc2ccccc2C1] | 2.70 2.70a, 2.70b |
| 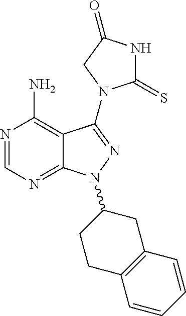 [SMILES: Nc1ncnc2c1c(N1CC(=O)NC1=S)nn2C1CCc2ccccc2C1] | 2.71 2.71a, 2.71b |
| 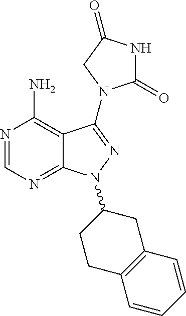 [SMILES: Nc1ncnc2c1c(N1CC(=O)NC1=O)nn2C1CCc2ccccc2C1] | 2.72 2.72a, 2.72b |
| 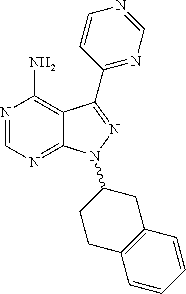 [SMILES: Nc1ncnc2c1c(-c1ccncn1)nn2C1CCc2ccccc2C1] | 2.73 2.73a, 2.73b |
| 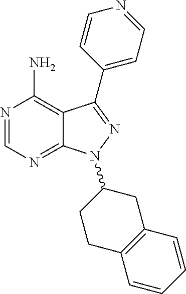 [SMILES: Nc1ncnc2c1c(-c1ccncc1)nn2C1CCc2ccccc2C1] | 2.74 2.74a, 2.74b |
| 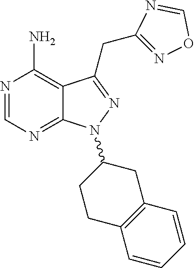 [SMILES: Nc1ncnc2c1c(Cc1ncon1)nn2C1CCc2ccccc2C1] | 2.75 2.75a, 2.75b |
| 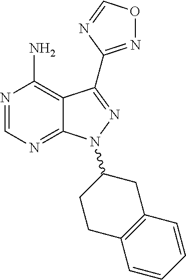 [SMILES: Nc1ncnc2c1c(-c1ncon1)nn2C1CCc2ccccc2C1] | 2.76 2.76a, 2.76b |
| 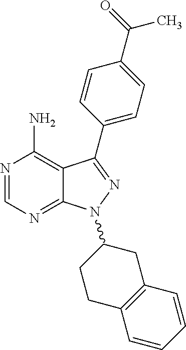 [SMILES: CC(=O)c1ccc(-c2nn(C3CCc4ccccc4C3)c3ncnc(N)c23)cc1] | 2.77 2.77a, 2.77b |
| 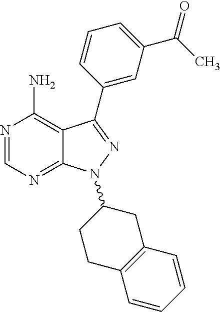 [SMILES: CC(=O)c1cccc(-c2nn(C3CCc4ccccc4C3)c3ncnc(N)c23)c1] | 2.78 2.78a, 2.78b |
| 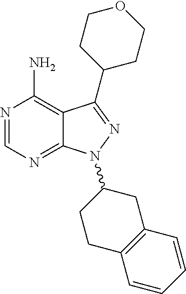 [SMILES: Nc1ncnc2c1c(C1CCOCC1)nn2C1CCc2ccccc2C1] | 2.79 2.79a, 2.79b |
| 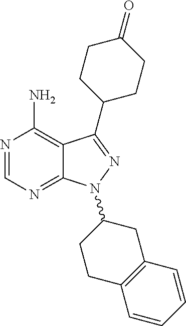 [SMILES: Nc1ncnc2c1c(C1CCC(=O)CC1)nn2C1CCc2ccccc2C1] | 2.80 2.80a, 2.80b |
| 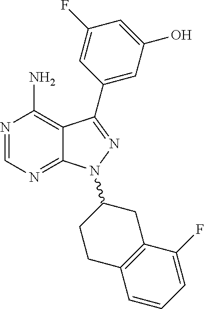 [SMILES: Nc1ncnc2c1c(-c1cc(O)cc(F)c1)nn2C1CCc2cccc(F)c2C1] | 2.81 2.81a, 2.81b |
| 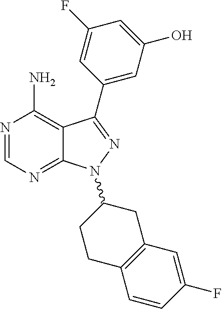 [SMILES: Nc1ncnc2c1c(-c1cc(O)cc(F)c1)nn2C1CCc2ccc(F)cc2C1] | 2.82 2.82a, 2.82b |
| 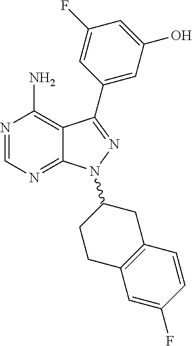 [SMILES: Nc1ncnc2c1c(-c1cc(O)cc(F)c1)nn2C1CCc2cc(F)ccc2C1] | 2.83 2.83a, 2.83b |
| 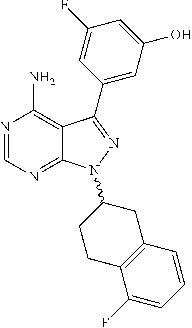 [SMILES: Nc1ncnc2c1c(-c1cc(O)cc(F)c1)nn2C1CCc2c(F)cccc2C1] | 2.84 2.84a, 2.84b |
| 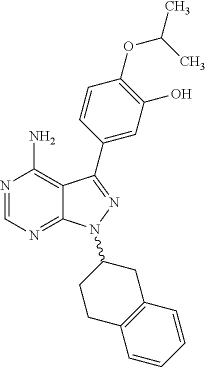 [SMILES: CC(C)Oc1ccc(-c2nn(C3CCc4ccccc4C3)c3ncnc(N)c23)cc1O] | 2.85 2.85a, 2.85b |
| 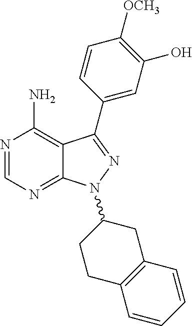 [SMILES: COc1ccc(-c2nn(C3CCc4ccccc4C3)c3ncnc(N)c23)cc1O] | 2.86 2.86a, 2.86b |
| 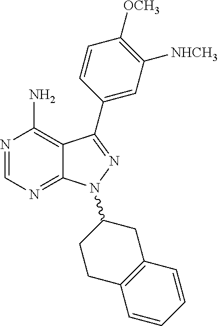 [SMILES: CNc1cc(-c2nn(C3CCc4ccccc4C3)c3ncnc(N)c23)ccc1OC] | 2.87 2.87a, 2.87b |
| 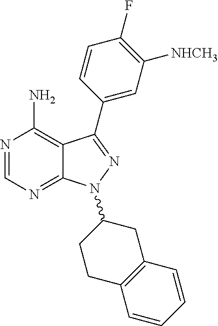 [SMILES: CNc1cc(-c2nn(C3CCc4ccccc4C3)c3ncnc(N)c23)ccc1F] | 2.88 2.88a, 2.88b |
| 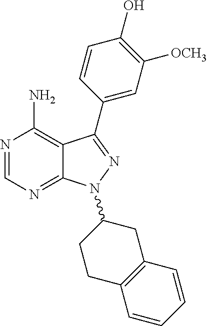 [SMILES: COc1cc(-c2nn(C3CCc4ccccc4C3)c3ncnc(N)c23)ccc1O] | 2.89 2.89a, 2.89b |
| 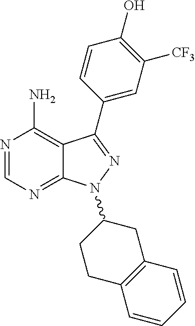 [SMILES: Nc1ncnc2c1c(-c1ccc(O)c(C(F)(F)F)c1)nn2C1CCc2ccccc2C1] | 2.90 2.90a, 2.90b |
| 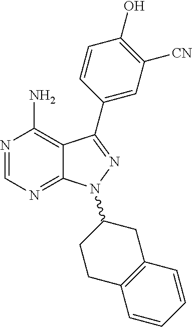 [SMILES: N#Cc1cc(-c2nn(C3CCc4ccccc4C3)c3ncnc(N)c23)ccc1O] | 2.91 2.91a, 2.91b |
| 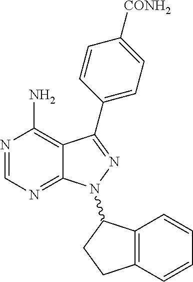 [SMILES: NC(=O)c1ccc(-c2nn(C3CCc4ccccc43)c3ncnc(N)c23)cc1] | 2.92 2.92a, 2.92b |
| 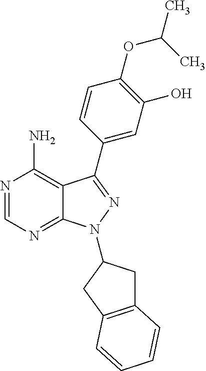 [SMILES: CC(C)Oc1ccc(-c2nn(C3Cc4ccccc4C3)c3ncnc(N)c23)cc1O] | 2.93 |
| 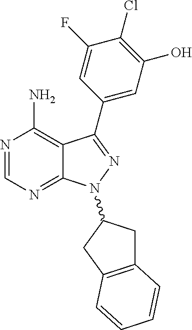 [SMILES: Nc1ncnc2c1c(-c1cc(O)c(Cl)c(F)c1)nn2C1Cc2ccccc2C1] | 2.94 |
| 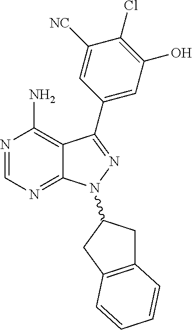 [SMILES: N#Cc1cc(-c2nn(C3Cc4ccccc4C3)c3ncnc(N)c23)cc(O)c1Cl] | 2.95 |
| 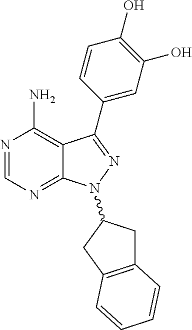 [SMILES: Nc1ncnc2c1c(-c1ccc(O)c(O)c1)nn2C1Cc2ccccc2C1] | 2.96 |
| 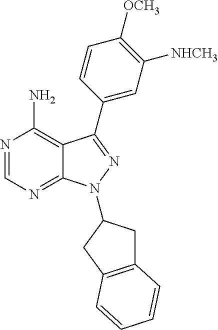 [SMILES: CNc1cc(-c2nn(C3Cc4ccccc4C3)c3ncnc(N)c23)ccc1OC] | 2.97 |
| 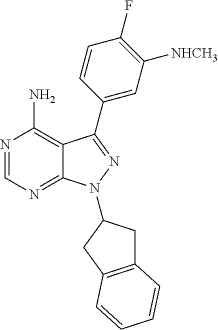 [SMILES: CNc1cc(-c2nn(C3Cc4ccccc4C3)c3ncnc(N)c23)ccc1F] | 2.98 |
| 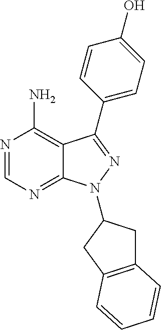 [SMILES: Nc1ncnc2c1c(-c1ccc(O)cc1)nn2C1Cc2ccccc2C1] | 2.99 |
| 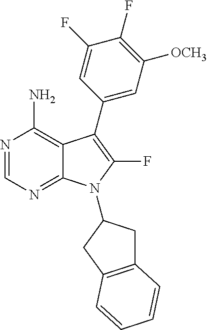 [SMILES: COc1cc(-c2c(F)n(C3Cc4ccccc4C3)c3ncnc(N)c23)cc(F)c1F] | 2.100 |
| 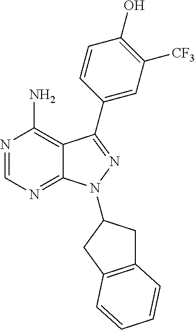 [SMILES: Nc1ncnc2c1c(-c1ccc(O)c(C(F)(F)F)c1)nn2C1Cc2ccccc2C1] | 2.101 |
| 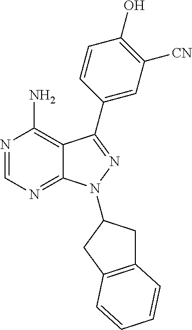 [SMILES: N#Cc1cc(-c2nn(C3Cc4ccccc4C3)c3ncnc(N)c23)ccc1O] | 2.102 |
| 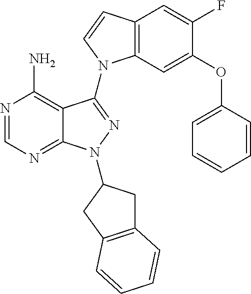 [SMILES: Nc1ncnc2c1c(-n1ccc3cc(F)c(Oc4ccccc4)cc31)nn2C1Cc2ccccc2C1] | 2.103 |
| 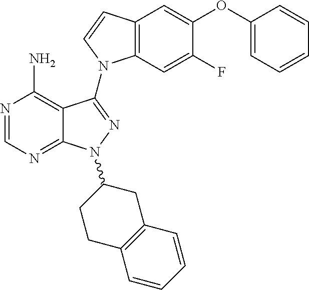 [SMILES: Nc1ncnc2c1c(-n1ccc3cc(Oc4ccccc4)c(F)cc31)nn2C1CCc2ccccc2C1] | 2.104 2.104a, 2.104b |
| 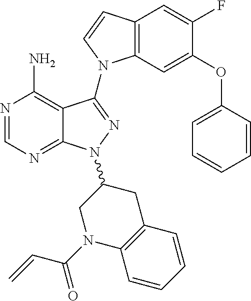 [SMILES: C=CC(=O)N1CC(n2nc(-n3ccc4cc(F)c(Oc5ccccc5)cc43)c3c(N)ncnc32)Cc2ccccc21] | 2.105 2.105a, 2.105b |
| 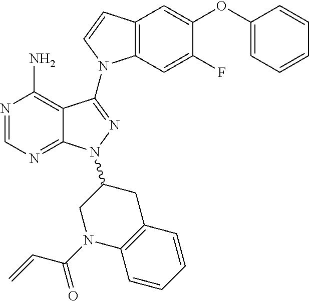 [SMILES: C=CC(=O)N1CC(n2nc(-n3ccc4cc(Oc5ccccc5)c(F)cc43)c3c(N)ncnc32)Cc2ccccc21] | 2.106 2.106a, 2.106b |
| 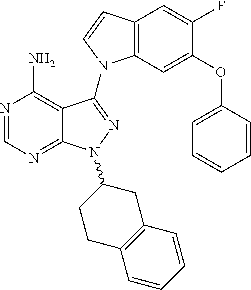 [SMILES: Nc1ncnc2c1c(-n1ccc3cc(F)c(Oc4ccccc4)cc31)nn2C1CCc2ccccc2C1] | 2.107 2.107a, 2.107b |
| 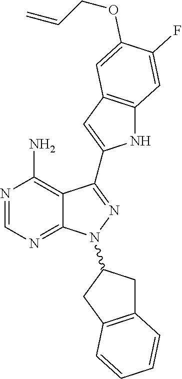 [SMILES: C=CCOc1cc2cc(-c3nn(C4Cc5ccccc5C4)c4ncnc(N)c34)[nH]c2cc1F] | 2.108 |
| 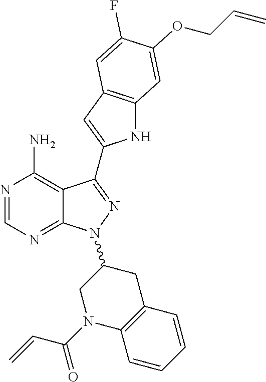 [SMILES: C=CCOc1cc2[nH]c(-c3nn(C4Cc5ccccc5N(C(=O)C=C)C4)c4ncnc(N)c34)cc2cc1F] | 2.109 2.109a, 2.109b |
| 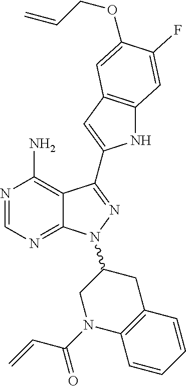 [SMILES: C=CCOc1cc2cc(-c3nn(C4Cc5ccccc5N(C(=O)C=C)C4)c4ncnc(N)c34)[nH]c2cc1F] | 2.110 2.110a, 2.110b |
| 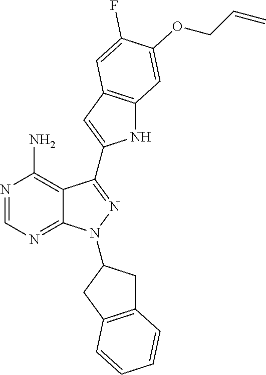 [SMILES: C=CCOc1cc2[nH]c(-c3nn(C4Cc5ccccc5C4)c4ncnc(N)c34)cc2cc1F] | 2.111 |
| 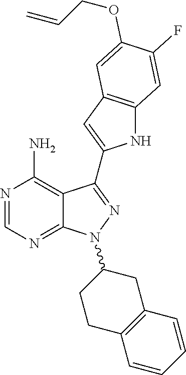 [SMILES: C=CCOc1cc2cc(-c3nn(C4CCc5ccccc5C4)c4ncnc(N)c34)[nH]c2cc1F] | 2.112 2.112a, 2.112b |
| 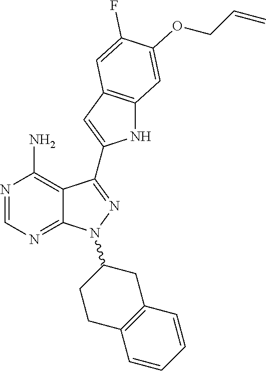 [SMILES: C=CCOc1cc2[nH]c(-c3nn(C4CCc5ccccc5C4)c4ncnc(N)c34)cc2cc1F] | 2.113 2.113a, 2.113b |
| 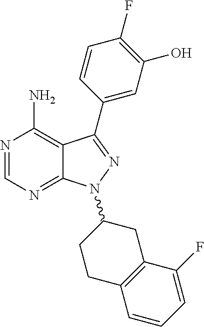 [SMILES: Nc1ncnc2c1c(-c1ccc(F)c(O)c1)nn2C1CCc2cccc(F)c2C1] | 2.114 2.114a, 2.114b |
| 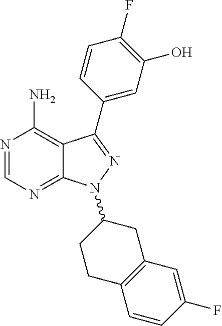 [SMILES: Nc1ncnc2c1c(-c1ccc(F)c(O)c1)nn2C1CCc2ccc(F)cc2C1] | 2.115 2.115a, 2.115b |
| 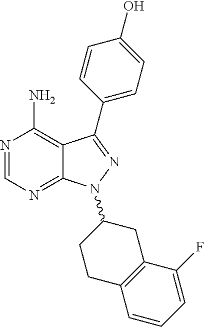 [SMILES: Nc1ncnc2c1c(-c1ccc(O)cc1)nn2C1CCc2cccc(F)c2C1] | 2.116 2.116a, 2.116b |
| 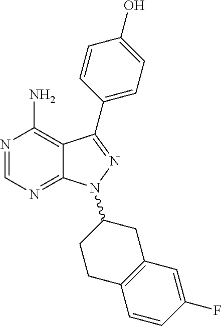 [SMILES: Nc1ncnc2c1c(-c1ccc(O)cc1)nn2C1CCc2ccc(F)cc2C1] | 2.117 2.117a, 2.117b |
| 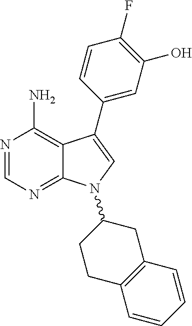 [SMILES: Nc1ncnc2c1c(-c1ccc(F)c(O)c1)cn2C1CCc2ccccc2C1] | 2.118 2.118a, 2.118b |
| 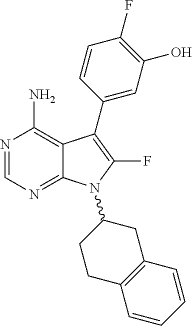 [SMILES: Nc1ncnc2c1c(-c1ccc(F)c(O)c1)c(F)n2C1CCc2ccccc2C1] | 2.119 2.119a, 2.119b |
| 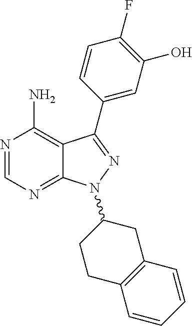 [SMILES: Nc1ncnc2c1c(-c1ccc(F)c(O)c1)nn2C1CCc2ccccc2C1] | 2.120 2.120a, 2.120b |
| 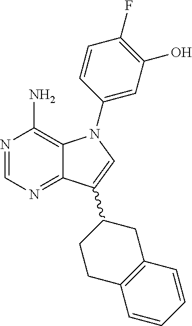 [SMILES: Nc1ncnc2c(C3CCc4ccccc4C3)cn(-c3ccc(F)c(O)c3)c12] | 2.121 2.121a, 2.121b |
| 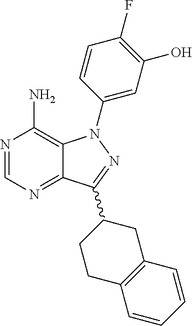 [SMILES: Nc1ncnc2c(C3CCc4ccccc4C3)nn(-c3ccc(F)c(O)c3)c12] | 2.122 2.122a, 2.122b |
| 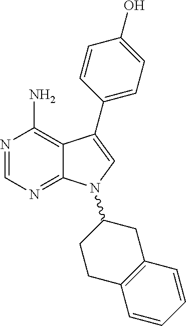 [SMILES: Nc1ncnc2c1c(-c1ccc(O)cc1)cn2C1CCc2ccccc2C1] | 2.123 2.123a, 2.123b |
| 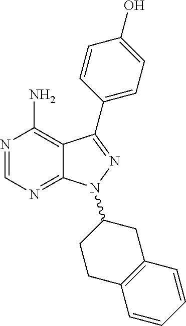 [SMILES: Nc1ncnc2c1c(-c1ccc(O)cc1)nn2C1CCc2ccccc2C1] | 2.124 2.124a, 2.124b |
| 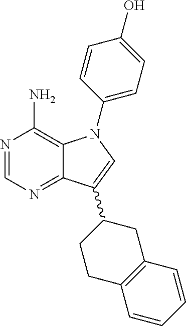 [SMILES: Nc1ncnc2c(C3CCc4ccccc4C3)cn(-c3ccc(O)cc3)c12] | 2.125 2.125a, 2.125b |
| 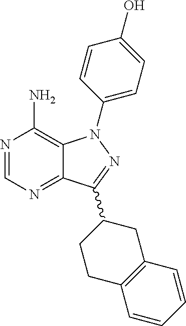 [SMILES: Nc1ncnc2c(C3CCc4ccccc4C3)nn(-c3ccc(O)cc3)c12] | 2.126 2.126a, 2.126b |
| 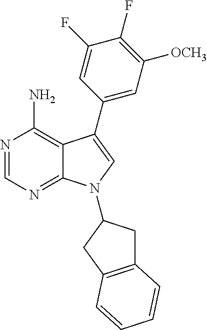 [SMILES: COc1cc(-c2cn(C3Cc4ccccc4C3)c3ncnc(N)c23)cc(F)c1F] | 2.127 |
| 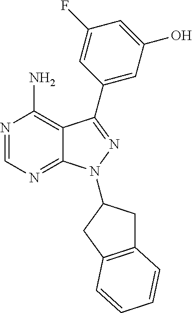 [SMILES: Nc1ncnc2c1c(-c1cc(O)cc(F)c1)nn2C1Cc2ccccc2C1] | 2.128 |
| 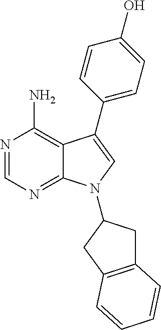 [SMILES: Nc1ncnc2c1c(-c1ccc(O)cc1)cn2C1Cc2ccccc2C1] | 2.129 |
| 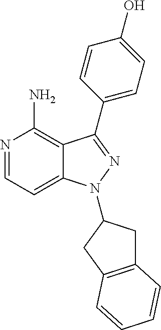 [SMILES: Nc1nccc2c1c(-c1ccc(O)cc1)nn2C1Cc2ccccc2C1] | 2.130 |
| 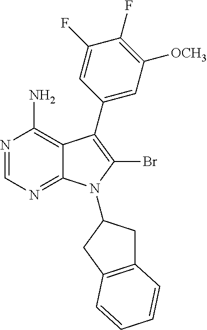 [SMILES: COc1cc(-c2c(Br)n(C3Cc4ccccc4C3)c3ncnc(N)c23)cc(F)c1F] | 2.131 |
| 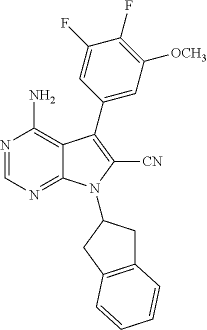 [SMILES: COc1cc(-c2c(C#N)n(C3Cc4ccccc4C3)c3ncnc(N)c23)cc(F)c1F] | 2.132 |
| 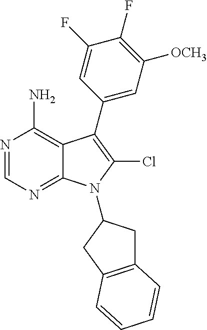 [SMILES: COc1cc(-c2c(Cl)n(C3Cc4ccccc4C3)c3ncnc(N)c23)cc(F)c1F] | 2.133 |
| 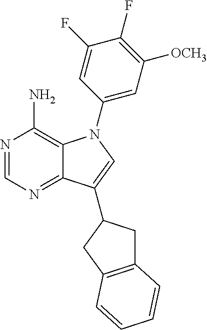 [SMILES: COc1cc(-n2cc(C3Cc4ccccc4C3)c3ncnc(N)c32)cc(F)c1F] | 2.134 |
| 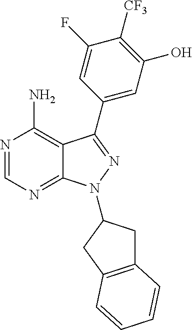 [SMILES: Nc1ncnc2c1c(-c1cc(O)c(C(F)(F)F)c(F)c1)nn2C1Cc2ccccc2C1] | 2.135 |
| 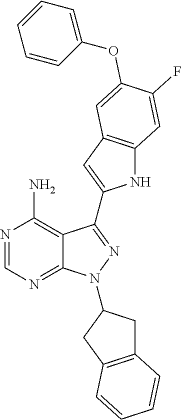 [SMILES: Nc1ncnc2c1c(-c1cc3cc(Oc4ccccc4)c(F)cc3[nH]1)nn2C1Cc2ccccc2C1] | 2.136 |
| 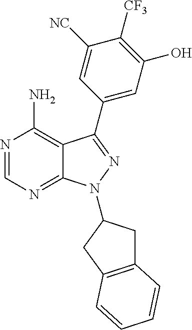 [SMILES: N#Cc1cc(-c2nn(C3Cc4ccccc4C3)c3ncnc(N)c23)cc(O)c1C(F)(F)F] | 2.137 |
| 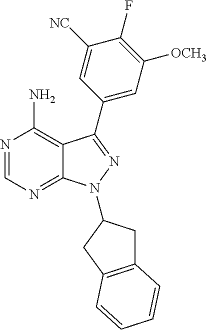 [SMILES: COc1cc(-c2nn(C3Cc4ccccc4C3)c3ncnc(N)c23)cc(C#N)c1F] | 2.138 |
| 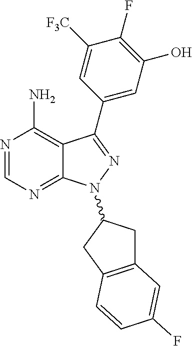 [SMILES: Nc1ncnc2c1c(-c1cc(O)c(F)c(C(F)(F)F)c1)nn2C1Cc2ccc(F)cc2C1] | 2.139 2.139a, 2.139b |
| 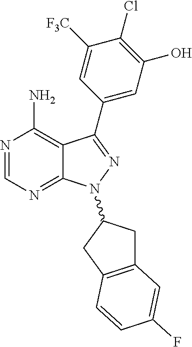 [SMILES: Nc1ncnc2c1c(-c1cc(O)c(Cl)c(C(F)(F)F)c1)nn2C1Cc2ccc(F)cc2C1] | 2.140 2.140a, 2.140b |
| 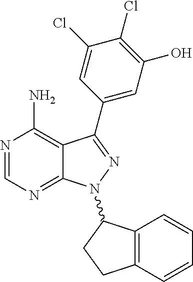 [SMILES: Nc1ncnc2c1c(-c1cc(O)c(Cl)c(Cl)c1)nn2C1CCc2ccccc21] | 2.141 2.141a, 2.141b |
| 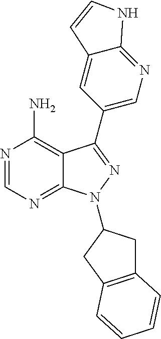 [SMILES: Nc1ncnc2c1c(-c1cnc3[nH]ccc3c1)nn2C1Cc2ccccc2C1] | 2.142 |
| 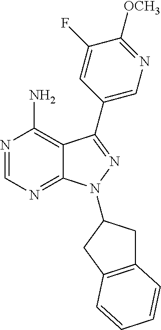 [SMILES: COc1ncc(-c2nn(C3Cc4ccccc4C3)c3ncnc(N)c23)cc1F] | 2.143 |
| 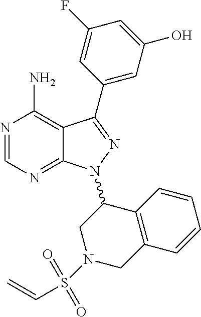 [SMILES: C=CS(=O)(=O)N1Cc2ccccc2C(n2nc(-c3cc(O)cc(F)c3)c3c(N)ncnc32)C1] | 2.144 2.144a, 2.144b |
| 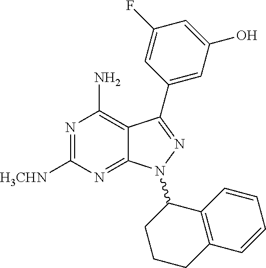 | 2.145 2.145a, 2.145b |
| 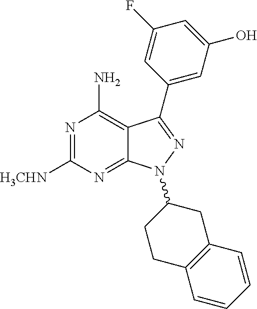 | 2.146 2.146a, 2.146b |
| 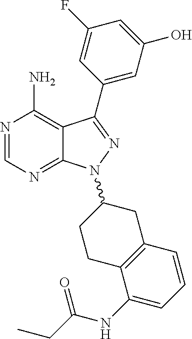 [SMILES: CCC(=O)Nc1cccc2c1CCC(n1nc(-c3cc(O)cc(F)c3)c3c(N)ncnc31)C2] | 2.147 2.147a, 2.147b |
| 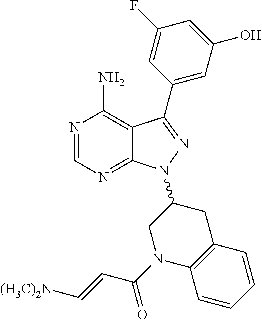 | 2.148 2.148a, 2.148b |
| 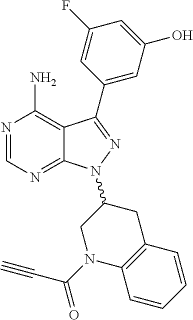 [SMILES: C#CC(=O)N1CC(n2nc(-c3cc(O)cc(F)c3)c3c(N)ncnc32)Cc2ccccc21] | 2.149 2.149a, 2.149b |
| 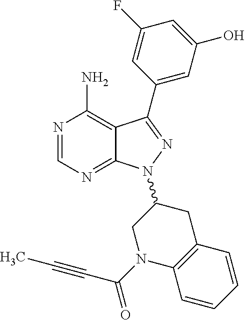 [SMILES: CC#CC(=O)N1CC(n2nc(-c3cc(O)cc(F)c3)c3c(N)ncnc32)Cc2ccccc21] | 2.150 2.150a, 2.150b |
| 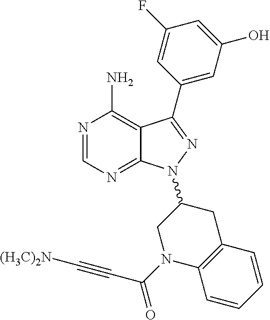 | 2.151 2.151a, 2.151b |
| 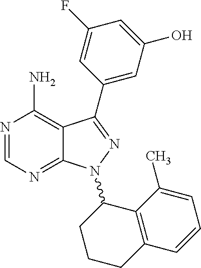 [SMILES: Cc1cccc2c1C(n1nc(-c3cc(O)cc(F)c3)c3c(N)ncnc31)CCC2] | 2.152 2.152a, 2.152b |
| 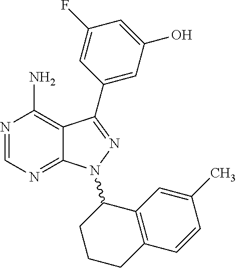 [SMILES: Cc1ccc2c(c1)C(n1nc(-c3cc(O)cc(F)c3)c3c(N)ncnc31)CCC2] | 2.153 2.153a, 2.153b |
| 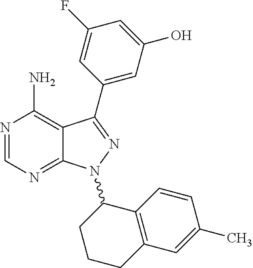 [SMILES: Cc1ccc2c(c1)CCCC2n1nc(-c2cc(O)cc(F)c2)c2c(N)ncnc21] | 2.154 2.154a, 2.154b |
| 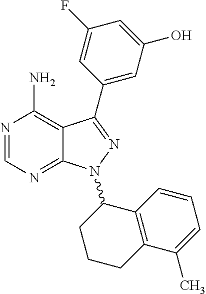 [SMILES: Cc1cccc2c1CCCC2n1nc(-c2cc(O)cc(F)c2)c2c(N)ncnc21] | 2.155 2.155a, 2.155b |
| 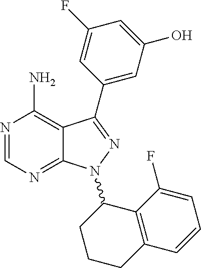 [SMILES: Nc1ncnc2c1c(-c1cc(O)cc(F)c1)nn2C1CCCc2cccc(F)c21] | 2.156 2.156a, 2.156b |
| 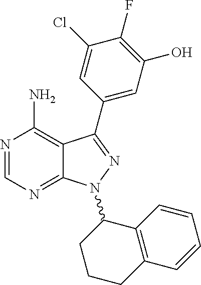 [SMILES: Nc1ncnc2c1c(-c1cc(O)c(F)c(Cl)c1)nn2C1CCCc2ccccc21] | 2.157 2.157a, 2.157b |
| 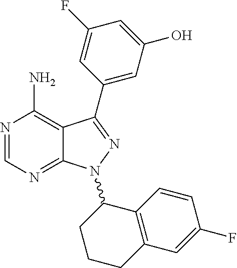 [SMILES: Nc1ncnc2c1c(-c1cc(O)cc(F)c1)nn2C1CCCc2cc(F)ccc21] | 2.158 2.158a, 2.158b |
| 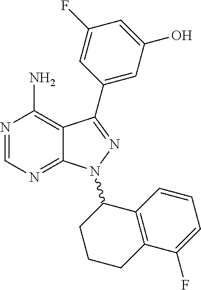 [SMILES: Nc1ncnc2c1c(-c1cc(O)cc(F)c1)nn2C1CCCc2c(F)cccc21] | 2.159 2.159a, 2.159b |
| 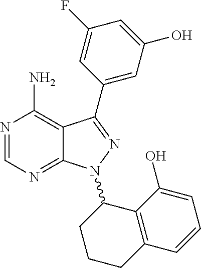 [SMILES: Nc1ncnc2c1c(-c1cc(O)cc(F)c1)nn2C1CCCc2cccc(O)c21] | 2.160 2.160a, 2.160b |
| 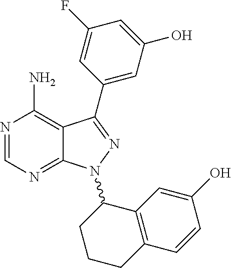 [SMILES: Nc1ncnc2c1c(-c1cc(O)cc(F)c1)nn2C1CCCc2ccc(O)cc21] | 2.161 2.161a, 2.161b |
| 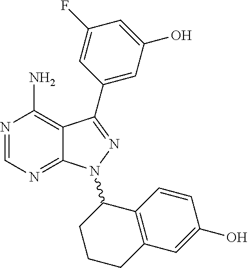 [SMILES: Nc1ncnc2c1c(-c1cc(O)cc(F)c1)nn2C1CCCc2cc(O)ccc21] | 2.162 2.162a, 2.162b |
| 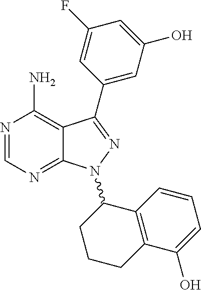 [SMILES: Nc1ncnc2c1c(-c1cc(O)cc(F)c1)nn2C1CCCc2c(O)cccc21] | 2.163 2.163a, 2.163b |
| 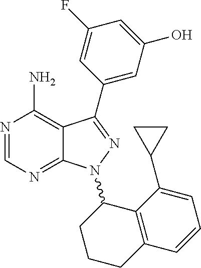 [SMILES: Nc1ncnc2c1c(-c1cc(O)cc(F)c1)nn2C1CCCc2cccc(C3CC3)c21] | 2.164 2.164a, 2.164b |
| 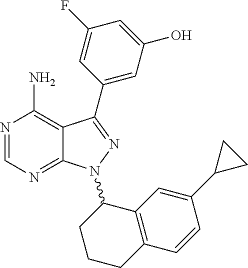 [SMILES: Nc1ncnc2c1c(-c1cc(O)cc(F)c1)nn2C1CCCc2ccc(C3CC3)cc21] | 2.165 2.165a, 2.165b |
| 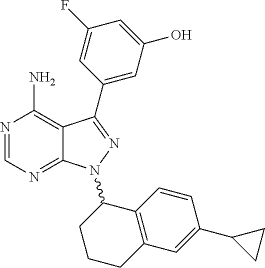 [SMILES: Nc1ncnc2c1c(-c1cc(O)cc(F)c1)nn2C1CCCc2cc(C3CC3)ccc21] | 2.166 2.166a, 2.166b |
| 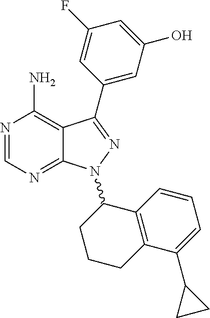 [SMILES: Nc1ncnc2c1c(-c1cc(O)cc(F)c1)nn2C1CCCc2c(C3CC3)cccc21] | 2.167 2.167a, 2.167b |
| 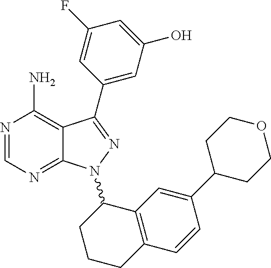 [SMILES: Nc1ncnc2c1c(-c1cc(O)cc(F)c1)nn2C1CCCc2ccc(C3CCOCC3)cc21] | 2.168 2.168a, 2.168b |
| 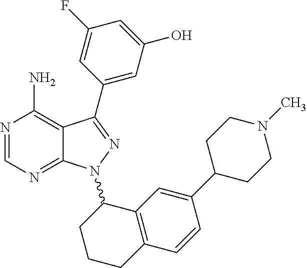 [SMILES: CN1CCC(c2ccc3c(c2)C(n2nc(-c4cc(O)cc(F)c4)c4c(N)ncnc42)CCC3)CC1] | 2.169 2.169a, 2.169b |
| 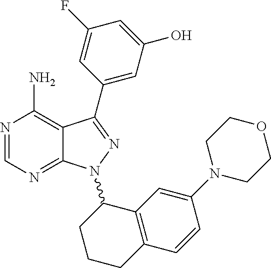 [SMILES: Nc1ncnc2c1c(-c1cc(O)cc(F)c1)nn2C1CCCc2ccc(N3CCOCC3)cc21] | 2.170 2.170a, 2.170b |
| 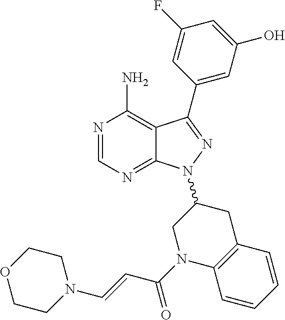 [SMILES: Nc1ncnc2c1c(-c1cc(O)cc(F)c1)nn2C1Cc2ccccc2N(C(=O)/C=C/N2CCOCC2)C1] | 2.171 2.171a, 2.171b |
| 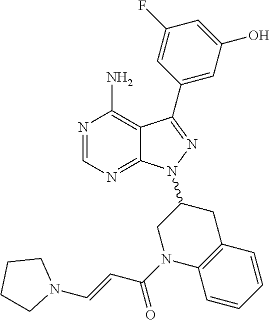 [SMILES: Nc1ncnc2c1c(-c1cc(O)cc(F)c1)nn2C1Cc2ccccc2N(C(=O)/C=C/N2CCCC2)C1] | 2.172 2.172a, 2.172b |
| 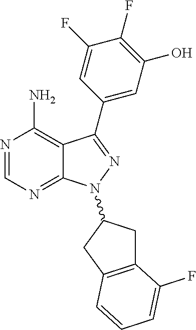 [SMILES: Nc1ncnc2c1c(-c1cc(O)c(F)c(F)c1)nn2C1Cc2cccc(F)c2C1] | 2.173 2.173a, 2.173b |
| 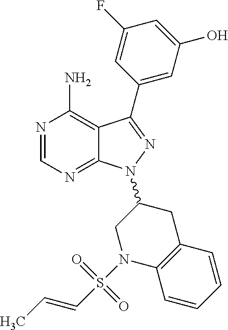 [SMILES: C/C=C/S(=O)(=O)N1CC(n2nc(-c3cc(O)cc(F)c3)c3c(N)ncnc32)Cc2ccccc21] | 2.174 2.174a, 2.174b |
| 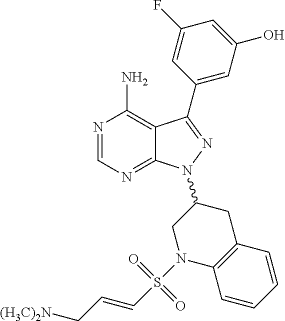 | 2.175 2.175a, 2.175b |
| 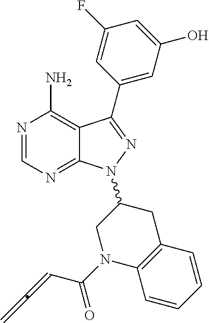 [SMILES: C=C=CC(=O)N1CC(n2nc(-c3cc(O)cc(F)c3)c3c(N)ncnc32)Cc2ccccc21] | 2.176 2.176a, 2.176b |
| 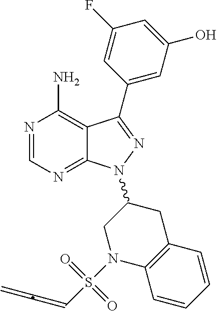 [SMILES: C=C=CS(=O)(=O)N1CC(n2nc(-c3cc(O)cc(F)c3)c3c(N)ncnc32)Cc2ccccc21] | 2.177 2.177a, 2.177b |
| 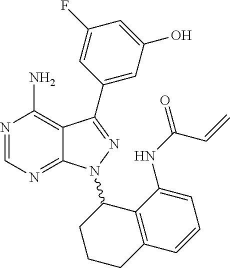 [SMILES: C=CC(=O)Nc1cccc2c1C(n1nc(-c3cc(O)cc(F)c3)c3c(N)ncnc31)CCC2] | 2.178 2.178a, 2.178b |
| 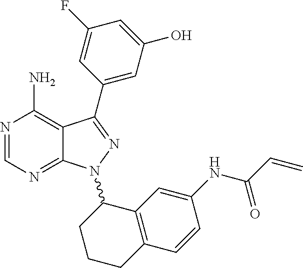 [SMILES: C=CC(=O)Nc1ccc2c(c1)C(n1nc(-c3cc(O)cc(F)c3)c3c(N)ncnc31)CCC2] | 2.179 2.179a, 2.179b |
| 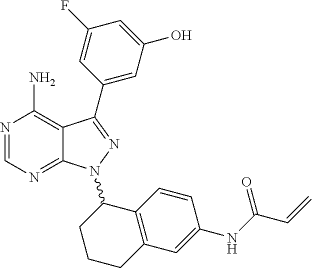 [SMILES: C=CC(=O)Nc1ccc2c(c1)CCCC2n1nc(-c2cc(O)cc(F)c2)c2c(N)ncnc21] | 2.180 2.180a, 2.181b |
| 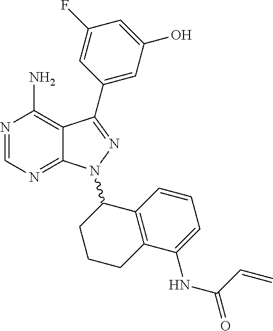 [SMILES: C=CC(=O)Nc1cccc2c1CCCC2n1nc(-c2cc(O)cc(F)c2)c2c(N)ncnc21] | 2.181 2.181a, 2.181b |
| 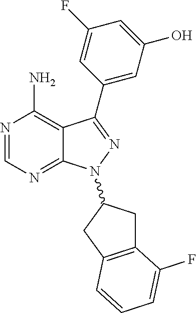 [SMILES: Nc1ncnc2c1c(-c1cc(O)cc(F)c1)nn2C1Cc2cccc(F)c2C1] | 2.182 2.182a, 2.182b |
| 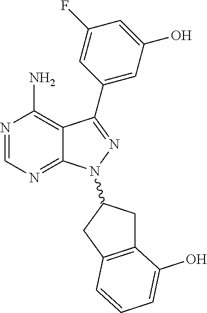 [SMILES: Nc1ncnc2c1c(-c1cc(O)cc(F)c1)nn2C1Cc2cccc(O)c2C1] | 2.183 2.183a, 2.183b |
| 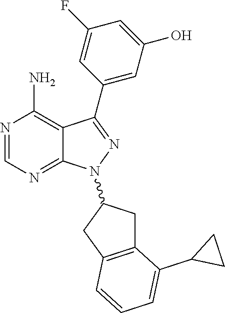 [SMILES: Nc1ncnc2c1c(-c1cc(O)cc(F)c1)nn2C1Cc2cccc(C3CC3)c2C1] | 2.184 2.184a, 2.184b |
| 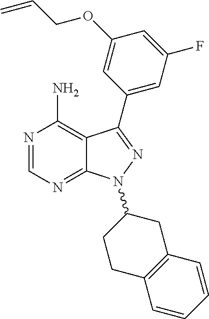 [SMILES: C=CCOc1cc(F)cc(-c2nn(C3CCc4ccccc4C3)c3ncnc(N)c23)c1] | 2.185 2.185a, 2.185b |
| 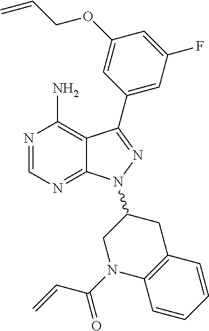 [SMILES: C=CCOc1cc(F)cc(-c2nn(C3Cc4ccccc4N(C(=O)C=C)C3)c3ncnc(N)c23)c1] | 2.186 2.186a, 2.186b |
| 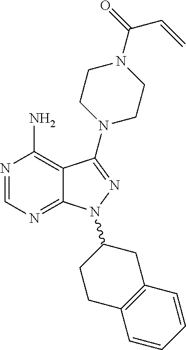 [SMILES: C=CC(=O)N1CCN(c2nn(C3CCc4ccccc4C3)c3ncnc(N)c23)CC1] | 2.187 2.187a, 2.187b |
| 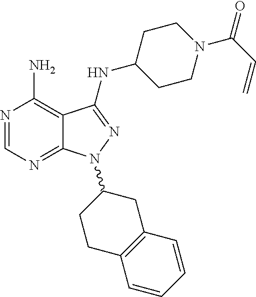 [SMILES: C=CC(=O)N1CCC(Nc2nn(C3CCc4ccccc4C3)c3ncnc(N)c23)CC1] | 2.188 2.188a, 2.188b |
| 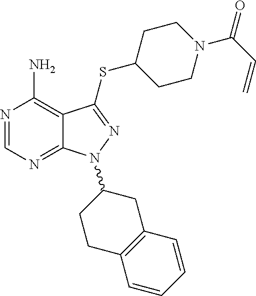 [SMILES: C=CC(=O)N1CCC(Sc2nn(C3CCc4ccccc4C3)c3ncnc(N)c23)CC1] | 2.189 2.189a, 2.189b |
| 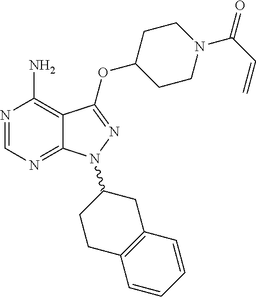 [SMILES: C=CC(=O)N1CCC(Oc2nn(C3CCc4ccccc4C3)c3ncnc(N)c23)CC1] | 2.190 2.190a, 2.190b |
| 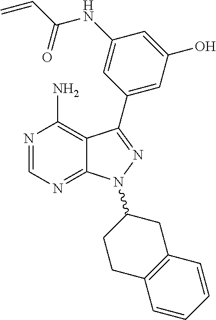 [SMILES: C=CC(=O)Nc1cc(O)cc(-c2nn(C3CCc4ccccc4C3)c3ncnc(N)c23)c1] | 2.191 2.191a, 2.191b |
| 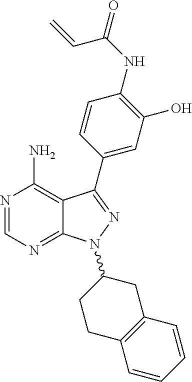 [SMILES: C=CC(=O)Nc1ccc(-c2nn(C3CCc4ccccc4C3)c3ncnc(N)c23)cc1O] | 2.192 2.192a, 2.192b |
| 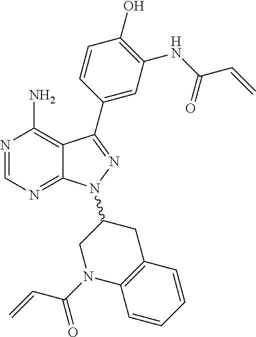 [SMILES: C=CC(=O)Nc1cc(-c2nn(C3Cc4ccccc4N(C(=O)C=C)C3)c3ncnc(N)c23)ccc1O] | 2.193 2.193a, 2.193b |
| 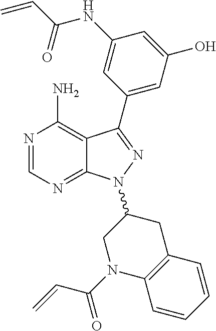 [SMILES: C=CC(=O)Nc1cc(O)cc(-c2nn(C3Cc4ccccc4N(C(=O)C=C)C3)c3ncnc(N)c23)c1] | 2.194 2.194a, 2.194b |
| 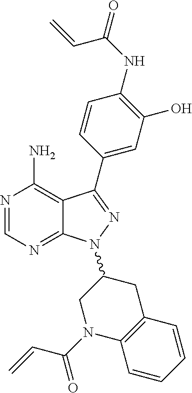 [SMILES: C=CC(=O)Nc1ccc(-c2nn(C3Cc4ccccc4N(C(=O)C=C)C3)c3ncnc(N)c23)cc1O] | 2.195 2.195a, 2.195b |
| 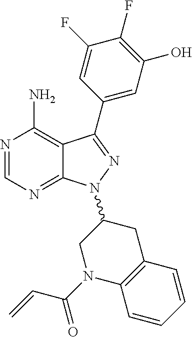 [SMILES: C=CC(=O)N1CC(n2nc(-c3cc(O)c(F)c(F)c3)c3c(N)ncnc32)Cc2ccccc21] | 2.196 2.196a, 2.196b |
| 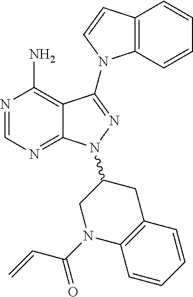 [SMILES: C=CC(=O)N1CC(n2nc(-n3ccc4ccccc43)c3c(N)ncnc32)Cc2ccccc21] | 2.197 2.197a, 2.197b |
| 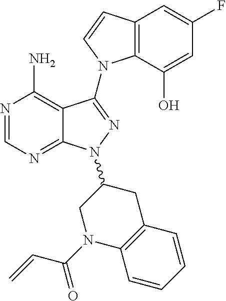 [SMILES: C=CC(=O)N1CC(n2nc(-n3ccc4cc(F)cc(O)c43)c3c(N)ncnc32)Cc2ccccc21] | 2.198 2.198a, 2.198b |
| 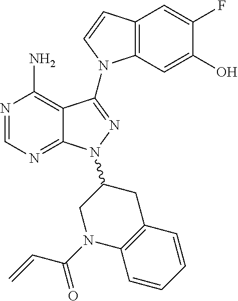 [SMILES: C=CC(=O)N1CC(n2nc(-n3ccc4cc(F)c(O)cc43)c3c(N)ncnc32)Cc2ccccc21] | 2.199 2.199a, 2.199b |
| 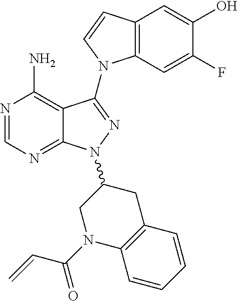 [SMILES: C=CC(=O)N1CC(n2nc(-n3ccc4cc(O)c(F)cc43)c3c(N)ncnc32)Cc2ccccc21] | 2.200 2.200a, 2.200b |
| 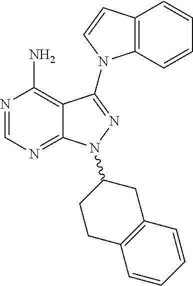 [SMILES: Nc1ncnc2c1c(-n1ccc3ccccc31)nn2C1CCc2ccccc2C1] | 2.201 2.201a, 2.201b |
| 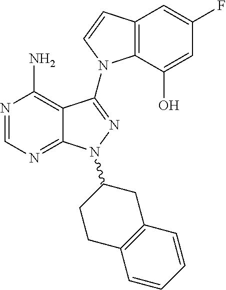 [SMILES: Nc1ncnc2c1c(-n1ccc3cc(F)cc(O)c31)nn2C1CCc2ccccc2C1] | 2.202 2.202a, 2.202b |
| 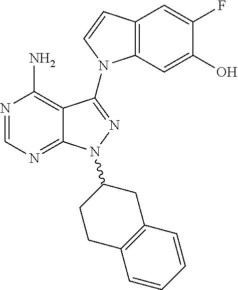 [SMILES: Nc1ncnc2c1c(-n1ccc3cc(F)c(O)cc31)nn2C1CCc2ccccc2C1] | 2.203 2.203a, 2.203b |
| 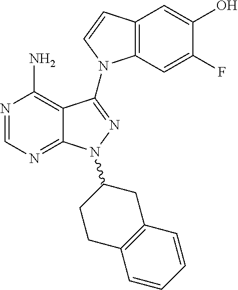 [SMILES: Nc1ncnc2c1c(-n1ccc3cc(O)c(F)cc31)nn2C1CCc2ccccc2C1] | 2.204 2.204a, 2.204b |
| 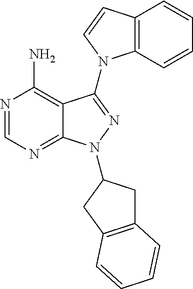 [SMILES: Nc1ncnc2c1c(-n1ccc3ccccc31)nn2C1Cc2ccccc2C1] | 2.205 |
| 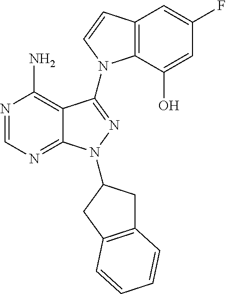 [SMILES: Nc1ncnc2c1c(-n1ccc3cc(F)cc(O)c31)nn2C1Cc2ccccc2C1] | 2.206 |
| 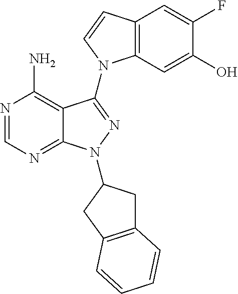 [SMILES: Nc1ncnc2c1c(-n1ccc3cc(F)c(O)cc31)nn2C1Cc2ccccc2C1] | 2.207 |
| 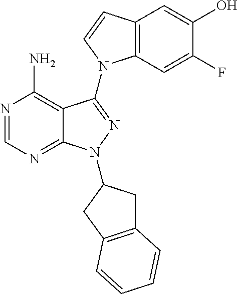 [SMILES: Nc1ncnc2c1c(-n1ccc3cc(O)c(F)cc31)nn2C1Cc2ccccc2C1] | 2.208 |
| 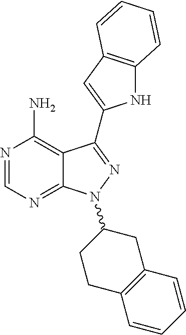 [SMILES: Nc1ncnc2c1c(-c1cc3ccccc3[nH]1)nn2C1CCc2ccccc2C1] | 2.209 2.209a, 2.209b |
| 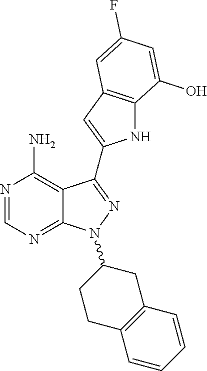 [SMILES: Nc1ncnc2c1c(-c1cc3cc(F)cc(O)c3[nH]1)nn2C1CCc2ccccc2C1] | 2.210 2.210a, 2.210b |
| 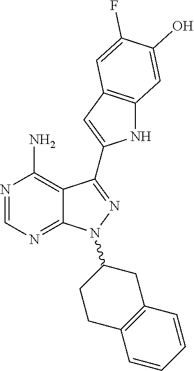 [SMILES: Nc1ncnc2c1c(-c1cc3cc(F)c(O)cc3[nH]1)nn2C1CCc2ccccc2C1] | 2.211 2.211a, 2.211b |
| 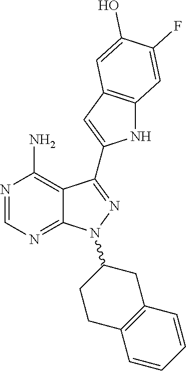 [SMILES: Nc1ncnc2c1c(-c1cc3cc(O)c(F)cc3[nH]1)nn2C1CCc2ccccc2C1] | 2.212 2.212a, 2.212b |
| 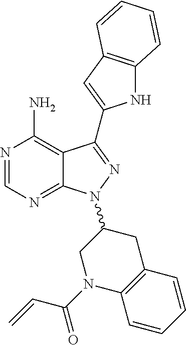 [SMILES: C=CC(=O)N1CC(n2nc(-c3cc4ccccc4[nH]3)c3c(N)ncnc32)Cc2ccccc21] | 2.213 2.213a, 2.213b |
| 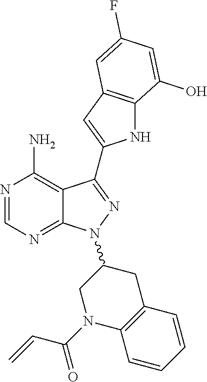 [SMILES: C=CC(=O)N1CC(n2nc(-c3cc4cc(F)cc(O)c4[nH]3)c3c(N)ncnc32)Cc2ccccc21] | 2.214 2.214a, 2.214b |
| 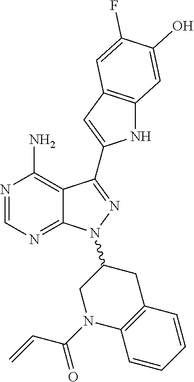 [SMILES: C=CC(=O)N1CC(n2nc(-c3cc4cc(F)c(O)cc4[nH]3)c3c(N)ncnc32)Cc2ccccc21] | 2.215 2.215a, 2.215b |
| 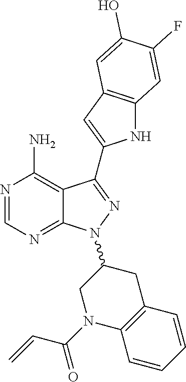 [SMILES: C=CC(=O)N1CC(n2nc(-c3cc4cc(O)c(F)cc4[nH]3)c3c(N)ncnc32)Cc2ccccc21] | 2.216 2.216a, 2.216b |
| 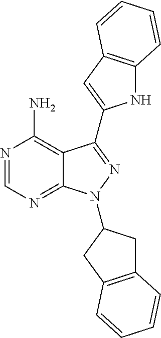 [SMILES: Nc1ncnc2c1c(-c1cc3ccccc3[nH]1)nn2C1Cc2ccccc2C1] | 2.217 |
| 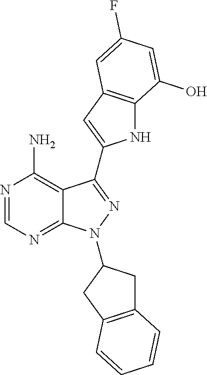 [SMILES: Nc1ncnc2c1c(-c1cc3cc(F)cc(O)c3[nH]1)nn2C1Cc2ccccc2C1] | 2.218 |
| 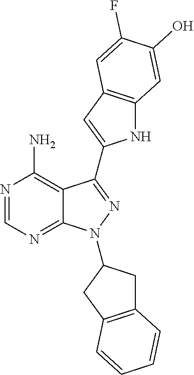 [SMILES: Nc1ncnc2c1c(-c1cc3cc(F)c(O)cc3[nH]1)nn2C1Cc2ccccc2C1] | 2.219 |
| 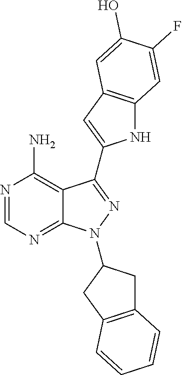 [SMILES: Nc1ncnc2c1c(-c1cc3cc(O)c(F)cc3[nH]1)nn2C1Cc2ccccc2C1] | 2.220 |
| 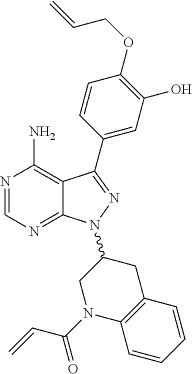 [SMILES: C=CCOc1ccc(-c2nn(C3Cc4ccccc4N(C(=O)C=C)C3)c3ncnc(N)c23)cc1O] | 2.221 2.221a, 2.221b |
| 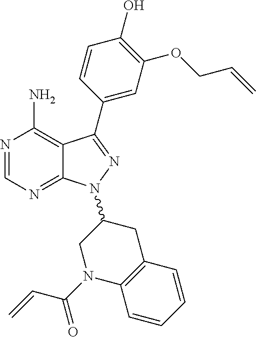 [SMILES: C=CCOc1cc(-c2nn(C3Cc4ccccc4N(C(=O)C=C)C3)c3ncnc(N)c23)ccc1O] | 2.222 2.222a, 2.222b |
| 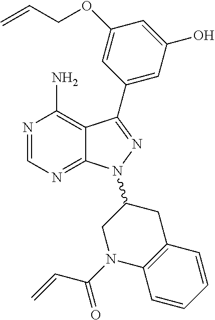 [SMILES: C=CCOc1cc(O)cc(-c2nn(C3Cc4ccccc4N(C(=O)C=C)C3)c3ncnc(N)c23)c1] | 2.223 2.223a, 2.223b |
| 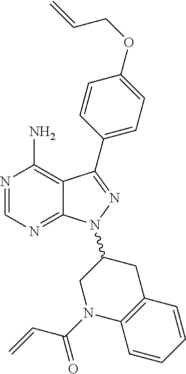 [SMILES: C=CCOc1ccc(-c2nn(C3Cc4ccccc4N(C(=O)C=C)C3)c3ncnc(N)c23)cc1] | 2.224 2.224a, 2.224b |
| 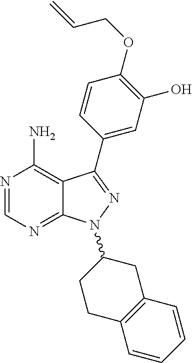 [SMILES: C=CCOc1ccc(-c2nn(C3CCc4ccccc4C3)c3ncnc(N)c23)cc1O] | 2.225 2.225a, 2.225b |
| 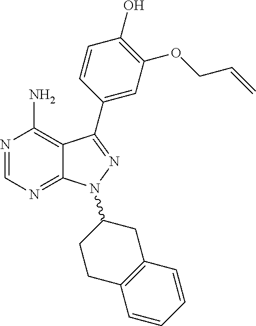 [SMILES: C=CCOc1cc(-c2nn(C3CCc4ccccc4C3)c3ncnc(N)c23)ccc1O] | 2.226 2.226a, 2.226b |
| 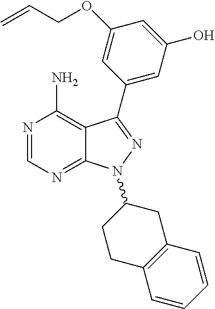 [SMILES: C=CCOc1cc(O)cc(-c2nn(C3CCc4ccccc4C3)c3ncnc(N)c23)c1] | 2.227 2.227a, 2.227b |
| 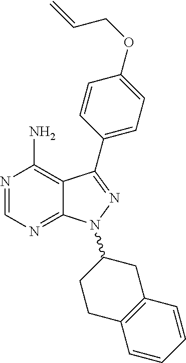 [SMILES: C=CCOc1ccc(-c2nn(C3CCc4ccccc4C3)c3ncnc(N)c23)cc1] | 2.228 2.228a, 2.228b |
| 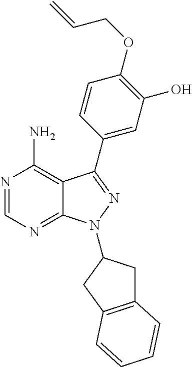 [SMILES: C=CCOc1ccc(-c2nn(C3Cc4ccccc4C3)c3ncnc(N)c23)cc1O] | 2.229 |
| 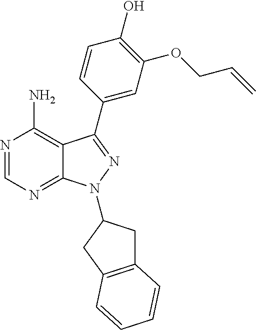 [SMILES: C=CCOc1cc(-c2nn(C3Cc4ccccc4C3)c3ncnc(N)c23)ccc1O] | 2.230 |
| 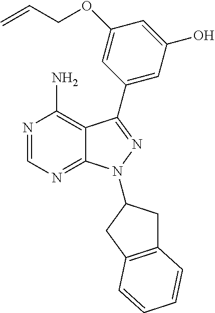 [SMILES: C=CCOc1cc(O)cc(-c2nn(C3Cc4ccccc4C3)c3ncnc(N)c23)c1] | 2.231 |
| 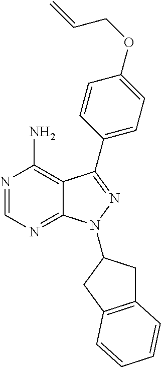 [SMILES: C=CCOc1ccc(-c2nn(C3Cc4ccccc4C3)c3ncnc(N)c23)cc1] | 2.232 |
| 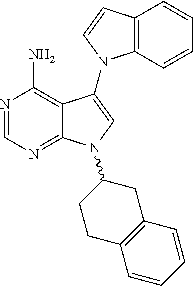 [SMILES: Nc1ncnc2c1c(-n1ccc3ccccc31)cn2C1CCc2ccccc2C1] | 2.233 2.233a, 2.233b |
| 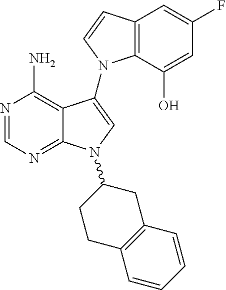 [SMILES: Nc1ncnc2c1c(-n1ccc3cc(F)cc(O)c31)cn2C1CCc2ccccc2C1] | 2.234 2.234a, 2.234b |
| 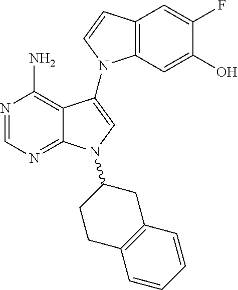 [SMILES: Nc1ncnc2c1c(-n1ccc3cc(F)c(O)cc31)cn2C1CCc2ccccc2C1] | 2.235 2.235a, 2.235b |
| 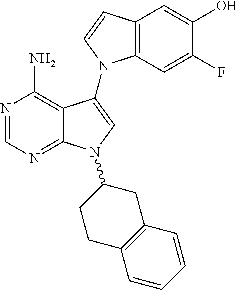 [SMILES: Nc1ncnc2c1c(-n1ccc3cc(O)c(F)cc31)cn2C1CCc2ccccc2C1] | 2.236 2.236a, 2.236b |
| 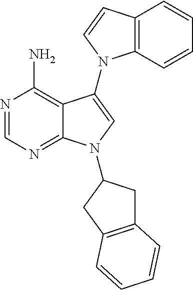 [SMILES: Nc1ncnc2c1c(-n1ccc3ccccc31)cn2C1Cc2ccccc2C1] | 2.237 |
| 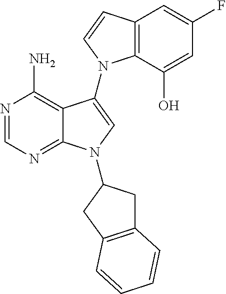 [SMILES: Nc1ncnc2c1c(-n1ccc3cc(F)cc(O)c31)cn2C1Cc2ccccc2C1] | 2.238 |
| 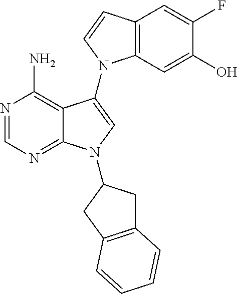 [SMILES: Nc1ncnc2c1c(-n1ccc3cc(F)c(O)cc31)cn2C1Cc2ccccc2C1] | 2.239 |
| 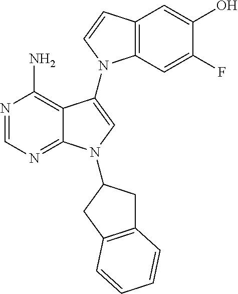 [SMILES: Nc1ncnc2c1c(-n1ccc3cc(O)c(F)cc31)cn2C1Cc2ccccc2C1] | 2.240 |
| 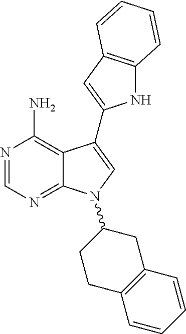 [SMILES: Nc1ncnc2c1c(-c1cc3ccccc3[nH]1)cn2C1CCc2ccccc2C1] | 2.241 2.241a, 2.241b |
| 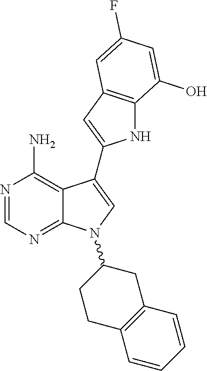 [SMILES: Nc1ncnc2c1c(-c1cc3cc(F)cc(O)c3[nH]1)cn2C1CCc2ccccc2C1] | 2.242 2.242a, 2.242b |
| 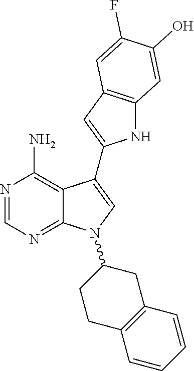 [SMILES: Nc1ncnc2c1c(-c1cc3cc(F)c(O)cc3[nH]1)cn2C1CCc2ccccc2C1] | 2.243 2.243a, 2.243b |
| 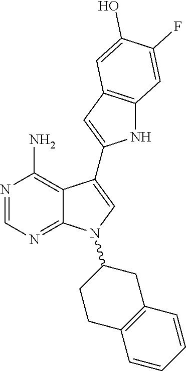 [SMILES: Nc1ncnc2c1c(-c1cc3cc(O)c(F)cc3[nH]1)cn2C1CCc2ccccc2C1] | 2.244 2.244a, 2.244b |
| 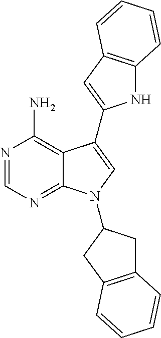 [SMILES: Nc1ncnc2c1c(-c1cc3ccccc3[nH]1)cn2C1Cc2ccccc2C1] | 2.245 |
| 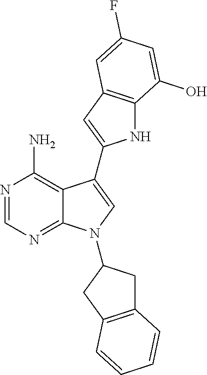 [SMILES: Nc1ncnc2c1c(-c1cc3cc(F)cc(O)c3[nH]1)cn2C1Cc2ccccc2C1] | 2.246 |
| 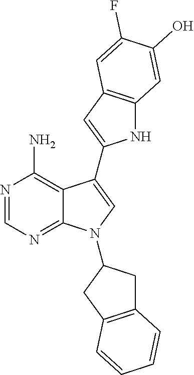 [SMILES: Nc1ncnc2c1c(-c1cc3cc(F)c(O)cc3[nH]1)cn2C1Cc2ccccc2C1] | 2.47 |
| 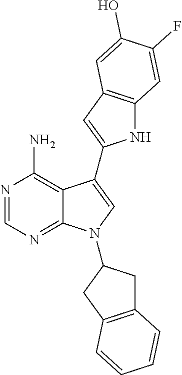 [SMILES: Nc1ncnc2c1c(-c1cc3cc(O)c(F)cc3[nH]1)cn2C1Cc2ccccc2C1] | 2.248 |
| 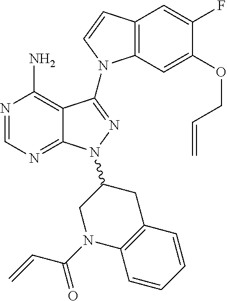 [SMILES: C=CCOc1cc2c(ccn2-c2nn(C3Cc4ccccc4N(C(=O)C=C)C3)c3ncnc(N)c23)cc1F] | 2.249 2.249a, 2.249b |
| 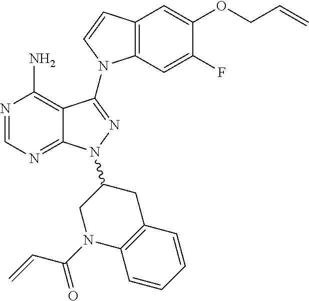 [SMILES: C=CCOc1cc2ccn(-c3nn(C4Cc5ccccc5N(C(=O)C=C)C4)c4ncnc(N)c34)c2cc1F] | 2.250 2.250a, 2.250b |
| 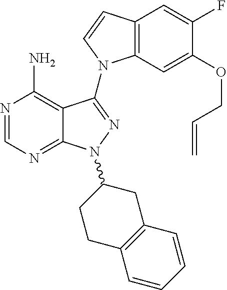 [SMILES: C=CCOc1cc2c(ccn2-c2nn(C3CCc4ccccc4C3)c3ncnc(N)c23)cc1F] | 2.251 2.251a, 2.251b |
| 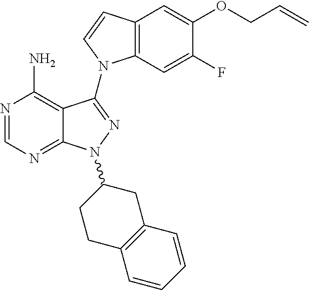 [SMILES: C=CCOc1cc2ccn(-c3nn(C4CCc5ccccc5C4)c4ncnc(N)c34)c2cc1F] | 2.252 2.252a, 2.252b |
| 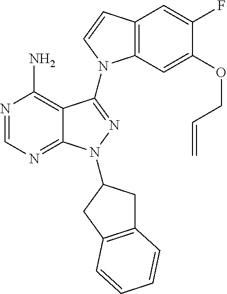 [SMILES: C=CCOc1cc2c(ccn2-c2nn(C3Cc4ccccc4C3)c3ncnc(N)c23)cc1F] | 2.253 |
| 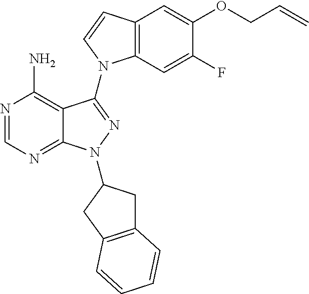 [SMILES: C=CCOc1cc2ccn(-c3nn(C4Cc5ccccc5C4)c4ncnc(N)c34)c2cc1F] | 2.254 |
| 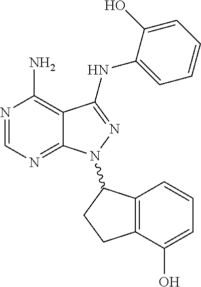 [SMILES: Nc1ncnc2c1c(Nc1ccccc1O)nn2C1CCc2c(O)cccc21] | 2.255 2.255a, 2.255b |
| 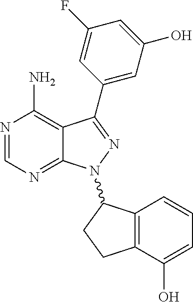 [SMILES: Nc1ncnc2c1c(-c1cc(O)cc(F)c1)nn2C1CCc2c(O)cccc21] | 2.256 2.256a, 2.256b |
| 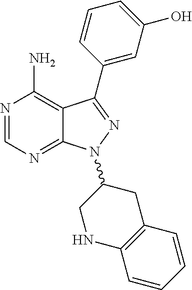 [SMILES: Nc1ncnc2c1c(-c1cccc(O)c1)nn2C1CNc2ccccc2C1] | 2.257 2.257a, 2.257b |
| 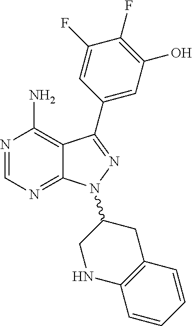 [SMILES: Nc1ncnc2c1c(-c1cc(O)c(F)c(F)c1)nn2C1CNc2ccccc2C1] | 2.258 2.258a, 2.258b |
| 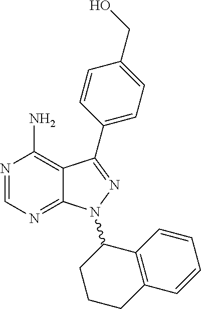 [SMILES: Nc1ncnc2c1c(-c1ccc(CO)cc1)nn2C1CCCc2ccccc21] | 2.259 2.259a, 2.259b |
| 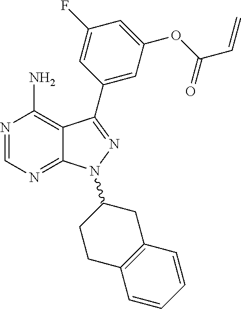 [SMILES: C=CC(=O)Oc1cc(F)cc(-c2nn(C3CCc4ccccc4C3)c3ncnc(N)c23)c1] | 2.260 2.260a, 2.260b |
| 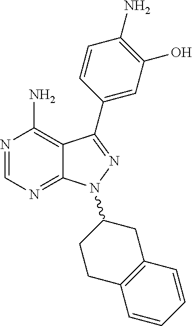 [SMILES: Nc1ccc(-c2nn(C3CCc4ccccc4C3)c3ncnc(N)c23)cc1O] | 2.261 2.261a, 2.261b |
| 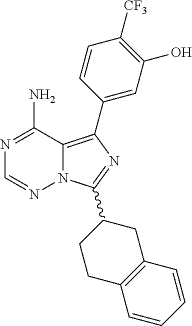 [SMILES: Nc1ncnn2c(C3CCc4ccccc4C3)nc(-c3ccc(C(F)(F)F)c(O)c3)c12] | 2.262 2.262a, 2.262b |
| 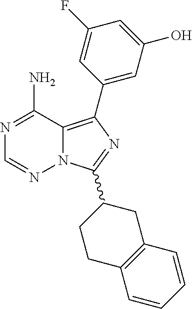 [SMILES: Nc1ncnn2c(C3CCc4ccccc4C3)nc(-c3cc(O)cc(F)c3)c12] | 2.263 2.263a, 2.263b |
| 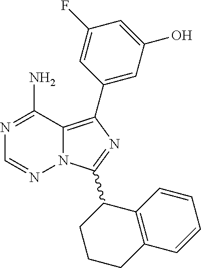 [SMILES: Nc1ncnn2c(C3CCCc4ccccc43)nc(-c3cc(O)cc(F)c3)c12] | 2.264 2.264a, 2.264b |
| 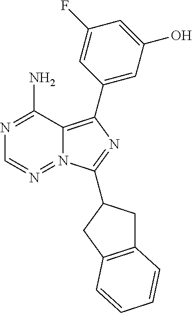 | 2.265 |
| 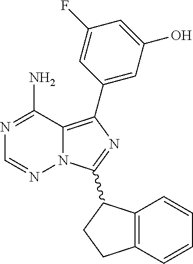 [SMILES: Nc1ncnn2c(C3CCc4ccccc43)nc(-c3cc(O)cc(F)c3)c12] | 2.266 2.266a, 2.266b |
| 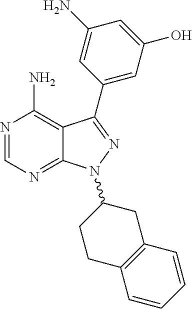 [SMILES: Nc1cc(O)cc(-c2nn(C3CCc4ccccc4C3)c3ncnc(N)c23)c1] | 2.267 2.267a, 2.267b |
| 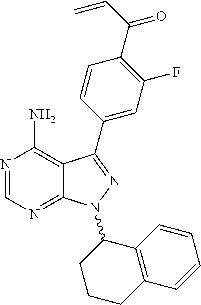 [SMILES: C=CC(=O)c1ccc(-c2nn(C3CCCc4ccccc43)c3ncnc(N)c23)cc1F] | 2.268 2.268a, 2.268b |
| 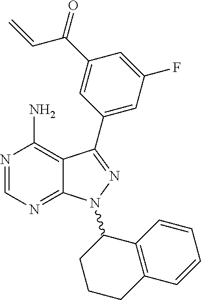 [SMILES: C=CC(=O)c1cc(F)cc(-c2nn(C3CCCc4ccccc43)c3ncnc(N)c23)c1] | 2.269 2.269a, 2.269b |
| 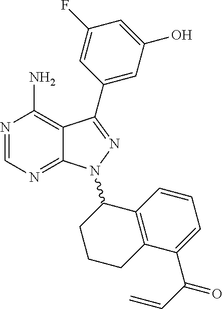 [SMILES: C=CC(=O)c1cccc2c1CCCC2n1nc(-c2cc(O)cc(F)c2)c2c(N)ncnc21] | 2.270 2.270a, 2.270b |
| 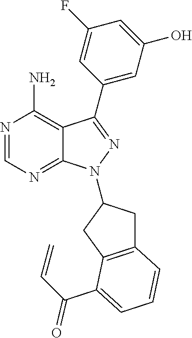 [SMILES: C=CC(=O)c1cccc2c1CC(n1nc(-c3cc(O)cc(F)c3)c3c(N)ncnc31)C2] | 2.271 |
| 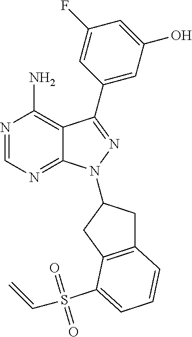 [SMILES: C=CS(=O)(=O)c1cccc2c1CC(n1nc(-c3cc(O)cc(F)c3)c3c(N)ncnc31)C2] | 2.272 |
| 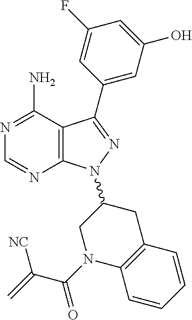 [SMILES: C=C(C#N)C(=O)N1CC(n2nc(-c3cc(O)cc(F)c3)c3c(N)ncnc32)Cc2ccccc21] | 2.273 2.273a, 2.273b |
| 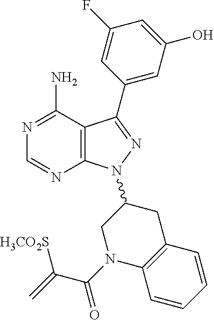 | 2.274 2.274a, 2.274b |
| 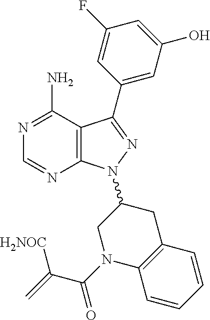 [SMILES: C=C(C(N)=O)C(=O)N1CC(n2nc(-c3cc(O)cc(F)c3)c3c(N)ncnc32)Cc2ccccc21] | 2.275 2.275a, 2.275b |
| 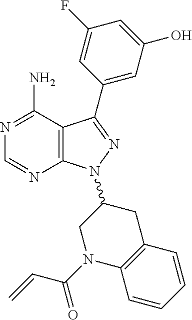 [SMILES: C=CC(=O)N1CC(n2nc(-c3cc(O)cc(F)c3)c3c(N)ncnc32)Cc2ccccc21] | 2.276 2.276a, 2.276b |
| 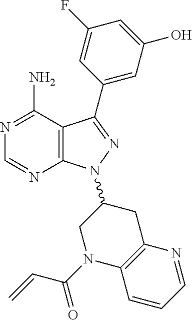 [SMILES: C=CC(=O)N1CC(n2nc(-c3cc(O)cc(F)c3)c3c(N)ncnc32)Cc2ncccc21] | 2.277 2.277a, 2.277b |
| 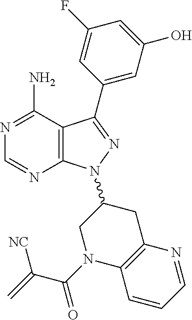 [SMILES: C=C(C#N)C(=O)N1CC(n2nc(-c3cc(O)cc(F)c3)c3c(N)ncnc32)Cc2ncccc21] | 2.278 2.278a, 2.278b |

Representative examples of compounds detailed herein, including intermediates and final compounds according to the invention are depicted in the tables below. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the invention embraces all salts and solvates of the compounds depicted here, as well as the non-salt and non-solvate form of the compound, as is well understood by the skilled artisan. In some embodiments, the salts of the compounds of the invention are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted.

The invention also includes any or all of the stereochemical forms, including any enantiomeric or diastereomeric forms of the compounds described. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

Pharmaceutical compositions of any of the compounds detailed herein are embraced by this invention. Thus, the invention includes pharmaceutical compositions comprising a compound of the invention or a pharmaceutically acceptable salt thereof and a pharmaceutically acceptable carrier or excipient. In one aspect, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. Taking compound 1 as an example, a composition of substantially pure compound 1 intends a composition that contains no more than 35% impurity, wherein the impurity denotes a compound other than compound 1 or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other embodiments, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form. For instance, a composition of substantially pure (S) compound means that the composition contains no more than 15% or no more than 10% or no more than 5% or no more than 3% or no more than 1% of the (R) form of the compound.

In one variation, the compounds herein are synthetic compounds prepared for administration to an individual. In another variation, compositions are provided containing a compound in substantially pure form. In another variation, the invention embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

### General Description of Biological Assays

The binding properties of compounds disclosed herein to a panel of kinases, including BTK and PI3Kδ may be determined. Binding properties may be assessed by methods known in the art, such as competitive binding assays, or kinase assays. In one variation, compounds are assessed by the kinase assays detailed herein. Other assays are known to those skilled in the art, such as, for example, those presented and discussed in US2013/0165395A1 (for BTK), and US2012/0053166A1 (for PIKδ). Compounds disclosed herein may also be tested in cell-based assays or in in vivo models for further characterization.

### Therapeutic Uses and Methods

Compounds and compositions provided herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

While not being bound by theory, it is believed that the effects of the compounds presented herein may be attributed to the ability of the compounds to inhibit both BTK kinase and PI3Kδ kinase, the result being a reduction or elimination of signaling in the BCR pathway, and reduction in downstream events of that pathway. Compounds that negatively regulate both BTK and PI3Kδ expression or activity can be used as dual BTK-PI3Kδ inhibitors in the methods of the invention. In one aspect, the compounds may have a synergistic effect. In another aspect, the compounds may have an additive effect. In another aspect, the compounds may inhibit just one of the BTK and PI3Kδ kinases. In another aspect, the compounds may inhibit one of the BTK and PI3Kδ kinases and also block the resistance mechanism of the remaining kinase. In another aspect, the compounds may block the resistance mechanisms of both BTK and PI3Kδ kinases.

The compounds of the invention are inhibitors of kinase activity, in particular BTK and PI3Kδ activity. Compounds which are BTK and/or PI3Kδ inhibitors may be used in the treatment of disorders wherein the underlying pathology is (at least in part) attributable to inappropriate BTK and/or PI3Kδ activity. “Inappropriate BTK and/or PI3Kδ activity” refers to any kinase activity that deviates from normal BTK and/or PI3Kδ activity expected in a particular patient, and which may take the form of, for instance, an abnormal increase in activity, or an aberration in the timing and/or control of BTK and/or PI3Kδ activity. Such inappropriate activity may result then, for example, from overexpression or mutation of the protein kinase(s) leading to inappropriate or uncontrolled activation. Accordingly, in another aspect, the invention is directed to methods of treating such disorders. In some aspects, provided herein are methods for treating diseases impacted by a resistance to BTK inhibition. In some aspects, provided herein are methods for treating diseases impacted by a resistance to PI3Kδ inhibition.

In some aspects, provided herein is a method for utilizing compounds of the invention as a monotherapy to treat both BTK-mediated disorders and PI3Kδ-mediated disorders. In some aspects, the monotherapy can overcome resistance to treatment of either a BTK-mediated disorder alone, or a PI3Kδ-mediated disorder alone. In some aspects, the monotherapy can negate the need for combination treatment with a second therapeutic agent previously required to overcome resistance to treatment. In some aspects, provided is a method to treat a subject that is resistant or has developed a resistance to therapeutic agents.

In some aspects, presented herein is a method for suppressing a function of basophils and/or mast cells, and thereby enabling treatment of diseases or disorders characterized by excessive or undesirable basophil and/or mast cell activity. A compound of the invention can be used that inhibits the expression or activity of both BTK and PI3Kδ in the basophils and/or mast cells.

In some aspects, provided herein is a method for suppressing osteoclastogenesis. A compound of the invention can be used that delays the onset and/or development of bone loss arising from osteoclast-associated bone disorders, such as for example bone metastasis, osteoarthritis and rheumatoid arthritis.

Compounds provided herein may be used in a method of delaying the onset and/or development of a disease or condition associated with excessive or undesirable basophil and/or mast cell activity, or with basophil and/or mast cell dysfunction. The compounds provided herein may be used in a method of delaying the onset of a disease or condition that is responsive to a decrease in basophil and/or mast cell activity. The compounds as provided herein may also be used in a method of delaying the onset and/or development of any indications presented below.

In some aspects, compounds presented herein selectively inhibit PI3Kδ over related PI3K isoforms. The advantage of a PI3Kδ selective inhibitor which targets cells mediating inflammation and cancer cells, wherein potential clinical indications include cancer, rheumatoid arthritis, asthma, allergies and COPD, is that treatment is well tolerated, and side effects like hyperinsulinemia are avoided. In some aspects, compounds of the invention provide therapeutic benefits to treating hematologic malignancies without adversely affecting insulin signaling.

In some aspects, presented herein are methods of selectively inhibiting PIKδ. In other aspects, presented herein are methods of inhibiting PI3Kβ and/or PI3Kγ.

In each of the above embodiments, aspects and variations, the application also provides compounds of the formulae (I), (II) or (III), or any one of a compound of the formula A-1 to A-10, A-1a to A-10e, A-1a-1 to A-10e-8, (B-1), (B-2), (B-3), or (B-4); or a compound of Table 1 or 2, or a pharmaceutically acceptable salt thereof, for use in a method of treating cancer or for the treatment of an autoimmune disease. In one aspect of the above, the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma.

In some aspects, the compounds presented herein inhibit both Btk kinase and PI3Kδ kinase activity with an in vitro IC₅₀ of less than 10 μM. (e.g., less than 1 μM, less than 0.5 μM, less than 0.4 μM, less than 0.3 μM, less than 0.1 μM, less than 0.08 μM, less than 0.06 μM, less than 0.05 μM, less than 0.04 μM, less than 0.03 μM, less than 0.02 μM, less than 0.01 μM, less than 0.008 μM, less than 0.006 μM, less than 0.005 μM, less than 0.004 μM, less than 0.003 μM, less than 0.002 μM, or less than 0.001 μM. In some aspects, the compounds inhibit both Btk kinase and PI3Kδ kinase activity with an in vitro IC₅₀ of less than 0.1 μM. In some aspects, the compounds inhibit both Btk kinase and PI3Kδ kinase activity with an in vitro IC₅₀ of less than 0.05 μM.

Compounds provided herein, such as the dual BTK and PI3Kδ kinase inhibitors provided herein, are expected to find use in therapy, particularly in disease indications resulting from an inappropriate activation of the BCR pathway, B-cell malignancies, or diseases otherwise benefiting from inhibition of BTK or PI3Kδ activity.

In one aspect, provided herein is a method for treating diseases impacted by one or more of BTK and/or PI3Kδ kinases, in a subject in need thereof by administering to the subject thereof a composition containing a therapeutically effective amount of a compound having a structure presented herein.

In another aspect, the invention includes a method for suppressing a function of basophils and/or mast cells, and thereby enabling treatment of diseases or disorders characterized by excessive or undesirable basophil and/or mast cell activity. According to the method, a compound of the invention can be used that selectively inhibits the expression or activity of BTK or PI3Kδ in the basophils and/or mast cells. Preferably, the method employs an inhibitor in an amount sufficient to inhibit stimulated histamine release by the basophils and/or mast cells.

In some aspects, the subject in need is suffering from an autoimmune disease, a heteroimmune condition, an inflammatory disease, cancer, a thromboembolic disorder, a respiratory disease.

In some aspects, the autoimmune disease includes, but is not limited to, inflammatory bowel disease, arthritis, lupus, inflammatory bowel disease (including Crohn's disease and ulcerative colitis), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease Sjogren's syndrome, multiple sclerosis, Guillain-Barre syndrome, acute disseminated encephalomyelitis, Addison's disease, opsoclonus-myoclonus syndrome, ankylosing spondylitisis, antiphospholipid antibody syndrome, aplastic anemia, autoimmune hepatitis, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behyet's disease, chronic fatigue, dysautonomia, endometriosis, interstitial cystitis, neuromyotonia, scleroderma, vulvodynia, allergic rhinitis, asthma and COPD.

In some aspects, the heteroimmune condition or disease includes, but is not limited to, graft versus host disease, transplantation, transfusion, anaphylaxis, allergy, type I hypersensitivity, allergic conjunctivitis, allergic rhinitis, and atopic dermatitis.

In some aspects, the inflammatory disease includes, but is not limited to, asthma, appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, and vulvitis.

In some aspects, the cancer is associated with abnormal BTK or PI3Kδ activity compared to activity in a subject without cancer. In some aspects, the cancer is a B-cell proliferative disorder, and includes, but is not limited to, diffuse large B cell lymphoma, follicular lymphoma, chronic lymphocytic lymphoma, chronic lymphocytic leukemia (CLL), B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, burkitt lymphoma/leukemia, lymphomatoid granulomatosis, acute lymphocytic leukemia (ALL), acute myeloid leukemia (AML), small lymphocytic lymphoma (SLL), multiple myeloma (MM), non-Hodgkin's lymphoma (NHL), and mantle cell lymphoma (MCL). In some aspects, the cancer is B-cell or T-cell ALL. In some aspects, the cancer is Hodgkin's lymphoma. In some aspects, the cancer is breast, lung, colon, prostate or ovarian cancer. In some aspects, lymphoma is a mature (peripheral) B-cell neoplasm. In specific embodiments, the mature B-cell neoplasm is selected from the group consisting of B-cell chronic lymphocytic leukemia/small lymphocytic lymphoma; B-cell prolymphocytic leukemia; Lymphoplasmacytic lymphoma; Marginal zone lymphoma, such as Splenic marginal zone B-cell lymphoma (+/− villous lymphocytes), Nodal marginal zone lymphoma (+/monocytoid B-cells), and Extranodal marginal zone B-cell lymphoma of mucosa-associated lymphoid tissue (MALT) type; Hairy cell leukemia; Plasma cell myeloma/plasmacytoma; Follicular lymphoma, follicle center; Mantle cell lymphoma; Diffuse large cell B-cell lymphoma (including Mediastinal large B-cell lymphoma, Intravascular large B-cell lymphoma, and Primary effusion lymphoma); and Burkitt's lymphoma/Burkitt's cell leukemia.

In some aspects, the thromboembolic disorder includes, but is not limited to, myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some aspects, the inflammatory disease includes, but is not limited to, asthma, inflammatory bowel disease (including Crohn's disease and ulcerative colitis), appendicitis, blepharitis, bronchiolitis, bronchitis, bursitis, cervicitis, cholangitis, cholecystitis, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, hepatitis, hidradenitis suppurativa, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, proctitis, prostatitis, pyelonephritis, rhinitis, salpingitis, sinusitis, stomatitis, synovitis, tendonitis, tonsillitis, uveitis, vaginitis, vasculitis, or vulvitis.

In some aspects, the respiratory disease is asthma. In some aspects, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child onset asthma, adult-onset asthma, cough-variant asthma, occupational asthma, steroid-resistant asthma, and seasonal asthma.

In some aspects, provided herein are methods for treating the diseases presented above, by administering to a subject in need thereof a composition containing a therapeutically effective amount of a compound that forms a covalent bond with one or both of BTK and/or PIKδ. In some aspects, the compound forms a covalent bound with the activated form of BTK and/or PIKδ. In some aspects, the compound irreversibly inhibits one or both of BTK and/or PI3Kδ to which it is covalently bound. In some aspects, the compound forms a covalent bond with a cysteine residue on one or both of BTK and/or PIK6.

In some aspects, the subject is refractory to chemotherapy treatment, or in relapse after treatment with chemotherapy. In some aspects, the subject is a de novo patient. In some aspects, the method comprises reducing the level of BTK and/or PI3Kδ activity in said patient. In some aspects, the subject is a human subject.

In one aspect, the methods presented herein comprise administering to an individual (e.g., in a human) a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In one aspect are provided methods for treating autoimmune diseases in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In another aspect are provided methods for treating a heteroimmune condition in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In another aspect are provided methods for treating an inflammatory disease in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In another aspect are provided methods for treating cancer in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a therapeutically effective amount of compound provided herein, or a pharmaceutically acceptable salt thereof; a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In some embodiments, the present application provides a method for the treatment of cancer or for the treatment of an autoimmune disease to a patient comprising the administration of a therapeutically effective amount of a Btk inhibitor to a patient in need thereof, wherein the Btk inhibitor is a compound of the formulae (I), (II) or (III), or any one of a compound of the formula A-1 to A-10, A-1a to A-10e, or A-1a-1 to A-10e-8; or a compound of Table 1 or 2, or a pharmaceutically acceptable salt thereof. In some embodiments the method, the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma. In some embodiments of the method, the autoimmune disease is selected from the group consisting of rheumatoid arthritis and systemic lupus erythematosis.

In some embodiments, the present application provides a method for the treatment of cancer or for the treatment of an autoimmune disease to a patient comprising the administration of a therapeutically effective amount of a PI3Kδ inhibitor to a patient in need thereof, wherein the PI3Kδ inhibitor is a compound of the formulae (I), (II) or (III), or any one of a compound of the formula A-1 to A-10, A-1a to A-10e, or A-1a-1 to A-10e-8; or a compound of Table 1 or 2, or a pharmaceutically acceptable salt thereof. In some embodiments of the method, the cancer is selected from the group consisting of chronic lymphocytic leukemia, mantle cell lymphoma, B cell non Hodgkin lymphoma, multiple myeloma and acute myeloid lymphoma. In some embodiments of the method, the autoimmune disease is selected from the group consisting of rheumatoid arthritis, allergic asthma and myocardial infarction.

In some embodiments, the present application provides a method for the treatment of cancer or for the treatment of an autoimmune disease to a patient comprising the administration of a therapeutically effective amount of a dual Btk and PI3Kδ inhibitor to a patient in need thereof, wherein the dual Btk and PI3Kδ inhibitor is a compound of the formulae (I), (II) or (III), or any one of a compound of the formula A-1 to A-10, A-1a to A-10e, or A-1a-1 to A-10e-8; or a compound of Table 1 or 2, or a pharmaceutically acceptable salt thereof. In some embodiments of the method, the cancer is selected from the group consisting of chronic lymphocytic leukemia, mantle cell lymphoma, multiple myeloma and B cell non Hodgkin lymphoma. In some embodiments of the method, the autoimmune disease is rheumatoid arthritis.

Additionally provided is a method for treating a tumor comprising contacting the tumor with an effective amount of one or more compounds provided herein, or a salt thereof. In one aspect of the method, a compound or salt thereof is administered to an individual in need of tumor treatment. In one aspect, the treatment results in a reduction of the tumor size. In another aspect, the treatment slows or prevents tumor growth and/or metastasis.

Any of the methods of treatment provided herein may be used to treat a primary tumor. Any of the methods of treatment provided herein may also be used to treat a metastatic cancer (that is, cancer that has metastasized from the primary tumor). Any of the methods of treatment provided herein may be used to treat cancer at an advanced stage. Any of the methods of treatment provided herein may be used to treat cancer at a locally advanced stage. Any of the methods of treatment provided herein may be used to treat early stage cancer. Any of the methods of treatment provided herein may be used to treat cancer in remission. In some of the embodiments of any of the methods of treatment provided herein, the cancer has reoccurred after remission. In some embodiments of any of the methods of treatment provided herein, the cancer is progressive cancer.

In another aspect are provided methods for treating a thromboembolic disorder in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

In another aspect are provided methods for treating a respiratory disease in an individual (e.g., in a human) comprising administering to the individual an effective amount of a compound described herein or a pharmaceutically acceptable salt thereof or a composition comprising the compound or salt thereof. In one aspect, the methods presented herein comprise administering to the individual a compound provided herein, or a pharmaceutically acceptable salt thereof, a compound according to any one or more of formulae (I), (II), (III), A-1 to A-10, A-1a to A-10e, or A-1a-1 to A-10e-8; or a compound of Table 1 or 2, or an isomer thereof, or a salt (such as a pharmaceutically acceptable salt) of any of the foregoing.

### Dosing and Method of Administration

The dose of a compound administered to an individual (such as a human) may vary with the particular compound or salt thereof, the method of administration, and the particular indication being treated. The amount should be sufficient to produce a desirable response, such as a therapeutic or prophylactic response against the disease. In some embodiments, the amount of the compound or salt thereof is a therapeutically effective amount. In some embodiments, the amount of the compound or salt thereof is a prophylactically effective amount. In some embodiments, the amount of compound or salt thereof is below the level that induces a toxicological effect (e.g., an effect above a clinically acceptable level of toxicity) or is at a level where a potential side effect can be controlled or tolerated when the composition is administered to the individual.

Methods as provided herein may comprise administering to an individual a pharmacological composition that contains an effective amount of a compound and a pharmaceutically acceptable carrier. The effective amount of the compound may in one aspect be a dose of between about 0.01 and about 100 mg/kg.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein.

Articles of manufacture comprising a compound of the invention, or a salt or solvate thereof, in a suitable container are provided. The container may be a vial, jar, ampoule, preloaded syringe, i.v. bag, and the like.

The compound may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein can be used in the preparation of a formulation, such as a pharmaceutical formulation, by combining the compound or compounds as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants.

Any of the compounds described herein can be formulated in a tablet in any dosage form described, for example, a compound as described herein or a pharmaceutically acceptable salt thereof can be formulated as a 10 mg tablet.

The compound may be administered to an individual in accordance with an effective dosing regimen for a desired period of time or duration, such as at least about one month, at least about 2 month, at least about 3 month, at least about 6 month, or at least about 12 months or longer, which in some embodiments may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent (e.g., once daily dosing for 7 days followed by no doses for 7 days, repeated for any 14 day time period, such as about 2 month, about 4 month, about 6 months or more). Any of the dosing frequencies can employ any of the compounds described herein together with any of the dosages described herein.

Compositions comprising a compound provided herein are also described. In one variation, the composition comprises a compound and a pharmaceutically acceptable carrier or excipient. In another variation, a composition of substantially pure compound is provided.

### Kits

Kits comprising a compound of the invention, or a salt or solvate thereof, and suitable packaging are provided. In one embodiment, a kit further comprises instructions for use. In one aspect, a kit comprises a compound of the invention, or a salt or solvate thereof, and instructions for use of the compounds in the treatment of a disease or condition for which a reduction in basophil and/or mast cell is expected to be or is beneficial.

The invention further provides kits for carrying out the methods of the invention, which comprises one or more compounds described herein or a pharmacological composition comprising a compound described herein. The kits may employ any of the compounds disclosed herein. In one variation, the kit employs a compound described herein or a pharmaceutically acceptable salt thereof.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 month, 4 month, 5 month, 7 month, 8 month, 9 month, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

### General Synthetic Methods

The compounds of the invention may be prepared by a number of processes as generally described below and more specifically in the Examples hereinafter. In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

The following abbreviations are used herein: thin layer chromatography (TLC); hour (h); minute (min); second (sec); ethanol (EtOH); dimethylsulfoxide (DMSO); N,N-dimethylformamide (DMF); trifluoroacetic acid (TFA); tetrahydrofuran (THF); molar (M); Normal (N); aqueous (aq.); methanol (MeOH); dichloromethane (DCM); ethyl acetate (EtOAc); Retention factor (Rf); room temperature (RT).

Compounds detailed herein may be prepared by those of skill in the art by referral to the General Methods and Examples described below. Synthetic routes to bicyclic heterocyclic compounds analogous to compounds C, N, S, W, and AC, in the General Methods below will be familiar to those skilled in the art or readily available in the published literature. As typical examples, routes to functionalized pyrazolopyrimidines, pyridopyrimidines, and pyrazolopyridines are presented in General Methods 1 to 5.

### General Method 1

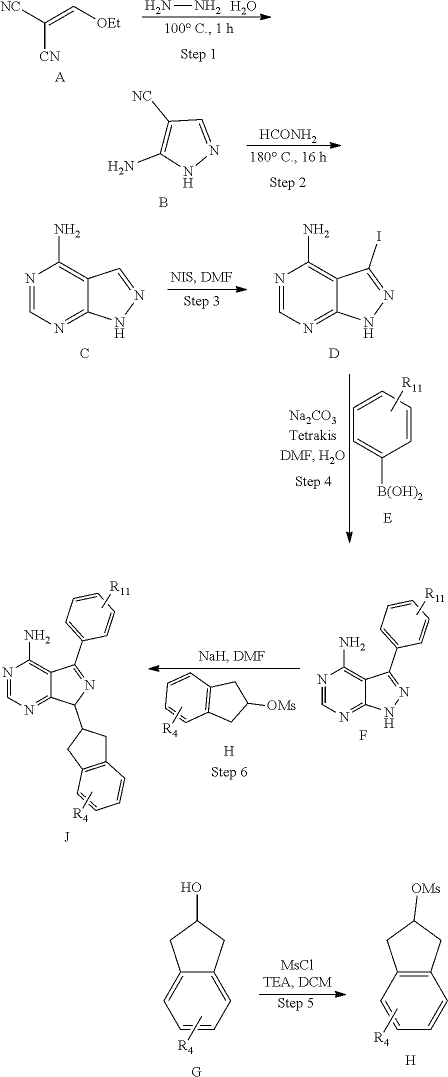

In certain examples of formula (I) provided herein, compounds of the type J can be prepared from starting materials of the type A. This dinitrile A can be treated with hydrazine, under standard pyrazole-forming conditions of step 1 to provide compound B which when treated with formamide in step 2 provides the pyrazolopyrimidine C. Functionalization of the 3-position in step 3 with an appropriate halogenating reagent, such as N-iodosuccinimide (NIS) in this case, provides the 3-iodo derivative D. Other halogenating agents can be envisioned for this step. Treatment of D with an appropriately functionalized boronic acid E under Suzuki coupling conditions of step 4, affords the coupled product F. Separately, appropriately functionalized alcohol reagents of the type G can be converted in step 5 to the labile mesylate intermediate H. Other labile groups such as tosylates and other leaving groups will be familiar to those skilled in the art. Treatment of amine F with mesylate H under basic conditions in final step 6 allows coupling at the 1-position of the pyrazolopyrimidine, to yield the final product J.

### General Method 2

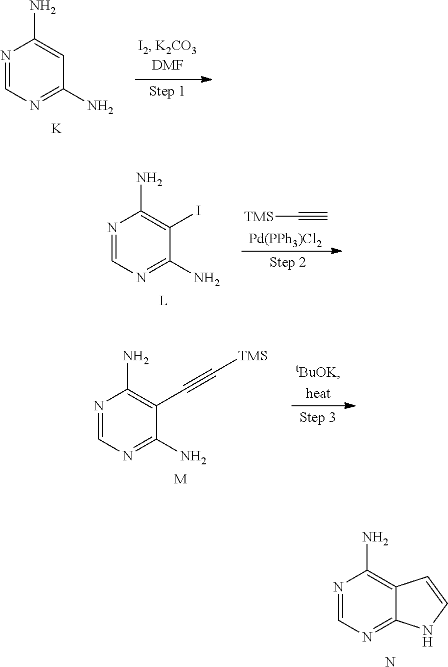

In other examples of formula (I), pyrrolopyrimidines of the type N can be prepared from diaminopyrimidines K. Iodination of compound K under mildly basic conditions of step 1 allows for the iodo intermediate L which, when treated with trimethylsilyl-acetylene under Palladium-mediated coupling conditions of step 2 provides coupled compound M. Base-mediated cyclization of the acetylene in step 3 yields the pyrrolopyrimidine product N. This compound N can be further utilized in a similar fashion to compound C of General Method 1.

### General Method 3

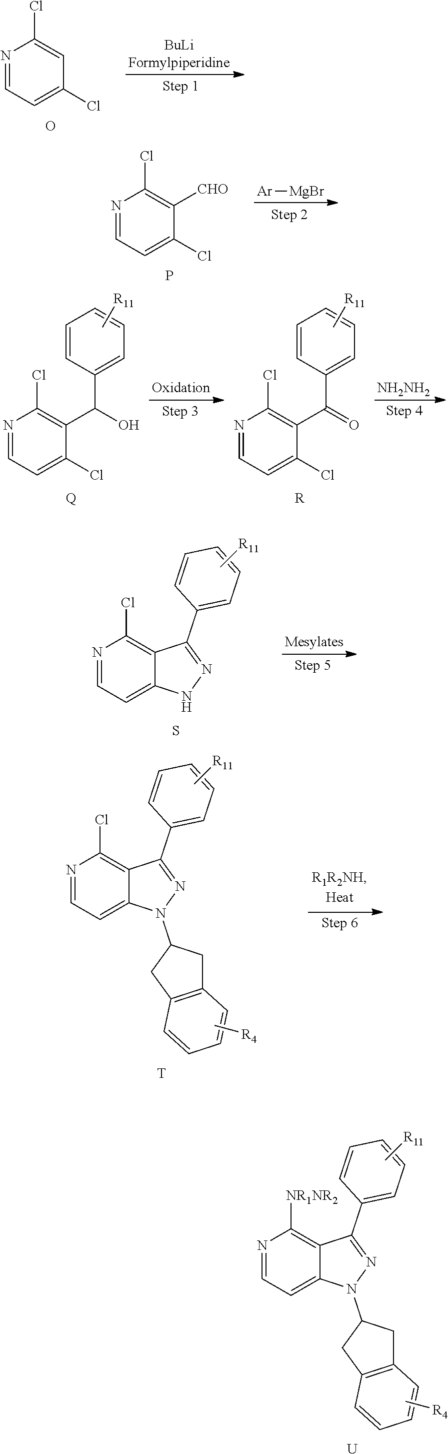

In other examples of formula (I), subjecting 2,4-dichloropyridine 0 to formylation conditions in step 1 provides the aldehyde intermediate P which, when treated in step 2 with appropriately functionalized aryl organometal reagents, such as an aryl Grignard reagent, yields the coupled alcohol Q. Oxidation of the alcohol to the ketone under standard conditions in step 3 affords compound R which, when treated with hydrazine in step 4 gives the pyrazolopyridine S. The free amine group can be coupled in step 5 with various sulfonates, such as the mesylates as described in General Method 1 to give compound T, followed finally by conversion of the chloro group to the desired amine in step 6, to give the desired final product U. Depending on the nature of the substituents in R₄ and R₁₁, protecting groups may be employed followed by the removal of the protecting groups, as needed.

### General Method 4

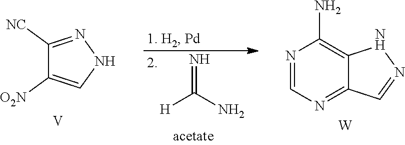

In certain examples of formula (II) provided herein, compounds of the type W can be prepared from starting materials of the type V. Hydrogenation of the nitro pyrazolecarbonitrile V, followed by heating with formamidine acetate provides the pyrazolopyrimidine-7-amine W.

### General Method 5

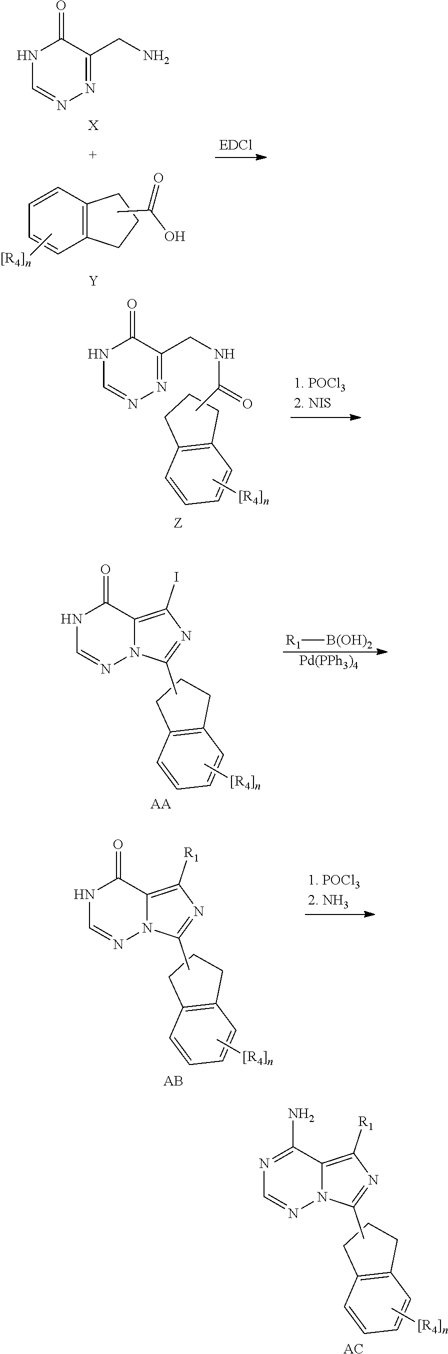

In certain examples of formula (III) provided herein, compounds of the type AC can be prepared from starting materials of the type X. Aminomethyl-triazinone X, described in patent publication US2008/0076921A1, can be coupled with appropriately functionalized carboxylic acid Y, under standard coupling conditions to give amide Z. Chlorination of Z with POCl₃ results in ring closure to yield the imidazotriazinone which, upon treatment with NIS results in the iodinated derivative AA. Coupling of the iodo compound AA with an appropriately functionalized boronic acid R₁—B(OH)₂, under Suzuki coupling conditions, yields the coupled product AB, the pyrimidone moiety of which can be converted to the amino pyrimidine product following well-versed chlorination-amination steps to afford the final product AC.

The following Examples are provided to illustrate but not to limit the invention.

All references disclosed herein are incorporated herein by reference in their entireties.

### EXAMPLES

### Example 1: Preparation of Compound Nos. 1, 1a and 1b

### Step-1: Synthesis of 3-(3,4-dimethoxyphenyl)-1-(1,2,3,4-tetrahydronaphthalen-2-yl)-1H-pyrazolo[3,4-d]pyrimidin-4-amine

See Example No. 17.

### Step-2: Synthesis of 4-(4-amino-1-tetralin-2-yl-pyrazolo[3,4-d]pyrimidin-3-yl)benzene-1,2-diol

To a stirred solution of 3-(3,4-dimethoxyphenyl)-1-tetralin-2-yl-pyrazolo[3,4-d]pyrimidin-4-amine (40 mg, 0.099 mmol) in DCM (5 mL) was added a 1M solution of boron tribromide in DCM (0.5 mL, 0.5 mmol) dropwise under nitrogen atmosphere at RT. The resultant reaction mixture was stirred for 3 h. The reaction was monitored by LCMS. After completion of reaction, the mixture was concentrated under reduced pressure. To the residue was added a saturated solution of sodium bicarbonate solution (10 mL) and the product was extracted with EtOAc (2×20 mL). The combined organic layer was again washed with water (20 mL) and finally with brine solution (20 mL). The organic layer was separated and dried over anhydrous sodium sulfate. Removal of EtOAc under reduced pressure afforded a light brown product that was purified by precipitating in a DCM-Pentane system. The solid was further washed with diethyl ether and dried under vacuum to afford 4-(4-amino-1-tetralin-2-yl-pyrazolo[3,4-d]pyrimidin-3-yl)benzene-1,2-diol (22 mg) as an off-white solid. LCMS: 374.3 (M+1). ¹H NMR (400 MHz, DMSO-d6) δ (ppm): 8.22 (s, 1H), 7.20-7.10 (m, 3H), 7.03 (s, 1H), 6.85 (dd, J=20.0, 11.4 Hz, 2H), 6.68 (d, J=7.7 Hz, 1H), 5.05 (tq, J=14.5, 10.0, 7.1 Hz, 1H), 3.56-3.15 (m, 2H), 2.99 (qd, J=13.8, 12.9, 5.6 Hz, 2H), 2.43-2.29 (m, 1H), 2.26-2.10 (m, 1H). Separation by chiral HPLC affords Compound Nos. 1a and 1b.

### Example 2: Preparation of Compound No. 2

### Step-1: Synthesis of 1-indan-2-yl-3-iodo-pyrazolo[3,4-d]pyrimidin-4-amine

See Example No. 19.

### Step-2: Synthesis of 3-(4-bromo-3-methoxyphenyl)-1-(2,3-dihydro-1H-inden-2-yl)-1H-pyrazolo[3,4-d]pyrimidin-4-amine

Na₂CO₃ (126.4 mg, 1.19 mmol) dissolved in water (4 mL) was added to a solution of 1-indan-2-yl-3-iodo-pyrazolo[3,4-d]pyrimidin-4-amine (150 mg, 0.397 mmol) and (4-bromo-3-methoxy-phenyl)boronic acid (137.7 mg, 0.596 mmol) in DMF (4 mL), followed by addition of tetrakis(triphenylphosphine)palladium(0) (22.97 mg, 0.019 mmol). The reaction was heated at 75° C. for 1 h, the reaction monitored by LCMS. The mixture was diluted with water (50 mL) and extracted with EtOAc (3×50 mL). The combined organic layer was dried over anhydrous sodium sulfate and concentrated under reduced pressure to afford a crude product, which was triturated with acetone (10 mL) and then with pentane (5 mL) to afford 115 mg of 3-(4-bromo-3-methoxy-phenyl)-1-indan-2-yl-pyrazolo[3,4-d]pyrimidin-4-amine as an off-white solid.

### Step-3: Synthesis of 5-(4-amino-1-(2,3-dihydro-1H-inden-2-yl)-1H-pyrazolo[3,4-d]pyrimidin-3-yl)-2-bromophenol

BBr₃ (1 M in DCM, 0.685 mL) was added dropwise at 0° C. to a solution of 3-(4-bromo-3-methoxy-phenyl)-1-indan-2-yl-pyrazolo[3,4-d]pyrimidin-4-amine (60 mg, 0.137 mmol) in DCM (10 mL). The reaction was allowed to warm to RT, and stirred overnight, the reaction monitored by TLC. The reaction was quenched with aq. sodium bicarbonate solution (10 mL) and extracted with EtOAc (2×15 mL). The combined organic layer was dried over anhydrous sodium sulfate and concentrated under reduced pressure to afford a crude product, which was purified by reverse phase preparative HPLC to obtain 16.5 mg of 5-(4-amino-1-indan-2-yl-pyrazolo[3,4-d]pyrimidin-3-yl)-2-bromo-phenol as a white solid (TFA salt). ¹H NMR (400 MHz, DMSO-d₆) δ (ppm): 10.54 (s, 2H), 8.30 (s, 1H), 7.60 (d, J=8.2 Hz, 1H), 7.32-7.24 (m, 2H), 7.24-7.12 (m, 3H), 6.99 (d, J=7.6 Hz, 1H), 5.73 (p, J=7.9 Hz, 1H), 3.48 (m, 4H)

### Example 3: Preparation of Compound No. 3

### Step-1: Synthesis of 3-(3-fluoro-5-methoxy-phenyl)-1H-pyrazolo[3,4-d]pyrimidin-4-amine

See Example No. 6.

### Step-2: Synthesis of 2,3-dihydro-1H-inden-2-yl methanesulfonate

To a solution of indan-2-ol (250 mg, 1.863 mmol) in DCM (5 mL) was added triethylamine (1.29 mL, 9.268 mmol) at 0° C. This was followed by slow addition of mesyl chloride (0.288 mL, 3.72 mmol). The reaction was allowed to stir at 0° C. for 2 h, and monitored by ¹H NMR. After completion of reaction, the mixture was quenched with water (25 mL) and extracted with DCM (100 mL). The organic layer was dried over anhydrous sodium sulfate and concentrated under reduced pressure to obtain 500 mg of indan-2-yl methanesulfonate as a brown solid.

### Step-3: Synthesis of 1-(2,3-dihydro-1H-inden-2-yl)-3-(3-fluoro-5-methoxyphenyl)-1H-pyrazolo[3,4-d]pyrimidin-4-amine

Sodium hydride (60% suspension in mineral oil, 101 mg, 2.314 mmol) was added portionwise to a solution of 3-(3-fluoro-5-methoxy-phenyl)-1H-pyrazolo[3,4-d]pyrimidin-4-amine (150 mg, 0.578 mmol) in DMF (3 mL) at 0° C. The reaction was allowed to warm to RT and stirred for 30 min, followed by dropwise addition of indan-2-yl methanesulfonate (245 mg, 1.154 mmol, in 1 mL DMF) at 0° C. The reaction was heated at 80° C. for 2 h. The reaction was monitored by TLC. The reaction was quenched with ice cold water (20 mL) and extracted with EtOAc (2×25 mL). The combined organic layers were washed with water (30 mL) and brine (30 mL), dried over anhydrous sodium sulfate and concentrated under reduced pressure to obtain the crude product which was purified by flash chromatography using 20% EtOAc in hexane as eluent to obtain 20 mg of 3-(3-fluoro-5-methoxy-phenyl)-1-indan-2-yl-pyrazolo[3,4-d]pyrimidin-4-amine as a brown solid.

### Step-4: Synthesis of 3-(4-amino-1-(2,3-dihydro-1H-inden-2-yl)-1H-pyrazolo[3,4-d]pyrimidin-3-yl)-5-fluorophenol

BBr₃ (1M in DCM, 0.16 mL) was added dropwise at −50° C. to a solution of 3-(3-fluoro-5-methoxy-phenyl)-1-indan-2-yl-pyrazolo[3,4-d]pyrimidin-4-amine (20 mg, 0.053 mmol) in DCM (2 mL). The reaction was allowed to warm to RT, and stirred overnight. The reaction was monitored by TLC. The reaction was quenched with 1M aq. HCl (5 mL) and extracted with DCM (2×10 mL). The combined organic layer was dried over anhydrous sodium sulfate and concentrated under reduced pressure to afford a crude product, which was purified by reverse phase preparative HPLC to obtain 3 mg of 3-(4-amino-1-indan-2-yl-pyrazolo[3,4-d]pyrimidin-3-yl)-5-fluoro-phenol as a white solid. ¹HNMR (400 MHz, CDCl₃) δ (ppm): 8.40 (s, 1H), 7.24-7.16 (m, 4H), 6.98 (d, J=9.9 Hz, 1H), 6.92 (s, 1H), 6.68 (d, J=9.9 Hz, 1H), 5.83 (p, J=8.3 Hz, 1H), 5.56 (s, 2H), 3.68 (dd, J=15.8, 8.3 Hz, 2H), 3.48 (dd, J=15.8, 8.5 Hz, 2H).

### Example 4: Preparation of Compound Nos. 4, 4a and 4b

### Step-1: Synthesis of 3-(3-fluoro-5-methoxy-phenyl)-1H-pyrazolo[3,4-d]pyrimidin-4-amine

See Example No. 6.

### Step-2: Synthesis of 2,3-dihydro-1H-inden-1-yl methanesulfonate

To a solution of indan-1-ol (250 mg, 1.86 mmol) in DCM (6 mL) was added triethylamine (1.29 mL, 9.31 mmol) dropwise at 0° C. To this was added mesyl chloride (0.288 mL, 3.72 mmol) slowly. The reaction was allowed to stir at 0° C. for 1.5 h and monitored by ¹HNMR. After completion of reaction, the mixture was quenched with water (25 mL) and extracted with DCM (100 mL). The organic layer was washed with brine, dried over anhydrous sodium sulfate and concentrated under reduced pressure to obtain 260 mg of indan-1-yl methanesulfonate as a yellow liquid.

### Step-3: Synthesis of 1-(2,3-dihydro-1H-inden-1-yl)-3-(3-fluoro-5-methoxyphenyl)-1H-pyrazolo[3,4-d]pyrimidin-4-amine

Sodium hydride (60% suspension in mineral oil, 192 mg, 4.640 mmol) was added portionwise to a solution of 3-(3-fluoro-5-methoxy-phenyl)-1H-pyrazolo[3,4-d]pyrimidin-4-amine (300 mg, 1.157 mmol) in DMF (6 mL) at 0° C. The reaction was allowed to warm to RT and stirred for 30 min, followed by dropwise addition of indan-2-yl methanesulfonate (491 mg, 2.313 mmol, in 2 mL DMF) at 0° C. The mixture was heated at 80° C. for 2 h and monitored by TLC. The reaction was quenched with ice cold water (30 mL) and extracted with EtOAc (2×40 mL). The combined organic layers were washed with water (50 mL) and brine (40 mL), dried over anhydrous sodium sulfate and concentrated under reduced pressure to obtain the crude product which was purified by flash chromatography using 60% EtOAc in hexane as eluent to obtain 110 mg of 3-(3-fluoro-5-methoxy-phenyl)-1-indan-1-yl-pyrazolo[3,4-d]pyrimidin-4-amine as a white solid. ¹HNMR (400 MHz, CDCl₃) δ (ppm): 8.44 (s, 1H), 7.32 (d, J=7.6 Hz, 1H), 7.24 (d, J=7.4 Hz, 1H), 7.13 (t, J=7.4 Hz, 1H), 7.04 (d, J=7.5 Hz, 1H), 6.96 (m, 2H), 6.68 (dt, J=10.5, 2.5 Hz, 1H), 6.54 (t, J=7.5 Hz, 1H), 5.51 (s, 2H), 3.83 (s, 3H), 3.36 (ddd, J=15.9, 8.3, 4.9 Hz, 1H), 3.06 (dt, J=15.7, 7.8 Hz, 1H), 2.84-2.64 (m, 2H). Separation by chiral HPLC affords Compound Nos. 4a and 4b.

### Example 5: Preparation of Compound No. 5

### Step-1: Synthesis of 3-(3-fluoro-5-methoxy-phenyl)-1H-pyrazolo[3,4-d]pyrimidin-4-amine

See Example No. 3.

### Step-2: Synthesis of 2,3-dihydro-1H-inden-2-yl methanesulfonate

To a solution of indan-2-ol (250 mg, 1.863 mmol) in DCM (5 mL) was added triethylamine (1.29 mL, 9.268 mmol) at 0° C. This was followed by slow addition of mesyl chloride (0.288 mL, 3.72 mmol). The reaction was allowed to stir at 0° C. for 2 h, and monitored by ¹HNMR. After completion of reaction, the mixture was quenched with water (25 mL) and extracted with DCM (100 mL). The organic layer was dried over anhydrous sodium sulfate and concentrated under reduced pressure to obtain 500 mg of indan-2-yl methanesulfonate as a brown solid.

### Step-3: Synthesis of 1-(2,3-dihydro-1H-inden-2-yl)-3-(3-fluoro-5-methoxyphenyl)-1H-pyrazolo[3,4-d]pyrimidin-4-amine

Sodium hydride (60% suspension in mineral oil, 101 mg, 2.314 mmol) was added portionwise to a solution of 3-(3-fluoro-5-methoxy-phenyl)-1H-pyrazolo[3,4-d]pyrimidin-4-amine (150 mg, 0.578 mmol) in DMF (3 mL) at 0° C. The reaction was allowed to warm to RT and stirred for 30 min, followed by dropwise addition of indan-2-yl methanesulfonate (245 mg, 1.154 mmol, in 1 mL DMF) at 0° C. The reaction was heated at 80° C. for 2 h. The reaction was monitored by TLC. The reaction was quenched with ice cold water (20 mL) and extracted with EtOAc (2×25 mL). The combined organic layers were washed with water (30 mL) and brine (30 mL), dried over anhydrous sodium sulfate and concentrated under reduced pressure to obtain the crude product which was purified by flash chromatography using 20% EtOAc in hexane as eluent to obtain 20 mg of 3-(3-fluoro-5-methoxy-phenyl)-1-indan-2-yl-pyrazolo[3,4-d]pyrimidin-4-amine as a brown solid. ¹HNMR (400 MHz, CDCl₃) δ (ppm): 8.40 (s, 1H), 7.21 (d, J=4.7 Hz, 4H), 7.00 (m, 2H), 6.71 (d, J=10.5 Hz, 1H), 5.87-5.77 (m, 1H), 5.58 (s, 2H), 3.86 (s, 3H), 3.71 (dd, J=15.8, 8.5 Hz, 2H), 3.49 (dd, J=15.6, 8.8 Hz, 2H).

### Example 6: Preparation of Compound Nos. 6, 6a and 6b

### Step-1: Synthesis of 5-amino-1H-pyrazole-4-carbonitrile

To 2-(ethoxymethylene)propanedinitrile (20 g, 163.80 mmol) was added hydrazine hydrate (15.9 mL, 327.60 mmol) dropwise at 0° C. and the resultant reaction mixture was heated at 100° C. in a closed reagent bottle for 1 h. The reaction was monitored by TLC. After completion of reaction, the mixture was cooled to RT and water (50 mL) was added to the reaction mixture. The product was extracted using EtOAc (3×200 mL). The combined organic layer was dried over anhydrous sodium sulfate and concentrated under reduced pressure to obtain 15 g of 5-amino-1H-pyrazole-4-carbonitrile as a light brown solid.

### Step-2: Synthesis of 1H-pyrazolo[3,4-d]pyrimidin-4-amine

To 5-amino-1H-pyrazole-4-carbonitrile (15 g, 138.76 m mol) was added formamide (75 mL) under nitrogen atmosphere and the reaction mixture was heated at 180° C. overnight. The reaction was monitored by TLC. After completion of reaction, the mixture was cooled to 0° C. and water (50 mL) was added, and a precipitate formed. The precipitate was collected by filtration and dried to obtain 13.1 g of 1H-pyrazolo[3,4-d]pyrimidin-4-amine as a yellow solid.

### Step-3: Synthesis of 3-iodo-1H-pyrazolo[3,4-d]pyrimidin-4-amine

To a suspension of 1H-pyrazolo[3,4-d]pyrimidin-4-amine (7.5 g, 55.50 mmol) in anhydrous DMF (50 mL) was added N-iodosuccinimide (49 g, 222.0 mmol) portionwise under nitrogen atmosphere. The reaction mixture was heated at 80° C. for 2 h. The reaction was monitored by TLC. After completion of reaction, the mixture was cooled to RT and diluted with EtOAc (400 mL). Saturated aq. sodium thiosulfate solution (100 mL) was added, and a precipitate formed. The precipitate was collected by filtration, washed with additional amount of water, diethyl ether and then dried to obtain 14 g of 3-iodo-1H-pyrazolo[3,4-d]pyrimidin-4-amine as an off-white solid.

### Step-4: Synthesis of 3-(3-fluoro-5-methoxyphenyl)-1H-pyrazolo[3,4-d]pyrimidin-4-amine

To a solution of 3-iodo-1H-pyrazolo[3,4-d]pyrimidin-4-amine (700 mg, 2.68 mmol) in DMF (12 mL) was added 3-fluoro-5-methoxyphenylboronic acid (546.9 mg, 3.21 mmol) at RT. Then, sodium carbonate (852.6 mg, 8.04 mmol) dissolved in water (10 mL) was added to the reaction mixture followed by addition of Pd(PPh₃)₄ (309.8 mg, 0.268 mmol) at RT and the resultant reaction mixture was heated at 100° C. for 16 h. The reaction was monitored by TLC and through LCMS. After completion of reaction, the reaction mixture was filtered through celite. The filtrate obtained was diluted with EtOAc (300 mL). The organic layer was washed with water (2×150 mL), dried over sodium sulfate and concentrated. The crude compound was purified by column chromatography (silica gel, 100-200) with 4% MeOH/DCM as eluent to afford 225 mg of 3-(3-fluoro-5-methoxyphenyl)-1H-pyrazolo[3,4-d]pyrimidin-4-amine as a light brown solid.

### Step-5: Synthesis of 1,2,3,4-tetrahydronaphthalen-2-ol

To a stirred solution of 3,4-dihydronaphthalen-2(1H)-one (500 mg, 3.420 mmol) in MeOH (5 mL) was added NaBH₄ (259.0 mg, 6.84 mmol) at 0° C. portionwise. The reaction mixture was then stirred at RT for 1 h. The reaction was monitored by TLC. After completion of reaction, the mixture was quenched with ice and concentrated under reduced pressure. The residue obtained was diluted with water (150 mL) and extracted with EtOAc (2×200 mL). The combined organic layer was dried over sodium sulfate and concentrated to afford 500 mg of 1,2,3,4-tetrahydronaphthalen-2-ol as a yellow semi-solid, which was taken to the next step without further purification.

### Step-6: Synthesis of 1,2,3,4-tetrahydronaphthalen-2-yl methanesulfonate

To a stirred solution of 1,2,3,4-tetrahydronaphthalen-2-ol (250 mg, 1.68 mmol) in DCM (5 mL) was added triethylamine (1.17 mL, 8.50 mmol) at 0° C. followed by addition of mesyl chloride (0.26 mL, 3.35 mmol). The reaction mixture was then stirred at 0° C. for 2 h. The reaction was monitored by ¹H NMR. After completion of reaction, the mixture was quenched with ice, and extracted with DCM (2×50 mL). The combined organic layer was dried over sodium sulfate and concentrated to afford 340 mg of 1,2,3,4-tetrahydronaphthalen-2-yl methanesulfonate as a brown liquid which was taken to the next step without further purification.

### Step-7: Synthesis of 3-(3-fluoro-5-methoxyphenyl)-1-(1,2,3,4-tetrahydronaphthalen-2-yl)-1H-pyrazolo[3,4-d]pyrimidin-4-amine

To a stirred solution of 3-(3-fluoro-5-methoxyphenyl)-1H-pyrazolo[3,4-d]pyrimidin-4-amine (300 mg, 1.15 mmol) in DMF (5.5 mL) was added sodium hydride (60% suspension in mineral oil; 92.5 mg, 2.31 mmol) at 0° C. slowly, and the reaction mixture was stirred at RT for 30 min. Then, 1,2,3,4-tetrahydronaphthalen-2-yl methanesulfonate (92.8 mg, 1.73 mmol) dissolved in DMF (2.5 mL) was added dropwise to the reaction mixture at 0° C. and the resultant mixture was heated at 80° C. for 1 h. The reaction was monitored by TLC. After 1 h, the reaction mixture was cooled to RT and diluted with EtOAc (100 mL). The organic layer was washed with water (2×50 mL), brine (40 mL), dried over sodium sulfate and concentrated. The crude compound was purified by preparative HPLC afford 5 mg of 3-(3-fluoro-5-methoxyphenyl)-1-(1,2,3,4-tetrahydronaphthalen-2-yl)-1H-pyrazolo[3,4-d]pyrimidin-4-amine as a white solid.

### Step-8: Synthesis of 3-(4-amino-1-(1,2,3,4-tetrahydronaphthalen-2-yl)-1H-pyrazolo[3,4-d]pyrimidin-3-yl)-5-fluorophenol

To a stirred solution of 3-(3-fluoro-5-methoxyphenyl)-1-(1,2,3,4-tetrahydronaphthalen-2-yl)-1H-pyrazolo[3,4-d]pyrimidin-4-amine (45 mg, 0.115 mmol) in DCM (2 mL) was added BBr₃ (1M solution in DCM; 0.57 mL, 0.577 mmol) dropwise at −50° C., and the resultant reaction mixture was gradually warmed to RT over a period of 5 h. The reaction was monitored by TLC. After completion of reaction, the mixture was slowly quenched at 0° C. using 1M-HCl, and then extracted with DCM (100 mL). The combined organic layers were washed with water (50 mL), dried over sodium sulfate and concentrated. The crude compound was purified by preparative HPLC affording 4.8 mg of 3-(4-amino-1-(1,2,3,4-tetrahydronaphthalen-2-yl)-1H-pyrazolo[3,4-d]pyrimidin-3-yl)-5-fluorophenol as a white solid. Separation by chiral HPLC provided Compound Nos. 6a and 6b. Compound No. 6: ¹H NMR (400 MHz, CD₃OD) δ (ppm): 8.35 (s, 3H), 7.18-7.08 (m, 12H), 6.97-6.87 (m, 5H), 6.69 (d, J=10.6 Hz, 3H), 5.23 (tt, J=11.1, 4.3 Hz, 3H), 3.54 (dd, J=16.1, 11.1 Hz, 1H), 3.14 (m, 3H), 2.49 (qd, J=11.8, 6.6 Hz, 1H), 2.30 (td, J=12.7, 7.7 Hz, 1H. Compound No. 6a: ¹H NMR (400 MHz, CDCl₃) δ (ppm): 8.39 (s, 1H), 7.14-6.96 (m, 6H), 6.69 (m, 1H), 5.53 (bs, 2H), 5.31-5.10 (m, 1H), 3.62 (dd, J=16.1, 11.3 Hz, 1H), 3.19-3.00 (m, 3H), 2.51 (qd, J=12.1, 6.1 Hz, 1H), 2.29 (m, 1H). Compound No. 6b: ¹H NMR (400 MHz, CDCl₃) δ (ppm): 8.39 (s, 1H), 7.20 (s, 1H), 7.18-6.94 (m, 5H), 6.69 (d, J=9.9 Hz, 1H), 5.53 (bs, 2H), 5.23 (m, 1H), 3.62 (dd, J=16.1, 11.3 Hz, 1H), 3.26-3.00 (m, 3H), 2.51 (qd, J=12.1, 6.0 Hz, 1H), 2.30 (m, 1H).

### Example 7: Preparation of Compound Nos. 7, 7a and 7b

### Synthesis of 3-(3-fluoro-5-methoxyphenyl)-1-(1,2,3,4-tetrahydronaphthalen-2-yl)-1H-pyrazolo[3,4-d]pyrimidin-4-amine

The title compound was prepared following Steps 1-7 of Example 6. ¹H NMR (400 MHz, CDCl₃) δ (ppm): 8.39 (s, 1H), 7.13 (d, J=17.4 Hz, 4H), 7.03 (d, J=9.1 Hz, 2H), 6.72 (d, J=10.5 Hz, 1H), 5.60 (s, 2H), 5.23 (td, J=11.4, 10.6, 5.7 Hz, 1H), 3.87 (s, 3H), 3.64 (dd, J=16.1, 11.3 Hz, 1H), 3.27-3.00 (m, 3H), 2.53 (qd, J=12.1, 5.9 Hz, 1H), 2.31 (d, J=12.9 Hz, 1H). Separation by chiral HPLC provides Compound Nos. 7a and 7b.

### Example 8: Preparation of Compound Nos. 8, 8a and 8b

### Step-1: Synthesis of 3-iodo-1-(1,2,3,4-tetrahydronaphthalen-2-yl)-1H-pyrazolo[3,4-d]pyrimidin-4-amine

See Example No. 19.

### Step-2: Synthesis of (4-bromo-3-methoxyphenyl)boronic acid

To a stirred solution of 1-bromo-4-iodo-2-methoxybenzene (2 g, 6.39 mmol) in diethyl ether (20 mL) was added n-BuLi (1.3 M in hexane; 5.4 mL, 7.03 mmol) at −78° C. dropwise. The reaction was allowed to stir at −78° C. for 30 min, and then triisopropyl borate (1.62 mL, 7.03 mmol) was slowly added to the reaction mixture at −78° C. The temperature of the reaction mixture was gradually raised to RT over a period of 1 h, and then the reaction was stirred at RT for 30 min. After 30 min, 3N-HCl was slowly added to the reaction mixture at 0° C. and the reaction was stirred for further 1 h. The reaction was monitored by TLC. After completion of reaction, the mixture was diluted with water (150 mL) and extracted with EtOAc (2×200 mL). The combined organic layers were washed with brine (100 mL), dried over sodium sulfate and concentrated. The residue obtained was triturated with hexane (2×25 mL) to afford 1.1 g of 4-bromo-3-methoxyphenylboronic acid as a white solid.

### Step-3: Synthesis of 3-(4-bromo-3-methoxyphenyl)-1-(1,2,3,4-tetrahydronaphthalen-2-yl)-1H-pyrazolo[3,4-d]pyrimidin-4-amine

To a solution of 3-iodo-1-(1,2,3,4-tetrahydronaphthalen-2-yl)-1H-pyrazolo[3,4-d]pyrimidin-4-amine (150 mg, 0.383 mmol) in DMF (4 mL) was added 4-bromo-3-methoxyphenylboronic acid (132.7 mg, 0.575 mmol) at RT. Then, Na₂CO₃ (121.90 mg, 1.15 mmol) dissolved in water (4 mL) was added to the reaction mixture followed by addition of Pd(PPh₃)₄ (22.15 mg, 0.019 mmol) at RT and the resultant reaction mixture was heated at 70° C. for 1.5 h. The reaction was monitored through LCMS. After completion of reaction, the mixture was diluted with water (100 mL) and extracted with EtOAc (2×150 mL). The combined organic layers were washed with brine (50 mL), dried over sodium sulfate and concentrated. The residue obtained was triturated with diethyl ether (2×10 mL) to afford 125 mg of 5-(4-amino-1-(1,2,3,4-tetrahydronaphthalen-2-yl)-1H-pyrazolo[3,4-d]pyrimidin-3-yl)-2-bromophenol as a yellow solid.

### Step-4: Synthesis of 5-(4-amino-1-(1,2,3,4-tetrahydronaphthalen-2-yl)-1H-pyrazolo[3,4-d]pyrimidin-3-yl)-2-bromophenol

To a stirred solution of 5-(4-amino-1-(1, 2, 3, 4-tetrahydronaphthalen-2-yl)-1H-pyrazolo[3,4-d]pyrimidin-3-yl)-2-bromophenol (70 mg, 0.155 mmol) in DCM (4 mL) was added BBr₃ (1M in DCM; 0.62 mL, 0.621 mmol) at 0° C. dropwise and the resultant reaction mixture was stirred at RT for 7.5 h. The reaction was monitored by TLC. After completion of reaction, the reaction mixture was slowly quenched using saturated solution of sodium bicarbonate (15 mL) and extracted with EtOAc (100 mL). The organic layer was washed with water (40 mL), brine (30 mL), dried over sodium sulfate and concentrated. The crude compound was purified by preparative HPLC to afford 8.5 mg of 5-(4-amino-1-(1, 2, 3, 4-tetrahydronaphthalen-2-yl)-1H-pyrazolo[3,4-d]pyrimidin-3-yl)-2-bromophenol as an off-white solid. ¹H NMR (400 MHz, DMSO-d6) δ (ppm): 8.25 (s, 1H), 7.62 (d, J=8.1 Hz, 1H), 7.26 (s, 1H), 7.14 (d, J=10.2 Hz, 4H), 7.02 (d, J=8.5 Hz, 1H), 5.09 (q, J=7.7 Hz, 1H), 3.16 (dd, J=16.2, 5.8 Hz, 1H), 3.02 (d, J=16.5 Hz, 2H), 2.38-2.26 (m, 1H). Separation by chiral HPLC provides Compound Nos. 8a and 8b.

### Example 9: Preparation of Compound Nos. 9, 9a and 9b

### Step-1: Synthesis of 3-iodo-1-tetralin-2-yl-pyrazolo[3,4-d]pyrimidin-4-amine

See Example No. 19.

### Step-2: Synthesis of 3-(1H-indazol-6-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (150 mg, 0.383 mmol) and 1H-indazole-6-boronic acid (93 mg, 0.575 mmol) in DMF (3 mL) was added a solution of sodium carbonate (81 mg, 0.766 mmol) in water (3 mL) followed by the addition of tetrakis(triphenylphosphine) palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated under reduced pressure to obtain a crude product which was purified by reverse phase preparative HPLC to obtain 23 mg of 3-(1H-indazol-6-yl)-1-tetralin-2-yl-pyrazolo[3,4-d]pyrimidin-4-amine as a white solid (Racemate). ¹H NMR (400 MHz, DMSO-d₆) δ (ppm): 13.22 (s, 1H), 8.28 (s, 1H), 8.16 (s, 1H), 7.92 (d, J=8.3 Hz, 1H), 7.79 (s, 1H), 7.45 (d, J=8.3 Hz, 1H), 7.15 (m, 4H), 5.13 (m, 1H), 3.53-3.37 (m, 1H), 3.32-3.14 (m, 1H), 3.04 (m, 2H), 2.38 (m, 1H), 2.28 (m, 1H). Separation by chiral HPLC provides Compound Nos. 9a and 9b.

### Example 10: Preparation of Compound Nos. 10, 10a and 10b

### Step-1: Synthesis of 1-indan-1-yl-3-iodo-pyrazolo[3,4-d]pyrimidin-4-amine

See Example No. 19.

### Step-2: Synthesis of 5-(4-amino-1-indan-1-yl-pyrazolo[3,4-d]pyrimidin-3-yl)-2-fluoro-phenol

To a solution of 1-indan-1-yl-3-iodo-pyrazolo[3,4-d]pyrimidin-4-amine (200 mg, 0.530 mmol) and (4-fluoro-3-hydroxy-phenyl)boronic acid (124 mg, 0.795 mmol) in DMF (3 mL) was added a solution of sodium carbonate (112 mg, 1.060 mmol) in water (3 mL) followed by the addition of tetrakis(triphenylphosphine)palladium(0) (61 mg, 0.0530 mmol). The reaction mixture was heated in a reagent bottle at 100° C. overnight. The reaction was monitored by TLC. After completion of reaction, water (20 mL) was added to the reaction mixture and the product was extracted with EtOAc (2×20 mL). The combined organic layer was again washed with water (2×15 mL) and finally with brine solution (15 mL). The organic layer was separated, dried over anhydrous sodium sulfate. Removal of EtOAc under reduced pressure afforded a crude product that was purified by reverse phase preparative HPLC to afford 5-(4-amino-1-indan-1-yl-pyrazolo[3,4-d]pyrimidin-3-yl)-2-fluoro-phenol (73 mg) as an off-white solid. LCMS: 361.9 (M+1). ¹H NMR (400 MHz, DMSO-d6) δ (ppm): 8.25 (s, 1H), 7.32 (d, J=7.5 Hz, 1H), 7.23 (dd, J=12.0, 7.9 Hz, 2H), 7.09 (t, J=8.0 Hz, 2H), 6.96 (dt, J=7.2, 3.1 Hz, 1H), 6.87 (d, J=7.5 Hz, 1H), 6.37 (t, J=7.4 Hz, 1H), 3.24-3.09 (m, 1H), 2.98 (dt, J=15.8, 7.8 Hz, 1H), 2.62-2.50 (m, 2H). Separation by chiral HPLC provides Compound Nos. 10a and 10b.

### Example 11: Preparation of Compound Nos. 11, 11a and 11b

### Step-1: Synthesis of 3-iodo-1-tetralin-1-yl-pyrazolo[3,4-d]pyrimidin-4-amine

See Example No. 19.

### Step-2: Synthesis of (3-fluoro-5-hydroxy-phenyl)boronic acid

To a stirred solution of (3-fluoro-5-methoxy-phenyl)boronic acid (500 mg, 2.942 mmol) in DCM (15 mL) was added 1 M solution of boron tribromide in DCM (14.7 mL, 14.7 mmol) under nitrogen atmosphere at 0° C. The reaction mixture was stirred at RT for 3h. The reaction was monitored by TLC. After completion of reaction, the mixture was concentrated under reduced pressure. Water (15 mL) was added to the residue and the pH of the mixture was adjusted to 2 by the addition of 1M HCl (aq.). The product was extracted with EtOAc (2×25 mL). The combined organic layer was dried over anhydrous sodium sulfate and concentrated under reduced pressure to afford (3-fluoro-5-hydroxy-phenyl)boronic acid (430 mg) as an off-white solid.

### Step-3: Synthesis of 3-(4-amino-1-tetralin-1-yl-pyrazolo[3,4-d]pyrimidin-3-yl)-5-fluoro-phenol

To a solution of 3-iodo-1-tetralin-1-yl-pyrazolo[3,4-d]pyrimidin-4-amine (150 mg, 0.383 mmol) and (3-fluoro-5-hydroxy-phenyl)boronic acid (90 mg, 0.575 mmol) in DMF (3 mL) was added a solution of sodium carbonate (81 mg, 0.766 mmol) in water (3 mL) followed by the addition of tetrakis(triphenylphosphine)palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted with EtOAc (2×20 mL). The combined organic layer was again washed with water (2×15 mL) and finally with brine solution (15 mL). The organic layer was separated, and dried over anhydrous sodium sulfate. Removal of EtOAc under reduced pressure afforded a crude product that was purified by reverse phase preparative HPLC to afford 3-(4-amino-1-tetralin-1-yl-pyrazolo[3,4-d]pyrimidin-3-yl)-5-fluoro-phenol (14.5 mg) as an off-white solid. LCMS: 376.0 (M+1). ¹H NMR (400 MHz, DMSO-d6) δ (ppm): 8.25 (s, 1H), 7.14 (q, J=8.1, 7.7 Hz, 2H), 6.98 (t, J=7.3 Hz, 1H), 6.84-6.71 (m, 2H), 6.63 (dd, J=10.8, 2.6 Hz, 1H), 6.47 (d, J=7.8 Hz, 1H), 6.05 (t, J=7.2 Hz, 1H), 2.96-2.69 (m, 2H), 2.27 (t, J=10.2 Hz, 1H), 2.23-2.07 (m, 2H), 1.84 (m, 1H). Separation by chiral HPLC provides Compound Nos. 11a and 11b.

### Example 12: Preparation of Compound Nos. 12, 12a and 12b

### Step-1: Synthesis of 3-iodo-1-tetralin-1-yl-pyrazolo[3,4-d]pyrimidin-4-amine

See Example No. 19.

### Step-2: Synthesis of (4-fluoro-3-hydroxy-phenyl)boronic acid

To a stirred solution of (4-fluoro-3-methoxy-phenyl)boronic acid (500 mg, 2.942 mmol) in DCM (15 mL) was added a 1M solution of boron tribromide in DCM (14.7 mL, 14.7 mmol) under nitrogen atmosphere at 0° C. The reaction mixture was stirred at RT for 3h. The reaction was monitored by TLC. After completion of reaction, the mixture was concentrated under reduced pressure. Water (15 mL) was added to the residue and the pH of the mixture adjusted to 2 by the addition of 1M HCl (aq.). The product was extracted with EtOAc (2×25 mL). The combined organic layer was dried over anhydrous sodium sulfate and concentrated under reduced pressure to afford (4-fluoro-3-hydroxy-phenyl)boronic acid (440 mg) as an off-white solid.

### Step-3: Synthesis of 5-(4-amino-1-tetralin-1-yl-pyrazolo[3,4-d]pyrimidin-3-yl)-2-fluoro-phenol

To a solution of 3-iodo-1-tetralin-1-yl-pyrazolo[3,4-d]pyrimidin-4-amine (150 mg, 0.383 mmol) and (4-fluoro-3-hydroxy-phenyl)boronic acid (90 mg, 0.575 mmol) in DMF (3 mL) was added a solution of sodium carbonate (81 mg, 0.766 mmol) in water (3 mL) followed by the addition of tetrakis(triphenylphosphine)palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted with EtOAc (2×20 mL). The combined organic layer was again washed with water (2×15 mL) and finally with brine solution (15 mL). The organic layer was separated, and dried over anhydrous sodium sulfate. Removal of EtOAc under reduced pressure afforded a crude product that was purified by reverse phase preparative HPLC to afford 5-(4-amino-1-tetralin-1-yl-pyrazolo[3,4-d]pyrimidin-3-yl)-2-fluoro-phenol (18.9 mg) as an off-white solid. LCMS: 376.0 (M+1). ¹H NMR (400 MHz, DMSO-d6) δ (ppm): 8.25 (s, 1H), 7.23 (t, J=9.7 Hz, 1H), 7.12 (m, 3H), 6.96 (t, J=6.7 Hz, 2H), 6.47 (d, J=7.9 Hz, 1H), 6.17-5.91 (m, 1H), 2.84 (dt, J=34.2, 13.2 Hz, 2H), 2.29 (q, J=10.2, 9.6 Hz, 1H), 2.14 (dd, J=22.2, 11.0 Hz, 2H), 1.93-1.76 (m, 1H). Separation by chiral HPLC provides Compound Nos. 12a and 12b.

### Example 13: Preparation of Compound Nos. 13, 13a and 13b

### Step-1: Synthesis of 3-iodo-1H-pyrazolo[3,4-d]pyrimidin-4-amine

See Example No. 6.

### Step-2: Synthesis of 3-iodo-1-(1,2,3,4-tetrahydronaphthalen-2-yl)-1H-pyrazolo[3,4-d]pyrimidin-4-amine

A solution of 3-iodo-1H-pyrazolo[3,4-d]pyrimidin-4-amine (2G, 7.662 mmol) in 15 mL DMF was cooled to 0° C. To this solution was added sodium hydride (613 mg, 15.324 mmol) under nitrogen atmosphere. The reaction mixture was stirred at the same temperature for 30 min. Then a solution of 1,2,3,4-tetrahydronaphthalen-2-yl methanesulfonate (2.08 g, 9.194 mmol) in 2 mL DMF was added and the reaction mixture was heated at 80° C. for 2 h. The reaction was monitored by TLC. After completion of reaction, the reaction mixture was cooled to 0° C. and quenched by ice cold water. The product was extracted using EtOAc (2×100 mL). The organic layers were washed with brine and dried over sodium sulfate and concentrated to obtain the crude product which was washed with ether and then dried again to obtain 0.38 g of off white solid.

### Step-3: Synthesis of 3-(4-fluorophenyl)-1-(1,2,3,4-tetrahydronaphthalen-2-yl)-1H-pyrazolo[3,4-d]pyrimidin-4-amine

To a suspension of 3-iodo-1-(1,2,3,4-tetrahydronaphthalen-2-yl)-1H-pyrazolo[3,4-d]pyrimidin-4-amine (0.25 g, 0.639 mmol, 1 equiv.) and 4-fluorophenylboronic acid (0.134 g, 0.959 mmol, 1.5 equiv.) in DMF:water (2:1) was added sodium carbonate (0.135 g, 1.278 mmol, 2 equiv.) and tetrakis(triphenylphosphine)palladium(0) (73.8 mg, 0.0639 mmol, 0.1 equiv.) at RT in a reagent bottle. The reagent bottle was sealed and the mixture stirred at 100° C. overnight. The reaction was monitored by TLC. After completion of reaction, the mixture was diluted with EtOAc and washed with water and brine. The organic layer was dried over sodium sulfate and concentrated to obtain the crude product which was purified by preparative HPLC to obtain 65 mg of racemic compound. ¹H NMR (400 MHz, CDCl₃): δ (ppm): 8.38 (s, 1H), 7.72-7.68 (m, 2H), 7.26-7.13 (m, 6H), 5.41 (s, 2H), 5.24-5.2 (m, 1H), 3.67-3.60 (m, 1H), 3.24-3.06 (m, 3H), 2.55-2.50 (m, 1H), 2.32-2.28 (m, 1H). Separation by chiral HPLC provides Compound Nos. 13a and 13b.

### Example 14: Preparation of Compound No. 14, 14a and 14b

### Step-1: Synthesis of 3-iodo-1-(1,2,3,4-tetrahydronaphthalen-2-yl)-1H-pyrazolo[3,4-d]pyrimidin-4-amine

See Example No. 13.

### Step-2: Synthesis of 3-(3-fluoro-4-methoxyphenyl)-1-(1,2,3,4-tetrahydronaphthalen-2-yl)-1H-pyrazolo[3,4-d]pyrimidin-4-amine

To a suspension of 3-iodo-1-(1,2,3,4-tetrahydronaphthalen-2-yl)-1H-pyrazolo[3,4-d]pyrimidin-4-amine (0.25 g, 0.639 mmol, 1 equiv.) and 3-fluoro-4-methoxyphenylboronic acid (0.162 g, 0.959 mmol, 1.5 equiv.) in DMF:water (2:1) was added sodium carbonate (0.135 g, 1.278 mmol, 2 equiv.) and tetrakis(triphenylphosphine)palladium(0) (73.8 mg, 0.0639 mmol, 0.1 equiv.) at RT in a reagent bottle. The reagent bottle was sealed and the mixture stirred at 100° C. overnight. The reaction was monitored by TLC. After completion of reaction, the mixture was diluted with EtOAc and washed with water and brine. The organic layer was dried over sodium sulfate and concentrated to obtain the crude product which was purified by preparative HPLC to obtain 70 mg of racemic compound 14. 25 mg of this purified compound was then submitted to chiral separation to obtain 3.2 mg peak 1 (Compound No. 14a) and 4.5 mg peak 2 as chiral pure compound (Compound No. 14b). Compound No. 14: ¹H NMR (400 MHz, CDCl₃): δ (ppm): 8.38 (s, 1H), 7.49-7.42 (m, 2H), 7.15-7.099 (m, 5H), 5.47 (s, 2H), 5.25-5.198 (m, 1H), 3.965 (m, 3H), 3.66-3.60 (m, 1H), 3.23-3.03 (m, 3H), 2.54-2.486 (m, 1H), 2.31-2.279 (m, 1H). Compound No. 14a: ¹H NMR (400 MHz, CDCl₃): δ (ppm): 8.38 (s, 1H), 7.49-7.42 (m, 2H), 7.15-7.09 (m, 5H), 5.46 (s, 2H), 5.25-5.193 (m, 1H), 3.964 (m, 3H), 3.66-3.60 (m, 1H), 3.23-3.02 (m, 3H), 2.54-2.50 (m, 1H), 2.31-2.27 (m, 1H). Compound No. 14b: ¹H NMR (400 MHz, CDCl₃): δ (ppm): 8.38 (s, 1H), 7.49-7.42 (m, 2H), 7.15-7.09 (m, 5H), 5.456 (s, 2H), 5.23-5.21 (m, 1H), 3.965 (m, 3H), 3.66-3.60 (m, 1H), 3.23-3.06 (m, 3H), 2.54-2.50 (m, 1H), 2.309-2.28 (m, 1H).

### Example 15: Preparation of Compound Nos. 15, 15a and 15b

### Step-1: Synthesis of 3-iodo-1-(1,2,3,4-tetrahydronaphthalen-2-yl)-1H-pyrazolo[3,4-d]pyrimidin-4-amine

See Example No. 13.

### Step-2: Synthesis of 3-(6-methoxypyridin-3-yl)-1-(1,2,3,4-tetrahydronaphthalen-2-yl)-1H-pyrazolo[3,4-d]pyrimidin-4-amine

To a suspension of 3-iodo-1-(1,2,3,4-tetrahydronaphthalen-2-yl)-1H-pyrazolo[3,4-d]pyrimidin-4-amine (0.25 g, 0.639 mmol, 1 equiv.) and 6-methoxypyridin-3-ylboronic acid (0.146 g, 0.959 mmol, 1.5 equiv.) in DMF:water (2:1) was added sodium carbonate (0.135 g, 1.278 mmol, 2 equiv.) and tetrakis(triphenylphosphine)palladium(0) (73.8 mg, 0.0639 mmol, 0.1 equiv.) at RT in a reagent bottle. The reagent bottle was sealed and the mixture stirred at 100° C. overnight. The reaction was monitored by TLC. After completion of reaction, the mixture was diluted with EtOAc and washed with water and brine. The organic layer was dried over sodium sulfate and concentrated to obtain the crude product which was purified by preparative HPLC to obtain 48 mg of white solid racemic compound. ¹H NMR (400 MHz, CDCl₃): δ (ppm): 8.50 (s, 1H), 8.386 (s, 1H), 7.93 (dd, J=8.4, 2.4, 1H), 7.154-7.096 (m, 4H), 6.91 (d, J=8.4, 1H), 5.531 (s, 2H), 5.26-5.20 (m, 1H), 4.01 (m, 3H), 3.66-3.59 (m, 1H), 3.23-3.06 (m, 3H), 2.54-2.50 (m, 1H), 2.32-2.28 (m, 1H). Separation by chiral HPLC provides Compound Nos. 15a and 15b.

### Example 16: Preparation of Compound Nos. 16, 16a and 16b

### Step-1: Synthesis of 3-iodo-1-tetralin-1-yl-pyrazolo[3,4-d]pyrimidin-4-amine

See Example No. 19.

### Step-2: Synthesis of 3-(1H-pyrrolo[2,3-b]pyridin-5-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (150 mg, 0.383 mmol) and pyrrolo [2,3-b]pyridine-5-boronic acid pinacol ester (140 mg, 0.575 mmol) in DMF (3 mL) was added a solution of sodium carbonate (81 mg, 0.766 mmol) in water (3 mL) followed by the addition of tetrakis(triphenylphosphine)palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated under reduced pressure to obtain a crude product which was purified by reverse phase preparative HPLC to obtain 13 mg of 3-(1H-pyrrolo[2,3-b]pyridin-5-yl)-1-tetralin-2-yl-pyrazolo[3,4-d]pyrimidin-4-amine as a white solid (Racemate). ¹H NMR (400 MHz, DMSO-d₆) δ (ppm): 11.82 (s, 1H), 8.48 (s, 1H), 8.26 (s, 1H), 8.20 (s, 1H), 7.56 (s, 1H), 7.14 (d, J=7.7 Hz, 4H), 6.56 (s, 1H), 5.18-4.99 (m, 1H), 3.25-3.03 (m, 4H), 2.37 (m, 1H), 2.22 (m, 1H). Separation by chiral HPLC provides Compound Nos. 16a and 16b.

### Example 17: Preparation of Compound Nos. 17, 17a and 17b

### Step-1: Synthesis of 3-iodo-1-tetralin-1-yl-pyrazolo[3,4-d]pyrimidin-4-amine

See Example No. 19.

### Step-2: Synthesis of 3-(3,4-dimethoxyphenyl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (150 mg, 0.383 mmol) and 3,4-dimethoxyphenylboronic acid (105 mg, 0.575 mmol) in DMF (3 mL) was added a solution of sodium carbonate (81 mg, 0.766 mmol) in water (3 mL) followed by the addition of tetrakis(triphenylphosphine)palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated under reduced pressure to obtain a crude product which was purified by reverse phase preparative HPLC to obtain 65 mg of 3-(3,4-dimethoxyphenyl)-1-(1,2,3,4-tetrahydronaphthalen-2-yl)-1H-pyrazolo[3,4-d]pyrimidin-4-amine as a white solid (Racemate). ¹H NMR (400 MHz, CDCl₃) δ (ppm): 8.37 (s, 1H), 7.23-7.02 (m, 7H), 5.63 (bs, 2H), 5.22 (td, J=11.4, 5.5 Hz, 1H), 3.95 (s, 6H), 3.67 (dd, J=16.1, 11.3 Hz, 1H), 3.27-2.99 (m, 3H), 2.55 (qd, J=12.1, 5.9 Hz, 1H), 2.35-2.25 (m, 1H). Separation by chiral HPLC provides Compound Nos. 17a and 17b.

### Example 18: Preparation of Compound Nos. 18, 18a and 18b

### Step-1: Synthesis of 3-iodo-1-tetralin-1-yl-pyrazolo[3,4-d]pyrimidin-4-amine

See Example No. 19.

### Step-2: Synthesis of 3-(1H-indol-6-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (150 mg, 0.383 mmol) and 1H-indole-6-boronic acid (93 mg, 0.575 mmol) in DMF (3 mL) was added a solution of sodium carbonate (81 mg, 0.766 mmol) in water (3 mL) followed by the addition of tetrakis(triphenylphosphine)palladium(0) (44 mg, 0.0383 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated under reduced pressure to obtain a crude product which was purified by reverse phase preparative HPLC to obtain 44 mg of 3-(1H-indol-6-yl)-1-(1,2,3,4-tetrahydronaphthalen-2-yl)-1H-pyrazolo[3,4-d]pyrimidin-4-amineas a white solid (Racemate). ¹H NMR (400 MHz, CDCl₃) δ (ppm): 8.57 (d, J=10.5 Hz, 1H), 8.37 (s, 1H), 7.82-7.71 (m, 2H), 7.44 (d, J=8.1 Hz, 1H), 7.30 (s, 1H), 7.17-7.07 (m, 4H), 6.62 (s, 1H), 5.74 (bs, 2H), 5.31-5.18 (m, 1H), 3.69 (dd, J=16.1, 11.4 Hz, 1H), 3.28-2.99 (m, 3H), 2.56 (qd, J=12.1, 5.9 Hz, 1H), 2.32 (m, 1H). Separation by chiral HPLC provides Compound Nos. 18a and 18b.

### Example 19: Preparation of Compound Nos. 19, 19a and 19b

### Step-1: Synthesis of 5-amino-1H-pyrazole-4-carbonitrile

Hydrazine hydrate (15.9 mL, 327.60 mmol) was added dropwise to 2-(ethoxymethylene)propanedinitrile (20 g, 163.80 mmol) under ice-cooled conditions, in a reagent bottle. The reagent bottle was sealed and heated at 100° C. for 1 h. The reaction was monitored by TLC. After completion of reaction, the mixture was cooled to RT and water (50 mL) was added to the reaction mixture. The product was extracted using EtOAc (3×200 mL). The combined organic layer was dried over anhydrous sodium sulfate and concentrated under reduced pressure to obtain 15 g of 5-amino-1H-pyrazole-4-carbonitrile as a light brown solid.

### Step-2: Synthesis of 1H-pyrazolo[3,4-d]pyrimidin-4-amine

To 5-amino-1H-pyrazole-4-carbonitrile (15 g, 138.76 mmol) was added formamide (75 mL) under nitrogen atmosphere and the reaction mixture was heated at 180° C. overnight. The reaction was monitored by TLC. After completion of reaction, the mixture was cooled to 0° C. and water (50 mL) was added, and a precipitate formed. The precipitate was collected by filtration and dried to obtain 13.1 g of 1H-pyrazolo[3,4-d]pyrimidin-4-amine as a yellow solid.

### Step-3: Synthesis of 3-iodo-1H-pyrazolo[3,4-d]pyrimidin-4-amine

To a suspension of 1H-pyrazolo[3,4-d]pyrimidin-4-amine (7.5 g, 55.502 mmol) in anhydrous N,N-dimethyl formamide (50 mL) was added N-iodosuccinimide (49 g, 222.008 mol) portionwise under nitrogen atmosphere. The reaction mixture was heated at 80° C. for 2 h. The reaction was monitored by TLC. After completion of reaction, the mixture was cooled to RT and diluted with EtOAc (400 mL). Saturated aq. sodium thiosulfate solution (100 mL) was added, and a precipitate formed. The precipitate was collected by filtration, washed with additional amount of water, diethyl ether and then dried to obtain 14 g of 3-iodo-1H-pyrazolo[3,4-d]pyrimidin-4-amine as an off-white solid.

### Step-4: Synthesis of 1,2,3,4-tetrahydronaphthalen-2-ol

To a stirred solution of 3, 4-dihydronaphthalen-2(1H)-one (500 mg, 3.420 mmol) in MeOH (5 mL) was added NaBH₄ (259.0 mg, 6.84 mmol) at 0° C. portionwise. The reaction mixture was then stirred at RT for 1 h. The reaction was monitored by TLC. After completion of reaction, the mixture was quenched with ice and concentrated under reduced pressure. The residue obtained was diluted with water (150 mL) and extracted with EtOAc (2×200 mL). The combined organic layer was dried over sodium sulfate and concentrated to afford 500 mg of 1,2,3,4-tetrahydronaphthalen-2-ol as a yellow semi-solid, which was taken to the next step without further purification.

### Step-5: Synthesis of 1,2,3,4-tetrahydronaphthalen-2-yl methanesulfonate

To a stirred solution of 1,2,3,4-tetrahydronaphthalen-2-ol (250 mg, 1.68 mmol) in DCM (5 mL) was added triethylamine (1.17 mL, 8.50 mmol) at 0° C. followed by addition of mesyl chloride (0.26 mL, 3.35 mmol). The reaction mixture was then stirred at 0° C. for 2 h. The reaction was monitored by ¹H NMR. After completion of reaction, the mixture was quenched with ice extracted with DCM (2×50 mL). The combined organic layer was dried over sodium sulfate and concentrated to afford 340 mg of 1,2,3,4-tetrahydronaphthalen-2-yl methanesulfonate as a brown liquid which was taken to the next step without further purification.

### Step-6: Synthesis of 3-iodo-1-(1,2,3,4-tetrahydronaphthalen-2-yl)-1H-pyrazolo[3,4-d]pyrimidin-4-amine

A solution of 3-iodo-1H-pyrazolo[3,4-d]pyrimidin-4-amine (2 g, 7.662 mmol) in 40 mL DMF was cooled to 0° C. To this solution was added sodium hydride (459 mg, 11.493 mmol) under nitrogen atmosphere. The reaction mixture was stirred at the same temperature for 30 min. Then a solution of 1,2,3,4-tetrahydronaphthalen-2-yl methanesulfonate (3.46 g, 15.324 mmol) in 3 mL DMF was added and the reaction mixture was heated at 80° C. for 2 h. The reaction was monitored by TLC. After completion of reaction, the mixture was cooled to 0° C. and quenched by ice cold water. The product was extracted using EtOAc (2×100 mL). The combined organic layer was again washed with water (3×50 mL), and then with brine (50 mL). The organic layer was separated, dried over anhydrous sodium sulfate and concentrated under reduced pressure to obtain a crude product. The crude product was purified by precipitating in DCM-pentane, filtered, washed with diethyl ether and dried to obtain 1.1 g of 3-iodo-1-tetralin-2-yl-pyrazolo[3,4-d]pyrimidin-4-amine as an off-white solid.

### Step-7: Synthesis of 3-(1H-benzo[d]imidazol-5-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (300 mg, 0.766 mmol) and 1H-benzimidazole-5-boronic acid pinacol ester (280 mg, 1.150 mmol) in DMF (3 mL) was added a solution of sodium carbonate (162 mg, 1.532 mmol) in water (3 mL) followed by the addition of tetrakis(triphenylphosphine)palladium(0) (89 mg, 0.0766 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×25 mL) and brine (25 mL). The organic layer was separated, dried over anhydrous sodium sulfate and concentrated to obtain a crude product which was purified by reverse phase preparative HPLC to obtain 70 mg of 3-(1H-benzimidazol-5-yl)-1-tetralin-2-yl-pyrazolo[3,4-d]pyrimidin-4-amine as a white solid. The pair of enantiomers was again separated using chiral HPLC to obtain 8 mg each of peak 1 (Compound No. 19a) and peak 2 (Compound No. 19b) as chirally pure compounds. Compound No. 19a: ¹H NMR (400 MHz, CDCl₃) δ (ppm): 9.53-9.46 (bs, 1H), 8.40 (s, 1H), 8.15 (s, 1H), 7.99 (d, J=8.4 Hz, 1H), 7.87 (s, 1H), 7.67 (dt, J=18.1, 8.7 Hz, 1H), 7.18-7.07 (m, 4H), 5.50 (s, 2H), 5.26 (ddd, J=16.0, 11.5, 4.6 Hz, 1H), 3.67 (dd, J=16.1, 11.6 Hz, 1H), 3.29-3.00 (m, 3H), 2.56 (qd, J=12.2, 6.1 Hz, 1H), 2.33 (dd, J=12.5, 4.4 Hz, 1H). Compound No. 19b: ¹H NMR (400 MHz, CDCl₃) δ (ppm): 9.44 (d, J=19.1 Hz, 1H), 8.40 (s, 1H), 8.16 (s, 1H), 8.00 (d, J=8.3 Hz, 1H), 7.88 (s, 1H), 7.67 (dt, J=18.9, 8.7 Hz, 1H), 7.18-7.07 (m, 4H), 5.49 (s, 2H), 5.26 (ddd, J=16.2, 11.5, 4.7 Hz, 1H), 3.68 (ddd, J=15.7, 11.3, 3.9 Hz, 1H), 3.30-3.01 (m, 3H), 2.56 (qt, J=11.5, 4.7 Hz, 1H), 2.38-2.29 (m, 1H). Separation by chiral HPLC provides Compound Nos. 19a and 19b.

### Example 20: Preparation of Compound Nos. 20, 20a and 20b

### Step-1: Synthesis of 3-iodo-1-tetralin-1-yl-pyrazolo[3,4-d]pyrimidin-4-amine

See Example No. 19.

### Step-2: Synthesis of 3-(1H-pyrazol-4-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (50 mg, 0.127 mmol) and 1H-pyrazole-4-boronic acid pinacol ester (37 mg, 0.191 mmol) in DMF (2 mL) was added a solution of sodium carbonate (27 mg, 0.254 mmol) in water (2 mL) followed by the addition of tetrakis(triphenylphosphine) palladium(0) (15 mg, 0.0127 mmol). The reaction mixture was heated in a reagent bottle at 100° C. overnight. The reaction was monitored by TLC. After completion of reaction, water (20 mL) was added to the reaction mixture and the product was extracted using EtOAc (2×20 mL). The combined organic layer was again washed with water (2×20 mL) and brine (15 mL). The organic layer was separated, dried over anhydrous sodium sulfate and concentrated to obtain a crude product which was purified by reverse phase preparative HPLC to obtain 5 mg of 3-(1H-pyrazol-4-yl)-1-(1,2,3,4-tetrahydronaphthalen-2-yl)-1H-pyrazolo[3,4-d]pyrimidin-4-amine as a white solid (Racemate). ¹H NMR (400 MHz, DMSO-d₆) δ (ppm): 13.21 (bs, 1H), 8.22 (s, 1H), 8.12 (s, 1H), 7.82 (s, 1H), 7.14 (d, J=9.8 Hz, 4H), 5.10-4.99 (m, 1H), 3.49-2.91 (m, 4H), 2.40-2.25 (m, 1H), 2.16 (d, J=12.8 Hz, 1H). Separation by chiral HPLC provides Compound Nos. 20a and 20b.

### Example 21: Preparation of Compound Nos. 21, 21a and 21b

### Step-1: Synthesis of 3-iodo-1-tetralin-1-yl-pyrazolo[3,4-d]pyrimidin-4-amine

See Example No. 19.

### Step-2: Synthesis of 4-(4-amino-1-(1,2,3,4-tetrahydronaphthalen-2-yl)-1H-pyrazolo[3,4-d]pyrimidin-3-yl)phenol

To a solution of 3-iodo-1-tetralin-2-yl-pyrazolo[3,4-d]pyrimidin-4-amine (200 mg, 0.511 mmol) and 4-hydroxyphenylboronic acid pinacol ester (168 mg, 0.766 mmol) in DMF (3 mL) was added a solution of sodium carbonate (108 mg, 1.022 mmol) in water (2 mL) followed by the addition of tetrakis(triphenylphosphine)palladium(0) (59 mg, 0.0511 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated to obtain a crude product which was purified by reverse phase preparative HPLC to obtain 55 mg of 4-[4-amino-1-(1,2,3,4-tetrahydronaphthalen-2-yl)-1H-pyrazolo[3,4-d]pyrimidin-3-yl]phenol as an off-white solid (Racemate). ¹H NMR (400 MHz, DMSO-d₆) δ (ppm): 9.76 (s, 1H), 8.23 (s, 1H), 7.48 (d, J=8.3 Hz, 2H), 7.20-7.10 (m, 4H), 6.92 (d, J=8.3 Hz, 2H), 5.06 (dq, J=11.2, 5.5, 4.3 Hz, 1H), 3.43 (dd, J=16.3, 11.0 Hz, 1H), 3.28-2.91 (m, 3H), 2.34 (ddt, J=17.6, 11.7, 5.7 Hz, 1H), 2.23-2.14 (m, 1H). Separation by chiral HPLC provides Compound Nos. 21a and 21b.

### Example 22: Preparation of Compound Nos. 22, 22a and 22b

### Step-1: Synthesis of 3-iodo-1-(1,2,3,4-tetrahydronaphthalen-2-yl)-1H-pyrazolo[3,4-d]pyrimidin-4-amine

See Example No. 19.

### Step-2: Synthesis of 3-(3-fluoro-4-methoxyphenyl)-1-(1,2,3,4-tetrahydronaphthalen-2-yl)-1H-pyrazolo[3,4-d]pyrimidin-4-amine

To a suspension of 3-iodo-1-(1,2,3,4-tetrahydronaphthalen-2-yl)-1H-pyrazolo[3,4-d]pyrimidin-4-amine (0.15 g, 0.38 mmol, 1 equiv.) and 3-fluoro-4-methoxyphenylboronic acid (0.097 g, 0.57 mmol, 1.5 equiv.) in DMF:water (2:1) was added sodium carbonate (0.08 g, 0.76 mmol, 2 equiv.) and tetrakis(triphenylphosphine)palladium(0) (44 mg, 0.038 mmol, 0.1 equiv.) at RT in a reagent bottle. The reagent bottle was sealed and the mixture stirred at 100° C. overnight. The reaction was monitored by TLC. After completion of reaction, the mixture was diluted with EtOAc and washed with water and brine. The organic layer was dried over sodium sulfate and concentrated to obtain 170 mg of crude product.

### Step-3: Synthesis of 4-(4-amino-1-(1,2,3,4-tetrahydronaphthalen-2-yl)-1H-pyrazolo[3,4-d]pyrimidin-3-yl)-2-fluorophenol

To a suspension of 3-(3-fluoro-4-methoxyphenyl)-1-(1,2,3,4-tetrahydronaphthalen-2-yl)-1H-pyrazolo[3,4-d]pyrimidin-4-amine (0.15 g, 0.385 mmol) in 5 mL DCM was added 1.92 mL of BBr₃ (1M solution in DCM, 1.92 mmol, 5 equiv.). The reaction was allowed to stir at RT overnight. The reaction was monitored by LCMS. After completion of reaction, the mixture was concentrated to obtain the crude product which was purified by preparative HPLC to afford 3.5 mg of off white solid as the free base. ¹H NMR (400 MHz, DMSO): δ (ppm): 10.16 (bs, 1H), 8.23 (s, 1H), 7.38 (dd, J=11.8, 1.6, 1H), 7.297 (d, J=8, 1H), 7.15-7.07 (m, 7H), 5.08-5.05 (m, 1H), 3.46-2.99 (m, 4H), 2.36-2.17 (m, 2H). Separation by chiral HPLC provides Compound Nos. 22a and 22b.

### Example 23: Preparation of Compound Nos. 23, 23a and 23b

### Step-1: Synthesis of 3-iodo-1-tetralin-2-yl-pyrazolo[3,4-d]pyrimidin-4-amine

See Example No. 19.

### Step-2: Synthesis of 3-fluoro-2-methoxy-5-(4,4,5,5-tetramethyl-1,3,2-dioxaborolan-2-yl)pyridine

Nitrogen gas was purged through a mixture of 5-chloro-3-fluoro-2-methoxy-pyridine (700 mg, 4.331 mmol), bis(pinacolato)diboron (1.32 g, 5.198 mmol) and potassium acetate (1.27 g, 12.993 mmol) in 1,4-dioxane (10 mL) for 30 min. Then, [1,1-bis-(diphenylphosphino)-ferrocene]palladium(II)chloride complex with DCM (353 mg, 0.433 mmol) was added. Nitrogen gas purging was continued for another 5 min. The reaction mixture was heated at 100° C. overnight. The reaction was monitored by LCMS. After completion of reaction, the mixture was cooled to RT, then filtered through a celite bed. The organic solvent was removed under reduced pressure and the residue obtained was triturated with pentane. The pentane layer was taken and concentrated to obtain 1.01 g of 3-fluoro-2-methoxypyridine-5-boronic acid pinacol ester which was used as such for next step without further purification.

### Step-3: Synthesis of 3-(5-fluoro-6-methoxypyridin-3-yl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (200 mg, 0.511 mmol) and 3-fluoro-2-methoxypyridine-5-boronic acid pinacol ester (194 mg, 0.766 mmol) in DMF (3 mL) was added a solution of sodium carbonate (108 mg, 1.022 mmol) in water (3 mL) followed by the addition of tetrakis(triphenylphosphine) palladium(0) (59 mg, 0.0511 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×20 mL) and brine (20 mL). The organic layer was separated, dried over anhydrous sodium sulfate and concentrated to obtain a crude product that was purified by reverse phase preparative HPLC to obtain 33 mg of 3-(5-fluoro-6-methoxypyridin-3-yl)-1-(1,2,3,4-tetrahydronaphthalen-2-yl)-1H-pyrazolo[3,4-d]pyrimidin-4-amine as the TFA salt (white solid). ¹H NMR (400 MHz, CD₃OD) δ (ppm): 8.36 (s, 1H), 8.25 (s, 1H), 7.81 (d, J=10.5 Hz, 1H), 7.18-7.07 (m, 4H), 5.24 (td, J=11.0, 5.4 Hz, 1H), 4.09 (s, 3H), 3.54 (dd, J=16.1, 10.9 Hz, 1H), 3.26-2.99 (m, 3H), 2.48 (qd, J=11.7, 6.5 Hz, 1H), 2.35-2.23 (m, 1H). Separation by chiral HPLC provides Compound Nos. 23a and 23b.

### Example 24: Preparation of Compound Nos. 24, 24a and 24b

### Step-1: Synthesis of 1H-pyrazolo[3,4-d]pyrimidin-4-amine

See Example No. 19.

### Step-2: Synthesis of 3-(6-methoxypyridin-3-yl)-1-(1,2,3,4-tetrahydronaphthalen-2-yl)-1H-pyrazolo[3,4-d]pyrimidin-4-amine

To a suspension of 3-iodo-1-(1,2,3,4-tetrahydronaphthalen-2-yl)-1H-pyrazolo[3,4-d]pyrimidin-4-amine (0.25 g, 0.639 mmol, 1 equiv.) and 6-methoxypyridin-3-ylboronic acid (0.146 g, 0.959 mmol, 1.5 equiv.) in DMF:water (2:1) was added sodium carbonate (0.135 g, 1.278 mmol, 2 equiv.) and tetrakis(triphenylphosphine)palladium(0) (73.8 mg, 0.0639 mmol, 0.1 equiv.) at RT in a reagent bottle. The reagent bottle was sealed and stirred at 100° C. overnight. The reaction was monitored by TLC. After completion of reaction, the mixture was diluted with EtOAc and washed with water and brine. The organic layer was dried over sodium sulfate and concentrated to obtain the crude product that was purified by preparative HPLC to obtain 48 mg of white solid racemic product.

### Step-3: Synthesis of 5-(4-amino-1-(1,2,3,4-tetrahydronaphthalen-2-yl)-1H-pyrazolo[3,4-d]pyrimidin-3-yl)pyridin-2-ol

To a solution of the 3-(6-methoxypyridin-3-yl)-1-(1,2,3,4-tetrahydronaphthalen-2-yl)-1H-pyrazolo[3,4-d]pyrimidin-4-amine from step 2, in 5 mL DCM was added 0.1 mL TMS iodide at RT. The reaction was monitored by LCMS. After completion of reaction, the mixture was concentrated and the residues purified by HPLC to give the desired product. ¹H NMR (400 MHz, DMSO): δ (ppm): 12.2-11.8 (broad S, 1H), 8.325 (s, 1H), 7.70-7.607 (m, 2H), 7.23-6.97 (m, 6H), 6.47 (d, J=9.2, 1H), 5.08-5.07 (m, 1H), 3.16-2.97 (m, 4H), 2.34-2.17 (m, 2H). Separation by chiral HPLC provides Compound Nos. 24a and 24b.

### Example 25: Preparation of Compound Nos. 25, 25a and 25b

### Step-1: Synthesis of 3-iodo-1-tetralin-2-yl-pyrazolo[3,4-d]pyrimidin-4-amine

See Example No. 19.

### Step-2: Synthesis of 3-(6-chloro-3-pyridyl)-1-tetralin-2-yl-pyrazolo[3,4-d]pyrimidin-4-amine

To a solution of 3-iodo-1-tetralin-2-yl-pyrazolo[3,4-d]pyrimidin-4-amine (150 mg, 0.383 mmol) and (6-chloro-3-pyridyl)boronic acid (90 mg, 0.575 mmol) in DMF (4 mL) was added a solution of sodium carbonate (81 mg, 0.766 mmol) in water (4 mL) followed by the addition of tetrakis(triphenylphosphine) palladium(0) (44 mg, 0.038 mmol). The resultant reaction mixture was heated in a reagent bottle at 100° C. overnight. The progress of reaction was monitored by LCMS. After completion of reaction, water (10 mL) was added to the reaction mixture and the product was extracted using EtOAc (2×30 mL). The combined organic layer was again washed with water (2×25 mL) and brine (25 mL). The organic layers were separated, dried over anhydrous sodium sulfate and concentrated to obtain a crude product. The crude product was purified by reverse phase preparative HPLC to obtain 3-(6-chloro-3-pyridyl)-1-tetralin-2-yl-pyrazolo[3,4-d]pyrimidin-4-amine as the TFA salt (17.05 mg), a white solid. LCMS: 376.84 (M+1). ¹H NMR (400 MHz, CD₃OD, TFA salt) δ (ppm): 8.69 (d, J=2.4 Hz, 1H), 8.35 (s, 1H), 8.14 (dd, J=8.3, 2.5 Hz, 1H), 7.64 (d, J=8.2 Hz, 1H), 7.13 (m, 4H), 5.25 (m, 1H), 3.54 (dd, J=15.9, 11.2 Hz, 1H), 3.26-2.9 (m, 3H), 2.47 (m, 1H), 2.30 (m, 1H). Separation by chiral HPLC provides Compound Nos. 25a and 25b.

### Example 26: Preparation of Compound Nos. 26, 26a and 26b

### Step-1: Synthesis of 3-iodo-1-tetralin-2-yl-pyrazolo[3,4-d]pyrimidin-4-amine

See Example No. 19.

### Step-2: Synthesis of 3-(4-fluoro-3-methoxyphenyl)-1-(1,2,3,4-tetrahydronaphthalen-2-yl)-1H-pyrazolo[3,4-d]pyrimidin-4-amine

To a solution of 3-iodo-1-tetralin-2-yl-pyrazolo[3,4-d]pyrimidin-4-amine (300 mg, 0.766 mmol) and 4-fluoro-3-methoxyphenylboronic acid (195 mg, 1.150 mmol) in DMF (3 mL) was added a solution of sodium carbonate (162 mg, 1.532 mmol) in water (3 mL) followed by the addition of tetrakis(triphenyl-phosphine)palladium(0) (89 mg, 0.0766 mmol). The reaction mixture was heated in a reagent bottle at 100° C. overnight. The reaction was monitored by TLC. After completion of reaction, water (20 mL) was added to the reaction mixture and the product was extracted using EtOAc (2×30 mL). The combined organic layer was again washed with water (2×25 mL) and brine (25 mL). The organic layer was separated, dried over anhydrous sodium sulfate and concentrated to obtain 300 mg of 3-(4-fluoro-3-methoxy-phenyl)-1-tetralin-2-yl-pyrazolo[3,4-d]pyrimidin-4-amine as a crude product which was used as such without further purification.

### Step-3: Synthesis of 5-(4-amino-1-(1,2,3,4-tetrahydronaphthalen-2-yl)-1H-pyrazolo[3,4-d]pyrimidin-3-yl)-2-fluorophenol

To a stirred solution of 3-(4-fluoro-3-methoxy-phenyl)-1-tetralin-2-yl-pyrazolo[3,4-d]pyrimidin-4-amine (300 mg, 0.770 mmol) in DCM (10 mL) was added dropwise BBr₃ (3.8 mL, 3.80 mmol, 1 M solution in DCM) at RT under nitrogen atmosphere. The reaction mixture was stirred at RT for 3 h. The reaction was monitored by TLC. After completion of reaction, the mixture was concentrated under reduced pressure. To the residue was added satd. aq. sodium bicarbonate solution (20 mL) and the mixture was extracted using EtOAc (50 mL). The organic layer was again washed with water (20 mL) and finally with brine (20 mL). The organic layer was separated, dried over anhydrous sodium sulfate and concentrated under reduced pressure to afford the crude product which was purified using reverse phase preparative HPLC to get 70 mg of 5-[4-amino-1-(1,2,3,4-tetrahydronaphthalen-2-yl)-1H-pyrazolo[3,4-d]pyrimidin-3-yl]-2-fluorophenol as a white solid (Racemate). The pair of enantiomers was separated using chiral HPLC to obtain 11 mg of compound no. 26a (peak 1) (white solid) and 12 mg of compound no. 26b (peak 2) (white solid) as chirally pure compounds. Compound No. 26: ¹H NMR (400 MHz, CDCl₃) δ (ppm): 8.23 (s, 1H), 7.32-7.06 (m, 7H), 5.06 (td, J=11.1, 5.3 Hz, 1H), 3.22-2.97 (m, 4H), 2.31 (m, 1H), 2.18 (m, 1H). Compound No. 26a: ¹H NMR (400 MHz, CDCl₃) δ (ppm): 8.39 (s, 1H), 7.37-7.07 (m, 7H), 5.46 (bs, 2H), 5.23 (ddt, J=15.3, 11.4, 4 Hz, 1H), 3.63 (dd, J=16.1, 11.3 Hz, 1H), 3.26-3.00 (m, 3H), 2.52 (qd, J=12.2, 6 Hz, 1H), 2.30 (m, 1H). Compound No. 26b: ¹H NMR (400 MHz, CDCl₃) δ (ppm): 8.39 (s, 1H), 7.37-7.04 (m, 7H), 5.48 (bs, 2H), 5.22 (ddt, J=11.7, 8.8, 4.2 Hz, 1H), 3.63 (dd, J=16.1, 11.4 Hz, 1H), 3.26-2.99 (m, 3H), 2.52 (qd, J=12.1, 5.9 Hz, 1H), 2.35-2.25 (m, 1H).

### Example 27: Preparation of Compound Nos. 27, 27a and 27b

### Step-1: Synthesis of 3-iodo-1-tetralin-2-yl-pyrazolo[3,4-d]pyrimidin-4-amine

See Example No. 19.

### Step-2: Synthesis of 3-(4-amino-1-tetralin-2-yl-pyrazolo[3,4-d]pyrimidin-3-yl)benzamide

To a solution of 3-iodo-1-tetralin-2-yl-pyrazolo[3,4-d]pyrimidin-4-amine (150 mg, 0.383 mmol) and (3-carbamoylphenyl)boronic acid (94 mg, 0.575 mmol) in DMF (4 mL) was added a solution of sodium carbonate (81 mg, 0.766 mmol) in water (4 mL) followed by the addition of tetrakis(triphenylphosphine) palladium(0) (44 mg, 0.038 mmol). The resultant reaction mixture was heated in a reagent bottle at 100° C. overnight. The progress of the reaction was monitored by LCMS. After completion of reaction, water (10 mL) was added to the reaction mixture and the product was extracted using EtOAc (2×30 mL). The combined organic layers were washed with water (2×25 mL) and brine (25 mL). The organic layer was separated, dried over anhydrous sodium sulfate and concentrated to obtain a crude product. The crude product was purified by reverse phase preparative HPLC to obtain 3-(4-amino-1-tetralin-2-yl-pyrazolo[3,4-d]pyrimidin-3-yl)benzamide as the TFA salt (24.75 mg), a white solid. LCMS: 384.43 (M+1). ¹H NMR (400 MHz, CD₃OD, TFA salt) δ (ppm): 8.39 (s, 1H), 8.24 (s, 1H), 8.04 (d, J=8.00 Hz, 1H) 7.91 (d, J=7.6, 1H), 7.69 (t, J=7.8 Hz, 1H), 7.22-7.07 (m, 4H), 5.28 (m, 1H), 3.57 (dd, J=16.0, 11.1 Hz, 1H), 3.22-3.03 (m, 3H), 2.50 (m, 1H), 2.31 (m, 1H). Separation by chiral HPLC provides Compound Nos. 27a and 27b.

### Example 28: Preparation of Compound Nos. 28, 28a and 28b

### Step-1: Synthesis of 3-iodo-1-tetralin-2-yl-pyrazolo[3,4-d]pyrimidin-4-amine

See Example No. 19.

### Step-2: Synthesis of 4-(4-amino-1-tetralin-2-yl-pyrazolo[3,4-d]pyrimidin-3-yl)benzamide

To a solution of 3-iodo-1-tetralin-2-yl-pyrazolo[3,4-d]pyrimidin-4-amine (150 mg, 0.383 mmol) and (4-carbamoylphenyl)boronic acid (94 mg, 0.575 mmol) in DMF (4 mL) was added a solution of sodium carbonate (81 mg, 0.766 mmol) in water (4 mL) followed by the addition of tetrakis(triphenylphosphine)palladium(0) (44 mg, 0.038 mmol). The resultant reaction mixture was heated in a reagent bottle at 100° C. overnight. The progress of the reaction was monitored by LCMS. After completion of reaction, water (10 mL) was added to the reaction mixture and the product was extracted using EtOAc (2×30 mL). The combined organic layers were washed with water (2×25 mL) and brine (25 mL). The organic layer was separated, dried over anhydrous sodium sulfate and concentrated to obtain a crude product. The crude product was purified by reverse phase preparative HPLC to obtain 4-(4-amino-1-tetralin-2-yl-pyrazolo[3,4-d]pyrimidin-3-yl)benzamide as the TFA salt (40.34 mg), a white solid. LCMS: 384.43 (M+1). ¹H NMR (400 MHz, CD₃OD, TFA salt) δ (ppm): 8.38 (s, 1H), 8.12-7.97 (m, 2H), 7.90-7.71 (m, 2H), 7.14 (m, 4H), 5.26 (m, 1H), 3.56 (dd, J=16.1, 11.1 Hz, 1H), 3.27-3.09 (m, 3H), 2.50 (m, 1H), 2.38-2.21 (m, 1H). Separation by chiral HPLC provides Compound Nos. 28a and 28b.

### Example 29: Preparation of Compound No. 29

### Step-1: Synthesis of 1-indan-2-yl-3-iodo-pyrazolo[3,4-d]pyrimidin-4-amine

See Example No. 19.

### Step-2: Synthesis of 3-(4-fluoro-3-methoxy-phenyl)-1-indan-2-yl-pyrazolo[3,4-d]pyrimidin-4-amine

To a solution of 1-indan-2-yl-3-iodo-pyrazolo[3,4-d]pyrimidin-4-amine (200 mg, 0.530 mmol) and (4-fluoro-3-methoxy-phenyl)boronic acid (135 mg, 0.795 mmol) in DMF (3 mL) was added a solution of sodium carbonate (112 mg, 1.060 mmol) in water (3 mL) followed by the addition of tetrakis(triphenylphosphine)palladium(0) (61 mg, 0.053 mmol). The reaction mixture was heated in a reagent bottle at 100° C. overnight. The reaction was monitored by TLC. After completion of reaction, water (20 mL) was added to the reaction mixture and the product was extracted with EtOAc (2×20 mL). The combined organic layer was again washed with water (2×15 mL) and finally with brine solution (15 mL). The organic layer was separated, dried over anhydrous sodium sulfate and concentrated under reduced pressure to afford 3-(4-fluoro-3-methoxy-phenyl)-1-indan-2-yl-pyrazolo[3,4-d]pyrimidin-4-amine (190 mg) as a light brown solid, which was used as such for the next step without further purification.

### Step-3: Synthesis of 5-(4-amino-1-indan-2-yl-pyrazolo[3,4-d]pyrimidin-3-yl)-2-fluoro-phenol

To a stirred suspension of 3-(4-fluoro-3-methoxy-phenyl)-1-indan-2-yl-pyrazolo[3,4-d]pyrimidin-4-amine (190 mg, 0.506 mmol) in DCM (10 mL) was added a 1M solution of boron tribromide in DCM (2.5 mL, 2.530 mmol) at RT under nitrogen atmosphere. The reaction mixture was stirred at RT for 3h. The reaction was monitored by LCMS. After completion of reaction, the mixture was concentrated under reduced pressure. To the residue was added saturated solution sodium bicarbonate (20 mL) and the product was extracted with EtOAc (50 mL). The organic layer was again washed with water (20 mL) and finally with brine solution (20 mL). The organic layer was separated, dried over anhydrous sodium sulfate and concentrated under reduced pressure to afford the crude product which was purified using reverse phase preparative HPLC to afford 5-(4-amino-1-indan-2-yl-pyrazolo[3,4-d]pyrimidin-3-yl)-2-fluoro-phenol (76 mg) as a white solid. LCMS: 362.0 (M+1). ¹H NMR (400 MHz, DMSO-d6) δ (ppm): 8.26 (s, 1H), 7.27-7.12 (m, 6H), 7.03 (ddd, J=8.4, 4.3, 2.2 Hz, 1H), 5.72 (p, J=7.9 Hz, 1H), 3.47 (d, J=7.9 Hz, 4H).

### Example 30: Preparation of Compound Nos. 30, 30a and 30b

### Step-1: Synthesis of 5-amino-1H-pyrazole-4-carbonitrile

Ethoxymethylenemalonitrile (20 g, 163.8 mmol) was charged in a flask at 0° C. and hydrazine hydrate (16 mL, 327.6 mmol) was added dropwise. After completion of addition, the reaction mixture was heated at 100° C. for 0.5 h. Ice cold water (10 mL) was added and the reaction mixture was extracted in EtOAc (5×100 mL). The reaction mixture was dried over anhydrous sodium sulfate and concentrated to get 10.9 g of 5-amino-1H-pyrazole-4-carbonitrile.

### Step-2: Synthesis of 1H-pyrazolo[3,4-d]pyrimidin-4-amine

5-Amino-1H-pyrazole-4-carbonitrile (10.9 g, 100.9 mmol) and formamide (40 mL, 10 fold) was charged in a flask and the reaction mixture was heated at 180° C. overnight. Ice cold water (10 mL) was added and the solid obtained was filtered and dried under vacuum to get 7.6 g of 1H-pyrazolo[3,4-d]pyrimidin-4-amine.

### Step-3: Synthesis of 3-iodo-1H-pyrazolo[3,4-d]pyrimidin-4-amine

1H-Pyrazolo[3,4-d]pyrimidin-4-amine (2 g, 14.8 mmol) was charged in DMF (12 mL) and added N-iodosuccinimide (6.3 g, 28.6 mmol). The reaction mixture was heated at 100° C. for 4 h. The solid obtained was filtered off and washed with cold EtOH (15 mL). The solid obtained was dried under vacuum to get (4 g) 3-iodo-1H-pyrazolo[3,4-d]pyrimidin-4-amine.

### Step-4: Synthesis of 3-(3-fluoro-5-methoxyphenyl)-1H-pyrazolo[3,4-d]pyrimidin-4-amine

3-Iodo-1H-pyrazolo [3,4-d]pyrimidin-4-amine (3 g, 11.45 mmol), tetrakistriphenylphosphinepalladium (0) (1.3 g, 1.145 mmol) was charged in DME (60 mL) and the mixture purged with nitrogen for 5 min. Potassium carbonate (1.5 g, 11.45 mmol) and 3-fluoro-5-methoxyphenylboronic acid (5.4 g, 34.55 mmol) was added. Water (60 mL) was added and again purged with nitrogen for 5 min. The reaction mixture was stirred at 100° C. for 24 h. The reaction was monitored by TLC and LCMS. Then reaction mixture was acidified with 2M HCl and the aqueous layer was separated with EtOAc (3×100 mL). The aqueous layer was basified with saturated sodium carbonate solution and the solid obtained was filtered off. The solid obtained was washed with water (10 mL) and dried under vacuum to get 1.3 g of 3-(3-fluoro-5-methoxyphenyl)-1H-pyrazolo[3,4-d]pyrimidin-4-amine.

### Step-5: Synthesis of indan-1-yl methanesulfonate

Indan-1-ol (200 mg, 1.49 mmol) was charged in DCM (15 mL). Triethylamine (1 mL, 7.45 mmol) was added and the reaction mixture was cooled to 0° C. Mesyl chloride (0.25 mL, 2.98 mmol) was added dropwise and the reaction mixture was stirred at the same temperature for 30 min. The reaction was monitored by TLC and ice water (5 mL) was added. The mixture was extracted with EtOAc (2×25 mL). The combined organic layers were washed with water (3×25 mL), dried over anhydrous sodium sulfate and concentrated under reduced pressure to get 195 mg of indan-1-yl methanesulfonate.

### Step-6: Synthesis of 3-(3-fluoro-5-methoxy-phenyl)-1-indan-1-yl-pyrazolo[3,4-d]pyrimidin-4-amine

3-(3-Fluoro-5-methoxyphenyl)-1H-pyrazolo[3,4-d]pyrimidin-4-amine (25 mg, 0.096 mmol) was charged in DMF (1 mL). The reaction mixture was cooled to 0° C. and sodium hydride (11.5 mg, 0.288 mmol) was added. After 10 min, indan-1-yl methanesulfonate (41 mg, 0.192 mmol) was added dropwise. The reaction mixture was allowed to come to RT and stirred at RT overnight. The reaction was monitored by TLC and LCMS and allowed to come to RT. Water (10 mL) was added and the mixture was extracted with EtOAc (3×25 mL). The combined organic layers were washed with water (3×25 mL) and dried over anhydrous sodium sulfate. The solvent was evaporated under reduced pressure to get 21 mg of 3-(3-fluoro-5-methoxy-phenyl)-1-indan-1-yl-pyrazolo[3,4-d]pyrimidin-4-amine.

### Step-7: Synthesis of 3-(4-amino-1-indan-1-yl-pyrazolo[3,4-d]pyrimidin-3-yl)-5-fluoro-phenol

3-(3-Fluoro-5-methoxy-phenyl)-1-indan-1-yl-pyrazolo[3,4-d]pyrimidin-4-amine (20 mg, 0.053 mmol) was charged in DCM (1 mL) and the reaction mixture was cooled to 0° C. A 1M solution of boron tribromide (0.16 mL, 159 mmol) was added dropwise. The reaction mixture was allowed to come to RT and stirred at RT for 4 h. The reaction was monitored by TLC and LCMS. Saturated sodium bicarbonate was added and the mixture extracted with EtOAc (4×25 mL). The combined organic layer was dried over anhydrous sodium sulfate and concentrated under reduced pressure and purified using reverse phase HPLC to get 1.5 mg of 3-(4-amino-1-indan-1-yl-pyrazolo[3,4-d]pyrimidin-3-yl)-5-fluoro-phenol. ¹H NMR (400 MHz, CD₃OD) δ (ppm): 8.43-8.33 (bs, 1H), 7.30-7.15 (m, 4H), 6.94-6.83 (m, 2H), 6.66 (dt, J=10.6, 2.4 Hz, 1H), 5.81 (p, J=8.1 Hz, 1H), 3.61 (dd, J=15.9 Hz, 7.8 HZ, 2H), 3.49 (dd, J=15.8 Hz, 8.4 Hz, 2H). Separation by chiral HPLC provides Compound Nos. 30a and 30b.

### Example 31: Preparation of Compound Nos. 31, 31a and 31b

### Step-1: Synthesis of 3-iodo-1H-pyrazolo[3,4-d]pyrimidin-4-amine

See Example No. 19.

### Step-2: Synthesis of tert-butyl 3-(4-amino-3-iodo-1H-pyrazolo[3,4-d]pyrimidin-1-yl)-6-methyl-3,4-dihydroquinoline-1(2H)-carboxylate

To a stirred suspension of 3-iodo-1H-pyrazolo[3,4-d]pyrimidin-4-amine (1 g, 3.831 mmol) in DMF (20 mL) was added Cs₂CO₃ (3.744 g, 11.49 mmol) at RT and the reaction mixture was allowed to stir for 15 min. Then, tetrabutylammonium iodide (0.682 g, 1.84 mmol) and tert-butyl 6-methyl-3-(methylsulfonyloxy)-3,4-dihydroquinoline-1(2H)-carboxylate (2.41 g, 7.66 mmol) were successively added and the mixture was heated at 65° C. for 90 min. The reaction was monitored by TLC. After completion of reaction, the mixture was cooled to RT and diluted with water (150 mL) and extracted with EtOAc (3×200 mL). The combined organic layers were washed with water (2×400 mL), dried over sodium sulfate and concentrated. The crude product was purified by flash chromatography using 5% MeOH/DCM. The compound obtained was triturated with diethyl ether/hexane (1:1) to afford 400 mg of tert-butyl 3-(4-amino-3-iodo-1H-pyrazolo[3,4-d]pyrimidin-1-yl)-6-methyl-3,4-dihydroquinoline-1(2H)-carboxylate as a white solid.

### Step-3: Synthesis of 3-iodo-1-(6-methyl-1,2,3,4-tetrahydroquinolin-3-yl)-1H-pyrazolo[3,4-d]pyrimidin-4-amine

TFA (4 mL) was added dropwise at 0° C. to a solution of tert-butyl 3-(4-amino-3-iodo-1H-pyrazolo[3,4-d]pyrimidin-1-yl)-6-methyl-3,4-dihydroquinoline-1(2H)-carboxylate (400 mg, 0.790 mmol) in DCM (12 mL) and the resultant reaction mixture was stirred at 90° C. for 90 min. The reaction was monitored by TLC. After completion of reaction, the mixture was concentrated under reduced pressure. The residue obtained was washed with diethyl ether/hexane (1:1) to afford 500 mg of 3-iodo-1-(6-methyl-1,2,3,4-tetrahydroquinolin-3-yl)-1H-pyrazolo[3,4-d]pyrimidin-4-amine as the TFA salt.

### Step-4: Synthesis of (3-fluoro-5-hydroxyphenyl)boronic acid

To a stirred solution of 3-fluoro-5-methoxyphenylboronic acid (400 mg, 2.35 mmol) in DCM (10 mL) was added BBr₃ (1M solution in DCM; 3.53 mL, 3.53 mmol) at 0° C. dropwise and the resultant reaction mixture was gradually warmed to RT and stirred for 16 h. The reaction was monitored by TLC and through ¹H NMR. After 16 h, the reaction mixture was slowly quenched using 3N-HCl at ice-cold condition and then extracted with EtOAc (22×50 mL). The combined organic layers were washed with brine (30 mL), dried over sodium sulfate and concentrated. The crude compound was triturated with pentane (2×20 mL) to afford 260 mg of 3-fluoro-5-hydroxyphenylboronic acid as an off-white solid.

### Step-5: Synthesis of 3-(4-amino-1-(6-methyl-1,2,3,4-tetrahydroquinolin-3-yl)-1H-pyrazolo[3,4-d]pyrimidin-3-yl)-5-fluorophenol

To a solution of 3-iodo-1-(6-methyl-1, 2, 3, 4-tetrahydroquinolin-3-yl)-1H-pyrazolo[3,4-d]pyrimidin-4-amine (250 mg, 0.496 mmol) in DMF (5 mL) was added 3-fluoro-5-hydroxyphenylboronic acid (155 mg, 0.994 mmol) at RT. Then, Na₂CO₃ (158 mg, 1.490 mmol) dissolved in water (5 mL) was added to the reaction mixture followed by addition of Pd(PPh₃)₄ (57 mg, 0.049 mmol) at RT and the resultant reaction mixture was heated at 90° C. for 16 h. The reaction was monitored by TLC and through LCMS. After 16 h, the reaction mixture was diluted with water (20 mL) and extracted with EtOAc (3×25 mL). The combined organic layers were washed with water (2×50 mL), brine (30 mL), dried over sodium sulfate and concentrated. The crude compound was purified by flash chromatography using 4% MeOH/DCM as eluent to afford 140 mg of 3-(4-amino-1-(6-methyl-1, 2, 3, 4-tetrahydroquinolin-3-yl)-1H-pyrazolo[3,4-d]pyrimidin-3-yl)-5-fluorophenol as an off-white solid.

### Step-6: Synthesis of 1-(3-(4-amino-3-(3-fluoro-5-hydroxyphenyl)-1H-pyrazolo[3,4-d]pyrimidin-1-yl)-6-methyl-3,4-dihydroquinolin-1(2H)-yl)prop-2-en-1-one

To a stirred solution of 3-(4-amino-1-(6-methyl-1, 2, 3, 4-tetrahydroquinolin-3-yl)-1H-pyrazolo[3,4-d]pyrimidin-3-yl)-5-fluorophenol (120 mg, 0.307 mmol) in DCM (4 mL) was added triethylamine (37.3 mg, 0.368 mmol) dissolved in DCM (0.5 mL) slowly at 0° C. Then, acryloyl chloride (27.8 mg, 0.307 mol) dissolved in DCM (0.5 mL) was added dropwise to the reaction mixture at 0° C. and the mixture was stirred for 5 min. The reaction was monitored by TLC. After 5 min, the reaction mixture was diluted with water (40 mL) and extracted with DCM (3×50 mL). The combined organic layers were washed with brine (25 mL), dried over sodium sulfate and concentrated. The crude compound was purified by preparative HPLC affording 3.5 mg of 1-(3-(4-amino-3-(3-fluoro-5-hydroxyphenyl)-1H-pyrazolo[3,4-d]pyrimidin-1-yl)-6-methyl-3,4-dihydroquinolin-1(2H)-yl)prop-2-en-1-one as an off-white solid. ¹H NMR (400 MHz, CDCl₃) δ (ppm): 8.33 (s, 1H), 7.22 (s, 1H), 7.03 (d, J=7.9 Hz, 3H), 6.92-6.82 (m, 2H), 6.63 (d, J=10.1 Hz, 1H), 6.53 (dd, J=17.1, 9.9 Hz, 1H), 6.34 (d, J=16.3 Hz, 1H), 5.64 (d, J=12.1 Hz, 2H), 5.45 (p, J=6.7 Hz, 1H), 4.52 (dd, J=12.9, 6.6 Hz, 1H), 4.05 (ddd, J=16.3, 11.5, 5.7 Hz, 1H), 3.50 (dd, J=16.2, 7.5 Hz, 1H), 3.31 (dd, J=16.3, 6.9 Hz, 1H), 2.34 (s, 3H). Separation by chiral HPLC provides Compound Nos. 31a and 31b.

### Example 32: Preparation of Compound Nos. 32, 32a and 32b

### Step-1: Synthesis of 3-iodo-1-(6-methyl-1,2,3,4-tetrahydroquinolin-3-yl)pyrazolo[3,4-d]pyrimidin-4-amine

See Example No. 31.

### Step-2: Synthesis of 3-(5-fluoro-6-methoxypyridin-3-yl)-1-(6-methyl-1,2,3,4-tetrahydroquinolin-3-yl)-1H-pyrazolo[3,4-d]pyrimidin-4-amine

Na₂CO₃ (95 mg, 0.896 mmol) dissolve in water (3 mL) was added to a solution of 3-iodo-1-(6-methyl-1,2,3,4-tetrahydroquinolin-3-yl)pyrazolo[3,4-d]pyrimidin-4-amine (150 mg, 0.298 mmol) and 3-fluoro-2-methoxy-5-(4,4,5,5-tetramethyl-1,3,2-dioxaborolan-2-yl)pyridine (151 mg, 0.596 mmol) in DMF (3 mL), followed by addition of tetrakis(triphenylphosphine)palladium(0) (34 mg, 0.029 mmol). The reaction was heated at 100° C. overnight, the reaction monitored by LCMS. The mixture was diluted with water (10 mL) and extracted with EtOAc (3×15 mL). The combined organic layers were washed with water (2×40 mL), dried over anhydrous sodium sulfate and concentrated under reduced pressure to afford a crude product, which was purified by flash chromatography using 6% MeOH in DCM as eluent obtain 50 mg of 3-(5-fluoro-6-methoxy-3-pyridyl)-1-(6-methyl-1,2,3,4-tetrahydroquinolin-3-yl)pyrazolo[3,4-d]pyrimidin-4-amine as a white solid.

### Step-3: Synthesis of 1-(3-(4-amino-3-(5-fluoro-6-methoxypyridin-3-yl)-1H-pyrazolo[3,4-d]pyrimidin-1-yl)-6-methyl-3,4-dihydroquinolin-1 (2H)-yl)prop-2-en-1-one

Triethylamine (0.026 mL, 0.186 mmol) was added to a solution of 3-(5-fluoro-6-methoxy-3-pyridyl)-1-(6-methyl-1,2,3,4-tetrahydroquinolin-3-yl)pyrazolo[3,4-d]pyrimidin-4-amine (50 mg, 0.123 mmol) in DCM (4 mL). Followed by dropwise addition of prop-2-enoyl chloride (12 mg, 0.132 mmol) in DCM (1 mL) at 0° C. The reaction was allowed to stir at 0° C. for 30 min, the reaction monitored by TLC. The reaction was quenched with aq. sodium bicarbonate solution (5 mL) and extracted with DCM (2×10 mL). The combined organic layer was dried over anhydrous sodium sulfate and concentrated under reduced pressure to afford a crude product, which was purified by reverse phase preparative HPLC to afford 8 mg of 1-[3-[4-amino-3-(5-fluoro-6-methoxy-3-pyridyl)pyrazolo[3,4-d]pyrimidin-1-yl]-6-methyl-3,4-dihydro-2H-quinolin-1-yl]prop-2-en-1-one as a white solid. ¹HNMR (400 MHz, DMSO-d₆) δ (ppm): 8.27 (s, 1H), 8.05 (s, 1H), 7.69 (d, J=10.9 Hz, 1H), 7.23-6.92 (m, 3H), 6.48 (dd, J=16.8, 10.3 Hz, 1H), 6.07 (d, J=16.6 Hz, 1H), 5.61 (d, J=10.3 Hz, 1H), 5.40 (p, J=5.7 Hz, 1H), 4.35 (dd, J=13.1, 5.8 Hz, 1H), 3.99 (s, 3H), 3.91 (d, J=13.8 Hz, 1H), 3.22-3.10 (m, 2H), 2.29 (s, 3H). Separation by chiral HPLC provides Compound Nos. 32a and 32b.

### Example 33: Preparation of Compound Nos. 33, 33a and 33b

### Synthesis of 3-(3,4-dimethoxyphenyl)-1-(1,2,3,4-tetrahydronaphthalen-1-yl)-1H-pyrazolo[3,4-d]pyrimidin-4-amine

Steps 1-3 are same as in Example 12

### Step-4: Synthesis of 3-(3,4-dimethoxyphenyl)-1-(1,2,3,4-tetrahydronaphthalen-1-yl)-1H-pyrazolo[3,4-d]pyrimidin-4-amine

To a suspension of 3-iodo-1-tetralin-1-yl-pyrazolo[3,4-d]pyrimidin-4-amine (100 mg, 0.255 mmol) and (3,4-dimethoxyphenyl)boronic acid (70 mg, 0.382 mmol) in DMF (2 mL) was added a solution of sodium carbonate (54 mg, 0.510 mmol) in water (2 mL) followed by the addition of tetrakis(triphenylphosphine)palladium(0) (29 mg, 0.0255 mmol). The reaction mixture was heated in a reagent bottle at 100° C. overnight. The progress of reaction was monitored by TLC. After completion of reaction, water (20 mL) was added to the reaction mixture and the product was extracted with EtOAc (2×25 mL). The combined organic layers were washed with water (2×15 mL) and then with brine solution (15 mL). The organic layer was separated, dried over anhydrous sodium sulfate and concentrated under reduced pressure to afford a crude product which was purified by reverse phase preparative HPLC to afford 3-(3,4-dimethoxyphenyl)-1-tetralin-1-yl-pyrazolo[3,4-d]pyrimidin-4-amine (36 mg) as an off-white solid. ¹HNMR (400 MHz, CDCl₃) δ (ppm): 8.41 (s, 1H), 7.20-7.10 (m, 4H), 7.04-6.92 (m, 2H), 6.66 (d, J=7.8 Hz, 1H), 6.23 (dd, J=9.7, 5.7 Hz, 1H), 5.58 (bs, 2H), 3.92 (s, 6H), 3.05 (m, 1H), 2.88 (m, 1H), 2.64-2.43 (m, 1H), 2.28 (m, 2H), 1.99 (m, 1H). LCMS: 402.5 (M+1). Separation by chiral HPLC affords Compound Nos. 33a and 33b.

### Example 34: Preparation of Compound Nos. 34, 34a and 34b

### Synthesis of 1-(2,3-dihydro-1H-inden-1-yl)-3-(3,4-dimethoxyphenyl)-1H-pyrazolo[3,4-d]pyrimidin-4-amine

Steps 1-2 are same as in Example 10

### Step-3: Synthesis of 1-(2,3-dihydro-1H-inden-1-yl)-3-(3,4-dimethoxyphenyl)-1H-pyrazolo[3,4-d]pyrimidin-4-amine

To a solution of 1-indan-1-yl-3-iodo-pyrazolo[3,4-d]pyrimidin-4-amine (100 mg, 0.265 mmol) and (3,4-dimethoxyphenyl)boronic acid (72 mg, 0.397 mmol) in DMF (2 mL) was added a solution of sodium carbonate (56 mg, 0.530 mmol) in water (2 mL) followed by the addition of tetrakis(triphenylphosphine)palladium(0) (31 mg, 0.0265 mmol). The reaction mixture was heated in a reagent bottle at 100° C. overnight. The progress of reaction was monitored by TLC. After completion of reaction, water (20 mL) was added to the reaction mixture and the product was extracted with EtOAc (2×25 mL). The combined organic layer was again washed with water (2×15 mL) and then with brine solution (15 mL). The organic layer was separated, dried over anhydrous sodium sulfate and concentrated under reduced pressure to afford a crude product which was purified by reverse phase preparative HPLC to afford 3-(3,4-dimethoxyphenyl)-1-indan-1-yl-pyrazolo[3,4-d]pyrimidin-4-amine (25 mg) as an off-white solid. ¹HNMR (400 MHz, CDCl₃) δ (ppm): 8.42 (s, 1H), 7.31 (d, J=7.5 Hz, 1H), 7.23 (d, J=7.4 Hz, 1H), 7.21-7.07 (m, 3H), 7.04 (d, J=7.5 Hz, 1H), 6.97 (d, J=8.2 Hz, 1H), 6.53 (t, J=7.7 Hz, 1H), 5.50 (bs, 2H), 3.92 (s), 3.35 (m, 1H), 3.06 (m, 1H), 2.86-2.64 (m, 2H). LCMS: 388.5 (M+1). Separation by chiral HPLC affords Compound Nos. 34a and 34b.

### Example 35: Preparation of Compound No. 35

### Synthesis of 1-(2,3-dihydro-1H-inden-2-yl)-3-(3,4-dimethoxyphenyl)-1H-pyrazolo[3,4-d]pyrimidin-4-amine

Steps 1-5 are same as in Example 3

### Step-6: Synthesis of 1-(2, 3-dihydro-1H-inden-2-yl)-3-(3,4-dimethoxyphenyl)-1H-pyrazolo[3,4-d]pyrimidin-4-amine

To a solution of 1-indan-2-yl-3-iodo-pyrazolo[3,4-d]pyrimidin-4-amine (100 mg, 0.265 mmol) and (3,4-dimethoxyphenyl)boronic acid (72 mg, 0.397 mmol) in DMF (2 mL) was added a solution of sodium carbonate (56 mg, 0.530 mmol) in water (2 mL) followed by the addition of tetrakis(triphenylphosphine)palladium(0) (31 mg, 0.0265 mmol). The reaction mixture was heated in a reagent bottle at 100° C. overnight. The progress of reaction was monitored by TLC. After completion of reaction, water (20 mL) was added to the reaction mixture and the product was extracted with EtOAc (2×20 mL). The combined organic layer was again washed with water (2×15 mL) and then with brine solution (15 mL). The organic layer was separated, dried over anhydrous sodium sulfate and concentrated under reduced pressure to afford a crude product (140 mg) which was purified by HPLC to obtain 1-(2,3-dihydro-1H-inden-2-yl)-3-(3,4-dimethoxyphenyl)-1H-pyrazolo[3,4-d]pyrimidin-4-amine (10 mg) as an off-white solid. ¹HNMR (400 MHz, CDCl₃) δ (ppm): 8.4 (s, 1H), 7.25-7.19 (m, 6H), 7.02 (d, J=7.5 Hz, 1H), 5.83 (m, 1H), 5.65 (bs, 1H), 3.92 (s), 3.7 (m, 2H), 3.45 (m, 2H). LCMS: 388.1 (M+1).

### Example 36: Preparation of Compound Nos. 36, 36a and 36b

### Synthesis of 5-[4-amino-1-(2,3-dihydro-1H-inden-1-yl)-1H-pyrazolo[3,4-d]pyrimidin-3-yl]-2-bromophenol

### Step-1: Synthesis of 4-bromo-3-methoxyphenylboronic acid

To a stirred solution of 1-bromo-4-iodo-2-methoxybenzene (2.4 g, 7.66 mmol) in diethyl ether (25 mL) was added n-BuLi (2M in hexane; 4.40 mL, 8.81 mmol) at −78° C. dropwise. The reaction mixture was stirred at −78° C. for 30 min, and then triisopropyl borate (2.03 mL, 8.81 mmol) was slowly added to the reaction mixture at −78° C. The temperature of the reaction mixture was gradually raised to RT over a period of 1 h, and then the reaction mixture was stirred at RT for 30 min. After 30 min, 3 N HCl (25 mL) was slowly added to the reaction mixture at 0° C. and stirred for 1 h. The progress of reaction was monitored by TLC. After completion of reaction, the reaction mixture was diluted with water (150 mL) and the product was extracted with EtOAc (2×200 mL). The combined organic layers were washed with brine (100 mL), dried over sodium sulfate and concentrated. The residue obtained was triturated with hexane (2×25 mL) to afford 4-bromo-3-methoxyphenylboronic acid as an off-white solid (1.45 g).

### Step-2: Synthesis of 4-bromo-3-hydroxyphenylboronic acid

To a stirred suspension of 4-bromo-3-methoxyphenylboronic acid (1.3 g, 5.63 mmol) in DCM (20 mL) was added BBr₃ (1 M in DCM; 28.15 mL, 28.15 mmol) at 0° C. dropwise. The resultant reaction mixture was stirred at RT for 6 h. After completion of reaction, the reaction was quenched with ice-cold water and then acidified to pH 1 using 6 N—HCl. The aq. layer was then extracted with EtOAc (2×200 mL). The combined organic layers were washed with water (100 mL) then with brine (80 mL), dried over sodium sulfate and concentrated. The residue obtained was triturated with pentane (25 mL) to afford 4-bromo-3-hydroxyphenylboronic acid (1.1 g) as an off-white solid.

### Step-3: Synthesis of 5-(4-amino-1-(2,3-dihydro-1H-inden-1-yl)-1H-pyrazolo[3,4-d]pyrimidin-3-yl)-2-bromophenol

To a solution of 1-(2, 3-dihydro-1H-inden-1-yl)-3-iodo-1H-pyrazolo[3,4-d]pyrimidin-4-amine (200 mg, 0.530 mmol) in DMF (4 mL) was added 4-bromo-3-hydroxyphenylboronic acid (172.46 mg, 0.795 mmol) at RT. Then, Na₂CO₃ (168.5 mg, 1.59 mmol) dissolved in water (4 mL) was added to the reaction mixture followed by addition of Pd(PPh₃)₄ (30.63 mg, 0.26 mmol) at RT and the resultant reaction mixture was heated at 70° C. for 6 h. The progress of reaction was monitored by TLC and LCMS. After completion of reaction, the reaction mixture was cooled to RT and diluted with water (15 mL). The aq. layer was then extracted with EtOAc (2×50 mL). The combined organic layers were washed with water (20 mL) then brine (20 mL), dried over sodium sulfate and concentrated. The crude compound was purified by preparative HPLC to afford 5-(4-amino-1-(2,3-dihydro-1H-inden-1-yl)-1H-pyrazolo[3,4-d]pyrimidin-3-yl)-2-bromophenol (70 mg) as a white solid. ¹HNMR (400 MHz, DMSO-d6) δ (ppm): 8.30 (s, 1H), 7.58 (d, J=8.2 Hz, 1H), 7.35 (d, J=7.5 Hz, 1H), 7.25 (t, J=7.4 Hz, 1H), 7.18 (d, J=2.0 Hz, 1H), 7.12 (t, J=7.5 Hz, 1H), 7.01-6.81 (m, 2H), 6.44 (t, J=7.5 Hz, 1H), 3.23 (ddd, J=16.0, 8.8, 4.5 Hz, 1H), 3.02 (dt, J=15.9, 7.9 Hz, 1H), 2.63 (dtd, J=20.2, 12.9, 4.7 Hz, 2H). Separation by chiral HPLC affords Compound Nos. 36a and 36b.

### Example 37: Preparation of Compound Nos. 37, 37a and 37b

### Synthesis of 1-(2,3-dihydro-1H-inden-1-yl)-3-(1H-indol-6-yl)-1H-pyrazolo[3,4-d]pyrimidin-4-amine

Steps 1-5 are same as in Example 10.

### Step-6: Synthesis of 1-(2,3-dihydro-1H-inden-1-yl)-3-(1H-indol-6-yl)-1H-pyrazolo[3,4-d]pyrimidin-4-amine

To a solution of 1-(2,3-dihydro-1H-inden-1-yl)-3-iodo-1H-pyrazolo[3,4-d]pyrimidin-4-amine (200 mg, 0.530 mmol) in DMF (4 mL) was added 1H-indol-6-ylboronic acid (128.03 mg, 0.795 mmol) at RT. Then, Na₂CO₃ (168.58 mg, 1.59 mmol) dissolved in water (4 mL) was added to the reaction mixture followed by addition of Pd(PPh₃)₄ (61.2 mg, 0.053 mmol) at RT and the resultant reaction mixture was stirred at 100° C. for 16 h. The progress of reaction was monitored by TLC. After completion the reaction, the reaction mixture was cooled to RT and diluted with water (100 mL). The aq. layer was then extracted with EtOAc (2×100 mL). The combined organic layers were washed with brine (30 mL), dried over sodium sulfate and concentrated. The crude compound was purified by preparative HPLC affording 1-(2,3-dihydro-1H-inden-1-yl)-3-(1H-indol-6-yl)-1H-pyrazolo[3,4-d]pyrimidin-4-amine (56 mg) as an off-white solid. ¹HNMR (400 MHz, DMSO-d6) δ (ppm): 11.25 (s, 1H), 8.30 (s, 1H), 7.70-7.55 (m, 2H), 7.43 (s, 1H), 7.34 (d, J=7.6 Hz, 1H), 7.25 (dd, J=8.1, 4.4 Hz, 2H), 7.12 (t, J=7.4 Hz, 1H), 6.95 (d, J=7.5 Hz, 1H), 6.46 (dd, J=15.5, 8.0 Hz, 3H), 3.31-3.02 (m, 2H), 2.64 (hept, J=6.8, 5.8 Hz, 2H). Separation by chiral HPLC affords Compound Nos. 37a and 37b.

### Example 38: Preparation of Compound Nos. 38, 38a and 38b

### Synthesis of 3-(1H-indol-6-yl)-1-(1,2,3,4-tetrahydronaphthalen-1-yl)-1H-pyrazolo[3,4-d]pyrimidin-4-amine

Steps 1-5 are same as Example 12.

### Step-7: Synthesis of 3-(1H-indol-6-yl)-1-(1,2,3,4-tetrahydronaphthalen-1-yl)-1H-pyrazolo[3,4-d]pyrimidin-4-amine

To a solution of 3-iodo-1-(1,2,3,4-tetrahydronaphthalen-1-yl)-1H-pyrazolo[3,4-d]pyrimidin-4-amine (200 mg, 0.511 mmol) in DMF (4 mL) was added 1H-indol-6-ylboronic acid (123.4 mg, 0.766 mmol) at RT. Then, Na₂CO₃ (162.5 mg, 1.53 mmol) dissolved in water (4 mL) was added to the reaction mixture followed by addition of Pd(PPh₃)₄ (59.07 mg, 0.05 mmol) at RT and the resultant reaction mixture was stirred at 100° C. for 16 h. The progress of reaction was monitored by TLC. After completion of reaction, the reaction mixture was cooled to RT and diluted with water (100 mL). The aq. layer was then extracted with EtOAc (2×100 mL). The combined organic layers were washed with brine (30 mL), dried over sodium sulfate and concentrated to obtain a crude product which was purified by preparative HPLC to afford 3-(1H-indol-6-yl)-1-(1,2,3,4-tetrahydronaphthalen-1-yl)-1H-pyrazolo[3,4-d]pyrimidin-4-amine (20 mg) as an off-white solid. ¹HNMR (400 MHz, DMSO-d6) δ (ppm): 11.25 (s, 1H), 8.29 (s, 1H), 7.77-7.48 (m, 2H), 7.21 (m, 2H), 7.15 (q, J=7.9 Hz, 2H), 7.01 (t, J=7.5 Hz, 1H), 6.57 (d, J=7.8 Hz, 1H), 6.49 (s, 1H), 6.19 (m, 1H), 3.05-2.82 (m, 2H), 2.40 (q, J=9.7 Hz, 1H), 2.30-2.03 (m, 2H), 1.92 (d, J=8.3 Hz, 1H). Separation by chiral HPLC affords Compound Nos. 38a and 38b.

### Example 39: Preparation of Compound No. 39

### Synthesis of 1-(2,3-dihydro-1H-inden-2-yl)-3-(1H-indol-6-yl)-1H-pyrazolo[3,4-d]pyrimidin-4-amine

Steps 1-5 are same as in Example 3.

### Step-6: Synthesis of 1-(2, 3-dihydro-1H-inden-2-yl)-3-(1H-indol-6-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (200 mg, 0.530 mmol) in DMF (4 mL) was added 1H-indol-6-ylboronic acid (128.03 mg, 0.795 mmol) at RT. Then, Na₂CO₃ (168.58 mg, 1.59 mmol) dissolved in water (4 mL) was added to the reaction mixture followed by addition of Pd(PPh₃)₄ (61.2 mg, 0.053 mmol) at RT and the resultant reaction mixture was stirred at 100° C. for 16 h. The progress of reaction was monitored by TLC. After completion of reaction, the reaction mixture was cooled to RT and diluted with water (100 mL). The aq. layer was then extracted with EtOAc (2×100 mL). The combined organic layers were washed with brine (30 mL), dried over sodium sulfate and concentrated to obtain a crude product which was purified by preparative HPLC to afford 1-(2,3-dihydro-1H-inden-2-yl)-3-(1H-indol-6-yl)-1H-pyrazolo[3,4-d]pyrimidin-4-amine (92 mg) as a white solid. ¹HNMR (400 MHz, DMSO-d6) δ (ppm): 11.29 (s, 1H), 8.27 (s, 1H), 7.68 (d, J=9.5 Hz, 2H), 7.44 (m, 2H), 7.28 (t, J=7.6 Hz, 3H), 7.23-7.04 (m, 2H), 6.50 (s, 2H), 5.74 (p, J=8.0 Hz, 1H), 3.55-3.25 (m, 4H).

### Example 40: Preparation of Compound No. 40

### Synthesis of 3-[4-amino-1-(2,3-dihydro-1H-inden-2-yl)-1H-pyrazolo[3,4-d]pyrimidin-3-yl]phenol

Steps 1-5 are same as in Example 3.

### Step 6: Synthesis of 2-(3-methoxyphenyl)-4,4,5,5-tetramethyl-1,3,2-dioxaborolane

To a stirred solution of 1-bromo-3-methoxy-benzene (500 mg, 2.67 mmol) and bis(pinacolato)diboron (814 mg, 3.20 mmol) in dioxane (10 mL) was added potassium acetate (787 mg, 8.01 mmol) and reaction was purged with nitrogen for 30 min. After 30 min Pd(dppf)Cl₂.DCM (218 mg, 0.26 mmol) was added to the reaction mixture and reaction was heated at 100° C. overnight. The progress of the reaction was monitored by TLC. After completion of reaction, the reaction mixture was cooled to RT, then filtered through a celite bed and the celite bed was washed with EtOAc (200 mL). The filtrate was concentrated under reduced pressure to obtain 2-(3-methoxyphenyl)-4,4,5,5-tetramethyl-1,3,2-dioxaborolane (550 mg) as a crude compound which was used in the next step without further purification.

### Step 7: Synthesis of 1-indan-2-yl-3-(3-methoxyphenyl)pyrazolo[3,4-d]pyrimidin-4-amine

To a solution of 1-indan-2-yl-3-iodo-pyrazolo[3,4-d]pyrimidin-4-amine (500 mg, 1.32 mmol) and 2-(3-methoxyphenyl)-4,4,5,5-tetramethyl-1,3,2-dioxaborolane (465 mg, 1.98 mmol) in N,N-dimethylformamide (6 mL) was added a solution of sodium carbonate (280 mg, 2.65 mmol) in water (6 mL) followed by the addition of tetrakis(triphenylphosphine) palladium(0) (153 mg, 0.132 mmol). The reaction mixture was heated in a reagent bottle at 100° C. overnight. The reaction was monitored by TLC. After completion of reaction, water (30 mL) was added to the reaction mixture and the product was extracted using EtOAc (2×150 mL). The combined organic layer was again washed with water (2×50 mL) and brine (50 mL). The organic layer was separated, dried over anhydrous sodium sulfate and concentrated to obtain a crude product which was purified by triturating with pentane and diethyl ether to obtain 1-indan-2-yl-3-(3-methoxyphenyl)pyrazolo[3,4-d]pyrimidin-4-amine (390 mg) as a brown solid.

### Step 8: Synthesis of 3-(4-amino-1-indan-2-yl-pyrazolo[3,4-d]pyrimidin-3-yl)phenol

To a stirred solution of 1-indan-2-yl-3-(3-methoxyphenyl)pyrazolo[3,4-d]pyrimidin-4-amine (100 mg, 0.279 mmol) in DCM (4 mL) was added dropwise BBr₃ (1.39 mL, 1.39 mmol, 1 M solution in DCM) at RT under nitrogen atmosphere. The reaction mixture was stirred at RT for 1 hr. The reaction was monitored by TLC. After completion of reaction, the reaction mixture was concentrated under reduced pressure. To the residue was added sat. aq. NaHCO₃ solution (5 mL) and the product was extracted using EtOAc (30 mL). The organic layer was again washed with water (10 mL) and finally with brine (10 mL). The organic layer was separated, dried over anhydrous sodium sulfate and concentrated under reduced pressure to afford the crude product which was purified using reverse phase preparative HPLC to get 3-(4-amino-1-indan-2-yl-pyrazolo[3,4-d]pyrimidin-3-yl)phenol (34.64 mg) as a light brown solid. ¹HNMR (400 MHz, Methanol-d4) δ (ppm): 8.26 (s, 1H), 7.36 (t, J=7.9 Hz, 1H), 7.25 (m, 2H), 7.23-7.16 (m, 2H), 7.14-7.02 (m, 2H), 6.96-6.86 (m, 1H), 5.77 (m, 1H), 3.62 (dd, J=15.8, 8.2 Hz, 2H), 3.45 (dd, J=15.7, 8.3 Hz, 2H). LCMS 344.1 (m+1).

### Example 41: Preparation of Compound Nos. 41, 41a and 41b

### Synthesis of 3-[4-amino-1-(7-fluoro-1,2,3,4-tetrahydronaphthalen-1-yl)-1H-pyrazolo[3,4-d]pyrimidin-3-yl]-5-fluorophenol

To a solution of 1-(7-fluorotetralin-1-yl)-3-iodo-pyrazolo[3,4-d]pyrimidin-4-amine (270 mg, 0.65 mmol) and (3-fluoro-5-hydroxy-phenyl)boronic acid (153 mg, 0.98 mmol) in N,N-dimethylformamide (3 mL) was added a solution of sodium carbonate (139 mg, 1.31 mmol) in water (3 mL) followed by the addition of tetrakis(triphenylphosphine)palladium(0) (76 mg, 0.06 mmol). The reaction mixture was heated in a reagent bottle at 100° C. overnight. The progress of reaction was monitored by TLC. After completion of reaction, water (40 mL) was added to the reaction mixture and the product was extracted using EtOAc (2×75 mL). The combined organic layer was again washed with water (2×50 mL) and brine (50 mL). The organic layer was separated, dried over anhydrous sodium sulfate and concentrated to obtain a crude product which was purified by reverse phase preparative HPLC to obtain 3-[4-amino-1-(7-fluorotetralin-1-yl)pyrazolo[3,4-d]pyrimidin-3-yl]-5-fluoro-phenol (39.35 mg) as off white solid. ¹HNMR (400 MHz, Methanol-d4) δ (ppm): 8.31 (s, 1H), 7.20 (dd, J=8.6, 5.8 Hz, 1H), 6.95-6.80 (m, 2H), 6.63 (dt, J=10.7, 2.3 Hz, 1H), 6.28 (dd, J=9.8, 2.7 Hz, 1H), 6.14 (m, 1H), 3.06-2.94 (m, 1H), 2.87 (m, 1H), 2.52-2.35 (m, 1H), 2.26 (m, 2H), 2.06-1.89 (m, 1H), 1.29 (s, 1H). LCMS 394.1 (M+1). Separation by chiral HPLC affords Compound Nos. 41a and 41b.

### Example 42: Preparation of Compound Nos. 42, 42a and 42b

### Synthesis of 3-[4-amino-1-(5-fluoro-2,3-dihydro-1H-inden-1-yl)-1H-pyrazolo[3,4-d]pyrimidin-3-yl]-5-fluorophenol

### Step-1: Synthesis of 1-(5-fluoroindan-1-yl)-3-iodo-pyrazolo[3,4-d]pyrimidin-4-amine

To a stirred solution of 3-iodo-1H-pyrazolo[3,4-d]pyrimidin-4-amine (350 mg, 1.34 mmol) in DMF (8 mL) was added Cs₂CO₃ (873.7 mg, 2.68 mmol) at RT and the reaction mixture was allowed to stir at RT for 30 min. Then, TBAI (99.05 mg, 0.268 mmol) and 5-fluoro-2,3-dihydro-1H-inden-1-yl methanesulfonate (617.5 mg, 2.68 mmol) dissolved in DMF (4 mL) was added to the reaction mixture and the resultant reaction mixture was heated at 75° C. for 1.5 h. The progress of reaction was monitored by TLC. After completion of reaction, the reaction mixture was cooled to RT and diluted with water (100 mL). The aqueous layer was extracted with EtOAc (2×200 mL). The combined organic layers were washed with water (100 mL), brine (100 mL), dried over Na₂SO₄ and concentrated to obtain a crude product which was purified by column chromatography on silica-gel using 10% acetone-hexane system as eluent to afford 1-(5-fluoroindan-1-yl)-3-iodo-pyrazolo[3,4-d]pyrimidin-4-amine (210 mg) as a light brown solid compound.

### Step-2: Synthesis of 3-(4-amino-1-(5-fluoro-2,3-dihydro-1H-inden-1-yl)-1H-pyrazolo[3,4-d]pyrimidin-3-yl)-5-fluorophenol

To a solution of 1-(5-fluoro-2,3-dihydro-1H-inden-1-yl)-3-iodo-1H-pyrazolo[3,4-d]pyrimidin-4-amine (200 mg, 0.506 mmol) in DMF (4 mL) was added 3-fluoro-5-hydroxyphenylboronic acid (118.36 mg, 0.759 mmol) at RT. Then, Na₂CO₃ (160.9 mg, 1.51 mmol) dissolved in water (4 mL) was added to the reaction mixture followed by addition of Pd(PPh₃)₄ (58.4 mg, 0.050 mmol) at RT and the resultant reaction mixture was heated at 100° C. for 16 h. The progress of reaction was monitored by TLC. After completion of reaction, the reaction mixture was cooled to RT and diluted with water (20 mL). The aq. layer was then extracted with EtOAc (2×50 mL). The combined organic layers were washed with water (25 mL), brine (25 mL), dried over sodium sulfate and concentrated to obtain a crude product which was purified by preparative HPLC to afford 3-(4-amino-1-(5-fluoro-2,3-dihydro-1H-inden-1-yl)-1H-pyrazolo[3,4-d]pyrimidin-3-yl)-5-fluorophenol (51 mg) as an off-white solid. ¹HNMR (400 MHz, DMSO-d6) δ (ppm): 8.30 (s, 1H), 7.21-7.13 (m, 1H), 7.01-6.88 (m, 2H), 6.87-6.82 (m, 1H), 6.79 (dt, J=9.3, 1.9 Hz, 1H), 6.63 (dt, J=11.0, 2.3 Hz, 1H), 6.40 (t, J=7.3 Hz, 1H), 3.23 (dt, J=8.8, 3.7 Hz, 1H), 3.03 (dt, J=16.0, 7.7 Hz, 1H), 2.75-2.57 (m, 2H). Separation by chiral HPLC affords Compound Nos. 42a and 42b.

### Example 43: Preparation of Compound No. 43

### Synthesis of 3-[3-(benzyloxy)-4-(trifluoromethyl)phenyl]-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (150 mg, 0.39 mmol) and [3-benzyloxy-4-(trifluoromethyl)phenyl]boronic acid (178 mg, 0.59 mmol) in DMF (5 mL) was added a solution of sodium carbonate (127 mg, 1.19 mmol) in water (5 mL) followed by the addition of tetrakis(triphenylphosphine)palladium(0) (46 mg, 0.039 mmol). The reaction mixture was heated in a reagent bottle at 100° C. overnight. The progress of reaction was monitored by TLC. After completion of reaction, water (20 mL) was added to the reaction mixture and the product was extracted with (2×20 mL). The combined organic layer was again washed with water (2×15 mL) and finally with brine solution (15 mL). The organic layer was separated, dried over anhydrous sodium sulfate and concentrated under reduced pressure to afford a crude product which was purified by reverse phase HPLC to give 3-[3-benzyloxy-4-(trifluoromethyl)phenyl]-1-indan-2-yl-pyrazolo[3,4-d]pyrimidin-4-amine (18 mg) as an off-white solid, which was dissolved in 5 mL ethanolic HCl and concentrated under reduced pressure to obtain 3-[3-benzyloxy-4-(trifluoromethyl)phenyl]-1-indan-2-yl-pyrazolo[3,4-d]pyrimidin-4-amine hydrochloride salt (19.5 mg) as an off-white solid. ¹HNMR (400 MHz, Methanol-d4) δ (ppm): 8.34 (s, 1H), 7.77 (d, J=8.0 Hz, 1H), 7.52 (s, 1H), 7.46 (m, 2H), 7.42-7.30 (m, 4H), 7.29-7.15 (m, 4H), 5.82 (m, 1H), 5.29 (s, 2H), 3.62 (dd, J=15.9, 7.8 Hz, 2H), 3.49 (dd, J=15.9, 8.4 Hz, 2H).

### Example 44: Preparation of Compound No. 44

### Synthesis of 5-[4-amino-1-(2,3-dihydro-1H-inden-2-yl)-1H-pyrazolo[3,4-d]pyrimidin-3-yl]-2-methylphenol

To a solution of 1-indan-2-yl-3-iodo-pyrazolo[3,4-d]pyrimidin-4-amine (150 mg, 0.39 mmol) and (3-hydroxy-4-methyl-phenyl)boronic acid (91 mg, 0.59 mmol) in DMF (5 mL) was added a solution of sodium carbonate (127 mg, 1.19 mmol) in water (5 mL) followed by the addition of tetrakis(triphenylphosphine)palladium(0) (46 mg, 0.039 mmol). The reaction mixture was heated in a reagent bottle at 100° C. overnight. The progress of reaction was monitored by TLC. After completion of reaction, water (20 mL) was added to the reaction mixture and the product was extracted with (2×20 mL). The combined organic layer was again washed with water (2×15 mL) then with brine solution (15 mL), dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2-methyl-phenol (40 mg) as an off-white solid. The purified product was dissolved in ethanolic HCl (10 mL) and concentrated under reduced pressure to obtain 5-(4-amino-1-indan-2-yl-pyrazolo[3,4-d]pyrimidin-3-yl)-2-methyl-phenol hydrochloride salt (43 mg) off-white solid. ¹HNMR (400 MHz, Methanol-d4) δ (ppm): 8.40 (s, 1H), 7.27 (m, 3H), 7.21-7.14 (m, 2H), 7.10-6.98 (m, 2H), 5.85 (p, J=8.0 Hz, 1H), 3.62 (dd, J=15.9, 7.7 Hz, 2H), 3.51 (dd, J=15.9, 8.4 Hz, 2H), 2.27 (s, 3H).

### Example 45: Preparation of Compound No. 45

### Synthesis of 3-(3-aminophenyl)-1-(2,3-dihydro-1H-inden-2-yl)-1H-pyrazolo[3,4-d]pyrimidin-4-amine

Steps 1-5 are same as in Example 3

### Step 6: Synthesis of 3-(3-aminophenyl)-1-indan-2-yl-pyrazolo[3,4-d]pyrimidin-4-amine

To a solution of 1-indan-2-yl-3-iodo-pyrazolo[3,4-d]pyrimidin-4-amine (300 mg, 0.79 mmol) and 3-(4,4,5,5-tetramethyl-1,3,2-dioxaborolan-2-yl)aniline (261 mg, 1.19 mmol) in N,N-dimethylformamide (5 mL) was added a solution of sodium carbonate (168 mg, 1.59 mmol) in water (5 mL) followed by the addition of tetrakis(triphenylphosphine)palladium(0) (91 mg, 0.079 mmol). The reaction mixture was heated in a reagent bottle at 100° C. overnight. The progress of reaction was monitored by TLC. After completion of reaction, water (20 mL) was added to the reaction mixture and the product was extracted using EtOAc (2×300 mL). The combined organic layer was again washed with water (4×100 mL) and brine (100 mL), dried over anhydrous sodium sulfate and concentrated to obtain a crude product which was purified by reverse phase preparative HPLC to give 3-(3-aminophenyl)-1-indan-2-yl-pyrazolo[3,4-d]pyrimidin-4-amine (11.1 mg). The purified product was dissolved in ethanolic HCl (2 mL) stirred for 2 min and then ethanolic HCl was evaporated under reduced pressure to give 3-(3-aminophenyl)-1-indan-2-yl-pyrazolo[3,4-d]pyrimidin-4-amine as the HCl salt (12.15 mg). ¹HNMR (400 MHz, Methanol-d4) δ (ppm): 8.46 (s, 1H), 7.73-7.53 (m, 3H), 7.44 (t, J=5.0 Hz, 1H), 7.31-7.12 (m, 4H), 5.90 (m, 1H), 3.62-3.46 (m, 4H) LCMS 343.5 (M+1).

### Example 46: Preparation of Compound No. 46

### Synthesis of 3-(3-amino-4-methylphenyl)-1-(2,3-dihydro-1H-inden-2-yl)-1H-pyrazolo[3,4-d]pyrimidin-4-amine

Steps 1-5 are same as in Example 3

### Step 6: Synthesis of 3-(3-amino-4-methyl-phenyl)-1-indan-2-yl-pyrazolo[3,4-d]pyrimidin-4-amine

To a solution of 1-indan-2-yl-3-iodo-pyrazolo[3,4-d]pyrimidin-4-amine (150 mg, 0.39 mmol) and 2-methyl-5-(4,4,5,5-tetramethyl-1,3,2-dioxaborolan-2-yl)aniline (139 mg, 0.59 mmol) in N,N-dimethylformamide (3 mL) was added a solution of sodium carbonate (84 mg, 0.79 mmol) in water (3 mL) followed by the addition of tetrakis(triphenylphosphine) palladium(0) (45 mg, 0.039 mmol). The reaction mixture was heated in a reagent bottle at 100° C. overnight. The progress of reaction was monitored by TLC. After completion of reaction, water (20 mL) was added to the reaction mixture and the product was extracted using EtOAc (2×100 mL). The combined organic layer was again washed with water (2×50 mL) and brine (50 mL). The organic layer was separated, dried over anhydrous sodium sulfate and concentrated to obtain a crude product which was purified by reverse phase preparative HPLC to give (3-(3-amino-4-methyl-phenyl)-1-indan-2-yl-pyrazolo[3,4-d]pyrimidin-4-amine) (16.5 mg). The purified product was dissolved in ethanolic HCl (2 mL) stirred for 2 min and then ethanolic HCl was evaporated under reduced pressure to give 3-(3-amino-4-methyl-phenyl)-1-indan-2-yl-pyrazolo[3,4-d]pyrimidin-4-amine as the HCl salt (17.9 mg). ¹HNMR (400 MHz, Methanol-d4) δ (ppm): 8.46 (d, J=3.0 Hz, 1H), 7.67-7.45 (m, 3H), 7.34-7.10 (m, 4H), 5.90 (m, 1H), 3.66-3.43 (m, 4H), 2.46 (d, J=2.8 Hz, 3H) LCMS 357.2 (M+1).

### Example 47: Preparation of Compound No. 47

### Synthesis of 3-(1H-1,3-benzodiazol-5-yl)-1-(2,3-dihydro-1H-inden-2-yl)-1H-pyrazolo[3,4-d]pyrimidin-4-amine

Steps 1-5 are same as in Example 3

### Step-6: Synthesis of 3-(1H-benzimidazol-5-yl)-1-indan-2-yl-pyrazolo[3,4-d]pyrimidin-4-amine

To a solution of 1-indan-2-yl-3-iodo-pyrazolo[3,4-d]pyrimidin-4-amine (100 mg, 0.26 mmol) and 5-(4,4,5,5-tetramethyl-1,3,2-dioxaborolan-2-yl)-1H-benzimidazole (97 mg, 0.39 mmol) in N,N-dimethylformamide (5 mL) was added a solution of sodium carbonate (56 mg, 0.53 mmol) in water (5 mL) followed by the addition of tetrakis(triphenylphosphine) palladium(0) (30 mg, 0.026 mmol). The reaction mixture was heated in a reagent bottle at 100° C. overnight. The progress of reaction was monitored by TLC. After completion of reaction, water (20 mL) was added to the reaction mixture and the product was extracted using EtOAc (2×100 mL). The combined organic layer was again washed with water (4×50 mL) and brine (50 mL), dried over anhydrous sodium sulfate and concentrated to obtain a crude product which was purified by reverse phase preparative HPLC to afford 3-(1H-benzimidazol-5-yl)-1-indan-2-yl-pyrazolo[3,4-d]pyrimidin-4-amine (10.32 mg). The purified product was dissolved in ethanolic HCl (2 mL) stirred for 2 min and then ethanolic HCl was evaporated under reduced pressure to give 3-(1H-benzimidazol-5-yl)-1-indan-2-yl-pyrazolo[3,4-d]pyrimidin-4-amine as the HCl (11.51 mg) salt. ¹HNMR (400 MHz, Methanol-d4) δ (ppm): 9.51 (s, 1H), 8.49 (s, 1H), 8.16 (s, 1H), 8.03 (d, J=8.3 Hz, 1H), 7.94 (d, J=8.5 Hz, 1H), 7.27 (dt, J=7.4, 3.6 Hz, 2H), 7.25-7.15 (m, 2H), 5.93 (m, 1H), 3.59 (m, 4H).

### Example 48: Preparation of Compound No. 48

### Synthesis of 1-(2,3-dihydro-1H-inden-2-yl)-3-(morpholin-4-yl)-1H-pyrazolo[3,4-d]pyrimidin-4-amine

To a stirred solution of 1-indan-2-yl-3-iodo-pyrazolo[3,4-d]pyrimidin-4-amine (200 mg, 0.530 mmol) and morpholine (231 mg, 2.65 mmol) in DMF (12 mL) was added copper iodide (40 mg, 0.212 mmol), L-proline (49 mg, 0.424 mmol) and potassium carbonate (144 mg, 1.061 mmol). The reaction mixture was heated at 100° C. overnight. The progress of reaction was monitored by TLC and LCMS. After completion of reaction, the reaction mixture was cooled to RT and diluted with water and the product was extracted with EtOAc (2×50 mL). The combined organic layers were washed with water (2×50 mL), brine solution (50 mL), dried over anhydrous sodium sulfate and concentrated under reduced pressure to obtain a crude product which was purified by reverse phase HPLC to give pure 1-indan-2-yl-3-morpholino-pyrazolo[3,4-d]pyrimidin-4-amine (31 mg). The product was dissolved in ethanolic HCl (5 mL) and concentrated under reduced pressure to afford 1-indan-2-yl-3-morpholino-pyrazolo[3,4-d]pyrimidin-4-amine HCl (32 mg). LCMS: 337.1 (M+1).

### Example 49: Preparation of Compound No. 49

### Synthesis of 1-(2,3-dihydro-1H-inden-2-yl)-3-(3-methoxy-4-methylphenyl)-1H-pyrazolo[3,4-d]pyrimidin-4-amine

To a solution of 1-indan-2-yl-3-iodo-pyrazolo[3,4-d]pyrimidin-4-amine (200 mg, 0.53 mmol) and (3-methoxy-4-methyl-phenyl)boronic acid (132 mg, 0.795 mmol) in DMF (5 mL) was added a solution of sodium carbonate (169 mg, 1.59 mmol) in water (5 mL) followed by the addition of tetrakis(triphenylphosphine) palladium(0) (61 mg, 0.053 mmol). The reaction mixture was heated in a reagent bottle at 100° C. overnight. The progress of reaction was monitored by TLC. After completion of reaction, water (20 mL) was added to the reaction mixture and the product was extracted with (2×50 mL). The combined organic layer was again washed with water (2×20 mL) then, with brine solution (20 mL). The organic layer was separated, dried over anhydrous sodium sulfate and concentrated under reduced pressure to afford a crude product which was purified by reverse phase preparative HPLC to afford 1-indan-2-yl-3-(3-methoxy-4-methyl-phenyl)pyrazolo[3,4-d]pyrimidin-4-amine (12 mg). The product was dissolved in ethanolic HCl (3 mL) and concentrated under reduced pressure to afford 1-indan-2-yl-3-(3-methoxy-4-methyl-phenyl)pyrazolo[3,4-d]pyrimidin-4-amine HCl salt (13.5 mg) as an off-white solid. ¹HNMR (400 MHz, Methanol-d4) δ (ppm): 8.38 (s, 1H), 7.38-7.07 (m, 7H), 5.85 (p, J=8.1 Hz, 1H), 3.88 (s, 3H), 3.63 (dd, J=15.9, 7.8 Hz, 2H), 3.51 (dd, J=15.8, 8.3 Hz, 2H), 2.27 (s, 3H). LCMS: 371 (M+1).

### Example 50: Preparation of Compound No. 50

### Synthesis of 1-(2,3-dihydro-1H-inden-2-yl)-3-(2-methoxypyridin-4-yl)-1H-pyrazolo[3,4-d]pyrimidin-4-amine

Steps 1-5 are same as in Example 3.

### Step-6: Synthesis of 1-(2, 3-dihydro-1H-inden-2-yl)-3-(2-methoxypyridin-4-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (150 mg, 0.397 mmol) in DMF (3 mL) was added 2-methoxypyridin-4-ylboronic acid (91.23 mg, 0.596 mmol) at RT. Then, Na₂CO₃ (126.4 mg, 1.19 mmol) dissolved in water (3 mL) was added to the reaction mixture followed by addition of Pd(PPh₃)₄ (45.95 mg, 0.039 mmol) at RT and the resultant reaction mixture was heated at 90° C. for 16 h. The progress of reaction was monitored by TLC and by LCMS. After completion of reaction, the reaction mixture was diluted with water (20 mL) and the product was extracted with EtOAc (3×25 mL). The combined organic layers were washed with water (2×40 mL), brine (20 mL), dried over sodium sulfate and concentrated to get a crude product which was purified by preparative HPLC affording 1-(2,3-dihydro-1H-inden-2-yl)-3-(2-methoxypyridin-4-yl)-1H-pyrazolo[3,4-d]pyrimidin-4-amine (75 mg) as an off-white solid. The crude product (12 mg) was added to ethanolic HCl (2 mL) and stirred for 30 min at RT. The reaction mixture was then concentrated under reduced pressure and lyophilized to afford 1-(2,3-dihydro-1H-inden-2-yl)-3-(2-methoxypyridin-4-yl)-1H-pyrazolo[3,4-d]pyrimidin-4-amine as the HCl salt (13 mg) off-white solid. ¹HNMR (400 MHz, DMSO-d6) δ (ppm): 8.58 (s, 1H), 8.39 (d, J=2.6 Hz, 1H), 7.91 (dd, J=8.6, 2.6 Hz, 1H), 7.33-7.24 (m, 2H), 7.21 (dt, J=5.7, 3.7 Hz, 2H), 6.98 (d, J=8.6 Hz, 1H), 5.79 (q, J=7.5 Hz, 1H), 3.92 (s, 3H), 3.60-3.37 (m, 4H).

### Example 51: Preparation of Compound Nos. 51, 51a and 51b

### Synthesis of 2-[4-amino-3-(3-fluoro-5-hydroxyphenyl)-1H-pyrazolo[3,4-d]pyrimidin-1-yl]-5-fluoro-2,3-dihydro-1H-inden-1-one

### Step-1: Synthesis of 2-bromo-5-fluoro-2,3-dihydro-1H-inden-1-one

To a stirred suspension of 5-fluoro-2,3-dihydro-1H-inden-1-one (2 g, 13.32 mmol) in HBr in AcOH (12 mL) was added Br2 (0.68 mL, 13.32 mmol) dissolved in AcOH (15 mL) at RT over a period of 20 min. The reaction mixture was then allowed to stir at RT for 20 min. The progress of reaction was monitored by TLC. After completion of reaction, the reaction mixture was basified using saturated NaHCO₃ solution at ice-cold condition and the product was extracted with EtOAc (2×100 mL). The combined organic layers were washed with water (3×75 mL), brine (100 mL), dried over sodium sulfate and concentrated to afford 2-bromo-5-fluoro-2,3-dihydro-1H-inden-1-one (3.1 g) yellow sticky compound which was taken to the next step without further purification.

### Step 2: Synthesis of 2-(4-amino-3-(3-fluoro-5-methoxyphenyl)-1H-pyrazolo[3,4-d]pyrimidin-1-yl)-5-fluoro-2,3-dihydro-1H-inden-1-one

To a stirred suspension of 3-(3-fluoro-5-methoxyphenyl)-1H-pyrazolo[3,4-d]pyrimidin-4-amine (180 mg, 0.694 mmol) in DMF (5 mL) was added Cs₂CO₃ (452.4 mg, 1.38 mmol) at RT and the reaction mixture was allowed to stir for 30 min. Then, TBAI (51.2 g, 0.138 mmol) and 2-bromo-5-fluoro-2,3-dihydro-1H-inden-1-one (349.8 mg, 1.52 mmol) were successively added to the reaction mixture and the resultant reaction mixture was heated at 65° C. for 90 min. The progress of reaction was monitored by TLC. After completion of reaction, the reaction mixture was cooled to RT and diluted with water (50 mL) and extracted with EtOAc (2×100 mL). The combined organic layers were washed with water (2×50 mL), dried over sodium sulfate and concentrated to get a crude product which was triturated with ether-hexane (1:3, 2×5 mL) to afford 2-(4-amino-3-(3-fluoro-5-methoxyphenyl)-1H-pyrazolo[3,4-d]pyrimidin-1-yl)-5-fluoro-2,3-dihydro-1H-inden-1-one (150 mg) as a brown solid, which was taken to the next step without further purification.

### Step 3: Synthesis of 2-(4-amino-3-(3-fluoro-5-hydroxyphenyl)-1H-pyrazolo[3,4-d]pyrimidin-1-yl)-5-fluoro-2,3-dihydro-1H-inden-1-one

To a stirred suspension of 2-(4-amino-3-(3-fluoro-5-methoxyphenyl)-1H-pyrazolo[3,4-d]pyrimidin-1-yl)-5-fluoro-2,3-dihydro-1H-inden-1-one (150 mg, 0.368 mmol) in DCM (4 mL) was added BBr₃ (1 M solution in DCM; 1.47 mL, 1.47 mmol) at 0° C. dropwise and the resultant reaction mixture was stirred at RT for 16 h. The progress of reaction was monitored by TLC. After completion of reaction, the reaction mixture was slowly quenched using ice-cold water (25 mL) and the product was extracted with EtOAc (2×75 mL). The combined organic layers were washed with water (50 mL), dried over sodium sulfate and concentrated to obtain a crude product which was purified by combiflash chromatography (reverse phase) to afford 2-(4-amino-3-(3-fluoro-5-hydroxyphenyl)-1H-pyrazolo[3,4-d]pyrimidin-1-yl)-5-fluoro-2,3-dihydro-1H-inden-1-one (82 mg) off-white solid. The crude product (12 mg) was added to ethanolic HCl (2 mL) and stirred for 30 min at RT. The reaction mixture was then concentrated under reduced pressure and lyophilized to afford 2-(4-amino-3-(3-fluoro-5-hydroxyphenyl)-1H-pyrazolo[3,4-d]pyrimidin-1-yl)-5-fluoro-2,3-dihydro-1H-inden-1-one (12.5 mg) as the HCl salt off-white solid. ¹HNMR (400 MHz, DMSO-d6) δ (ppm): 8.58 (s, 1H), 8.39 (d, J=2.6 Hz, 1H), 7.91 (dd, J=8.6, 2.6 Hz, 1H), 7.33-7.24 (m, 2H), 7.21 (dt, J=5.7, 3.7 Hz, 2H), 6.98 (d, J=8.6 Hz, 1H), 5.79 (q, J=7.5 Hz, 1H), 3.92 (s, 3H), 3.60-3.37 (m, 4H). Separation by chiral HPLC affords Compound Nos. 51a and 51b.

### Example 52: Preparation of Compound No. 52

### Synthesis of 4-[4-amino-1-(2,3-dihydro-1H-inden-2-yl)-1H-pyrazolo[3,4-d]pyrimidin-3-yl]pyridin-2-ol

Steps 1-5 are same as in Example 3.

### Step-6: Synthesis of 4-(4-amino-1-(2,3-dihydro-M-inden-2-yl)-1H-pyrazolo[3,4-d]pyrimidin-3-yl)pyridin-2-01

To 1-(2,3-dihydro-1H-inden-2-yl)-3-(2-methoxypyridin-4-yl)-1H-pyrazolo[3,4-d]pyrimidin-4-amine (55 mg, 0.153 mmol) was added ethanolic HCl (5 mL) and the resultant reaction mixture was heated at 60° C. for 24 h. The progress of reaction was monitored by LCMS. After completion of reaction, the reaction mixture was concentrated under reduced pressure, the residue obtained was basified using saturated NaHCO₃ solution (30 mL) and the product was extracted with EtOAc (50 mL). The organic layer was washed with water (25 mL), brine (25 mL), dried over sodium sulfate and concentrated to get a crude product which was purified by reverse phase combiflash chromatography affording 4-(4-amino-1-(2,3-dihydro-1H-inden-2-yl)-1H-pyrazolo[3,4-d]pyrimidin-3-yl)pyridin-2-ol (8.2 mg) as an off-white solid. The product was added to ethanolic HCl (2 mL) and stirred for 30 min at RT. The reaction mixture was then concentrated under reduced pressure and lyophilized to afford 4-(4-amino-1-(2,3-dihydro-1H-inden-2-yl)-1H-pyrazolo[3,4-d]pyrimidin-3-yl)pyridin-2-ol as the HCl salt (8.4 mg) as a light brown solid. ¹HNMR (400 MHz, Methanol-d4) δ (ppm): 8.41-8.30 (m, 1H), 7.86 (dd, J=9.4, 2.5 Hz, 1H), 7.80-7.69 (m, 1H), 7.28-7.23 (m, 2H), 7.24-7.15 (m, 2H), 6.67 (d, J=9.4 Hz, 1H), 5.82 (dd, J=9.0, 6.6 Hz, 1H), 3.59 (dd, J=15.9, 7.6 Hz, 2H), 3.49 (dd, J=15.9, 8.1 Hz, 2H).

### Example 53: Preparation of Compound No. 53

### Synthesis of {2-[4-amino-1-(2,3-dihydro-1H-inden-2-yl)-1H-pyrazolo[3,4-d]pyrimidin-3-yl]phenyl}methanol

To a stirred solution of 1-indan-2-yl-3-iodo-pyrazolo[3,4-d]pyrimidin-4-amine (100 mg, 0.265 mmol) and [2-(hydroxymethyl)phenyl]boronic acid (60 mg, 0.397 mmol) in DMF (3 mL) was added a solution of sodium carbonate (84.25 mg, 0.795 mmol) in water (3 mL) followed by addition of tetrakis(triphenylphosphine)palladium(0) (30.62 mg, 0.0265 mmol). The reaction mixture was allowed to heat at 80° C. overnight. The progress of reaction was monitored by TLC. After completion of reaction, the reaction mixture was concentrated under reduced pressure, washed with water (20 mL), and extracted with EtOAc (3×15 mL). The combined organic layer was dried over anhydrous sodium sulfate, concentrated under reduced pressure to give a crude product which was purified over reverse phase HPLC to afford [2-(4-amino-1-indan-2-yl-pyrazolo[3,4-d]pyrimidin-3-yl)phenyl]methanol (15 mg). The product was allowed to stir for 2 h in ethanolic HCl (2 mL) to give [2-(4-amino-1-indan-2-yl-pyrazolo[3,4-d]pyrimidin-3-yl)phenyl]methanol as the HCl salt. ¹H NMR (400 MHz, DMSO-d6) δ (ppm): 8.41-8.33 (m, 1H), 7.60 (d, J=7.9 Hz, 2H), 7.47 (d, J=7.9 Hz, 2H), 7.24 (ddt, J=23.9, 5.5, 3.2 Hz, 5H), 5.76 (p, J=8.0 Hz, 1H), 4.58 (s, 2H), 3.51 (s, 4H).

### Example 54: Preparation of Compound No. 54

### Synthesis of {3-[4-amino-1-(2,3-dihydro-1H-inden-2-yl)-1H-pyrazolo[3,4-d]pyrimidin-3-yl]phenyl}methanol

To a stirred solution of 1-indan-2-yl-3-iodo-pyrazolo[3,4-d]pyrimidin-4-amine (100 mg, 0.265 mmol) and [3-(hydroxymethyl)phenyl]boronic acid (60 mg, 0.397 mmol) in DMF (3 mL) was added a solution of sodium carbonate (84.25 mg, 0.795 mmol) in water (3 mL) followed by addition of tetrakis(triphenylphosphine)palladium(0) (30.62 mg, 0.0265 mmol). The reaction mixture was allowed to heat at 80° C. overnight. The progress of reaction was monitored by TLC. After completion of reaction, the reaction mixture was concentrated under reduced pressure, washed with water (20 mL), the product was extracted with EtOAc (3×15 mL). The combined organic layer was dried over anhydrous sodium sulfate, concentrated under reduced pressure to give a crude which was further purified over reverse phase HPLC to afford [3-(4-amino-1-indan-2-yl-pyrazolo[3,4-d]pyrimidin-3-yl)phenyl]methanol (15 mg). The compound was allowed to stir for 2 h in Ethanolic HCl (2 mL) to give required [3-(4-amino-1-indan-2-yl-pyrazolo[3,4-d]pyrimidin-3-yl)phenyl]methanol as the HCl salt. ¹HNMR (400 MHz, DMSO-d6) δ: 8.36 (s, 1H), 7.60 (s, 1H), 7.56-7.44 (m, 2H), 7.42 (d, J=6.6 Hz, 2H), 7.28 (dd, J=5.4, 3.4 Hz, 1H), 7.21 (dt, J=5.6, 3.2 Hz, 1H), 7.10 (s, 1H), 6.97 (s, 1H), 5.77 (p, J=7.9 Hz, 1H), 4.58 (s, 2H), 3.50 (d, J=7.9 Hz, 4H).

### Example 55: Preparation of Compound No. 55

### Synthesis of {4-[4-amino-1-(2,3-dihydro-1H-inden-2-yl)-1H-pyrazolo[3,4-d]pyrimidin-3-yl]phenyl}methanol

To a stirred solution of 1-indan-2-yl-3-iodo-pyrazolo[3,4-d]pyrimidin-4-amine (100 mg, 0.265 mmol) and [4-(hydroxymethyl)phenyl]boronic acid (60 mg, 0.397 mmol) in DMF (3 mL) was added a solution of sodium carbonate (84.25 mg, 0.795 mmol) in water (3 mL) followed by addition of tetrakis(triphenylphosphine)palladium(0) (30.62 mg, 0.0265 mmol). The reaction mixture was allowed to heat at 80° C. overnight. The progress of reaction was monitored by TLC. After the completion of reaction, the reaction mixture was concentrated under reduced pressure, washed with water (20 mL) and the product was extracted with EtOAc (3×15 mL). The combined organic layer was dried over anhydrous sodium sulfate, concentrated under reduced pressure to give a crude product which was purified over Reverse phase HPLC to afford [4-(4-amino-1-indan-2-yl-pyrazolo[3,4-d]pyrimidin-3-yl)phenyl]methanol (3.5 mg). The compound was allowed to stir for 2 h in Ethanolic HCl (2 mL) to give [4-(4-amino-1-indan-2-yl-pyrazolo[3,4-d]pyrimidin-3-yl)phenyl]methanol as the HCl salt. ¹HNMR (400 MHz, DMSO-d6) δ: 8.32 (s, 1H), 7.63 (d, J=7.7 Hz, 1H), 7.48 (td, J=7.3, 1.9 Hz, 1H), 7.42-7.31 (m, 2H), 7.24 (dt, J=7.2, 3.7 Hz, 2H), 7.21-7.11 (m, 3H), 5.74 (p, J=7.6 Hz, 1H), 4.41 (s, 2H) 3.51 (s, 4H).

### Example 56: Preparation of Compound No. 56

### Synthesis of 5-[4-amino-1-(2,3-dihydro-1H-inden-2-yl)-1H-pyrazolo[3,4-d]pyrimidin-3-yl]-2-chlorophenol

Steps 1-5 are same as in Example 3.

### Step-6: Synthesis of 4-chloro-3-methoxyphenylboronic acid

To a stirred solution of 4-bromo-1-chloro-2-methoxybenzene (2 g, 9.03 mmol) in diethyl ether (20 mL) was added n-BuLi (2 M in hexane; 5.2 mL, 10.4 mmol) at −78° C. dropwise. The reaction was allowed to stir at −78° C. for 30 min, and then triisopropyl borate (2.4 mL, 10.40 mmol) was slowly added to the reaction mixture at −78° C. The temperature of the reaction mixture was gradually raised to RT over a period of 1 h, and then the reaction was stirred at RT for 30 min. After 30 min, 3 N HCl (100 mL) was slowly added to the reaction mixture at 0° C. and the reaction was stirred for further 1 h. The progress of reaction was monitored by TLC. After completion of reaction, the reaction mixture was extracted with EtOAc (2×200 mL). The combined organic layers were washed with brine (100 mL), dried over sodium sulfate and concentrated to obtain a crude product. The residue obtained was triturated with hexane (2×25 mL) to afford 4-chloro-3-methoxyphenylboronic acid (900 mg) as a white solid.

### Step-7: Synthesis of 4-chloro-3-hydroxyphenylboronic acid

To a stirred suspension of 4-chloro-3-methoxyphenylboronic acid (900 mg, 4.82 mmol) in DCM (20 mL) was added BBr₃ (1 M in DCM; 19.3 mL, 19.30 mmol) at 0° C. dropwise and the resultant reaction mixture was stirred at RT for 16 h. The progress of reaction was monitored by TLC. After completion of reaction, the reaction mixture was slowly quenched using 3 N HCl (100 mL) and the product was extracted with EtOAc (2×200 mL). The organic layer was washed with water (100 mL), dried over sodium sulfate and concentrated to get a crude product which was washed with pentane (2×20 mL) to afford 4-chloro-3-hydroxyphenylboronic acid (780 mg) as an off-white solid, which was taken to the next step without further purification.

### Step-8: Synthesis of 5-(4-amino-1-(2,3-dihydro-M-inden-2-yl)-1H-pyrazolo[3,4-d]pyrimidin-3-yl)-2-chlorophenol

To a solution of 1-(2,3-dihydro-1H-inden-2-yl)-3-iodo-1H-pyrazolo[3,4-d]pyrimidin-4-amine (200 mg, 0.530 mmol) in DMF (5 mL) was added 4-chloro-3-hydroxyphenylboronic acid (119 mg, 0.690 mmol) at RT. Then, Na₂CO₃ (168 mg, 1.58 mmol) dissolved in water (5 mL) was added to the reaction mixture followed by addition of Pd(PPh₃)₄ (61 mg, 0.052 mmol) at RT and the resultant reaction mixture was heated at 80° C. for 2 h. The progress of reaction was monitored by LCMS. After completion of reaction, the reaction mixture was diluted with water (50 mL) and the product was extracted with EtOAc (2×100 mL). The combined organic layers were washed with water (2×100 mL), brine (50 mL), dried over sodium sulfate and concentrated to obtain a crude product which was purified by preparative HPLC affording 5-(4-amino-1-(2,3-dihydro-1H-inden-2-yl)-1H-pyrazolo[3,4-d]pyrimidin-3-yl)-2-chlorophenol (20 mg) as a white solid. To this was added ethanolic HCl (2 mL) and stirred for 30 min at RT. The reaction mixture was then concentrated under reduced pressure and lyophilized to afford of 5-(4-amino-1-(2,3-dihydro-1H-inden-2-yl)-1H-pyrazolo[3,4-d]pyrimidin-3-yl)-2-chlorophenol as the HCl salt (22 mg) white solid. ¹HNMR (400 MHz, DMSO-d6) δ (ppm): 10.49 (s, 1H), 8.36 (s, 1H), 7.47 (d, J=8.1 Hz, 1H), 7.27-7.15 (m, 5H), 7.05 (dd, J=8.2, 2.1 Hz, 1H), 5.75 (p, J=7.7 Hz, 1H), 3.5 (m, 4H).

### Example 57: Preparation of Compound Nos. 57, 57a and 57b

### Synthesis of 3-[4-amino-1-(5-fluoro-2,3-dihydro-1H-inden-2-yl)-1H-pyrazolo[3,4-d]pyrimidin-3-yl]-5-fluorophenol

To 2-(4-amino-3-(3-fluoro-5-hydroxyphenyl)-1H-pyrazolo[3,4-d]pyrimidin-1-yl)-5-fluoro-2,3-dihydro-1H-inden-1-one (70 mg, 0.177 mmol) was added TFA (2 mL) at 0° C. slowly followed by addition of triethylsilane (1.13 mL, 7.11 mmol). The resultant reaction mixture was heated at 100° C. for 12 h. The progress of reaction was monitored by LCMS. After completion of reaction, the reaction mixture was cooled to RT and concentrated under reduced pressure. The residue obtained was basified using saturated NaHCO₃ solution (25 mL) and the product was extracted with EtOAc (2×50 mL). The combined organic layers were washed with water (30 mL), brine (20 mL), dried over sodium sulfate and concentrated to get a crude product which was purified by combiflash chromatography (reverse phase) to afford 3-(4-amino-1-(5-fluoro-2,3-dihydro-1H-inden-2-yl)-1H-pyrazolo[3,4-d]pyrimidin-3-yl)-5-fluorophenol (9.8 mg) as an off-white solid. To this purified product was added ethanolic HCl (2 mL) and stirred for 30 min at RT. The reaction mixture was then concentrated under reduced pressure and lyophilized to afford 3-(4-amino-1-(5-fluoro-2,3-dihydro-1H-inden-2-yl)-1H-pyrazolo[3,4-d]pyrimidin-3-yl)-5-fluorophenol as the HCl salt (10 mg) as an off-white solid. ¹HNMR (400 MHz, Methanol-d4) δ (ppm): 8.43 (s, 1H), 7.25 (dd, J=8.4, 5.2 Hz, 1H), 7.06-6.93 (m, 1H), 6.94-6.83 (m, 3H), 6.69 (dt, J=10.8, 2.3 Hz, 1H), 5.90 (p, J=7.8 Hz, 1H), 3.58 (ddd, J=29.4, 15.2, 7.4 Hz, 4H). Separation by chiral HPLC affords Compound Nos. 57a and 57b.

### Example 58: Preparation of Compound No. 58

### Synthesis of (2E)-3-[4-amino-1-(2,3-dihydro-1H-inden-2-yl)-1H-pyrazolo[3,4-d]pyrimidin-3-yl]prop-2-enamide

Steps 1-5 are same as in Example 3.

### Step-6: Synthesis of (E)-ethyl 3-(4-amino-1-(2,3-dihydro-1H-inden-2-yl)-1H-pyrazolo[3,4-d]pyrimidin-3-yl)acrylate

To a stirred solution of 1-(2,3-dihydro-1H-inden-2-yl)-3-iodo-1H-pyrazolo[3,4-d]pyrimidin-4-amine (600 mg, 1.59 mmol) in 1, 4-dioxane (12 mL) were added ethyl acrylate (1.01 mL, 9.54 mmol) and triethylamine (1.55 mL, 11.13 mmol) at RT and the resultant reaction mixture was degassed under N2 for 15 min. Then, triphenylphosphine (208.61 mg, 0.795 mmol) and Pd(OAc)₂ (107.13 mg, 0.477 mmol) were successively added to the reaction mixture and the reaction mixture was stirred at 100° C. for 2.5 h. The progress of reaction was monitored by TLC. After completion of reaction, the reaction mixture was diluted with water (100 mL) and the product was extracted with EtOAc (2×200 mL). The combined organic layers were washed with water (100 mL) then with brine (60 mL), dried over Na₂SO₄ and concentrated to obtain a crude product which was triturated with acetone-hexane (1:3) to afford (E)-ethyl 3-(4-amino-1-(2,3-dihydro-1H-inden-2-yl)-1H-pyrazolo[3,4-d]pyrimidin-3-yl)acrylate (520 mg) as a yellow solid.

### Step-7: Synthesis of (E)-3-(4-amino-1-(2,3-dihydro-1H-inden-2-yl)-1H-pyrazolo[3,4-d]pyrimidin-3-yl)acrylic acid

To a stirred solution of (E)-ethyl 3-(4-amino-1-(2,3-dihydro-1H-inden-2-yl)-1H-pyrazolo[3,4-d]pyrimidin-3-yl)acrylate (400 mg, 1.14 mmol) in EtOH (8 mL) was added NaOH (183.1 mg, 4.57 mmol) dissolved in water (4 mL) and the resultant reaction mixture was heated at 80° C. for 2 h. The progress of reaction was monitored by TLC and LCMS. After completion of reaction, the reaction mixture was concentrated under reduced pressure, the residue obtained was dissolved in water (10 mL) and washed with diethyl ether (2×25 mL). The aq. layer was then acidified using 3 N HCl at 0° C. (pH=2-3) and then the product was extracted using EtOAc (3×250 mL). The combined organic layers were washed with brine (50 mL), dried over Na₂SO₄ and concentrated to obtain a crude product which was triturated with pentane-Et₂O (3:1) to afford (E)-3-(4-amino-1-(2,3-dihydro-1H-inden-2-yl)-1H-pyrazolo[3,4-d]pyrimidin-3-yl)acrylic acid (320 mg) as an off-white solid.

### Step-8: Synthesis of (E)-3-(4-amino-1-(2,3-dihydro-1H-inden-2-yl)-1H-pyrazolo[3,4-d]pyrimidin-3-yl)acrylamide

To a stirred solution of (E)-3-(4-amino-1-(2,3-dihydro-1H-inden-2-yl)-1H-pyrazolo[3,4-d]pyrimidin-3-yl)acrylic acid (250 mg, 0.778 mmol) in DMF (6 mL) were successively added EDC. HCl (223.7 mg, 1.16 mmol), HOBt (157.6 mg, 1.16 mmol), NH₄Cl (249.6 mg, 4.66 mmol) and N-methylmorpholine (0.128 mL, 1.16 mmol) and the resultant reaction mixture was stirred at RT for 5 h. The progress of reaction was monitored by TLC and LCMS. After completion of reaction, the reaction mixture was diluted with water (50 mL) and the product was extracted with EtOAc (2×100 mL). The combined organic layers were washed with brine (50 mL), dried over Na₂SO₄ and concentrated to obtain a crude product which was purified by combiflash chromatography (reverse phase) to afford (E)-3-(4-amino-1-(2,3-dihydro-1H-inden-2-yl)-1H-pyrazolo[3,4-d]pyrimidin-3-yl)acrylamide (7.9 mg) as an off-white solid. The purified product was added to ethanolic HCl (2 mL) and stirred for 30 min at RT. The reaction mixture was then concentrated under reduced pressure and lyophilized to afford (E)-3-(4-amino-1-(2,3-dihydro-1H-inden-2-yl)-1H-pyrazolo[3,4-d]pyrimidin-3-yl)acrylamide as the HCl salt (8 mg) as an off-white solid. ¹HNMR (400 MHz, Methanol-d4) δ (ppm): 8.34 (s, 1H), 7.81 (s, 1H), 7.29-7.15 (m, 4H), 6.96 (d, J=15.5 Hz, 1H), 5.83 (p, J=7.9 Hz, 1H), 3.55 (m, 4H).

### Example 59: Preparation of Compound No. 59

### Synthesis of N-{3-[4-amino-1-(2,3-dihydro-1H-inden-2-yl)-1H-pyrazolo[3,4-d]pyrimidin-3-yl]phenyl}acetamide

To a solution of 1-indan-2-yl-3-iodo-pyrazolo[3,4-d]pyrimidin-4-amine (300 mg, 0.79 mmol) and N-[3-(4,4,5,5-tetramethyl-1,3,2-dioxaborolan-2-yl)phenyl]acetamide (415 mg, 1.59 mmol) in N,N-dimethylformamide (3 mL) was added a solution of sodium carbonate (168 mg, 1.59 mmol) in water (3 mL) followed by the addition of tetrakis(triphenyl phosphine)palladium(0) (91 mg, 0.079 mmol). The reaction mixture was heated in a reagent bottle at 90° C. for 3 h. The progress of reaction was monitored by TLC. After completion of reaction, water (10 mL) was added to the reaction mixture and the product was extracted using EtOAc (2×50 mL). The combined organic layer was again washed with water (3×30 mL) and brine (30 mL). The organic layer was separated, dried over anhydrous sodium sulfate and concentrated to obtain a crude product which was purified by reverse phase preparative HPLC giving N-[3-(4-amino-1-indan-2-yl-pyrazolo[3,4-d]pyrimidin-3-yl)phenyl]acetamide as the formate salt (14.44 mg). ¹HNMR (400 MHz, DMSO-d6) δ (ppm): 10.17 (s, 1H), 8.27 (s, 1H), 7.89 (s, 1H), 7.59 (d, J=8.3 Hz, 1H), 7.44 (t, J=7.9 Hz, 1H), 7.35-7.24 (m, 3H), 7.24-7.09 (m, 2H), 6.29 (s, 1H), 5.74 (m, 1H), 3.49 (d, J=8.0 Hz, 4H), 2.06 (s, 3H).

### Example 60: Preparation of Compound No. 60

### Synthesis of N-{3-[4-amino-1-(2,3-dihydro-1H-inden-2-yl)-1H-pyrazolo[3,4-d]pyrimidin-3-yl]-5-fluorophenyl}acetamide

To a solution of 1-indan-2-yl-3-iodo-pyrazolo[3,4-d]pyrimidin-4-amine (125 mg, 0.33 mmol) and N-[3-fluoro-5-(4,4,5,5-tetramethyl-1,3,2-dioxaborolan-2-yl)phenyl]acetamide (185 mg, 0.66 mmol) in N,N-dimethylformamide (2 mL) was added a solution of sodium carbonate (70 mg, 0.66 mmol) in water (2 mL) followed by the addition of tetrakis(triphenylphosphine)palladium(0) (38 mg, 0.033 mmol). The reaction mixture was heated in a reagent bottle at 90° C. for 2 h. The reaction was monitored by TLC. After completion of reaction, water (10 mL) was added to the reaction mixture and the product was extracted using EtOAc (2×50 mL). The combined organic layer was again washed with water (3×30 mL) and brine (30 mL). The organic layer was separated, dried over anhydrous sodium sulfate and concentrated to obtain a crude product which was purified by reverse phase preparative HPLC giving the pure compound (23.1 mg) as the free base. The latter was dissolved in ethanolic HCl (2 mL) stirred for 30 min and then ethanolic HCl was evaporated under reduced pressure to give N-[3-(4-amino-1-indan-2-yl-pyrazolo[3,4-d]pyrimidin-3-yl)-5-fluoro-phenyl]acetamide as the HCl salt (24.36 mg). ¹HNMR (400 MHz, Methanol-d4) δ (ppm): 10.25 (s, 1H), 8.41 (s, 1H), 7.84-7.72 (s, 1H), 7.38 (m, 1H), 7.30-7.24 (m, 2H), 7.22-7.15 (m, 3H), 5.87 (m, 2H), 3.62 (dd, J=15.9, 7.5 Hz, 2H), 3.53 (dd, J=15.9, 8.4 Hz, 2H). 2.18 (s, 3H).

### Example 61: Preparation of Compound No. 61

### Synthesis of 1-(2,3-dihydro-1H-inden-2-yl)-3-(5-methoxypyridin-3-yl)-1H-pyrazolo[3,4-d]pyrimidin-4-amine

To a solution of 1-indan-2-yl-3-iodo-pyrazolo[3,4-d]pyrimidin-4-amine (150 mg, 0.39 mmol) and (5-methoxy-3-pyridyl)boronic acid (91 mg, 0.59 mmol) in N,N-dimethylformamide (3 mL) was added a solution of sodium carbonate (84 mg, 0.79 mmol) in water (3 mL) followed by the addition of tetrakis(triphenylphosphine)palladium(0) (45 mg, 0.039 mmol). The reaction mixture was heated in a reagent bottle at 70° C. overnight. The progress of reaction was monitored by TLC. After completion of reaction, water (10 mL) was added to the reaction mixture and the product was extracted using EtOAc (2×50 mL). The combined organic layer was again washed with water (3×30 mL) and brine (30 mL). The organic layer was separated, dried over anhydrous sodium sulfate and concentrated to obtain a crude product which was purified by reverse phase preparative HPLC giving pure compound (6.1 mg). The latter was dissolved in ethanolic HCl (2 mL) stirred for 30 min and then ethanolic HCl was evaporated under reduced pressure to give 1-indan-2-yl-3-(5-methoxy-3-pyridyl)pyrazolo[3,4-d]pyrimidin-4-amine as the HCl salt (6.22 mg). ¹HNMR (400 MHz, Methanol-d4) δ (ppm): 8.80-8.70 (m, 2H), 8.53 (s, 1H), 8.42-8.37 (s, 1H), 7.30-7.16 (m, 4H), 5.95 (m, 1H), 4.11 (s, 3H), 3.60 (d, J=7.5 Hz, 4H)

### Example 62: Preparation of Compound No. 62

### Synthesis of 7-(2,3-dihydro-1H-inden-2-yl)-5-(3-fluoro-5-methoxyphenyl)-7H-pyrrolo[2,3-d]pyrimidin-4-amine

### Step-1: Synthesis of 4-chloro-5-iodo-7H-pyrrolo[2,3-d]pyrimidine

4-Chloro-7H-pyrrolo[2,3-d]pyrimidine (2 g, 13.02 mmol) was charged in DCM (60 mL) and was added N-iodosuccinimide (3.80 g, 16.92 mmol). To this reaction mixture was added DMF (6 mL) and the reaction mixture was stirred at RT for 1 h. The progress of reaction was monitored by TLC and NMR. After completion of reaction, the solid obtained was filtered off and washed with pentane (30 mL). The solid obtained was dried under vacuum to get 4-chloro-5-iodo-7H-pyrrolo[2, 3-d]pyrimidine (2.8 g).

### Step-2: Synthesis of 2, 3-dihydro-1H-inden-2-yl methanesulfonate

Indan-2-ol (5 g, 37.26 mmol) was charged in DCM (150 mL). Triethylamine (25.77 mL, 186.2 mmol) was added in to reaction mixture and reaction mixture was cooled to 0° C. Mesyl chloride (7.25 mL, 93.15 mmol) was added dropwise into the reaction mixture and the reaction mixture was allowed to come to RT. The reaction mixture was stirred at RT for 60 min. The progress of reaction was monitored by TLC and NMR. After completion of reaction, ice-water (50 mL) was added in to the reaction mixture and the organic layer was separated. The organic layer was washed with water (3×30 mL), dried over anhydrous sodium sulfate and concentrated under reduced pressure to obtain a crude product which was purified by column chromatography on silica gel (100-200 mesh) using 0-20% EtOAc-hexane to get indan-2-yl methanesulfonate (6.5 g).

### Step-3: Synthesis of 4-chloro-7-(2,3-dihydro-1H-inden-2-yl)-5-iodo-7H-pyrrolo[2,3-d]pyrimidine

4-Chloro-5-iodo-7H-pyrrolo[2,3-d]pyrimidine (2.8 g, 10 mmol) was added in DMF (20 mL). Potassium carbonate (2.48 g, 18 mmol) followed by indan-2-yl methanesulfonate (2.33 g, 11 mmol) was added to the reaction mixture and it was heated to 80° C. The reaction mixture was stirred for 15 min at 80° C. and was monitored by TLC and NMR. After completion of reaction, the reaction mixture was allowed to come to RT and water (60 mL) was added. The solid obtained was filtered off and washed with acetone (20 mL) followed by the washing of pentane (30 mL) to get 630 mg of 4-chloro-7-(2,3-dihydro-1H-inden-2-yl)-5-iodo-7H-pyrrolo[2,3-d]pyrimidine. ¹HNMR (400 MHz, DMSO-d6): δ (ppm): 8.67 (s, 1H), 7.90 (s, 1H), 7.30 (dt, J=7.5, 3.7 Hz, 2H), 7.23 (dd, J=5.5, 3.3 Hz, 2H), 5.63 (p, J=7.4 Hz, 1H), 3.47 (m, 4H). LCMS (M+1): 396.1

### Step-4: Synthesis of 7-(2,3-dihydro-1H-inden-2-yl)-5-iodo-7H-pyrrolo[2,3-d]pyrimidin-4-amine

4-Chloro-7-(2,3-dihydro-1H-inden-2-yl)-5-iodo-7H-pyrrolo[2,3-d]pyrimidine (600 mg, 1.51 mmol) was placed in an autoclave. Liquor ammonia (40 mL) and THF (25 mL) was added to it. The reaction mixture was stirred at 150° C. for 16 h. The progress of reaction was monitored by TLC and LCMS. After completion of reaction, the reaction mixture was cooled to RT and solid obtained was filtered off. This solid was washed with pentane (30 mL) to give 7-(2,3-dihydro-1H-inden-2-yl)-5-iodo-7H-pyrrolo[2,3-d]pyrimidin-4-amine (380 mg).

### Step-5: Synthesis of 7-(2,3-dihydro-1H-inden-2-yl)-5-(3-fluoro-5-methoxyphenyl)-7H-pyrrolo[2,3-d]pyrimidin-4-amine

7-(2,3-Dihydro-1H-inden-2-yl)-5-iodo-7H-pyrrolo[2,3-d]pyrimidin-4-amine (150 mg, 0.398 mmol) and tetrakistriphenyl phosphine palladium (0) (45.9 mg, 0.039 mmol) were charged in DME (20 mL) and nitrogen was purged in to the reaction mixture for 5 min. Potassium carbonate (55 g, 0.398 mmol) and 3-fluoro-5-methoxyphenylboronic acid (169.4 mg, 0.99 mmol) were added to this reaction mixture. Water (3 mL) was added and again purged with nitrogen for 5 min. The reaction mixture was stirred at 100° C. for 18 h. The progress of reaction was monitored by TLC and LCMS. After completion of reaction, EtOAc (150 mL) was added and organic layer was separated. The organic layer was washed with water (3×20 mL) and dried over anhydrous sodium sulfate. EtOAc was evaporated under reduced pressure to obtain a crude product which was purified using reverse phase chromatography to get 7-(2,3-dihydro-1H-inden-2-yl)-5-(3-fluoro-5-methoxyphenyl)-7H-pyrrolo[2,3-d]pyrimidin-4-amine (25 mg). ¹HNMR (400 MHz, DMSO-d6): δ (ppm): 8.17 (s, 1H), 7.42 (s, 1H), 7.25 (m, 4H), 6.87-6.72 (m, 3H), 6.24 (s, broad, 2H), 5.56 (p, J=7.7 Hz, 1H), 3.80 (s, 3H), 3.40 (m, 4H). LCMS (M+1): 374.8.

### Example 63: Preparation of Compound No. 63

### Synthesis of 3-[4-amino-7-(2,3-dihydro-1H-inden-2-yl)-7H-pyrrolo[2,3-d]pyrimidin-5-yl]-5-fluorophenol

Steps 1 to 4 are same as in Example 62.

### Step-5: Synthesis of 7-(2,3-dihydro-1H-inden-2-yl)-5-(3-fluoro-5-hydroxyphenyl)-7H-pyrrolo[2,3-d]pyrimidin-4-amine

7-(2,3-Dihydro-1H-inden-2-yl)-5-iodo-7H-pyrrolo[2,3-d]pyrimidin-4-amine (100 mg, 0.265 mmol) and tetrakistriphenyl phosphine palladium (0) (30.62 mg, 0.026 mmol) were charged in DMF (1.5 mL) and nitrogen was purged in to the reaction mixture for 5 min. 3-fluoro-5-hydroxyphenylboronic acid (62.23 mg, 0.39 mmol) was added in to the reaction mixture. Then, sodium carbonate (64.6 mg, 0.60 mmol) dissolved in water (1.5 mL) was added to the reaction mixture and again purged with nitrogen for 5 min. The reaction mixture was stirred at 100° C. for 3 h. The progress of reaction was monitored by TLC and LCMS. After completion of reaction, the reaction mixture was allowed to come to RT and EtOAc (150 mL) was added and organic layer was separated. The organic layer was washed with water (3×20 mL) and dried over anhydrous sodium sulfate. EtOAc was evaporated under reduced pressure to obtain a crude product which was purified using reverse phase chromatography to get 7-(2,3-dihydro-1H-inden-2-yl)-5-(3-fluoro-5-hydroxyphenyl)-7H-pyrrolo[2,3-d]pyrimidin-4-amine (25 mg). ¹HNMR (400 MHz, DMSO-d6): δ (ppm): 10.02 (s, broad, 1H), 8.16 (s, 1H), 7.33-7.17 (m, 5H), 6.66 (dp, J=6.0, 1.6 Hz, 2H), 6.51 (dt, J=10.9, 2.3 Hz, 1H), 6.22 (s, broad, 2H), 5.56 (p, J=7.4 Hz, 1H), 3.40 (m, 4H). LCMS (M+1): 361.3.

### Example 64: Preparation of Compound No. 64

### Synthesis of (2E)-3-[4-amino-1-(2,3-dihydro-1H-inden-2-yl)-1H-pyrazolo[3,4-d]pyrimidin-3-yl]prop-2-en-1-ol

To LAH (39.12 mg, 1.03 mmol) in dry THF (3 mL) was added (E)-ethyl 3-(4-amino-1-(2,3-dihydro-1H-inden-2-yl)-1H-pyrazolo[3,4-d]pyrimidin-3-yl)acrylate (180 mg, 0.515 mmol) dissolved in THF (3 mL) at 0° C. slowly. The resultant reaction mixture was allowed to stir at 0° C. for 2 h. The progress of reaction was monitored by TLC. After completion of reaction, the reaction mixture was slowly quenched using saturated Na₂SO₄ solution at 0° C. slowly. The reaction mixture was then filtered using a celite bed. The filtrate obtained (EtOAc and THF) was washed with water (50 mL), brine (40 mL), dried over Na₂SO₄ and concentrated to obtain a crude product which was purified first by preparative TLC and then using reverse phase combiflash chromatography to afford 3-(4-amino-1-(2,3-dihydro-1H-inden-2-yl)-1H-pyrazolo[3,4-d]pyrimidin-3-yl)propan-1-ol (Peak 1: 6 mg) and (E)-3-(4-amino-1-(2,3-dihydro-1H-inden-2-yl)-1H-pyrazolo[3,4-d]pyrimidin-3-yl)prop-2-en-1-ol (Peak 2: 3 mg) as the HCl salt. ¹HNMR (400 MHz, Methanol-d4) δ (ppm): 8.33 (s, 1H), 7.30-7.12 (m, 4H), 5.77 (p, J=7.9 Hz, 1H), 3.64 (t, J=5.9 Hz, 2H), 3.55-3.42 (m, 4H), 3.09 (t, J=7.3 Hz, 2H), 1.95 (p, J=6.6 Hz, 2H), 1.37-1.22 (m, 1H).

### Example 65: Preparation of Compound No. 65

### Synthesis of 3-[4-amino-1-(2,3-dihydro-1H-inden-2-yl)-1H-pyrazolo[3,4-d]pyrimidin-3-yl]propan-1-ol

To LAH (39.12 mg, 1.03 mmol) in dry THF (3 mL) was added (E)-ethyl 3-(4-amino-1-(2,3-dihydro-1H-inden-2-yl)-1H-pyrazolo[3,4-d]pyrimidin-3-yl)acrylate (180 mg, 0.515 mmol) dissolved in THF (3 mL) at 0° C. slowly. The resultant reaction mixture was allowed to stir at 0° C. for 2 h. The progress of reaction was monitored by TLC. After completion of reaction, the reaction mixture was slowly quenched using saturated Na₂SO₄ solution at 0° C. slowly. The reaction mixture was then filtered using celite bed. The filtrate obtained (EtOAc and THF) was washed with water (50 mL), brine (40 mL), dried over Na₂SO₄ and concentrated to obtain a crude product which was purified first by preparative TLC and then using reverse phase combiflash chromatography to afford 3-(4-amino-1-(2,3-dihydro-1H-inden-2-yl)-1H-pyrazolo[3,4-d]pyrimidin-3-yl)propan-1-ol (Peak 1: 6 mg) and (E)-3-(4-amino-1-(2,3-dihydro-1H-inden-2-yl)-1H-pyrazolo[3,4-d]pyrimidin-3-yl)prop-2-en-1-ol (Peak 2: 3 mg) as the HCl salt. ¹HNMR (400 MHz, Methanol-d4) δ (ppm): 8.34 (s, 1H), 7.31-7.12 (m, 4H), 6.97 (d, J=15.7 Hz, 1H), 6.74 (dt, J=15.7, 4.8 Hz, 1H), 5.80 (p, J=7.8 Hz, 1H), 4.35-4.25 (m, 2H), 3.59-3.39 (m, 4H).

### Example 66: Preparation of Compound No. 66

### Synthesis of 3-(4-amino-3-methoxyphenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and 2-methoxy-4-(4,4,5,5-tetramethyl-1,3,2-dioxaborolan-2-yl)aniline (297 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(4-amino-3-methoxy-phenyl)-1-indan-2-yl-pyrazolo[3,4-d]pyrimidin-4-amine (120 mg) as an off-white solid, which was dissolved in 10 mL ethanolic HCl and concentrated under reduced pressure to obtain 3-(4-amino-3-methoxy-phenyl)-1-indan-2-yl-pyrazolo[3,4-d]pyrimidin-4-amine hydrochloride salt (120 mg) as a light black solid. ¹HNMR (400 MHz, Methanol-d4) δ (ppm): 8.46 (s, 1H), 7.46 (dd, J=5.0, 3.1 Hz, 2H), 7.35 (dd, J=8.0, 1.7 Hz, 1H), 7.26 (dt, J=7.3, 3.7 Hz, 2H), 7.25-7.17 (m, 2H), 5.90 (p, J=7.9 Hz, 1H), 4.03 (s, 3H), 3.62 (dd, J=16.0, 7.5 Hz, 2H), 3.54 (dd, J=16.0, 8.3 Hz, 2H). LCMS: 373 (M+1).

### Example 67: Preparation of Compound No. 67

### Synthesis of 3-[4-amino-1-(2,3-dihydro-1H-inden-2-yl)-1H-pyrazolo[3,4-d]pyrimidin-3-yl]-5-chlorophenol

To a solution of 1-indan-2-yl-3-iodo-pyrazolo[3,4-d]pyrimidin-4-amine (300 mg, 0.795 mmol) and 3-chloro-5-(4,4,5,5-tetramethyl-1,3,2-dioxaborolan-2-yl)phenol (304 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(4-amino-1-indan-2-yl-pyrazolo[3,4-d]pyrimidin-3-yl)-5-chloro-phenol (175 mg) as an off-white solid, which was dissolved in 10 mL ethanolic HCl and concentrated under reduced pressure to 3-(4-amino-1-indan-2-yl-pyrazolo[3,4-d]pyrimidin-3-yl)-5-chloro-phenol (177 mg) off-white solid. NMR (400 MHz, Methanol-d4) δ (ppm): 8.42 (s, 1H), 7.31-7.24 (m, 2H), 7.21 (dd, J=5.6, 3.3 Hz, 2H), 7.14 (t, J=1.7 Hz, 1H), 6.99 (dt, J=17.0, 2.0 Hz, 2H), 5.86 (p, J=8.2 Hz, 1H), 3.62 (dd, J=15.9, 7.5 Hz, 2H), 3.53 (dd, J=16.0, 8.3 Hz, 2H). LCMS: 378 (M+1).

### Example 68: Preparation of Compound No. 68

### Synthesis of 5-[4-amino-1-(2,3-dihydro-1H-inden-2-yl)-1H-pyrazolo[3,4-d]pyrimidin-3-yl]-2-methoxyphenol

To a solution of 1-indan-2-yl-3-iodo-pyrazolo[3,4-d]pyrimidin-4-amine (300 mg, 0.795 mmol) and 2-methoxy-5-(4,4,5,5-tetramethyl-1,3,2-dioxaborolan-2-yl)phenol (298 mg, 1.193 mmol) in DMF (5 mL) was added a solution of sodium carbonate (253 mg, 2.387 mmol) in water (5 mL) followed by the addition of tetrakis(triphenylphosphine) palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2-methoxy-phenol (115 mg) as an off-white solid, which was dissolved in 10 mL ethanolic HCl and concentrated under reduced pressure to Synthesis of 5-(4-amino-1-indan-2-yl-pyrazolo[3,4-d]pyrimidin-3-yl)-2-methoxy-phenol hydrochloride salt (117 mg) off-white solid. ¹HNMR (400 MHz, DMSO-d6) δ (ppm): 9.32 (s, 1H), 8.32 (s, 1H), 7.27 (dt, J=7.2, 3.7 Hz, 2H), 7.26-7.16 (m, 2H), 7.12-6.92 (m, 3H), 5.78-5.64 (m, 1H), 3.82 (s, 3H), 3.48 (d, J=7.9 Hz, 4H). LCMS: 374 (M+1).

### Example 69: Preparation of Compound No. 69

### Synthesis of 1-(2,3-dihydro-1H-inden-2-yl)-3-[3-methoxy-4-(trifluoromethyl)phenyl]-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and [3-methoxy-4-(trifluoromethyl)phenyl]boronic acid (291 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 1-indan-2-yl-3-[3-methoxy-4-(trifluoromethyl)phenyl]pyrazolo[3,4-d]pyrimidin-4-amine (121 mg) as an off-white solid. To obtain compound (71 mg) was dissolved in 10 mL ethanolic HCl and concentrated under reduced pressure to 1-indan-2-yl-3-[3-methoxy-4-(trifluoromethyl) phenyl]pyrazolo[3,4-d]pyrimidin-4-amine (71.5 mg) as a white solid. ¹HNMR (400 MHz, DMSO-d6) δ (ppm): 8.39 (d, J=11.0 Hz, 1H), 7.73 (dd, J=8.3, 4.4 Hz, 1H), 7.44 (d, J=4.0 Hz, 1H), 7.40-7.33 (m, 1H), 7.28 (q, J=4.2 Hz, 2H), 7.21 (q, J=4.4 Hz, 2H), 5.85-5.67 (m, 1H), 3.94 (s, 3H), 3.51 (dd, J=8.2, 4.3 Hz, 4H). LCMS: 426 (M+1).

### Example 70: Preparation of Compound No. 70

### Synthesis of 1-(2,3-dihydro-1H-inden-2-yl)-3-{1H-pyrrolo[2,3-b]pyridin-4-yl}-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and 4-(4,4,5,5-tetramethyl-1,3,2-dioxaborolan-2-yl)-1H-pyrrolo[2,3-b]pyridine (291 mg, 1.193 mmol) in DMF (5 mL) was added a solution of sodium carbonate (253 mg, 2.387 mmol) in water (5 mL) followed by the addition of tetrakis(triphenylphosphine) palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 1-indan-2-yl-3-(1H-pyrrolo[2,3-b]pyridin-4-yl)pyrazolo[3,4-d]pyrimidin-4-amine (28.5 mg) as an off-white solid, which was dissolved in 10 mL ethanolic HCl and concentrated under reduced pressure to 1-indan-2-yl-3-(1H-pyrrolo[2,3-b]pyridin-4-yl)pyrazolo[3,4-d]pyrimidin-4-amine (28.5 mg) as an off-white solid. ¹HNMR (400 MHz, DMSO-d6) δ (ppm): 12.10 (s, 1H), 8.55 (s, 1H), 8.39 (s, 1H), 7.59 (d, J=3.2 Hz, 1H), 7.30 (d, J=7.2 Hz, 4H), 7.21 (s, 2H), 6.47 (s, 1H), 5.89-5.81 (m, 1H), 3.51 (dd, J=8.2, 4.3 Hz, 4H). LCMS: 368 (M+1).

### Example 71: Preparation of Compound No. 71

### Synthesis of 1-(2,3-dihydro-1H-inden-2-yl)-3-(4-ethoxy-3-methoxyphenyl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 4-ethoxy-3-methoxyphenylboronic acid (233.8 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2.5 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 155 mg of 1-(2,3-dihydro-1H-inden-2-yl)-3-(4-ethoxy-3-methoxyphenyl)-1H-pyrazolo[3,4-d]pyrimidin-4-amine as a light brown solid. To this was added ethanolic HCl (10 mL) and stirred for 30 min at RT. The reaction mixture was then concentrated under reduced pressure and lyophilized to afford 1-(2,3-dihydro-1H-inden-2-yl)-3-(4-ethoxy-3-methoxyphenyl)-1H-pyrazolo[3,4-d]pyrimidin-4-amine (159 mg) as the HCl salt (light brown solid). ¹HNMR (400 MHz, DMSO-d6) δ (ppm): 8.47 (s, 1H), 7.37-6.90 (m, 7H), 5.77 (p, J=7.9 Hz, 1H), 4.07 (q, J=7.0 Hz, 2H), 3.50-3.35 (m, 4H), 3.80 (s, 3H), 1.44-1.21 (m, 3H).

### Example 72: Preparation of Compound No. 72

### Synthesis of 3-[4-amino-1-(2,3-dihydro-1H-inden-2-yl)-1H-pyrazolo[3,4-d]pyrimidin-3-yl]-5-(trifluoromethoxy)phenol

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3-hydroxy-5-(trifluoromethoxy) phenylboronic acid (264.7 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2.5 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 112 mg of 3-(4-amino-1-(2,3-dihydro-1H-inden-2-yl)-1H-pyrazolo[3,4-d]pyrimidin-3-yl)-5-(trifluoromethoxy)phenol as an off-white solid. To this was added ethanolic HCl (10 mL) and stirred for 30 min at RT. The reaction mixture was then concentrated under reduced pressure and lyophilized to afford 3-(4-amino-1-(2,3-dihydro-1H-inden-2-yl)-1H-pyrazolo[3,4-d]pyrimidin-3-yl)-5-(trifluoromethoxy)phenol (114 mg) as the HCl salt (off-white solid). ¹HNMR (400 MHz, DMSO-d6) δ (ppm): 10.41 (s, 1H), 8.42 (s, 1H), 7.35-7.11 (m, 5H), 7.01 (d, J=26.5 Hz, 1H), 6.82 (s, 1H), 5.76 (p, J=7.7 Hz, 1H), 1.38 (s, 1H), 3.5-3.3 (m, 4H).

### Example 73: Preparation of Compound No. 73

### Synthesis of 3-(3-amino-4-methoxyphenyl)-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3-amino-4-methoxyphenylboronic acid (199.2 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2.5 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 60 mg of 3-(3-amino-4-methoxyphenyl)-1-(2,3-dihydro-1H-inden-2-yl)-1H-pyrazolo[3,4-d]pyrimidin-4-amine as an off-white solid. To this was added ethanolic HCl (10 mL) and stirred for 30 min at RT. The reaction mixture was then concentrated under reduced pressure and lyophilized to afford 3-(3-amino-4-methoxyphenyl)-1-(2,3-dihydro-1H-inden-2-yl)-1H-pyrazolo[3,4-d]pyrimidin-4-amine (60.5 mg) as the HCl salt (off-white solid). ¹HNMR (400 MHz, DMSO-d6) δ (ppm): 8.51 (m, 3H), 7.39 (d, J=5.7 Hz, 1H), 7.28 (t, J=4.3 Hz, 3H), 7.21 (d, J=5.3 Hz, 3H), 5.84-5.68 (m, 1H), 3.92 (s, 3H), 3.52-3.4 (m, 4H).

### Example 74: Preparation of Compound No. 74

### Synthesis of 1-(2,3-dihydro-1H-inden-2-yl)-3-(3,4-dihydro-2H-1,5-benzodioxepin-7-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3,4-dihydro-2H-benzo[b][1,4]dioxepin-7-ylboronic acid (231.4 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2.5 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 73 mg of 1-(2,3-dihydro-1H-inden-2-yl)-3-(3,4-dihydro-2H-benzo[b][1,4]dioxepin-7-yl)-1H-pyrazolo[3,4-d]pyrimidin-4-amine as an off-white solid. To this was added ethanolic HCl (10 mL) and stirred for 30 min at RT. The reaction mixture was then concentrated under reduced pressure and lyophilized to afford 1-(2,3-dihydro-1H-inden-2-yl)-3-(3,4-dihydro-2H-benzo[b][1,4]dioxepin-7-yl)-1H-pyrazolo[3,4-d]pyrimidin-4-amine (75 mg) as the HCl salt (off-white solid). ¹HNMR (400 MHz, DMSO-d6) δ (ppm): 8.42 (s, 1H), 7.27 (dt, J=7.4, 3.6 Hz, 2H), 7.22-7.18 (m, 4H), 7.10 (d, J=8.8 Hz, 1H), 5.74 (d, J=7.7 Hz, 1H), 4.19 (t, J=5.4 Hz, 4H), 3.48-3.35 (m, 4H), 2.14 (p, J=5.5 Hz, 2H).

### Example 75: Preparation of Compound No. 75

### Synthesis of 3-(2,2-difluoro-2H-1,3-benzodioxol-5-yl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (2,2-difluoro-1,3-benzodioxol-5-yl)boronic acid (241 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(2,2-difluoro-1,3-benzodioxol-5-yl)-1-indan-2-yl-pyrazolo[3,4-d]pyrimidin-4-amine (150 mg) as an off-white solid, dissolved in 10 mL ethanolic HCl and concentrated under reduced pressure to obtain 3-(2,2-difluoro-1,3-benzodioxol-5-yl)-1-indan-2-yl-pyrazolo[3,4-d]pyrimidin-4-amine hydrochloride salt (150 mg) as a light brown solid. ¹HNMR (400 MHz, DMSO-d6) δ (ppm): 8.41 (s, 1H), 7.60 (d, J=1.6 Hz, 1H), 7.53 (d, J=8.3 Hz, 1H), 7.43 (dd, J=8.3, 1.6 Hz, 1H), 7.26 (dt, J=7.6, 3.7 Hz, 2H), 7.20 (dd, J=5.5, 3.2 Hz, 2H), 5.76 (p, J=7.8 Hz, 1H), 3.51 (m, 4H). LCMS: 408 (M+1).

### Example 76: Preparation of Compound No. 76

### Synthesis of 1-(2,3-dihydro-1H-inden-2-yl)-3-[3-ethoxy-4-(trifluoromethoxy)phenyl]-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and [3-ethoxy-4-(trifluoromethoxy)phenyl]boronic acid (299 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-[3-ethoxy-4-(trifluoromethoxy)phenyl]-1-indan-2-yl-pyrazolo[3,4-d]pyrimidin-4-amine (165 mg) as an off-white solid, dissolved in 10 mL ethanolic HCl and concentrated under reduced pressure to obtain 3-[3-ethoxy-4-(trifluoromethoxy)phenyl]-1-indan-2-yl-pyrazolo[3,4-d]pyrimidin-4-amine hydrochloride salt (171.8 mg) as a light brown solid. ¹HNMR (400 MHz, DMSO-d6) δ (ppm): 8.40 (s, 1H), 7.51-7.39 (m, 2H), 7.30-7.26 (m, 3H), 7.22 (ddd, J=8.8, 5.6, 2.6 Hz, 2H), 5.77 (p, J=7.9 Hz, 1H), 4.17 (q, J=6.9 Hz, 2H), 3.50 (d, J=7.9 Hz, 4H), 1.32 (t, J=7.0 Hz, 3H). LCMS: 456 (M+1).

### Example 77: Preparation of Compound No. 77

### Synthesis of 3-(3-chloro-4-ethoxy-5-methoxyphenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (3-chloro-4-ethoxy-5-methoxy-phenyl)boronic acid (275 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(3-chloro-4-ethoxy-5-methoxy-phenyl)-1-indan-2-yl-pyrazolo[3,4-d]pyrimidin-4-amine (160 mg) as an off-white solid, dissolved in 10 mL ethanolic HCl and concentrated under reduced pressure to obtain 3-(3-chloro-4-ethoxy-5-methoxy-phenyl)-1-indan-2-yl-pyrazolo[3,4-d]pyrimidin-4-amine hydrochloride salt (162 mg) as a light brown solid. ¹HNMR (400 MHz, DMSO-d6) δ (ppm): 8.36 (s, 1H), 7.30-7.24 (m, 2H), 7.24-7.18 (m, 4H), 5.75 (p, J=8.0 Hz, 1H), 4.07 (q, J=7.0 Hz, 2H), 3.86 (s, 3H), 3.49 (d, J=8.0 Hz, 4H), 1.32 (t, J=7.0 Hz, 3H). LCMS: 436 (M+1).

### Example 78: Preparation of Compound No. 78

### Synthesis of 1-(2,3-dihydro-1H-inden-2-yl)-3-(1H-indol-4-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 4-(4,4,5,5-tetramethyl-1,3,2-dioxaborolan-2-yl)-1H-indole (290.03 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 108 mg of 1-(2,3-dihydro-1H-inden-2-yl)-3-(1H-indol-5-yl)-1H-pyrazolo[3,4-d]pyrimidin-4-amine as a light brown solid. To this was added ethanolic HCl (10 mL) and stirred for 30 min at RT. The reaction mixture was then concentrated under reduced pressure and lyophilized to afford 1-(2,3-dihydro-1H-inden-2-yl)-3-(1H-indol-5-yl)-1H-pyrazolo[3,4-d]pyrimidin-4-amine (109.5 mg) as the HCl salt (light brown solid). ¹HNMR (400 MHz, DMSO-d6) δ (ppm): 11.38 (s, 1H), 8.42 (s, 1H), 7.54 (d, J=7.9 Hz, 2H), 7.42 (t, J=2.8 Hz, 1H), 7.28 (m, 2H), 7.27-7.13 (m, 3H), 6.41 (d, J=2.5 Hz, 1H), 5.81 (p, J=7.7 Hz, 1H), 3.59-3.47 (m, 4H).

### Example 79: Preparation of Compound No. 79

### Synthesis of 3-[3-chloro-5-ethoxy-4-(propan-2-yloxy)phenyl]-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3-chloro-5-ethoxy-4-isopropoxyphenylboronic acid (308.4 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 126 mg of 3-(3-chloro-5-ethoxy-4-isopropoxyphenyl)-1-(2,3-dihydro-1H-inden-2-yl)-1H-pyrazolo[3,4-d]pyrimidin-4-amine as a light brown solid. To this was added ethanolic HCl (10 mL) and stirred for 30 min at RT. The reaction mixture was then concentrated under reduced pressure and lyophilized to afford 3-(3-chloro-5-ethoxy-4-isopropoxyphenyl)-1-(2,3-dihydro-1H-inden-2-yl)-1H-pyrazolo[3,4-d]pyrimidin-4-amine (128 mg) as the HCl salt (light brown solid). ¹HNMR (400 MHz, DMSO-d6) δ (ppm): 8.48 (s, 1H), 7.28 (m, 3H), 7.21 (m, 4H), 5.77 (p, J=7.8 Hz, 1H), 4.51 (m, 1H), 4.09 (q, 2H), 3.50 (m, 4H), 1.38 (t, 3H), 1.36-1.24 (m, 6H).

### Example 80: Preparation of Compound No. 80

### Synthesis of 3-(3-chloro-4,5-dimethoxyphenyl)-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3-chloro-4,5-dimethoxyphenylboronic acid (258.1 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC affording 41 mg of 3-(3-chloro-4,5-dimethoxyphenyl)-1-(2,3-dihydro-1H-inden-2-yl)-1H-pyrazolo[3,4-d]pyrimidin-4-amine as a light brown solid. To this was added ethanolic HCl (10 mL) and stirred for 30 min at RT. The reaction mixture was then concentrated under reduced pressure and lyophilized to afford 3-(3-chloro-4,5-dimethoxyphenyl)-1-(2,3-dihydro-1H-inden-2-yl)-1H-pyrazolo[3,4-d]pyrimidin-4-amine (42 mg) as the HCl salt (light brown solid). ¹HNMR (400 MHz, DMSO-d6) δ (ppm): 8.34 (s, 1H), 7.30-7.21 (m, 6H), 5.81 (t, J=8.1 Hz, 1H), 3.93 (s, 3H), 3.89 (s, 3H), 3.67-3.59 (m, 2H), 3.59-3.43 (m, 2H).

### Example 81: Preparation of Compound No. 81

### Synthesis of 5-[4-amino-1-(2,3-dihydro-1H-inden-2-yl)-1H-pyrazolo[3,4-d]pyrimidin-3-yl]-2,3-difluorophenol

To a solution of 1-indan-2-yl-3-iodo-pyrazolo[3,4-d]pyrimidin-4-amine (300 mg, 0.795 mmol) and (3,4-difluoro-5-hydroxy-phenyl)boronic acid (207 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2,3-difluoro-phenol (95 mg) as an off-white solid, which was dissolved in 5 mL ethanolic HCl and concentrated under reduced pressure to Synthesis of 5-(4-amino-1-indan-2-yl-pyrazolo[3,4-d]pyrimidin-3-yl)-2,3-difluoro-phenol hydrochloride salt (57 mg) as a light brown solid. ¹HNMR (400 MHz, DMSO-d6) δ (ppm): 10.71 (s, 1H), 8.36 (s, 1H), 7.27 (dd, J=5.4, 3.3 Hz, 2H), 7.20 (dd, J=5.5, 3.3 Hz, 2H), 7.02 (dq, J=9.6, 3.4 Hz, 2H), 5.74 (p, J=7.7 Hz, 1H), 3.48 (d, J=7.9 Hz, 4H). LCMS: 380 (M+1).

### Example 82: Preparation of Compound No. 82

### Synthesis of 1-(2,3-dihydro-1H-inden-2-yl)-3-(2-methyl-1H-1,3-benzodiazol-6-yl)-1H-pyrazolo[3,4-d]pyrimidin-4-amine

To a solution of 1-(2, 3-dihydro-1H-inden-2-yl)-3-iodo-1H-pyrazolo[3,4-d]pyrimidin-4-amine (300 mg, 0.795 mmol) in DMF (5 mL) was added 2-methyl-5-(4,4,5,5-tetramethyl-1,3-dioxolan-2-yl)-1H-benzo[d]imidazole (310.05 mg, 1.19 mmol) at RT. Then Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layer was diluted with water (2×50 mL), brine (50 mL) then dried over sodium sulfate and concentrated under reduced pressured. The crude compound was purified by preparative HPLC affording 1-(2, 3-dihydro-1H-inden-2-yl)-3-(2-methyl-1H-benzo[d]imidazol-5-yl)-1H-pyrazolo[3,4-d]pyrimidin-4-amine as the free base. To this was added ethanolic HCl (10 mL) and stirred for 30 min at RT. The reaction mixture was then concentrated under reduced pressure and lyophilized to afford 1-(2, 3-dihydro-1H-inden-2-yl)-3-(2-methyl-1H-benzo[d]imidazol-5-yl)-1H-pyrazolo[3,4-d] pyrimidin-4-amine (54 mg) as the HCl salt. ¹HNMR (400 MHz, Methanol-d4) δ (ppm): 8.48 (s, 1H), 8.04 (s, 1H), 7.94-7.84 (m, 2H), 7.32-7.25 (m, 2H), 7.24-7.16 (m, 2H), 5.91 (q, J=7.7 Hz, 1H), 3.58 (d, J=16.0, 8.6 Hz, 4H), 2.92 (s, 3H).

### Example 83: Preparation of Compound No. 83

### Synthesis of 1-(2,3-dihydro-1H-inden-2-yl)-3-(2,3,4-trimethoxyphenyl)-1H-pyrazolo[3,4-d]pyrimidin-4-amine

To a solution of 1-(2, 3-dihydro-1H-inden-2-yl)-3-iodo-1H-pyrazolo[3,4-d] pyrimidin-4-amine (300 mg, 0.795 mmol) in DMF (5 mL) was added 2,3,4-trimethoxyphenylboronic acid (253 mg, 1.19 mmol) at RT. Then Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layer was diluted with water (2×50 mL), brine (50 mL) then dried over anhydrous sodium sulfate and concentrated under reduced pressure. The crude compound was purified by preparative HPLC affording 1-(2, 3-dihydro-1H-inden-2-yl)-3-(2, 3, 4-trimethoxyphenyl)-1H-pyrazolo [3,4-d] pyrimidin-4-amine as the free base. To this was added ethanolic HCl (10 mL) and stirred for 30 min RT. The reaction mixture was then concentrated under reduced pressure and lyophilized to afford 1-(2, 3-dihydro-1H-inden-2-yl)-3-(2, 3, 4-trimethoxyphenyl)-1H-pyrazolo [3,4-d] pyrimidin-4-amine (130 mg) as the HCl salt. ¹HNMR (400 MHz, Methanol-d4) δ (ppm): 8.41 (s, 1H), 7.25 (dd, J=7.1, 3.5 Hz, 2H), 7.20 (m, 2H), 6.96 (d, J=8.7 Hz, 1H), 5.86 (m, 1H), 3.92 (d, J=1.0 Hz, 5H), 3.73 (s, 3H), 3.63-3.47 (m, 4H).

### Example 84: Preparation of Compound No. 84

### Synthesis of 1-(2,3-dihydro-1H-inden-2-yl)-3-(8-fluoro-3,4-dihydro-2H-1,4-benzoxazin-6-yl)-1H-pyrazolo[3,4-d]pyrimidin-4-amine

To a solution of 1-(2, 3-dihydro-1H-inden-2-yl)-3-iodo-1H-pyrazolo [3,4-d] pyrimidin-4-amine (300 mg, 0.795 mmol) in DMF (5 mL) was added 8-fluoro-3, 4-dihydro-2H-benzo[b][1,4]oxazin-6-ylboronic acid (234 mg, 1.19 mmol) at RT. Then Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. reaction was monitored by TLC and by LCMS. After completion of reaction, the reaction mixture was diluted with water (40 mL) and extracted with EtOAc (2×100 mL). The combined organic layer was diluted with water (2×50 mL), brine (50 mL) then dried over sodium sulfate and concentrated under reduced pressured. The crude compound was purified by preparative HPLC affording 1-(2,3-dihydro-1H-inden-2-yl)-3-(8-fluoro-3,4-dihydro-2H-benzo[b][1,4]oxazin-6-yl)-1H-pyrazolo[3,4-d]pyrimidin-4-amine as the free base. To this was added ethanolic HCl (10 mL) and stirred for 30 min RT. The reaction mixture was then concentrated under reduced pressure and lyophilized to afford 1-(2, 3-dihydro-1H-inden-2-yl)-3-(8-fluoro-3, 4-dihydro-2H-benzo[b][1,4]oxazin-6-yl)-1H-pyrazolo[3,4-d]pyrimidin-4-amine (130 mg) as the HCl salt. ¹HNMR (400 MHz, Methanol-d4) δ (ppm): 8.42 (s, 1H), 7.33-7.22 (m, 2H), 7.25-7.15 (m, 2H), 6.85-6.92 (m, 2H), 5.84 (q, J=7.9 Hz, 1H), 4.31 (t, J=4.3 Hz, 2H), 3.66-3.41 (m, 4H), 3.24-3.34 (m, 2H).

### Example 85: Preparation of Compound No. 85

### Synthesis of 4-[4-amino-1-(2,3-dihydro-1H-inden-2-yl)-1H-pyrazolo[3,4-d]pyrimidin-3-yl]-2-methoxyphenol

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 4-hydroxy-3-methoxyphenylboronic acid (200.4 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and LCMS. After completion of reaction, the reaction mixture was diluted with water (40 mL) and extracted with EtOAc (2×100 mL). The combined organic layers were washed with water (2×50 mL), brine (50 mL), dried over anhydrous sodium sulfate and concentrated under reduced pressure. The crude compound was purified by preparative HPLC to obtain 4-(4-amino-1-(2,3-dihydro-1H-inden-2-yl)-1H-pyrazolo[3,4-d]pyrimidin-3-yl)-2-methoxyphenol (44 mg) as an off-white solid. To this was added ethanolic HCl (10 mL) and stirred for 30 min at RT. The reaction mixture was then concentrated under reduced pressure and lyophilized to afford 4-(4-amino-1-(2,3-dihydro-1H-inden-2-yl)-1H-pyrazolo[3,4-d]pyrimidin-3-yl)-2-methoxyphenol (45 mg) as the HCl salt (off-white solid). ¹HNMR (400 MHz, Methanol-d4) δ (ppm): 8.41 (s, 1H), 7.30-7.16 (m, 5H), 7.19-7.08 (m, 1H), 6.98 (d, J=8.1 Hz, 1H), 5.86 (t, J=8.1 Hz, 1H), 3.91 (s, 3H), 3.63 (dd, J=15.9, 7.8 Hz, 2H), 3.57-3.46 (m, 2H).

### Example 86: Preparation of Compound No. 86

### Synthesis of 1-(2,3-dihydro-1H-inden-2-yl)-3-(3,4,5-trifluorophenyl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3,4,5-trifluorophenylboronic acid (209.3 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated under reduced pressure. The crude compound was purified by preparative HPLC to obtain 1-(2,3-dihydro-1H-inden-2-yl)-3-(3,4,5-trifluorophenyl)-1H-pyrazolo[3,4-d]pyrimidin-4-amine (134 mg) as an off-white solid. To this was added ethanolic HCl (10 mL) and stirred for 30 min at RT. The reaction mixture was then concentrated under reduced pressure and lyophilized to afford 1-(2,3-dihydro-1H-inden-2-yl)-3-(3,4,5-trifluorophenyl)-1H-pyrazolo[3,4-d]pyrimidin-4-amine (137 mg) as the HCl salt (off-white solid). ¹HNMR (400 MHz, Methanol-d4) δ (ppm): 8.43 (s, 1H), 7.54-7.38 (m, 2H), 7.33-7.07 (m, 4H), 5.87 (p, J=7.8 Hz, 1H), 3.57 (qd, J=16.0, 7.8 Hz, 4H).

### Example 87: Preparation of Compound No. 87

### Synthesis of 3-(3-amino-4-fluorophenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (3-amino-4-fluoro-phenyl)boronic acid (186 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to 3-(3-amino-4-fluoro-phenyl)-1-indan-2-yl-pyrazolo[3,4-d]pyrimidin-4-amine (148 mg) as an off-white solid, which was dissolved in ethanolic HCl (10 mL) and concentrated under reduced pressure 3-(3-amino-4-fluoro-phenyl)-1-indan-2-yl-pyrazolo[3,4-d]pyrimidin-4-amine hydrochloride salt (154 mg) as an off-white solid. ¹HNMR (400 MHz, Methanol-d4) δ (ppm): 8.44 (s, 1H), 7.34 (dd, J=8.1, 2.1 Hz, 1H), 7.32-7.15 (m, 5H), 5.88 (p, J=7.9 Hz, 1H), 3.57 (qd, J=16.0, 7.9 Hz, 4H). LCMS: 461 (M+1).

### Example 88: Preparation of Compound No. 88

### Synthesis of 1-(2,3-dihydro-1H-inden-2-yl)-3-(3-fluorophenyl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (3-fluorophenyl) boronic acid (166.9 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(3-fluorophenyl)-1-indan-2-yl-pyrazolo[3,4-d]pyrimidin-4-amine (76 mg) as an off-white solid, which was dissolved in ethanolic HCl (10 mL) and concentrated under reduced pressure 3-(3-fluorophenyl)-1-indan-2-yl-pyrazolo[3,4-d]pyrimidin-4-amine hydrochloride salt (77 mg) as an off-white solid. ¹HNMR (400 MHz, Methanol-d4) δ (ppm): 8.44 (s, 1H), 7.59 (td, J=8.0, 5.7 Hz, 1H), 7.50 (dt, J=7.7, 1.3 Hz, 1H), 7.43 (dt, J=9.5, 2.1 Hz, 1H), 7.28 (ddd, J=12.8, 6.7, 2.6 Hz, 3H), 7.24-7.13 (m, 2H), 5.87 (p, J=7.9 Hz, 1H), 3.62 (dd, J=15.9, 7.4 Hz, 2H), 3.53 (dd, J=15.9, 8.3 Hz, 2H). LCMS: −346 (M+1).

### Example 89: Preparation of Compound No. 89

### Synthesis of 3-(3,4-difluorophenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and 3,4-difluorophenyl boronic acid (188.29 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(3,4-difluorophenyl)-1-indan-2-yl-pyrazolo[3,4-d]pyrimidin-4-amine (97 mg) as an off-white solid, which was dissolved in ethanolic HCl (10 mL) and concentrated under reduced pressure to 3-(3,4-difluorophenyl)-1-indan-2-yl-pyrazolo[3,4-d]pyrimidin-4-amine hydrochloride salt (105 mg) as an off-white solid. ¹HNMR (400 MHz, Methanol-d4) δ (ppm): 8.38 (s, 1H), 7.64-7.53 (m, 1H), 7.54-7.33 (m, 2H), 7.32-7.22 (m, 2H), 7.23-7.09 (m, 2H), 5.83 (p, J=8.0 Hz, 1H), 3.60 (dd, J=15.9, 7.6 Hz, 2H), 3.50 (dd, J=15.9, 8.3 Hz, 2H). LCMS: 364 (M+1).

### Example 90: Preparation of Compound No. 90

### Synthesis of 3-(3-butoxy-4-methoxyphenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (4-butoxy-3-methoxy-phenyl)boronic acid (267.5 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(3-butoxy-4-methoxy-phenyl)-1-indan-2-yl-pyrazolo[3,4-d]pyrimidin-4-amine (125 mg) as an off-white solid, which was dissolved in ethanolic HCl (10 mL) and concentrated under reduced pressure to 3-(3-butoxy-4-methoxy-phenyl)-1-indan-2-yl-pyrazolo[3,4-d]pyrimidin-4-amine hydrochloride salt (129.2 mg) as a light brown solid. ¹HNMR (400 MHz, Methanol-d4) δ (ppm): 8.42 (s, 1H), 7.29-7.18 (m, 6H), 7.13 (d, J=8.3 Hz, 1H), 5.86 (p, J=8.0 Hz, 1H), 4.04 (t, J=6.4 Hz, 2H), 3.91 (s, 3H), 3.62 (dd, J=15.9, 7.6 Hz, 2H), 3.51 (dd, J=16.0, 8.4 Hz, 2H), 1.79 (p, J=6.7 Hz, 2H), 1.52 (h, J=7.4 Hz, 2H), 0.99 (t, J=7.4 Hz, 3H). LCMS: 430 (M+1).

### Example 91: Preparation of Compound No. 91

### Synthesis of 3-(3,4-difluoro-5-methoxyphenyl)-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3,4-difluoro-5-methoxyphenylboronic acid (223.6 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to afford 3-(3,4-difluoro-5-methoxyphenyl)-1-(2,3-dihydro-1H-inden-2-yl)-1H-pyrazolo[3,4-d]pyrimidin-4-amine (46 mg) as an off-white solid. To this was added ethanolic HCl (10 mL) and stirred for 30 min at RT. The reaction mixture was then concentrated under reduced pressure and lyophilized to afford 3-(3,4-difluoro-5-methoxyphenyl)-1-(2,3-dihydro-1H-inden-2-yl)-1H-pyrazolo[3,4-d]pyrimidin-4-amine (48 mg) as the HCl salt (off-white solid). ¹HNMR (400 MHz, Methanol-d4) δ (ppm): 8.43 (s, 1H), 7.33-7.09 (m, 6H), 5.87 (p, J=7.9 Hz, 1H), 3.96 (s, 3H), 3.63-3.44 (m, 4H).

### Example 92: Preparation of Compound No. 92

### Synthesis of 1-(2,3-dihydro-1H-inden-2-yl)-3-[4-fluoro-3-methoxy-5-(trifluoromethyl) phenyl]-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 4-fluoro-3-methoxy-5-(trifluoromethyl)phenylboronic acid (283.8 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over anhydrous sodium sulfate and concentrated. The crude compound was purified by preparative HPLC to afford 1-(2,3-dihydro-1H-inden-2-yl)-3-(4-fluoro-3-methoxy-5-(trifluoromethyl)phenyl)-1H-pyrazolo[3,4-d]pyrimidin-4-amine (86 mg) as an off-white solid. To this was added ethanolic HCl (10 mL) and stirred for 30 min at RT. The reaction mixture was then concentrated under reduced pressure and lyophilized to afford 1-(2,3-dihydro-1H-inden-2-yl)-3-(4-fluoro-3-methoxy-5-(trifluoromethyl)phenyl)-1H-pyrazolo[3,4-d]pyrimidin-4-amine (87.5 mg) as the HCl salt (off-white solid). ¹HNMR (400 MHz, Methanol-d4) δ (ppm): 8.33 (s, 1H), 7.65 (d, J=7.4 Hz, 1H), 7.48 (d, J=5.5 Hz, 1H), 7.25 (d, J=4.4 Hz, 2H), 7.24-7.16 (m, 2H), 5.82 (p, J=8.0 Hz, 1H), 3.99 (d, J=1.1 Hz, 3H), 3.62 (dd, J=15.8, 7.8 Hz, 2H), 3.49 (dd, J=15.9, 8.4 Hz, 2H).

### Example 93: Preparation of Compound No. 93

### Synthesis of 5-[4-amino-7-(2,3-dihydro-1H-inden-2-yl)-7H-pyrrolo[2,3-d]pyrimidin-5-yl]-2-chlorophenol

Steps 1-3 are same as in Example 62.

### Step 4: 5-(4-amino-7-(2,3-dihydro-1H-inden-2-yl)-7H-pyrrolo[2,3-d]pyrimidin-5-yl)-2-chlorophenol

To a solution of 5-bromo-7-(2,3-dihydro-1H-inden-2-yl)-7H-pyrrolo[2,3-d]pyrimidin-4-amine (180 mg, 0.546 mmol) in DMF (4 mL) was added 4-chloro-3-hydroxyphenylboronic acid (142 mg, 0.819 mmol) at RT. Then, Na₂CO₃ (174 mg, 1.64 mmol) dissolved in water (4 mL) was added to the reaction mixture followed by addition of Pd(PPh₃)₄ (38 mg, 0.032 mmol) at RT and the resultant reaction mixture was heated at 100° C. for 1 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 5-(4-amino-7-(2,3-dihydro-1H-inden-2-yl)-7H-pyrrolo[2,3-d]pyrimidin-5-yl)-2-chlorophenol (35 mg) as an off-white solid. To this was added ethanolic HCl (10 mL) and stirred for 30 min at RT. The reaction mixture was then concentrated under reduced pressure and lyophilized to obtain 5-(4-amino-7-(2,3-dihydro-1H-inden-2-yl)-7H-pyrrolo[2,3-d]pyrimidin-5-yl)-2-chlorophenol (36 mg) as the HCl salt (off-white solid). ¹HNMR (400 MHz, Methanol-d4) δ (ppm): 8.35 (s, 1H), 7.40 (d, J=8.1 Hz, 1H), 7.36-7.26 (m, 3H), 7.24 (dd, J=5.6, 3.2 Hz, 2H), 6.96 (d, J=2.1 Hz, 1H), 6.88 (dd, J=8.1, 2.0 Hz, 1H), 5.84-5.73 (m, 1H), 3.59 (dd, J=16.2, 7.7 Hz, 2H), 3.37 (dd, J=16.2, 5.7 Hz, 2H).

### Example 94: Preparation of Compound No. 94

### Synthesis of 4-[4-amino-1-(2,3-dihydro-1H-inden-2-yl)-1H-pyrazolo[3,4-d]pyrimidin-3-yl]-2-hydroxybenzonitrile

To a solution of 1-indan-2-yl-3-iodo-pyrazolo[3,4-d]pyrimidin-4-amine (300 mg, 0.795 mmol) and 2-hydroxy-4-(4,4,5,5-tetramethyl-1,3,2-dioxaborolan-2-yl)benzonitrile (291.66 mg, 1.193 mmol) in DMF (5 mL) was added a solution of sodium carbonate (253 mg, 2.387 mmol) in water (5 mL) followed by the addition of tetrakis(triphenylphosphine) palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 4-(4-amino-1-indan-2-yl-pyrazolo[3,4-d]pyrimidin-3-yl)-2-hydroxy-benzonitrile (90 mg) as a light brown solid was dissolved ethanolic HCl (10 mL) and concentrated under reduced pressure 4-(4-amino-1-indan-2-yl-pyrazolo[3,4-d]pyrimidin-3-yl)-2-hydroxy-benzonitrile hydrochloride salt (94 mg) as a light brown solid. ¹HNMR (400 MHz, Methanol-d4) δ (ppm): 8.44 (s, 1H), 7.70 (d, J=7.9 Hz, 1H), 7.66-7.52 (m, 2H), 7.25 (dq, J=3.2, 1.9 Hz, 2H), 7.21 (td, J=5.6, 4.9, 2.3 Hz, 2H), 5.88 (p, J=7.8 Hz, 1H), 3.56 (dp, J=16.0, 8.5 Hz, 4H). LCMS: 367 (M+1).

### Example 95: Preparation of Compound No. 95

### Synthesis of 3-(2,4-dichloro-3-methoxyphenyl)-1-(2,3-dihydro-1H-inden-2-yl)-1H-pyrazolo[3,4-d]pyrimidin-4-amine

To a solution of 1-indan-2-yl-3-iodo-pyrazolo[3,4-d]pyrimidin-4-amine (300 mg, 0.795 mmol) and (2,4-dichloro-3-methoxy-phenyl)boronic acid (220.8 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(2,4-dichloro-3-methoxy-phenyl)-1-indan-2-yl-pyrazolo[3,4-d]pyrimidin-4-amine (21 mg) as an off-white solid, which was dissolved ethanolic HCl (10 mL) and concentrated under reduced pressure 3-(2,4-dichloro-3-methoxy-phenyl)-1-indan-2-yl-pyrazolo[3,4-d]pyrimidin-4-amine hydrochloride salt (23 mg) as an off-white solid. ¹HNMR (400 MHz, Methanol-d4) δ (ppm): 8.37 (s, 1H), 7.54 (d, J=8.3 Hz, 1H), 7.34-7.22 (m, 2H), 7.26-7.13 (m, 3H), 5.85 (p, J=8.0 Hz, 1H), 3.95 (s, 3H), 3.56 (qd, J=16.0, 8.0 Hz, 4H). LCMS: 427 (M+1).

### Example 96: Preparation of Compound No. 96

### Synthesis of 5-[4-amino-1-(2,3-dihydro-1H-inden-2-yl)-1H-pyrazolo[3,4-d]pyrimidin-3-yl]pyridin-2-amine

To a solution of 1-indan-2-yl-3-iodo-pyrazolo[3,4-d]pyrimidin-4-amine (300 mg, 0.795 mmol) and 5-(4,4,5,5-tetramethyl-1,3,2-dioxaborolan-2-yl)pyridin-2-amine (262.5 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(6-amino-3-pyridyl)-1-indan-2-yl-pyrazolo[3,4-d]pyrimidin-4-amine (105 mg) as an off-white solid, which was dissolved ethanolic HCl (10 mL) and concentrated under reduced pressure 3-(6-amino-3-pyridyl)-1-indan-2-yl-pyrazolo[3,4-d]pyrimidin-4-amine trihydrochloride salt (111.5 mg) as an off-white solid. ¹HNMR (400 MHz, Methanol-d4) δ (ppm): 8.47 (d, J=1.0 Hz, 1H), 8.16 (m, 2H), 7.30-7.08 (m, 5H), 5.89 (p, J=7.7 Hz, 1H), 3.56 (d, J=7.7 Hz, 4H). LCMS: 344 (M+1).

### Example 97: Preparation of Compound No. 97

### Synthesis of 5-[4-amino-1-(2,3-dihydro-1H-inden-2-yl)-1H-pyrazolo[3,4-d]pyrimidin-3-yl]-2-fluorobenzonitrile

To a solution of 1-indan-2-yl-3-iodo-pyrazolo[3,4-d]pyrimidin-4-amine (300 mg, 0.795 mmol) and 3-cyano-4-fluorophenyl boronic acid (294.82 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2-fluoro-benzonitrile (23 mg) as an off-white solid, which was dissolved ethanolic HCl (10 mL) and concentrated under reduced pressure 5-(4-amino-1-indan-2-yl-pyrazolo[3,4-d]pyrimidin-3-yl)-2-fluoro-benzonitrile hydrochloride salt (25 mg) as an off-white solid. ¹HNMR (400 MHz, Methanol-d4) δ (ppm): 8.44 (s, 1H), 8.07 (dd, J=6.0, 2.3 Hz, 1H), 8.01 (ddd, J=7.7, 5.1, 2.3 Hz, 1H), 7.54 (t, J=8.9 Hz, 1H), 7.32-7.24 (m, 2H), 7.23-7.14 (m, 2H), 5.88 (p, J=7.7 Hz, 2H), 3.70-3.58 (m, 2H), 3.54 (m, 2H). LCMS: 371 (M+1).

### Example 98: Preparation of Compound No. 98

### Synthesis of 1-(2,3-dihydro-1H-inden-2-yl)-3-(1-methyl-1H-indazol-4-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 1-methyl-1H-indazol-4-ylboronic acid (209.4 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 1-(2, 3-dihydro-1H-inden-2-yl)-3-(1-methyl-1H-indazol-4-yl)-1H-pyrazolo [3,4-d] pyrimidin-4-amine (24 mg) as an off-white solid. To this was added ethanolic HCl (10 mL) and stirred for 30 min at RT. The reaction mixture was then concentrated under reduced pressure and lyophilized to obtain 1-(2, 3-dihydro-1H-inden-2-yl)-3-(1-methyl-1H-indazol-4-yl)-1H-pyrazolo [3,4-d] pyrimidin-4-amine (25 mg) as the HCl salt (off-white solid). ¹HNMR (400 MHz, Methanol-d4) δ (ppm): 8.46 (s, 1H), 7.86 (s, 1H), 7.73 (d, J=8.5 Hz, 1H), 7.65-7.54 (m, 1H), 7.44 (d, J=7.1 Hz, 1H), 7.34-7.26 (m, 1H), 7.26-7.17 (m, 1H), 5.92 (p, J=7.1 Hz, 1H), 4.12 (s, 3H), 3.61 (d, J=7.2 Hz, 4H).

### Example 99: Preparation of Compound No. 99

### Synthesis of 1-(2,3-dihydro-1H-inden-2-yl)-3-(3,4-dihydro-2H-1,4-benzoxazin-6-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 3,4-dihydro-2H-benzo[b][1,4]oxazin-6-ylboronic acid (213.01 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 1-(2,3-dihydro-1H-inden-2-yl)-3-(3,4-dihydro-2H-benzo[b][1,4]oxazin-6-yl)-1H-pyrazolo[3,4-d]pyrimidin-4-amine (57 mg) as an off-white solid. To this was added ethanolic HCl (10 mL) and stirred for 30 min at RT. The reaction mixture was then concentrated under reduced pressure and lyophilized to obtain 1-(2,3-dihydro-1H-inden-2-yl)-3-(3,4-dihydro-2H-benzo[b][1,4] oxazin-6-yl)-1H-pyrazolo[3,4-d]pyrimidin-4-amine (58.5 mg) as the HCl salt (off-white solid). ¹HNMR (400 MHz, Methanol-d4) δ (ppm): 8.42 (s, 1H), 7.27-7.24 (m, 2H), 7.24-7.17 (m, 2H), 7.10 (s, 1H), 7.06 (d, J=8.6 Hz, 1H), 6.97 (d, J=8.2 Hz, 1H), 5.85 (q, J=7.8 Hz, 1H), 4.32 (t, J=4.5 Hz, 2H), 3.60 (dd, J=15.9, 7.5 Hz, 2H), 3.52 (dd, J=16.5, 7.2 Hz, 2H).

### Example 100: Preparation of Compound No. 100

### Synthesis of 3-[4-amino-1-(2,3-dihydro-1H-inden-2-yl)-1H-pyrazolo[3,4-d]pyrimidin-3-yl]-6-chloro-2-fluorophenol

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 4-chloro-2-fluoro-3-hydroxyphenylboronic acid (227.1 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 3-(4-amino-1-(2,3-dihydro-1H-inden-2-yl)-1H-pyrazolo[3,4-d]pyrimidin-3-yl)-6-chloro-2-fluorophenol (61 mg) as an off-white solid. To this was added ethanolic HCl (10 mL) and stirred for 30 min at RT. The reaction mixture was then concentrated under reduced pressure and lyophilized to obtain 3-(4-amino-1-(2,3-dihydro-1H-inden-2-yl)-1H-pyrazolo[3,4-d]pyrimidin-3-yl)-6-chloro-2-fluorophenol (62 mg) as the HCl salt (off-white solid). ¹HNMR (400 MHz, Methanol-d4) δ (ppm): 8.42 (s, 1H), 7.34-7.16 (m, 5H), 7.02 (dd, J=8.5, 7.0 Hz, 1H), 5.88 (p, J=7.9 Hz, 1H), 3.57 (qd, J=16.0, 7.9 Hz, 4H).

### Example 101: Preparation of Compound No. 101

### Synthesis of 5-[4-amino-7-(2,3-dihydro-1H-inden-2-yl)-7H-pyrrolo[2,3-d]pyrimidin-5-yl]-2,3-difluorophenol

To a stirred solution of 5-bromo-7-indan-2-yl-pyrrolo[2,3-d]pyrimidin-4-amine (220 mg, 0.67 mmol) and (3,4-difluoro-5-hydroxy-phenyl)boronic acid (196 mg, 1.139 mmol) in DMF (5 mL) was added a solution of sodium carbonate (213 mg, 2.01 mmol) in water (5 mL) and tetrakis(triphenylphosphine)palladium(0) (54.2 mg, 0.0469 mmol) at RT. The reaction mixture was allowed to heat at 80° C. for 1.5 h. After completion of reaction, the reaction mixture was allowed to cool, quenched with water (50 mL) followed by extraction with EtOAc (3×40 mL). The combined organic layers were washed with brine (100 mL), dried over anhydrous sodium sulfate, concentrated under reduced pressure to give a crude which was purified over reverse phase HPLC to give 5-(4-amino-7-indan-2-yl-pyrrolo[2,3-d]pyrimidin-5-yl)-2,3-difluoro-phenol (14 mg). To this was added ethanolic HCl (2.5 mL) to give 5-(4-amino-7-indan-2-yl-pyrrolo[2,3-d]pyrimidin-5-yl)-2,3-difluoro-phenol HCl Salt (14.2 mg). ¹HNMR (400 MHz, Methanol-d4) δ (ppm): 8.35 (s, 1H), 7.41 (s, 1H), 7.33-7.29 (m, 2H), 7.24 (dd, J=5.6, 3.2 Hz, 2H), 6.96 (d, J=2.1 Hz, 1H), 6.88 (dd, J=8.1, 2.1 Hz, 1H), 5.78 (t, J=6.0 Hz, 1H), 3.59 (dd, J=16.2, 7.7 Hz, 2H), 3.44-3.35 (m, 2H). LCMS (M+1): 379.

### Example 102: Preparation of Compound No. 102

### Synthesis of 5-[4-amino-1-(2,3-dihydro-1H-inden-2-yl)-1H-pyrazolo[3,4-d]pyrimidin-3-yl]-2-fluoro-3-(trifluoromethyl)phenol

To a suspension of 1-(2,3-dihydro-1H-inden-2-yl)-3-(4-fluoro-3-methoxy-5-(trifluoromethyl)phenyl)-1H-pyrazolo[3,4-d]pyrimidin-4-amine (50 mg, 0.112 mmol) in DCM (4 mL) was added BBr₃ (1M solution in DCM; 0.90 mL, 0.902 mmol) at 0° C. dropwise and the resultant reaction mixture was stirred at RT for 24 h. The reaction was monitored by TLC and by LCMS. After completion of reaction, the reaction mixture was quenched with ice-cold water and then acidified using 2N HCl (pH=2). The aq. layer was then extracted with EtOAc (2×100 mL). The combined organic layers were washed with brine (30 mL), dried over Na₂SO₄ and concentrated. The crude compound was purified by preparative HPLC to obtain 5-(4-amino-1-(2,3-dihydro-1H-inden-2-yl)-1H-pyrazolo[3,4-d]pyrimidin-3-yl)-2-fluoro-3-(trifluoromethyl) phenol (9.7 mg) as an off-white solid. To this was added ethanolic HCl (4 mL) and stirred for 20 min at RT. The reaction mixture was then concentrated under reduced pressure and lyophilized to obtain 5-(4-amino-1-(2,3-dihydro-1H-inden-2-yl)-1H-pyrazolo[3,4-d]pyrimidin-3-yl)-2-fluoro-3-(trifluoromethyl) phenol (10 mg) as the HCl salt (off-white solid). ¹HNMR (400 MHz, Methanol-d4) δ (ppm): 8.40 (s, 1H), 7.47 (d, J=8.0 Hz, 1H), 7.37 (d, J=5.7 Hz, 1H), 7.32-7.25 (m, 2H), 7.25-7.16 (m, 2H), 5.85 (p, J=7.9 Hz, 1H), 3.62 (d, J=15.9 Hz, 2H), 3.58-3.50 (m, 2H).

### Example 103: Preparation of Compound No. 103

### Synthesis of 5-[4-amino-1-(2,3-dihydro-1H-inden-2-yl)-1H-pyrazolo[3,4-d]pyrimidin-3-yl]-2,4-dichlorophenol

To a solution of 1-indan-2-yl-3-iodo-pyrazolo[3,4-d]pyrimidin-4-amine (300 mg, 0.795 mmol) and (2,4-dichloro-5-hydroxy-phenyl)boronic acid (247 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2,4-dichloro-phenol (32 mg) as an off-white solid, which was dissolved in 10 mL ethanolic HCl and concentrated under reduced pressure to give 5-(4-amino-1-indan-2-yl-pyrazolo[3,4-d]pyrimidin-3-yl)-2,4-dichloro-phenol hydrochloride salt (36.4 mg) as a light off-white solid. ¹HNMR (400 MHz, DMSO-d6) δ (ppm): 10.65 (s, 1H), 8.24 (s, 1H), 7.59 (s, 1H), 7.26 (dd, J=5.5, 3.3 Hz, 1H), 7.23-7.09 (m, 2H), 7.01 (d, J=8.6 Hz, 2H), 5.72 (p, J=7.7 Hz, 1H), 3.47 (qd, J=16.2, 7.8 Hz, 4H). LCMS: 412 (M+1).

### Example 104: Preparation of Compound No. 104

### Synthesis of 5-[4-amino-1-(2,3-dihydro-1H-inden-2-yl)-1H-pyrazolo[3,4-d]pyrimidin-3-yl]-2-chloropyridin-3-amine

To a solution of 1-indan-2-yl-3-iodo-pyrazolo[3,4-d]pyrimidin-4-amine (300 mg, 0.795 mmol) and (2,4-dichloro-5-hydroxy-phenyl)boronic acid (303.8 mg, 1.193 mmol) in DMF (5 mL) was added a solution of sodium carbonate (253 mg, 2.387 mmol) in water (5 mL) followed by the addition of tetrakis(triphenylphosphine)palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 3-(5-amino-6-chloro-3-pyridyl)-1-indan-2-yl-pyrazolo[3,4-d]pyrimidin-4-amine (17 mg) as an off-white solid, which was dissolved in 10 mL ethanolic HCl and concentrated under reduced pressure 3-(5-amino-6-chloro-3-pyridyl)-1-indan-2-yl-pyrazolo[3,4-d]pyrimidin-4-amine as the HCl salt (18 mg) as a light off-white solid. ¹HNMR (400 MHz, Methanol-d4) δ (ppm): 8.43 (s, 1H), 7.87 (d, J=2.1 Hz, 1H), 7.42 (d, J=2.1 Hz, 1H), 7.32-7.14 (m, 4H), 5.87 (p, J=7.8 Hz, 1H), 3.57 (qd, J=15.9, 7.9 Hz, 4H). LCMS: 378 (M+1).

### Example 105: Preparation of Compound No. 105

### Synthesis of 5-[4-amino-1-(2,3-dihydro-1H-inden-2-yl)-1H-pyrazolo[3,4-d]pyrimidin-3-yl]-2,3-dimethoxybenzonitrile

To a solution of 1-indan-2-yl-3-iodo-pyrazolo[3,4-d]pyrimidin-4-amine (300 mg, 0.795 mmol) and 2,3-dimethoxy-5-(4,4,5,5-tetramethyl-1,3,2-dioxaborolan-2-yl)benzonitrile (356 mg, 1.193 mmol) in DMF (5 mL) was added a solution of sodium carbonate (253 mg, 2.387 mmol) in water (5 mL) followed by the addition of tetrakis(triphenylphosphine) palladium(0) (92 mg, 0.0795 mmol). The reaction mixture was heated in a reagent bottle at 100° C. for 2 h. The reaction was monitored by TLC. After completion of reaction, water (45 mL) was added to the reaction mixture and the product was extracted with EtOAc (2×100 mL). The combined organic layer was again washed with water (100 mL) and finally with brine solution (2×75 mL). The organic layer was separated, dried over anhydrous sodium sulfate and concentrated under reduced pressure to afford a crude product which was purified by reverse phase HPLC to give 5-(4-amino-1-indan-2-yl-pyrazolo[3,4-d]pyrimidin-3-yl)-2,3-dimethoxy-benzonitrile (17 mg) as an off-white solid, which was dissolved in 10 mL ethanolic HCl and concentrated under reduced pressure 5-(4-amino-1-indan-2-yl-pyrazolo[3,4-d]pyrimidin-3-yl)-2,3-dimethoxy-benzonitrile as the HCl salt (50 mg) as a light off-white solid. ¹HNMR (400 MHz, Methanol-d4) δ (ppm): 8.41 (s, 1H), 7.56 (d, J=1.9 Hz, 1H), 7.48 (d, J=1.9 Hz, 1H), 7.26 (d, J=4.4 Hz, 2H), 7.25-7.16 (m, 2H), 5.85 (q, J=7.9 Hz, 1H), 4.07 (s, 3H), 3.95 (s, 3H), 3.62 (dd, J=15.9, 7.5 Hz, 2H), 3.52 (dd, J=16.1, 8.5 Hz, 2H). LCMS: 413 (M+1).

### Example 106: Preparation of Compound Nos. 106, 106a, 106b, 106c, and 106d

### Synthesis of 2-[4-amino-3-(4-chloro-3-hydroxyphenyl)-1H-pyrazolo[3,4-d]pyrimidin-1-yl]-2,3-dihydro-1H-inden-1-ol

2-[4-Amino-3-(4-chloro-3-hydroxy-phenyl)pyrazolo[3,4-d]pyrimidin-1-yl]indan-1-one (130 mg, 0.332 mmol) and (5 mL) ethanol were added into 25 mL two necked round bottom flask. The reaction mixture temperature was controlled below 10° C., and (18.8 mg 0.498 mmol) sodium borohydride was added in batches. Then the temperature was raised to RT, and the reaction mixture was continuously stirred for 3 h. The reaction mixture was monitored by LCMS. The ethanol was concentrated under reduced pressure and (0.5 mL) 10% HCl solution was added and the mixture extracted with DCM (3×3 mL). The combined organic layers were washed with water (2×5 mL), dried over anhydrous sodium sulfate and concentrated under reduced pressure to obtain a crude product which was purified by reverse phase HPLC to afford (5 mg) 2-[4-amino-3-(4-chloro-3-hydroxy-phenyl)pyrazolo[3,4-d]pyrimidin-1-yl]indan-1-ol as a white solid. ¹HNMR (400 MHz, Chloroform-d3) δ (ppm): 8.39 (s, 1H), 7.51 (d, J=21.7 Hz, 2H), 7.34 (s, 3H), 7.19 (d, J=8.2 Hz, 2H), 5.6 (m, 1H), 5.4 (m, 1H), 3.89 (d, J=16.0 Hz, 2H), 3.49 (d, J=16.0 Hz, 2H). LCMS (M+1): 393.7. Separation by chiral HPLC affords Compound Nos. 106a, 106b, 106c and 106d.

### Example 107: Preparation of Compound No. 107

### Synthesis of 5-[4-amino-7-(2,3-dihydro-1H-inden-2-yl)-7H-pyrrolo[2,3-d]pyrimidin-5-yl]-2,3-dichlorophenol

Steps 1-3 are same as in Example 62

### Step-4: Synthesis of (3,4-dichloro-5-hydroxy-phenyl)boronic acid

To a solution of 2-(3,4-dichloro-5-methoxy-phenyl)-4,4,5,5-tetramethyl-1,3,2-dioxaborolane (500 mg, 1.65 mmol) in DCM (10 mL) solution of BBr₃ in DCM (1 M, 6.6 mL) was added at 0° C., the reaction was allowed to warm to RT and stirred for 16 h, monitored by TLC. The reaction mixture was quenched with ice-cold water (150 mL) and then acidified with 3N aq. HCl (pH=2). The reaction mixture was extracted with EtOAc (2×50 mL). The organic layer was washed with brine (50 mL), dried over anhydrous sodium sulfate and concentrated under reduced pressure to obtain a crude product which was purified by washing with pentane (2×10 mL) to obtain 250 mg of (3,4-dichloro-5-hydroxy-phenyl)boronic acid as an off-white solid.

### Step-5: Synthesis of 5-(4-amino-7-indan-2-yl-pyrrolo[2,3-d]pyrimidin-5-yl)-2,3-dichloro-phenol

Na₂CO₃ (251 mg, 2.368 mmol) dissolved in water (5 mL) was added to a solution of (3,4-dichloro-5-hydroxy-phenyl)boronic acid (245 mg, 1.184 mmol) in DMF (5 mL). The reaction mixture was purged with nitrogen for 15 min, followed by addition of 5-bromo-7-indan-2-yl-pyrrolo[2,3-d]pyrimidin-4-amine (260 mg, 0.789 mmol) and tetrakis(triphenylphosphine)palladium(0) (55 mg, 0.047 mmol) and again purged for 2 minutes. The reaction was heated at 80° C. for 3 h, monitored by TLC. The reaction was diluted with water (25 mL) and extracted with EtOAc (2×40 mL). The combined organic layers were washed with water (2×60 mL) and brine (20 mL), dried over anhydrous sodium sulfate and concentrated under reduced pressure to afford a crude product which was purified by reverse phase preparative HPLC and treated with ethanolic HCl to obtain 5-(4-amino-7-indan-2-yl-pyrrolo[2,3-d]pyrimidin-5-yl)-2,3-dichloro-phenol (88 mg) as a white solid. ¹HNMR (400 MHz, DMSO-d6) δ (ppm): 10.92 (s, 1H), 8.47 (s, 1H), 7.94 (s, 2H), 7.65 (s, 1H), 7.29 (m, 2H), 7.22 (m, 2H), 7.12 (d, J=1.9 Hz, 1H), 6.97 (d, J=2.0 Hz, 1H), 5.62 (p, J=7.5 Hz, 1H), 3.48 (dd, J=16.1, 7.9 Hz, 4H).

### Example 108: Preparation of Compound No. 108

### Synthesis of 5-[4-amino-1-(2,3-dihydro-1H-inden-2-yl)-1H-pyrazolo[3,4-d]pyrimidin-3-yl]-1H-1,3-benzodiazol-2-ol

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 5-(4,4,5,5-tetramethyl-1,3,2-dioxaborolan-2-yl)-1H-benzo[d]imidazol-2-ol (310 mg, 1.19 mmol) at RT. Then, Na₂CO₃ (167 mg, 1.58 mmol) dissolved in water (5 mL) was added to the reaction mixture followed by addition of Pd(PPh₃)₄ (91.2 mg, 0.079 mmol) at RT and the resultant reaction mixture was heated at 110° C. for 2 h. The reaction was monitored by TLC and by LCMS. After completion of reaction, the reaction mixture was diluted with water (100 mL) and extracted with EtOAc (3×70 mL). The combined organic layers were washed with water (200 mL), brine (200 mL), dried over sodium sulfate and concentrated. The crude compound was purified by preparative HPLC to obtain 5-(4-amino-1-(2,3-dihydro-1H-inden-2-yl)-1H-pyrazolo[3,4-d]pyrimidin-3-yl)-1H-benzo[d]imidazol-2-ol (39 mg) as an off-white solid. To this was added ethanolic HCl (10 mL) and stirred for 30 min at RT. The reaction mixture was then concentrated under reduced pressure and lyophilized to obtain 5-(4-amino-1-(2,3-dihydro-1H-inden-2-yl)-1H-pyrazolo[3,4-d]pyrimidin-3-yl)-1H-benzo[d]imidazol-2-ol (40 mg) as the HCl salt (light brown solid). ¹HNMR (400 MHz, Methanol-d4) δ (ppm): 8.42 (s, 1H), 7.35 (d, J=7.9 Hz, 2H), 7.30-7.25 (m, 2H), 7.25-7.15 (m, 3H), 5.86 (q, J=8.0 Hz, 1H), 3.63 (dd, J=15.9, 7.5 Hz, 2H), 3.53 (dd, J=15.9, 8.4 Hz, 2H).

### Example 109: Preparation of Compound No. 109

### Synthesis of 3-(3-amino-4,5-difluorophenyl)-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added 2,3-difluoro-5-(4,4,5,5-tetramethyl-1,3,2-dioxaborolan-2-yl)aniline (304 mg, 1.19 mmol) at RT. Then, Na₂CO₃ (167 mg, 1.58 mmol) dissolved in water (5 mL) was added to the reaction mixture followed by addition of Pd(PPh₃)₄ (91.2 mg, 0.079 mmol) at RT and the resultant reaction mixture was heated at 110° C. for 2 h. The reaction was monitored by TLC and by LCMS. After completion of reaction, the reaction mixture was diluted with water (100 mL) and extracted with EtOAc (3×70 mL). The combined organic layers were washed with brine (300 mL), dried over sodium sulfate and concentrated. The crude compound was purified by preparative HPLC to obtain 3-(3-amino-4,5-difluorophenyl)-1-(2,3-dihydro-1H-inden-2-yl)-1H-pyrazolo[3,4-d]pyrimidin-4-amine 1(125 mg) as an off-white solid. To this was added ethanolic HCl (10 mL) and stirred for 30 min at RT. The reaction mixture was then concentrated under reduced pressure and lyophilized to obtain 3-(3-amino-4,5-difluorophenyl)-1-(2,3-dihydro-1H-inden-2-yl)-1H-pyrazolo[3,4-d]pyrimidin-4-amine (127 mg) as the HCl salt (light brown solid). ¹HNMR (400 MHz, Methanol-d4) δ (ppm): 8.43 (s, 1H), 7.33-7.24 (m, 2H), 7.24-7.15 (m, 2H), 6.95-6.86 (m, 1H), 6.77 (ddd, J=9.0, 6.6, 2.1 Hz, 1H), 5.85 (q, J=7.9 Hz, 1H), 3.60 (dd, J=15.9, 7.5 Hz, H), 3.52 (dd, J=16.0, 8.4 Hz, 2H).

### Example 110: Preparation of Compound No. 110

### Synthesis of 5-[4-amino-7-(2,3-dihydro-1H-inden-2-yl)-7H-pyrrolo[2,3-d]pyrimidin-5-yl]-3-chloro-2-fluorophenol

Steps 1-4 are same as in Example 62.

### Step-5: Synthesis of 5-(4-amino-7-(2,3-dihydro-M-inden-2-yl)-7H-pyrrolo[2,3-d]pyrimidin-5-yl)-3-chloro-2-fluorophenol

To a solution of 5-bromo-7-(2,3-dihydro-1H-inden-2-yl)-7H-pyrrolo[2,3-d]pyrimidin-4-amine (300 mg, 0.918 mmol) in DMF (5 mL) was added 3-chloro-4-fluoro-5-hydroxyphenylboronic acid (260 mg, 1.37 mmol) at RT. Then, Na₂CO₃ (290 mg, 2.73 mmol) dissolved in water (5 mL) was added to the reaction mixture followed by addition of Pd(PPh₃)₄ (73.6 mg, 0.0637 mmol) at RT and the resultant reaction mixture was heated at 80° C. for 3 h. The reaction was monitored by TLC and by LCMS. After completion of reaction, the reaction mixture was diluted with water (30 mL) and extracted with EtOAc (3×30 mL). The combined organic layers were washed with brine (50 mL), dried over sodium sulfate and concentrated. The crude compound was purified by preparative HPLC to obtain 5-(4-amino-7-(2,3-dihydro-1H-inden-2-yl)-7H-pyrrolo[2,3-d]pyrimidin-5-yl)-3-chloro-2-fluorophenol (80 mg) as an off-white solid. To this was added ethanolic HCl (10 mL) and stirred for 30 min at RT. The reaction mixture was then concentrated under reduced pressure and lyophilized to obtain 5-(4-amino-7-(2,3-dihydro-1H-inden-2-yl)-7H-pyrrolo[2,3-d]pyrimidin-5-yl)-3-chloro-2-fluorophenol (82.6 mg) as the HCl salt (off-white solid). ¹HNMR (400 MHz, Methanol-d4) δ (ppm): 8.35 (s, 1H), 7.31 (d, J=4.7 Hz, 2H), 7.28-7.20 (m, 2H), 6.97 (dd, J=5.9, 2.2 Hz, 1H), 6.91 (dd, J=7.4, 2.2 Hz, 1H), 5.76 (ddd, J=13.5, 7.7, 5.8 Hz, 1H), 3.58 (dd, J=16.2, 7.7 Hz, 2H), 3.37 (dd, J=16.2, 5.8 Hz, 2H).

### Example 111: Preparation of Compound No. 111

### Synthesis of 4-[4-amino-1-(2,3-dihydro-1H-inden-2-yl)-1H-pyrazolo[3,4-d]pyrimidin-3-yl]-2-hydroxybenzamide

Steps 1-6 are same as in Example 3.

### Step-7: Synthesis of 4-(4-amino-1-(2,3-dihydro-1H-inden-2-yl)-1H-pyrazolo[3,4-d]pyrimidin-3-yl)-2-hydroxybenzamide

To a solution of 4-(4-amino-1-(2,3-dihydro-1H-inden-2-yl)-1H-pyrazolo[3,4-d]pyrimidin-3-yl)-2-hydroxybenzonitrile (18 mg, 0.05 mmol) in tert-butanol (3 mL) was added KOH (8 mg, 0.15 mmol). The resultant reaction mixture was heated in a reagent bottle at 80° C. for 4h. The progress of the reaction was monitored by TLC and LCMS. After completion of reaction, solvent was evaporated and crude product which was purified by reverse phase preparative HPLC to afford 4-(4-amino-1-(2,3-dihydro-1H-inden-2-yl)-1H-pyrazolo[3,4-d]pyrimidin-3-yl)-2-hydroxybenzamide (5 mg) as a white solid. ¹HNMR (400 MHz, Methanol-d4) δ (ppm): 8.41 (s, 1H), 7.96 (d, J=7.9 Hz, 1H), 7.30-7.25 (m, 1H), 7.21 (d, J=7.4 Hz, 1H), 5.93-5.81 (m, 1H), 3.62 (dd, J=15.9, 7.5 Hz, 1H), 3.53 (dd, J=15.9, 8.4 Hz, 2H). LCMS: 387.1 (M+1).

### Example 112: Preparation of Compound No. 112

### Synthesis of 5-[4-amino-1-(2,3-dihydro-1H-inden-2-yl)-1H-pyrazolo[4,3-d]pyridin-3-yl]-2-chlorophenol

### Step-1: Synthesis of 2,4-dichloronicotinaldehyde

To a stirred solution of THF (200 mL) was added n-BuLi (1.6M in hexane, 46.4 mL, 74.32 mmol) dropwise at −78° C. Then, DIPEA (11.3 mL, 81.08 mmol) was added to the reaction mixture slowly at −78° C. and the temperature of the reaction mixture was gradually raised to 0° C. over a period of 1 h. Then 2, 4-dichloropyridine (10 g, 67.57 mmol) dissolved in THF (30 mL) was slowly added to the reaction mixture at −78° C. and the reaction mixture was allowed to stir at same temperature for 1h. Then, ethyl formate (10.91 mL, 135.14 mmol) dissolved in THF (20 mL) was slowly added to the reaction mixture at −78° C. and the resultant reaction mixture was stirred at −78° C. for 45 min. The reaction was monitored by TLC. After completion of reaction, the reaction mixture was quenched using saturated NH₄Cl solution (50 mL) at 0° C. slowly. The aq. layer was then extracted with EtOAc (2×300 mL). The combined organic layers were washed with water (400 mL), brine (100 mL), dried over Na₂SO₄ and concentrated to afford 2,4-dichloronicotinaldehyde (8.5 g) as a brown solid, which was used for next-step without any further purification.

### Step-2: Synthesis of 4-chloro-M-pyrazolo[4,3-c]pyridine

To a stirred solution of 2,4-dichloronicotinaldehyde (8.5 g, 48.29 mmol) in DME (60 mL) was added hydrazine hydrate (9.38 mL, 193.18 mmol) at 0° C. slowly and the resultant reaction mixture was heated at 75° C. for 16 h. The reaction was monitored by TLC. After completion of reaction, the reaction mixture was quenched using ice-cold water (200 mL). The aq. layer was then extracted with EtOAc (2×300 mL). The combined organic layers were washed with water (400 mL), brine (100 mL), dried over Na₂SO₄ and concentrated. The crude compound was purified by silica-gel (230-400) column chromatography, compound eluting at 20% EtOAc/hexane to afford 4-chloro-1H-pyrazolo[4,3-c]pyridine (1.35 g) as a light brown solid.

### Step-3: Synthesis of N-(4-methoxybenzyl)-1H-pyrazolo[4,3-c]pyridin-4-amine

To 4-chloro-1H-pyrazolo[4,3-c]pyridine (1.3 g, 8.46 mmol) was added (4-methoxyphenyl) methanamine (5 mL) and the resultant reaction mixture was heated in a closed reaction vessel at 150° C. for 6 h. The reaction was monitored by TLC. After completion of reaction, the reaction mixture was diluted with EtOAc (500 mL). The organic layer was washed with saturated NH₄Cl solution (12×50 mL), water (2×200 mL), brine (150 mL), dried over Na₂SO₄ and concentrated. The crude compound was purified by silica-gel (100-200) column chromatography, compound eluting at 40% EtOAc/hexane to afford N-(4-methoxybenzyl)-1H-pyrazolo[4,3-c]pyridin-4-amine (525 mg) as a light brown solid.

### Step-4: Synthesis of 1H-pyrazolo[4,3-c]pyridin-4-amine

To N-(4-Methoxybenzyl)-1H-pyrazolo[4,3-c]pyridin-4-amine (520 mg, 2.04 mmol) in DCM (2.5 mL) was added TFA (2.5 mL) at RT and the resultant reaction mixture was heated at 60° C. for 1 h. The reaction was monitored by TLC. After completion of reaction, the reaction mixture was cooled to RT and concentrated under reduced pressure. The crude residue was basified using saturated NaHCO₃ solution (100 mL) and extracted with EtOAc (22×50 mL). The combined organic layers were washed with water (100 mL), brine (100 mL), dried over Na₂SO₄ and concentrated. The crude compound was purified by silica-gel (100-200) column chromatography, compound eluting at 50% EtOAc/hexane to afford 1H-pyrazolo[4,3-c]pyridin-4-amine (200 mg) as a brown sticky solid.

### Step-5: Synthesis of 3-bromo-M-pyrazolo[4,3-c]pyridin-4-amine

To a stirred solution of 1H-pyrazolo[4,3-c]pyridin-4-amine (150 mg, 0.59 mmol) in glacial acetic acid (5 mL) was added bromine (104 mg, 0.65 mmol) at RT slowly and the resultant reaction mixture was stirred at RT for 10 min. The reaction was monitored by TLC. After completion of reaction, the reaction mixture was quenched with ice-cold water (10 mL) and extracted with EtOAc (2×100 mL). The combined organic layers were washed with saturated NaHCO₃ (100 mL) solution, water (80 mL), brine (50 mL), dried over Na₂SO₄ and concentrated to afford 3-bromo-1H-pyrazolo[4,3-c]pyridin-4-amine (140 mg) as a light brown sticky solid, which was taken to the next step without further purification.

### Step-6: Synthesis of 3-bromo-1-(2,3-dihydro-1H-inden-2-yl)-1H-pyrazolo[4,3-c]pyridin-4-amine

To a stirred solution of 3-bromo-1H-pyrazolo[4,3-c]pyridin-4-amine (130 mg, 0.610 mmol) in DMF (6 mL) was added Cs₂CO₃ (397.6 mg, 1.22 mmol) at RT and the reaction mixture was allowed to stir for 45 min. Then, TBAI (45.08 mg, 0.122 mmol) and 2, 3-dihydro-1H-inden-2-yl methanesulfonate (259.07 mg, 1.22 mmol) were successively added to the reaction mixture and the resultant reaction mixture was heated at 80° C. for 1.5 h. The reaction was monitored by TLC. After completion of reaction, the reaction mixture was diluted with EtOAc (200 mL). The organic layer was washed with water (2×50 mL), brine (50 mL), dried over Na₂SO₄ and concentrated. The crude compound was triturated with ether-pentane (1:3) to afford 3-bromo-1-(2, 3-dihydro-1H-inden-2-yl)-1H-pyrazolo[4,3-c]pyridin-4-amine (60 mg) as a brown sticky solid.

### Step-7: Synthesis of 5-(4-amino-1-(2,3-dihydro-1H-inden-2-yl)-1H-pyrazolo[4,3-c]pyridin-3-yl)-2-chlorophenol

To a solution of 3-bromo-1-(2, 3-dihydro-1H-inden-2-yl)-1H-pyrazolo[4,3-c]pyridin-4-amine (60 mg, 0.182 mmol) in DMF (3 mL) was added 4-chloro-3-hydroxyphenylboronic acid (47.12 mg, 0.273 mmol) at RT. Then, Na₂CO₃ (57.94 mg, 0.546 mmol) dissolved in water (3 mL) was added to the reaction mixture followed by addition of Pd(PPh₃)₄ (14.74 mg, 0.012 mmol) at RT and the resultant reaction mixture was heated at 80° C. for 2 h. The reaction was monitored by TLC and by LCMS. After completion of reaction, the reaction mixture was diluted with water (25 mL) and extracted with EtOAc (2×50 mL). The combined organic layers were washed with water (2×20 mL), brine (20 mL), dried over sodium sulfate and concentrated. The crude compound was purified by combiflash column chromatography to obtain 5-(4-amino-1-(2, 3-dihydro-1H-inden-2-yl)-1H-pyrazolo[4,3-c]pyridin-3-yl)-2-chlorophenol (1.02 mg) as the HCl salt (off-white solid). ¹HNMR (400 MHz, Methanol-d4) δ (ppm): 8.75 (s, 1H), 7.59 (d, J=2.0 Hz, 1H), 7.54 (s, 1H), 7.40-7.30 (m, 4H), 7.30-7.22 (m, 2H), 5.60 (tt, J=7.4, 4.9 Hz, 1H), 3.69 (dd, J=16.4, 7.5 Hz, 2H), 3.53 (dd, J=16.4, 4.9 Hz, 2H).

### Example 113: Preparation of Compound No. 113

### Synthesis of 5-[3,4-difluoro-5-(methylamino)phenyl]-7-(2,3-dihydro-1H-inden-2-yl)-7H-pyrrolo[2,3-d]pyrimidin-4-amine

To a solution of 2,3-difluoro-N-methyl-5-(4,4,5,5-tetramethyl-1,3,2-dioxaborolan-2-yl)aniline (98 mg, 0.365 mmol) in DMF (5 mL) was added a solution of sodium carbonate (96.6 mg, 0.912 mmol) in water (5 mL) and the reaction mixture was degassed under nitrogen for 15 min, followed by addition of 5-bromo-7-indan-2-yl-pyrrolo[2,3-d]pyrimidin-4-amine (100 mg, 0.304 mmol) and Pd(PPh₃)₄ (24.6 mg, 0.02128 mmol) and the resultant reaction mixture was allowed to heat overnight at 100° C. After completion of reaction, the reaction mixture was cooled to RT, diluted with EtOAc (200 mL), washed with water (80 mL), brine (90 mL). The combined organic layer was dried over anhydrous sodium sulfate, concentrated under reduced pressure to obtain a crude product, which was purified by reverse HPLC to give 5-[3,4-difluoro-5-(methylamino)phenyl]-7-indan-2-yl-pyrrolo[2,3-d]pyrimidin-4-amine (20 mg), which was mixed with ethanolic HCl and kept for 1h to give 5-[3,4-difluoro-5-(methylamino)phenyl]-7-indan-2-yl-pyrrolo[2,3-d]pyrimidin-4-amine (22 mg) as the HCl salt (off-white solid). ¹HNMR (400 MHz, Methanol-d4) δ (ppm): 8.35 (s, 1H), 7.36-7.27 (m, 3H), 7.24 (dd, J=5.5, 3.3 Hz, 2H), 6.51 (t, J=9.0 Hz, 2H), 5.76 (q, J=6.9 Hz, 1H), 3.59 (dd, J=16.2, 7.8 Hz, 2H), 3.39 (dd, J=16.1, 5.9 Hz, 2H), 2.84 (s, 3H).

### Example 114: Preparation of Compound Nos. 114, 114a, and 114b

### Synthesis of 5-[4-amino-7-(1,2,3,4-tetrahydronaphthalen-2-yl)-7H-pyrrolo[2,3-d]pyrimidin-5-yl]-2,3-difluorophenol

To a suspension of (3,4-difluoro-5-hydroxy-phenyl)boronic acid (113.5 mg, 0.656 mmol) in DMF (4 mL) was added a solution of sodium carbonate (139 mg, 1.3119 mmol) in water (4 mL) and the reaction mixture was degassed under nitrogen for 15 min. After 15 min, 5-bromo-7-tetralin-2-yl-pyrrolo[2,3-d]pyrimidin-4-amine (150 mg, 0.437 mmol) and Pd(PPh₃)₄ (35 mg, 0.031 mmol) were added and reaction mixture allowed to heat overnight at 100° C. After completion of reaction, the reaction mixture was cooled to RT, diluted with EtOAc (100 mL), washed with water (2×50 mL) and brine (50 mL). The combined organic layer was dried over anhydrous sodium sulfate, concentrated under reduced pressure to give a crude product, which was purified by reverse HPLC to give 5-(4-amino-7-tetralin-2-yl-pyrrolo[2,3-d]pyrimidin-5-yl)-2,3-difluoro-phenol (15 mg), which was further mixed with ethanolic —HCl and kept for 1 h to obtain 5-(4-amino-7-tetralin-2-yl-pyrrolo[2,3-d]pyrimidin-5-yl)-2,3-difluoro-phenol (18 mg) as the HCl salt (off-white solid). ¹HNMR (400 MHz, Methanol-d4) δ (ppm): 8.32 (s, 1H), 7.57 (s, 1H), 7.15 (d, J=14.1 Hz, 4H), 6.86 (dd, J=8.1, 5.6 Hz, 2H), 5.20 (s, 1H), 3.05 (ddd, J=22.5, 13.9, 9.5 Hz, 2H), 2.50-2.35 (m, 2H), 2.31 (s, 2H). Separation by chiral HPLC affords Compound Nos. 114a and 114b.

### Example 115: Preparation of Compound No. 115

### Synthesis of 5-[4-amino-7-(2,3-dihydro-1H-inden-2-yl)-7H-pyrrolo[2,3-d]pyrimidin-5-yl]-2-fluoro-3-hydroxybenzonitrile

To a suspension of (3-cyano-4-fluoro-5-hydroxy-phenyl)boronic acid (248 mg, 1.365 mmol) in DMF (5 mL) was added a solution of sodium carbonate (290 mg, 2.73 mmol) in water (5 mL) and the reaction mixture was degassed under nitrogen for 15 min. After 15 min, 5-bromo-7-indan-2-yl-pyrrolo[2,3-d]pyrimidin-4-amine (300 mg, 0.91 mmol) and Pd(PPh₃)₄ (73.6 mg, 0.0637 mmol) was added to reaction mixture and allowed to heat at 100° C. overnight. After completion of reaction, the reaction mixture was allowed to cool at RT, diluted with EtOAc (100 mL), washed with water (2×50 mL) and brine (50 mL). The combined organic layer was dried over anhydrous sodium sulfate, concentrated under reduced pressure to give a crude product, which was purified over reverse HPLC to give 5-(4-amino-7-indan-2-yl-pyrrolo[2,3-d]pyrimidin-5-yl)-2-fluoro-3-hydroxy-benzonitrile (45 mg). The product was further allowed to mix with ethanolic HCl and lyophilized to give 5-(4-amino-7-indan-2-yl-pyrrolo[2,3-d]pyrimidin-5-yl)-2-fluoro-3-hydroxy-benzonitrile (48 mg) as the HCl salt (off-white solid). ¹HNMR (400 MHz, Methanol-d4) δ (ppm): 8.36 (s, 1H), 7.36 (s, 1H), 7.34-7.27 (m, 2H), 7.23 (dtd, J=9.2, 5.0, 2.1 Hz, 4H), 5.77 (p, J=7.2, 6.6 Hz, 1H), 3.59 (dd, J=16.1, 7.8 Hz, 2H), 3.46-3.35 (m, 2H).

### Example 116: Preparation of Compound No. 116

### Synthesis of 5-[4-amino-7-(2,3-dihydro-1H-inden-2-yl)-7H-pyrrolo[2,3-d]pyrimidin-5-yl]-2-fluoro-3-hydroxybenzamide

To a stirred solution of 5-(4-amino-7-indan-2-yl-pyrrolo[2,3-d]pyrimidin-5-yl)-2-fluoro-3-hydroxy-benzonitrile (75 mg, 0.194 mmol) in t-butanol (10 mL) was added potassium hydroxide (54 mg, 0.97 mmol) and the reaction mixture was heated at 100° C. for 5 h. After completion of reaction, the reaction mixture was cooled to RT, concentrated under reduced pressure, diluted with EtOAc (300 mL), washed with water (100 mL), dried over anhydrous sodium sulfate, concentrated under reduced pressure to give a crude product, which was triturated with acetone (5 mL) to give 5-(4-amino-7-indan-2-yl-pyrrolo[2,3-d]pyrimidin-5-yl)-2-fluoro-3-hydroxy-benzamide (45 mg). The product was mixed with ethanolic HCl and lyophilized to obtain 5-(4-amino-7-indan-2-yl-pyrrolo[2,3-d]pyrimidin-5-yl)-2-fluoro-3-hydroxy-benzamide (48 mg) as the HCl salt (off-white solid). ¹HNMR (400 MHz, Methanol-d4) δ (ppm): 8.34 (s, 1H), 7.34-7.26 (m, 3H), 7.23 (ddd, J=8.1, 5.6, 2.7 Hz, 3H), 7.11 (dd, J=7.9, 2.3 Hz, 1H), 5.76 (q, J=6.7 Hz, 1H), 3.59 (dd, J=16.2, 7.7 Hz, 2H), 3.38 (d, J=5.6 Hz, 1H), 3.34 (d, J=5.6 Hz, 1H).

### Example 117: Preparation of Compound No. 117

### Synthesis of 5-[4-amino-1-(2,3-dihydro-1H-inden-2-yl)-1H-pyrazolo[3,4-d]pyrimidin-3-yl]-3-chloro-2-fluorophenol

To a solution of 1-(2,3-dihydro-1H-inden-2-yl)-3-iodo-1H-pyrazolo[3,4-d]pyrimidin-4-amine (300 mg, 0.795 mmol) in DMF (3 mL) was added 3-chloro-4-fluoro-5-hydroxyphenylboronic acid (227 mg, 1.19 mmol) at RT. Then, Na₂CO₃ (252.8 mg, 2.38 mmol) dissolved in water (3 mL) was added to the reaction mixture followed by addition of Pd(PPh₃)₄ (64 mg, 0.05 mmol) at RT and the resultant reaction mixture was heated at 85° C. for 2 h. The reaction was monitored by TLC and by LCMS. After completion of reaction, the reaction mixture was diluted with water (25 mL) and extracted with EtOAc (100 mL×3). The combined organic layers were washed with water (200 mL), brine (200 mL), dried over sodium sulfate and concentrated. The crude compound was purified by preparative HPLC to 5-(4-amino-1-(2,3-dihydro-1H-inden-2-yl)-1H-pyrazolo[3,4-d]pyrimidin-3-yl)-3-chloro-2-fluorophenol (107 mg) as an off-white solid. To this was added ethanolic HCl (3 mL) and stirred for 30 min at RT. The reaction mixture was then concentrated under reduced pressure and lyophilized to obtain 5-(4-amino-1-(2,3-dihydro-1H-inden-2-yl)-1H-pyrazolo[3,4-d]pyrimidin-3-yl)-3-chloro-2-fluorophenol (111.8 mg) as the HCl salt (light brown solid). ¹HNMR (400 MHz, Methanol-d4) δ (ppm): 8.42 (s, 1H), 7.30-7.23 (m, 2H), 7.26-7.12 (m, 4H), 5.87 (q, J=7.9 Hz, 1H), 3.57 (m, 4H).

### Example 118: Preparation of Compound No. 118

### Synthesis of 5-[4-amino-1-(2,3-dihydro-1H-inden-2-yl)-1H-pyrazolo[3,4-d]pyrimidin-3-yl]-2,3-dichlorophenol

To a solution of 1-(2,3-dihydro-1H-inden-2-yl)-3-iodo-1H-pyrazolo[3,4-d]pyrimidin-4-amine (300 mg, 0.795 mmol) in DMF (3 mL) was added 3,4-dichloro-5-hydroxyphenylboronic acid (245 mg, 1.19 mmol) at RT. Then, Na₂CO₃ (252.8 mg, 2.38 mmol) dissolved in water (3 mL) was added to the reaction mixture followed by addition of Pd(PPh₃)₄ (64 mg, 0.05 mmol) at RT and the resultant reaction mixture was heated at 85° C. for 2 h. The reaction was monitored by TLC and by LCMS. After completion of reaction, the reaction mixture was diluted with water (25 mL) and extracted with EtOAc (3×100 mL). The combined organic layers were washed with water (200 mL), brine (200 mL), dried over sodium sulfate and concentrated. The crude compound was purified by preparative HPLC to 5-(4-amino-1-(2,3-dihydro-1H-inden-2-yl)-1H-pyrazolo[3,4-d]pyrimidin-3-yl)-2,3-dichlorophenol (85 mg) as an off-white solid. To this was added ethanolic HCl (3 mL) and stirred for 30 min at RT. The reaction mixture was then concentrated under reduced pressure and lyophilized to obtain 5-(4-amino-1-(2,3-dihydro-1H-inden-2-yl)-1H-pyrazolo[3,4-d]pyrimidin-3-yl)-2,3-dichlorophenol (89.3 mg) as the HCl salt (light brown solid). ¹HNMR (400 MHz, Methanol-d4) δ (ppm): 8.43 (s, 1H), 7.34-7.13 (m, 6H), 5.87 (p, J=7.8 Hz, 1H), 3.57 (qd, J=15.9, 7.9 Hz, 4H).

### Example 119: Preparation of Compound Nos. 119, 119a and 119b

### Synthesis of 5-[4-amino-1-(1,2,3,4-tetrahydronaphthalen-2-yl)-1H-pyrazolo[3,4-d]pyrimidin-3-yl]-2,3-difluorophenol

To a solution of 3-iodo-1-(1,2,3,4-tetrahydronaphthalen-2-yl)-1H-pyrazolo[3,4-d]pyrimidin-4-amine (300 mg, 0.76 mmol) in DMF (4 mL) was added 3,4-difluoro-5-hydroxyphenylboronic acid (267 mg, 1.53 mmol) at RT. Then, Na₂CO₃ (162.1 mg, 1.53 mmol) dissolved in water (4 mL) was added to the reaction mixture followed by addition of Pd(PPh₃)₄ (61 mg, 0.05 mmol) at RT and the resultant reaction mixture was heated at 85° C. for 2 h. The reaction was monitored by TLC and by LCMS. After completion of reaction, the reaction mixture was diluted with water (25 mL) and extracted with EtOAc (3×100 mL). The combined organic layers were washed with water (200 mL), brine (200 mL), dried over sodium sulfate and concentrated. The crude compound was purified by preparative HPLC to give 5-(4-amino-1-(1,2,3,4-tetrahydronaphthalen-2-yl)-1H-pyrazolo[3,4-d]pyrimidin-3-yl)-2,3-difluorophenol (76 mg) as an off-white solid. To this was added ethanolic HCl (3 mL) and stirred for 30 min at RT. The reaction mixture was then concentrated under reduced pressure and lyophilized to obtain 5-(4-amino-1-(1,2,3,4-tetrahydronaphthalen-2-yl)-1H-pyrazolo[3,4-d]pyrimidin-3-yl)-2,3-difluorophenol (80 mg) as the HCl salt (light brown solid). ¹HNMR (400 MHz, Methanol-d4) δ (ppm): 8.41 (s, 1H), 7.22-7.10 (m, 2H), 7.07 (d, J=7.1 Hz, 1H), 5.27 (tt, J=11.2, 5.2 Hz, 1H), 3.54 (dd, J=16.1, 11.0 Hz, 1H), 3.22 (dd, J=16.2, 5.4 Hz, 1H), 3.09 (dd, J=10.5, 5.8 Hz, 2H), 2.49 (qd, J=11.6, 11.2, 6.9 Hz, 1H), 2.30 (d, J=11.9 Hz, 1H). Separation by chiral HPLC affords Compound Nos. 119a and 119b.

### Example 120: Preparation of Compound No. 120

### Synthesis of 5-[4-amino-1-(2,3-dihydro-1H-inden-2-yl)-1H-pyrazolo[3,4-d]pyrimidin-3-yl]-2-fluoro-3-hydroxybenzonitrile

To a solution of 1-(2,3-dihydro-1H-inden-2-yl)-3-iodo-1H-pyrazolo[3,4-d]pyrimidin-4-amine (280 mg, 0.742 mmol) in DMF (5 mL) was added 3-cyano-4-fluoro-5-hydroxyphenylboronic acid (201.47 mg, 1.11 mmol) at RT. Then, Na₂CO₃ (236.02 mg, 2.22 mmol) dissolved in water (5 mL) was added to the reaction mixture followed by addition of Pd(PPh₃)₄ (60.04 mg, 0.051 mmol) at RT and the resultant reaction mixture was heated at 90° C. for 16 h. The progress of the reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 5-(4-amino-1-(2,3-dihydro-1H-inden-2-yl)-1H-pyrazolo[3,4-d]pyrimidin-3-yl)-2-fluoro-3-hydroxybenzonitrile (120 mg) as an off-white solid. From this 20 mg of compound was taken and to it was added 4N-HCl in 1,4-dioxane (5 mL) and stirred for 30 min at RT. The reaction mixture was then concentrated under reduced pressure and lyophilized to obtain 5-(4-amino-1-(2,3-dihydro-1H-inden-2-yl)-1H-pyrazolo[3,4-d] pyrimidin-3-yl)-2-fluoro-3-hydroxybenzonitrile (22 mg) as the HCl salt (off-white solid). ¹HNMR (400 MHz, DMSO-d6) δ (ppm): 11.02 (s, 1H), 8.34 (s, 1H), 7.57-7.42 (m, 2H), 7.31-7.16 (m, 4H), 5.75 (p, J=7.9 Hz, 1H), 3.49 (dd, J=7.7, 2.5 Hz, 4H).

### Example 121: Preparation of Compound No. 121

### Synthesis of 1-(5-fluoro-2,3-dihydro-1H-inden-2-yl)-3-(8-fluoro-3,4-dihydro-2H-1,4-benzoxazin-6-yl)-1H-pyrazolo[3,4-d]pyrimidin-4-amine

To a solution of 2-(4-amino-3-(8-fluoro-3,4-dihydro-2H-benzo[b][1,4]oxazin-6-yl)-1H-pyrazolo[3,4-d]pyrimidin-1-yl)-6-fluoro-2,3-dihydro-1H-inden-1-one (86 mg, 0.198 mmol) in TFA (1.5 mL) was added triethylsilane (1.6 mL, 9.90 mmol) at RT and the resultant reaction mixture was heated at 100° C. for 6 h. The progress of the reaction was monitored by LCMS. After 6 h, the reaction mixture was cooled to RT, quenched using ice and then basified using saturated NaHCO₃ solution (50 mL). The aq. layer was then extracted using EtOAc (3×50 mL). The combined organic layers were washed with brine (50 mL), dried over sodium sulfate and concentrated. The crude compound was purified by preparative TLC to obtain 1-(5-fluoro-2,3-dihydro-1H-inden-2-yl)-3-(8-fluoro-3,4-dihydro-2H-benzo[b][1,4] oxazin-6-yl)-1H-pyrazolo[3,4-d]pyrimidin-4-amine (0.9 mg) as an off-white solid. To this was added ethanolic HCl (2 mL) and kept for 20 min at RT. The reaction mixture was then concentrated under reduced pressure and lyophilized to obtain 1-(5-fluoro-2,3-dihydro-1H-inden-2-yl)-3-(8-fluoro-3,4-dihydro-2H-benzo[b][1,4]oxazin-6-yl)-1H-pyrazolo[3,4-d] pyrimidin-4-amine (1 mg) as the HCl salt (off-white solid). ¹HNMR (400 MHz, Methanol-d4) δ (ppm): 8.41 (s, 1H), 7.30-7.20 (m, 1H), 7.01 (d, J=8.9 Hz, 1H), 6.93 (d, J=8.7 Hz, 1H), 6.68 (d, J=9.8 Hz, 2H), 5.88 (t, J=7.8 Hz, 1H), 4.28 (t, J=4.4 Hz, 2H), 3.54 (dt, J=23.4, 7.4 Hz, 4H), 3.43 (t, J=4.4 Hz, 2H).

### Example 122: Preparation of Compound Nos. 122, 122a and 122b

### Synthesis of 3-(3-amino-4,5-difluorophenyl)-1-(1,2,3,4-tetrahydronaphthalen-2-yl)-1H-pyrazolo[3,4-d]pyrimidin-4-amine

To a solution of 3-iodo-1-(1,2,3,4-tetrahydronaphthalen-2-yl)-1H-pyrazolo[3,4-d]pyrimidin-4-amine (200 mg, 0.511 mmol) in DMF (4 mL) was added 3-amino-4,5-difluorophenylboronic acid (195.6 mg, 0.767 mmol) at RT. Then, Na₂CO₃ (162.5 mg, 1.53 mmol) dissolved in water (4 mL) was added to the reaction mixture followed by addition of Pd(PPh₃)₄ (41.37 mg, 0.035 mmol) at RT and the resultant reaction mixture was heated at 90° C. for 2 h. The progress of the reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 3-(3-amino-4,5-difluorophenyl)-1-(1,2,3,4-tetrahydro-naphthalen-2-yl)-1H-pyrazolo[3,4-d]pyrimidin-4-amine (103 mg) as an off-white solid. To this was added 4N-HCl in 1,4-dioxane (10 mL) and stirred for 30 min at RT. The reaction mixture was then concentrated under reduced pressure and lyophilized to obtain 3-(3-amino-4,5-difluorophenyl)-1-(1,2,3,4-tetrahydronaphthalen-2-yl)-1H-pyrazolo[3,4-d] pyrimidin-4-amine (105 mg) as the HCl salt (off-white solid). ¹HNMR (400 MHz, Methanol-d4) δ (ppm): 8.40 (s, 1H), 7.19-7.09 (m, 4H), 6.93 (d, J=7.3 Hz, 1H), 6.84-6.75 (m, 1H), 5.26 (tt, J=10.7, 4.9 Hz, 1H), 3.62-3.48 (m, 2H), 3.21 (dd, J=16.1, 5.2 Hz, 1H), 3.09 (dd, J=10.7, 5.7 Hz, 1H), 2.49 (qd, J=11.6, 6.8 Hz, 1H), 2.29 (d, J=12.7 Hz, 1H). Separation by chiral HPLC affords Compound Nos. 122a and 122b.

### Example 123: Preparation of Compound Nos. 123, 123a and 123b

### Synthesis of 5-[4-amino-1-(1,2,3,4-tetrahydronaphthalen-2-yl)-1H-pyrazolo[3,4-d]pyrimidin-3-yl]-2-fluoro-3-(trifluoromethyl)phenol

To a solution of 3-iodo-1-(1,2,3,4-tetrahydronaphthalen-2-yl)-1H-pyrazolo[3,4-d]pyrimidin-4-amine (250 mg, 0.639 mmol) in DMF (4.5 mL) was added 4-fluoro-3-hydroxy-5-(trifluoromethyl)phenylboronic acid (214.75 mg, 0.959 mmol) at RT. Then, Na₂CO₃ (203.13 mg, 1.91 mmol) dissolved in water (4.5 mL) was added to the reaction mixture followed by addition of Pd(PPh₃)₄ (51.71 mg, 0.044 mmol) at RT and the resultant reaction mixture was heated at 90° C. for 2 h. The progress of the reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude compound was purified by preparative HPLC to obtain 5-(4-amino-1-(1,2,3,4-tetrahydro-naphthalen-2-yl)-1H-pyrazolo[3,4-d]pyrimidin-3-yl)-2-fluoro-3-(trifluoromethyl) phenol (85 mg) as an off-white solid. To this was added 4N-HCl in 1,4-dioxane (10 mL) and stirred for 30 min at RT. The reaction mixture was then concentrated under reduced pressure and triturated with diethyl ether (2×5 mL) to obtain 5-(4-amino-1-(1,2,3,4-tetrahydronaphthalen-2-yl)-1H-pyrazolo[3,4-d]pyrimidin-3-yl)-2-fluoro-3-trifluoromethyl)phenol (86 mg) as the HCl salt (off-white solid). ¹HNMR (400 MHz, Methanol-d4) δ (ppm): 8.43 (s, 1H), 7.51 (d, J=7.8 Hz, 1H), 7.41 (d, J=4.3 Hz, 1H), 7.34-6.94 (m, 4H), 5.28 (td, J=11.2, 5.8 Hz, 1H), 3.55 (dd, J=16.1, 10.9 Hz, 2H), 3.23 (dd, J=16.1, 5.3 Hz, 1H), 3.09 (dd, J=10.6, 5.7 Hz, 1H), 2.50 (qd, J=11.5, 6.8 Hz, 1H), 2.31 (d, J=11.6 Hz, 1H). Separation by chiral HPLC affords Compound Nos. 123a and 123b.

### Example 124: Preparation of Compound Nos. 124, 124a and 124b

### Synthesis of 3-(8-fluoro-3,4-dihydro-2H-1,4-benzoxazin-6-yl)-1-(1,2,3,4-tetrahydronaphthalen-2-yl)-1H-pyrazolo[3,4-d]pyrimidin-4-amine

To a solution of 3-iodo-1-(1,2,3,4-tetrahydronaphthalen-2-yl)-1H-pyrazolo[3,4-d]pyrimidin-4-amine (225 mg, 0.575 mmol) in DMF (4.5 mL) was added 8-fluoro-3,4-dihydro-2H-benzo[b][1,4]oxazin-6-ylboronic acid (286.6 mg, 0.978 mmol) at RT. Then, Na₂CO₃ (182.8 mg, 1.72 mmol) dissolved in water (4.5 mL) was added to the reaction mixture followed by addition of Pd(PPh₃)₄ (46.5 mg, 0.046 mmol) at RT and the resultant reaction mixture was heated at 90° C. for 2 h. The progress of the reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over anhydrous sodium sulfate and concentrated under low pressure. The crude compound was purified by preparative HPLC to obtain 3-(8-fluoro-3,4-dihydro-2H-benzo[b][1,4]oxazin-6-yl)-1-(1,2,3,4-tetrahydronaphthalen-2-yl)-1H-pyrazolo[3,4-d]pyrimidin-4-amine (111 mg) as an off-white solid. To this was added 4N-HCl in 1,4-dioxane (10 mL) and stirred for 30 min at RT. The reaction mixture was then concentrated under reduced pressure and lyophilized to obtain 3-(8-fluoro-3,4-dihydro-2H-benzo[b][1,4]oxazin-6-yl)-1-(1,2,3,4-tetrahydronaphthalen-2-yl)-1H-pyrazol[3,4-d]pyrimidin-4-amine (113 mg) as the HCl salt (off-white solid). ¹HNMR (400 MHz, Methanol-d4) δ (ppm): 8.38 (s, 1H), 7.18-7.09 (m, 4H), 6.72 (d, J=11.1 Hz, 2H), 5.25 (dt, J=10.8, 6.2 Hz, 1H), 4.30 (t, J=4.4 Hz, 2H), 3.62-3.48 (m, 1H), 3.45 (t, J=4.4 Hz, 2H), 3.20 (dd, J=16.0, 5.3 Hz, 1H), 3.09 (dd, J=10.5, 5.5 Hz, 2H), 2.56-2.41 (m, 1H), 2.29 (s, 1H). Separation by chiral HPLC affords Compound Nos. 124a and 124b.

### Example 125: Preparation of Compound No. 125

### Synthesis of 5-[4-amino-7-(2,3-dihydro-1H-inden-2-yl)pyrrolo[2,1-f][1,2,4]triazin-5-yl]-2,3-difluorophenol

### Step-1: Synthesis of 7-(1H-inden-2-yl)pyrrolo[2,1-f][1,2,4]triazin-4-amine

To a solution of compound 7-bromopyrrolo[2,1-f][1,2,4]triazin-4-amine (1.5 g, 7.0 mmol) in DME (20 mL), the pinacol ester of 2-indeneboronic acid (1.5 eq.), 2M aq. Na₂CO₃ (4.0 eq.) were added and degassed with N2 for 15 min. Then Pd(dppf)Cl₂.DCM (5 mol %) was added to the mixture and again degassed for 15 min. The solution was heated in at 100° C. for 1 h and then allowed to cool at RT. Then the mixture was diluted with 25 mL water and 25 mL EtOAc and extracted. Then the organic layer was washed with brine and dried over anhydrous sodium sulfate and concentrated. The crude gummy liquid was purified by column to afford 7-(1H-inden-2-yl)pyrrolo[2,1-f][1,2,4]triazin-4-amine (970 mg, 57% yield).

### Step-2: Synthesis of 7-(2,3-dihydro-1H-inden-2-yl)pyrrolo[2,1-f][1,2,4]triazin-4-amine

7-(1H-Inden-2-yl)pyrrolo[2,1-f][1,2,4]triazin-4-amine (1.4 g, 5.64 mmol) was dissolved in AcOH (100 mL), followed by catalytic addition of 10% Pd—C(10 mol %). Then the mixture was hydrogenated under and 80 psi for 6h. The progress of the reaction was monitored by TLC. After completion of reaction, the AcOH was evaporated and the residue was dissolved in EtOAc, neutralized with satd. NaHCO₃ and separated. The organic layer was washed again with water, brine and dried over anhydrous sodium sulfate and concentrated under reduced pressure to 7-(2,3-dihydro-1H-inden-2-yl)pyrrolo[2,1-f][1,2,4]triazin-4-amine (720 mg, 51% yield).

The above reaction was repeated with 970 mg scale to obtain 830 mg of compound 3 (85% yield). The same reaction was carried in 1080 mg scale to obtain 810 mg of product (74% yield).

### Step-3: Synthesis of 7-(2,3-dihydro-1H-inden-2-yl)-5-iodopyrrolo[2,1-f][1,2,4]triazin-4-amine

To a solution of 7-(2,3-dihydro-1H-inden-2-yl)pyrrolo[2,1-f][1,2,4]triazin-4-amine (720 mg, 2.88 mmol) in THF (10 mL), NIS (1.2 eq.) were added portion wise at RT and allowed to stir for 3h at the same temperature. The progress of the reaction was monitored by TLC. After complication of the reaction THF was evaporated in vacuum and water (30 mL) was added to it to get precipitation. The precipitated solid was filtered and washed with water. Then the crude product was purified by column chromatography to get the pure product.

### Step-4: Synthesis of 5-(4-amino-7-indan-2-yl-pyrrolo[2,1-f][1,2,4]triazin-5-yl)-2,3-difluoro-phenol

To a solution of 7-indan-2-yl-5-iodo-pyrrolo[2,1-f][1,2,4]triazin-4-amine (200 mg, 1.15 mmol) and 5-bromo-2,3-difluoro-phenol (288 mg, 0.76 mmol) in 3 mL DMF, a aqueous solution of Na₂CO₃ (243 mg in 3 mL water, 2.29 mmol) were added at RT. Then the mixture was degassed with N2 for 10 min. followed by addition of tetrakis(triphenylphosphine) palladium(0) (88 mg, 0.08 mmol) and the mixture was heated at 90° C. for 3h. The reaction mixture was allowed to cool at RT and diluted with 25 mL EtOAc. The organic layer was washed with water (3×20 mL), brine and finally dried over Na₂SO₄. Finally the organic solution was concentrated under reduced pressure and the desire crude product compound was purified by HPLC as a white powder. Yield: 110 mg, 38%. 5-(4-Amino-7-indan-2-yl-pyrrolo[2,1-f][1,2,4]triazin-5-yl)-2,3-difluoro-phenol (59 mg) was dissolved in 10 mL of ethanolic HCl and the solution was evaporated under reduced pressure to get the corresponding hydrochloride salt. Yield: 61 mg, 99%. ¹HNMR (400 MHz, Methanol-d4): δ (ppm): 8.06 (s, 1H), 7.26 (dd, J=5.4, 3.3 Hz, 2H), 7.17 (dd, J=5.6, 3.2 Hz, 2H), 6.87 (dd, J=10.5, 6.5 Hz, 2H), 6.75 (s, 1H), 4.23 (p, J=8.0 Hz, 1H), 3.48 (dd, J=15.5, 8.2 Hz, 2H), 3.20 (dd, J=15.5, 8.0 Hz, 2H). LCMS (M+1): 379.

### Example 126: Preparation of Compound Nos. 126, 126a and 126b

### Synthesis of 5-(4-amino-1-{5H,6H,7H-cyclopenta[b]pyridin-5-yl}-1H-pyrazolo[3,4-d]pyrimidin-3-yl)-2,3-difluorophenol

To a stirred solution of 5-(4-amino-1H-pyrazolo [3,4-d] pyrimidin-3-yl)-2, 3-difluorophenol (100 mg, 0.380 mmol) in DMF (8 mL) was added cesium carbonate (247 mg, 0.76 mmol). The reaction mixture was allowed to stir at RT for 45 min. and then a solution of 5-chloro-6,7-dihydro-5H-cyclopenta[b]pyridine (116 mg, 0.760 mmol) in DMF (2 mL) and TBAI (14 mg, 0.038 mmol) were added to reaction mixture and heated at 80° C. for 1 h. The progress of the reaction was monitored by TLC. After completion of the reaction, the mixture was allowed to cool at RT followed by addition of water (25 mL) and extracted with EtOAc (2×100 mL). The organic layer was washed with water (30 mL), brine (30 mL), dried over sodium sulfate and concentrated under reduced pressure. The crude product was purified by reverse phase HPLC to obtain 5-(4-amino-1-(6, 7-dihydro-5H-cyclopenta[b]pyridin-5-yl)-1H-pyrazolo [3,4-d] pyrimidin-3-yl)-2, 3-difluorophenol (14 mg) as a free base. The product was further dissolved in 2 mL of dioxane-HCl (2 mL) and kept for 20 min at RT. The reaction mixture was then concentrated under reduced pressure and lyophilized to obtain 5-(4-amino-1-(6,7-dihydro-5H-cyclopenta[b]pyridin-5-yl)-1H-pyrazolo[3,4-d]pyrimidin-3-yl)-2,3-difluorophenol (14.9 mg) as the corresponding HCl salt (off-white solid). ¹HNMR (400 MHz, Methanol-d4) δ (ppm): 8.83-8.76 (m, 1H), 8.61 (d, J=7.7 Hz, 1H), 8.45 (s, 1H), 7.97-7.88 (m, 1H), 7.52 (d, J=6.6 Hz, 1H), 7.40-7.31 (m, 1H), 6.21 (dd, J=7.0, 4.2 Hz, 1H), 3.60-3.48 (m, 1H), 3.38 (ddd, J=12.1, 8.7, 4.3 Hz, 1H), 2.95 (dq, J=14.2, 6.8 Hz, 1H), 2.51 (ddt, J=14.1, 9.4, 5.0 Hz, 1H). Separation by chiral HPLC affords Compound Nos. 126a and 126b.

### Example 127: Preparation of Compound Nos. 127, 127a and 127b

### Synthesis of 2-[4-amino-3-(3,4-difluoro-5-hydroxyphenyl)-1H-pyrazolo[3,4-d]pyrimidin-1-yl]-5-fluoro-2,3-dihydro-1H-inden-1-one

To 2-(4-amino-3-(3,4-difluoro-5-hydroxyphenyl)-1H-pyrazolo[3,4-d]pyrimidin-1-yl)-5-fluoro-2,3-dihydro-1H-inden-1-one (170 mg, 0.413 mmol) was added TFA (2.5 mL) at 0° C. slowly followed by addition of triethylsilane (0.66 mL, 7.11 mmol) and the resultant reaction mixture was heated at 100° C. for 16 h. The progress of the reaction was monitored by LCMS. After 16 h, the reaction mixture was cooled to RT and concentrated under reduced pressure. The residue obtained was basified using saturated NaHCO₃ solution (30 mL) and then extracted with EtOAc (2×200 mL). The combined organic layers were washed with water (50 mL), brine (50 mL), dried over sodium sulfate and concentrated. The crude product was purified by preparative HPLC to obtain 2-(4-amino-3-(3,4-inden-1-difluoro-5-hydroxyphenyl)-1H-pyrazolo[3,4-d]pyrimidin-1-yl)-5-fluoro-2,3-dihydro-1H-one (12.4 mg) and 5-(4-amino-1-(5-fluoro-2,3-dihydro-1H-inden-2-difluorophenol (13.9 mg) as an off-white solid. Then, ethanolic HCl (5 mL) was added to both compounds and stirred for 30 min at RT. The reaction mixture was concentrated under reduced pressure and lyophilized to afford 2-(4-amino-3-(3,4-difluoro-5-hydroxyphenyl)-1H-pyrazolo[3,4-d]pyrimidin-1-yl)-5-fluoro-2,3-dihydro-1H-inden-1-one (12.5 mg) and 5-(4-amino-1-(5-fluoro-2,3-dihydro-1H-inden-2-yl)-1H-pyrazolo[3,4-d]pyrimidin-3-yl)-2,3-difluorophenol (14 mg) as the HCl salt (off-white solid). ¹HNMR (400 MHz, Methanol-d4): δ (ppm): 8.34 (s, 1H), 7.87 (dd, J=8.5, 5.3 Hz, 1H), 7.44-7.33 (m, 1H), 7.34-7.21 (m, 1H), 7.04 (d, J=7.1 Hz, 2H), 5.96 (dd, J=8.6, 5.3 Hz, 1H), 3.90 (dd, J=17.3, 8.6 Hz, 1H), 3.72 (dd, J=17.2, 5.3 Hz, 1H). Separation by chiral HPLC affords Compound Nos. 127a and 127b.

### Example 128: Preparation of Compound Nos. 128, 128a and 128b

### Synthesis of 5-[4-amino-1-(5-fluoro-2,3-dihydro-1H-inden-2-yl)-1H-pyrazolo[3,4-d]pyrimidin-3-yl]-2,3-difluorophenol

Same as provided in Example 127. ¹HNMR (400 MHz, Methanol-d4): δ (ppm): 8.41 (d, J=2.5 Hz, 1H), 7.25 (dd, J=8.3, 5.2 Hz, 1H), 7.07-6.97 (m, 2H), 6.94 (m, 2H), 5.87 (p, J=7.7 Hz, 1H), 3.65-3.43 (m, 4H). Separation by chiral HPLC affords Compound Nos. 128a and 128b.

### Example 129: Preparation of Compound No. 129

### Synthesis of 6-[4-amino-1-(2,3-dihydro-1H-inden-2-yl)-1H-pyrazolo[3,4-d]pyrimidin-3-yl]-8-fluoro-3,4-dihydro-2H-1,4-benzoxazin-3-one

### Step-1: [5-bromo-3-fluoro-2-(2-methoxy-2-oxo-ethoxy)phenyl]azinic acid

The compound (5-bromo-3-fluoro-2-hydroxy-phenyl)azinic acid (10 g, 42.3 mmol), K₂CO₃ (7.58 g, 55.0 mmol) and TBAI (15.6 g, 42.3 mmol) were added in 150 mL of CH₃CN followed by addition of methyl 2-chloroacetate (10.0 g, 93.06 mmol) to the mixture at 0° C. Then the reaction mixture was heated to 80° C. for 1 h. Then the solvent was evaporated under reduced pressure and the mixture was diluted with DCM (100 mL). The organic layer was washed water (2×50 mL) and the organic layer was further dried over Na₂SO₄, concentrated to get the desire product as yellow oil. Then, the compound was recrystallized from EtOH-water mixture to get the pure product as an off white solid.

### Step-2: 6-bromo-8-fluoro-4H-1,4-benzoxazin-3-one

[5-Bromo-3-fluoro-2-(2-methoxy-2-oxo-ethoxy)phenyl]azinic acid (10.69 g, 34.9 mmol), and iron powder (11.7 g, 209.4 mmol) were allowed to stir in 250 mL of AcOH overnight at R.T. Then the excess AcOH was neutralized by adding saturated NaHCO₃ soln (500 mL), followed by dilution with EtOAc (300 mL) and extracted. The organic layer was further washed with water (12×50 mL), brine and dried over Na₂SO₄. Then, the solvent was removed under reduced pressure to get a white solid product, used directly.

### Step-3: 8-fluoro-6-(4,4,5,5-tetramethyl-1,3,2-dioxaborolan-2-yl)-4H-1,4-benzoxazin-3-one

To a solution of 6-bromo-8-fluoro-4H-1,4-benzoxazin-3-one (1 g, 4.0 mmol) in 30 mL DMF, were added bis(pinacolato)diboron (1.55 g, 6.12 mmol) at RT. Then AcOK (1.2 g, 12.2 mmol) and the catalyst PdCl₂(dppf).DCM (160 mg, 0.2 mmol) were added and degassed with N₂ for 10-15 min. Then the mixture was heated at 90° C. for 3 h. The progress of the reaction was monitored by TLC and LCMS. After the completion of the reaction the mixture was allowed to cool at RT followed by addition of water (50 mL) and EtOAc (100 mL). The organic layer was extracted and further washed with water, brine, dried over Na₂SO₄ and concentrated to get the crude product. The crude product was further purified by silica gel column chromatography to obtain 8-fluoro-6-(4,4,5,5-tetramethyl-1,3,2-dioxaborolan-2-yl)-4H-1,4-benzoxazin-3-one (Yield: 830 mg).

### Step-4: 6-(4-amino-1-indan-2-yl-pyrazolo[3,4-d]pyrimidin-3-yl)-8-fluoro-4H-1,4-benzoxazin-3-one

8-Fluoro-6-(4,4,5,5-tetramethyl-1,3,2-dioxaborolan-2-yl)-4H-1,4-benzoxazin-3-one (390 mg, 1.39 mmol) was dissolved in 6 mL of DMF:water solution (1:1) followed by addition of 1-indan-2-yl-3-iodo-pyrazolo[3,4-d]pyrimidin-4-amine (373 mg, 0.99 mmol) to the solution. Then, Na₂CO₃ (263 mg, 2.47 mmol) and tetrakis(triphenyl phosphine)palladium (0) (114 mg, 0.09 mmol) were added to the mixture at RT. The reaction mixture was allowed to degas with N₂ for 10-15 min. Then, the mixture was allowed to heat at 90° C. for 3 h. The progress of the reaction was monitored by TLC and LCMS. After completion of reaction, the mixture was diluted with EtOAc (20 mL) and the mixture was extracted with water (12×5 mL), washed with brine and dried over dried over Na₂SO₄ and concentrated under reduced pressure to get the crude product which was purified by reverse phase HPLC to 6-(4-amino-1-indan-2-yl-pyrazolo[3,4-d]pyrimidin-3-yl)-8-fluoro-4H-1,4-benzoxazin-3-one (12 mg) as a white solid as the free base. Then the free base was dissolved in ethanolic HCl (5 mL) and stirred at RT for 15 min. The mixture was evaporated to dryness and further dried by lyophilization to get the corresponding HCl salt of 6-(4-amino-1-indan-2-yl-pyrazolo[3,4-d]pyrimidin-3-yl)-8-fluoro-4H-1,4-benzoxazin-3-one as a white powder (Yield: 14 mg). ¹HNMR (400 MHz, Methanol-d4) δ (ppm): 8.41 (s, 1H), 7.28-7.25 (m, 2H), 7.21 (m, 2H), 7.14 (d, J=10.7, 1.9 Hz, 1H), 7.01 (s, 1H), 5.87-5.81 (m, 1H), 4.73 (s, 2H), 3.63-3.51 (m, 4H). LCMS: 417 (M+1).

### Example 130: Preparation of Compound Nos. 130, 130a and 130b

### Synthesis of 5-[4-amino-1-(1,2,3,4-tetrahydronaphthalen-2-yl)-1H-pyrazolo[3,4-d]pyrimidin-3-yl]-2-fluoro-3-hydroxybenzonitrile

To a stirred solution of 3-iodo-1-(1,2,3,4-tetrahydronaphthalen-2-yl)-1H-pyrazolo[3,4-d]pyrimidin-4-amine (250 mg, 0.639 mmol) in DMF (5 mL) was added 3-cyano-4-fluoro-5-hydroxyphenylboronic acid (174 mg, 0.959 mmol) at RT. Then, Na₂CO₃ (203 mg, 1.91 mmol) dissolved in water (5 mL) was added to the reaction mixture followed by addition of Pd(PPh₃)₄ (51.7 mg, 0.044 mmol) at RT and the resultant reaction mixture was heated at 80° C. for 16 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude product was purified by preparative HPLC to obtain 5-(4-amino-1-(1,2,3,4-tetrahydronaphthalen-2-yl)-1H-pyrazolo[3,4-d]pyrimidin-3-yl)-2-fluoro-3-hydroxybenzonitrile (145 mg) as an off-white solid. To this was added ethanolic HCl (10 mL) and stirred for 30 min at RT. The reaction mixture was then concentrated under reduced pressure and lyophilized to afford 5-(4-amino-1-(1,2,3,4-tetrahydronaphthalen-2-yl)-1H-pyrazolo[3,4-d]pyrimidin-3-yl)-2-fluoro-3-hydroxybenzonitrile (147 mg) as the HCl salt (off-white solid). ¹HNMR (400 MHz, DMSO-d6) δ (ppm): 11.06 (s, 1H), 8.31 (s, 1H), 7.57-7.44 (m, 2H), 7.15 (q, J=4.9 Hz, 4H), 5.11 (s, 1H), 3.17 (m, 2H), 3.00 (m, 1H), 3.00 (s, 1H), 2.39-2.25 (m, 1H), 2.22 (m, 1H). Separation by chiral HPLC affords Compound Nos. 130a and 130b.

### Example 131: Preparation of Compound Nos. 131, 131a and 131b

### Synthesis of 5-[4-amino-1-(1,2,3,4-tetrahydronaphthalen-2-yl)-1H-pyrazolo[3,4-d]pyrimidin-3-yl]-2-fluoro-3-hydroxybenzamide

To a stirred solution of 5-(4-amino-1-tetralin-2-yl-pyrazolo[3,4-d]pyrimidin-3-yl)-2-fluoro-3-hydroxy-benzonitrile (50 mg, 0.0125 mmol) in t-butanol (6 mL) was added potassium hydroxide (35 mg, 0.625 mmol) at RT and the resultant reaction mixture was heated at 100° C. for 4 h. The reaction was monitored by LCMS. After 4 h, the reaction mixture was cooled to RT and concentrated under reduced pressure. The residue obtained diluted with EtOAc (70 mL). The organic layer was washed with water (2×20 mL), brine (50 mL), dried over sodium sulfate and concentrated. The crude product was purified by preparative HPLC to obtain 5-(4-amino-1-indan-2-yl-pyrazolo [3,4-d] pyrimidin-3-yl)-2-fluoro-3-hydroxy-benzamide (15 mg) as an off-white solid. Then, ethanolic HCl (5 mL) was added and the mixture stirred for 30 min at RT. The mixture was then concentrated under reduced pressure and lyophilized to afford 5-(4-amino-1-indan-2-yl-pyrazolo [3,4-d]pyrimidin-3-yl)-2-fluoro-3-hydroxy-benzamide (15.2 mg) as the HCl salt (off-white solid). ¹HNMR (400 MHz, Methanol-d4) δ (ppm): 8.38 (s, 1H), 7.50 (d, J=4.2 Hz, 1H), 7.41 (d, J=8.1 Hz, 1H), 7.14 (d, J=13.4 Hz, 4H), 5.25 (s, 1H), 3.60-3.49 (m, 1H), 3.21 (dd, J=16.0, 5.5 Hz, 1H), 3.09 (s, 2H), 2.56-2.44 (m, 1H), 2.31 (m, 1H). Separation by chiral HPLC affords Compound Nos. 131a and 131b.

### Example 132: Preparation of Compound No. 132

### Synthesis of 5-[4-amino-1-(2,3-dihydro-1H-inden-2-yl)-1H-pyrazolo[3,4-d]pyrimidin-3-yl]-2-fluoro-3-hydroxybenzamide

To a stirred solution of 5-(4-amino-1-indan-2-yl-pyrazolo[3,4-d]pyrimidin-3-yl)-2-fluoro-3-hydroxy-benzonitrile (60 mg, 0.155 mmol) in t-butanol (4 mL) was added potassium hydroxide (43.60 mg, 0.77 mmol) and the resultant reaction mixture was heated at 100° C. for 6 h. The reaction was monitored by TLC and by LCMS. After 6 h, the reaction mixture was cooled to RT and concentrated under reduced pressure. The residue obtained was diluted with EtOAc (80 mL). The organic layer was washed with water (22×5 mL), brine (30 mL), dried over sodium sulfate and concentrated. The crude product was purified by preparative HPLC to obtain 5-(4-amino-1-indan-2-yl-pyrazolo [3,4-d]pyrimidin-3-yl)-2-fluoro-3-hydroxy-benzamide (8 mg) as an off-white solid. Then, ethanolic HCl (5 mL) was added and the mixture stirred for 30 min at RT. The mixture was then concentrated under reduced pressure and lyophilized to afford 5-(4-amino-1-indan-2-yl-pyrazolo [3,4-d]pyrimidin-3-yl)-2-fluoro-3-hydroxy-benzamide (8.1 mg) as the HCl salt (off-white solid). ¹HNMR (400 MHz, Methanol-d4) δ (ppm): 8.41 (s, 1H), 7.47 (dd, J=5.6, 2.3 Hz, 1H), 7.37 (dd, J=7.9, 2.3 Hz, 1H), 7.30-7.17 (m, 4H), 5.86 (p, J=7.9 Hz, 1H), 3.57 (m, 4H).

### Example 133: Preparation of Compound No. 133

### Synthesis of 3-[3-(aminomethyl)phenyl]-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (300 mg, 0.795 mmol) in DMF (5 mL) was added [3-(aminomethyl) phenyl]boronic acid (223.6 mg, 1.19 mmol) at RT. Then, Na₂CO₃ (252.8 mg, 2.38 mmol) dissolved in water (5 mL) was added to the reaction mixture followed by addition of Pd(PPh₃)₄ (91.9 mg, 0.079 mmol) at RT and the resultant reaction mixture was heated at 100° C. for 2 h. The reaction was monitored by TLC and by LCMS. After completion of reaction, the reaction mixture was diluted with water (40 mL) and extracted with EtOAc (2×100 mL). The combined organic layers were washed with water (2×50 mL), brine (50 mL), dried over sodium sulfate and concentrated. The crude product was purified by preparative HPLC affording 63 mg 3-[3-(aminomethyl)phenyl]-1-indan-2-yl-pyrazolo[3,4-d]pyrimidin-4-amine as the formate salt (off white solid). ¹HNMR (400 MHz, Methanol-d4), 8.30 (s, 1H), 7.82-7.71 (m, 2H), 7.63 (t, J=7.6 Hz, 1H), 7.57 (d, J=7.7 Hz, 1H), 7.29-7.16 (m, 4H), 5.81 (p, J=8.2 Hz, 1H), 4.18 (s, 2H), 3.60 (dd, J=15.8, 7.8 Hz, 2H), 3.49 (dd, J=15.8, 8.5 Hz, 2H).

### Example 134: Preparation of Compound No. 134

### Synthesis of 5-[4-amino-6-chloro-7-(2,3-dihydro-1H-inden-2-yl)-7H-pyrrolo[2,3-d]pyrimidin-5-yl]-2,3-difluorophenol

### Step-1: Synthesis of 6-chloro-5-(3,4-difluoro-5-methoxyphenyl)-7-(2,3-dihydro-1H-inden-2-yl)-7H-pyrrolo[2,3-d]pyrimidin-4-amine

To a stirred solution of 5-(3,4-difluoro-5-methoxyphenyl)-7-(2,3-dihydro-1H-inden-2-yl)-7H-pyrrolo[2,3-d]pyrimidin-4-amine (30 mg, 0.076 mmol) in DCM (8 mL) was added N-chlorosuccinimide (20 mg, 0.15 mmol) and the mixture stirred at RT for 6 h. The progress of reaction was monitored by TLC and LCMS. The reaction mixture was subsequently portioned between water (10 mL) and DCM (3×50 mL). The combined organic extracts were washed with brine (10 mL), dried over anhydrous sodium sulfate and concentrated under reduced pressure to get 6-chloro-5-(3,4-difluoro-5-methoxyphenyl)-7-(2, 3-dihydro-1H-inden-2-yl)-7H-pyrrolo[2,3-d]pyrimidin-4-amine (60 mg).

### Step-2: Synthesis of 5-(4-amino-6-6-chloro-7-(2,3-dihydro-1H-inden-2-yl)-7H-pyrrolo[2,3-d]pyrimidin-5-yl)-2,3-difluoro phenol

To a stirred solution of 6-chloro-5-(3,4-difluoro-5-methoxyphenyl)-7-(2,3-dihydro-1H-inden-2-yl)-7H-pyrrolo[2,3-d]pyrimidin-4-amine (60 mg, 0.14 mmol) in DCM (5 mL) was added BBr₃ (1M in DCM; 0.6 mL, 0.56 mmol) at 0° C. dropwise, and the resultant reaction mixture was stirred at RT for 18 h. The reaction was monitored by TLC. After completion of reaction, the reaction mixture was slowly quenched using a saturated solution of NaHCO₃ (15 mL) and extracted with EtOAc (100 mL). The organic layer was washed with water (40 mL), brine (30 mL), dried over sodium sulfate and concentrated. The crude compound was purified by preparative HPLC to afford 9 mg of 5-(4-amino-6-6-chloro-7-(2, 3-dihydro-1H-inden-2-yl)-7H-pyrrolo[2, 3-d] pyrimidin-5-yl)-2,3-difluoro phenol as the free base. This product (8 mg) was dissolved in 2 M ethanolic-HCl (4 mL) and kept at RT for 20 min. The reaction mixture was then concentrated under reduced pressure and lyophilized to obtain 5-(4-amino-6-6-chloro-7-(2,3-dihydro-1H-inden-2-yl)-7H-pyrrolo[2,3-d]pyrimidin-5-yl)-2,3-difluoro phenol (8 mg) as an HCl salt (off-white solid). ¹H NMR (400 MHz, methanol-d4) δ (ppm): 8.29 (s, 1H), 7.27-7.21 (m, 4H), 6.89-6.86 (m, 2H), 5.90-5.83 (m, 1H), 3.86 (dd, J=15.8, 8.7 Hz, 2H), 3.47 (dd, J=15.9, 9.3 Hz, 2H).

### Example 135: Preparation of Compound No. 135

### Synthesis of 4-amino-5-(3,4-difluoro-5-hydroxyphenyl)-7-(2,3-dihydro-1H-inden-2-yl)-7H-pyrrolo[2,3-d]pyrimidine-6-carbonitrile

### Step-1: Synthesis of 6-bromo-5-(3,4-difluoro-5-methoxyphenyl)-7-(2, 3-dihydro-1H-inden-2-yl)-7H-pyrrolo[2,3-d]pyrimidin-4-amine

To a stirred solution of 5-(3,4-difluoro-5-methoxyphenyl)-7-(2,3-dihydro-1H-inden-2-yl)-7H-pyrrolo[2,3-d]pyrimidin-4-amine (100 mg, 0.25 mmol) in DCM (8 mL) was added N-bromo succinimide (45 mg, 0.25 mmol) and the reaction mixture stirred at RT for 1 h. The progress of reaction was monitored by TLC and LCMS. The reaction mixture was subsequently portioned between water (10 mL) and DCM (3×50 mL). The combined organic phases were extracts were washed with brine (10 mL), dried over anhydrous sodium sulfate and concentrated under reduced pressure to get 6-bromo-5-(3,4-difluoro-5-methoxyphenyl)-7-(2,3-dihydro-1H-inden-2-yl)-7H-pyrrolo[2,3-d]pyrimidin-4-amine (120 mg).

### Step-2: Synthesis of 4-amino-5-(3,4-difluoro-5-methoxyphenyl)-7-(2,3-dihydro-1H-inden-2-yl)-7H-pyrrolo[2,3-d]pyrimidine-6-carbonitrile

To a stirred solution of 6-bromo-5-(3,4-difluoro-5-methoxyphenyl)-7-(2,3-dihydro-1H-inden-2-yl)-7H-pyrrolo[2,3-d]pyrimidin-4-amine (120 mg, 0.25 mmol) and CuCN (69 mg, 0.76 mmol) in DMF (4 mL) was purged with nitrogen for 15 min. The reaction mixture was heated at 160° C. for 18 h. The progress of reaction was monitored by TLC and LCMS. The reaction mixture was subsequently portioned between ice water (10 mL) and DCM (3×50 mL). The combined organic extracts were washed with brine (10 mL), dried over anhydrous sodium sulfate and concentrated under reduced pressure to get 4-amino-5-(3, 4-difluoro-5-methoxyphenyl)-7-(2,3-dihydro-1H-inden-2-yl)-7H-pyrrolo[2,3-d]pyrimidine-6-carbonitrile (110 mg).

### Step-3: Synthesis of 4-amino-5-(3,4-difluoro-5-hydroxyphenyl)-7-(2,3-dihydro-1H-inden-2-yl)-7H-pyrrolo[2,3-d]pyrimidine-6-carbonitrile

To a stirred solution of 4-amino-5-(3,4-difluoro-5-methoxyphenyl)-7-(2,3-dihydro-1H-inden-2-yl)-7H-pyrrolo[2,3-d]pyrimidine-6-carbonitrile (100 mg, 0.239 mmol) in DCM (5 mL) was added BBr₃ (1M in DCM; 1.2 mL, 1.2 mmol) at 0° C. dropwise and the resultant reaction mixture was stirred at RT for 18 h. The reaction was monitored by TLC. After completion of reaction, the reaction mixture was slowly quenched using a saturated solution of NaHCO₃ (15 mL) and extracted with EtOAc (100 mL). The organic layer was washed with water (40 mL), brine (30 mL), dried over sodium sulfate and concentrated. The crude compound was purified by preparative HPLC to afford 9 mg of 4-amino-5-(3,4-difluoro-5-hydroxyphenyl)-7-(2,3-dihydro-1H-inden-2-yl)-7H-pyrrolo[2,3-d]pyrimidine-6-carbonitrile as a free base The product (8 mg) was dissolved in 2 M ethanolic-HCl (4 mL) and kept at RT for 20 min. The reaction mixture was then concentrated under reduced pressure and lyophilized to obtain 4-amino-5-(3,4-difluoro-5-hydroxyphenyl)-7-(2,3-dihydro-1H-inden-2-yl)-7H-pyrrolo[2,3-d]pyrimidine-6-carbonitrile (10 mg) as the HCl salt (off-white solid). ¹H NMR (400 MHz, methanol-d4) δ (ppm): 8.34 (s, 1H), 7.31-7.15 (m, 4H), 6.97 (dd, J=8.3, 6.0 Hz, 2H), 5.83 (m, 1H), 3.81 (dd, J=15.9, 9.1 Hz, 2H), 3.52 (dd, J=15.9, 9.2 Hz, 2H).

### Example 136: Preparation of Compound No. 136

### Synthesis of 5-[4-amino-7-(2,3-dihydro-1H-inden-2-yl)-6-fluoro-7H-pyrrolo[2,3-d]pyrimidin-5-yl]-2,3-difluorophenol

### Step-1: Synthesis of 5-(3,4-difluoro-5-methoxyphenyl)-7-(2, 3-dihydro-1H-inden-2-yl)-6-fluoro-7H-pyrrolo[2,3-d]pyrimidin-4-amine

A stirred solution of 5-(3,4-difluoro-5-methoxyphenyl)-7-(2,3-dihydro-1H-inden-2-yl)-7H-pyrrolo[2,3-d]pyrimidin-4-amine (600 mg, 1.53 mmol) in acetonitrile (30 mL) was cooled to 0° C. with an ice bath. Selectfluor (1.08 g, 3.06 mmol) was added to the reaction mixture and the reaction mixture was allowed to warm to room temperature and stirred for 15 min. The progress of reaction was monitored by TLC and LCMS. The reaction mixture showed presence of starting material as a major portion. The reaction mixture was subsequently portioned between water (50 mL) and DCM (3×100 mL). The combined organic extracts were washed with brine (50 mL), dried over anhydrous sodium sulfate and concentrated under reduced pressure to get 5-(3,4-difluoro-5-methoxyphenyl)-7-(2,3-dihydro-1H-inden-2-yl)-6-fluoro-7H-pyrrolo[2, 3-d]pyrimidin-4-amine (420 mg). (Conversion of product is 20% only by LCMS.)

### Step-2: Synthesis of 5-(4-amino-7-(2,3-dihydro-1H-inden-2-yl)-6-fluoro-7H-pyrrolo[2,3-d]pyrimidin-5-yl)-2,3-difluoro phenol

To a stirred solution of 5-(3,4-difluoro-5-methoxyphenyl)-7-(2,3-dihydro-1H-inden-2-yl)-6-fluoro-7H-pyrrolo[2,3-d]pyrimidin-4-amine (420 mg, 1.07 mmol) in DCM (25 mL) was added BBr₃ (1M in DCM; 5.4 mL, 5.36 mmol) at 0° C. dropwise and the resultant reaction mixture was stirred at RT for 18 h. The reaction was monitored by TLC. After completion of reaction, the reaction mixture was slowly quenched using a saturated solution of NaHCO₃ (50 mL) and extracted with EtOAc (3×100 mL). The organic layer was washed with water (40 mL), brine (50 mL), dried over sodium sulfate and concentrated. The crude product was purified by preparative HPLC to afford 40 mg of 5-(4-amino-7-(2, 3-dihydro-1H-inden-2-yl)-6-fluoro-7H-pyrrolo[2,3-d]pyrimidin-5-yl)-2,3-difluoro phenol as the free base. The product (8 mg) was dissolved in 2 M ethanolic-HCl (4 mL) and kept at RT for 20 min. The reaction mixture was then concentrated under reduced pressure and lyophilized to obtain 5-(4-amino-7-(2,3-dihydro-1H-inden-2-yl)-6-fluoro-7H-pyrrolo[2,3-d]pyrimidin-5-yl)-2,3-difluoro phenol (40 mg) as the HCl salt (off-white solid). ¹H NMR (400 MHz, Methanol-d4) δ (ppm): 8.36 (s, 1H), 7.28-7.21 (m, 4H), 6.89-6.85 (m, 2H), 5.76 (p, J=8.6 Hz, 1H), 3.70-3.61 (m, 2H), 3.52-3.49 (m, 2H).

### Example 137: Preparation of Compound No. 137

### Synthesis of 5-[4-amino-7-(2,3-dihydro-1H-inden-2-yl)-5H-pyrrolo[3,2-d]pyrimidin-5-yl]-2,3-difluorophenol

### Step 1: Synthesis of 5-(3,4-difluoro-5-methoxy-phenyl)-7-indan-2-yl-pyrrolo[3,2-d]pyrimidin-4-amine

7-Indan-2-yl-5H-pyrrolo[3,2-d]pyrimidin-4-amine (70 mg, 0.33 mmol), and 5-bromo-1,2-difluoro-3-methoxy-benzene were dissolved in 3 mL of DMSO followed by addition of K₂CO₃ (273 mg, 1.98 mmol) and L-proline (23 mg, 0.2 mmol). Then N₂ gas was purged through the reaction mixture for 5 min, and CuI (18 mg, 0.1 mmol) was added and N₂ gas was purged again for 5 more min. The mixture was heated at 90° C. overnight. The progress of the reaction was monitored by TLC and LCMS. Then the mixture was allowed to cool at RT followed by dilution with water (10 mL) and then the mixture was extracted with EtOAc (3×10 mL). The combined organic layer was washed with water (10 mL), brine (10 mL), dried over anhydrous sodium sulfate and concentrated under reduced pressure to get the crude product which was taken for the next reaction without any further purification.

### Step 2: Synthesis of 5-(4-amino-7-indan-2-yl-pyrrolo[3,2-d]pyrimidin-5-yl)-2,3-difluoro-phenol

5-(3,4-Difluoro-5-methoxy-phenyl)-7-indan-2-yl-pyrrolo[3,2-d]pyrimidin-4-amine (80 mg, 0.2 mmol) was dissolved in 1 M BBr₃ in DCM soln. (153 mg) at 0° C. and then the mixture was allowed to stir overnight at RT. The progress of the reaction was monitored by LCMS. After completion of reaction, the reaction mixture was added to ice-cold water and extracted with EtOAc (3×10 mL). The combined organic layer was washed with brine, dried over anhydrous sodium sulfate and concentrated under reduced pressure to get a crude product which was purified by reverse phase HPLC to get 5-(4-amino-7-indan-2-yl-pyrrolo[3,2-d]pyrimidin-5-yl)-2,3-difluoro-phenol (21 mg) as the formate salt (white solid), which was dried by lyophilization. ¹H NMR (400 MHz, methanol-d4) δ (ppm): 8.24 (s, 1H), 7.37 (s, 1H), 7.27-7.19 (m, 2H), 7.13 (dd, J=5.5, 3.2 Hz, 2H), 6.92-6.82 (m, 1H), 6.77 (d, J=6.6 Hz, 1H), 4.57 (s, 1H), 3.96 (p, J=8.0 Hz, 1H), 3.40 (dd, J=15.3, 7.8 Hz, 2H), 3.14 (dd, J=15.3, 8.2 Hz, 2H). LCMS: M+1=379.

### Example 138: Preparation of Compound No. 138

### Synthesis of 3-[4-(aminomethyl)phenyl]-1-(2,3-dihydro-1H-inden-2-yl)-1H-pyrazolo[3,4-d]pyrimidin-4-amine

To a solution of 1-(2,3-dihydro-1H-inden-2-yl)-3-iodo-1H-pyrazolo[3,4-d]pyrimidin-4-amine (200 mg, 0.53 mmol) in DMF (3 mL) was added [4-(aminomethyl) phenyl]boronic acid (148 mg, 0.79 mmol) at RT. Then, Na₂CO₃ (168 mg, 1.59 mmol) dissolved in water (3 mL) was added to the reaction mixture, followed by Pd(PPh₃)₄ (61 mg, 0.05 mmol) at RT, and the resultant reaction mixture was heated at 100° C. for 2 h. The reaction was monitored by TLC and LC-MS. After completion of reaction, the reaction mixture was diluted with water (20 mL) and extracted with EtOAc (2×75 mL). The combined organic layers were washed with water (2×50 mL), brine (50 mL), dried over sodium sulfate and concentrated. The crude product was purified by preparative HPLC affording 47 mg of 3-[4-(aminomethyl)phenyl]-1-indan-2-yl-pyrazolo[3,4-d]pyrimidin-4-amine as the free base (off-white solid). To this was added ethanolic-HCl (5 mL) and stirred for 30 min at RT. The reaction mixture was then concentrated under reduced pressure and lyophilized to afford 49.5 mg of 3-[4-(aminomethyl)phenyl]-1-indan-2-yl-pyrazolo[3,4-d]pyrimidin-4-amine as the HCl salt (off-white solid). ¹H NMR (400 MHz, methanol-d4) δ (ppm): 8.46 (s, 1H), 7.78 (d, J=8.0 Hz, 2H), 7.66 (d, J=8.0 Hz, 2H), 7.31-7.14 (m, 4H), 5.90 (m, 1H), 4.23 (s, 2H), 3.64-3.51 (m, 4H).

### Example 139: Preparation of Compound No. 139

### Synthesis of 5-[4-amino-1-(5-amino-2,3-dihydro-1H-inden-2-yl)-1H-pyrazolo[3,4-d]pyrimidin-3-yl]-2,3-difluorophenol

### Step-1: Synthesis of 5-nitroindan-2-ol

To a stirred solution of 5-nitroindan-2-one (500 mg, 2.824 mmol) in methanol (10 mL) was added sodium borohydride (215 mg, 5.648 mmol) at 0° C. portionwise. The reaction mixture was allowed to stir at 0° C. for 1 h. The reaction was monitored by TLC. After completion of reaction, the reaction mixture was concentrated under reduced pressure. The crude compound was diluted with water (200 mL) and extracted with EtOAc (2×250 mL). The combined organic layer was dried over sodium sulfate and concentrated to afford 5-nitroindan-2-ol (500 mg) which was taken to the next step without further purification.

### Step-2: Synthesis of (5-nitroindan-2-yl) methanesulfonate

To a stirred solution of 5-nitroindan-2-ol (500 mg, 3.067 mmol) in DCM (10 mL) was added triethylamine (1418 mg, 14.04 mmol) at 0° C. followed by the slow addition of methanesulfonyl chloride (640 mg, 5.616 mmol). The reaction was allowed to stir at 0° C. for 1 h. The reaction was monitored by NMR. After completion of reaction, the reaction mixture was diluted with water (150 mL) and extracted with DCM (3×200 mL). The combined organic layer was washed with brine (100 mL), dried over sodium sulfate and concentrated to afford (5-nitroindan-2-yl) methanesulfonate (700 mg) which was taken to the next step without further purification.

### Step-3: Synthesis of (5-aminoindan-2-yl) methanesulfonate

To a stirred solution of (5-nitroindan-2-yl) methanesulfonate (700 mg, 2.73 mmol) in methanol (8 mL) was added Fe powder (460 mg, 8.22 mmol) followed by addition of concentrated HCl (0.9 mL, 0.3 mL/mmol). The resulting reaction mixture was heated at 60° C. for 2.5 h. The reaction was monitored by TLC. After completion of reaction, the reaction mixture was concentrated under reduced pressure. The residue obtained was diluted with water (200 mL) and extracted with EtOAc (2×300 mL). The combined organic layer was washed with brine (100 mL), dried over sodium sulfate and concentrated. The crude compound obtained was triturated with acetone-hexane (1:5) to afford (5-aminoindan-2-yl) methanesulfonate (350 mg) which was taken to the next step without further purification.

### Step-4: Synthesis of 1-(5-aminoindan-2-yl)-3-iodo-3H-pyrazolo[3,4-d]pyrimidin-4-amine

To a stirred solution of 3-iodo-1H-pyrazolo[3,4-d]pyrimidin-4-amine (203 mg, 0.7812 mmol) in DMF was added cesium carbonate (507 mg, 1.5625 mmol) and the reaction mixture was allowed to stir at RT for 1 h. Then, TBAI (28 mg, 0.078 mmol) and (5-aminoindan-2-yl) methanesulfonate (350 mg, 1.5625 mmol) were successively added to the reaction mixture and the resultant reaction mixture was heated at 70° C. for 1.5 h. The reaction was monitored with TLC and LC-MS. After completion of reaction, the reaction mixture diluted with water (200 mL) and extracted with EtOAc (2×300 mL). The combined organic layer was washed with brine (100 mL), dried over sodium sulfate and concentrated. The crude compound obtained was triturated with acetone-hexane (1:5) to afford 1-(5-aminoindan-2-yl)-3-iodo-3H-pyrazolo[3,4-d]pyrimidin-4-amine (220 mg) as a light brown solid.

### Step-5: Synthesis of 5-[14-amino-1-(5-aminoindan-2-yl)-3H-pyrazolo[3,4-d]pyrimidin-3-yl]-2,3-difluoro-phenol

To a stirred solution of 1-(5-aminoindan-2-yl)-3-iodo-3H-pyrazolo[3,4-d]pyrimidin-4-amine (200 mg, 0.5102 mmol) in DMF (5 mL) was added 3,4-difluoro-5-hydroxyphenyl boronic acid (142 mg, 0.8116 mmol) and allowed to stirred for 2 min. Then, a solution of sodium carbonate (163 mg, 1.53 mmol) in water (5 mL) was added to the reaction mixture followed by addition of Pd(PPh₃)₄ (42 mg, 0.036 mmol). The resultant reaction mixture was heated 90° C. for 16 h. The reaction was monitored by TLC and LC-MS. After completion of reaction, the reaction mixture cooled to RT and diluted with water (200 mL). The aqueous layer was extracted with EtOAc (2×300 mL). The combined organic layer was washed with brine (100 mL), dried over sodium sulfate and concentrated. The crude compound obtained was purified by reverse phase preparative HPLC to obtain 5-[4-amino-1-[2-(3-pyridylmethyl amino)ethyl]pyrazolo[3,4-d]pyrimidin-3-yl]-2,3-difluoro-phenol (65 mg) as the free base. The product was dissolved in 2 M ethanolic HCl (4 ml) and kept at RT for 20 min. The reaction mixture was concentrated under reduced pressure to obtain 5-[4-amino-1-[2-(3-pyridylmethylamino)ethyl]pyrazolo[3,4-d]pyrimidin-3-yl]-2,3-difluoro-phenol as the HCl salt (61.65 mg). ¹H NMR (400 MHz, Methanol-d4) δ (ppm): 8.44 (s, 1H), 7.45 (d, J=8.0 Hz, 1H), 7.32 (s, 1H), 7.25 (d, J=7.9 Hz, 1H), 6.99 (t, J=8.1 Hz, 2H), 5.94 (p, J=7.3 Hz, 1H), 3.60 (dt, J=18.0, 8.9 Hz, 4H). LCMS: M+1=395.

### Example 140: Preparation of Compound No. 140

### Synthesis of 5-[8-amino-3-(2,3-dihydro-1H-inden-2-yl)imidazo[1,5-a]pyrazin-1-yl]-2,3-difluorophenol

To a solution of 3-(2,3-dihydro-1H-inden-2-yl)-1-iodoimidazo[1,5-a]pyrazin-8-amine (300 mg, 0.82 mmol) in DMF (5 mL) was added (3, 4-difluoro-5-hydroxy-phenyl) boronic acid (288 mg, 1.65 mmol) at RT. Then a solution of Na₂CO₃ (260 mg, 2.46 mmol) in water (5 mL) was added to the reaction mixture followed by addition of Pd(PPh₃)₄ (66.3 mg, 0.057 mmol) at RT, and the resultant reaction mixture was heated at 80° C. for 2 h. The reaction was monitored by TLC and LC-MS. After completion of reaction, the reaction mixture was diluted with water (40 mL) and extracted with EtOAc (2×100 mL). The combined organic layer was washed with water (2×50 mL), brine (50 mL) then dried over sodium sulfate and concentrated under reduced pressure. The crude compound was purified by preparative HPLC affording 5-(8-amino-3-(2,3-dihydro-1H-inden-2-yl)imidazole[1,5-a]pyrazin-1-yl)-2, 3-difluorophenol as the free base. To this was added ethanolic —HCl (10 mL) and stirred for 30 min at RT. The reaction mixture was then concentrated under reduced pressure and lyophilized to afford (163 mg) 5-(8-amino-3-(2,3-dihydro-1H-inden-2-yl)imidazole[1,5-a]pyrazin-1-yl)-2, 3-difluorophenol as the HCl salt (160 mg). ¹H NMR (400 MHz, methanol-d4) δ (ppm): 7.86 (d, J=5.9 Hz, 1H), 7.26 (dd, J=5.4, 3.4 Hz, 2H), 7.18 (dd, J=5.6, 3.2 Hz, 2H), 7.03 (t, J=7.3 Hz, 3H), 4.21 (p, J=8.6 Hz, 1H), 3.55-3.38 (m, 4H).

### Example P1: Preparation of Compound Nos. 2.1 to 2.278, and Stereoisomers Thereof

Compound Nos. 2.1 to 2.278, and stereoisomers thereof, can be prepared in an analogous fashion to the compounds described herein using appropriately functionalized starting materials and reagents known to those skilled in the art.

### Example B1: BTK and PI3Kδ Kinase Profiling of Compounds of the Invention

Compounds of the invention were profiled in a 10 dose IC₅₀ panel against BTK and PI3Kδ, both provided by Reaction Biology Corporation.

BTK assay: In vitro profiling protein kinases was performed using the “HotSpot” assay platform. Briefly, specific kinase/substrate pairs along with required cofactors were prepared in reaction buffer. Compounds were prepared as 10 mM stock solutions in DMSO. Compounds were delivered into the reaction, followed 15-20 minutes later by addition of a mixture of ATP (Sigma, St. Louis Mo.) and ³³P ATP (Perkin Elmer, Waltham Mass.) to a final concentration of 10 μM. Reactions were carried out at RT for 120 min, followed by spotting of the reactions onto P81 ion exchange filter paper (Whatman Inc., Piscataway, N.J.). Unbound phosphate was removed by extensive washing of filters in 0.75% Phosphoric acid. After subtraction of background derived from control reactions containing inactive enzyme, kinase activity data was expressed as the percent of remaining kinase activity in test samples compared to vehicle (DMSO) reactions. IC₅₀ values and curve fits were obtained using Prism (GraphPad Software). Both the percent inhibition values @ 1 and 0.1 μM, as well as IC₅₀ values are presented in Table B1. Buffer Conditions: 20 mM Hepes (pH 7.5), 10 mM MgCl₂, 1 mM EGTA, 0.02% Brij35, 0.02 mg/mL BSA, 0.1 mM Na₃VO₄, 2 mM DTT, 1% DMSO.

PI3Kδ assay: The PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI 3-Kinase(h) activity displaces biotin-PIP3 from the complex resulting in a loss of energy transfer and thus a decrease in signal. This is a 3-step reaction: First, the kinase reaction with PIP2 substrate is carried out in the presence of ATP (10 μM), and the reaction is quenched with stop Solution, and then finally detect by adding Detection Mixture followed by incubation (10 min). Reaction procedure: 1) Prepare substrate in freshly prepared Reaction Buffer. 2) Deliver kinase into the substrate solution and gently mix. 3) Deliver compounds in 100% DMSO (1 μM) into the kinase reaction mixture by Acoustic technology (Echo550; nanoliter range), incubate for 10 min at RT. 4) Deliver ATP into the reaction mixture to initiate the reaction. 5) Incubate for 30 min at 30° C. 6) Quench the reaction with Stop Solution. 7) Add Detection Mixture, and incubate for overnight. 8) Measure HTRF: Ex=320 nm, ratio of Em=615 nm and Em=665 nm. The emission ratio is converted into μM PIP3 production based on PIP3 standard curves. The nonlinear regression to obtain the standard curve and IC₅₀ values are performed using Graphpad Prism software. Both the percent inhibition values @ 1 and 0.1 μM, as well as IC₅₀ values are presented in Table B1. Assay Buffer: HEPES 50 mM (pH7.0), NaN3 0.02%, BSA 0.01%, Orthovanadate 0.1 mM, 1% DMSO. Detection buffer: HEPES 10 mM (pH7.0), BSA 0.02%, KF 0.16 M, EDTA 4 mM. Substrate: 10 μM PIP2 substrate (PI(4,5)P2).

TABLE B1
Kinase activity data - BTK & PI3Kδ kinases

| | BTK @ | BTK @ | BTK @ | BTK | PI3Kδ | PI3Kδ @ | PI3Kδ @ | PI3Kδ |
|---|---|---|---|---|---|---|---|---|
| Cpd | 1 μM | 0.1 μM | 0.01 μM | IC₅₀ | @ 1 μM | 0.1 μM | 0.01 μM | IC₅₀ |
| No. | (% Inh) | (% Inh) | (% Inh) | (μM) | (% Inh) | (% Inh) | (% Inh) | (μM) |
| 1 | | 12.61 | | | | 48.6 | | |
| 2 | | 78.45 | | 0.0124 | | 81.26 | | 0.0082 |
| 3 | | | | 0.0673 | | | | 0.0079 |
| 4 | | | | 0.584 | | | | 1.83 |
| 5 | 76.16 | 26.26 | | 1.97, 1.1 | 54.63 | 25.0 | | 2.79, 1.22 |
| 6 | | | | 0.0864 | | | | 0.0086 |
| 6a | | 63.12 | | 0.0811 | | 77.7 | | 0.0091 |
| 6b | | 45.78 | | 0.112 | | 79.6 | | 0.0077 |
| 7 | | | | 0.891 | | | | 1.34 |
| 8 | | 48.87 | | 0.0261 | | 55.11 | | 0.033 |
| 9 | | 15.85 | | | | 44.86 | | |
| 10 | | 58.79 | | | | 22.39 | | |
| 11 | | 22.06 | | | | 45.08 | | |
| 12 | | 31.62 | | | | 6.22 | | |
| 13 | | | | 0.0698 | | | | 2.39 |
| 14 | | | | 0.217 | | | | 0.0079 |
| 14a | | | | 0.784 | | | | 0.102 |
| 14b | | | | 1.59 | | | | 0.0254 |
| 15 | | | | 0.565 | | | | 0.067 |
| 16 | | 24.0 | | | | 51.94 | | |
| 17 | | 41.16 | | 0.164 | | 63.68 | | 0.0094 |
| 18 | | 38.26 | | 0.258 | | 18.95 | | 0.108 |
| 19a | | | | 1.21 | | | | 0.0148 |
| 19b | | | | 1.27 | | | | 0.0064 |
| 20 | | | | 2.02 | | | | 0.157 |
| 21 | | | | 0.192 | | | | 0.104 |
| 22 | | | | 0.293 | | | | 0.0396 |
| 23 | | 15.45 | | | | 23.11 | | |
| 24 | | 5.45 | | | | 10.3 | | |
| 25 | | 20.21 | | | | 27.39 | | |
| 26 | | | | 0.0106 | | | | 0.0661 |
| 26a | | 84.11 | | 0.0104 | | 39.35 | | 0.114 |
| 26b | | 82.1 | | 0.0209 | | 47.94 | | 0.0552 |
| 27 | | 13.54 | | | | 4.7 | | |
| 28 | | 14.0 | | | | 15.27 | | |
| 29 | | 90.90 | | 0.0070 | | 61.73 | | 0.0613 |
| 30 | | 35.16 | | 0.217 | | 64.19 | | 0.013 |
| 31 | | 68.24 | | 0.0174 | | 44.38 | | 0.0336 |
| 31a | | 93.84 | 67.39 | 0.0095 | | 68.13 | 33.04 | 0.0277 |
| 31b | | 93.12 | 64.36 | 0.0121 | | 78.27 | 44.15 | 0.0106 |
| 32 | | 13.50 | | 0.877 | | 11.3 | | 1.49 |
| 33 | | 7.04 | | | | 27.41 | | |
| 34 | | 23.12 | | | | 51.63 | | |
| 35 | | 29.9 | | | | 77.02 | | |
| 36 | | 43.28 | | | | 56.42 | | |
| 36a | 72.83 | 26.99 | | 0.19 | 76.31 | 34.17 | | 0.0010 |
| 36b | 76.93 | 33.82 | | 0.123 | 79.26 | 40.07 | | 0.155 |
| 37 | | 29.59 | | | | 15.52 | | |
| 38 | | 15.94 | | | | 7.33 | | |
| 39 | 87.24 | 45.81, 37.23 | | | 67.63 | 24.12, 30.2 | | |
| 40 | | 66.35 | | | | 41.21 | | |
| 41 | | 4.89 | | | | 80.15 | | |
| 42 | | 23.06 | | | | 70.85 | | |
| 43 | −0.8 | 3.54 | | | 11.07 | 11.31 | | |
| 44 | 94.77 | 74.18 | | 0.043 | 86.35 | 14.07 | | 0.172 |
| 45 | 87.47 | 49.32 | | | 44.54 | 12.84 | | |
| 46 | 96.3 | 85.71 | | 0.0212 | 67.34 | 30.55 | | 1.29 |
| 47 | 48.47 | 9.61 | | | 100.0 | 78.41 | | |
| 48 | 16.21 | 1.56 | | | 11.95 | 7.12 | | |
| 49 | 96.31 | 75.02 | | 0.0571 | 89.73 | 57.87 | | 0.119 |
| 50 | 66.77 | 21.32 | | 1.26 | 79.98 | 39.05 | | 0.362 |
| 51 | 82.76 | 41.05 | | 0.422 | 98.33 | 73.20 | | 0.0657 |
| 52 | 24.21 | 9.25 | | | 37.25 | 6.493 | | |
| 53 | 76.1 | 30.42 | | 0.575 | 81.32 | 30.15 | | 0.645 |
| 54 | 72.19 | 27.64 | | 0.84 | 72.03 | 22.41 | | 0.924 |
| 55 | 50.29 | 14.00 | | 2.09 | 48.96 | 15.60 | | 10.4 |
| 56 | 98.64 | 90.244 | | 0.0135 | 99.12 | 69.44 | | 0.0332 |
| 57 | 93.26 | 63.18 | | 0.134 | 100.0 | 96.89 | | 0.0080 |
| 58 | 43.79 | 18.0 | | | 44.88 | 33.15 | | |
| 59 | 19.76 | 14.08 | | | 5.79 | 17.93 | | |
| 60 | 30.02 | 16.91 | | | 31.35 | 10.65 | | |
| 61 | 45.41 | 16.63 | | | 71.04 | 32.36 | | |
| 62 | 91.61 | 57.13 | | | 43.03 | 17.28 | | |
| 63 | 98.56 | 92.01 | | 0.0042 | 92.56 | 81.43 | | 0.0104 |
| 64 | 5.76 | 2.74 | | | 4.07 | 0.0 | | |
| 65 | 7.69 | 4.02 | | | 11.18 | −4.81 | | |
| 66 | 95.07 | 69.93 | | 0.0253 | 53.69 | 17.08 | | 0.461 |
| 67 | 87.64 | 43.63 | | 0.142 | 90.92 | 84.5 | | 0.0041 |
| 68 | 89.54 | 54.54 | | 0.0843 | 87.4 | 57.97 | | 0.0321 |
| 69 | 61.7 | 19.61 | | 0.791 | 60.21 | 4.96 | | 0.328 |
| 70 | 36.56 | 12.11 | | | 56.41 | 9.29 | | |
| 71 | 71.58 | 24.51 | | 0.392 | 89.24 | 47.56 | | 0.0639 |
| 72 | 7.63 | 6.81 | | | 85.89 | 55.46 | | |
| 73 | 95.31 | 72.03 | | 0.0305 | 52.39 | 14.37 | | 0.325 |
| 74 | 33.05 | 10.82 | | | 65.15 | 10.99 | | |
| 75 | 29.0 | 10.92 | | | 15.57 | −0.46 | | |
| 76 | 6.5 | 5.47 | | | 13.55 | −13.69 | | |
| 77 | 76.64 | 24.85 | | | 16.5 | −1.66 | | |
| 78 | 54.62 | 18.18 | | | 43.86 | 4.96 | | |
| 79 | 9.02 | 7.61 | | | 12.1 | 6.45 | | |
| 80 | 68.21 | 45.74 | | | 29.99 | 5.1 | | |
| 81 | 94.81 | 73.83 | | 0.0269 | 91.62 | 87.14 | | 0.0035 |
| 82 | 30.18 | 11.64 | | | 82.95 | 41.76 | | |
| 83 | 18.4 | 10.79 | | | 13.54 | −5.17 | | |
| 84 | 90.69 | 51.83 | | 0.104 | 88.36 | 53.06 | | 0.0348 |
| 85 | | 43.43 | 12.89 | 0.154 | | 59.95 | 35.56 | 0.0347 |
| 86 | | 21.34 | 8.08 | | | 20.84 | 17.21 | |
| 87 | | 78.95 | 34.28 | | | 8.16 | 13.47 | |
| 88 | | 17.55 | 7.67 | | | 15.89 | 18.98 | |
| 89 | | 30.58 | 8.84 | | | 17.95 | 0.76 | |
| 90 | | 6.79 | 7.39 | | | 44.39 | 9.23 | |
| 91 | | 33.72 | 8 | | | 19.59 | −1.19 | |
| 92 | | 3.3 | 1.24 | | | 9.31 | 11.71 | |
| 93 | | 96.32 | 77.86 | 0.0008 | | 59.57 | 24.46 | 0.0212 |
| 94 | | 22.48 | 8.16 | | | 71.5 | 29.77 | |
| 95 | | 11.01 | 7 | | | 25.54 | 3.09 | |
| 96 | | 12.19 | 7.68 | | | 40.18 | 32.92 | |
| 97 | | 14.06 | 11.7 | | | 29.59 | 24.66 | |
| 98 | | 12.62 | 8.24 | | | 19.14 | 14.09 | |
| 99 | | 56.81 | 17.69 | 0.0796 | | 39.74 | 17.4 | 0.167 |
| 100 | | 52.22 | 17.5 | 0.078 | | 71.71 | 29.01 | 0.0447 |
| 101 | | 94.7 | 78.34 | 0.00247 | | 62.53 | 26.61 | 0.0621 |
| 102 | 9.91 | 0.67 | | >1.0 | 100.0 | 93.99 | | 0.0025 |
| 103 | 89.82 | 47.24 | | 0.162 | 94.52 | 72.71 | | 0.0457 |
| 104 | 94.25 | 72.07 | | 0.0549 | 82.84 | 38.16 | | 0.282 |
| 105 | 59.18 | 27.50 | | | 34.72 | 16.0 | | |
| 107 | | | | 0.0087 | | | | 0.0129 |
| 108 | | 6.6 | 1.76 | | | 21.98 | 9.28 | |
| 109 | | 63.35 | 14.85 | | | 26.17 | 2.46 | |
| 110 | | 91.79 | 55.15 | 0.0025 | | 76.2 | 40.85 | 0.0119 |
| 111 | | 17.47 | 6.49 | | | 91.28 | 64.38 | |
| 112 | | 5.57 | 3.45 | | | 23.62 | 13.7 | |
| 113 | | 38.11 | 8.12 | | | 39.52 | 20.77 | |
| 114 | | 86.29 | 41.16 | 0.0202 | | 84.46 | 41.19 | 0.0232 |
| 115 | | 50.43 | 12.09 | 0.0801 | | 93.12 | 82.64 | 0.0019 |
| 116 | | 47.8 | 10.59 | 0.0718 | | 70.68 | 6.65 | 0.0779 |
| 117 | | 67.32 | 21.31 | | | 91.69 | 75.86 | |
| 118 | | 65.15 | 19.01 | | | 95.72 | 79.16 | |
| 119 | | 70.28 | 21.71 | | | 91.77 | 70.35 | |
| 120 | | 1.41 | 93.67 | | | 91.68 | 77.84 | |
| 121 | | 6.55 | 90.34 | | | 25.95 | 1.09 | |
| 122 | | 53.15 | 12.72 | | | 25.32 | −3.68 | |
| 123 | | −0.78 | 1.82 | | | 88.87 | 59.11 | |
| 124 | | 34.18 | 4.47 | | | 52.39 | 8.34 | |
| 125 | | 80.31 | 39.05 | 0.0142 | | 62.27 | 22.51 | 0.0328 |
| 126 | | 8.18 | 8.16 | | | 2.3 | 4.71 | |
| 127 | | 21.87 | 9.24 | | | 51.48 | 16.23 | |
| 128 | | 63.94 | 19.98 | | | 91.22 | 56.4 | |
| 129 | | 18.99 | 6.57 | | | 19.42 | 8.3 | |
| 130 | | −0.64 | −1.03 | | | 90.11 | 53.08 | 0.000269 |
| 131 | | 0.17 | −2.45 | | | 53.68 | 13.11 | |
| 132 | | 5.3 | 0.44 | | | 56.84 | 13.73 | |
| 133 | | −0.94 | −0.81 | | | 1.17 | −1.05 | |
| 134 | | 74.7 | 17.28 | | | 56.02 | −6.1 | |
| 135 | | 88.59 | 51.3 | | | 84.33 | 51.74 | |
| 136 | | 93.64 | 72.51 | | | 80.15 | 38.49 | |
| 137 | | 96.31 | 87.65 | | | 45.54 | 6.28 | |
| 138 | | −6.05 | −7.89 | | | 5.54 | −2.71 | |
| 139 | | 95.07 | 80.96 | | | 90.95 | 65.26 | |
| 140 | | 96.04 | 83.98 | | | 79.18 | 31.54 | |

### Example B2: PI3Kδ Isoform Profiling of Compounds of the Invention

Compounds of the invention were profiled in a 10 dose IC₅₀ panel against PI3Kα, PI3Kβ, and PI3Kγ kinases, provided by Reaction Biology Corporation, applied to the PI3Kδ protocol provided in Example B1. Both the percent inhibition values @ 0.3 μM, as well as IC₅₀ values are presented in Table B2.

TABLE B2
Kinase Activity Data: Related PI3K isoforms: PI3Kα, PI3Kβ, and PI3Kγ.

| | PI3Kα | PI3Kα | PI3Kβ | PI3Kβ | PI3Kγ | PI3Kγ |
|---|---|---|---|---|---|---|
| Compound | @ 0.3 μM | IC₅₀ | @ 0.3 μM | IC₅₀ | @ 0.3 μM | IC₅₀ |
| No. | (% Inh) | (μM) | (% Inh) | (μM) | (% Inh) | (μM) |
| 2 | | 0.0173 | | 0.0553 | | 0.0137 |
| 3 | | 0.0201 | | 0.0908 | | 0.0535 |
| 6 | | 0.0201 | | 0.105 | | 0.122 |
| 6a | 85.6 | | 55.06 | | 38.58 | |
| 6b | 91.35 | | 69.23 | | 45.57 | |
| 8 | 76.76 | | 49.48 | | 60.19 | |
| 17 | 60.41 | | 50.15 | | 56.88 | |
| 18 | 33.14 | | 34.21 | | 19.7 | |
| 26 | 53.79 | | 37.87 | | 38.75 | |
| 26a | | 0.143 | | 1.12 | | 0.405 |
| 26b | | 0.161 | | 0.787 | | 0.455 |
| 29 | | 0.0731 | | 0.418 | | 0.196 |
| 31 | 50.6 | 0.539 | 52.63 | 0.927 | 0.74 | 6.46 |
| 31b | | 0.249 | 32.34 | | 14.76 | |
| 49 | | 0.504 | 19.09 | | 30.41 | |
| 56 | | 0.186 | 27.87 | | 62.17 | |
| 57 | | 0.0711 | 60.98 | | 30.85 | |
| 63 | | 0.0747 | 50.79 | | 33.44 | |
| 68 | | 0.014 | 20.68 | | 51.6 | |
| 81 | | 0.0211 | 73.05 | | 68.03 | |
| 84 | | 0.638 | 27.83 | | 55.61 | |
| 93 | | 0.0509 | 47.09 | | 72.89 | |
| 100 | | 0.0701 | 39.8 | | 57.52 | |
| 101 | | 0.12 | 41.93 | | 57.6 | |
| 102 | | 0.0211 | 67.67 | | 57.81 | |
| 107 | | 0.0565 | 49.12 | | 45.49 | |
| 110 | | 0.0835 | 50.12 | | 47.05 | |
| 115 | | 0.0132 | 68.12 | | 59.21 | |

### Example B3: BMX/ETK, ITK and TEC Kinase Profiling of Compounds of the Invention

Compounds of the invention were profiled in a 10 dose IC₅₀ panel against BMX/ETK, ITK and TEC kinases, provided by Reaction Biology Corporation, applied to the BTK protocol provided in Example B1. Both the percent inhibition values @ 0.3 μM, as well as IC₅₀ values are presented in Table B3.

TABLE B3
Kinase Activity Data: BMX/ETK, ITK and TEC kinases

| | | BMX/ | | | | |
|---|---|---|---|---|---|---|
| | BMX/ETK | ETK | ITK | ITK | TEC | TEC |
| Compound | @ 0.3 μM | IC₅₀ | @ 0.3 μM | IC₅₀ | @ 0.3 μM | IC₅₀ |
| No. | (% Inh) | (μM) | (% Inh) | (μM) | (% Inh) | (μM) |
| 2 | | 0.0077 | | >100.0 | | 0.1 |
| 3 | | 0.189 | | 46.9 | | 0.182 |
| 6 | | 0.184 | | 16.4 | | 0.28 |
| 6a | 84.79 | | 4.58 | | 41.28 | |
| 6b | 71.49 | | 0.04 | | 27.3 | |
| 8 | 69.01 | | 0.02 | | 20.18 | |
| 17 | 68.46 | | −0.36 | | 11.45 | |
| 18 | 73.26 | | −3.29 | | 10.01 | |
| 26 | 92.36 | | 2.15 | | 66.1 | |
| 26a | | 0.0031 | | >100.0 | | 0.0493 |
| 26b | | 0.0062 | | 51.6 | | 0.0821 |
| 29 | | 0.0008 | | >100.0 | | 0.0205 |
| 31 | 89.72 | 6.4 | 17.1 | >100.0 | 57.08 | 24.3 |
| 31b | 88.04 | | 6.31 | | 56.91 | |
| 49 | 75.85 | | 6.31 | | 44.68 | |
| 56 | 86.63 | | 8.82 | | 61.02 | |
| 57 | 74.27 | | 5.31 | | 40.21 | |
| 63 | 94.69 | | 3.68 | | 88.12 | |
| 68 | 78.92 | | −6.84 | | 42.69 | |
| 81 | 85.6 | | 2.1 | | 71.22 | |
| 84 | 62.51 | | −2.3 | | 23.9 | |
| 93 | 110.06 | | 108.85 | | 106.37 | |
| 100 | 107.37 | | 112.3 | | 102.76 | |
| 101 | 97.14 | | −2.77 | | 91.2 | |
| 102 | 4.6 | | −5.59 | | −3.89 | |
| 107 | 90.55 | | −5.41 | | 87.72 | |
| 110 | 92.59 | | −3.49 | | 89.37 | |
| 115 | 72.12 | | −9.43 | | 48.29 | |

### Example B4: B Cell Activation Assay

1 Female mouse approximately 2 months old was euthanized with CO₂ and the spleen removed. The tissue was homogenized with a polished glass microscope slide. The cells were collected in 5 mL complete medium (RPMI+10% FBS+pen/strep), placed in a 15 mL falcon tube and centrifuged at 500×g for 5 min. The supernatant was discarded and the pellet was resuspended in 3 mL of ACK lysis buffer (NH₄Cl 0.17M, KHCO₃ 10 mM, EDTA-Na₂ 0.1 mM, pH 7.2), incubated at 37° C. for 5 min. 2 mL complete medium was added and the mixture centrifuged at 500×g for 5 min. The supernatant was discarded and the pellet resuspended in 1 mL complete medium. The mixture was passed through a 40 μM cell strainer and collect in 50 mL falcon tube. Enough medium was added to have a total of 10 mL. The cells were counted and seeded in 24 or 48-well plate in 1 mL complete medium at 10⁶ cells/mL. The plate was placed in a 37° C. incubator for 15 min. The cells were treated for 30 min at 37° C. with 100 nM test compound dissolved in 100% DMSO. 3 μL IgD (Accurate chemical and scientific corp.) was added at a final concentration of 3 μg/mL and the mixture incubated at 37° C. for 4 h. The cells were collected in 1.5 mL eppendorf tubes, centrifuged at 500×g for 5 min and then resuspended in 100 μL of PBS+2% FBS. The following antibodies were added: α-B220 PE (pharmingen #553090), α-CD69 APC (pharmingen #60689), live/dead fixable aqua dead cell stain kit (life technologies #L34957) all at 1:300, and the mixture incubated for 15 min at 4° C. in the dark. To this was added 900 μL of PBS+2% FBS and the mixture centrifuged at 500×g for 5 min. The supernatant was removed and the pellet resuspended in 300 μL of PBS+2% FBS and mixed well. The mixture was transferred to a cytometer tube. The samples were run in the FACS Canto II cytometer and quantified by the number of CD69+B220+ cells as a percentage of the total number of B220+ alive cells. The results are displayed both as % inhibition and as IC₅₀ values, in Table B4.

TABLE B4

| | CD69 | CD69 IC₅₀ |
|---|---|---|
| Compound No. | @ 100 nM (% Inh) | (μM) |
| 2 | 22.52 ± 18.41 (n = 5) | |
| 3 | 77.66 ± 22.65 (n = 6) | 0.1834 ± 0.1715 (n = 3) |
| 6a | 64.71 ± 19.01 (n = 6) | 0.1073 ± 0.0262 (n = 3) |
| 6b | 68.73 ± 21.60 (n = 6) | 0.1183 ± 0.1043 (n = 3) |
| 14b | 24.20 ± 12.24 (n = 6) | |
| 15 | −0.75 ± 6.93 (n = 3) | |
| 17 | 33.56 ± 13.70 (n = 5) | |
| 18 | 13.22 ± 11.84 (n = 5) | |
| 19a | 24.62 ± 10.79 (n = 6) | |
| 19b | 36.67 ± 6.35 (n = 6) | |
| 26a | 16.07 ± 15.99 (n = 5) | |
| 26b | 11.62 ± 20.93 (n = 5) | |
| 29 | 17.76 ± 20.69 (n = 9) | |
| 31a | 15.47 ± 13.75 (n = 8) | |
| 31b | −13.21 ± 4.55 (n = 3) | |
| 36a | 1.63 ± 7.31 (n = 3) | |
| 42 | 22.71 ± 5.53 (n = 3) | |
| 49 | 16.42 ± 20.45 (n = 9) | |
| 56 | 20.57 ± 19.79 (n = 9) | |
| 57 | 64.26 ± 21.20 (n = 6) | 0.1174 ± 0.0836 (n = 4) |
| 63 | 21.07 ± 27.71 (n = 9) | |
| 67 | 65.11 ± 18.09 (n = 6) | 0.1086 ± 0.0570 (n = 3) |
| 68 | 44.83 ± 29.77 (n = 6) | |
| 81 | 88.08 ± 32.59 (n = 5) | 0.1063 ± 0.0508 (n = 4) |
| 84 | 47.61 ± 24.72 (n = 15) | 0.1535 |
| 85 | 24.82 ± 9.20 (n = 3) | |
| 93 | −19.68 ± 39.78 (n = 5) | |
| 100 | 27.39 ± 22.07 (n = 5) | |
| 101 | −12.35 ± 33.92 (n = 5) | |
| 102 | 81.06 ± 20.76 (n = 7) | 0.1914 ± 0.1196 (n = 4) |
| 103 | 25.06 ± 22.54 (n = 5) | |
| 104 | 28.78 ± 19.62 (n = 5) | |
| 105 | 24.25 ± 33.84 (n = 5) | |
| 107 | −0.20 ± 16.10 (n = 3) | |
| 108 | 11.99 ± 7.58 (n = 3) | |
| 109 | 10.19 ± 14.12 (n = 3) | |
| 110 | −11.51 ± 15.89 (n = 3) | |
| 111 | 57.39 ± 5.05 (n = 3) | 0.1185 ± 0.0111 (n = 3) |
| 112 | 13.63 ± 17.68 (n = 3) | |
| 113 | 11.61 ± 11.81 (n = 3) | |
| 114 | 10.19 ± 13.54 (n = 3) | |
| 115 | 44.51 ± 5.23 (n = 3) | |
| 116 | 20.48 ± 16.90 (n = 3) | |
| 117 | 46.59 ± 11.89 (n = 3) | |
| 118 | 44.32 ± 10.35 (n = 3) | |
| 119 | 47.52 ± 5.57 (n = 3) | |
| 120 | −99.28 ± 22.26 (n = 3) | |
| 121 | −82.03 ± 14.71 (n = 3) | |
| 122 | 2.58 ± 4.93 (n = 3) | |
| 123 | 29.22 ± 4.64 (n = 3) | |
| 124 | 14.44 ± 2.64 (n = 3) | |
| 125 | 2.68 ± 2.32 (n = 3) | |
| 126 | 12.35 ± 7.52 (n = 3) | |
| 128 | 49.29 ± 5.74 (n = 3) | |
| 129 | 12.5 ± 13.16 (n = 3) | |
| 130 | 67.53 ± 15.54 (n = 3) | 0.1045 ± 0.0007 (n = 3) |
| 131 | 15.73 ± 14.11 (n = 3) | |
| 132 | 27.67 ± 13.46 (n = 3) | |
| 133 | 6.8 ± 14.52 (n = 3) | |

### Example B5: Cell Anti-Proliferation Assay

Compounds of the invention were profiled in a B-lymphocyte anti-proliferation assay using SUDHL6 cells. On Day 0, SUDHL cells (7,500) were plated in RPMI media with FBS medium in a 96-well plate. On Day 1, Compounds of the invention were charged on top of the plated cells with a 1% DMSO final concentration. On Day 3, [³H] Thymidine was added at 100 nCi/well. On Day 4, the cells were harvested using a cell harvester on a GF/C filter mat and dried in a hot air oven. Cell readout was obtained using a scintillation counter, and the activity of the compound was determined as an IC₅₀ value. Ranges are presented in Table B5.

TABLE B5
Cell anti-proliferative data

| Compound No. | Cell Activity |
|---|---|
| 3 | + |
| 6 | + |

Key:
+ (1 μM to 10 μM),
++ (100 nM to 1 μM),
+++ (<100 nM)

### Example B6: Collagen Antibody-Induced Arthritis Model

The efficacy of compounds of the invention in autoimmune diseases such as arthritis can be assessed by preclinical in-vivo models including, for example, collagen antibody-induced arthritis, provided by MD Biosciences, Inc. Balb/c, DBA/1, or C57BI/6 strains of mice are used, with positive controls of Dexamethasone or Enbrel, over a period of 10-18 days. Readouts include body weight, clinical signs, arthritis score, hind paw thickness, histology, cytokine analysis, and gene expression.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of the formula (I): 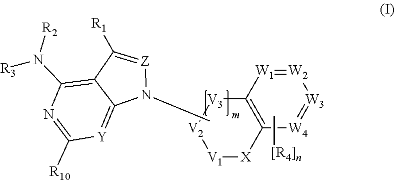
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ and —NHR₅;
V₁, V₂, and V₃, where present, are each independently: 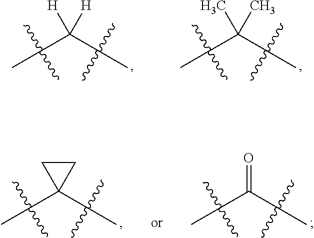
and one of V₁, V₂, or V₃ is 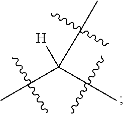 [SMILES: *C(*)*]
W₁, W₂, W₃ and W₄ are each independently CH or N, or CR₄ when R₄ is present;
X is CH₂ or NR₅;
Y is CH or N;
Z is N or CR₉;
m is 0 or 1;
n is 0 or an integer from 1 to 4;
R₅ is selected from the group consisting of H, —[C₁-C₆ alkyl]-CN, 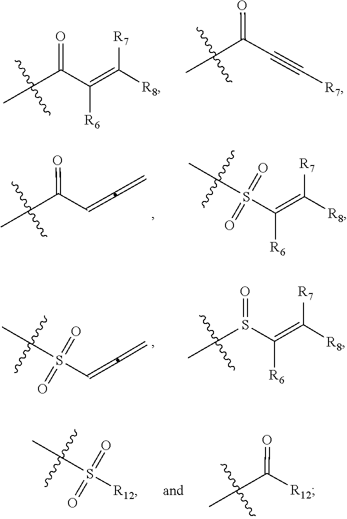
R₆ is H, or selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂ and —C(═O)NH₂;
R₇ and R₈ are each independently H, or selected from the group consisting of substituted or unsubstituted C₁-C₆ alkyl, substituted or unsubstituted cycloalkyl, substituted or unsubstituted heterocyclyl and substituted or unsubstituted amino;
R₉ is H, halo, CN, CF₃, —C(═O)NH₂, or substituted or unsubstituted C₁-C₆ alkyl;
R₁₀ is H, halo, —NHR₂ or substituted or unsubstituted C₁-C₆ alkyl; and
R₁₂ is substituted or unsubstituted C₁-C₆ alkyl or substituted or unsubstituted C₃-C₆ cycloalkyl.

2. A compound of the formula (II): 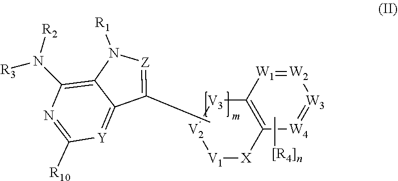
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino, and a substituted or unsubstituted thio;
R₂ and R₃ are each independently H or substituted or unsubstituted C₁-C₆ alkyl;
each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene and acyl;
V₁, V₂, and V₃, where present, are each independently selected from the group consisting of: 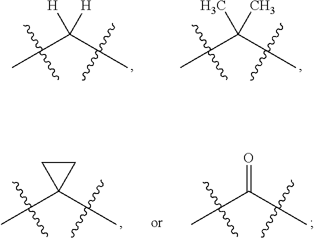
and one of V₁, V₂, or V₃ is 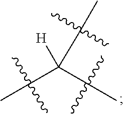 [SMILES: *C(*)*]
W₁, W₂, W₃ and W₄ are each independently CH or N, or CR₄ when R₄ is present;
X is CH₂ or NR₅;
Y is CH or N;
Z is N or CR₉;
m is 0 or 1;
n is 0 or an integer from 1 to 4;
R₅ is H, —[C₁-C₆ alkyl]-CN, 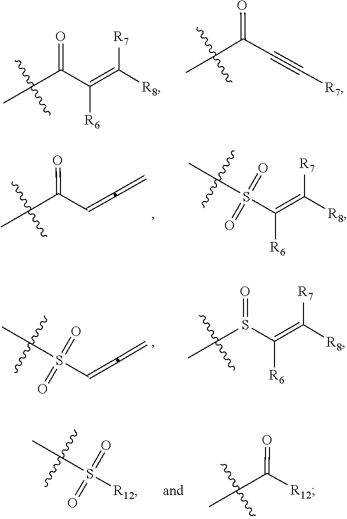
R₆ is H or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂ and —C(═O)NH₂;
R₇ and R₈ are each independently H, or is selected from the group consisting of substituted or unsubstituted C₁-C₆ alkyl, substituted or unsubstituted cycloalkyl, substituted or unsubstituted heterocyclyl and substituted or unsubstituted amino;
R₉ is H, halo, CN, CF₃, —C(═O)NH₂, or substituted or unsubstituted C₁-C₆ alkyl;
R₁₀ is H, halo, —NHR₂ or substituted or unsubstituted C₁-C₆ alkyl; and
R₁₂ is substituted or unsubstituted C₁-C₆ alkyl or substituted or unsubstituted C₃-C₆ cycloalkyl.

3. A compound of the formula (III): 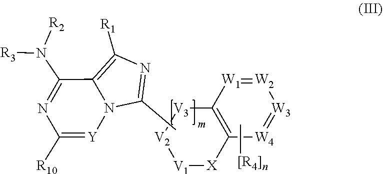
or a salt thereof;
wherein:
R₁ is selected from the group consisting of a substituted or unsubstituted C₁-C₆ alkyl, substituted or unsubstituted C₂-C₆ alkenyl, substituted or unsubstituted aryl, substituted or unsubstituted cycloalkyl, substituted or unsubstituted heterocyclyl, substituted or unsubstituted heteroaryl, substituted or unsubstituted alkoxy, substituted or unsubstituted aryloxy, substituted or unsubstituted amino and a substituted or unsubstituted thio;
R₂ and R₃ are each independently H or substituted or unsubstituted C₁-C₆ alkyl;
each R₄, where present, is independently selected from the group consisting of hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene and acyl;
V₁, V₂, and V₃, where present, are each independently selected from the group consisting of: 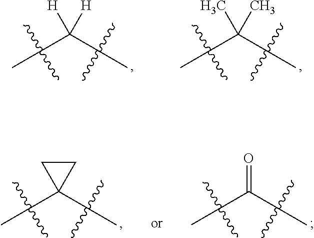
and one of V₁, V₂, or V₃ is 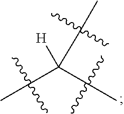 [SMILES: *C(*)*]
W₁, W₂, W₃ and W₄ are each CH or N, or CR₄ when R₄ is present;
X is CH₂ or NR₅;
Y is CH or N;
m is 0 or 1;
n is 0 or an integer from 1 to 4;
R₅ is H, —[C₁-C₆ alkyl]-CN, 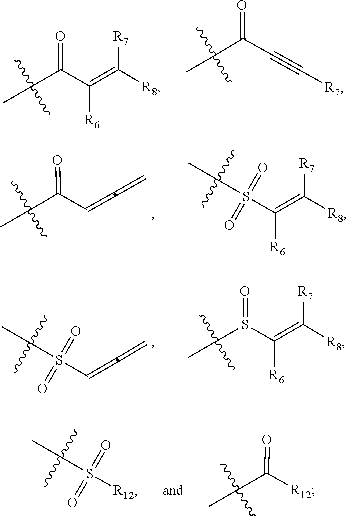
R₆ is H or is selected from the group consisting of substituted or unsubstituted C₁-C₆ alkyl, substituted or unsubstituted cycloalkyl, substituted or unsubstituted heterocyclyl, substituted or unsubstituted amino, CN, halo, —NO₂, CF₃, —SO₂CH₃, —SO₂NH₂, —C(═O)R₁₂, —C(═O)OR₁₂ and —C(═O)NH₂;
R₇ and R₈ are each independently H, or selected from the group consisting of substituted or unsubstituted C₁-C₆ alkyl, substituted or unsubstituted cycloalkyl, substituted or unsubstituted heterocyclyl and substituted or unsubstituted amino;
R₁₀ is H, halo, —NHR₂ or substituted or unsubstituted C₁-C₆ alkyl; and
R₁₂ is substituted or unsubstituted C₁-C₆ alkyl or substituted or unsubstituted C₃-C₆ cycloalkyl.

4. The compound of claim 1, wherein one of V₁, V₂, or V₃ where present, is bonded to the bicyclic heterocycle, and each of the remaining V₁, V₂, and V₃ where present, is —CH₂—.

5. The compound of claim 4, wherein one of the remaining V₁, V₂, and V₃ where present, is —C(CH₂—CH₂)—.

6. The compound of claim 1, wherein at least one of W₁, W₂, W₃ and W₄ is CR₄, where R₄ is selected from the group consisting of hydroxyl, nitro, cyano, halo, C₁-C₈ perhaloalkyl, C₁-C₈ alkyl, C₃-C₈ cycloalkyl, C₂-C₈ alkenyl, C₅-C₈ cycloalkenyl, C₂-C₈ alkynyl, aryl, heteroaryl, C₁-C₈ perhaloalkoxy, C₁-C₈ alkoxy, aryloxy, carboxyl, thiol, heterocyclyl, aralkyl, thioalkyl, amino, acylamino, aminoacyl, aminocarbonylamino, alkoxycarbonylamino, aminosulfonyl, sulfonylamino, sulfonyl, alkoxyalkylenecarbonyl, aminosulfonylalkylene and acyl.

7. The compound of claim 6, wherein one of W₁, W₂, W₃ and W₄ is N.

8. The compound of claim 1, wherein R₁ is selected from the group consisting of substituted or unsubstituted C₁-C₆ alkyl, substituted or unsubstituted aryl, substituted or unsubstituted heteroaryl, substituted or unsubstituted cycloalkyl and substituted or unsubstituted heterocyclyl.

9. The compound of claim 8, wherein R₁ is selected from the group consisting of phenyl or substituted phenyl, pyridyl or substituted pyridyl, pyrimidyl or substituted pyrimidyl, cyclohexyl or substituted cyclohexyl, indolyl or substituted indolyl, substituted or unsubstituted pyrrolopyridinyl and a substituted or unsubstituted indazolyl.

10. The compound of claim 9, wherein R₁ is unsubstituted phenyl or phenyl substituted with two or more substituents selected from the group consisting of hydroxyl, halo, perhaloalkyl, C₁-C₆ alkoxy, phenoxy, aminoacyl, substituted or unsubstituted amino, substituted or unsubstituted C₁-C₆ alkyl, cyano and allyloxy.

11. The compound of claim 9, wherein R₁ is a substituted or unsubstituted pyridin-2-yl, substituted or unsubstituted pyridin-3-yl, or substituted or unsubstituted pyridin-4-yl, wherein the substituent is selected from the group consisting of C₁-C₆alkoxy, substituted alkoxy, acyl, acyloxy, alkoxycarbonyl, acylamino, substituted or unsubstituted amino, aminoacyl, aminocarbonylamino, aminocarbonyloxy, aryl, substituted aryl, heteroaryl, substituted heteroaryl, aryloxy, substituted aryloxy, cyano, halo, hydroxyl, nitro, carboxyl, thiol, thioalkyl, substituted or unsubstituted C₁-C₆alkyl and substituted or unsubstituted cycloalkyl.

12. The compound of claim 9, wherein R₁ is selected from the group consisting of a substituted or unsubstituted benzimidazol-2-yl, substituted or unsubstituted benzimidazol-4-yl, substituted or unsubstituted benzimidazol-5-yl, a substituted or unsubstituted pyrazol-3-yl, or substituted or unsubstituted pyrazol-4-yl, a substituted or unsubstituted indol-1-yl, indol-2-yl, indol-3-yl, indol-4-yl, indol-5-yl, indol-6-yl, or indol-7-yl, a substituted or unsubstituted indol-1-yl, indol-2-yl, indol-6-yl, a substituted indol-1-yl, substituted indol-2-yl, substituted indol-6-yl, a substituted or unsubstituted pyrrolopyridin-2-yl, substituted or unsubstituted pyrrolopyridin-3-yl, substituted or unsubstituted pyrrolopyridin-4-yl, substituted or unsubstituted pyrrolopyridin-5-yl, substituted or unsubstituted pyrrolopyridin-6-yl, substituted or unsubstituted pyrrolopyridin-2-yl, substituted or unsubstituted pyrrolopyridin-5-yl, substituted pyrrolopyridin-5-yl, substituted or unsubstituted indazol-3-yl, substituted or unsubstituted indazol-4-yl, substituted or unsubstituted indazol-5-yl, substituted or unsubstituted indazol-6-yl and substituted or unsubstituted indazol-7-yl.

13. The compound of claim 1, wherein R₂ and R₃ are each H, or R₂ is H and R₃ is C₁-C₆ alkyl.

14. The compound of claim 1, wherein Y is CH or N.

15. The compound of claim 1, wherein Z is CR₉.

16. The compound of claim 1, wherein X is NR₅, wherein R₅ is selected from the group consisting of

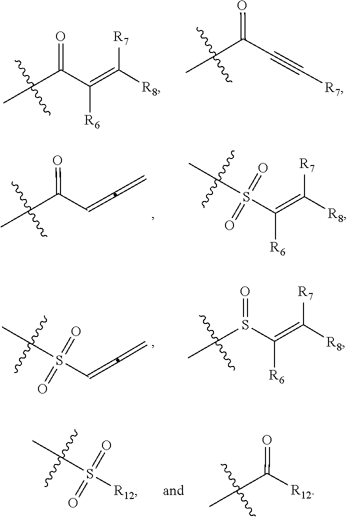

17. The compound of claim 16, wherein R₆ is H or is selected from the group consisting of CN, halo, —NO₂, CF₃, —SO₂CH₃, —SO₂NH₂, —C(═O)R₁₂, —C(═O)OR₁₂ and —C(═O)NH₂.

18. The compound of claim 1, wherein X is NR₅, m is 0, and the indoline moiety is attached to the heterocycle as an indolin-2-yl, indolin-3-yl, dihydroquinolin-2-yl, dihydroquinoline, dihydroquinolin-3-yl group, dihydroquinolin-4-yl group, dihydroinden-1-yl and dihydroinden-2-yl.

19. The compound of claim 1, wherein a tetrahydronaphthalene moiety is attached to the heterocycle as a tetrahydronaphthalen-1-yl or a tetrahydronaphthalen-2-yl group.

20. The compound of claim 1, wherein n is 1 or 2, and each R₄ is independently selected from the group consisting of hydroxyl, halo, C₁-C₈ perhaloalkyl, substituted or unsubstituted C₁-C₈ alkyl, acyl, substituted or unsubstituted amino, acylamino and aminoacyl.

21. The compound of claim 1, wherein the compound is of structure A-1, A-2, A-3, A-4, A-5, A-6, A-7, A-8, A-9, or A-10: 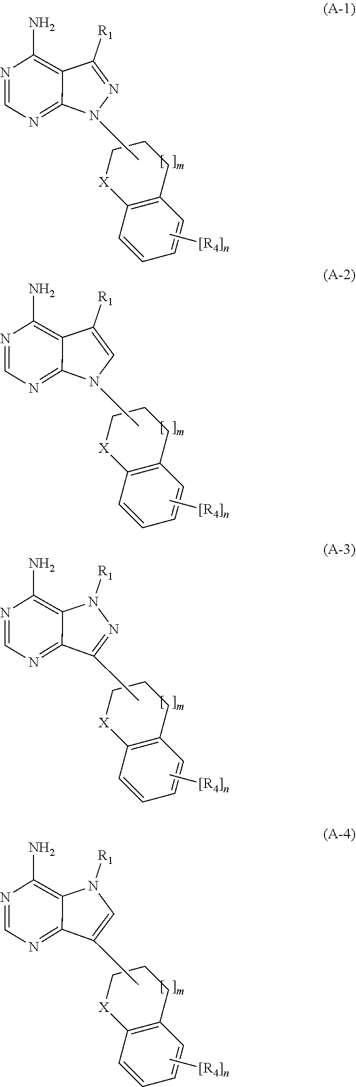 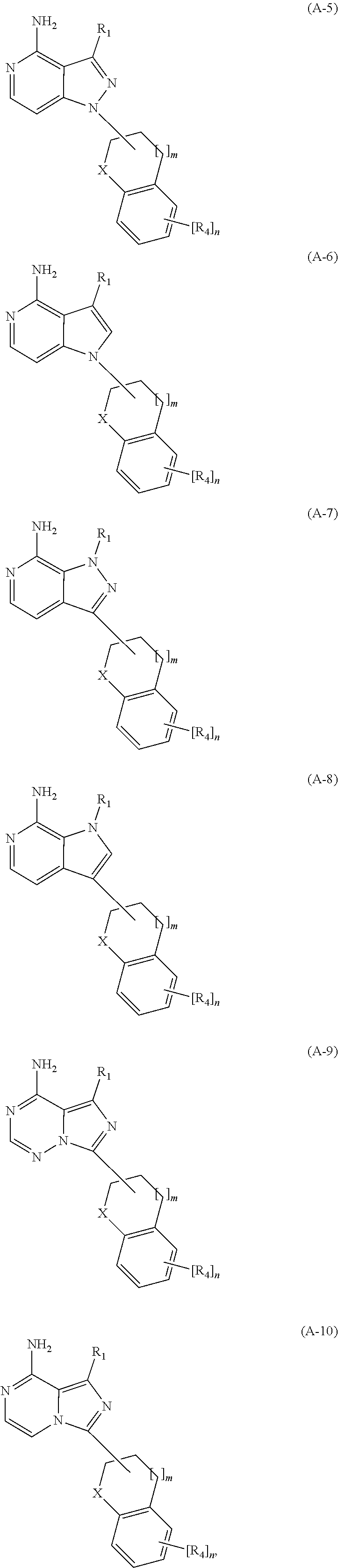
or a salt thereof; wherein R₁, X, m, n, R₄, are as described for formulae (I), (II) and (III).

22. The compound of claim 21, wherein the compound is of structure A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, or A-10e: 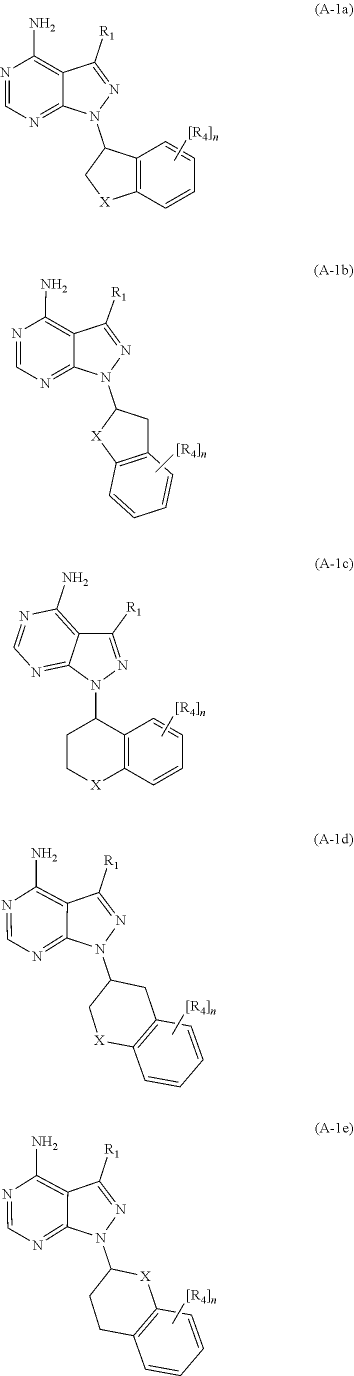 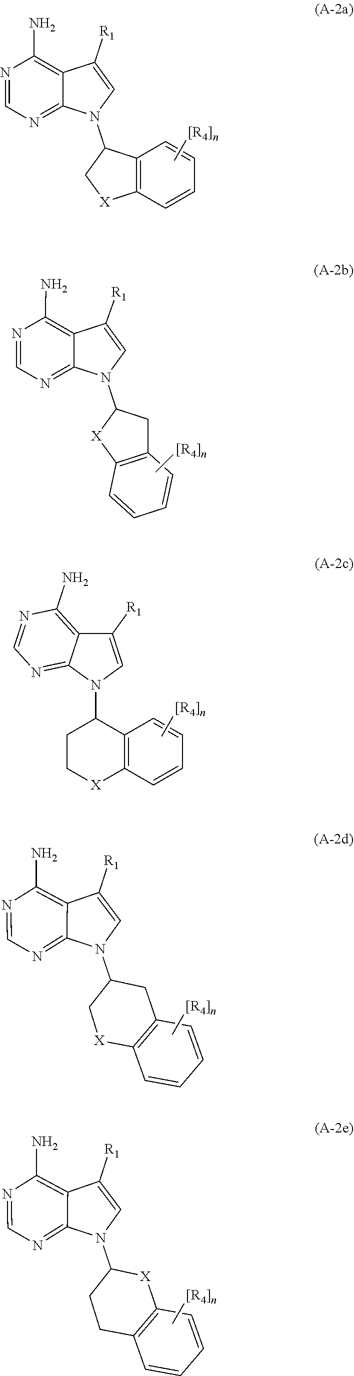 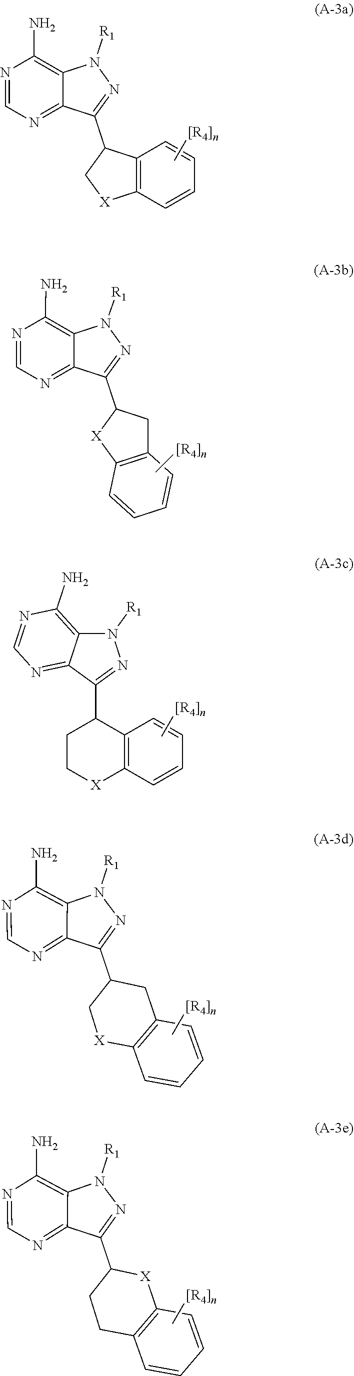 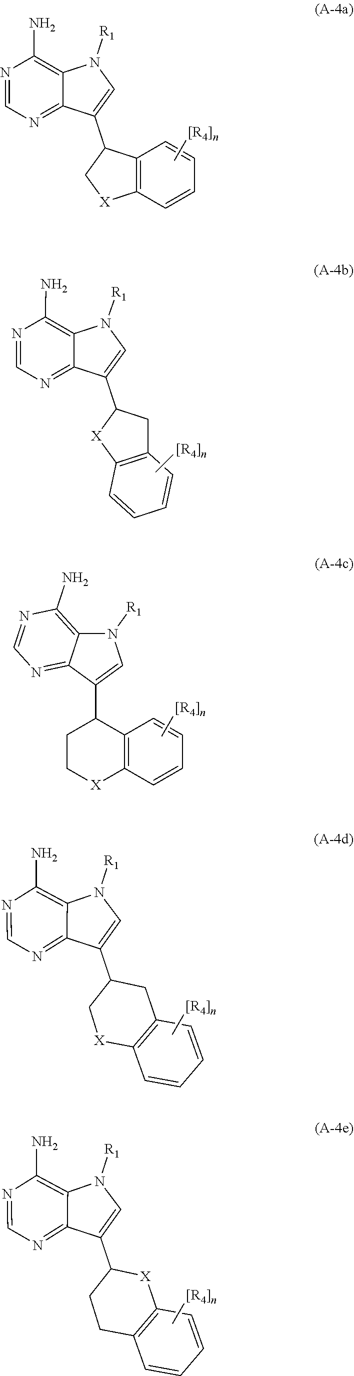 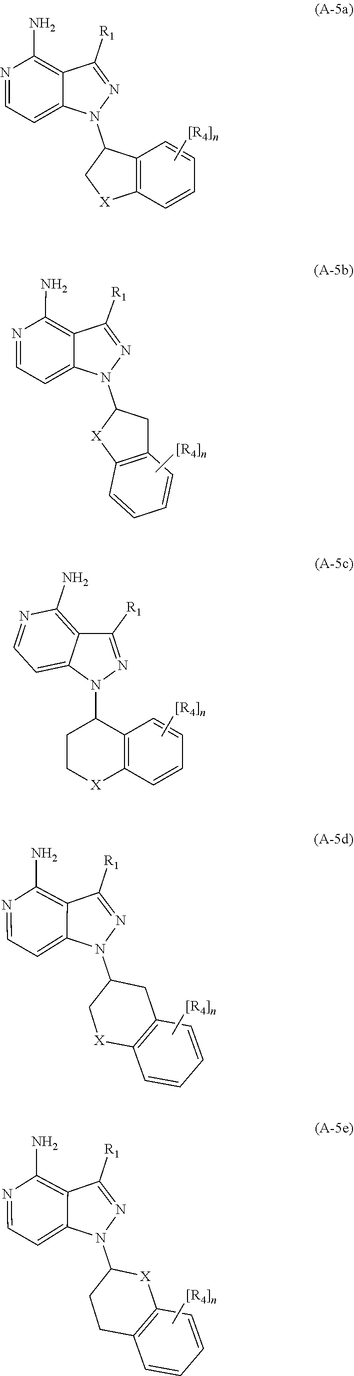 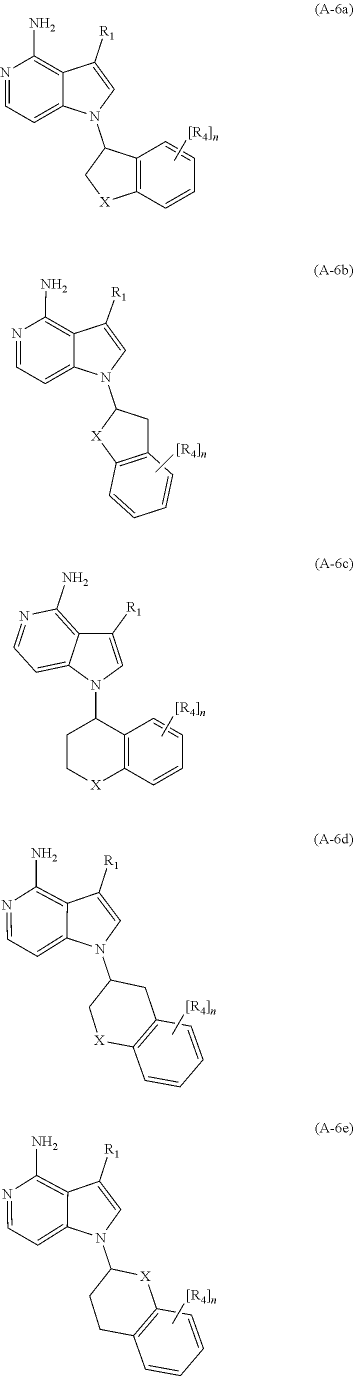 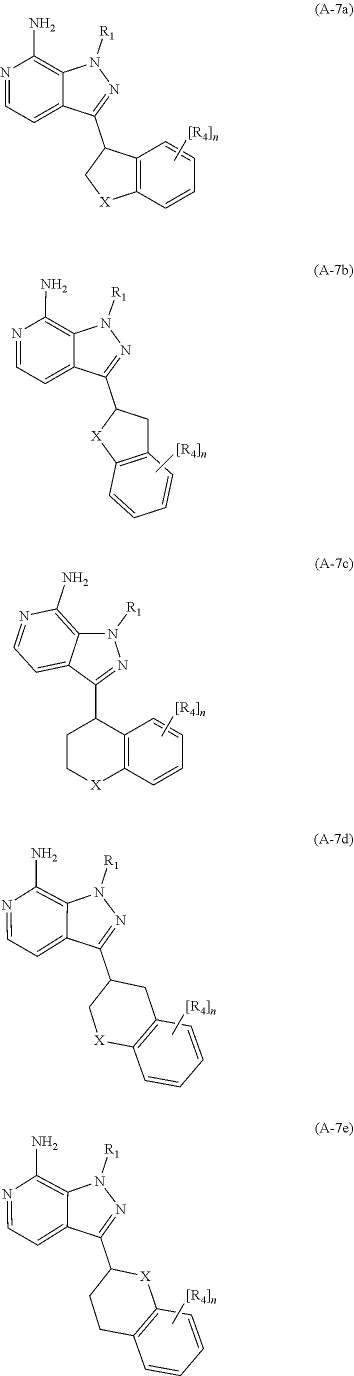 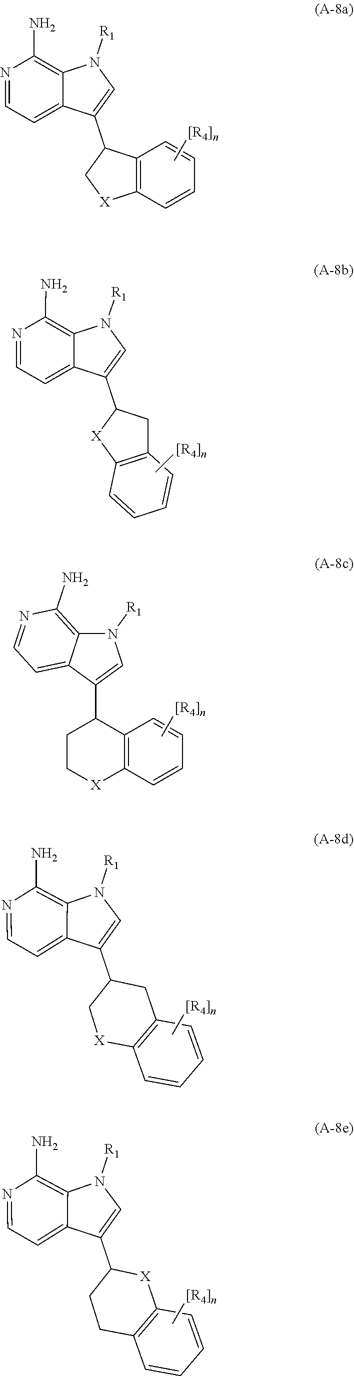 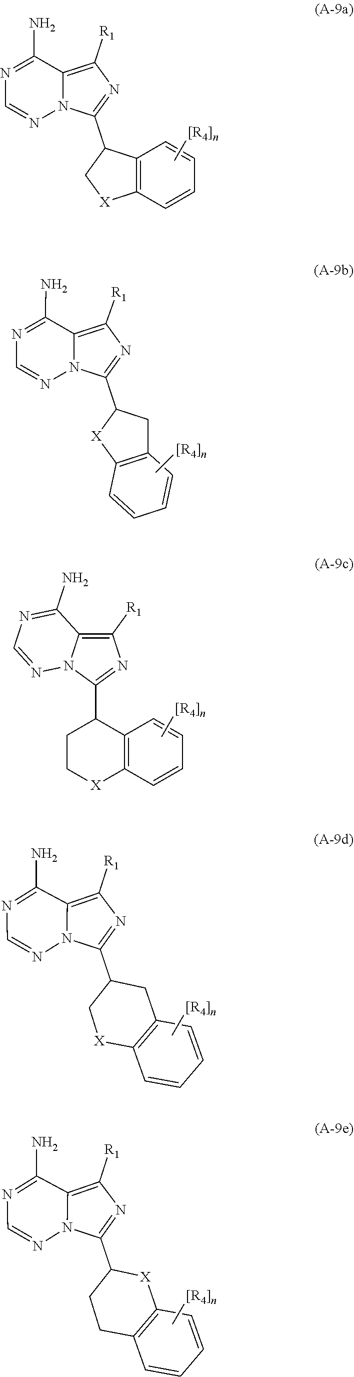 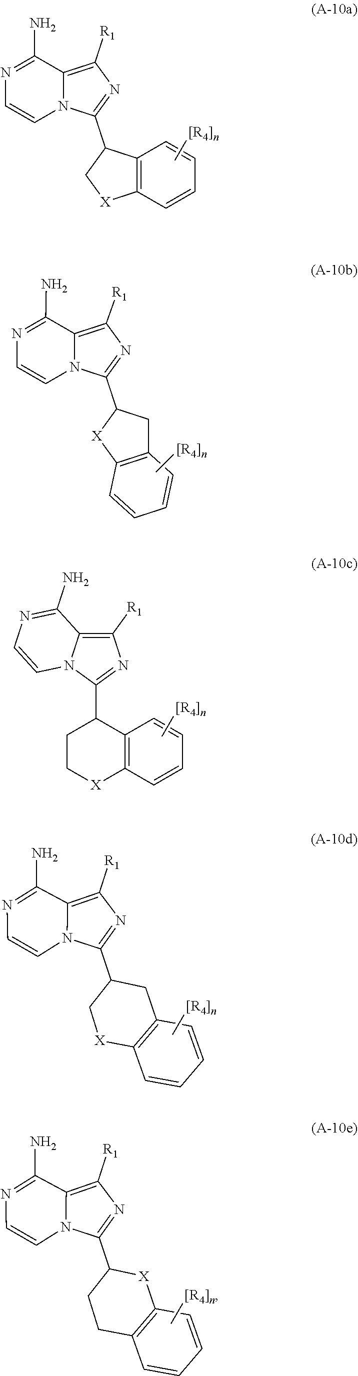
or a salt thereof; wherein R₁, R₄, X, n, R₅, R₆, R₇ and R₈, where present, are as described for formulae A-1, A-2, A-3, A-4, A-5, A-6, A-7, A-8, A-9 and A-10.

23. The compound of claim 22, wherein the compound is of structure A-1a-1, A-1a-2, A-1a-3, A-1a-4, A-1a-5, A-1a-6, A-1a-7, A-1a-8, A-1b-1, A-1b-2, A-1b-3, A-1b-4, A-1b-5, A-1b-6, A-1b-7, A-1b-8, A-1c-1, A-1c-2, A-1c-3, A-1c-4, A-1c-5, A-1c-6, A-1c-7, A-1c-8, A-1d-1, A-1d-2, A-1d-3, A-1d-4, A-1d-5, A-1d-6, A-1d-7, A-1d-8, A-1e-1, A-1e-2, A-1e-3, A-1e-4, A-1e-5, A-1e-6, A-1e-7, A-1e-8, A-2a-1, A-2a-2, A-2a-3, A-2a-4, A-2a-5, A-2a-6, A-2a-7, A-2a-8, A-2b-1, A-2b-2, A-2b-3, A-2b-4, A-2b-5, A-2b-6, A-2b-7, A-2b-8, A-2c-1, A-2c-2, A-2c-3, A-2c-4, A-2c-5, A-2c-6, A-2c-7, A-2c-8, A-2d-1, A-2d-2, A-2d-3, A-2d-4, A-2d-5, A-2d-6, A-2d-7, A-2d-8, A-2e-1, A-2e-2, A-2e-3, A-2e-4, A-2e-5, A-2e-6, A-2e-7, A-2e-8, A-3a-1, A-3a-2, A-3a-3, A-3a-4, A-3a-5, A-3a-6, A-3a-7, A-3a-8, A-3b-1, A-3b-2, A-3b-3, A-3b-4, A-3b-5, A-3b-6, A-3b-7, A-3b-8, A-3c-1, A-3c-2, A-3c-3, A-3c-4, A-3c-5, A-3c-6, A-3c-7, A-3c-8, A-3d-1, A-3d-2, A-3d-3, A-3d-4, A-3d-5, A-3d-6, A-3d-7, A-3d-8, A-3e-1, A-3e-2, A-3e-3, A-3e-4, A-3e-5, A-3e-6, A-3e-7, A-3e-8, A-4a-1, A-4a-2, A-4a-3, A-4a-4, A-4a-5, A-4a-6, A-4a-7, A-4a-8, A-4b-1, A-4b-2, A-4b-3, A-4b-4, A-4b-5, A-4b-6, A-4b-7, A-4b-8, A-4c-1, A-4c-2, A-4c-3, A-4c-4, A-4c-5, A-4c-6, A-4c-7, A-4c-8, A-4d-1, A-4d-2, A-4d-3, A-4d-4, A-4d-5, A-4d-6, A-4d-7, A-4d-8, A-4e-1, A-4e-2, A-4e-3, A-4e-4, A-4e-5, A-4e-6, A-4e-7, A-4e-8, A-5a-1, A-5a-2, A-5a-3, A-5a-4, A-5a-5, A-5a-6, A-5a-7, A-5a-8, A-5b-1, A-5b-2, A-5b-3, A-5b-4, A-5b-5, A-5b-6, A-5b-7, A-5b-8, A-5c-1, A-5c-2, A-5c-3, A-5c-4, A-5c-5, A-5c-6, A-5c-7, A-5c-8, A-5d-1, A-5d-2, A-5d-3, A-5d-4, A-5d-5, A-5d-6, A-5d-7, A-5d-8, A-5e-1, A-5e-2, A-5e-3, A-5e-4, A-5e-5, A-5e-6, A-5e-7, A-5e-8, A-6a-1, A-6a-2, A-6a-3, A-6a-4, A-6a-5, A-6a-6, A-6a-7, A-6a-8, A-6b-1, A-6b-2, A-6b-3, A-6b-4, A-6b-5, A-6b-6, A-6b-7, A-6b-8, A-6c-1, A-6c-2, A-6c-3, A-6c-4, A-6c-5, A-6c-6, A-6c-7, A-6c-8, A-6d-1, A-6d-2, A-6d-3, A-6d-4, A-6d-5, A-6d-6, A-6d-7, A-6d-8, A-6e-1, A-6e-2, A-6e-3, A-6e-4, A-6e-5, A-6e-6, A-6e-7, A-6e-8, A-7a-1, A-7a-2, A-7a-3, A-7a-4, A-7a-5, A-7a-6, A-7a-7, A-7a-8, A-7b-1, A-7b-2, A-7b-3, A-7b-4, A-7b-5, A-7b-6, A-7b-7, A-7b-8, A-7c-1, A-7c-2, A-7c-3, A-7c-4, A-7c-5, A-7c-6, A-7c-7, A-7c-8, A-7d-1, A-7d-2, A-7d-3, A-7d-4, A-7d-5, A-7d-6, A-7d-7, A-7d-8, A-7e-1, A-7e-2, A-7e-3, A-7e-4, A-7e-5, A-7e-6, A-7e-7, A-7e-8, A-8a-1, A-8a-2, A-8a-3, A-8a-4, A-8a-5, A-8a-6, A-8a-7, A-8a-8, A-8b-1, A-8b-2, A-8b-3, A-8b-4, A-8b-5, A-8b-6, A-8b-7, A-8b-8, A-8c-1, A-8c-2, A-8c-3, A-8c-4, A-8c-5, A-8c-6, A-8c-7, A-8c-8, A-8d-1, A-8d-2, A-8d-3, A-8d-4, A-8d-5, A-8d-6, A-8d-7, A-8d-8, A-8e-1, A-8e-2, A-8e-3, A-8e-4, A-8e-5, A-8e-6, A-8e-7, A-8e-8, A-9a-1, A-9a-2, A-9a-3, A-9a-4, A-9a-5, A-9a-6, A-9a-7, A-9a-9, A-9b-1, A-9b-2, A-9b-3, A-9b-4, A-9b-5, A-9b-6, A-9b-7, A-9b-8, A-9c-1, A-9c-2, A-9c-3, A-9c-4, A-9c-5, A-9c-6, A-9c-7, A-9c-8, A-9d-1, A-9d-2, A-9d-3, A-9d-4, A-9d-5, A-9d-6, A-9d-7, A-9d-8, A-9e-1, A-9e-2, A-9e-3, A-9e-4, A-9e-5, A-9e-6, A-9e-7, A-9e-8, A-10a-1, A-10a-2, A-10a-3, A-10a-4, A-10a-5, A-10a-6, A-10a-7, A-10A-10, A-10b-1, A-10b-2, A-10b-3, A-10b-4, A-10b-5, A-10b-6, A-10b-7, A-10b-8, A-10c-1, A-10c-2, A-10c-3, A-10c-4, A-10c-5, A-10c-6, A-10c-7, A-10c-8, A-10d-1, A-10d-2, A-10d-3, A-10d-4, A-10d-5, A-10d-6, A-10d-7, A-10d-8, A-10e-1, A-10e-2, A-10e-3, A-10e-4, A-10e-5, A-10e-6, A-10e-7, or A-10e-8: 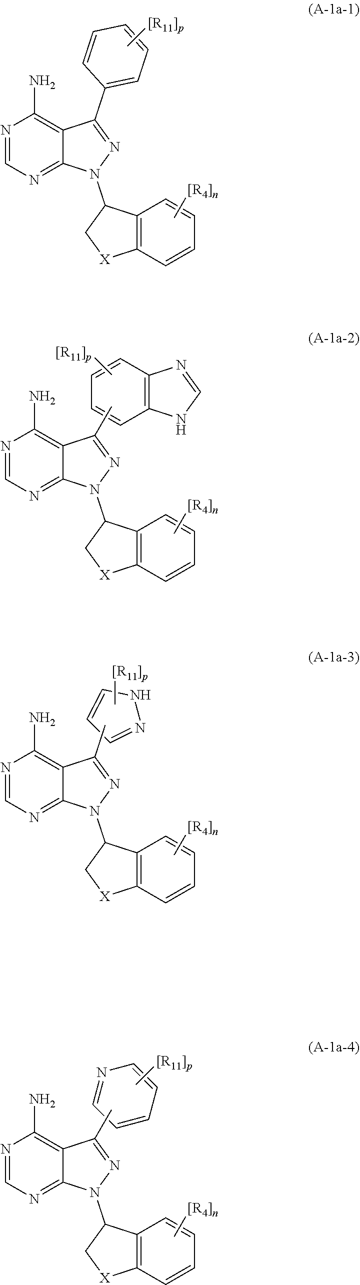 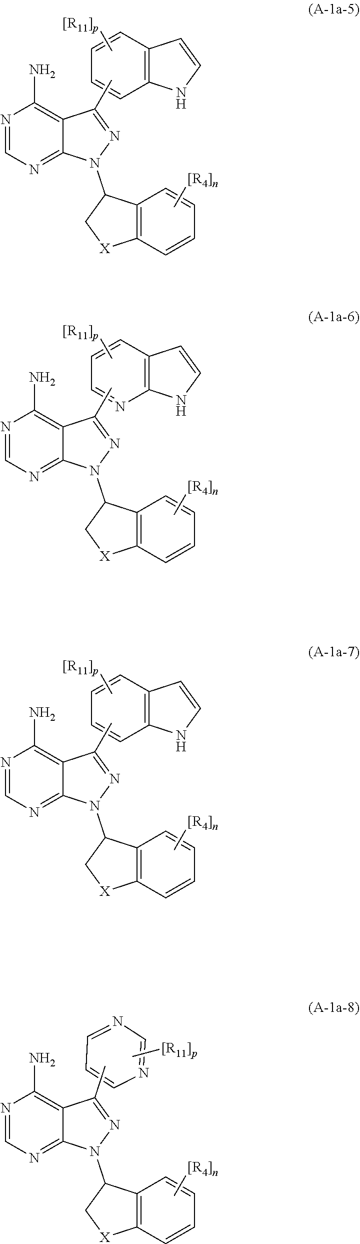 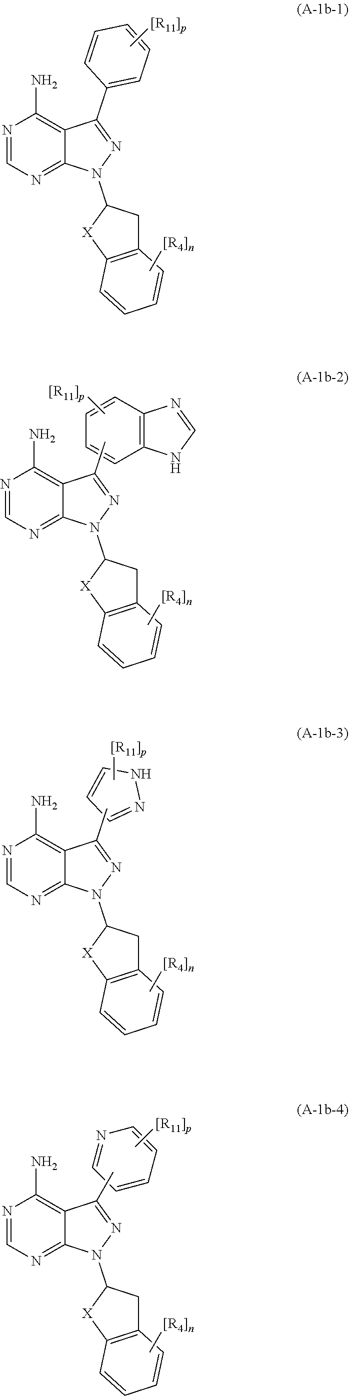 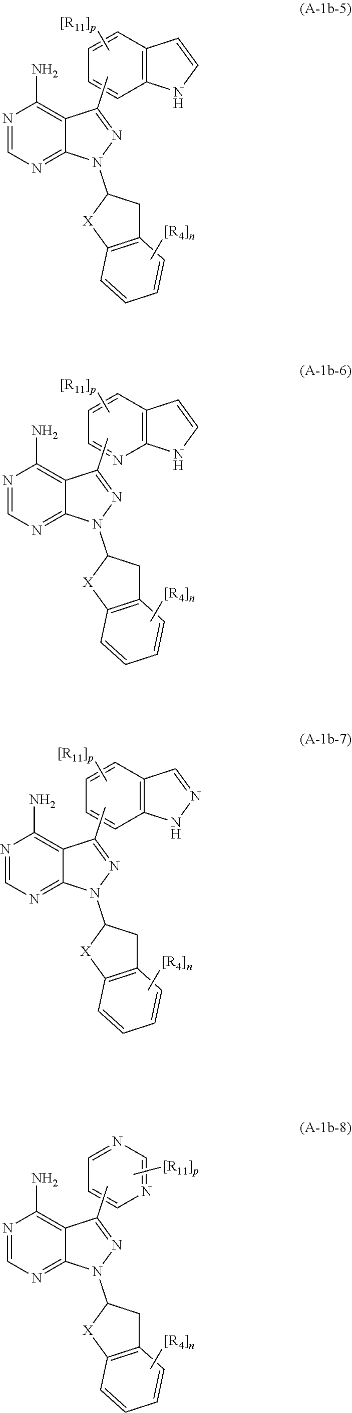 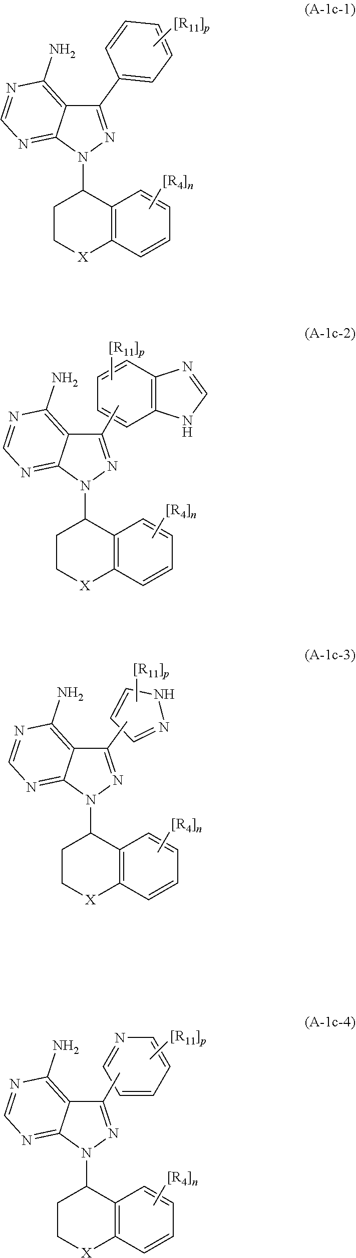 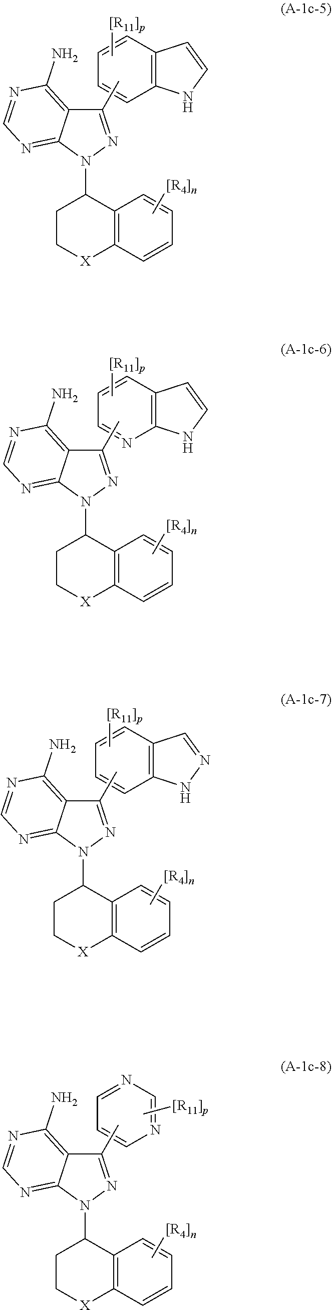 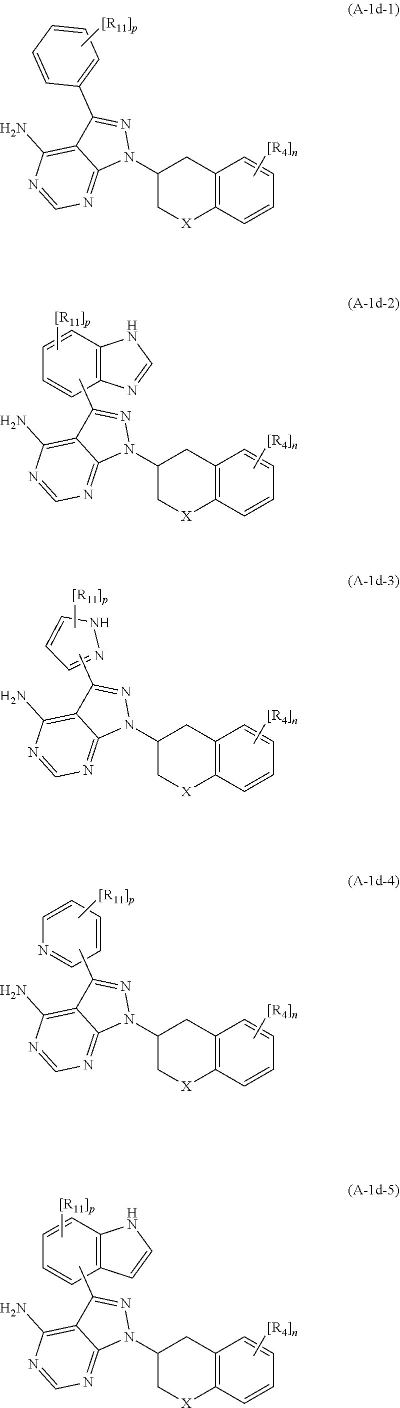 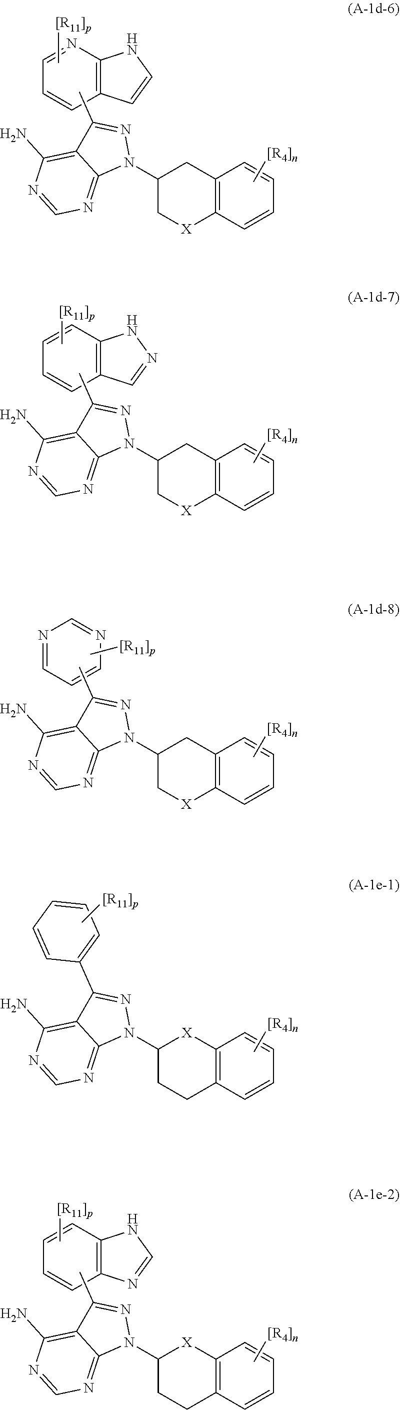 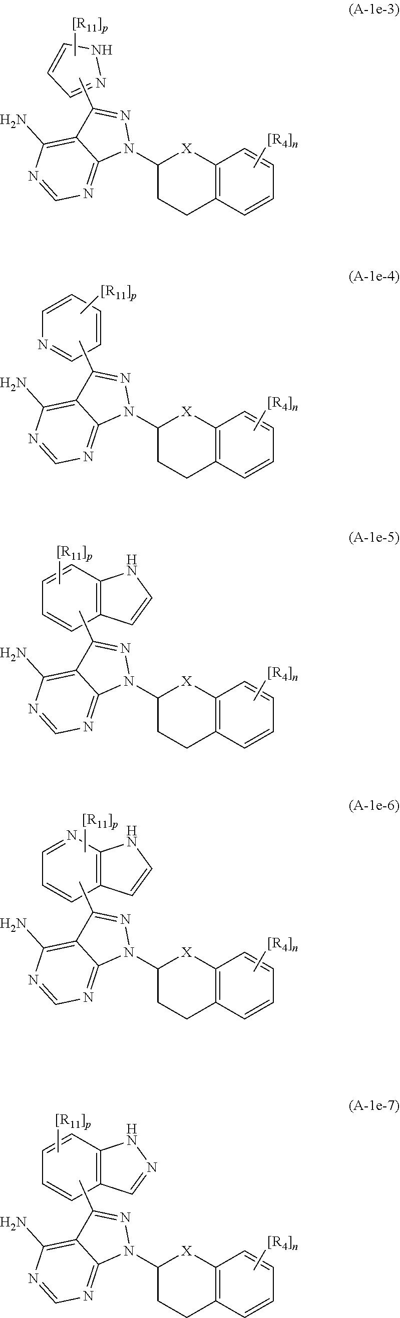 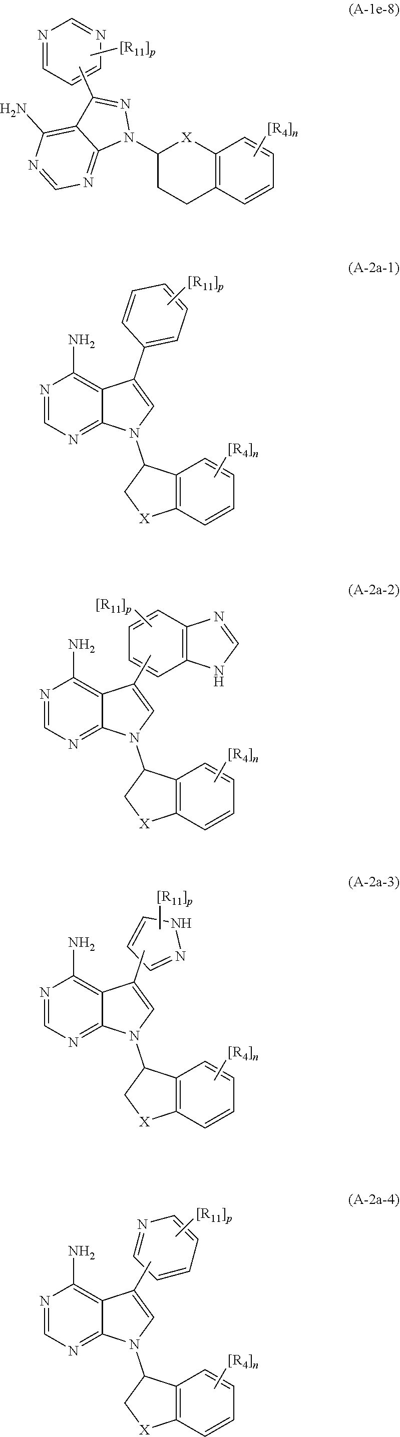 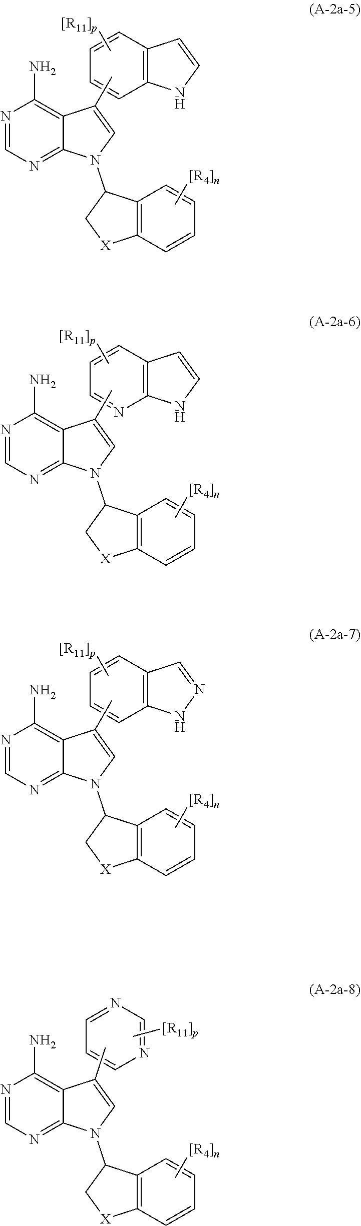 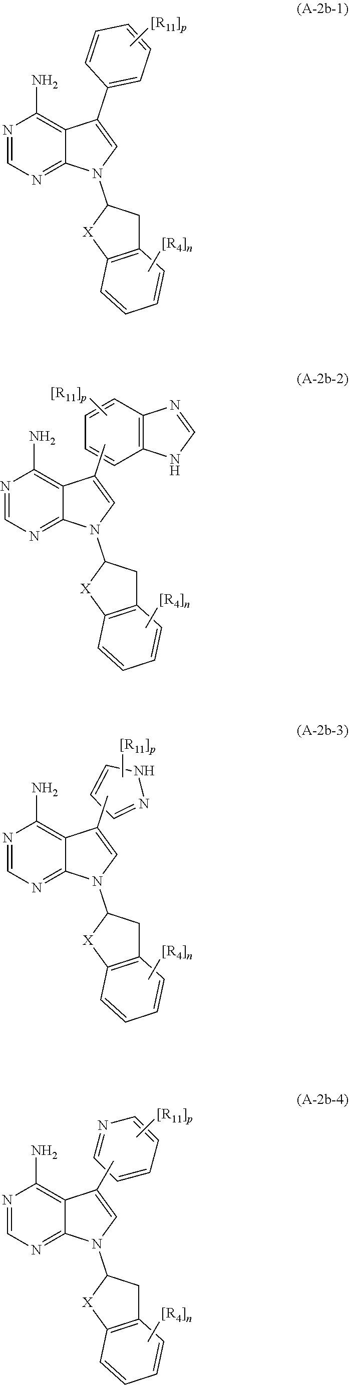 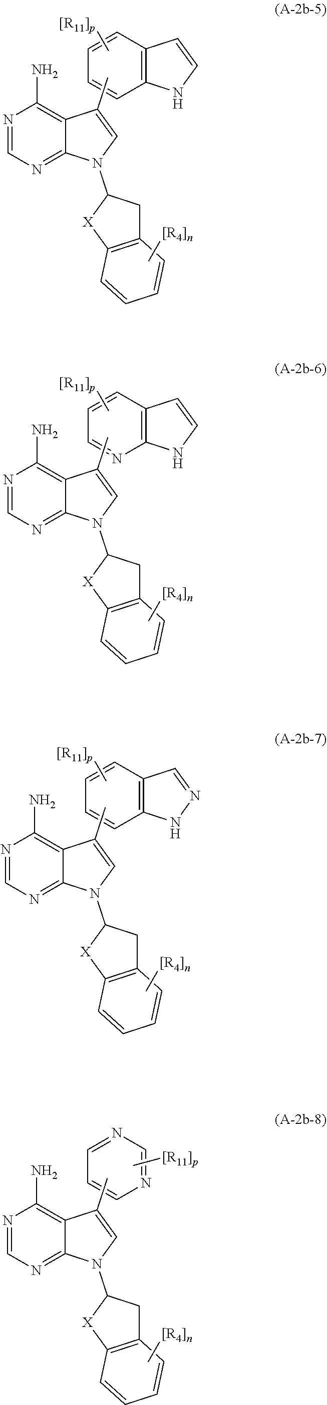 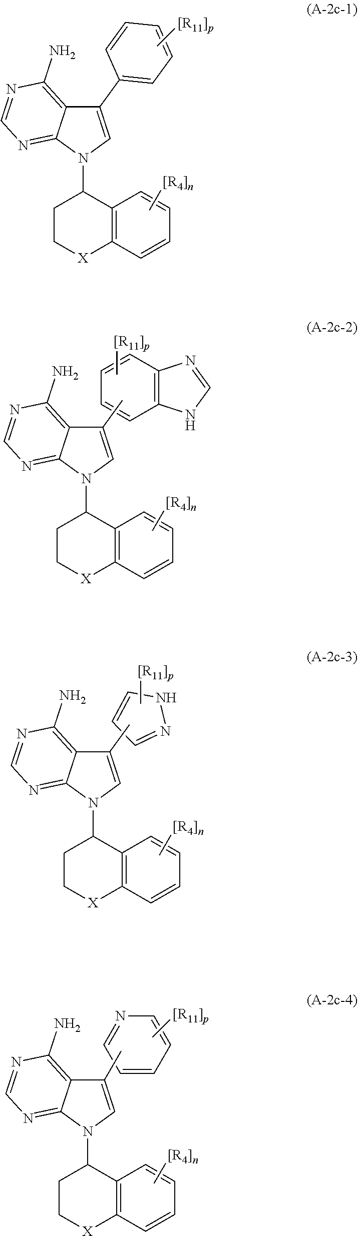 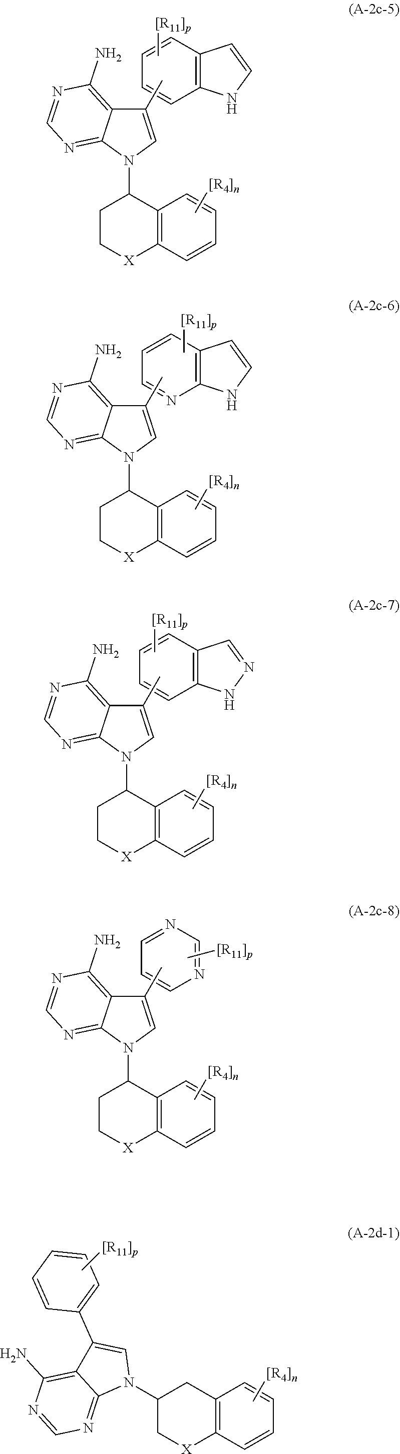 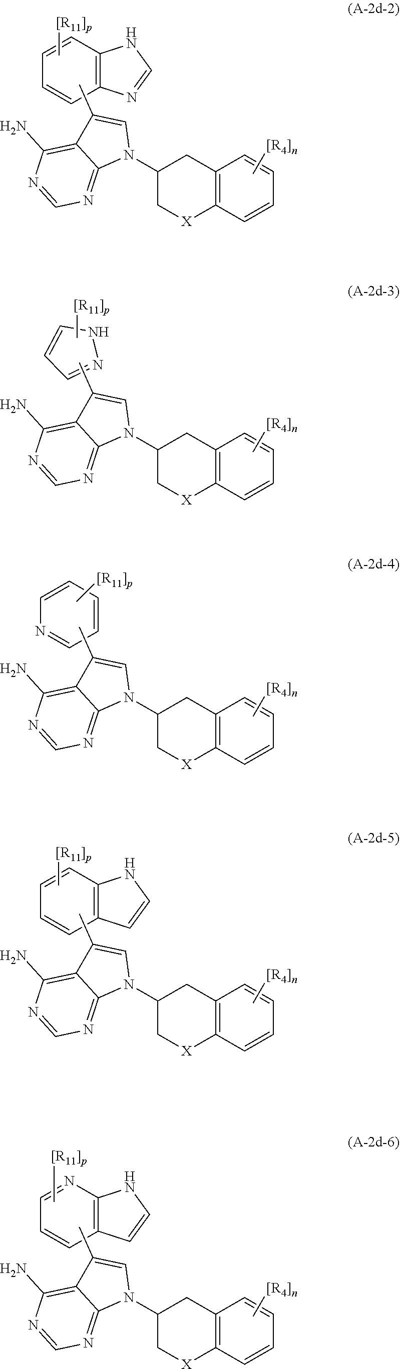 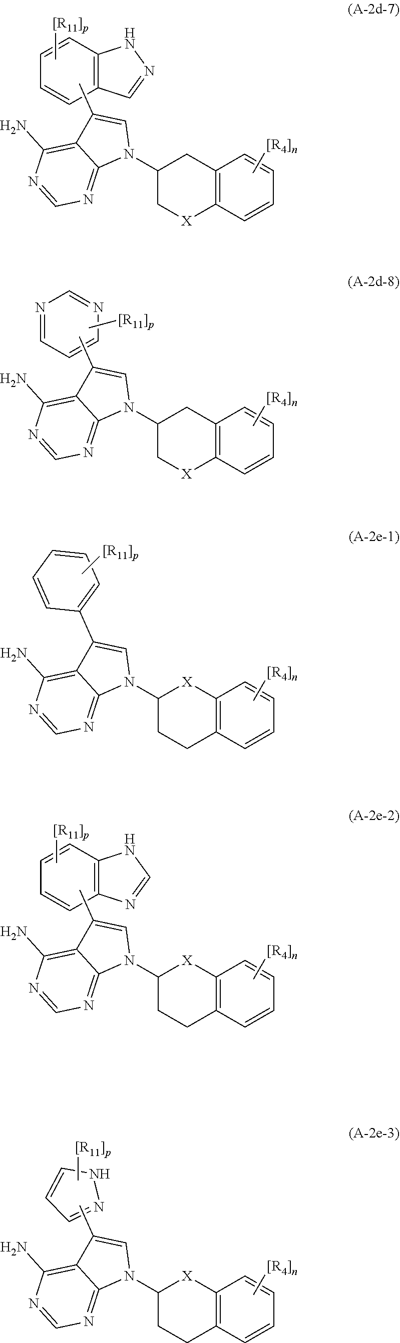 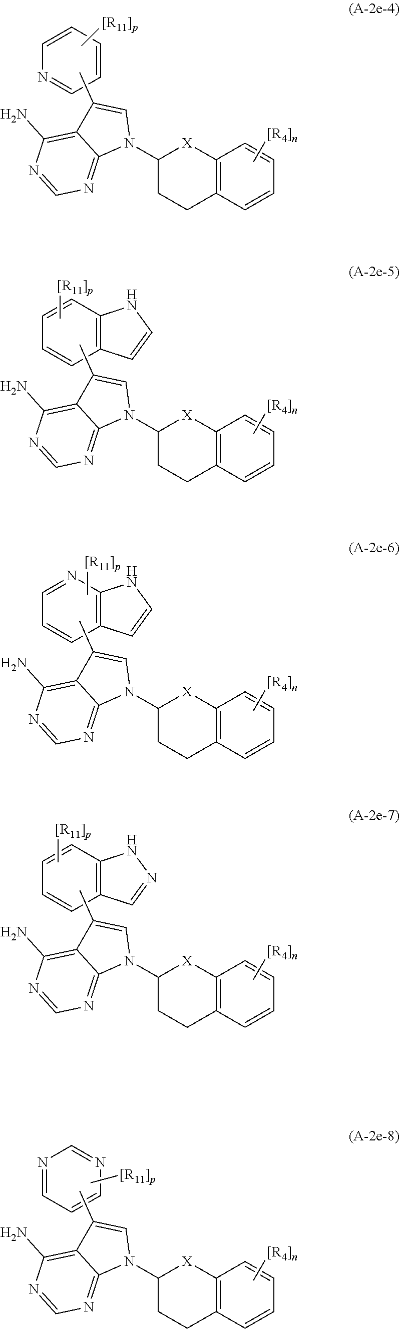 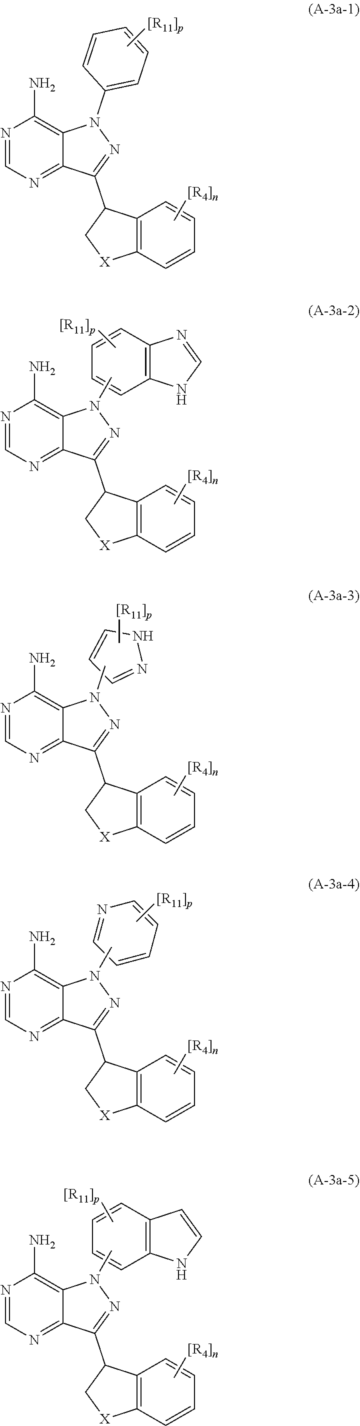 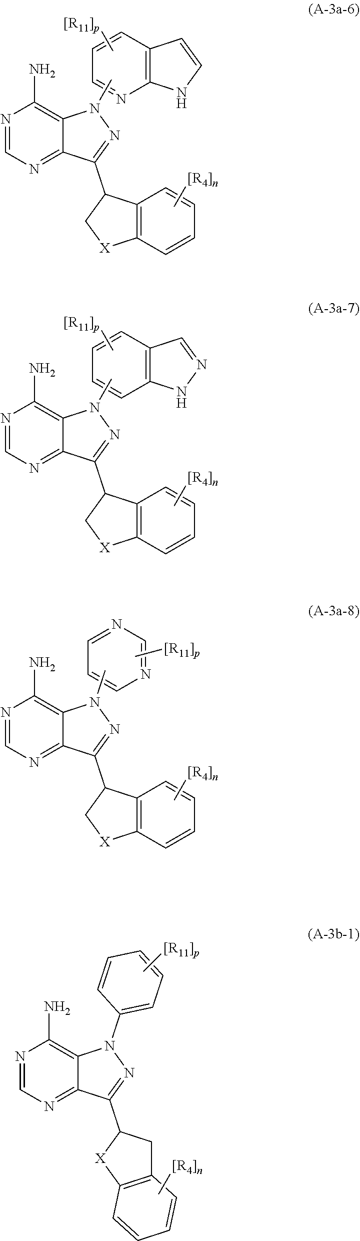 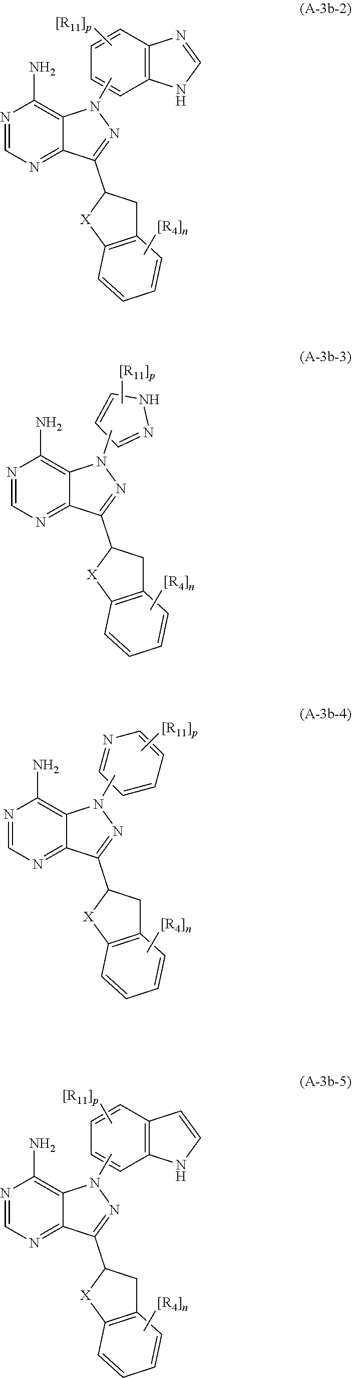 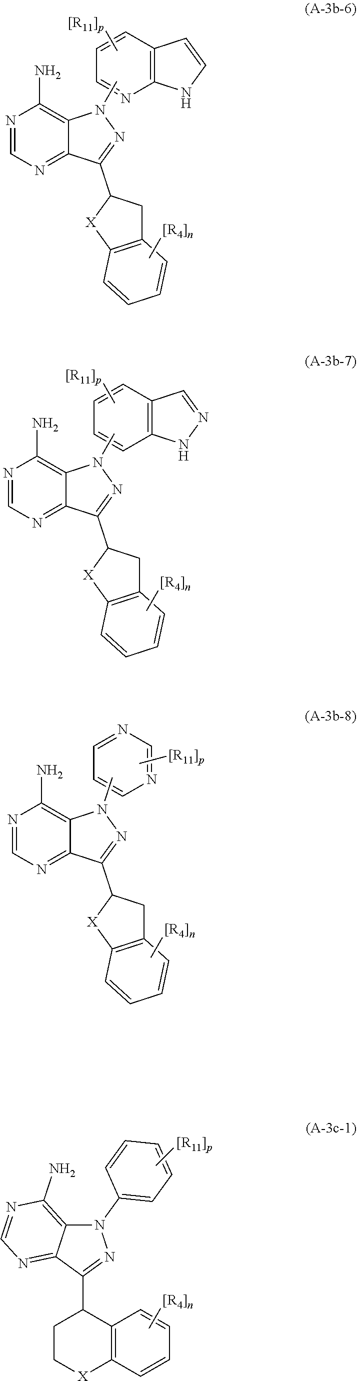 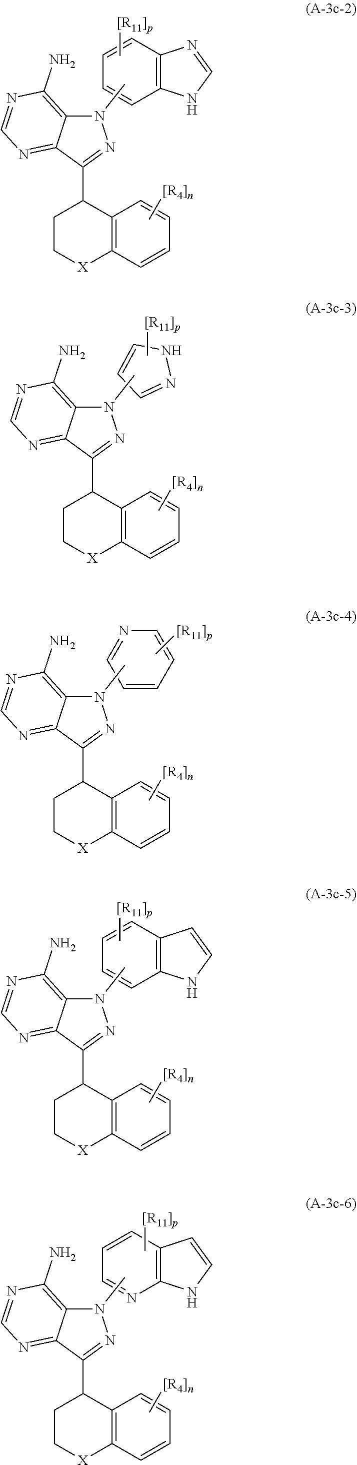 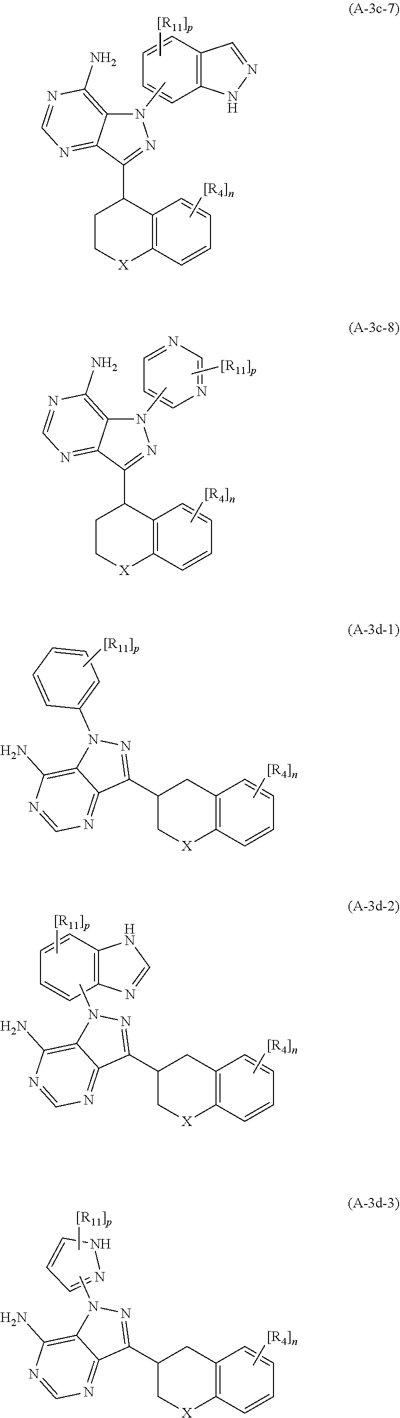 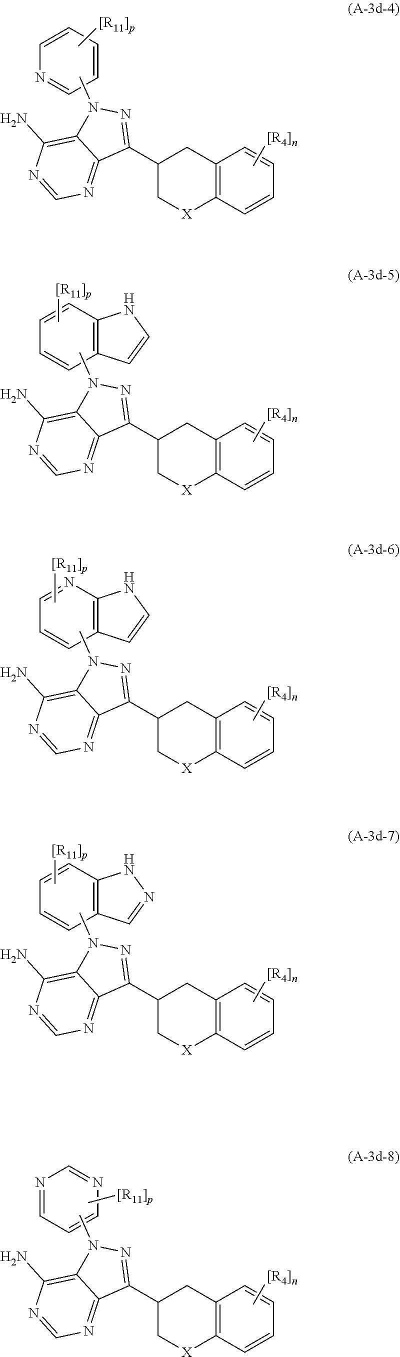 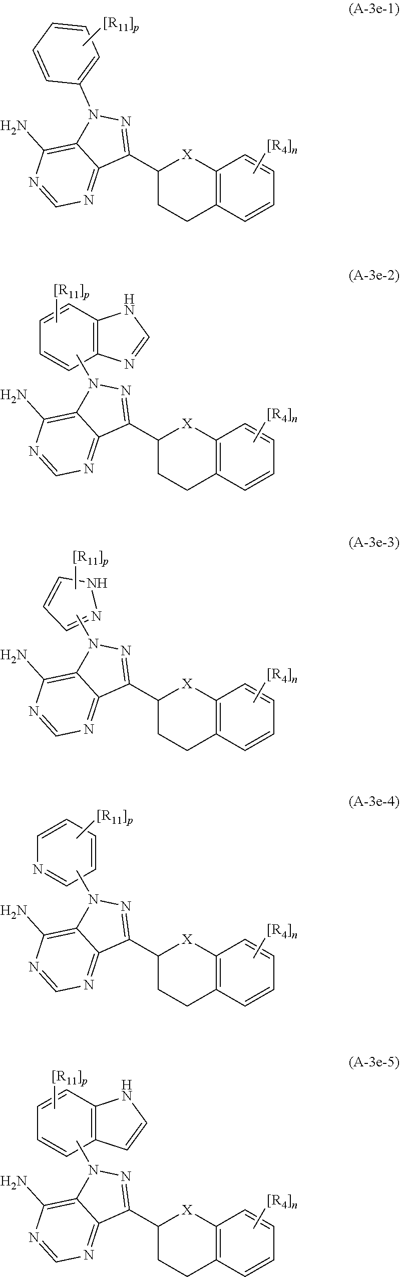 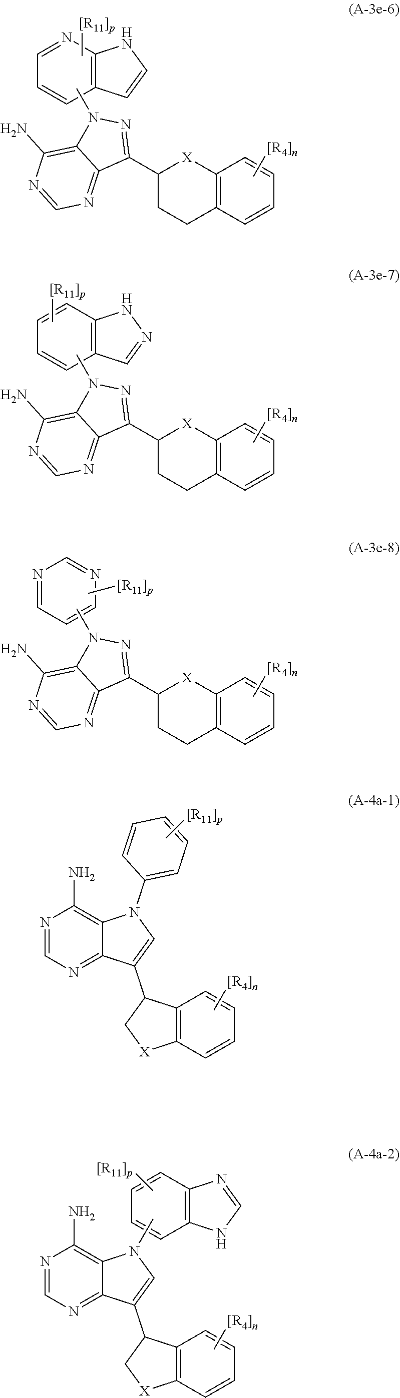 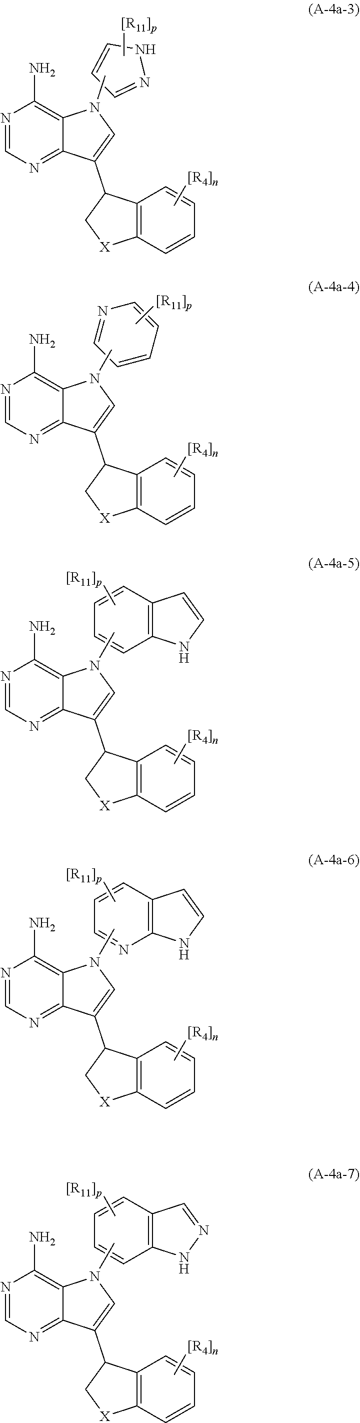 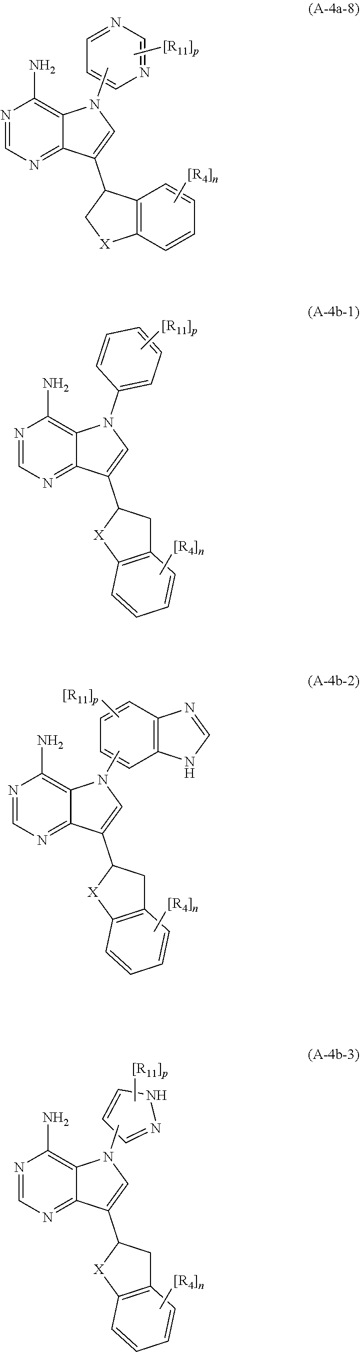 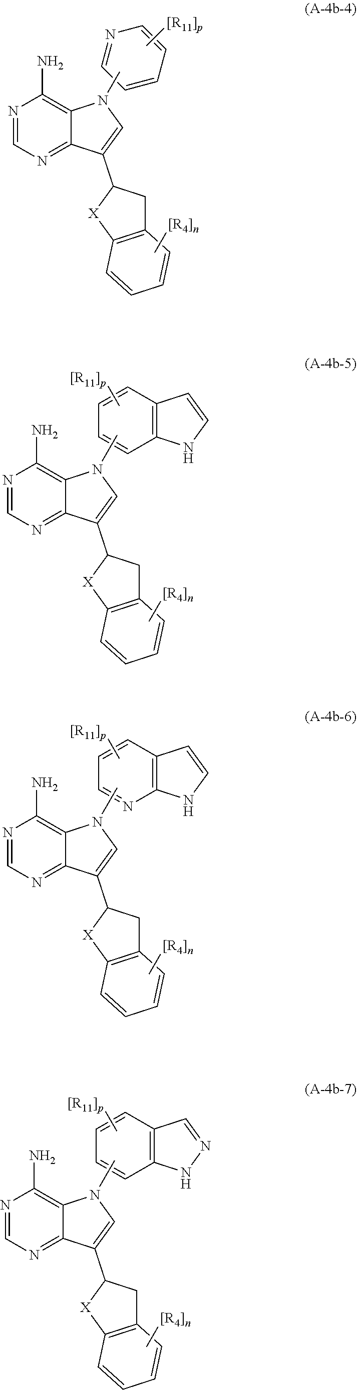 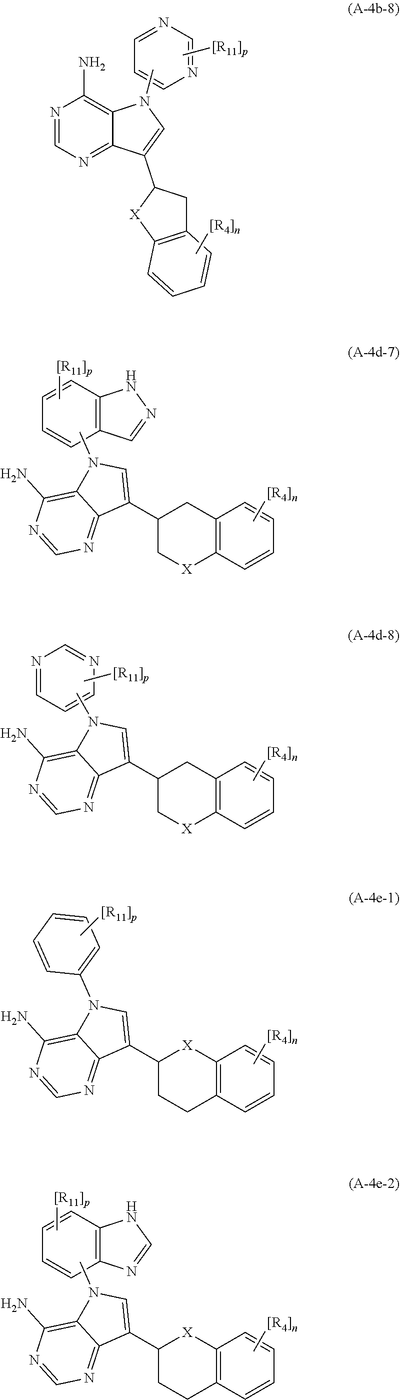 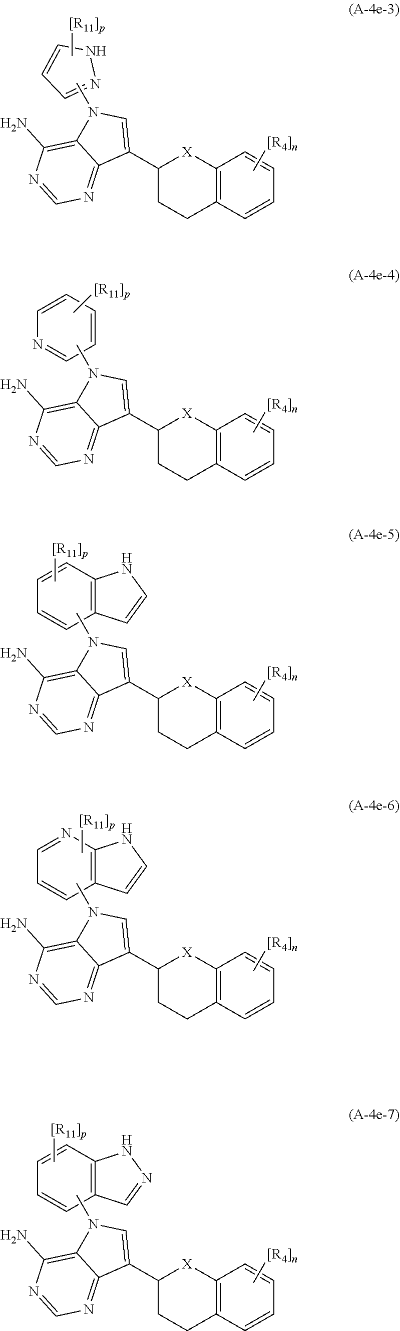 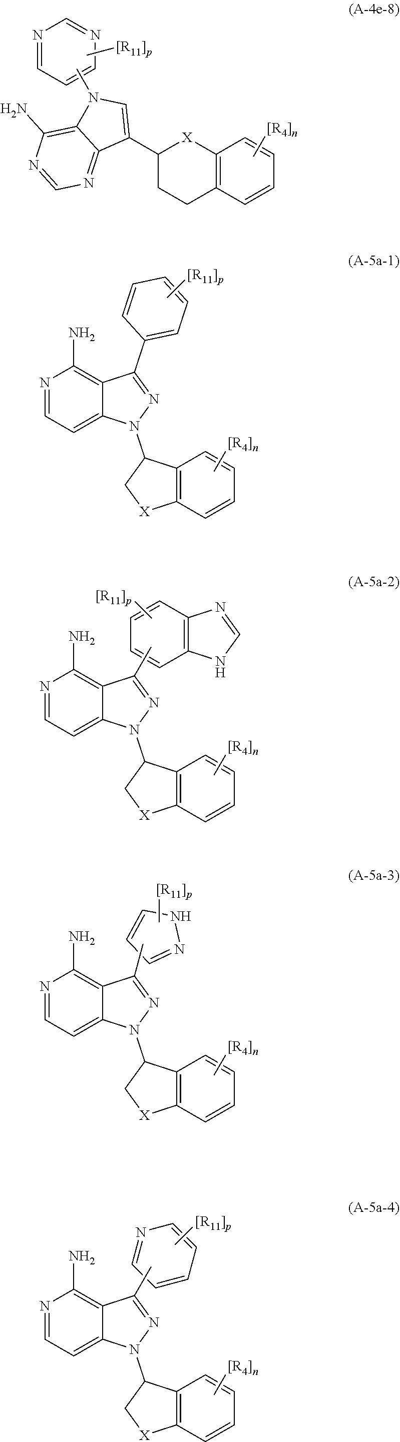 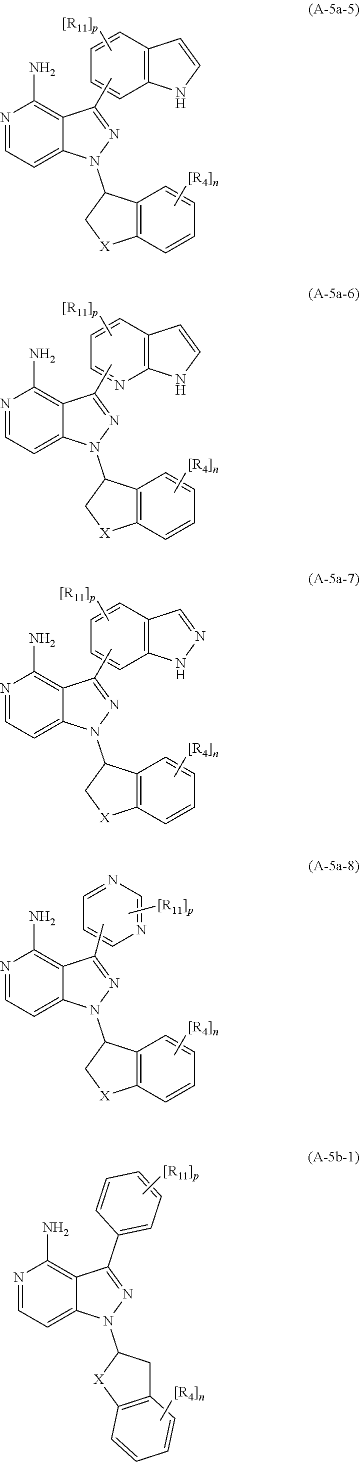 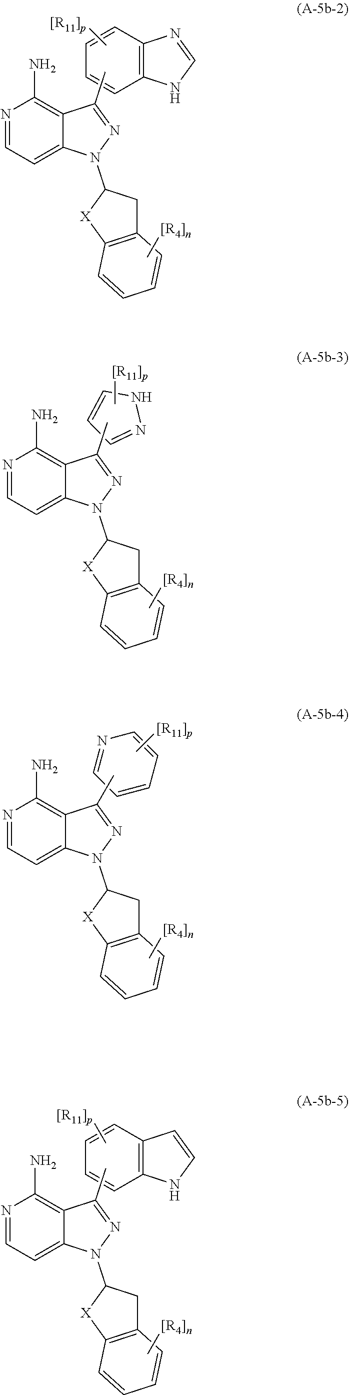 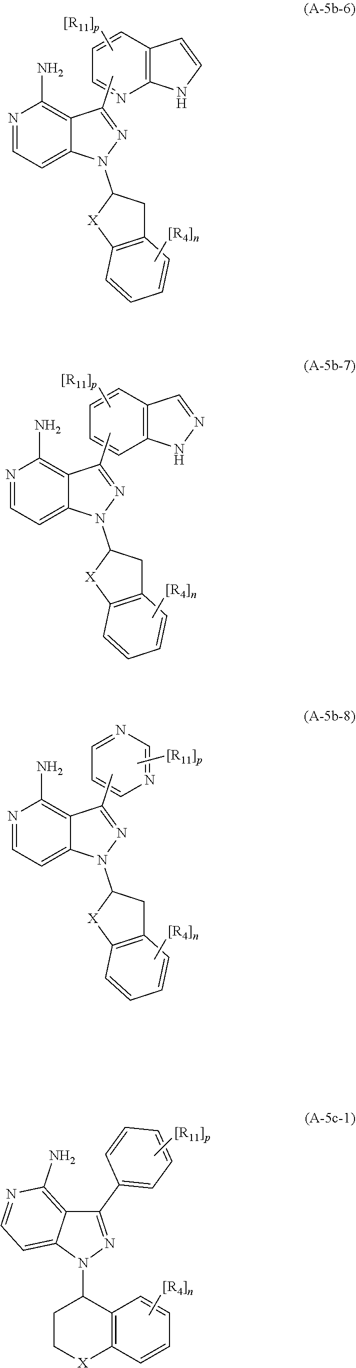 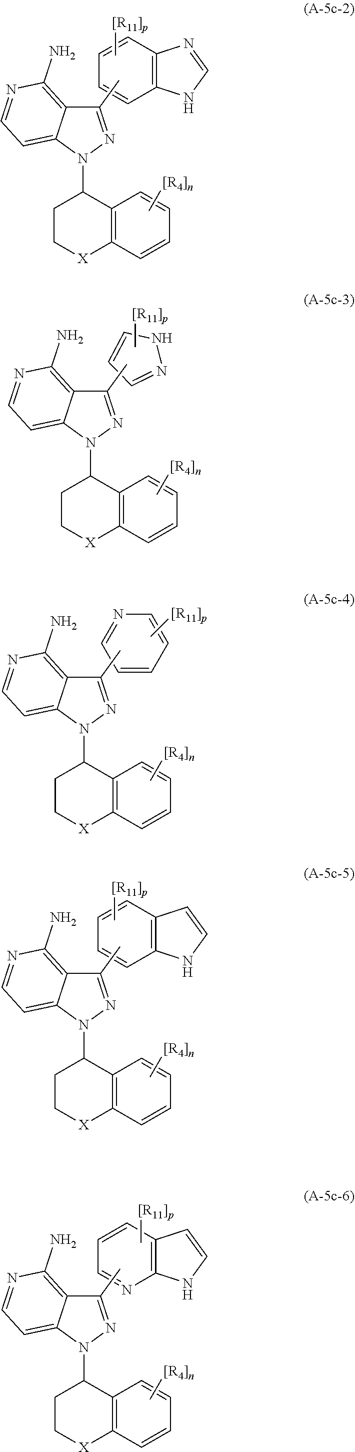 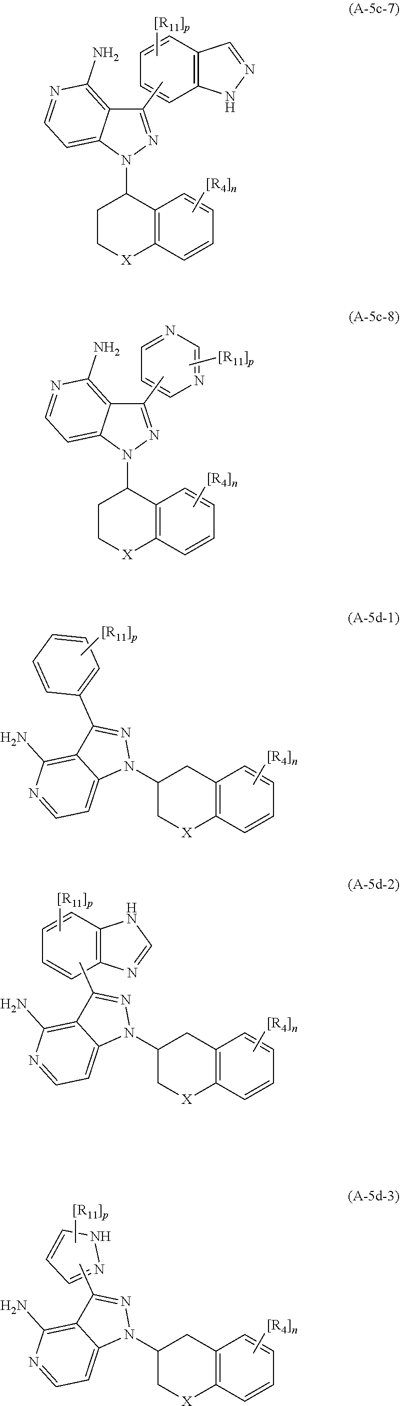 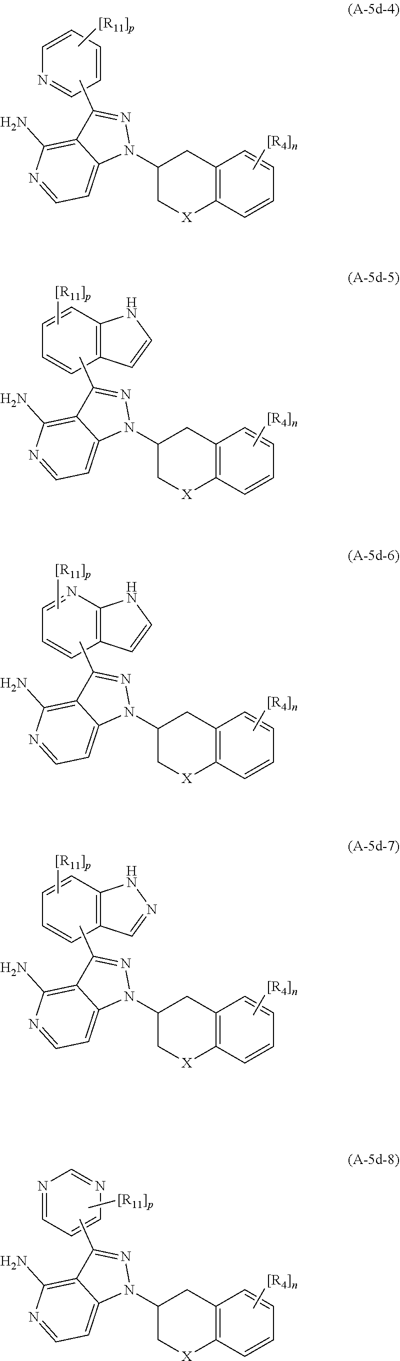 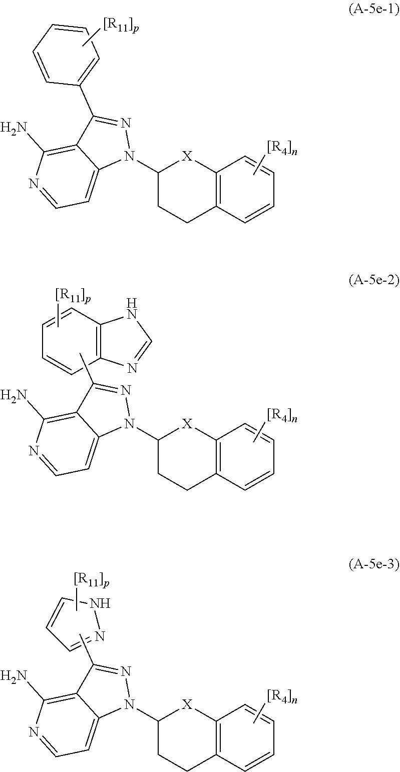 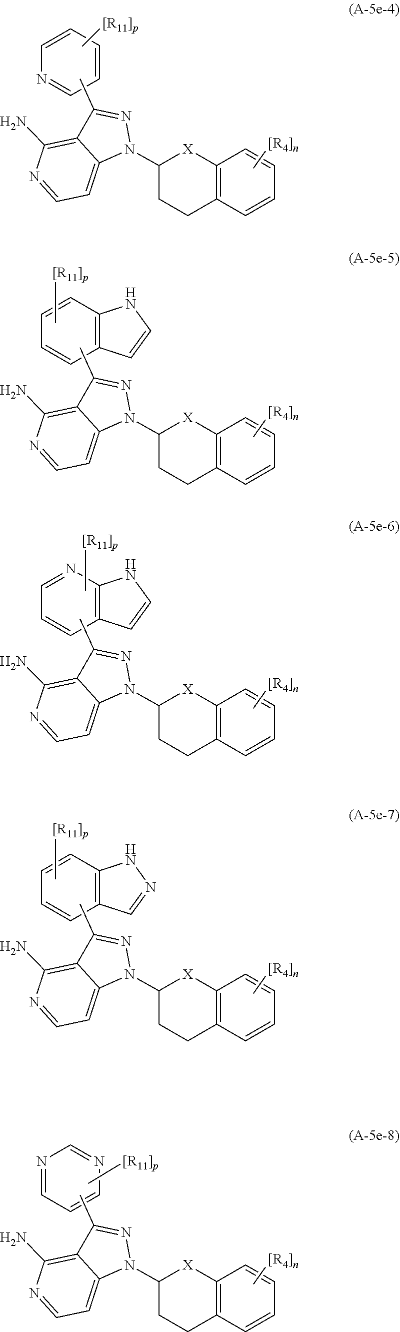 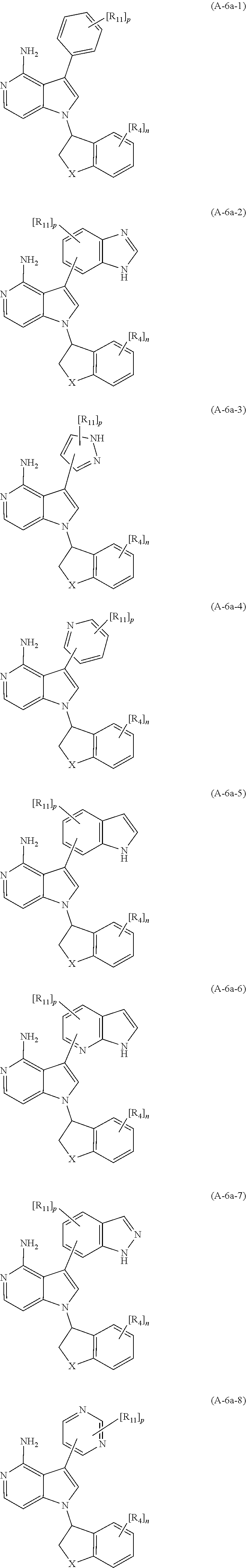 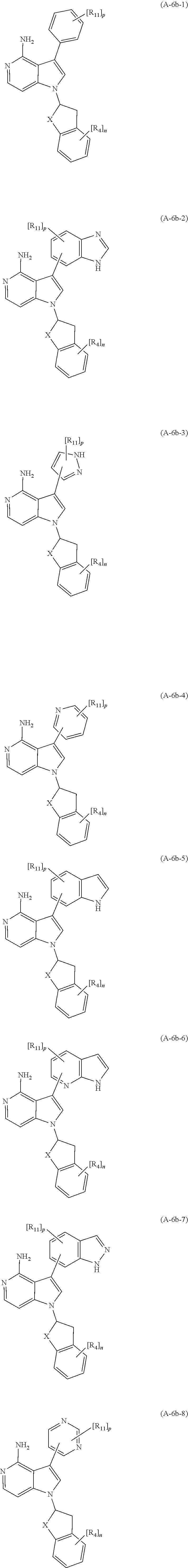 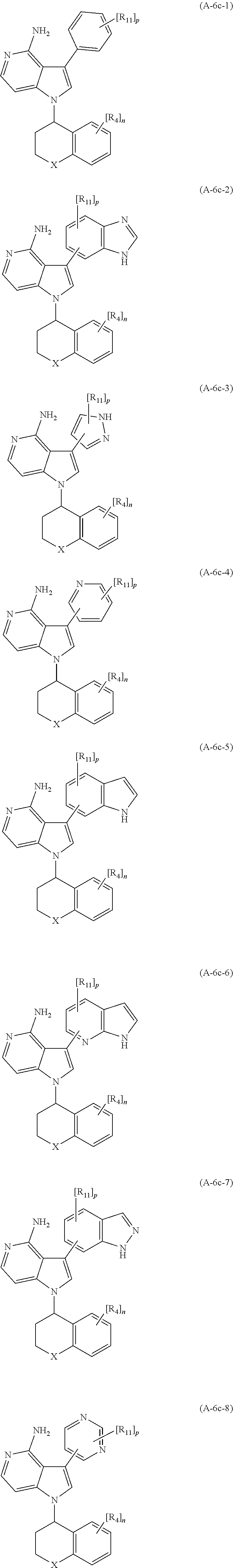 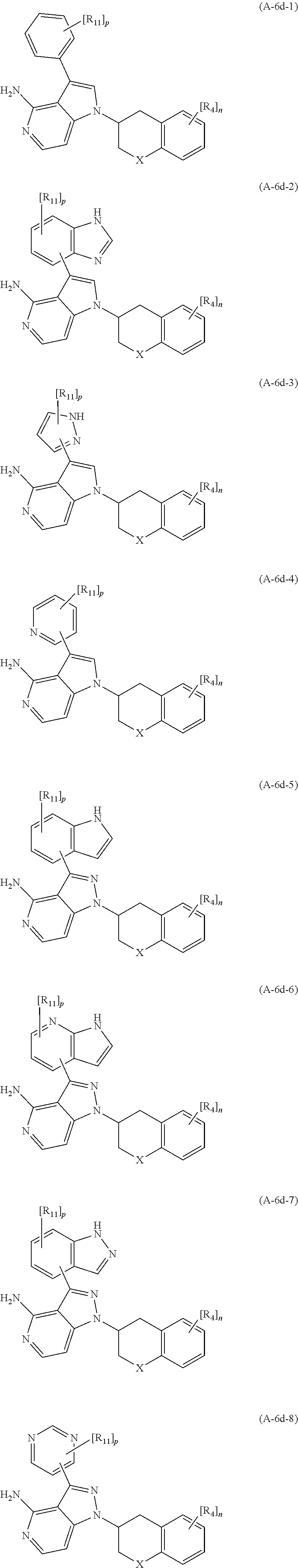 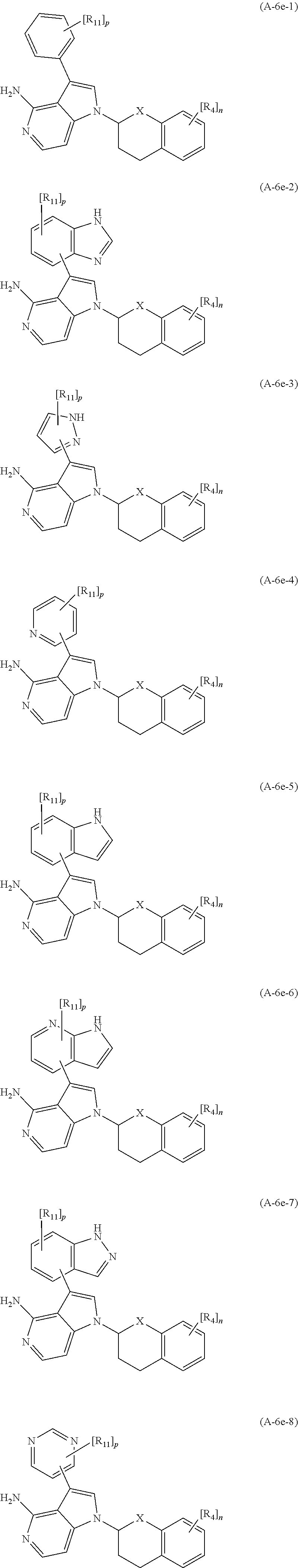 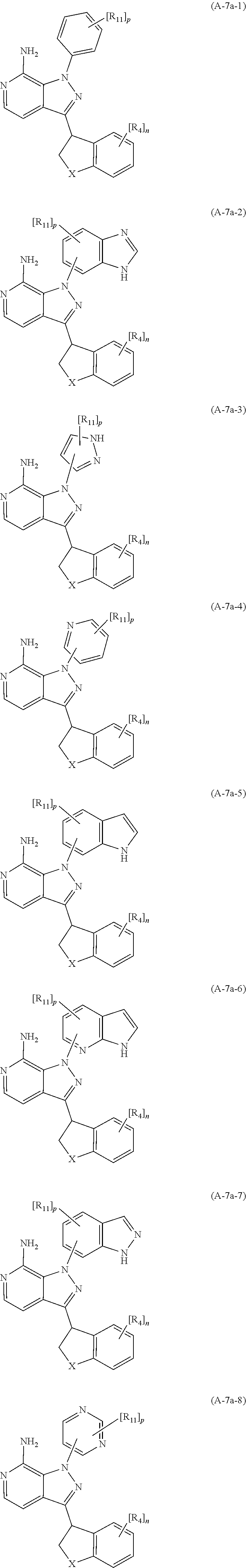 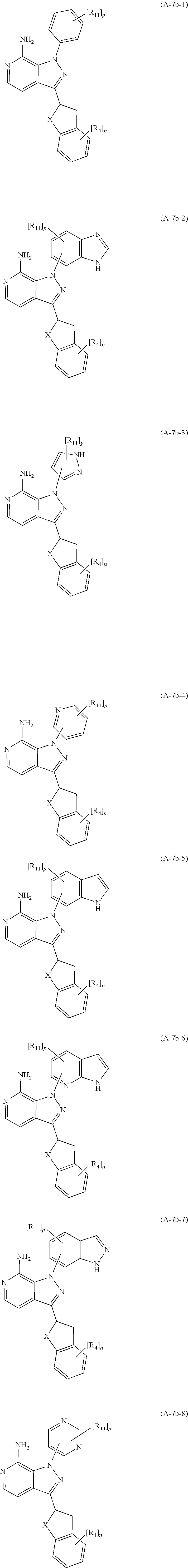 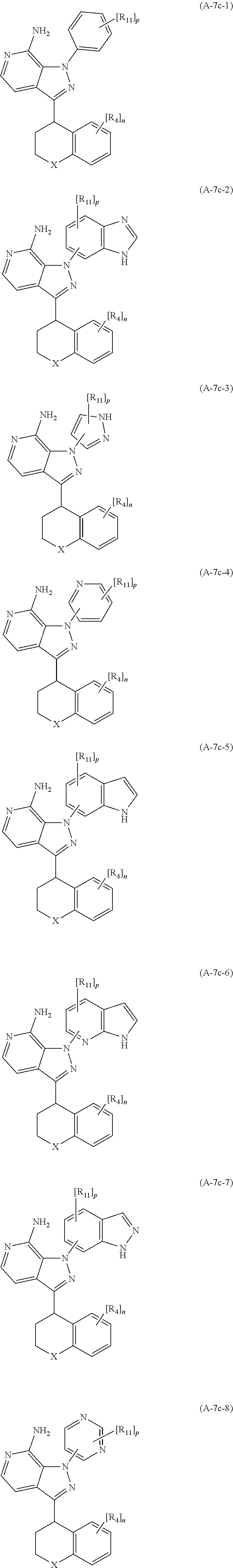 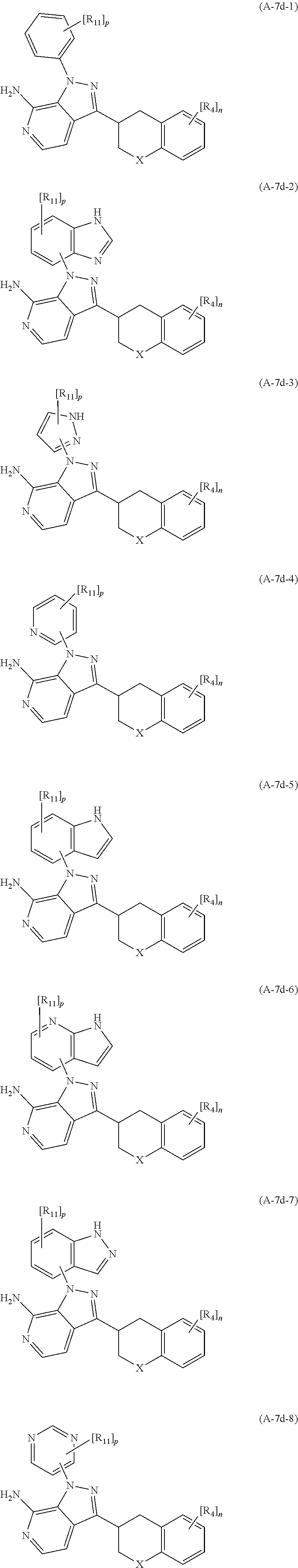 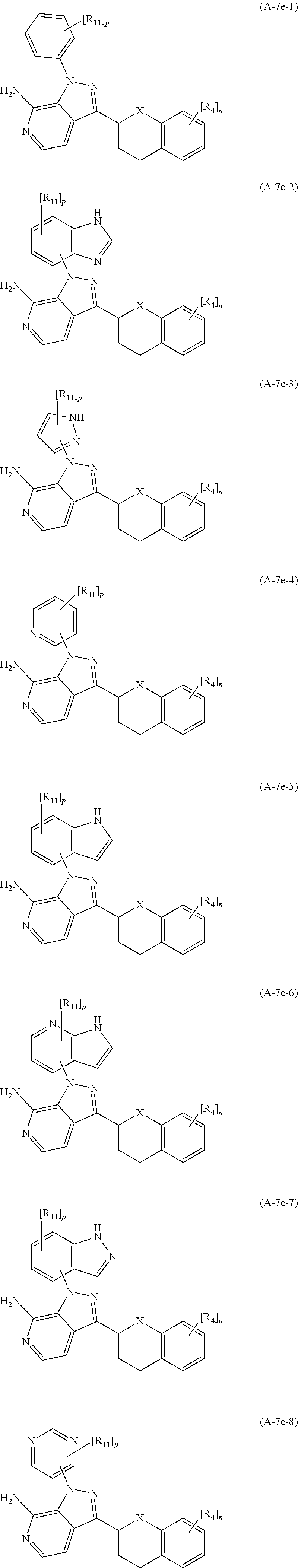 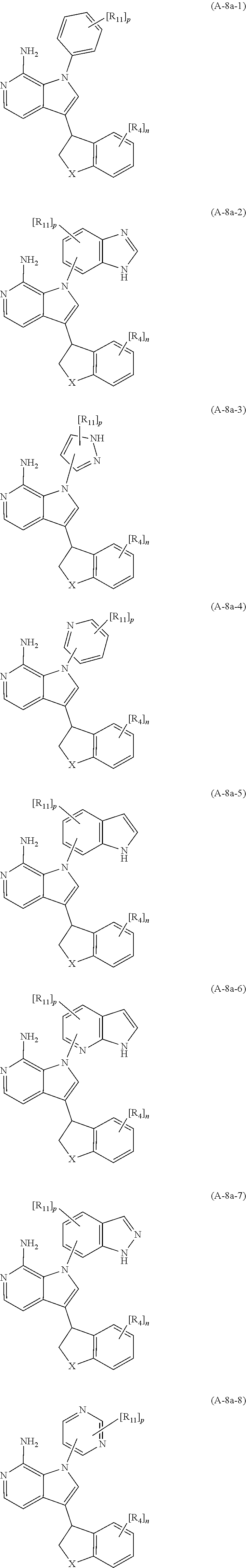 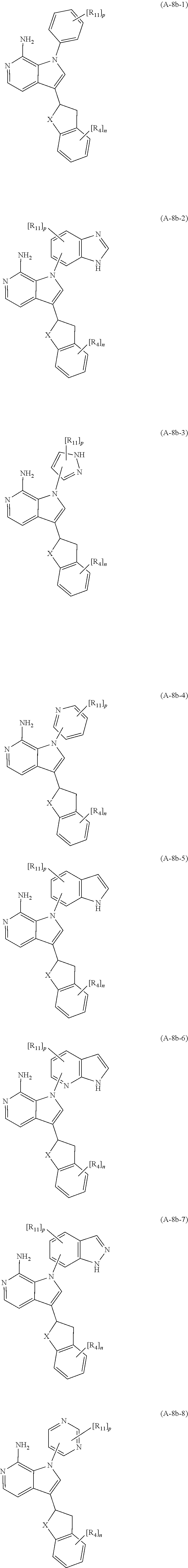 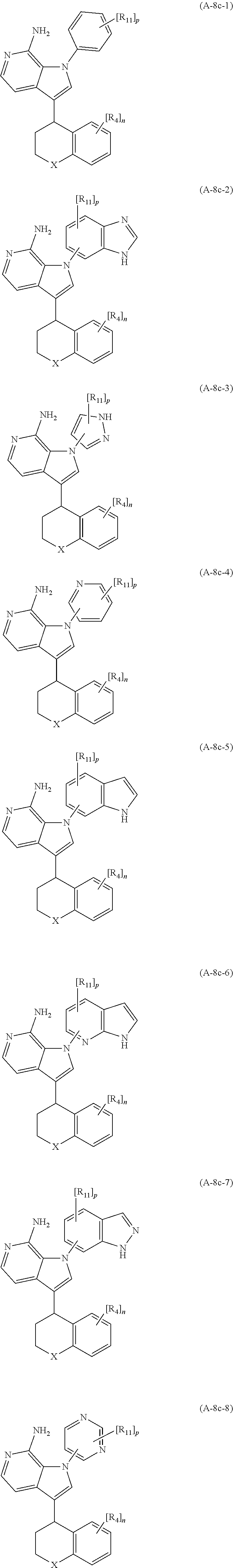 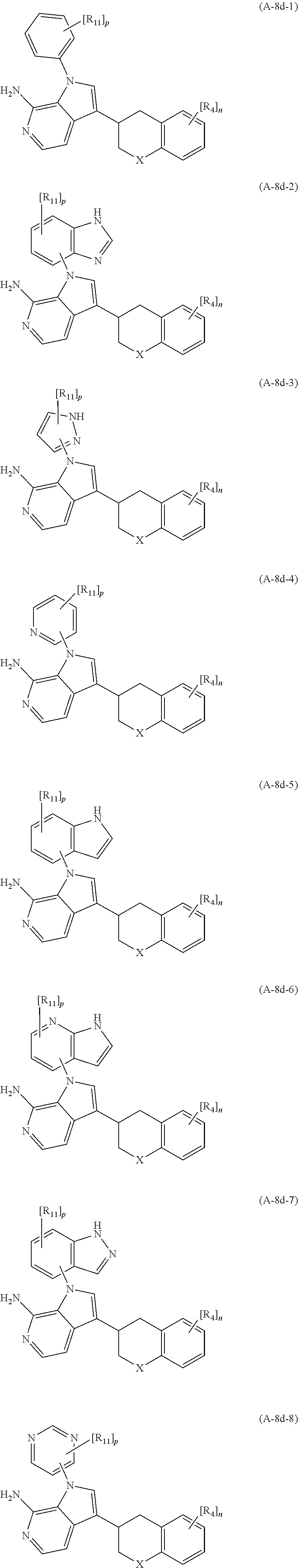 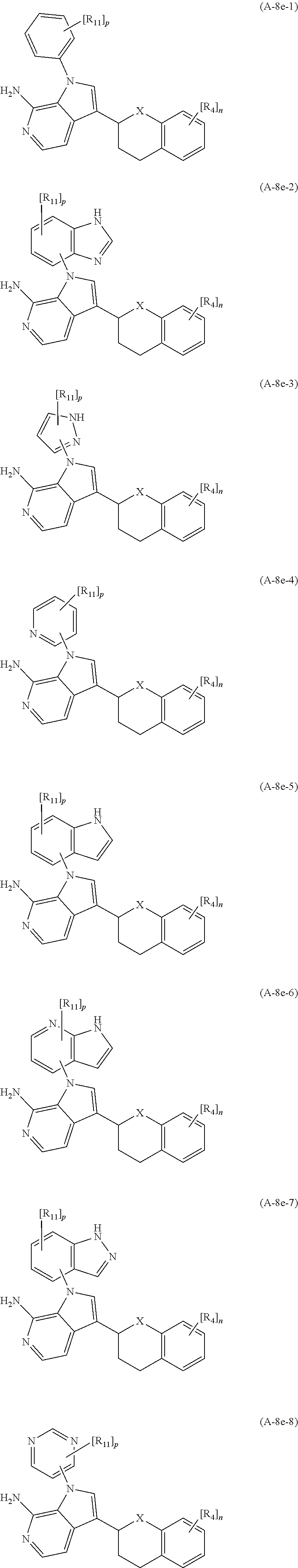 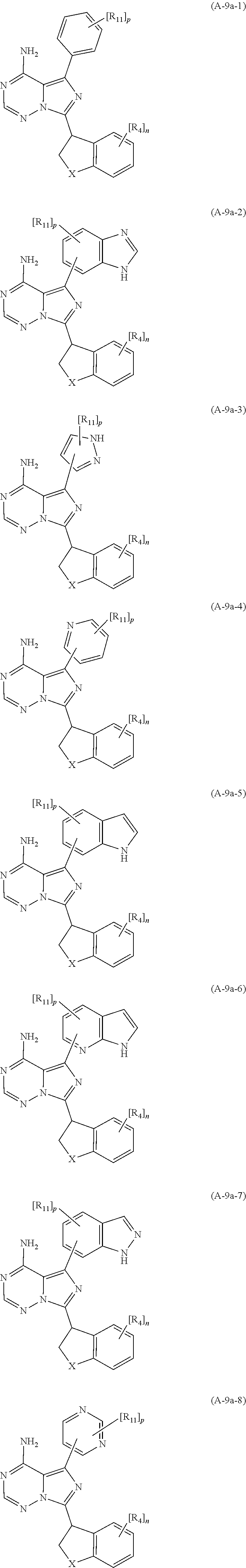 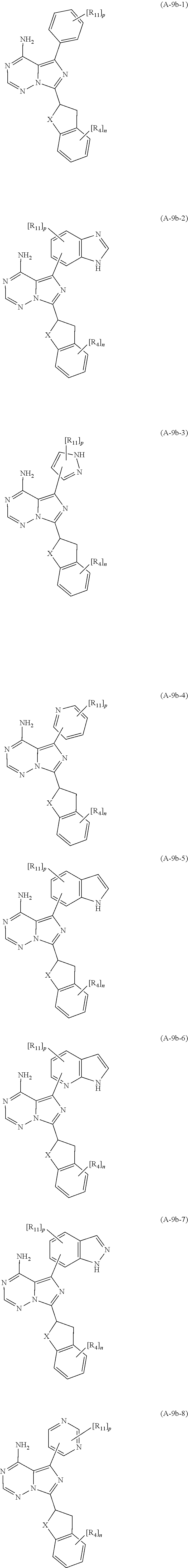 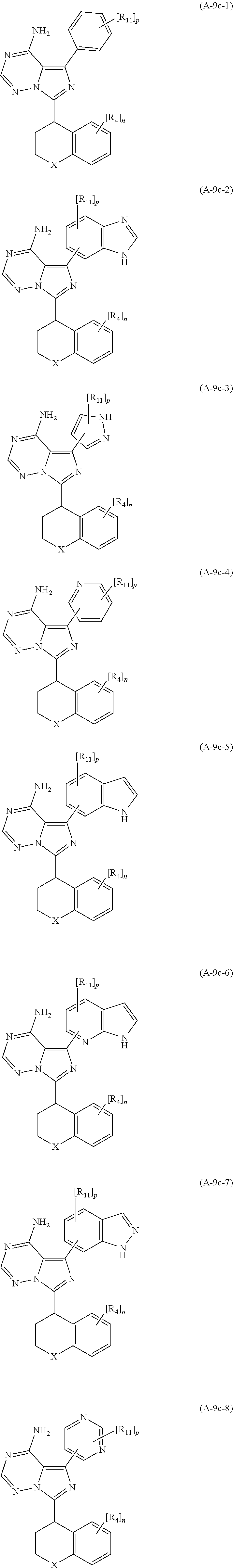 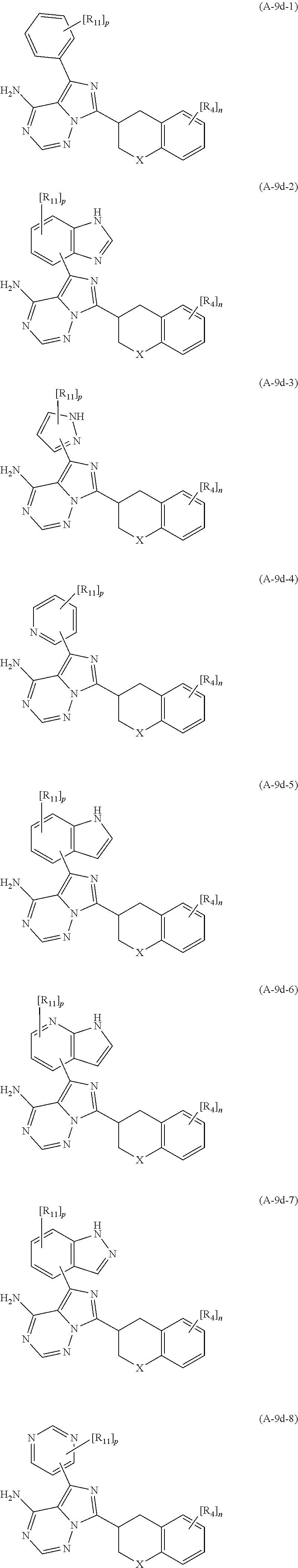 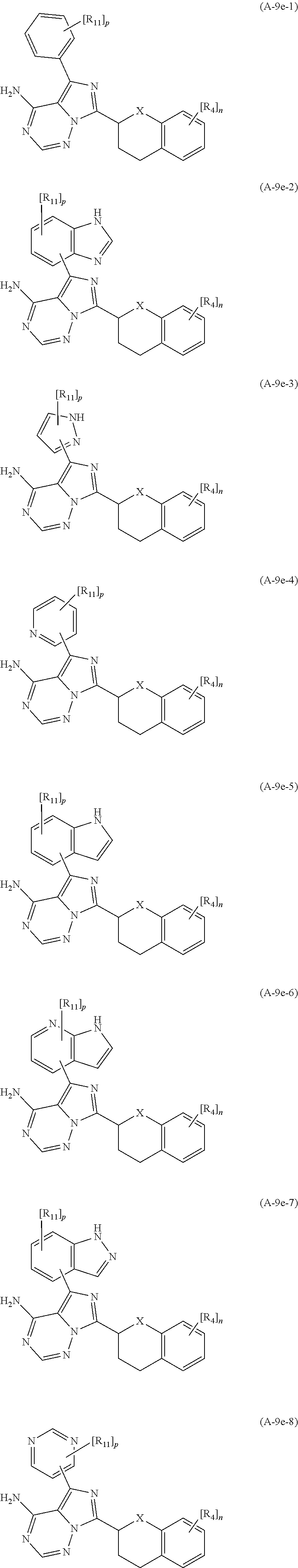 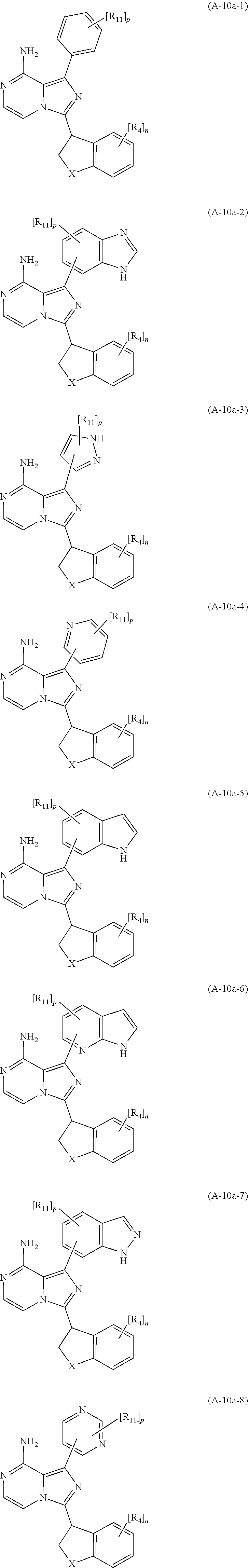 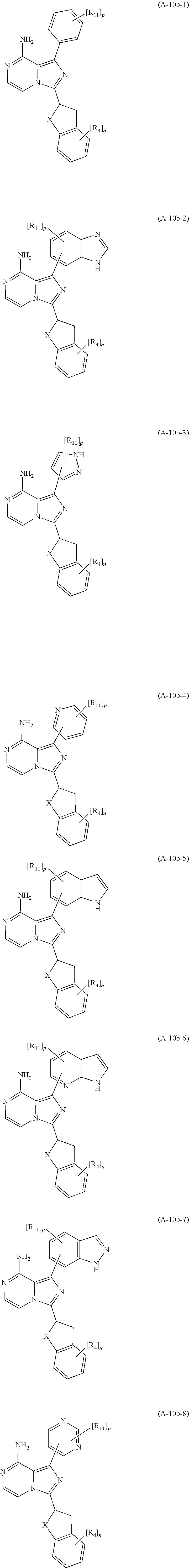 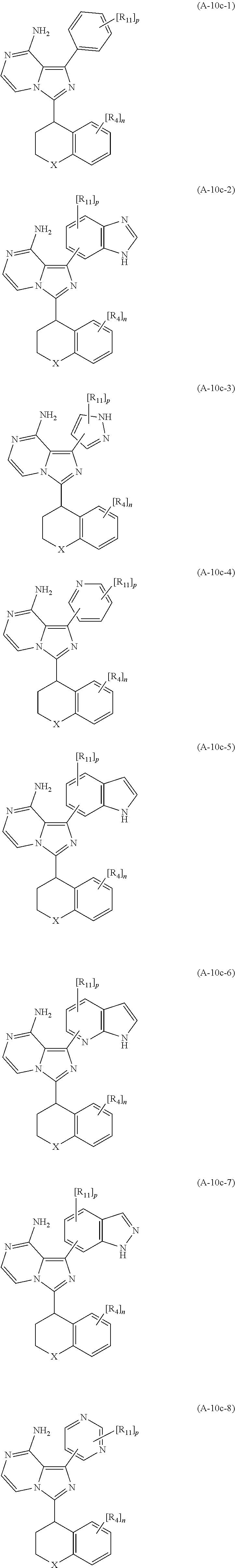 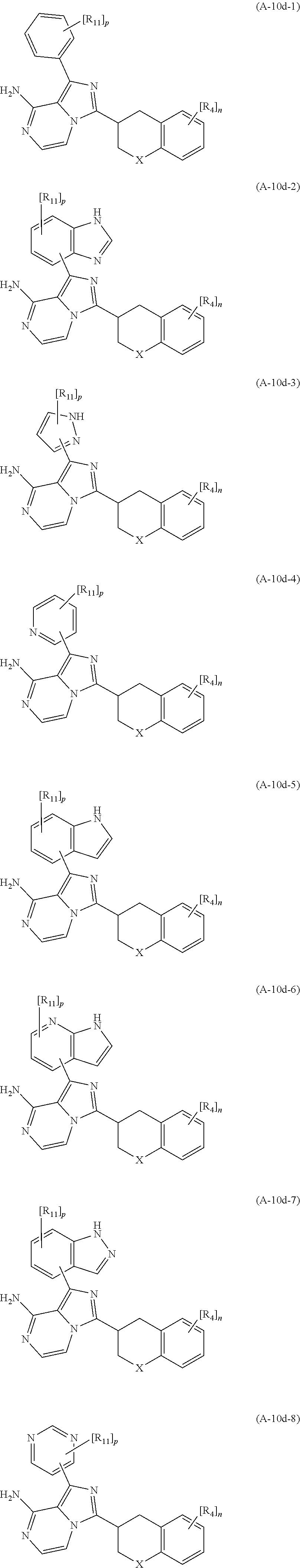 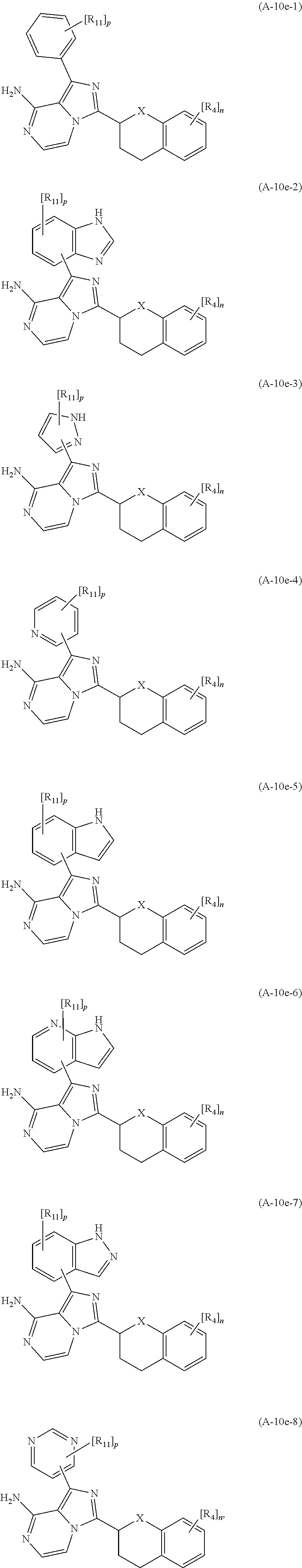
or a salt thereof; wherein R₄, X, n, R₅, R₆, R₇ and R₈, where present, are as described for formulae A-1a, A-1b, A-1c, A-1d, A-1e, A-2a, A-2b, A-2c, A-2d, A-2e, A-3a, A-3b, A-3c, A-3d, A-3e, A-4a, A-4b, A-4c, A-4d, A-4e, A-5a, A-5b, A-5c, A-5d, A-5e, A-6a, A-6b, A-6c, A-6d, A-6e, A-7a, A-7b, A-7c, A-7d, A-7e, A-8a, A-8b, A-8c, A-8d, A-8e, A-9a, A-9b, A-9c, A-9d, A-9e, A-10a, A-10b, A-10c, A-10d, and A-10e;
wherein each R₁₁, where present, is independently H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₃-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ or —NHR₅; and p is 0 or an integer from 1 to 3.

24. The compound of claim 1, wherein the compound is of the structure (B-1), (B-2), (B-3), or (B-4): 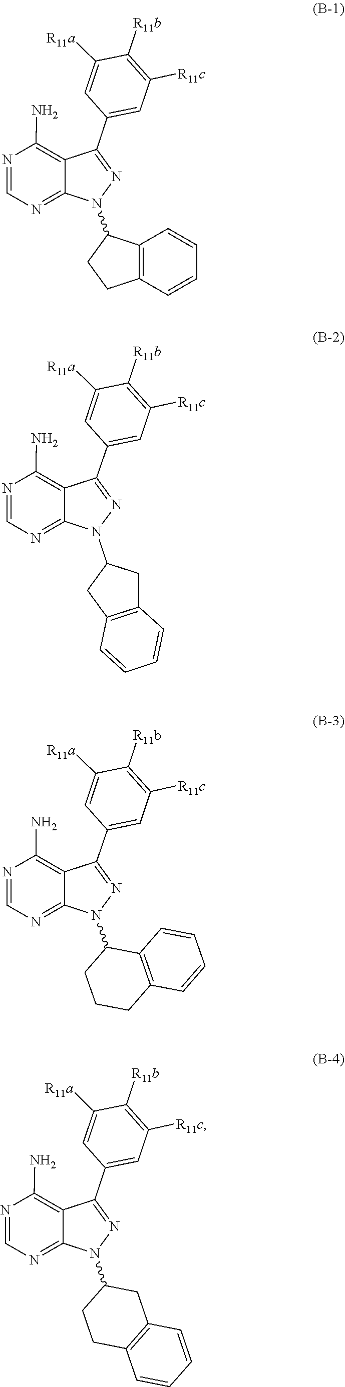
or a salt thereof, wherein each of R₁₁a, R₁₁b, and R₁₁c is independently selected from the group consisting of H, hydroxyl, nitro, cyano, halo, C₁-C₈ perhaloalkyl, substituted or unsubstituted C₁-C₈ alkyl, substituted or unsubstituted C₃-C₈ cycloalkyl, substituted or unsubstituted C₂-C₈ alkenyl, substituted or unsubstituted C₅-C₈ cycloalkenyl, substituted or unsubstituted C₂-C₈ alkynyl, substituted or unsubstituted aryl, substituted or unsubstituted heteroaryl, C₁-C₈ perhaloalkoxy, C₁-C₈ alkoxy, aryloxy, carboxyl, thiol, substituted or unsubstituted heterocyclyl, substituted or unsubstituted aralkyl, thioalkyl, substituted or unsubstituted amino, acylamino, aminoacyl, aminocarbonylamino, alkoxycarbonylamino, aminosulfonyl, sulfonylamino, sulfonyl, alkoxyalkylenecarbonyl, aminosulfonylalkylene, acyl, —R₅ and —NHR₅.

25. The compound of claim 1, wherein the compound is as described in Table 1 and Table 2, and pharmaceutically acceptable salts thereof.

26. A compound selected from the group consisting of:
4-(4-amino-1-(1,2,3,4-tetrahydronaphthalen-2-yl)-1H-pyrazolo[3,4-d]pyrimidin-3-yl)benzene-1,2-diol;
5-(4-amino-1-(2,3-dihydro-1H-inden-2-yl)-1H-pyrazolo[3,4-d]pyrimidin-3-yl)-2-bromophenol;
3-(4-amino-1-(2,3-dihydro-1H-inden-2-yl)-1H-pyrazolo[3,4-d]pyrimidin-3-yl)-5-fluorophenol;
1-(2,3-dihydro-1H-inden-1-yl)-3-(3-fluoro-5-methoxyphenyl)-1H-pyrazolo[3,4-d]pyrimidin-4-amine;
1-(2,3-dihydro-1H-inden-2-yl)-3-(3-fluoro-5-methoxyphenyl)-1H-pyrazolo[3,4-d]pyrimidin-4-amine;
3-(4-amino-1-(1,2,3,4-tetrahydronaphthalen-2-yl)-1H-pyrazolo[3,4-d]pyrimidin-3-yl)-5-fluorophenol;
3-(3-fluoro-5-methoxyphenyl)-1-(1,2,3,4-tetrahydronaphthalen-2-yl)-1H-pyrazolo[3,4-d]pyrimidin-4-amine;
5-(4-amino-1-(1,2,3,4-tetrahydronaphthalen-2-yl)-1H-pyrazolo[3,4-d]pyrimidin-3-yl)-2-bromophenol;
3-(1H-indazol-6-yl)-1-(1,2,3,4-tetrahydronaphthalen-2-yl)-1H-pyrazolo[3,4-d]pyrimidin-4-amine;
5-(4-amino-1-(2,3-dihydro-1H-inden-1-yl)-1H-pyrazolo[3,4-d]pyrimidin-3-yl)-2-fluorophenol;
3-(4-amino-1-(1,2,3,4-tetrahydronaphthalen-1-yl)-1H-pyrazolo[3,4-d]pyrimidin-3-yl)-5-fluorophenol;
5-(4-amino-1-(1,2,3,4-tetrahydronaphthalen-1-yl)-1H-pyrazolo[3,4-d]pyrimidin-3-yl)-2-fluorophenol;
3-(4-fluorophenyl)-1-(1,2,3,4-tetrahydronaphthalen-2-yl)-1H-pyrazolo[3,4-d]pyrimidin-4-amine;
3-(3-fluoro-4-methoxyphenyl)-1-(1,2,3,4-tetrahydronaphthalen-2-yl)-1H-pyrazolo[3,4-d]pyrimidin-4-amine;
3-(6-methoxypyridin-3-yl)-1-(1,2,3,4-tetrahydronaphthalen-2-yl)-1H-pyrazolo[3,4-d]pyrimidin-4-amine;
3-(1H-pyrrolo[2,3-b]pyridin-5-yl)-1-(1,2,3,4-tetrahydronaphthalen-2-yl)-1H-pyrazolo[3,4-d]pyrimidin-4-amine;
3-(3,4-dimethoxyphenyl)-1-(1,2,3,4-tetrahydronaphthalen-2-yl)-1H-pyrazolo[3,4-d]pyrimidin-4-amine;
3-(1H-indol-6-yl)-1-(1,2,3,4-tetrahydronaphthalen-2-yl)-1H-pyrazolo[3,4-d]pyrimidin-4-amine;
3-(1H-benzo[d]imidazol-5-yl)-1-(1,2,3,4-tetrahydronaphthalen-2-yl)-1H-pyrazolo[3,4-d]pyrimidin-4-amine;
3-(1H-pyrazol-4-yl)-1-(1,2,3,4-tetrahydronaphthalen-2-yl)-1H-pyrazolo[3,4-d]pyrimidin-4-amine;
4-(4-amino-1-(1,2,3,4-tetrahydronaphthalen-2-yl)-1H-pyrazolo[3,4-d]pyrimidin-3-yl)phenol;
4-(4-amino-1-(1,2,3,4-tetrahydronaphthalen-2-yl)-1H-pyrazolo[3,4-d]pyrimidin-3-yl)-2-fluorophenol;
3-(5-fluoro-6-methoxypyridin-3-yl)-1-(1,2,3,4-tetrahydronaphthalen-2-yl)-1H-pyrazolo[3,4-d]pyrimidin-4-amine;
5-(4-amino-1-(1,2,3,4-tetrahydronaphthalen-2-yl)-1H-pyrazolo[3,4-d]pyrimidin-3-yl)pyridin-2-ol;
3-(6-chloropyridin-3-yl)-1-(1,2,3,4-tetrahydronaphthalen-2-yl)-1H-pyrazolo[3,4-d]pyrimidin-4-amine;
5-(4-amino-1-(1,2,3,4-tetrahydronaphthalen-2-yl)-1H-pyrazolo[3,4-d]pyrimidin-3-yl)-2-fluorophenol;
3-(4-amino-1-(1,2,3,4-tetrahydronaphthalen-2-yl)-1H-pyrazolo[3,4-d]pyrimidin-3-yl)benzamide;
4-(4-amino-1-(1,2,3,4-tetrahydronaphthalen-2-yl)-1H-pyrazolo[3,4-d]pyrimidin-3-yl)benzamide;
5-(4-amino-1-(2,3-dihydro-1H-inden-2-yl)-1H-pyrazolo[3,4-d]pyrimidin-3-yl)-2-fluorophenol;
3-(4-amino-1-(2,3-dihydro-1H-inden-1-yl)-1H-pyrazolo[3,4-d]pyrimidin-3-yl)-5-fluorophenol;
1-(3-(4-amino-3-(3-fluoro-5-hydroxyphenyl)-1H-pyrazolo[3,4-d]pyrimidin-1-yl)-6-methyl-3,4-dihydroquinolin-1(2H)-yl)prop-2-en-1-one;
1-(3-(4-amino-3-(5-fluoro-6-methoxypyridin-3-yl)-1H-pyrazolo[3,4-d]pyrimidin-1-yl)-6-methyl-3,4-dihydroquinolin-1(2H)-yl)prop-2-en-1-one;
3-(3,4-dimethoxyphenyl)-1-(1,2,3,4-tetrahydronaphthalen-1-yl)-1H-pyrazolo[3,4-d]pyrimidin-4-amine;
1-(2,3-dihydro-1H-inden-1-yl)-3-(3,4-dimethoxyphenyl)-1H-pyrazolo[3,4-d]pyrimidin-4-amine;
1-(2,3-dihydro-1H-inden-2-yl)-3-(3,4-dimethoxyphenyl)-1H-pyrazolo[3,4-d]pyrimidin-4-amine;
5-(4-amino-1-(2,3-dihydro-1H-inden-1-yl)-1H-pyrazolo[3,4-d]pyrimidin-3-yl)-2-bromophenol;
1-(2,3-dihydro-1H-inden-1-yl)-3-(1H-indol-6-yl)-1H-pyrazolo[3,4-d]pyrimidin-4-amine;
3-(1H-indol-6-yl)-1-(1,2,3,4-tetrahydronaphthalen-1-yl)-1H-pyrazolo[3,4-d]pyrimidin-4-amine;
1-(2,3-dihydro-1H-inden-2-yl)-3-(1H-indol-6-yl)-1H-pyrazolo[3,4-d]pyrimidin-4-amine;
3-(4-amino-1-(2,3-dihydro-1H-inden-2-yl)-1H-pyrazolo[3,4-d]pyrimidin-3-yl)phenol;
3-(4-amino-1-(7-fluoro-1,2,3,4-tetrahydronaphthalen-1-yl)-1H-pyrazolo [3,4-d]pyrimidin-3-yl)-5-fluorophenol;
3-(4-amino-1-(5-fluoro-2,3-dihydro-1H-inden-1-yl)-1H-pyrazolo[3,4-d]pyrimidin-3-yl)-5-fluorophenol;
3-(3-(benzyloxy)-4-(trifluoromethyl)phenyl)-1-(2,3-dihydro-1H-inden-2-yl)-1H-pyrazolo[3,4-d]pyrimidin-4-amine;
5-(4-amino-1-(2,3-dihydro-1H-inden-2-yl)-1H-pyrazolo[3,4-d]pyrimidin-3-yl)-2-methylphenol;
3-(3-aminophenyl)-1-(2,3-dihydro-1H-inden-2-yl)-1H-pyrazolo[3,4-d]pyrimidin-4-amine;
3-(3-amino-4-methylphenyl)-1-(2,3-dihydro-1H-inden-2-yl)-1H-pyrazolo[3,4-d]pyrimidin-4-amine;
3-(1H-benzo[d]imidazol-5-yl)-1-(2,3-dihydro-1H-inden-2-yl)-1H-pyrazolo[3,4-d]pyrimidin-4-amine;
1-(2,3-dihydro-1H-inden-2-yl)-3-morpholino-1H-pyrazolo[3,4-d]pyrimidin-4-amine;
1-(2,3-dihydro-1H-inden-2-yl)-3-(3-methoxy-4-methylphenyl)-1H-pyrazolo[3,4-d]pyrimidin-4-amine;
1-(2,3-dihydro-1H-inden-2-yl)-3-(2-methoxypyridin-4-yl)-1H-pyrazolo[3,4-d]pyrimidin-4-amine;
2-(4-amino-3-(3-fluoro-5-hydroxyphenyl)-1H-pyrazolo[3,4-d]pyrimidin-1-yl)-5-fluoro-2,3-dihydro-1H-inden-1-one;
4-(4-amino-1-(2,3-dihydro-1H-inden-2-yl)-1H-pyrazolo[3,4-d]pyrimidin-3-yl)pyridin-2-ol;
(2-(4-amino-1-(2,3-dihydro-1H-inden-2-yl)-1H-pyrazolo[3,4-d]pyrimidin-3-yl)phenyl)methanol;
(3-(4-amino-1-(2,3-dihydro-1H-inden-2-yl)-1H-pyrazolo[3,4-d]pyrimidin-3-yl)phenyl)methanol;
(4-(4-amino-1-(2,3-dihydro-1H-inden-2-yl)-1H-pyrazolo[3,4-d]pyrimidin-3-yl)phenyl)methanol;
5-(4-amino-1-(2,3-dihydro-1H-inden-2-yl)-1H-pyrazolo[3,4-d]pyrimidin-3-yl)-2-chlorophenol;
3-(4-amino-1-(5-fluoro-2,3-dihydro-1H-inden-2-yl)-1H-pyrazolo[3,4-d]pyrimidin-3-yl)-5-fluorophenol;
(E)-3-(4-amino-1-(2,3-dihydro-1H-inden-2-yl)-1H-pyrazolo[3,4-d]pyrimidin-3-yl)acrylamide;
N-(3-(4-amino-1-(2,3-dihydro-1H-inden-2-yl)-1H-pyrazolo[3,4-d]pyrimidin-3-yl)phenyl)acetamide;
N-(3-(4-amino-1-(2,3-dihydro-1H-inden-2-yl)-1H-pyrazolo[3,4-d]pyrimidin-3-yl)-5-fluorophenyl)acetamide;
1-(2,3-dihydro-1H-inden-2-yl)-3-(5-methoxypyridin-3-yl)-1H-pyrazolo[3,4-d]pyrimidin-4-amine;
7-(2,3-dihydro-1H-inden-2-yl)-5-(3-fluoro-5-methoxyphenyl)-7H-pyrrolo[2,3-d]pyrimidin-4-amine;
3-(4-amino-7-(2,3-dihydro-1H-inden-2-yl)-7H-pyrrolo[2,3-d]pyrimidin-5-yl)-5-fluorophenol;
(E)-3-(4-amino-1-(2,3-dihydro-1H-inden-2-yl)-1H-pyrazolo[3,4-d]pyrimidin-3-yl)prop-2-en-1-ol;
3-(4-amino-1-(2,3-dihydro-1H-inden-2-yl)-1H-pyrazolo[3,4-d]pyrimidin-3-yl)propan-1-ol;
3-(4-amino-3-methoxyphenyl)-1-(2,3-dihydro-1H-inden-2-yl)-1H-pyrazolo[3,4-d]pyrimidin-4-amine;
3-(4-amino-1-(2,3-dihydro-1H-inden-2-yl)-1H-pyrazolo[3,4-d]pyrimidin-3-yl)-5-chlorophenol;
5-(4-amino-1-(2,3-dihydro-1H-inden-2-yl)-1H-pyrazolo[3,4-d]pyrimidin-3-yl)-2-methoxyphenol;
1-(2,3-dihydro-1H-inden-2-yl)-3-(3-methoxy-4-(trifluoromethyl)phenyl)-1H-pyrazolo[3,4-d]pyrimidin-4-amine;
1-(2,3-dihydro-1H-inden-2-yl)-3-(1H-pyrrolo[2,3-b]pyridin-4-yl)-1H-pyrazolo[3,4-d]pyrimidin-4-amine;
1-(2,3-dihydro-1H-inden-2-yl)-3-(4-ethoxy-3-methoxyphenyl)-1H-pyrazolo[3,4-d]pyrimidin-4-amine;
3-(4-amino-1-(2,3-dihydro-1H-inden-2-yl)-1H-pyrazolo[3,4-d]pyrimidin-3-yl)-5-(trifluoromethoxy)phenol;
3-(3-amino-4-methoxyphenyl)-1-(2,3-dihydro-1H-inden-2-yl)-1H-pyrazolo[3,4-d]pyrimidin-4-amine;
1-(2,3-dihydro-1H-inden-2-yl)-3-(3,4-dihydro-2H-benzo[b][1,4]dioxepin-7-yl)-1H-pyrazolo[3,4-d]pyrimidin-4-amine;
3-(2,2-difluorobenzo[d][1,3]dioxol-5-yl)-1-(2,3-dihydro-1H-inden-2-yl)-1H-pyrazolo[3,4-d]pyrimidin-4-amine;
1-(2,3-dihydro-1H-inden-2-yl)-3-(3-ethoxy-4-(trifluoromethoxy)phenyl)-1H-pyrazolo[3,4-d]pyrimidin-4-amine;
3-(3-chloro-4-ethoxy-5-methoxyphenyl)-1-(2,3-dihydro-1H-inden-2-yl)-1H-pyrazolo[3,4-d]pyrimidin-4-amine;
1-(2,3-dihydro-1H-inden-2-yl)-3-(1H-indol-4-yl)-1H-pyrazolo[3,4-d]pyrimidin-4-amine;
3-(3-chloro-5-ethoxy-4-isopropoxyphenyl)-1-(2,3-dihydro-1H-inden-2-yl)-1H-pyrazolo[3,4-d]pyrimidin-4-amine;
3-(3-chloro-4,5-dimethoxyphenyl)-1-(2,3-dihydro-1H-inden-2-yl)-1H-pyrazolo[3,4-d]pyrimidin-4-amine;
5-(4-amino-1-(2,3-dihydro-1H-inden-2-yl)-1H-pyrazolo[3,4-d]pyrimidin-3-yl)-2,3-difluorophenol;
1-(2,3-dihydro-1H-inden-2-yl)-3-(2-methyl-1H-benzo[d]imidazol-6-yl)-1H-pyrazolo[3,4-d]pyrimidin-4-amine;
1-(2,3-dihydro-1H-inden-2-yl)-3-(2,3,4-trimethoxyphenyl)-1H-pyrazolo[3,4-d]pyrimidin-4-amine;
1-(2,3-dihydro-1H-inden-2-yl)-3-(8-fluoro-3,4-dihydro-2H-benzo[b][1,4]oxazin-6-yl)-1H-pyrazolo[3,4-d]pyrimidin-4-amine;
4-(4-amino-1-(2,3-dihydro-1H-inden-2-yl)-1H-pyrazolo[3,4-d]pyrimidin-3-yl)-2-methoxyphenol;
1-(2,3-dihydro-1H-inden-2-yl)-3-(3,4,5-trifluorophenyl)-1H-pyrazolo[3,4-d]pyrimidin-4-amine;
3-(3-amino-4-fluorophenyl)-1-(2,3-dihydro-1H-inden-2-yl)-1H-pyrazolo[3,4-d]pyrimidin-4-amine;
1-(2,3-dihydro-1H-inden-2-yl)-3-(3-fluorophenyl)-1H-pyrazolo[3,4-d]pyrimidin-4-amine;
3-(3,4-difluorophenyl)-1-(2,3-dihydro-1H-inden-2-yl)-1H-pyrazolo[3,4-d]pyrimidin-4-amine;
3-(3-butoxy-4-methoxyphenyl)-1-(2,3-dihydro-1H-inden-2-yl)-1H-pyrazolo[3,4-d]pyrimidin-4-amine;
3-(3,4-difluoro-5-methoxyphenyl)-1-(2,3-dihydro-1H-inden-2-yl)-1H-pyrazolo[3,4-d]pyrimidin-4-amine;
1-(2,3-dihydro-1H-inden-2-yl)-3-(4-fluoro-3-methoxy-5-(trifluoromethyl)phenyl)-1H-pyrazolo[3,4-d]pyrimidin-4-amine;
5-(4-amino-7-(2,3-dihydro-1H-inden-2-yl)-7H-pyrrolo[2,3-d]pyrimidin-5-yl)-2-chlorophenol;
4-(4-amino-1-(2,3-dihydro-1H-inden-2-yl)-1H-pyrazolo[3,4-d]pyrimidin-3-yl)-2-hydroxybenzonitrile;
3-(2,4-dichloro-3-methoxyphenyl)-1-(2,3-dihydro-1H-inden-2-yl)-1H-pyrazolo[3,4-d]pyrimidin-4-amine;
3-(6-aminopyridin-3-yl)-1-(2,3-dihydro-1H-inden-2-yl)-1H-pyrazolo[3,4-d]pyrimidin-4-amine;
5-(4-amino-1-(2,3-dihydro-1H-inden-2-yl)-1H-pyrazolo[3,4-d]pyrimidin-3-yl)-2-fluorobenzonitrile;
1-(2,3-dihydro-1H-inden-2-yl)-3-(2-methyl-3-(methylamino)phenyl)-1H-pyrazolo[3,4-d]pyrimidin-4-amine;
1-(2,3-dihydro-1H-inden-2-yl)-3-(3,4-dihydro-2H-benzo[b][1,4]oxazin-6-yl)-1H-pyrazolo[3,4-d]pyrimidin-4-amine;
3-(4-amino-1-(2,3-dihydro-1H-inden-2-yl)-1H-pyrazolo[3,4-d]pyrimidin-3-yl)-6-chloro-2-fluorophenol;
5-(4-amino-7-(2,3-dihydro-1H-inden-2-yl)-7H-pyrrolo[2,3-d]pyrimidin-5-yl)-2,3-difluorophenol;
5-(4-amino-1-(2,3-dihydro-1H-inden-2-yl)-1H-pyrazolo[3,4-d]pyrimidin-3-yl)-2-fluoro-3-(trifluoromethyl)phenol;
5-(4-amino-1-(2,3-dihydro-1H-inden-2-yl)-1H-pyrazolo[3,4-d]pyrimidin-3-yl)-2,4-dichlorophenol;
3-(5-amino-6-chloropyridin-3-yl)-1-(2,3-dihydro-1H-inden-2-yl)-1H-pyrazolo[3,4-d]pyrimidin-4-amine;
5-(4-amino-1-(2,3-dihydro-1H-inden-2-yl)-1H-pyrazolo[3,4-d]pyrimidin-3-yl)-2,3-dimethoxybenzonitrile;
2-(4-amino-3-(4-chloro-3-hydroxyphenyl)-1H-pyrazolo[3,4-d]pyrimidin-1-yl)-2,3-dihydro-1H-inden-1-ol;
5-(4-amino-7-(2,3-dihydro-1H-inden-2-yl)-7H-pyrrolo[2,3-d]pyrimidin-5-yl)-2,3-dichlorophenol;
5-(4-amino-1-(2,3-dihydro-1H-inden-2-yl)-1H-pyrazolo[3,4-d]pyrimidin-3-yl)-1H-benzo[d]imidazol-2-ol;
3-(3-amino-4,5-difluorophenyl)-1-(2,3-dihydro-1H-inden-2-yl)-1H-pyrazolo[3,4-d]pyrimidin-4-amine;
5-(4-amino-7-(2,3-dihydro-1H-inden-2-yl)-7H-pyrrolo[2,3-d]pyrimidin-5-yl)-3-chloro-2-fluorophenol;
4-(4-amino-1-(2,3-dihydro-1H-inden-2-yl)-1H-pyrazolo[3,4-d]pyrimidin-3-yl)-2-hydroxybenzamide;
5-(4-amino-1-(2,3-dihydro-1H-inden-2-yl)-1H-pyrazolo[4,3-c]pyridin-3-yl)-2-chlorophenol;
5-(3,4-difluoro-5-(methylamino)phenyl)-7-(2,3-dihydro-1H-inden-2-yl)-7H-pyrrolo[2,3-d]pyrimidin-4-amine;
5-(4-amino-7-(1,2,3,4-tetrahydronaphthalen-2-yl)-7H-pyrrolo[2,3-d]pyrimidin-5-yl)-2,3-difluorophenol;
5-(4-amino-7-(2,3-dihydro-1H-inden-2-yl)-7H-pyrrolo[2,3-d]pyrimidin-5-yl)-2-fluoro-3-hydroxybenzonitrile;
5-(4-amino-7-(2,3-dihydro-1H-inden-2-yl)-7H-pyrrolo[2,3-d]pyrimidin-5-yl)-2-fluoro-3-hydroxybenzamide;
5-(4-amino-1-(2,3-dihydro-1H-inden-2-yl)-1H-pyrazolo[3,4-d]pyrimidin-3-yl)-3-chloro-2-fluorophenol;
5-(4-amino-1-(2,3-dihydro-1H-inden-2-yl)-1H-pyrazolo[3,4-d]pyrimidin-3-yl)-2,3-dichlorophenol;
5-(4-amino-1-(1,2,3,4-tetrahydronaphthalen-2-yl)-1H-pyrazolo[3,4-d]pyrimidin-3-yl)-2,3-difluorophenol;
5-(4-amino-1-(2,3-dihydro-1H-inden-2-yl)-1H-pyrazolo[3,4-d]pyrimidin-3-yl)-2-fluoro-3-hydroxybenzonitrile;
1-(5-fluoro-2,3-dihydro-1H-inden-2-yl)-3-(8-fluoro-3,4-dihydro-2H-benzo[b][1,4]oxazin-6-yl)-1H-pyrazolo[3,4-d]pyrimidin-4-amine;
3-(3-amino-4,5-difluorophenyl)-1-(1,2,3,4-tetrahydronaphthalen-2-yl)-1H-pyrazolo[3,4-d]pyrimidin-4-amine;
5-(4-amino-1-(1,2,3,4-tetrahydronaphthalen-2-yl)-1H-pyrazolo[3,4-d]pyrimidin-3-yl)-2-fluoro-3-(trifluoromethyl)phenol;
3-(8-fluoro-3,4-dihydro-2H-benzo[b][1,4]oxazin-6-yl)-1-(1,2,3,4-tetrahydronaphthalen-2-yl)-1H-pyrazolo[3,4-d]pyrimidin-4-amine;
5-(4-amino-7-(2,3-dihydro-1H-inden-2-yl)pyrrolo[1,2-f][1,2,4]triazin-5-yl)-2,3-difluorophenol;
5-(4-amino-1-(6,7-dihydro-5H-cyclopenta[b]pyridin-5-yl)-1H-pyrazolo[3,4-d]pyrimidin-3-yl)-2,3-difluorophenol;
2-(4-amino-3-(3,4-difluoro-5-hydroxyphenyl)-1H-pyrazolo[3,4-d]pyrimidin-1-yl)-5-fluoro-2,3-dihydro-1H-inden-1-one;
5-(4-amino-1-(5-fluoro-2,3-dihydro-1H-inden-2-yl)-1H-pyrazolo[3,4-d]pyrimidin-3-yl)-2,3-difluorophenol;
6-(4-amino-1-(2,3-dihydro-1H-inden-2-yl)-1H-pyrazolo[3,4-d]pyrimidin-3-yl)-8-fluoro-2H-benzo[b][1,4]oxazin-3(4H)-one;
5-(4-amino-1-(1,2,3,4-tetrahydronaphthalen-2-yl)-1H-pyrazolo[3,4-d]pyrimidin-3-yl)-2-fluoro-3-hydroxybenzonitrile;
5-(4-amino-1-(1,2,3,4-tetrahydronaphthalen-2-yl)-1H-pyrazolo[3,4-d]pyrimidin-3-yl)-2-fluoro-3-hydroxybenzamide;
5-(4-amino-1-(2,3-dihydro-1H-inden-2-yl)-1H-pyrazolo[3,4-d]pyrimidin-3-yl)-2-fluoro-3-hydroxybenzamide;
3-(3-(aminomethyl)phenyl)-1-(2,3-dihydro-1H-inden-2-yl)-1H-pyrazolo[3,4-d]pyrimidin-4-amine;
5-(4-amino-6-chloro-7-(2,3-dihydro-1H-inden-2-yl)-7H-pyrrolo[2,3-d]pyrimidin-5-yl)-2,3-difluorophenol;
4-amino-5-(3,4-difluoro-5-hydroxyphenyl)-7-(2,3-dihydro-1H-inden-2-yl)-7H-pyrrolo[2,3-d]pyrimidine-6-carbonitrile;
5-(4-amino-7-(2,3-dihydro-1H-inden-2-yl)-6-fluoro-7H-pyrrolo[2,3-d]pyrimidin-5-yl)-2,3-difluorophenol;
5-(4-amino-7-(2,3-dihydro-1H-inden-2-yl)-5H-pyrrolo[3,2-d]pyrimidin-5-yl)-2,3-difluorophenol;
3-(4-(aminomethyl)phenyl)-1-(2,3-dihydro-1H-inden-2-yl)-1H-pyrazolo[3,4-d]pyrimidin-4-amine;
5-(4-amino-1-(5-amino-2,3-dihydro-1H-inden-2-yl)-1H-pyrazolo[3,4-d]pyrimidin-3-yl)-2,3-difluorophenol; and
5-(8-amino-3-(2,3-dihydro-1H-inden-2-yl)imidazo[1,5-a]pyrazin-1-yl)-2,3-difluorophenol.

27. A compound selected from the group consisting of:
5-(4-amino-1-(1,2,3,4-tetrahydronaphthalen-2-yl)-1H-pyrazolo[3,4-d]pyrimidin-3-yl)-2-chlorophenol;
3-(4-ethoxy-3-methoxyphenyl)-1-(1,2,3,4-tetrahydronaphthalen-2-yl)-1H-pyrazolo[3,4-d]pyrimidin-4-amine;
1-(2,3-dihydro-1H-inden-2-yl)-3-(5-fluoro-6-phenoxy-1H-indol-2-yl)-1H-pyrazolo[3,4-d]pyrimidin-4-amine;
3-(4-amino-1-(2,3-dihydro-1H-inden-1-yl)-1H-pyrazolo[3,4-d]pyrimidin-3-yl)benzamide;
5-(4-amino-1-(2,3-dihydro-1H-inden-1-yl)-1H-pyrazolo[3,4-d]pyrimidin-3-yl)-2-chlorophenol;
5-(4-amino-1-(2,3-dihydro-1H-inden-1-yl)-1H-pyrazolo[3,4-d]pyrimidin-3-yl)-2,3-difluorophenol;
5-(4-amino-1-(2,3-dihydro-1H-inden-1-yl)-1H-pyrazolo[3,4-d]pyrimidin-3-yl)-2-fluoro-3-(trifluoromethyl)phenol;
1-(2,3-dihydro-1H-inden-1-yl)-3-(4-ethoxy-3-methoxyphenyl)-1H-pyrazolo[3,4-d]pyrimidin-4-amine;
4-(4-amino-1-(2,3-dihydro-1H-inden-1-yl)-1H-pyrazolo[3,4-d]pyrimidin-3-yl)benzene-1,2-diol;
4-(4-amino-1-(2,3-dihydro-1H-inden-1-yl)-1H-pyrazolo[3,4-d]pyrimidin-3-yl)phenol;
1-(2,3-dihydro-1H-inden-1-yl)-3-(1H-pyrrolo[2,3-b]pyridin-5-yl)-1H-pyrazolo[3,4-d]pyrimidin-4-amine;
1-(2,3-dihydro-1H-inden-1-yl)-3-(5-fluoro-6-methoxypyridin-3-yl)-1H-pyrazolo[3,4-d]pyrimidin-4-amine;
4-(4-amino-1-(2,3-dihydro-1H-inden-2-yl)-1H-pyrazolo[3,4-d]pyrimidin-3-yl)benzamide;
3-(4-amino-1-(2,3-dihydro-1H-inden-2-yl)-1H-pyrazolo[3,4-d]pyrimidin-3-yl)benzamide;
5-(4-amino-1-(1,2,3,4-tetrahydronaphthalen-1-yl)-1H-pyrazolo[3,4-d]pyrimidin-3-yl)-2-chlorophenol;
5-(4-amino-1-(1,2,3,4-tetrahydronaphthalen-1-yl)-1H-pyrazolo[3,4-d]pyrimidin-3-yl)-2-bromophenol;
5-(4-amino-1-(1,2,3,4-tetrahydronaphthalen-1-yl)-1H-pyrazolo[3,4-d]pyrimidin-3-yl)-2,3-difluorophenol;
3-(4-ethoxy-3-methoxyphenyl)-1-(1,2,3,4-tetrahydronaphthalen-1-yl)-1H-pyrazolo[3,4-d]pyrimidin-4-amine;
4-(4-amino-1-(1,2,3,4-tetrahydronaphthalen-1-yl)-1H-pyrazolo[3,4-d]pyrimidin-3-yl)benzene-1,2-diol;
4-(4-amino-1-(1,2,3,4-tetrahydronaphthalen-1-yl)-1H-pyrazolo[3,4-d]pyrimidin-3-yl)phenol;
3-(1H-pyrrolo[2,3-b]pyridin-5-yl)-1-(1,2,3,4-tetrahydronaphthalen-1-yl)-1H-pyrazolo[3,4-d]pyrimidin-4-amine;
3-(5-fluoro-6-methoxypyridin-3-yl)-1-(1,2,3,4-tetrahydronaphthalen-1-yl)-1H-pyrazolo[3,4-d]pyrimidin-4-amine;
1-(3-(4-amino-3-(3-fluoro-5-hydroxyphenyl)-1H-pyrazolo[3,4-d]pyrimidin-1-yl)-3,4-dihydroquinolin-1(2H)-yl)prop-2-en-1-one;
1-(3-(4-amino-3-(4-fluoro-3-hydroxyphenyl)-1H-pyrazolo[3,4-d]pyrimidin-1-yl)-3,4-dihydroquinolin-1(2H)-yl)prop-2-en-1-one;
1-(3-(4-amino-3-(4-chloro-3-hydroxyphenyl)-1H-pyrazolo[3,4-d]pyrimidin-1-yl)-3,4-dihydroquinolin-1(2H)-yl)prop-2-en-1-one;
1-(3-(4-amino-3-(4-bromo-3-hydroxyphenyl)-1H-pyrazolo[3,4-d]pyrimidin-1-yl)-3,4-dihydroquinolin-1(2H)-yl)prop-2-en-1-one;
1-(3-(4-amino-3-(3,4-dimethoxyphenyl)-1H-pyrazolo[3,4-d]pyrimidin-1-yl)-3,4-dihydroquinolin-1(2H)-yl)prop-2-en-1-one;
1-(3-(4-amino-3-(4-ethoxy-3-methoxyphenyl)-1H-pyrazolo[3,4-d]pyrimidin-1-yl)-3,4-dihydroquinolin-1(2H)-yl)prop-2-en-1-one;
1-(3-(4-amino-3-(3,4-dihydroxyphenyl)-1H-pyrazolo[3,4-d]pyrimidin-1-yl)-3,4-dihydroquinolin-1(2H)-yl)prop-2-en-1-one;
1-(3-(4-amino-3-(4-hydroxyphenyl)-1H-pyrazolo[3,4-d]pyrimidin-1-yl)-3,4-dihydroquinolin-1(2H)-yl)prop-2-en-1-one;
1-(3-(4-amino-3-(1H-pyrrolo[2,3-b]pyridin-5-yl)-1H-pyrazolo[3,4-d]pyrimidin-1-yl)-3,4-dihydroquinolin-1(2H)-yl)prop-2-en-1-one;
1-(3-(4-amino-3-(5-fluoro-6-methoxypyridin-3-yl)-1H-pyrazolo[3,4-d]pyrimidin-1-yl)-3,4-dihydroquinolin-1(2H)-yl)prop-2-en-1-one;
1-(3-(4-amino-3-(3-fluoro-5-hydroxyphenyl)-1H-pyrazolo[3,4-d]pyrimidin-1-yl)indolin-1-yl)prop-2-en-1-one;
1-(3-(4-amino-3-(4-fluoro-3-hydroxyphenyl)-1H-pyrazolo[3,4-d]pyrimidin-1-yl)indolin-1-yl)prop-2-en-1-one;
1-(3-(4-amino-3-(4-chloro-3-hydroxyphenyl)-1H-pyrazolo[3,4-d]pyrimidin-1-yl)indolin-1-yl)prop-2-en-1-one;
1-(3-(4-amino-3-(4-bromo-3-hydroxyphenyl)-1H-pyrazolo[3,4-d]pyrimidin-1-yl)indolin-1-yl)prop-2-en-1-one;
1-(3-(4-amino-3-(3,4-dimethoxyphenyl)-1H-pyrazolo[3,4-d]pyrimidin-1-yl)indolin-1-yl)prop-2-en-1-one;
1-(3-(4-amino-3-(4-ethoxy-3-methoxyphenyl)-1H-pyrazolo[3,4-d]pyrimidin-1-yl)indolin-1-yl)prop-2-en-1-one;
1-(3-(4-amino-3-(3,4-dihydroxyphenyl)-1H-pyrazolo[3,4-d]pyrimidin-1-yl)indolin-1-yl)prop-2-en-1-one;
1-(3-(4-amino-3-(4-hydroxyphenyl)-1H-pyrazolo[3,4-d]pyrimidin-1-yl)indolin-1-yl)prop-2-en-1-one;
1-(3-(4-amino-3-(1H-pyrrolo[2,3-b]pyridin-5-yl)-1H-pyrazolo[3,4-d]pyrimidin-1-yl)indolin-1-yl)prop-2-en-1-one;
1-(3-(4-amino-3-(5-fluoro-6-methoxypyridin-3-yl)-1H-pyrazolo[3,4-d]pyrimidin-1-yl)indolin-1-yl)prop-2-en-1-one;
1-(4-(4-amino-3-(3-fluoro-5-hydroxyphenyl)-1H-pyrazolo[3,4-d]pyrimidin-1-yl)-3,4-dihydroisoquinolin-2(1H)-yl)prop-2-en-1-one;
1-(4-(4-amino-3-(4-fluoro-3-hydroxyphenyl)-1H-pyrazolo[3,4-d]pyrimidin-1-yl)-3,4-dihydroisoquinolin-2(1H)-yl)prop-2-en-1-one;
1-(4-(4-amino-3-(4-chloro-3-hydroxyphenyl)-1H-pyrazolo[3,4-d]pyrimidin-1-yl)-3,4-dihydroisoquinolin-2(1H)-yl)prop-2-en-1-one;
1-(4-(4-amino-3-(4-bromo-3-hydroxyphenyl)-1H-pyrazolo[3,4-d]pyrimidin-1-yl)-3,4-dihydroisoquinolin-2(1H)-yl)prop-2-en-1-one;
1-(4-(4-amino-3-(3,4-dimethoxyphenyl)-1H-pyrazolo[3,4-d]pyrimidin-1-yl)-3,4-dihydroisoquinolin-2(1H)-yl)prop-2-en-1-one;
1-(4-(4-amino-3-(4-ethoxy-3-methoxyphenyl)-1H-pyrazolo[3,4-d]pyrimidin-1-yl)-3,4-dihydroisoquinolin-2(1H)-yl)prop-2-en-1-one;
1-(4-(4-amino-3-(3,4-dihydroxyphenyl)-1H-pyrazolo[3,4-d]pyrimidin-1-yl)-3,4-dihydroisoquinolin-2(1H)-yl)prop-2-en-1-one;
1-(4-(4-amino-3-(4-hydroxyphenyl)-1H-pyrazolo[3,4-d]pyrimidin-1-yl)-3,4-dihydroisoquinolin-2(1H)-yl)prop-2-en-1-one;
1-(4-(4-amino-3-(1H-pyrrolo[2,3-b]pyridin-5-yl)-1H-pyrazolo[3,4-d]pyrimidin-1-yl)-3,4-dihydroisoquinolin-2(1H)-yl)prop-2-en-1-one;
1-(4-(4-amino-3-(5-fluoro-6-methoxypyridin-3-yl)-1H-pyrazolo[3,4-d]pyrimidin-1-yl)-3,4-dihydroisoquinolin-2(1H)-yl)prop-2-en-1-one;
3-(4-amino-1-(1-(vinylsulfonyl)-1,2,3,4-tetrahydroquinolin-3-yl)-1H-pyrazolo[3,4-d]pyrimidin-3-yl)-5-fluorophenol;
1-(3-(4-amino-3-(5-fluoro-6-phenoxy-1H-indol-2-yl)-1H-pyrazolo[3,4-d]pyrimidin-1-yl)-3,4-dihydroquinolin-1(2H)-yl)prop-2-en-1-one;
1-(3-(4-amino-3-(6-fluoro-5-phenoxy-1H-indol-2-yl)-1H-pyrazolo[3,4-d]pyrimidin-1-yl)-3,4-dihydroquinolin-1(2H)-yl)prop-2-en-1-one;
3-(5-fluoro-6-phenoxy-1H-indol-2-yl)-1-(1,2,3,4-tetrahydronaphthalen-2-yl)-1H-pyrazolo[3,4-d]pyrimidin-4-amine;
3-(6-fluoro-5-phenoxy-1H-indol-2-yl)-1-(1,2,3,4-tetrahydronaphthalen-2-yl)-1H-pyrazolo[3,4-d]pyrimidin-4-amine;
1-(2,3-dihydro-1H-inden-2-yl)-3-(6-fluoro-5-phenoxy-1H-indol-1-yl)-1H-pyrazolo[3,4-d]pyrimidin-4-amine;
2-(3-(4-amino-3-(3-fluoro-5-hydroxyphenyl)-1H-pyrazolo[3,4-d]pyrimidin-1-yl)-2-oxo-3,4-dihydroquinolin-1(2H)-yl)acetonitrile;
3-(3-(4-amino-3-(3-fluoro-5-hydroxyphenyl)-1H-pyrazolo[3,4-d]pyrimidin-1-yl)-2-oxo-3,4-dihydroquinolin-1(2H)-yl)propanenitrile;
5-(4-amino-1-(1,2,3,4-tetrahydronaphthalen-2-yl)-1H-pyrazolo[3,4-d]pyrimidin-3-yl)-2-methylphenol;
5-(4-amino-1-(1,2,3,4-tetrahydronaphthalen-2-yl)-1H-pyrazolo[3,4-d]pyrimidin-3-yl)-2-(trifluoromethyl)phenol;
(3-(4-amino-1-(1,2,3,4-tetrahydronaphthalen-2-yl)-1H-pyrazolo[3,4-d]pyrimidin-3-yl)phenyl)methanol;
(4-(4-amino-1-(1,2,3,4-tetrahydronaphthalen-2-yl)-1H-pyrazolo[3,4-d]pyrimidin-3-yl)phenyl)methanol;
(2-(4-amino-1-(1,2,3,4-tetrahydronaphthalen-2-yl)-1H-pyrazolo[3,4-d]pyrimidin-3-yl)phenyl)methanol;
4-(4-amino-1-(1,2,3,4-tetrahydronaphthalen-1-yl)-1H-pyrazolo[3,4-d]pyrimidin-3-yl)benzamide;
3-(4-amino-1-(1,2,3,4-tetrahydronaphthalen-1-yl)-1H-pyrazolo[3,4-d]pyrimidin-3-yl)benzamide;
(5-(4-amino-1-(1,2,3,4-tetrahydronaphthalen-2-yl)-1H-pyrazolo[3,4-d]pyrimidin-3-yl)-2-chlorophenyl)methanol;
4-{[4-amino-1-(1,2,3,4-tetrahydronaphthalen-2-yl)pyrazolo[3,4-d]pyrimidin-3-yl]methyl}-1λ⁶,2-thiazole-1,1-dione;
4-[4-amino-1-(1,2,3,4-tetrahydronaphthalen-2-yl)pyrazolo[3,4-d]pyrimidin-3-yl]-1λ⁶,2-thiazole-1,1-dione;
1-(4-amino-1-(1,2,3,4-tetrahydronaphthalen-2-yl)-1H-pyrazolo[3,4-d]pyrimidin-3-yl)-2-thioxoimidazolidin-4-one;
1-(4-amino-1-(1,2,3,4-tetrahydronaphthalen-2-yl)-1H-pyrazolo[3,4-d]pyrimidin-3-yl)imidazolidine-2,4-dione;
3-(pyrimidin-4-yl)-1-(1,2,3,4-tetrahydronaphthalen-2-yl)-1H-pyrazolo[3,4-d]pyrimidin-4-amine;
3-(pyridin-4-yl)-1-(1,2,3,4-tetrahydronaphthalen-2-yl)-1H-pyrazolo[3,4-d]pyrimidin-4-amine;
3-((1,2,4-oxadiazol-3-yl)methyl)-1-(1,2,3,4-tetrahydronaphthalen-2-yl)-1H-pyrazolo[3,4-d]pyrimidin-4-amine;
3-(1,2,4-oxadiazol-3-yl)-1-(1,2,3,4-tetrahydronaphthalen-2-yl)-1H-pyrazolo[3,4-d]pyrimidin-4-amine;
1-(4-(4-amino-1-(1,2,3,4-tetrahydronaphthalen-2-yl)-1H-pyrazolo[3,4-d]pyrimidin-3-yl)phenyl)ethanone;
1-(3-(4-amino-1-(1,2,3,4-tetrahydronaphthalen-2-yl)-1H-pyrazolo[3,4-d]pyrimidin-3-yl)phenyl)ethanone;
3-(tetrahydro-2H-pyran-4-yl)-1-(1,2,3,4-tetrahydronaphthalen-2-yl)-1H-pyrazolo[3,4-d]pyrimidin-4-amine;
4-(4-amino-1-(1,2,3,4-tetrahydronaphthalen-2-yl)-1H-pyrazolo[3,4-d]pyrimidin-3-yl)cyclohexanone;
3-(4-amino-1-(8-fluoro-1,2,3,4-tetrahydronaphthalen-2-yl)-1H-pyrazolo[3,4-d]pyrimidin-3-yl)-5-fluorophenol;
3-(4-amino-1-(7-fluoro-1,2,3,4-tetrahydronaphthalen-2-yl)-1H-pyrazolo[3,4-d]pyrimidin-3-yl)-5-fluorophenol;
3-(4-amino-1-(6-fluoro-1,2,3,4-tetrahydronaphthalen-2-yl)-1H-pyrazolo[3,4-d]pyrimidin-3-yl)-5-fluorophenol;
3-(4-amino-1-(5-fluoro-1,2,3,4-tetrahydronaphthalen-2-yl)-1H-pyrazolo[3,4-d]pyrimidin-3-yl)-5-fluorophenol;
5-(4-amino-1-(1,2,3,4-tetrahydronaphthalen-2-yl)-1H-pyrazolo[3,4-d]pyrimidin-3-yl)-2-isopropoxyphenol;
5-(4-amino-1-(1,2,3,4-tetrahydronaphthalen-2-yl)-1H-pyrazolo[3,4-d]pyrimidin-3-yl)-2-methoxyphenol;
3-(4-methoxy-3-(methylamino)phenyl)-1-(1,2,3,4-tetrahydronaphthalen-2-yl)-1H-pyrazolo[3,4-d]pyrimidin-4-amine;
3-(4-fluoro-3-(methylamino)phenyl)-1-(1,2,3,4-tetrahydronaphthalen-2-yl)-1H-pyrazolo[3,4-d]pyrimidin-4-amine;
4-(4-amino-1-(1,2,3,4-tetrahydronaphthalen-2-yl)-1H-pyrazolo[3,4-d]pyrimidin-3-yl)-2-methoxyphenol;
4-(4-amino-1-(1,2,3,4-tetrahydronaphthalen-2-yl)-1H-pyrazolo[3,4-d]pyrimidin-3-yl)-2-(trifluoromethyl)phenol;
5-(4-amino-1-(1,2,3,4-tetrahydronaphthalen-2-yl)-1H-pyrazolo[3,4-d]pyrimidin-3-yl)-2-hydroxybenzonitrile;
4-(4-amino-1-(2,3-dihydro-1H-inden-1-yl)-1H-pyrazolo[3,4-d]pyrimidin-3-yl)benzamide;
5-(4-amino-1-(2,3-dihydro-1H-inden-2-yl)-1H-pyrazolo[3,4-d]pyrimidin-3-yl)-2-isopropoxyphenol;
5-(4-amino-1-(2,3-dihydro-1H-inden-2-yl)-1H-pyrazolo[3,4-d]pyrimidin-3-yl)-2-chloro-3-fluorophenol;
5-(4-amino-1-(2,3-dihydro-1H-inden-2-yl)-1H-pyrazolo[3,4-d]pyrimidin-3-yl)-2-chloro-3-hydroxybenzonitrile;
4-(4-amino-1-(2,3-dihydro-1H-inden-2-yl)-1H-pyrazolo[3,4-d]pyrimidin-3-yl)benzene-1,2-diol;
1-(2,3-dihydro-1H-inden-2-yl)-3-(4-methoxy-3-(methylamino)phenyl)-1H-pyrazolo[3,4-d]pyrimidin-4-amine;
1-(2,3-dihydro-1H-inden-2-yl)-3-(4-fluoro-3-(methylamino)phenyl)-1H-pyrazolo[3,4-d]pyrimidin-4-amine;
4-(4-amino-1-(2,3-dihydro-1H-inden-2-yl)-1H-pyrazolo[3,4-d]pyrimidin-3-yl)phenol;
5-(3,4-difluoro-5-methoxyphenyl)-7-(2,3-dihydro-1H-inden-2-yl)-6-fluoro-7H-pyrrolo[2,3-d]pyrimidin-4-amine;
4-(4-amino-1-(2,3-dihydro-1H-inden-2-yl)-1H-pyrazolo[3,4-d]pyrimidin-3-yl)-2-(trifluoromethyl)phenol;
5-(4-amino-1-(2,3-dihydro-1H-inden-2-yl)-1H-pyrazolo[3,4-d]pyrimidin-3-yl)-2-hydroxybenzonitrile;
1-(2,3-dihydro-1H-inden-2-yl)-3-(5-fluoro-6-phenoxy-1H-indol-1-yl)-1H-pyrazolo[3,4-d]pyrimidin-4-amine;
3-(6-fluoro-5-phenoxy-1H-indol-1-yl)-1-(1,2,3,4-tetrahydronaphthalen-2-yl)-1H-pyrazolo[3,4-d]pyrimidin-4-amine;
1-(3-(4-amino-3-(5-fluoro-6-phenoxy-1H-indol-1-yl)-1H-pyrazolo[3,4-d]pyrimidin-1-yl)-3,4-dihydroquinolin-1(2H)-yl)prop-2-en-1-one;
1-(3-(4-amino-3-(6-fluoro-5-phenoxy-1H-indol-1-yl)-1H-pyrazolo[3,4-d]pyrimidin-1-yl)-3,4-dihydroquinolin-1(2H)-yl)prop-2-en-1-one;
3-(5-fluoro-6-phenoxy-1H-indol-1-yl)-1-(1,2,3,4-tetrahydronaphthalen-2-yl)-1H-pyrazolo[3,4-d]pyrimidin-4-amine;
3-(5-(allyloxy)-6-fluoro-1H-indol-2-yl)-1-(2,3-dihydro-1H-inden-2-yl)-1H-pyrazolo[3,4-d]pyrimidin-4-amine;
1-(3-(3-(6-(allyloxy)-5-fluoro-1H-indol-2-yl)-4-amino-1H-pyrazolo[3,4-d]pyrimidin-1-yl)-3,4-dihydroquinolin-1(2H)-yl)prop-2-en-1-one;
1-(3-(3-(5-(allyloxy)-6-fluoro-1H-indol-2-yl)-4-amino-1H-pyrazolo[3,4-d]pyrimidin-1-yl)-3,4-dihydroquinolin-1(2H)-yl)prop-2-en-1-one;
3-(6-(allyloxy)-5-fluoro-1H-indol-2-yl)-1-(2,3-dihydro-1H-inden-2-yl)-1H-pyrazolo[3,4-d]pyrimidin-4-amine;
3-(5-(allyloxy)-6-fluoro-1H-indol-2-yl)-1-(1,2,3,4-tetrahydronaphthalen-2-yl)-1H-pyrazolo[3,4-d]pyrimidin-4-amine;
3-(6-(allyloxy)-5-fluoro-1H-indol-2-yl)-1-(1,2,3,4-tetrahydronaphthalen-2-yl)-1H-pyrazolo[3,4-d]pyrimidin-4-amine;
5-(4-amino-1-(8-fluoro-1,2,3,4-tetrahydronaphthalen-2-yl)-1H-pyrazolo[3,4-d]pyrimidin-3-yl)-2-fluorophenol;
5-(4-amino-1-(7-fluoro-1,2,3,4-tetrahydronaphthalen-2-yl)-1H-pyrazolo[3,4-d]pyrimidin-3-yl)-2-fluorophenol;
4-(4-amino-1-(8-fluoro-1,2,3,4-tetrahydronaphthalen-2-yl)-1H-pyrazolo[3,4-d]pyrimidin-3-yl)phenol;
4-(4-amino-1-(7-fluoro-1,2,3,4-tetrahydronaphthalen-2-yl)-1H-pyrazolo[3,4-d]pyrimidin-3-yl)phenol;
5-(4-amino-7-(1,2,3,4-tetrahydronaphthalen-2-yl)-7H-pyrrolo[2,3-d]pyrimidin-5-yl)-2-fluorophenol;
5-(4-amino-6-fluoro-7-(1,2,3,4-tetrahydronaphthalen-2-yl)-7H-pyrrolo[2,3-d]pyrimidin-5-yl)-2-fluorophenol;
5-(4-amino-1-(1,2,3,4-tetrahydronaphthalen-2-yl)-1H-pyrazolo[4,3-c]pyridin-3-yl)-2-fluorophenol;
5-(4-amino-7-(1,2,3,4-tetrahydronaphthalen-2-yl)-5H-pyrrolo[3,2-d]pyrimidin-5-yl)-2-fluorophenol;
5-(7-amino-3-(1,2,3,4-tetrahydronaphthalen-2-yl)-1H-pyrazolo[4,3-d]pyrimidin-1-yl)-2-fluorophenol;
4-(4-amino-7-(1,2,3,4-tetrahydronaphthalen-2-yl)-7H-pyrrolo[2,3-d]pyrimidin-5-yl)phenol;
4-(4-amino-1-(1,2,3,4-tetrahydronaphthalen-2-yl)-1H-pyrazolo[4,3-c]pyridin-3-yl)phenol;
4-(4-amino-7-(1,2,3,4-tetrahydronaphthalen-2-yl)-5H-pyrrolo[3,2-d]pyrimidin-5-yl)phenol;
4-(7-amino-3-(1,2,3,4-tetrahydronaphthalen-2-yl)-1H-pyrazolo[4,3-d]pyrimidin-1-yl)phenol;
5-(3,4-difluoro-5-methoxyphenyl)-7-(2,3-dihydro-1H-inden-2-yl)-7H-pyrrolo[2,3-d]pyrimidin-4-amine;
3-(4-amino-1-(2,3-dihydro-1H-inden-2-yl)-1H-pyrazolo[4,3-c]pyridin-3-yl)-5-fluorophenol;
4-(4-amino-7-(2,3-dihydro-1H-inden-2-yl)-7H-pyrrolo[2,3-d]pyrimidin-5-yl)phenol;
4-(4-amino-1-(2,3-dihydro-1H-inden-2-yl)-1H-pyrazolo[4,3-c]pyridin-3-yl)phenol;
6-bromo-5-(3,4-difluoro-5-methoxyphenyl)-7-(2,3-dihydro-1H-inden-2-yl)-7H-pyrrolo[2,3-d]pyrimidin-4-amine;
4-amino-5-(3,4-difluoro-5-methoxyphenyl)-7-(2,3-dihydro-1H-inden-2-yl)-7H-pyrrolo[2,3-d]pyrimidine-6-carbonitrile;
6-chloro-5-(3,4-difluoro-5-methoxyphenyl)-7-(2,3-dihydro-1H-inden-2-yl)-7H-pyrrolo[2,3-d]pyrimidin-4-amine;
5-(3,4-difluoro-5-methoxyphenyl)-7-(2,3-dihydro-1H-inden-2-yl)-5H-pyrrolo[3,2-d]pyrimidin-4-amine;
5-(4-amino-1-(2,3-dihydro-1H-inden-2-yl)-1H-pyrazolo[3,4-d]pyrimidin-3-yl)-3-fluoro-2-(trifluoromethyl)phenol;
1-(2,3-dihydro-1H-inden-2-yl)-3-(6-fluoro-5-phenoxy-1H-indol-2-yl)-1H-pyrazolo[3,4-d]pyrimidin-4-amine;
5-(4-amino-1-(2,3-dihydro-1H-inden-2-yl)-1H-pyrazolo[3,4-d]pyrimidin-3-yl)-3-hydroxy-2-(trifluoromethyl)benzonitrile;
5-(4-amino-1-(2,3-dihydro-1H-inden-2-yl)-1H-pyrazolo[3,4-d]pyrimidin-3-yl)-2-fluoro-3-methoxybenzonitrile;
5-(4-amino-1-(5-fluoro-2,3-dihydro-1H-inden-2-yl)-1H-pyrazolo[3,4-d]pyrimidin-3-yl)-2-fluoro-3-(trifluoromethyl)phenol;
5-(4-amino-1-(5-fluoro-2,3-dihydro-1H-inden-2-yl)-1H-pyrazolo[3,4-d]pyrimidin-3-yl)-2-chloro-3-(trifluoromethyl)phenol;
5-(4-amino-1-(2,3-dihydro-1H-inden-1-yl)-1H-pyrazolo[3,4-d]pyrimidin-3-yl)-2,3-dichlorophenol;
1-(2,3-dihydro-1H-inden-2-yl)-3-(1H-pyrrolo[2,3-b]pyridin-5-yl)-1H-pyrazolo[3,4-d]pyrimidin-4-amine;
1-(2,3-dihydro-1H-inden-2-yl)-3-(5-fluoro-6-methoxypyridin-3-yl)-1H-pyrazolo[3,4-d]pyrimidin-4-amine;
3-(4-amino-1-(2-(vinylsulfonyl)-1,2,3,4-tetrahydroisoquinolin-4-yl)-1H-pyrazolo[3,4-d]pyrimidin-3-yl)-5-fluorophenol;
3-(4-amino-6-(methylamino)-1-(1,2,3,4-tetrahydronaphthalen-1-yl)-1H-pyrazolo[3,4-d]pyrimidin-3-yl)-5-fluorophenol;
3-(4-amino-6-(methylamino)-1-(1,2,3,4-tetrahydronaphthalen-2-yl)-1H-pyrazolo[3,4-d]pyrimidin-3-yl)-5-fluorophenol;
N-(6-(4-amino-3-(3-fluoro-5-hydroxyphenyl)-1H-pyrazolo[3,4-d]pyrimidin-1-yl)-5,6,7,8-tetrahydronaphthalen-1-yl)acrylamide;
(E)-1-(3-(4-amino-3-(3-fluoro-5-hydroxyphenyl)-1H-pyrazolo[3,4-d]pyrimidin-1-yl)-3,4-dihydroquinolin-1(2H)-yl)-3-(dimethylamino)prop-2-en-1-one;
1-(3-(4-amino-3-(3-fluoro-5-hydroxyphenyl)-1H-pyrazolo[3,4-d]pyrimidin-1-yl)-3,4-dihydroquinolin-1(2H)-yl)prop-2-yn-1-one;
1-(3-(4-amino-3-(3-fluoro-5-hydroxyphenyl)-1H-pyrazolo[3,4-d]pyrimidin-1-yl)-3,4-dihydroquinolin-1(2H)-yl)but-2-yn-1-one;
1-(3-(4-amino-3-(3-fluoro-5-hydroxyphenyl)-1H-pyrazolo[3,4-d]pyrimidin-1-yl)-3,4-dihydroquinolin-1(2H)-yl)-3-(dimethylamino)prop-2-yn-1-one;
3-(4-amino-1-(8-methyl-1,2,3,4-tetrahydronaphthalen-1-yl)-1H-pyrazolo[3,4-d]pyrimidin-3-yl)-5-fluorophenol;
3-(4-amino-1-(7-methyl-1,2,3,4-tetrahydronaphthalen-1-yl)-1H-pyrazolo[3,4-d]pyrimidin-3-yl)-5-fluorophenol;
3-(4-amino-1-(6-methyl-1,2,3,4-tetrahydronaphthalen-1-yl)-1H-pyrazolo[3,4-d]pyrimidin-3-yl)-5-fluorophenol;
3-(4-amino-1-(5-methyl-1,2,3,4-tetrahydronaphthalen-1-yl)-1H-pyrazolo[3,4-d]pyrimidin-3-yl)-5-fluorophenol;
3-(4-amino-1-(8-fluoro-1,2,3,4-tetrahydronaphthalen-1-yl)-1H-pyrazolo[3,4-d]pyrimidin-3-yl)-5-fluorophenol;
5-(4-amino-1-(1,2,3,4-tetrahydronaphthalen-1-yl)-1H-pyrazolo[3,4-d]pyrimidin-3-yl)-3-chloro-2-fluorophenol;
3-(4-amino-1-(6-fluoro-1,2,3,4-tetrahydronaphthalen-1-yl)-1H-pyrazolo[3,4-d]pyrimidin-3-yl)-5-fluorophenol;
3-(4-amino-1-(5-fluoro-1,2,3,4-tetrahydronaphthalen-1-yl)-1H-pyrazolo[3,4-d]pyrimidin-3-yl)-5-fluorophenol;
8-(4-amino-3-(3-fluoro-5-hydroxyphenyl)-1H-pyrazolo[3,4-d]pyrimidin-1-yl)-5,6,7,8-tetrahydronaphthalen-1-ol;
8-(4-amino-3-(3-fluoro-5-hydroxyphenyl)-1H-pyrazolo[3,4-d]pyrimidin-1-yl)-5,6,7,8-tetrahydronaphthalen-2-ol;
5-(4-amino-3-(3-fluoro-5-hydroxyphenyl)-1H-pyrazolo[3,4-d]pyrimidin-1-yl)-5,6,7,8-tetrahydronaphthalen-2-ol;
5-(4-amino-3-(3-fluoro-5-hydroxyphenyl)-1H-pyrazolo[3,4-d]pyrimidin-1-yl)-5,6,7,8-tetrahydronaphthalen-1-ol;
3-(4-amino-1-(8-cyclopropyl-1,2,3,4-tetrahydronaphthalen-1-yl)-1H-pyrazolo[3,4-d]pyrimidin-3-yl)-5-fluorophenol;
3-(4-amino-1-(7-cyclopropyl-1,2,3,4-tetrahydronaphthalen-1-yl)-1H-pyrazolo[3,4-d]pyrimidin-3-yl)-5-fluorophenol;
3-(4-amino-1-(6-cyclopropyl-1,2,3,4-tetrahydronaphthalen-1-yl)-1H-pyrazolo[3,4-d]pyrimidin-3-yl)-5-fluorophenol;
3-(4-amino-1-(5-cyclopropyl-1,2,3,4-tetrahydronaphthalen-1-yl)-1H-pyrazolo[3,4-d]pyrimidin-3-yl)-5-fluorophenol;
3-(4-amino-1-(7-(tetrahydro-2H-pyran-4-yl)-1,2,3,4-tetrahydronaphthalen-1-yl)-1H-pyrazolo[3,4-d]pyrimidin-3-yl)-5-fluorophenol;
3-(4-amino-1-(7-(1-methylpiperidin-4-yl)-1,2,3,4-tetrahydronaphthalen-1-yl)-1H-pyrazolo[3,4-d]pyrimidin-3-yl)-5-fluorophenol;
3-(4-amino-1-(7-morpholino-1,2,3,4-tetrahydronaphthalen-1-yl)-1H-pyrazolo[3,4-d]pyrimidin-3-yl)-5-fluorophenol;
(E)-1-(3-(4-amino-3-(3-fluoro-5-hydroxyphenyl)-1H-pyrazolo[3,4-d]pyrimidin-1-yl)-3,4-dihydroquinolin-1(2H)-yl)-3-morpholinoprop-2-en-1-one;
(E)-1-(3-(4-amino-3-(3-fluoro-5-hydroxyphenyl)-1H-pyrazolo[3,4-d]pyrimidin-1-yl)-3,4-dihydroquinolin-1(2H)-yl)-3-(pyrrolidin-1-yl)prop-2-en-1-one;
5-(4-amino-1-(4-fluoro-2,3-dihydro-1H-inden-2-yl)-1H-pyrazolo[3,4-d]pyrimidin-3-yl)-2,3-difluorophenol;
(E)-3-(4-amino-1-(1-(prop-1-enylsulfonyl)-1,2,3,4-tetrahydroquinolin-3-yl)-1H-pyrazolo[3,4-d]pyrimidin-3-yl)-5-fluorophenol;
(E)-3-(4-amino-1-(1-(3-(dimethylamino)prop-1-enylsulfonyl)-1,2,3,4-tetrahydroquinolin-3-yl)-1H-pyrazolo[3,4-d]pyrimidin-3-yl)-5-fluorophenol;
1-(3-(4-amino-3-(3-fluoro-5-hydroxyphenyl)-1H-pyrazolo[3,4-d]pyrimidin-1-yl)-3,4-dihydroquinolin-1(2H)-yl)buta-2,3-dien-1-one;
3-(4-amino-1-(1-(propa-1,2-dienylsulfonyl)-1,2,3,4-tetrahydroquinolin-3-yl)-1H-pyrazolo[3,4-d]pyrimidin-3-yl)-5-fluorophenol;
N-(8-(4-amino-3-(3-fluoro-5-hydroxyphenyl)-1H-pyrazolo[3,4-d]pyrimidin-1-yl)-5,6,7,8-tetrahydronaphthalen-1-yl)acrylamide;
N-(8-(4-amino-3-(3-fluoro-5-hydroxyphenyl)-1H-pyrazolo[3,4-d]pyrimidin-1-yl)-5,6,7,8-tetrahydronaphthalen-2-yl)acrylamide;
N-(5-(4-amino-3-(3-fluoro-5-hydroxyphenyl)-1H-pyrazolo[3,4-d]pyrimidin-1-yl)-5,6,7,8-tetrahydronaphthalen-2-yl)acrylamide;
N-(5-(4-amino-3-(3-fluoro-5-hydroxyphenyl)-1H-pyrazolo[3,4-d]pyrimidin-1-yl)-5,6,7,8-tetrahydronaphthalen-1-yl)acrylamide;
3-(4-amino-1-(4-fluoro-2,3-dihydro-1H-inden-2-yl)-1H-pyrazolo[3,4-d]pyrimidin-3-yl)-5-fluorophenol;
2-(4-amino-3-(3-fluoro-5-hydroxyphenyl)-1H-pyrazolo[3,4-d]pyrimidin-1-yl)-2,3-dihydro-1H-inden-4-ol;
3-(4-amino-1-(4-cyclopropyl-2,3-dihydro-1H-inden-2-yl)-1H-pyrazolo[3,4-d]pyrimidin-3-yl)-5-fluorophenol;
3-(3-(allyloxy)-5-fluorophenyl)-1-(1,2,3,4-tetrahydronaphthalen-2-yl)-1H-pyrazolo[3,4-d]pyrimidin-4-amine;
1-(3-(3-(3-(allyloxy)-5-fluorophenyl)-4-amino-1H-pyrazolo[3,4-d]pyrimidin-1-yl)-3,4-dihydroquinolin-1(2H)-yl)prop-2-en-1-one;
1-(4-(4-amino-1-(1,2,3,4-tetrahydronaphthalen-2-yl)-1H-pyrazolo[3,4-d]pyrimidin-3-yl)piperazin-1-yl)prop-2-en-1-one;
1-(4-(4-amino-1-(1,2,3,4-tetrahydronaphthalen-2-yl)-1H-pyrazolo[3,4-d]pyrimidin-3-ylamino)piperidin-1-yl)prop-2-en-1-one;
1-(4-(4-amino-1-(1,2,3,4-tetrahydronaphthalen-2-yl)-1H-pyrazolo[3,4-d]pyrimidin-3-ylthio)piperidin-1-yl)prop-2-en-1-one;
1-(4-(4-amino-1-(1,2,3,4-tetrahydronaphthalen-2-yl)-1H-pyrazolo[3,4-d]pyrimidin-3-yloxy)piperidin-1-yl)prop-2-en-1-one;
N-(3-(4-amino-1-(1,2,3,4-tetrahydronaphthalen-2-yl)-1H-pyrazolo[3,4-d]pyrimidin-3-yl)-5-hydroxyphenyl)acrylamide;
N-(4-(4-amino-1-(1,2,3,4-tetrahydronaphthalen-2-yl)-1H-pyrazolo[3,4-d]pyrimidin-3-yl)-2-hydroxyphenyl)acrylamide;
N-(5-(1-(1-acryloyl-1,2,3,4-tetrahydroquinolin-3-yl)-4-amino-1H-pyrazolo[3,4-d]pyrimidin-3-yl)-2-hydroxyphenyl)acrylamide;
N-(3-(1-(1-acryloyl-1,2,3,4-tetrahydroquinolin-3-yl)-4-amino-1H-pyrazolo[3,4-d]pyrimidin-3-yl)-5-hydroxyphenyl)acrylamide;
N-(4-(1-(1-acryloyl-1,2,3,4-tetrahydroquinolin-3-yl)-4-amino-1H-pyrazolo[3,4-d]pyrimidin-3-yl)-2-hydroxyphenyl)acrylamide;
1-(3-(4-amino-3-(3,4-difluoro-5-hydroxyphenyl)-1H-pyrazolo[3,4-d]pyrimidin-1-yl)-3,4-dihydroquinolin-1(2H)-yl)prop-2-en-1-one;
1-(3-(4-amino-3-(1H-indol-1-yl)-1H-pyrazolo[3,4-d]pyrimidin-1-yl)-3,4-dihydroquinolin-1(2H)-yl)prop-2-en-1-one;
1-(3-(4-amino-3-(5-fluoro-7-hydroxy-1H-indol-1-yl)-1H-pyrazolo[3,4-d]pyrimidin-1-yl)-3,4-dihydroquinolin-1(2H)-yl)prop-2-en-1-one;
1-(3-(4-amino-3-(5-fluoro-6-hydroxy-1H-indol-1-yl)-1H-pyrazolo[3,4-d]pyrimidin-1-yl)-3,4-dihydroquinolin-1(2H)-yl)prop-2-en-1-one;
1-(3-(4-amino-3-(6-fluoro-5-hydroxy-1H-indol-1-yl)-1H-pyrazolo[3,4-d]pyrimidin-1-yl)-3,4-dihydroquinolin-1(2H)-yl)prop-2-en-1-one;
3-(1H-indol-1-yl)-1-(1,2,3,4-tetrahydronaphthalen-2-yl)-1H-pyrazolo[3,4-d]pyrimidin-4-amine;
1-(4-amino-1-(1,2,3,4-tetrahydronaphthalen-2-yl)-1H-pyrazolo[3,4-d]pyrimidin-3-yl)-5-fluoro-1H-indol-7-ol;
1-(4-amino-1-(1,2,3,4-tetrahydronaphthalen-2-yl)-1H-pyrazolo[3,4-d]pyrimidin-3-yl)-5-fluoro-1H-indol-6-ol;
1-(4-amino-1-(1,2,3,4-tetrahydronaphthalen-2-yl)-1H-pyrazolo[3,4-d]pyrimidin-3-yl)-6-fluoro-1H-indol-5-ol;
1-(2,3-dihydro-1H-inden-2-yl)-3-(1H-indol-1-yl)-1H-pyrazolo[3,4-d]pyrimidin-4-amine;
1-(4-amino-1-(2,3-dihydro-1H-inden-2-yl)-1H-pyrazolo[3,4-d]pyrimidin-3-yl)-5-fluoro-1H-indol-7-ol;
1-(4-amino-1-(2,3-dihydro-1H-inden-2-yl)-1H-pyrazolo[3,4-d]pyrimidin-3-yl)-5-fluoro-1H-indol-6-ol;
1-(4-amino-1-(2,3-dihydro-1H-inden-2-yl)-1H-pyrazolo[3,4-d]pyrimidin-3-yl)-6-fluoro-1H-indol-5-ol;
3-(1H-indol-2-yl)-1-(1,2,3,4-tetrahydronaphthalen-2-yl)-1H-pyrazolo[3,4-d]pyrimidin-4-amine;
2-(4-amino-1-(1,2,3,4-tetrahydronaphthalen-2-yl)-1H-pyrazolo[3,4-d]pyrimidin-3-yl)-5-fluoro-1H-indol-7-ol;
2-(4-amino-1-(1,2,3,4-tetrahydronaphthalen-2-yl)-1H-pyrazolo[3,4-d]pyrimidin-3-yl)-5-fluoro-1H-indol-6-ol;
2-(4-amino-1-(1,2,3,4-tetrahydronaphthalen-2-yl)-1H-pyrazolo[3,4-d]pyrimidin-3-yl)-6-fluoro-1H-indol-5-ol;
1-(3-(4-amino-3-(1H-indol-2-yl)-1H-pyrazolo[3,4-d]pyrimidin-1-yl)-3,4-dihydroquinolin-1(2H)-yl)prop-2-en-1-one;
1-(3-(4-amino-3-(5-fluoro-7-hydroxy-1H-indol-2-yl)-1H-pyrazolo[3,4-d]pyrimidin-1-yl)-3,4-dihydroquinolin-1(2H)-yl)prop-2-en-1-one;
1-(3-(4-amino-3-(5-fluoro-6-hydroxy-1H-indol-2-yl)-1H-pyrazolo[3,4-d]pyrimidin-1-yl)-3,4-dihydroquinolin-1(2H)-yl)prop-2-en-1-one;
1-(3-(4-amino-3-(6-fluoro-5-hydroxy-1H-indol-2-yl)-1H-pyrazolo[3,4-d]pyrimidin-1-yl)-3,4-dihydroquinolin-1(2H)-yl)prop-2-en-1-one;
1-(2,3-dihydro-1H-inden-2-yl)-3-(1H-indol-2-yl)-1H-pyrazolo[3,4-d]pyrimidin-4-amine;
2-(4-amino-1-(2,3-dihydro-1H-inden-2-yl)-1H-pyrazolo[3,4-d]pyrimidin-3-yl)-5-fluoro-1H-indol-7-ol;
2-(4-amino-1-(2,3-dihydro-1H-inden-2-yl)-1H-pyrazolo[3,4-d]pyrimidin-3-yl)-5-fluoro-1H-indol-6-ol;
2-(4-amino-1-(2,3-dihydro-1H-inden-2-yl)-1H-pyrazolo[3,4-d]pyrimidin-3-yl)-6-fluoro-1H-indol-5-ol;
1-(3-(3-(4-(allyloxy)-3-hydroxyphenyl)-4-amino-1H-pyrazolo[3,4-d]pyrimidin-1-yl)-3,4-dihydroquinolin-1(2H)-yl)prop-2-en-1-one;
1-(3-(3-(3-(allyloxy)-4-hydroxyphenyl)-4-amino-1H-pyrazolo[3,4-d]pyrimidin-1-yl)-3,4-dihydroquinolin-1(2H)-yl)prop-2-en-1-one;
1-(3-(3-(3-(allyloxy)-5-hydroxyphenyl)-4-amino-1H-pyrazolo[3,4-d]pyrimidin-1-yl)-3,4-dihydroquinolin-1(2H)-yl)prop-2-en-1-one;
1-(3-(3-(4-(allyloxy)phenyl)-4-amino-1H-pyrazolo[3,4-d]pyrimidin-1-yl)-3,4-dihydroquinolin-1(2H)-yl)prop-2-en-1-one;
2-(allyloxy)-5-(4-amino-1-(1,2,3,4-tetrahydronaphthalen-2-yl)-1H-pyrazolo[3,4-d]pyrimidin-3-yl)phenol;
2-(allyloxy)-4-(4-amino-1-(1,2,3,4-tetrahydronaphthalen-2-yl)-1H-pyrazolo[3,4-d]pyrimidin-3-yl)phenol;
3-(allyloxy)-5-(4-amino-1-(1,2,3,4-tetrahydronaphthalen-2-yl)-1H-pyrazolo[3,4-d]pyrimidin-3-yl)phenol;
3-(4-(allyloxy)phenyl)-1-(1,2,3,4-tetrahydronaphthalen-2-yl)-1H-pyrazolo[3,4-d]pyrimidin-4-amine;
2-(allyloxy)-5-(4-amino-1-(2,3-dihydro-1H-inden-2-yl)-1H-pyrazolo[3,4-d]pyrimidin-3-yl)phenol;
2-(allyloxy)-4-(4-amino-1-(2,3-dihydro-1H-inden-2-yl)-1H-pyrazolo[3,4-d]pyrimidin-3-yl)phenol;
3-(allyloxy)-5-(4-amino-1-(2,3-dihydro-1H-inden-2-yl)-1H-pyrazolo[3,4-d]pyrimidin-3-yl)phenol;
3-(4-(allyloxy)phenyl)-1-(2,3-dihydro-1H-inden-2-yl)-1H-pyrazolo[3,4-d]pyrimidin-4-amine;
5-(1H-indol-1-yl)-7-(1,2,3,4-tetrahydronaphthalen-2-yl)-7H-pyrrolo[2,3-d]pyrimidin-4-amine;
1-(4-amino-7-(1,2,3,4-tetrahydronaphthalen-2-yl)-7H-pyrrolo[2,3-d]pyrimidin-5-yl)-5-fluoro-1H-indol-7-ol;
1-(4-amino-7-(1,2,3,4-tetrahydronaphthalen-2-yl)-7H-pyrrolo[2,3-d]pyrimidin-5-yl)-5-fluoro-1H-indol-6-ol;
1-(4-amino-7-(1,2,3,4-tetrahydronaphthalen-2-yl)-7H-pyrrolo[2,3-d]pyrimidin-5-yl)-6-fluoro-1H-indol-5-ol;
7-(2,3-dihydro-1H-inden-2-yl)-5-(1H-indol-1-yl)-7H-pyrrolo[2,3-d]pyrimidin-4-amine;
1-(4-amino-7-(2,3-dihydro-1H-inden-2-yl)-7H-pyrrolo[2,3-d]pyrimidin-5-yl)-5-fluoro-1H-indol-7-ol;
1-(4-amino-7-(2,3-dihydro-1H-inden-2-yl)-7H-pyrrolo[2,3-d]pyrimidin-5-yl)-5-fluoro-1H-indol-6-ol;
1-(4-amino-7-(2,3-dihydro-1H-inden-2-yl)-7H-pyrrolo[2,3-d]pyrimidin-5-yl)-6-fluoro-1H-indol-5-ol;
5-(1H-indol-2-yl)-7-(1,2,3,4-tetrahydronaphthalen-2-yl)-7H-pyrrolo[2,3-d]pyrimidin-4-amine;
2-(4-amino-7-(1,2,3,4-tetrahydronaphthalen-2-yl)-7H-pyrrolo[2,3-d]pyrimidin-5-yl)-5-fluoro-1H-indol-7-ol;
2-(4-amino-7-(1,2,3,4-tetrahydronaphthalen-2-yl)-7H-pyrrolo[2,3-d]pyrimidin-5-yl)-5-fluoro-1H-indol-6-ol;
2-(4-amino-7-(1,2,3,4-tetrahydronaphthalen-2-yl)-7H-pyrrolo[2,3-d]pyrimidin-5-yl)-6-fluoro-1H-indol-5-ol;
7-(2,3-dihydro-1H-inden-2-yl)-5-(1H-indol-2-yl)-7H-pyrrolo[2,3-d]pyrimidin-4-amine;
2-(4-amino-7-(2,3-dihydro-1H-inden-2-yl)-7H-pyrrolo[2,3-d]pyrimidin-5-yl)-5-fluoro-1H-indol-7-ol;
2-(4-amino-7-(2,3-dihydro-1H-inden-2-yl)-7H-pyrrolo[2,3-d]pyrimidin-5-yl)-5-fluoro-1H-indol-6-ol;
2-(4-amino-7-(2,3-dihydro-1H-inden-2-yl)-7H-pyrrolo[2,3-d]pyrimidin-5-yl)-6-fluoro-1H-indol-5-ol;
1-(3-(3-(6-(allyloxy)-5-fluoro-1H-indol-1-yl)-4-amino-1H-pyrazolo[3,4-d]pyrimidin-1-yl)-3,4-dihydroquinolin-1(2H)-yl)prop-2-en-1-one;
1-(3-(3-(5-(allyloxy)-6-fluoro-1H-indol-1-yl)-4-amino-1H-pyrazolo[3,4-d]pyrimidin-1-yl)-3,4-dihydroquinolin-1(2H)-yl)prop-2-en-1-one;
3-(6-(allyloxy)-5-fluoro-1H-indol-1-yl)-1-(1,2,3,4-tetrahydronaphthalen-2-yl)-1H-pyrazolo[3,4-d]pyrimidin-4-amine;
3-(5-(allyloxy)-6-fluoro-1H-indol-1-yl)-1-(1,2,3,4-tetrahydronaphthalen-2-yl)-1H-pyrazolo[3,4-d]pyrimidin-4-amine;
3-(6-(allyloxy)-5-fluoro-1H-indol-1-yl)-1-(2,3-dihydro-1H-inden-2-yl)-1H-pyrazolo[3,4-d]pyrimidin-4-amine;
3-(5-(allyloxy)-6-fluoro-1H-indol-1-yl)-1-(2,3-dihydro-1H-inden-2-yl)-1H-pyrazolo[3,4-d]pyrimidin-4-amine;
1-(4-amino-3-(2-hydroxyphenylamino)-1H-pyrazolo[3,4-d]pyrimidin-1-yl)-2,3-dihydro-1H-inden-4-ol;
1-(4-amino-3-(3-fluoro-5-hydroxyphenyl)-1H-pyrazolo[3,4-d]pyrimidin-1-yl)-2,3-dihydro-1H-inden-4-ol;
3-(4-amino-1-(1,2,3,4-tetrahydroquinolin-3-yl)-1H-pyrazolo[3,4-d]pyrimidin-3-yl)phenol;
5-(4-amino-1-(1,2,3,4-tetrahydroquinolin-3-yl)-1H-pyrazolo[3,4-d]pyrimidin-3-yl)-2,3-difluorophenol;
(4-(4-amino-1-(1,2,3,4-tetrahydronaphthalen-1-yl)-1H-pyrazolo[3,4-d]pyrimidin-3-yl)phenyl)methanol;
3-(4-amino-1-(1,2,3,4-tetrahydronaphthalen-2-yl)-1H-pyrazolo[3,4-d]pyrimidin-3-yl)-5-fluorophenyl acrylate;
2-amino-5-(4-amino-1-(1,2,3,4-tetrahydronaphthalen-2-yl)-1H-pyrazolo[3,4-d]pyrimidin-3-yl)phenol;
5-(4-amino-7-(1,2,3,4-tetrahydronaphthalen-2-yl)imidazo[1,5-f][1,2,4]triazin-5-yl)-2-(trifluoromethyl)phenol;
3-(4-amino-7-(1,2,3,4-tetrahydronaphthalen-2-yl)imidazo[1,5-f][1,2,4]triazin-5-yl)-5-fluorophenol;
3-(4-amino-7-(1,2,3,4-tetrahydronaphthalen-1-yl)imidazo[1,5-f][1,2,4]triazin-5-yl)-5-fluorophenol;
3-(4-amino-7-(2,3-dihydro-1H-inden-2-yl)imidazo[1,5-f][1,2,4]triazin-5-yl)-5-fluorophenol;
3-(4-amino-7-(2,3-dihydro-1H-inden-1-yl)imidazo[1,5-f][1,2,4]triazin-5-yl)-5-fluorophenol;
3-amino-5-(4-amino-1-(1,2,3,4-tetrahydronaphthalen-2-yl)-1H-pyrazolo[3,4-d]pyrimidin-3-yl)phenol;
1-(4-(4-amino-1-(1,2,3,4-tetrahydronaphthalen-1-yl)-1H-pyrazolo[3,4-d]pyrimidin-3-yl)-2-fluorophenyl)prop-2-en-1-one;
1-(3-(4-amino-1-(1,2,3,4-tetrahydronaphthalen-1-yl)-1H-pyrazolo[3,4-d]pyrimidin-3-yl)-5-fluorophenyl)prop-2-en-1-one;
1-(5-(4-amino-3-(3-fluoro-5-hydroxyphenyl)-1H-pyrazolo[3,4-d]pyrimidin-1-yl)-5,6,7,8-tetrahydronaphthalen-1-yl)prop-2-en-1-one;
1-(2-(4-amino-3-(3-fluoro-5-hydroxyphenyl)-1H-pyrazolo[3,4-d]pyrimidin-1-yl)-2,3-dihydro-1H-inden-4-yl)prop-2-en-1-one;
3-(4-amino-1-(4-(vinylsulfonyl)-2,3-dihydro-1H-inden-2-yl)-1H-pyrazolo[3,4-d]pyrimidin-3-yl)-5-fluorophenol;
2-(3-(4-amino-3-(3-fluoro-5-hydroxyphenyl)-1H-pyrazolo[3,4-d]pyrimidin-1-yl)-1,2,3,4-tetrahydroquinoline-1-carbonyl)acrylonitrile;
1-(3-(4-amino-3-(3-fluoro-5-hydroxyphenyl)-1H-pyrazolo[3,4-d]pyrimidin-1-yl)-3,4-dihydroquinolin-1(2H)-yl)-2-(methylsulfonyl)prop-2-en-1-one;
2-(3-(4-amino-3-(3-fluoro-5-hydroxyphenyl)-1H-pyrazolo[3,4-d]pyrimidin-1-yl)-1,2,3,4-tetrahydroquinoline-1-carbonyl)acrylamide;
1-(3-(4-amino-3-(3-fluoro-5-hydroxyphenyl)-1H-pyrazolo[3,4-d]pyrimidin-1-yl)-3,4-dihydro-1,6-naphthyridin-1(2H)-yl)prop-2-en-1-one;
1-(3-(4-amino-3-(3-fluoro-5-hydroxyphenyl)-1H-pyrazolo[3,4-d]pyrimidin-1-yl)-3,4-dihydro-1,5-naphthyridin-1(2H)-yl)prop-2-en-1-one; and
2-(3-(4-amino-3-(3-fluoro-5-hydroxyphenyl)-1H-pyrazolo[3,4-d]pyrimidin-1-yl)-1,2,3,4-tetrahydro-1,5-naphthyridine-1-carbonyl)acrylonitrile, and
pharmaceutically acceptable salts thereof.

28. A pharmaceutical composition comprising a compound of claim 1, and a pharmaceutically acceptable carrier.

29. A method for the treatment of cancer or for the treatment of an autoimmune disease to a patient comprising the administration of a therapeutically effective amount of a Btk inhibitor to a patient in need thereof, wherein the Btk inhibitor is a compound of the formulae (I), (II) or (III), or any one of a compound of the formula A-1 to A-10, A-1a to A-10e, A-1a-1 to A-10e-8, (B-1), (B-2), (B-3), or (B-4); or a compound of Table 1 or 2, or a pharmaceutically acceptable salt thereof.

30. The method of claim 29, wherein the cancer is selected from the group consisting of chronic lymphocytic leukemia, small lymphocytic leukemia, mantle cell lymphoma, diffuse large B cell lymphoma, multiple myeloma, B cell non Hodgkin lymphoma and acute myeloid lymphoma.

31. The method of claim 29, wherein the autoimmune disease is selected from the group consisting of rheumatoid arthritis and systemic lupus erythematosis.

32. A method for the treatment of cancer or for the treatment of an autoimmune disease to a patient comprising the administration of a therapeutically effective amount of a PI3Kδ inhibitor to a patient in need thereof, wherein the PI3Kδ inhibitor is a compound of the formulae (I), (II) or (III), or any one of a compound of the formula A-1 to A-10, A-1a to A-10e, A-1a-1 to A-10e-8, (B-1), (B-2), (B-3), or (B-4); or a compound of Table 1 or 2, or a pharmaceutically acceptable salt thereof.

33. The method of claim 32, wherein the cancer is selected from the group consisting of chronic lymphocytic leukemia, mantle cell lymphoma, B cell non Hodgkin lymphoma, multiple myeloma and acute myeloid lymphoma.

34. The method of claim 32, wherein the autoimmune disease is selected from the group consisting of rheumatoid arthritis, allergic asthma and myocardial infarction.

35. A method for the treatment of cancer or for the treatment of an autoimmune disease to a patient comprising the administration of a therapeutically effective amount of a dual Btk and PI3Kδ inhibitor to a patient in need thereof, wherein the dual Btk and PI3Kδ inhibitor is a compound of the formulae (I), (II) or (III), or any one of a compound of the formula A-1 to A-10, A-1a to A-10e, A-1a-1 to A-10e-8, (B-1), (B-2), (B-3), or (B-4); or a compound of Table 1 or 2, or a pharmaceutically acceptable salt thereof.

36. The method of claim 35, wherein the cancer is selected from the group consisting of chronic lymphocytic leukemia, mantle cell lymphoma, multiple myeloma and B cell non Hodgkin lymphoma.

37. The method of claim 35, wherein the autoimmune disease is rheumatoid arthritis.

38. A kit comprising (i) a compound or composition of claim 1, or a pharmaceutically acceptable salt thereof, and (ii) instructions for use in a disease or condition.
